(19) United States
(12) Patent Application Publication
(10) Pub. No.: US 2026/0234144 A1
(43) Pub. Date: Aug. 13, 2026

(54) COMPOUNDS COMPRISING A NAPHTHYRIDINE OR PYRIDOPYRIMIDINE CORE AS PTC READ-THROUGH AGENTS

(71) Applicant: TAY THERAPEUTICS LIMITED, Dundee, DD1 5jj (GB)

(72) Inventors: Christopher Andrew WOODLAND, Dundee, Tayside (GB); Mark BELL, Dundee, Tayside (GB); David FOLEY, Dundee, Tayside (GB)

(73) Assignee: TAY THERAPEUTICS LIMITED, Dundee, Tayside (GB)

(21) Appl. No.: 19/501,061

(22) PCT Filed: Jul. 10, 2024
(86) PCT No.: PCT/GB2024/051804

§ 371 Date: Jan. 9, 2026

(30) Foreign Application Priority Data
Jul. 11, 2023 (GB) 2310623.0
Dec. 21, 2023 (GB) 2319859.1

(51) Int. Cl. C07D 471/04; A61K 31/519; A61K 31/5375; A61K 31/55; C07B 59/00; C07D 519/00

(52) U.S. Cl. CPC C07D 471/04; A61K 31/519; A61K 31/5375; A61K 31/55; C07B 59/002; C07D 519/00; C07B 2200/05

(57) ABSTRACT

Disclosed are compounds of the formula (I), or a tautomeric form thereof, or a pharmaceutically acceptable salt or N-oxide thereof: (I) wherein R¹, R^{2a}, R^{2b}, R³, R⁴, R⁵, and X are as defined herein. Compounds of the invention may be suitable for use in treating diseases/conditions which are associated with PTC mutations. Also disclosed are pharmaceutical compositions comprising the compounds; and the compounds for use in treating conditions or disorders which are associated with PTC mutations in a subject.

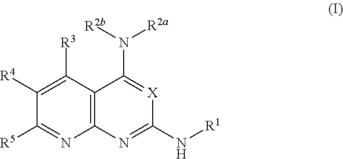

This invention relates to compounds comprising a pyridine-containing polycyclic core having pharmacological activity, processes for their preparation, pharmaceutical compositions containing them and their use in the treatment of various disorders.

### BACKGROUND

When a ribosome encounters a termination sequence during the process of messenger RNA (mRNA) translation no cognate transfer RNA (tRNA) can bind. This leads to a halt in protein production, release factor (RF) proteins binding to the empty site on the ribosome and conformational changes in the ribosome that lead to the break-up of the ribosomal complex. Although accurate, translational termination is not 100% effective and its efficiency depends on competition between the recognition of the stop codon by eRF1, and the decoding of the stop codon by a near-cognate tRNA (i.e. a natural suppressor tRNA). The latter case leads to the suppression of translation termination, also called “read-through”, where an amino acid is incorporated in place of the stop (Dabrowski, M. et al., RNA Biology 2015, 12, 950-958).

If the terminating codon occurs before the full length protein is made then the termination codon is a “premature” termination codon (PTC) and the truncated protein may not be functional. Normal terminating stop codons are usually in a genetic and structural context that reinforces their effectiveness and are usually more resistant to read-through than PTCs arising through truncating mutations (Wangen and Green, 2020 Elife 9:e52611).

Over 1800 separate inherited genetic disorders have been identified where the causative mutation in a proportion of individuals is a nonsense mutation where an in-frame, premature termination codon (PTC) leads to a loss of gene function (Kellermayer, R. European Journal of Medical Genetics 2006, 49, 445-450). Over 9,600 PTC mutations have been published, representing around 10% of all reported gene mutations (Krawczak, M. et al., Human Mutation 2000, 15, 45-51, Mort, M. et al., Human Mutation 2008, 29, 1037-1047).

Nonsense mutations are involved in the pathology of a range of disorders; from polygenic pathologies such as cancer to monogenic diseases such as cystic fibrosis and Duchenne muscular dystrophy for which a PTC accounts for around 10% or 5-10% of all cases respectively (Keeling, K. M. & Bedwell, D. M., Journal of Molecular Medicine 2002, 80, 367-376, Kellermayer, R. European Journal of Medical Genetics 2006, 49, 445-450).

Whereas monogenetic diseases arise from either an inherited or de novo germline mutation resulting in a PTC in, for example cancer, somatic mutations can give rise to PTC mutations in tumour suppressor genes, for example.

One of the surveillance systems used by the cell to remove abnormal mRNA transcripts that prematurely terminate translation is the nonsense mediated decay (NMD) pathway. It targets mRNA containing a PTC sequence more than ~50 base pairs upstream from the penultimate exon-exon junction (Maquat, L. E., Nature Reviews, Molecular Cell biology 2004, 5, 89-99).

The combination of premature termination of protein production and NMD of mRNA containing PTCs, can lead to almost total loss of protein production for mutant genes encoding a PTC.

Mechanisms by which translational read-though of PTC mutations may be achieved include but are not limited to: reducing the translational fidelity of the ribosome; reducing the efficiency of the translational termination machinery; inhibiting NMD; interfering with the regulation of gene transcription or translation or stabilising mRNA against NMD (Kellermayer, R European Journal of Medical Genetics 2006, 49, 445-450, Belgrader, P. J. C., and Maquat, L. E., Proc. Natl. Acad. Sci. USA 1993, 90, 482-486).

Agents which allow translational read-through of PTC mutations have the potential to treat any disease where the presence of a PTC in a gene is directly causative, a risk factor or an aggravating factor in disease.

Aminoglycosides (such as paromomycin, gentamicin, G418/geneticin) are a class of antibiotics able to reduce translational fidelity. They exert their antibacterial action through inhibition of prokaryotic protein synthesis, however, at sub-lethal doses they lower translational fidelity of mammalian ribosomes leading to an increased rate of mis-incorporation of amino acids and also to an increased rate of translational read-through of stop codons (Davies, J. & Gorini, L., Proc. Natl. Acad. Sci. USA 1964, 51, 883-8, Weinstein, Proc. Natl. Acad. Sci. USA 1964, 52, 988-996).

Aminoglycosides have been shown to allow read-through of PTC mutations in the CFTR gene responsible for cystic fibrosis in vitro (Bedwell, D. M., et al., Nature Medicine 1997, 3, 1280-1284) and in vivo, including patients (Ming Du, et al., Journal of Molecular Medicine 2002, 80, 595-604, Clancy, J. P. et al., Am J Respir Crit Care Med 2001, 163, 1683-1692, Wilschanski, M., et al., The New England Journal of Medicine 2003, 139, 1433-1441).

In an animal model of Duchenne muscular dystrophy (DMD), 7 of 8 male mdx mice treated with 34 mg/kg gentamicin showed increased protection from contractile injury and 10-20% restoration of wild-type dystrophin levels (Barton-Davis, E. R., et al., Journal of Clinical Investigation 1999, 104, 375-381).

Gentamicin treatment induced functional type VII collagen in recessive dystrophic epidermolysis bullosa patients (Woodley, D. T. et al. Journal of Clinical Investigation 2017, 127, 3028-3038) with in vitro evidence supporting a read-through mechanism (Has, C. et al. Journal of Investigative Dermatology 2022, 142, 1227-1230).

The read-through of genes containing PTC mutations relevant to cystinosis, Hurler syndrome, inherited blindness and cancer genes have all been demonstrated following aminoglycoside treatment (Helip-Wooley, A., et al., Molecular Genetics and Metabolism 2002, 75, 128-133, Keeling, K. M., et al., Human Molecular Genetics 2001, 10, 291-299, Moosajee, M. K., et al., Human Molecular Genetics 2008, 17, 3987-4000). However, aminoglycosides are not suitable as PTC read-through agents due to the severe toxicity observed with high doses or repeated treatments

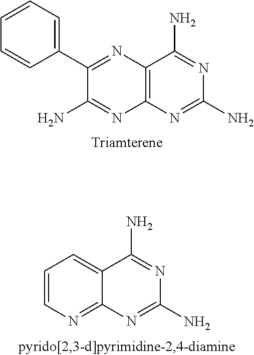 [SMILES: Nc1nc(N)c2nc(-c3ccccc3)c(N)nc2n1 ; Nc1nc(N)c2cccnc2n1]

WO2019241633 describes compositions and methods for suppressing nonsense mutations, including the compound triamterene.

Hurlbert, B. S. et al. Journal of Medicinal Chemistry (1968), 11(4), 711-17 describes 2,4-diaminopyrido[2,3-d]-pyrimidines, including pyrido[2,3-d]pyrimidine-2,4-diamine, with antibacterial and antiprotozoal effects.

Certain compounds of the present invention are suitable for use in treating diseases/conditions which are associated with PTC mutations.

A structurally novel class of compounds has now been found which provides PTC read-through agents.

### BRIEF SUMMARY OF THE DISCLOSURE

In accordance with a first aspect, the present invention provides a compound of formula (I), or a tautomeric form thereof, or a pharmaceutically acceptable salt or N-oxide thereof:

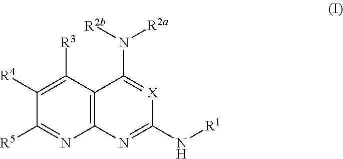

wherein
X is N or CR⁶;
R¹ is independently selected from C₀-C₆alkylene-Ria, and C₂-C₅-alkylene-R^{1b};
R^{1a} is independently selected from C₃-C₈ cycloalkyl, C₅-C₈ cycloalkenyl, 3- to 10-membered heterocycloalkyl, and 5- to 10-membered heterocycloalkenyl; wherein R^{1a} is optionally substituted with C₀-C₆-alkylene-R^{9a} and/or from 1 to 6 R⁹ groups; wherein where R^{1a} is heterocycloalkyl or heterocycloalkenyl, the heterocycloalkyl or heterocycloalkenyl is optionally fused to a phenyl ring, wherein the phenyl ring is optionally substituted with from 1 to 4 R¹⁰ groups;
R^{1b} is independently selected from NR^{1a}R^{8a} and OR⁷;
   wherein R¹ comprises at least one nitrogen atom;
R^{2a} is independently at each occurrence selected from H and C₁-C₄ alkyl;
R^{2b} is independently at each occurrence selected from H, C₁-C₄ alkyl, C₁-C₄-haloalkyl, C₀-C₄ alkyl-R^{2c}, C₂-C₄-alkylene-R^{2d}, C(O)—C₁-C₄-alkyl, S(O)—C₁-C₄-alkyl, and S(O)₂—C₁-C₄-alkyl;
R^{2c} is independently selected from C₃-C₈ cycloalkyl, C₅-C₈ cycloalkenyl, phenyl, 3- to 8-membered heterocycloalkyl, 5- to 8-membered heterocycloalkenyl and 5-, or 6-membered heteroaryl; wherein where R^{2c} is cycloalkyl, cycloalkenyl, heterocycloalkyl or heterocycloalkenyl, R^{2c} is optionally substituted with from 1 to 4 R⁹ groups; and where R^{2c} is phenyl, or heteroaryl, R^{5c} is optionally substituted with from 1 to 5 R¹⁰ groups;
R^{2d} is independently selected from NR⁷R⁸ and OR⁷;
   or R^{2a} and R^{2b}, together with the nitrogen atom to which they are attached, form a 5- to 8-membered heterocycloalkyl ring; optionally substituted with 1 to 4 R⁹ groups;
R³ is independently selected from H, cyano, C₁-C₄-alkylene-NR⁷R⁸, NR⁷R⁸, C₁-C₄-alkylene-OR⁷, OR⁷, SR⁷, SOR⁷, S(O)₂R⁷, S(O)₂NR⁷R⁷, CO₂R⁷, C(O)R⁷, C(O)NR⁷R⁷, C₁-C₄-alkyl, C₂-C₄-alkenyl, C₂-C₄-alkynyl, C₁-C₄-haloalkyl, NR⁷—C₀-C₄-alkylene-R^{3c}, O—C₀-C₄-alkylene-R^{3c}, and C₀-C₄-alkylene-R^{3c};
R^{3c} is independently selected from C₃-C₈ cycloalkyl, C₅-C₈ cycloalkenyl, phenyl, 3- to 10-membered heterocycloalkyl, 5- to 10-membered heterocycloalkenyl and 5-, or 6-membered heteroaryl; wherein where R^{3c} is cycloalkyl, cycloalkenyl, heterocycloalkyl or heterocycloalkenyl, R^{3c} is optionally substituted with from 1 to 4 R⁹ groups; and where R^{3C} is phenyl or heteroaryl, R^{3c} is optionally substituted with from 1 to 5 R¹⁰ groups;
R⁴ is independently selected from H, halo, cyano, NR^{1b}R⁸, OR⁷, SR⁷, SOR⁷, S(O)₂R⁷, S(O)₂NR⁷R⁷, CO₂R⁷, C(O)R⁷, C(O)NR⁷R⁷, C₁-C₄-alkyl, C₂-C₄-alkenyl, C₂-C₄-alkynyl, C₁-C₄-haloalkyl, O—C₀-C₄-alkylene-R^{4c}, and C₀-C₄-alkylene-R^{4c};
R^{4c} is independently selected from C₃-C₈ cycloalkyl, C₅-C₈ cycloalkenyl, 3- to 10-membered heterocycloalkyl, 5- to 10-membered heterocycloalkenyl and 5-membered heteroaryl; wherein where R^{4c} is cycloalkyl, cycloalkenyl, heterocycloalkyl or heterocycloalkenyl, R^{4c} is optionally substituted with from 1 to 4 R⁹ groups; and where R^{4c} is heteroaryl, R^{4c} is optionally substituted with from 1 to 5 R¹⁰ groups;
R⁵ is independently selected from H, halo, cyano, C₁-C₄-alkylene-NR⁷R⁸, NR^{7b}R⁸, C₁-C₄-alkylene-OR⁷, OR^{7b}, SR⁷, SOR⁷, S(O)₂R⁷, S(O)₂NR⁷R⁷, CO₂R⁷, C(O)R⁷, C(O)NR⁷R⁷, C₁-C₄-alkyl, C₂-C₄-alkenyl, C₂-C₄-alkynyl, C₁-C₄-haloalkyl, NR⁷—C₀-C₄-alkylene-R^{5c}, O—C₀-C₄-alkylene-R^{5c}, and C₀-C₄-alkylene-R^{5c};
R^{5c} is independently selected from C₃-C₈ cycloalkyl, C₅-C₈ cycloalkenyl, phenyl, 3- to 10-membered heterocycloalkyl, 5- to 10-membered heterocycloalkenyl and 5-, or 6-membered heteroaryl; wherein where R^{5c} is cycloalkyl, cycloalkenyl, heterocycloalkyl or heterocycloalkenyl, R^{5c} is optionally substituted with from 1 to 4 R⁹ groups; and where R^{5c} is phenyl or heteroaryl, R^{5c} is optionally substituted with from 1 to 5 R¹⁰ groups;
   or R³ and R⁴ together with the carbon atoms to which they are attached form a ring selected from: phenyl, C₅-C₇-cycloalkyl, 5- to 7-membered heterocycloalkyl and 5- or 6-membered heteroaryl; wherein where the ring is cycloalkyl or heterocycloalkyl, it is optionally substituted with from 1 to 6 R⁹ groups and where the ring is phenyl or heteroaryl, it is optionally substituted with from 1 to 4 R¹⁰ groups;
   or R⁴ and R⁵ together with the carbon atoms to which they are attached form a ring selected from: phenyl, C₅-C₇-cycloalkyl, 5- to 7-membered heterocycloalkyl and 5- or 6-membered heteroaryl; wherein where the ring is cycloalkyl or heterocycloalkyl, it is optionally substituted with from 1 to 6 R⁹ groups and where the ring is phenyl or heteroaryl, it is optionally substituted with from 1 to 4 R¹⁰ groups;
R⁶ is independently selected from H, halo, C₁-C₆-alkyl, and C₁-C₆-haloalkyl;
R⁷ and R^{7a} are each independently at each occurrence selected from H and C₁-C₄ alkyl;
R^{7b} is independently at each occurrence selected from C₁-C₄ alkyl;
R⁸ is independently at each occurrence selected from H, C₁-C₄ alkyl, C₁-C₄-haloalkyl and C(O)—C₁-C₄-alkyl
R^{8a} is independently at each occurrence selected from H, C₁-C₄ alkyl, C₁-C₄-haloalkyl, C(O)—C₁-C₄-alkyl and phenyl optionally substituted with from 1 to 5 R¹⁰ groups;
   or R^{7a} and R^{8a}, together with the nitrogen atom to which they are attached, form a 5- to 8-membered heterocycloalkyl ring; optionally substituted with 1 to 4 R⁹ groups;
R⁹ is independently at each occurrence selected from ═O, ═S, halo, nitro, cyano, NR⁷R⁸, OR⁷, SR⁷, SOR⁷, S(O)₂R⁷, SO₂NR⁷R⁷, CO₂R⁷, C(O)R⁷, CONR⁷R⁷, C₁-C₄-alkyl, C₂-C₄-alkenyl, C₂-C₄-alkynyl, C₁-C₄-haloalkyl, C₁-C₃-alkylene-OR⁷, and C₁-C₃-alkylene-NR⁷R⁸;
R^{9a} is independently at each occurrence selected from C₃-C₈ cycloalkyl, C₅-C₈ cycloalkenyl, phenyl, 3- to 8-membered heterocycloalkyl, 5- to 8-membered heterocycloalkenyl and 5-, or 6-membered heteroaryl; wherein where R^{9a} is cycloalkyl, cycloalkenyl, heterocycloalkyl or heterocycloalkenyl, R^{9a} is optionally substituted with from 1 to 4 R⁹ groups; and where R^{9a} is phenyl, or heteroaryl, R^{9a} is optionally substituted with from 1 to 5 R¹⁰ groups;
R¹⁰ is independently at each occurrence selected from halo, nitro, cyano, NR⁷R⁸, OR⁷, SR⁷, SOR⁷, S(O)₂R⁷, SO₂NR⁷R⁷, CO₂R⁷, C(O)R⁷, CONR⁷R⁷, C₁-C₄-alkyl, C₂-C₄-alkenyl, C₂-C₄-alkynyl, C₁-C₄-haloalkyl, C₃-C₆-cycloalkyl, C₁-C₃-alkylene-NR⁷R⁸, and C₁-C₃-alkylene-OR⁷; and
wherein any of the aforementioned alkyl, alkylene, alkenyl, or cycloalkyl groups is optionally substituted, where chemically possible, by 1 to 5 substituents which are each independently at each occurrence selected from the group consisting of: C₁-C₄-alkyl, oxo, halo, nitro, cyano, NR^{a}R^{b}, OR^{a}, SR^{a}, CO₂R^{a}, C(O)R^{a}, CONR^{a}R^{a}, S(O)R^{a}, and S(O)₂R^{a}; wherein R^{a} is independently at each occurrence selected from H, and C₁-C₄-alkyl; and R^{b} is independently at each occurrence selected from H, C₁-C₄-alkyl, C(O)—C₁-C₄-alkyl and S(O)₂—C₁-C₄-alkyl.

It may be that R^{2a} is independently at each occurrence selected from H and C₁-C₄ alkyl; and R^{2b} is independently at each occurrence selected from H, C₁-C₄ alkyl, C₁-C₄-haloalkyl, C₀-C₄ alkyl-R^{2c}, C₂-C₄-alkylene-R²⁶, C(O)—C₁-C₄-alkyl, S(O)—C₁-C₄-alkyl, and S(O)₂—C₁-C₄-alkyl.

Compounds according to formula (I) may be able to tautomerize, depending on the identity of the substituent groups, to give alternative tautomeric forms of the compound depicted above. It may be that compounds of formula (I) are a mixture of these tautomeric forms. Said tautomeric forms may be interconverting in any given sample. The relative proportion of tautomeric forms in a given sample will be determined by the position of an equilibrium between those tautomeric forms under the conditions. The position of the equilibrium, and therefore the extent to which each tautomeric form of the compound is present, may be determined by factors including, but not limited to, identity of the substituent groups, temperature, pH, and/or the solvent (where compound of formula (I) is in solution).

In an embodiment, the compound of formula (I) is a compound of formula (II):

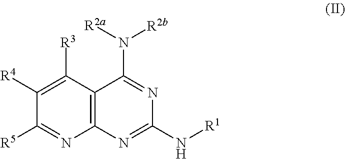

wherein R¹, R^{2a}, R^{2b}, R³, R⁴ and R⁵ are as defined above for formula (I).

In an embodiment, the compound of formula (I) is a compound of formula (III):

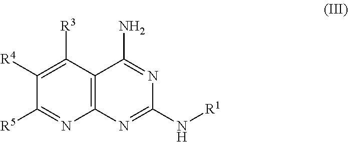

wherein R¹, R³, R⁴ and R⁵ are as defined above for formula (I).

In an embodiment, the compound of formula (I) is a compound of formula (IV):

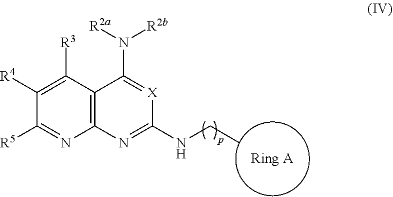

wherein Ring A is independently selected from C₃-C₈ cycloalkyl, C₅-C₈ cycloalkenyl, 3- to 10-membered heterocycloalkyl, and 5- to 10-membered heterocycloalkenyl; wherein Ring A is optionally substituted with C₀-C₅-alkylene-R^{9a} and/or from 1 to 6 R⁹ groups; wherein where Ring A is heterocycloalkyl or heterocycloalkenyl, the heterocycloalkyl or heterocycloalkenyl is optionally fused to a phenyl ring, wherein the phenyl ring is optionally substituted with from 1 to 4 R¹⁰ groups; and p is selected from 0, 1, 2, or 3; and wherein X, R¹, R^{2a}, R^{2b}, R³, R⁴, R⁵, R⁹, R^{9a}, and R¹⁰ are as defined above for formula (I);
optionally wherein X, R¹, R^{2a}, R^{2b}, R³, R⁴ and R⁵ are as defined above for formula (I); and wherein Ring A is independently selected from C₃-C₈ cycloalkyl, C₅-C₈ cycloalkenyl, 3- to 10-membered heterocycloalkyl, and 5- to 10-membered heterocycloalkenyl; wherein Ring A is optionally substituted with from 1 to 4 R⁹ groups; wherein where Ring A is heterocycloalkyl or heterocycloalkenyl, the heterocycloalkyl or heterocycloalkenyl is optionally fused to a phenyl ring, wherein the phenyl ring is optionally substituted with from 1 to 4 R¹⁰ groups; and p is selected from 0, 1, 2, or 3.

In an embodiment, the compound of formula (I) is a compound of formula (V):

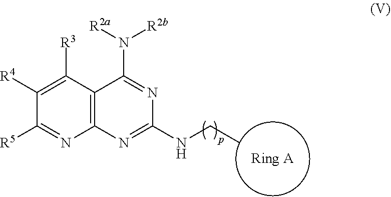

wherein Ring A is independently selected from C₃-C₈ cycloalkyl, C₅-C₈cycloalkenyl, 3- to 10-membered heterocycloalkyl, and 5- to 10-membered heterocycloalkenyl; wherein Ring A is optionally substituted with C₀-C₆-alkylene-R^{9a} and/or from 1 to 6 R⁹ groups; wherein where Ring A is heterocycloalkyl or heterocycloalkenyl, the heterocycloalkyl or heterocycloalkenyl is optionally fused to a phenyl ring, wherein the phenyl ring is optionally substituted with from 1 to 4 R¹⁰ groups; and p is selected from 0, 1, 2, or 3; and wherein X, R¹, R^{2a}, R^{2b}, R³, R⁴, R⁵, R⁹, R^{9a}, and R¹⁰ are as defined above for formula (I);
optionally wherein X, R¹, R^{2a}, R^{2b}, R³, R⁴ and R⁵ are as defined above for formula (I); and wherein Ring A is independently selected from C₃-C₈ cycloalkyl, C₅-C₈cycloalkenyl, 3- to 10-membered heterocycloalkyl, and 5- to 10-membered heterocycloalkenyl; wherein Ring A is optionally substituted with from 1 to 4 R⁹ groups; wherein where Ring A is heterocycloalkyl or heterocycloalkenyl, the heterocycloalkyl or heterocycloalkenyl is optionally fused to a phenyl ring, wherein the phenyl ring is optionally substituted with from 1 to 4 R¹⁰ groups; and p is selected from 0, 1, 2, or 3.

In an embodiment, the compound of formula (I) is a compound of formula (VI):

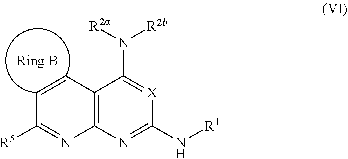

wherein X, R¹, R^{2a}, R^{2b}, and R⁵ are as defined above for formula (I); and wherein Ring B is independently selected from phenyl, C₅-C₇-cycloalkyl, 5- to 7-membered heterocycloalkyl and 5- or 6-membered heteroaryl; wherein where Ring B is cycloalkyl or heterocycloalkyl, it is optionally substituted with from 1 to 6 R⁹ groups and where Ring B is phenyl or heteroaryl, it is optionally substituted with from 1 to 4 R¹⁰ groups;
optionally wherein where Ring B is cycloalkyl or heterocycloalkyl, it is optionally substituted with from 1 to 4 R⁹ groups and where Ring B is phenyl or heteroaryl, it is optionally substituted with from 1 to 4 R¹⁰ groups.

In an embodiment, the compound of formula (I) is a compound of formula (VII):

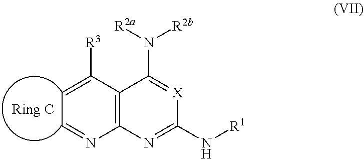

wherein X, R¹, R³, R^{2a}, and R^{2b} are as defined above for formula (I); and wherein Ring C is independently selected from phenyl, C₅-C₇-cycloalkyl, 5- to 7-membered heterocycloalkyl and 5- or 6-membered heteroaryl; wherein where Ring C is cycloalkyl or heterocycloalkyl, it is optionally substituted with from 1 to 6 R⁹ groups and where Ring C is phenyl or heteroaryl, it is optionally substituted with from 1 to 4 R¹⁰ groups;
optionally wherein where Ring C is cycloalkyl or heterocycloalkyl, it is optionally substituted with from 1 to 6 R⁹ groups and where Ring C is phenyl or heteroaryl, it is optionally substituted with from 1 to 4 R¹⁰ groups.

The following embodiments apply to compounds of any of formulae (I)-(VII). These embodiments are independent and interchangeable. Any one embodiment may be combined with any other embodiment, where chemically allowed. In other words, any of the features described in the following embodiments may (where chemically allowable) may be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

R¹ may be C₂-C₆-alkylene-R^{1b}. R^{1b} may be NR^{7a}R^{8a}. R^{7a} may be selected from H and C₁-C₄-alkyl. R^{7a} may be C₁-C₄-alkyl. R^{8a} may be selected from H, C₁-C₄ alkyl and phenyl optionally substituted with from 1 to 5 R¹⁰ groups. R^{8a} may be selected from H and C₁-C₄-alkyl. Rea may be C₁-C₄-alkyl. It may be that R^{7a} and R^{8a}, together with the nitrogen atom to which they are attached, form a 5- to 8-membered heterocycloalkyl ring; optionally substituted with 1 to 4 R⁹ groups.

R¹ may be C₀-C₆-alkylene-R^{1a}. R¹ may be R^{1a}. R¹ may be C₁-C₆-alkylene-R^{1a}.

R^{1a} may be selected from C₃-C₈ cycloalkyl, C₅-C₈ cycloalkenyl, 3- to 10-membered heterocycloalkyl, and 5- to 10-membered heterocycloalkenyl.

R^{1a} may be 3- to 10-membered heterocycloalkyl. R^{1a} may be a 3- to 10-membered heterocycloalkyl group having a nitrogen in the ring system, e.g. pyrrolidine. R^{1a} may be a 3- to 10-membered heterocycloalkyl group having an amine nitrogen in the ring system. Where R^{1a} is a 3- to 10-membered heterocycloalkyl group having an amine nitrogen in the ring system, it may be that it is attached to the rest of the molecule via a carbon atom in the ring system. Where R^{1a} is a 3- to 10-membered heterocycloalkyl group having an amine nitrogen in the ring system, it may be that it is attached to the rest of the molecule via the nitrogen atom in the ring system. R^{1a} may be a monocyclic 3- to 7-membered heterocycloalkyl. R^{1a} may be a piperidine, e.g. a piperidin-4-yl. R^{1a} may be a morpholine. R^{1a} may be a azepane. R^{1a} may be a pyrrolidine. R^{1a} may be a bicyclic 7- to 10-membered heterocycloalkyl. In these embodiments, R^{1a} may be substituted with from 1 to 6 R⁹ groups.

It may be that R¹ is

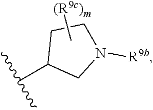

wherein:
R^{9b} is independently selected from H, SOR⁷, S(O)₂R⁷, SO₂NR⁷R⁷, CO₂R⁷, C(O)R⁷, CONR⁷R⁷, C₁-C₄-alkyl, C₂-C₄-alkenyl, C₂-C₄-alkynyl, C₁-C₄-haloalkyl, C₂-C₃-alkylene-OR⁷, and C₂-C₃-alkylene-NR⁷R⁸;
R^{9c} is independently at each occurrence selected from ═O, ═S, halo, nitro, cyano, NR⁷R⁸, OR⁷, SR⁷, SOR⁷, S(O)₂R⁷, SO₂NR⁷R⁷, CO₂R⁷, C(O)R⁷, CONR⁷R⁷, C₁-C₄-alkyl, C₂-C₄-alkenyl, C₂-C₄-alkynyl, C₁-C₄-haloalkyl, C₁-C₃-alkylene-OR⁷, and C₁-C₃-alkylene-NR⁷R⁸; and
m is an integer from 0 to 5.

It may be that R¹ is

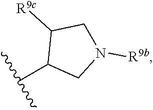

wherein:
R^{9b} is independently selected from H, SOR⁷, S(O)₂R⁷, SO₂NR⁷R⁷, CO₂R⁷, C(O)R⁷, CONR⁷R⁷, C₁-C₄-alkyl, C₂-C₄-alkenyl, C₂-C₄-alkynyl, C₁-C₄-haloalkyl, C₂-C₃-alkylene-OR⁷, and C₂-C₃-alkylene-NR⁷R⁸;
R^{9c} is independently selected from ═O, ═S, halo, nitro, cyano, NR⁷R⁸, OR⁷, SR⁷, SOR⁷, S(O)₂R⁷, SO₂NR⁷R⁷, CO₂R⁷, C(O)R⁷, CONR⁷R⁷, C₁-C₄-alkyl, C₂-C₄-alkenyl, C₂-C₄-alkynyl, C₁-C₄-haloalkyl, C₁-C₃-alkylene-OR⁷, and C₁-C₃-alkylene-NR⁷R⁸.

It may be that R¹ is

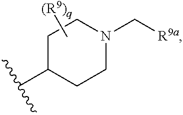

wherein:
q is an integer from 0 to 6; and
R⁹ and R^{9a} are as defined herein.

It may be that R¹ is

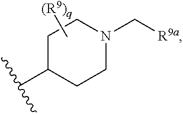

wherein:
q is an integer from 0 to 6;
R⁹ is as defined herein; and
R^{9a} is independently selected from phenyl and 5-, or 6-membered heteroaryl, wherein R⁹¹ is optionally substituted with from 1 to 5 R¹⁰ groups.

It may be that R¹ is

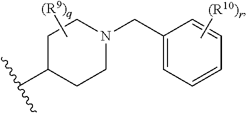

wherein:
q is an integer from 0 to 6;
r is an integer from 0 to 5; and
R⁹ and R¹⁰ are as defined herein.

It may be that R^{9b} is independently selected from H, C₁-C₄-alkyl, and C₁-C₄-haloalkyl. It may be that R^{9b} is independently selected from H and C₁-C₄-alkyl. It may be that R^{9b} is independently H. It may be that R^{9b} is independently methyl.

It may be that m is 0. It may be that m is 1. It may be that m is 2.

It may be that R^{9c} is independently selected from halo, nitro, cyano, NR⁷R⁸, OR⁷, SR⁷, C₁-C₄-alkyl, C₂-C₄-alkenyl, C₂-C₄-alkynyl, C₁-C₄-haloalkyl, C₁-C₃-alkylene-OR⁷, and C₁-C₃-alkylene-NR⁷R⁸.

It may be that R^{9c} is independently selected from NR⁷R⁸, OR⁷, C₁-C₄-alkyl, C₁-C₄-haloalkyl, C₁-C₃-alkylene-OR⁷, and C₁-C₃-alkylene-NR⁷R⁸. It may be that R^{9c} is independently selected from OR⁷ and C₁-C₄-alkyl. It may be that R^{9c} is methyl. It may be that R^{9c} is OH.

R^{1a} may be a 3- to 10-membered heterocycloalkyl wherein the heterocycloalkyl is fused to a phenyl ring. R^{1a} may be a 3- to 6-membered heterocycloalkyl wherein the heterocycloalkyl is fused to a phenyl ring. R^{1a} may be a 5-membered heterocycloalkyl wherein the heterocycloalkyl is fused to a phenyl ring. It may be that R^{1a} is bicyclic when the heterocycloalkyl is fused to a phenyl ring. It may be that the phenyl ring is substituted with from 1 to 4 R¹⁰ groups.

R^{1a} may be a 5- to 10-membered heterocycloalkenyl wherein the heterocycloalkenyl is fused to a phenyl ring. R^{1a} may be a 5- or 6-membered heterocycloalkenyl wherein the heterocycloalkenyl is fused to a phenyl ring. It may be that R^{1a} is bicyclic when the heterocycloalkenyl is fused to a phenyl ring. It may be that the phenyl ring is substituted with from 1 to 4 R¹⁰ groups.

It may be that R^{1a} is selected from a C₃-C₆-cycloalkyl group or a 3- to 10-membered heterocycloalkyl group that does not contain a nitrogen in the ring system and R^{1a} is substituted with at least one NR⁷R⁸ group.

It may be that R¹ contains 1, 2, or 3 nitrogen atoms. It may be that R¹ contains a single nitrogen atom. It may be that R¹ contains 2 nitrogen atoms. Where R¹ contains 2 or 3 nitrogen atoms, at least one of the nitrogen atoms is an amine nitrogen. It may be that R¹ comprises 1, 2, or 3 amine nitrogen atoms, optionally a single amine nitrogen.

Ring A may be 3- to 10-membered heterocycloalkyl. Ring A may be a 3- to 10-membered heterocycloalkyl group having a nitrogen in the ring system, e.g. pyrrolidine. Ring A may be a 3- to 10-membered heterocycloalkyl group having an amine nitrogen in the ring system. Where Ring A is a 3- to 10-membered heterocycloalkyl group having an amine nitrogen in the ring system, it may be that it is attached to the rest of the molecule via a carbon atom in the ring system. Where Ring A is a 3- to 10-membered heterocycloalkyl group having an amine nitrogen in the ring system, it may be that it is attached to the rest of the molecule via the nitrogen atom in the ring system. Ring A may be a monocyclic 3- to 7-membered heterocycloalkyl. Ring A may be a piperidine, e.g. a piperidin-4-yl. Ring A may be a morpholine. Ring A may be a azepane. Ring A may be a pyrrolidine. Ring A may be a bicyclic 7- to 10-membered heterocycloalkyl. p may be 0. p may be 1. p may be 2. p may be 3.

It may be that Ring A is selected from a C₃-C₆-cycloalkyl group or a 3- to 10-membered heterocycloalkyl group that does not contain a nitrogen in the ring system and Ring A is substituted with at least one NR⁷R⁸ group.

Ring A may be a 3- to 10-membered heterocycloalkyl wherein the heterocycloalkyl is fused to a phenyl ring. Ring A may be a 3- to 6-membered heterocycloalkyl wherein the heterocycloalkyl is fused to a phenyl ring. Ring A may be a 5-membered heterocycloalkyl wherein the heterocycloalkyl is fused to a phenyl ring. Ring A fused to a phenyl ring may be a bicyclic ring system. It may be that the phenyl ring is substituted with from 1 to 4 R¹⁰ groups.

Ring A may be a 5- to 10-membered heterocycloalkenyl wherein the heterocycloalkenyl is fused to a phenyl ring. Ring A may be a 5- or 6-membered heterocycloalkenyl wherein the heterocycloalkenyl is fused to a phenyl ring. Ring A fused to a phenyl ring may be a bicyclic ring system. It may be that the phenyl ring is substituted with from 1 to 4 R¹⁰ groups.

It may be that R¹ comprises at least one amine nitrogen. An amine nitrogen is a nitrogen in an amine group. For the avoidance of doubt, the term “amine” as used herein encompasses primary amines, e.g., methylamine; secondary amines, e.g., dimethylamine; tertiary amines, e.g., trimethylamine; cyclic amines, e.g., piperidine. For the avoidance of doubt, the term “amine” as used herein excludes amides (including lactams) and sulfonamides (including cyclic sulfonamides).

R^{2a} may be independently selected from H and C₁-C₂ alkyl. R^{2a} may be H.

R^{2b} may be independently selected from H, C₁-C₄ alkyl, C₁-C₄-haloalkyl, C₀-C₄ alkyl-R^{2c} and C₂-C₄-alkylene-R^{2d}. R^{2b} may be independently selected from H, C₁-C₄ alkyl, and C₀-C₄ alkyl-R^{2c}. R^{2b} may be independently selected from H, and C₁-C₄ alkyl. R^{2b} may be independently selected from H, and C₁-C₂ alkyl. R^{2b} may be H.

R^{2b} may be C₂-C₄alkylene-R^{2d}. R^{2d} may be NR⁷R⁸. R^{2d} may be OR⁷.

It may be that at least one of R^{2a} and R^{2b} is H. It may be that R^{2a} is H and R^{2b} is selected from H and methyl. It may be that R^{2a} is H and R^{2b} is methyl. It may be that R^{2a} and R^{2b} are each H.

X may be N. X may be CR⁶, e.g. CH.

R³ may be H. R³ may be C₁-C₄-alkyl, e.g. methyl.

It may be that R⁴ is independently selected from H, halo, cyano, OR⁷, SR⁷, SOR⁷, S(O)₂R⁷, S(O)₂NR⁷R⁷, CO₂R⁷, C(O)R⁷, C(O)NR⁷R⁷, C₁-C₄-alkyl, C₂-C₄-alkenyl, C₂-C₄-alkynyl, C₁-C₄-haloalkyl, O—C₀-C₄-alkylene-R^{4c}, and C₀-C₄alkylene-R^{4c}.

R⁴ may be H. R⁴ may be C₁-C₄-alkyl, e.g. methyl.

It may be that R⁴ is C₀-C₄-alkylene-R^{4c}, i.e., R⁴ is R^{4c}.

It may be that R^{4c} is independently selected from C₃-C₈ cycloalkyl, C₅-C₈ cycloalkenyl, 3- to 10-membered heterocycloalkyl, and 5- to 10-membered heterocycloalkenyl; wherein R^{4c} is optionally substituted with from 1 to 4 R⁹ groups.

R³ and R⁴ together with the carbon atoms to which they are attached may form a ring selected from: C₅-C₇-cycloalkyl and 5- to 7-membered heterocycloalkyl; optionally wherein the ring is substituted with from 1 to 6 R⁹ groups. R³ and R⁴ together with the carbon atoms to which they are attached may form a ring selected from: C₅-C₇-cycloalkyl and 5- to 7-membered heterocycloalkyl; optionally wherein the ring is substituted with from 1 to 4 R⁹ groups.

R³ and R⁴ together with the carbon atoms to which they are attached may form a C₅-C₇-cycloalkyl ring; optionally wherein the ring is substituted with from 1 to 6 R⁹ groups. R³ and R⁴ together with the carbon atoms to which they are attached may form a C₅-C₇-cycloalkyl ring; optionally wherein the ring is substituted with from 1 to 4 R⁹ groups.

R³ and R⁴ together with the carbon atoms to which they are attached may form a C₅-C₇-cycloalkyl ring; optionally wherein the ring is substituted with from 1 to 6 R⁹ groups. R³ and R⁴ together with the carbon atoms to which they are attached may form a C₅-C₇-cycloalkyl ring; optionally wherein the ring is substituted with from 1 to 4 R⁹ groups.

It may be that R⁵ is independently selected from H, halo, cyano, C₁-C₄-alkylene-NR⁷R⁸, C₁-C₄-alkylene-OR⁷, SR⁷, SOR⁷, S(O)₂R⁷, S(O)₂NR⁷R⁷, CO₂R⁷, C(O)R⁷, C(O)NR⁷R⁷, C₁-C₄-alkyl, C₂-C₄-alkenyl, C₂-C₄-alkynyl, C₁-C₄-haloalkyl, O—C₀-C₄alkylene-R^{5c}, and C₀-C₄-alkylene-R^{5c}.

R⁵ may be H. R⁵ may be C₁-C₄-alkyl, e.g. methyl.

R⁵ may be R^{5c}. R^{5c} may be phenyl optionally substituted with from 1 to 5 R¹⁰ groups. R^{5c} may be 5-, or 6-membered heteroaryl optionally substituted with from 1 to 5 R¹⁰ groups. R^{5c} may be 6-membered heteroaryl optionally substituted with from 1 to 5 R¹⁰ groups.

R⁴ and R⁵ together with the carbon atoms to which they are attached may form a ring selected from: C₅-C₇-cycloalkyl and 5- to 7-membered heterocycloalkyl; optionally wherein the ring is substituted with from 1 to 6 R⁹ groups. R⁴ and R⁵ together with the carbon atoms to which they are attached may form a ring selected from: C₅-C₇-cycloalkyl and 5- to 7-membered heterocycloalkyl; optionally wherein the ring is substituted with from 1 to 4 R⁹ groups.

R⁴ and R⁵ together with the carbon atoms to which they are attached may form a C₅-C₇-cycloalkyl ring; optionally wherein the ring is substituted with from 1 to 6 R⁹ groups. R⁴ and R⁵ together with the carbon atoms to which they are attached may form a C₅-C₇-cycloalkyl ring; optionally wherein the ring is substituted with from 1 to 4 R⁹ groups.

R⁴ and R⁵ together with the carbon atoms to which they are attached may form a C₅-C₇-cycloalkyl ring; optionally wherein the ring is substituted with from 1 to 6 R⁹ groups. R⁴ and R⁵ together with the carbon atoms to which they are attached may form a C₅-cycloalkyl ring; optionally wherein the ring is substituted with from 1 to 4 R⁹ groups.

Ring B may be independently selected from C₅-C₇-cycloalkyl, and 5- to 7-membered heterocycloalkyl; optionally wherein Ring B is substituted with from 1 to 6 R⁹ groups. Ring B may be independently selected from C₅-C₇-cycloalkyl, and 5- to 7-membered heterocycloalkyl; optionally wherein Ring B is substituted with from 1 to 4 R⁹ groups.

Ring B may be C₅-C₇-cycloalkyl; optionally substituted with from 1 to 6 R⁹ groups. Ring B may be C₅-cycloalkyl; optionally substituted with from 1 to 6 R⁹ groups. Ring B may be C₅-C₇-cycloalkyl; optionally substituted with from 1 to 6 R⁹ groups. Ring B may be C₅-cycloalkyl; optionally substituted with from 1 to 4 R⁹ groups.

Ring C may be independently selected from C₅-C₇-cycloalkyl, and 5- to 7-membered heterocycloalkyl; optionally wherein Ring C is substituted with from 1 to 6 R⁹ groups. Ring C may be independently selected from C₅-C₇-cycloalkyl, and 5- to 7-membered heterocycloalkyl; optionally wherein Ring C is substituted with from 1 to 4 R⁹ groups.

Ring C may be C₅-C₇-cycloalkyl; optionally substituted with from 1 to 6 R⁹ groups. Ring C may be C₅-cycloalkyl; optionally substituted with from 1 to 6 R⁹ groups. Ring C may be C₅-C₇-cycloalkyl; optionally substituted with from 1 to 4 R⁹ groups. Ring C may be C₅-cycloalkyl; optionally substituted with from 1 to 4 R⁹ groups.

R⁹ may independently at each occurrence be selected from ═O, ═S, halo, nitro, cyano, NR⁷R⁸, OR⁷, SR⁷, SOR⁷, S(O)₂R⁷, SO₂NR⁷R⁷, CO₂R⁷, C(O)R⁷, CONR⁷R⁷, C₁-C₄-alkyl, C₂-C₄-alkenyl, C₂-C₄-alkynyl, C₁-C₄-haloalkyl, and C₁-C₃-alkylene-NR⁷R⁸.

R⁹ may independently at each occurrence be selected from halo, NR⁷R⁸, OR⁷, CO₂R⁷, CONR⁷R⁷, C₁-C₄-alkyl, C₁-C₃-alkylene-OR⁷, and C₁-C₃-alkylene-NR⁷R⁸.

R⁹ may independently at each occurrence be selected from F, Cl, Br, NR⁷R⁸, OR⁷, CO₂R⁷, CONR⁷R⁷, C₁-C₄-alkyl, C₁-C₃-alkylene-OR⁷, and C₁-C₃-alkylene-NR⁷R⁸.

R⁹ may independently at each occurrence be selected from F, NR⁷R⁸, OR⁷, CO₂R⁷, CONR⁷R⁷, C₁-C₄-alkyl, C₁-C₃-alkylene-OR⁷, and C₁-C₃-alkylene-NR⁷R⁸.

R⁹ may independently at each occurrence be selected from F, NR⁷R⁸, OR⁷, CO₂R⁷, CONR⁷R⁷, and C₁-C₄-alkyl.

R⁹ may independently at each occurrence be selected from OH and C₁-C₄-alkyl.

Rea may independently at each occurrence be selected from C₃-C₈ cycloalkyl, phenyl, 3- to 8-membered heterocycloalkyl, and 5-, or 6-membered heteroaryl; wherein where Rea is cycloalkyl, or heterocycloalkyl, R^{9a} is optionally substituted with from 1 to 4 R⁹ groups; and where R^{9a} is phenyl, or heteroaryl, R^{9a} is optionally substituted with from 1 to 5 R¹⁰ groups.

R^{9a} may independently at each occurrence be phenyl, optionally substituted with from 1 to 5 R¹⁰ groups.

R¹⁰ may independently at each occurrence be selected from halo, cyano, OR⁷, C₁-C₄-alkyl, C₁-C₄-haloalkyl, C₁-C₃-alkylene-NR⁷R⁸, and C₁-C₃-alkylene-OR⁷.

R¹⁰ may independently at each occurrence be selected from halo, cyano, OR⁷, C₁-C₄-alkyl, and C₁-C₄-haloalkyl.

R¹⁰ may independently at each occurrence be selected from F, Cl, Br, cyano, OR⁷, C₁-C₄-alkyl, and C₁-C₄-haloalkyl.

R¹⁰ may independently at each occurrence be selected from F, cyano, OR⁷, C₁-C₄-alkyl, and C₁-C₄-haloalkyl.

In an embodiment, any of the alkyl, alkylene, alkenyl or cycloalkyl groups are optionally substituted, where chemically possible, by 1 to 5 substituents which are each independently at each occurrence selected from the group consisting of: oxo, fluoro, NR^{a}R^{b}, OR^{a}, and S(O)₂R^{a}; wherein R^{a} is independently at each occurrence selected from H, and C₁-C₄-alkyl; and R^{b} is independently at each occurrence selected from H, C₁-C₄-alkyl, C(O)—C₁-C₄-alkyl and S(O)₂—C₁-C₄-alkyl.

It may be that R¹ is a 3- to 10-membered heterocycloalkyl group having a nitrogen in the ring system, e.g. pyrrolidine, optionally substituted with C₀-C₆-alkylene-R^{9a} and/or from 1 to 6 R⁹ groups; and R⁴ and R⁵ together with the carbon atoms to which they are attached form a C₅-C₇-cycloalkyl ring; optionally wherein the ring is substituted with from 1 to 6 R⁹ groups.

It may be that R¹ is a 3- to 10-membered heterocycloalkyl group having a nitrogen in the ring system, e.g. pyrrolidine, optionally substituted with C₀-C₆-alkylene-R^{9a} and/or from 1 to 6 R⁹ groups; and R⁴ and R⁵ together with the carbon atoms to which they are attached form a C₅-cycloalkyl ring; optionally wherein the ring is substituted with from 1 to 6 R⁹ groups.

It may be that R¹ is a 3- to 10-membered heterocycloalkyl group having a nitrogen in the ring system, e.g. pyrrolidine, optionally substituted with C₀-C₆-alkylene-R^{9a} and/or from 1 to 6 R⁹ groups; and Ring C is a C₅-C₇-cycloalkyl ring; optionally substituted with from 1 to 6 R⁹ groups.

It may be that R¹ is a 3- to 10-membered heterocycloalkyl group having a nitrogen in the ring system, e.g. pyrrolidine, optionally substituted with C₀-C₆-alkylene-R^{9a} and/or from 1 to 6 R⁹ groups; and Ring C is C₅-cycloalkyl; optionally substituted with from 1 to 6 R⁹ groups.

In an embodiment, the compound of formula (I) is selected from:

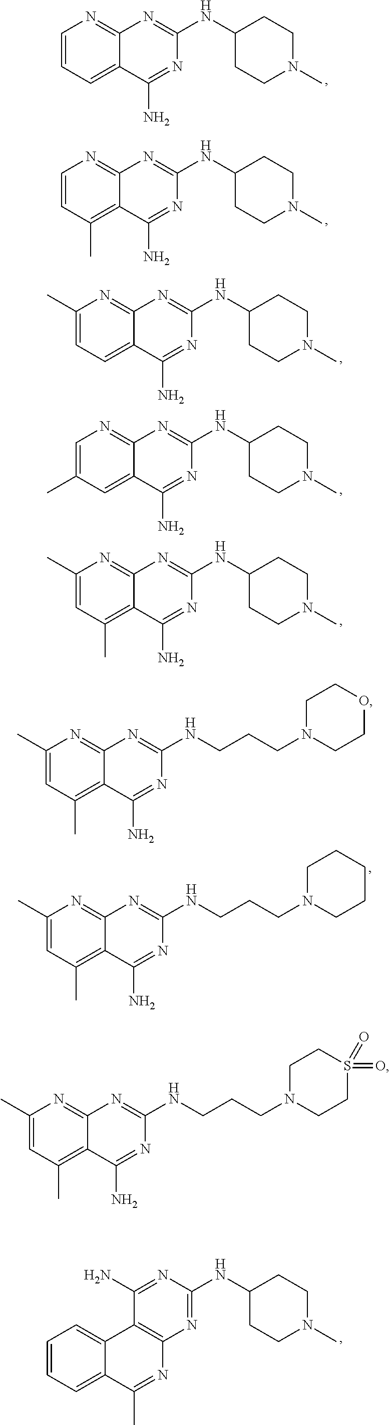

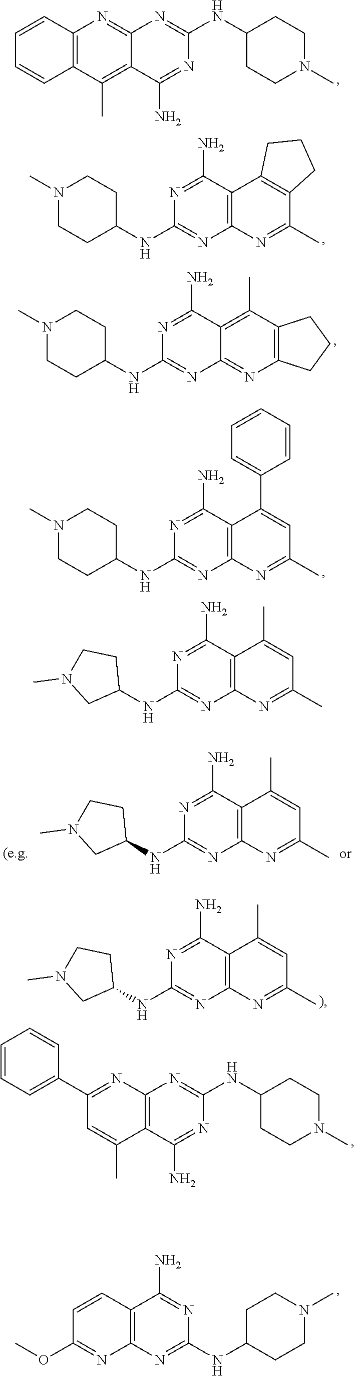

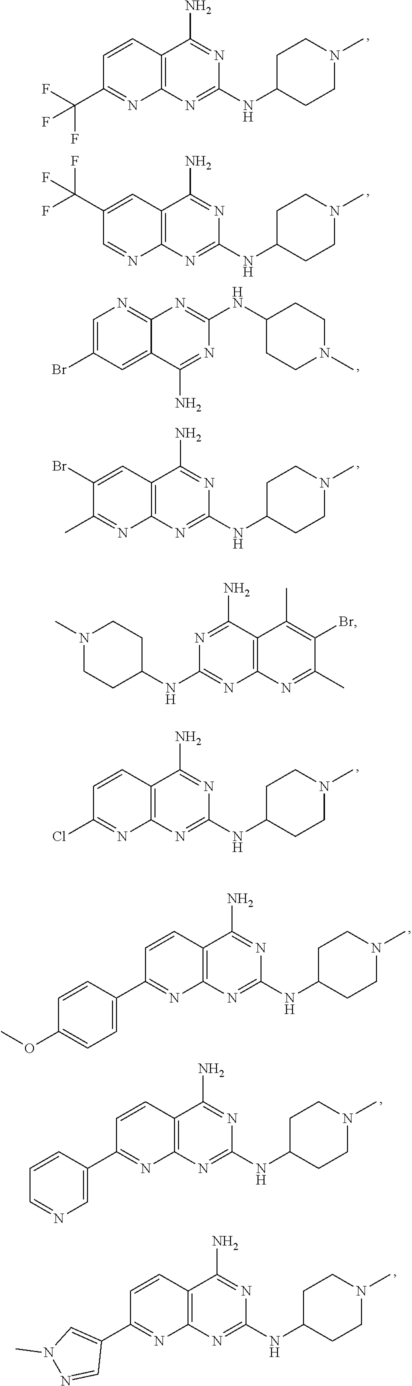

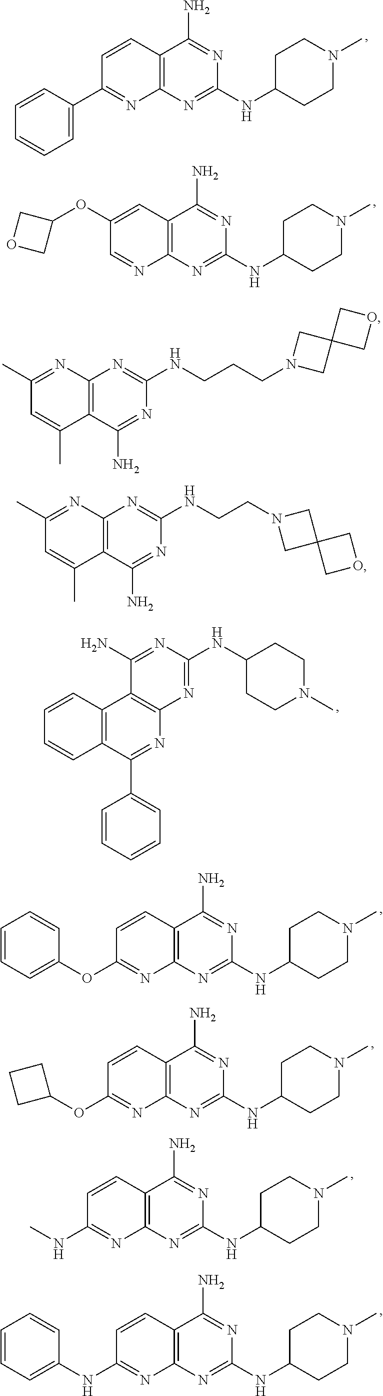

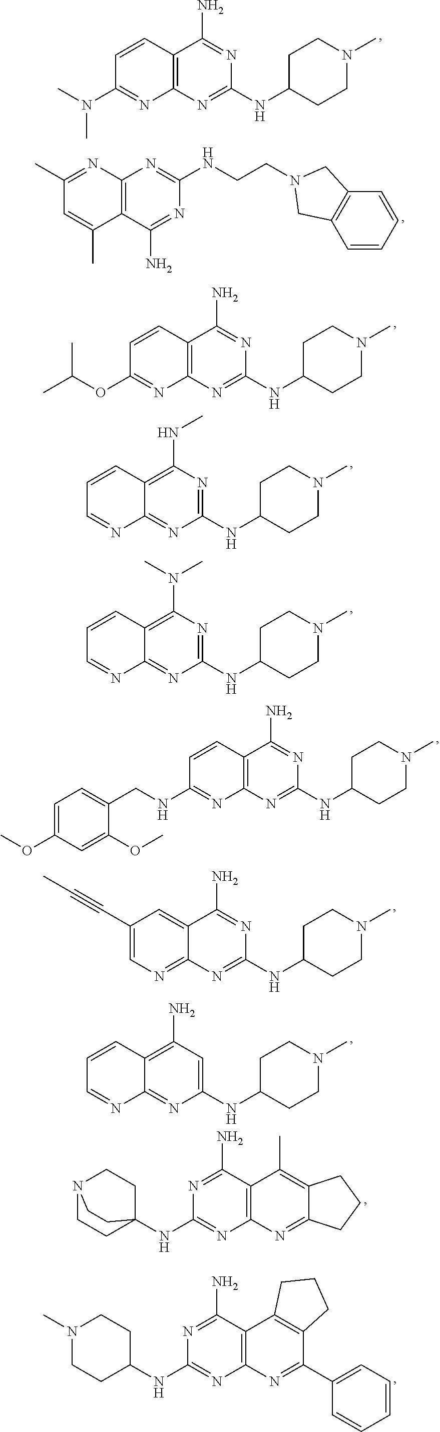

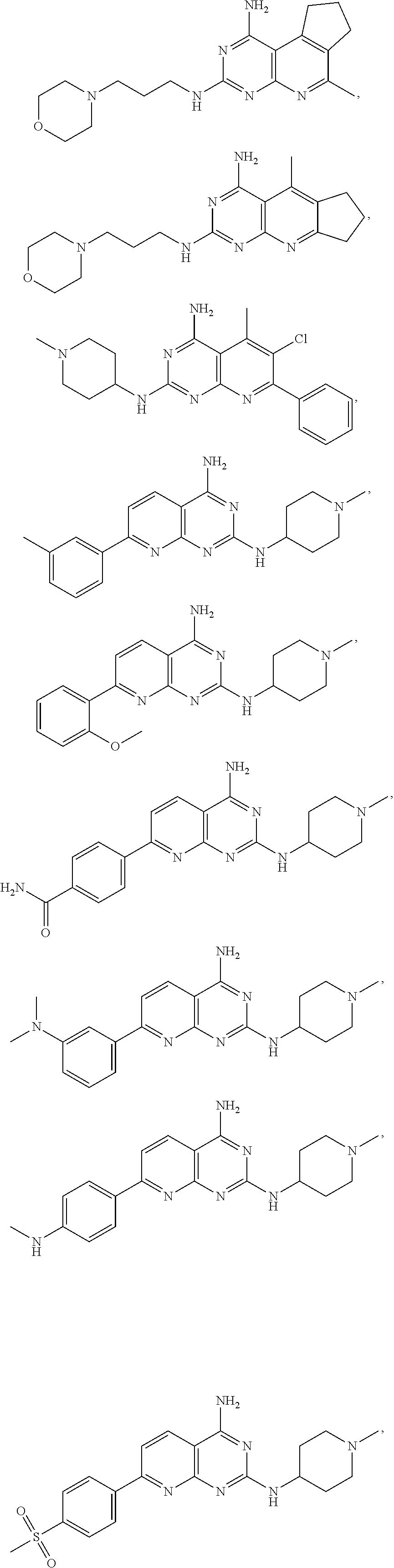

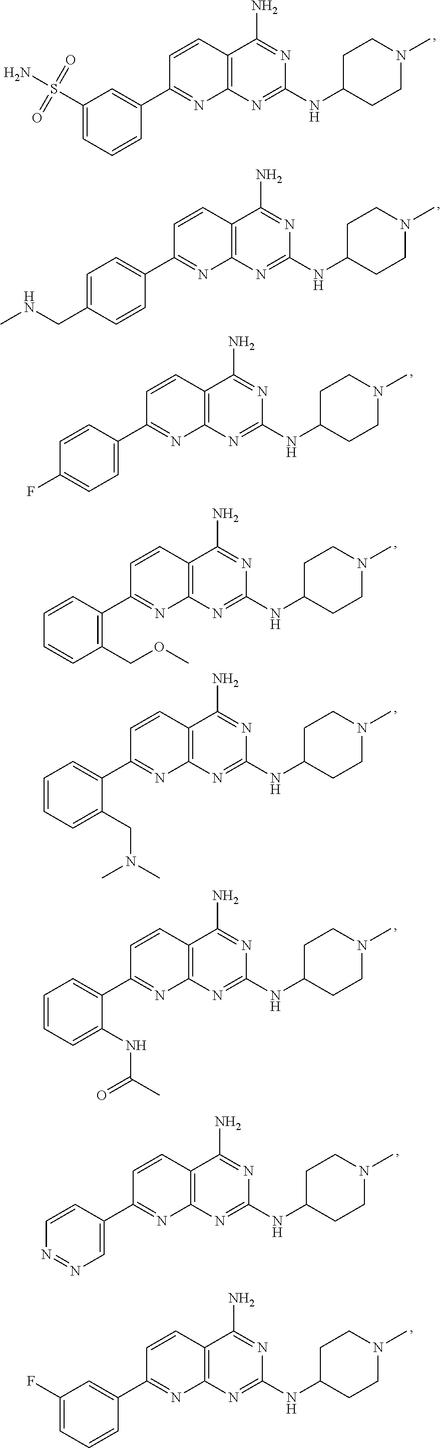

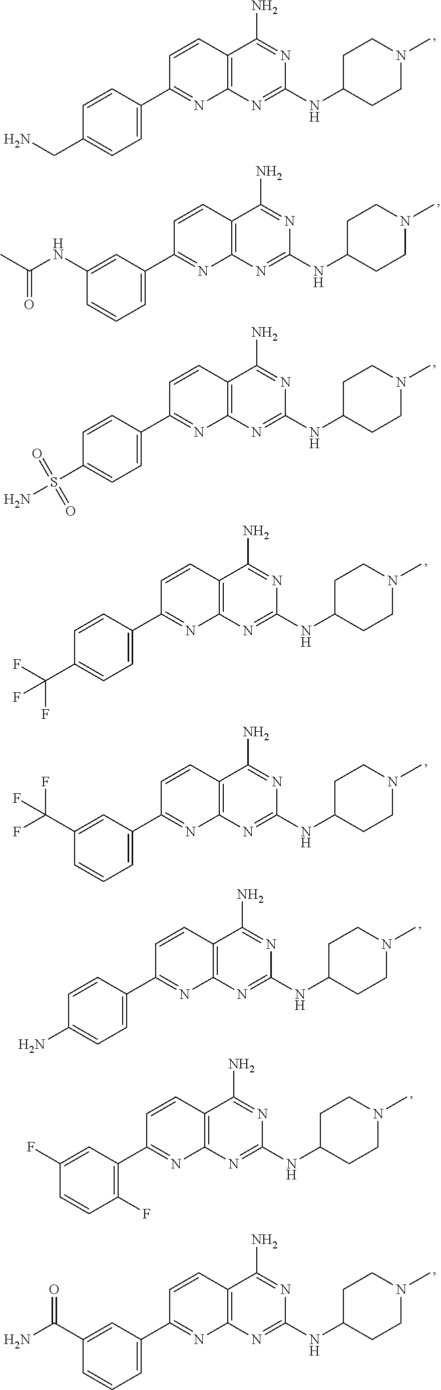

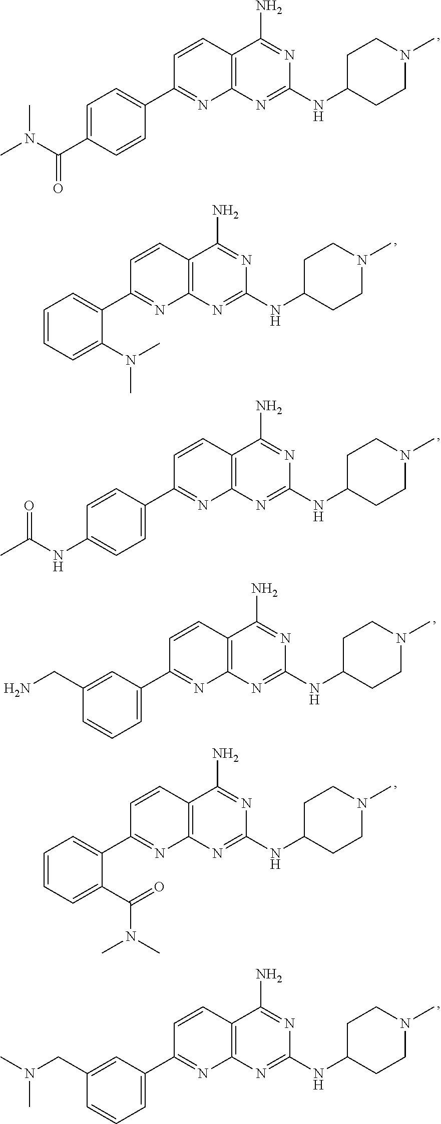

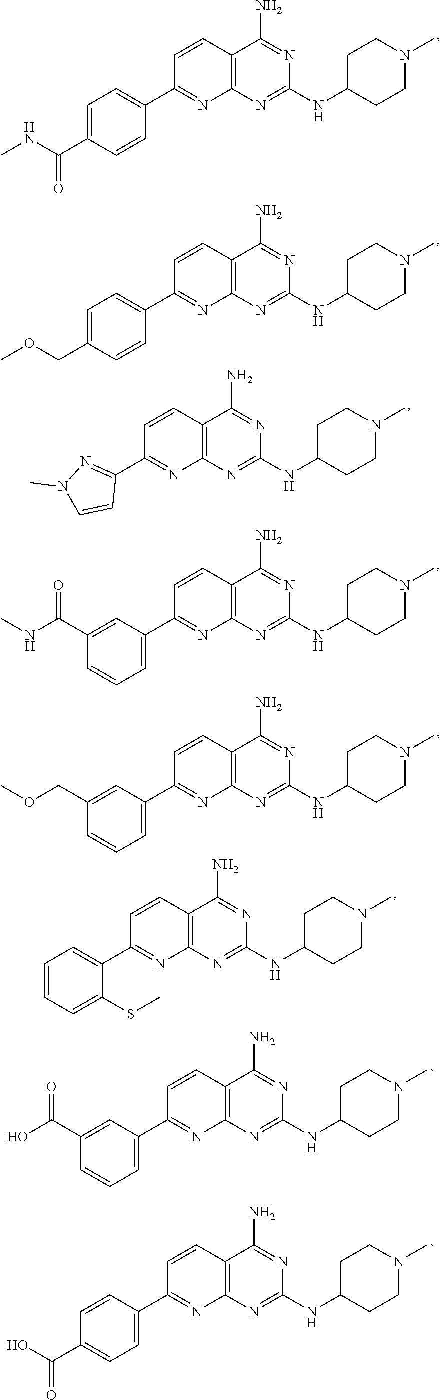

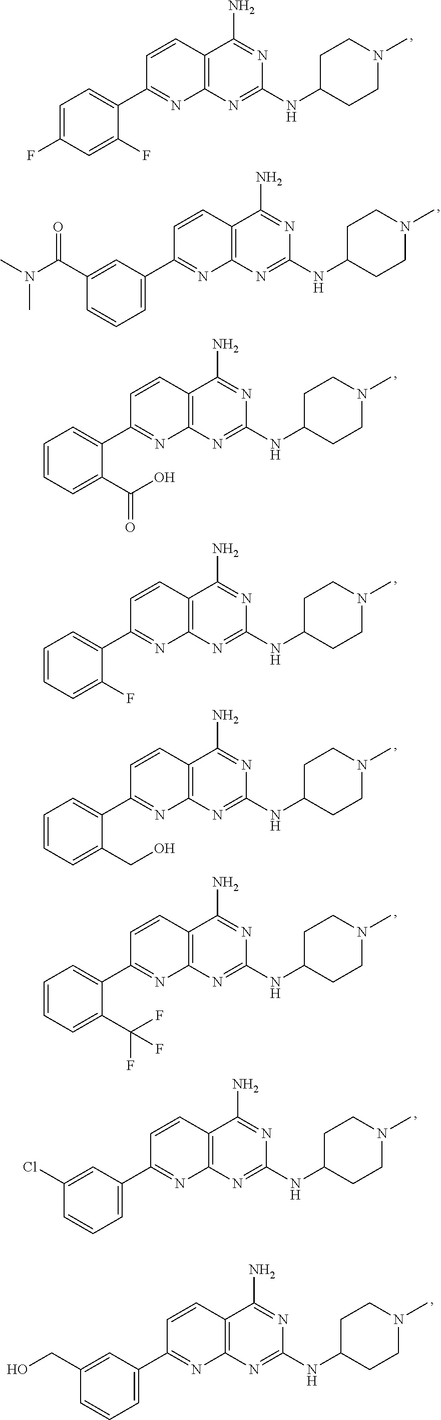

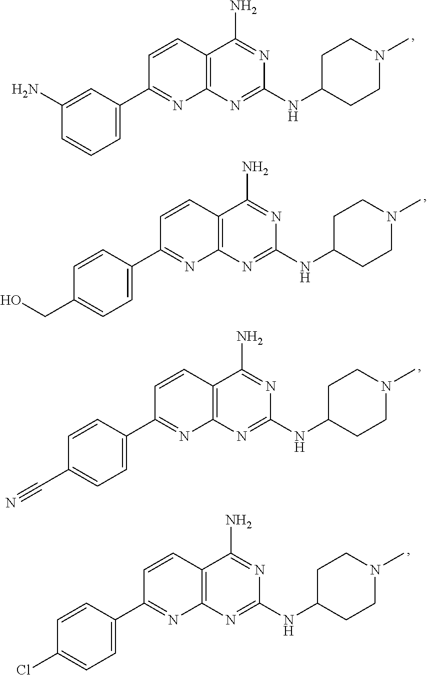 [SMILES: CN1CCC(Nc2nc(N)c3ccc(-c4cccc(N)c4)nc3n2)CC1 ; CN1CCC(Nc2nc(N)c3ccc(-c4ccc(CO)cc4)nc3n2)CC1 ; CN1CCC(Nc2nc(N)c3ccc(-c4ccc(C#N)cc4)nc3n2)CC1 ; CN1CCC(Nc2nc(N)c3ccc(-c4ccc(Cl)cc4)nc3n2)CC1]

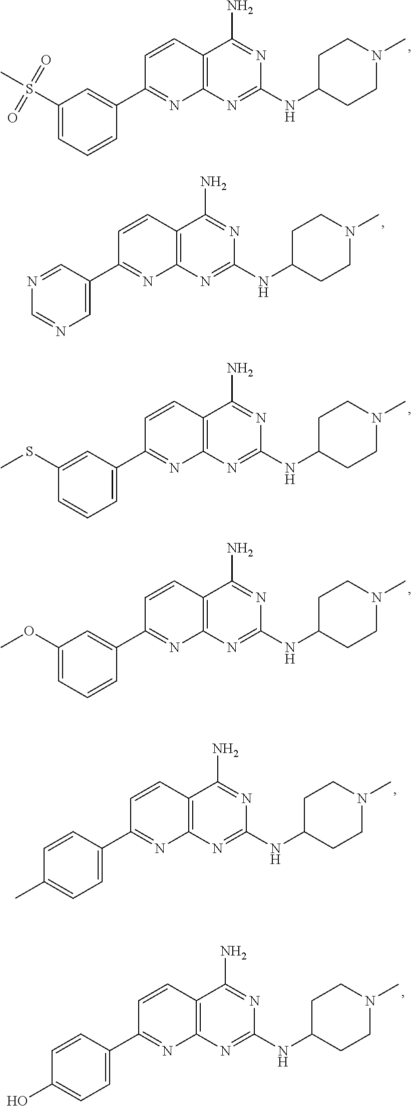

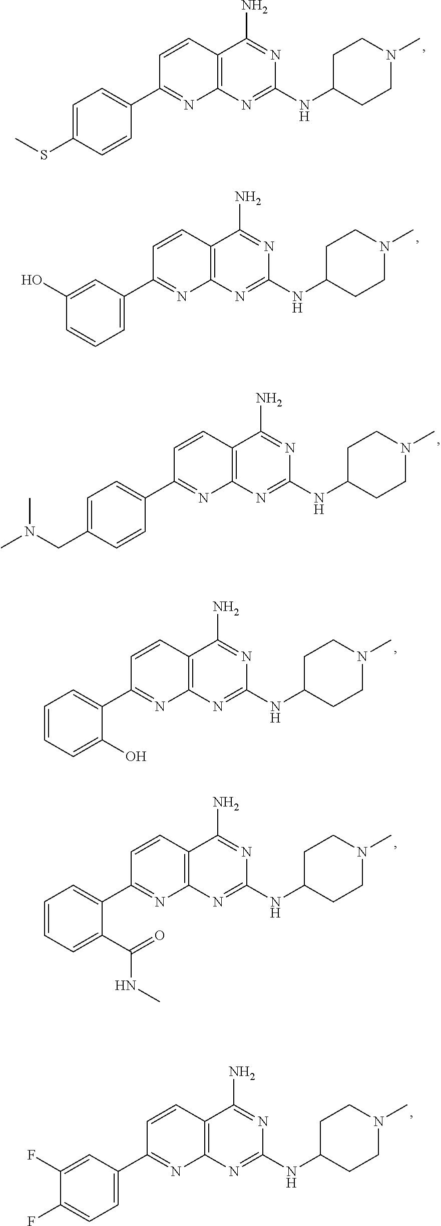

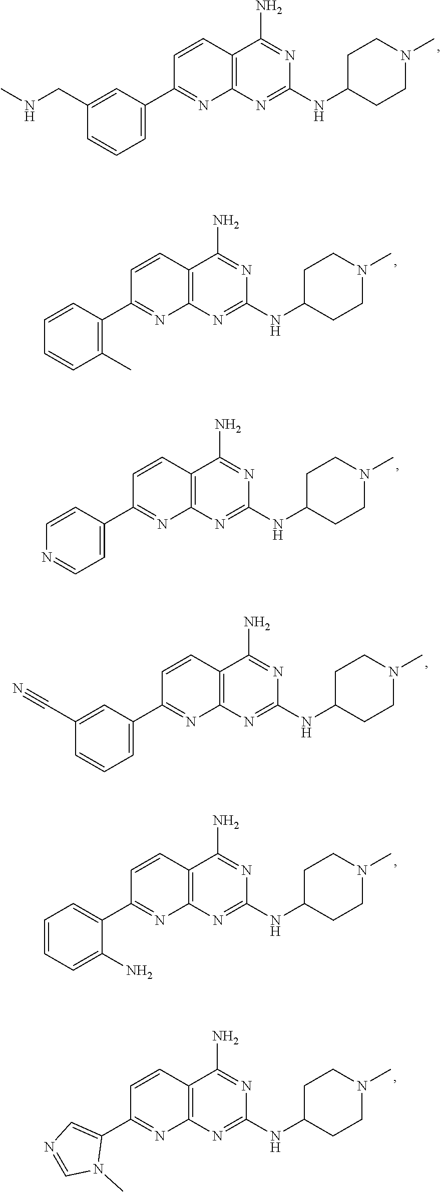

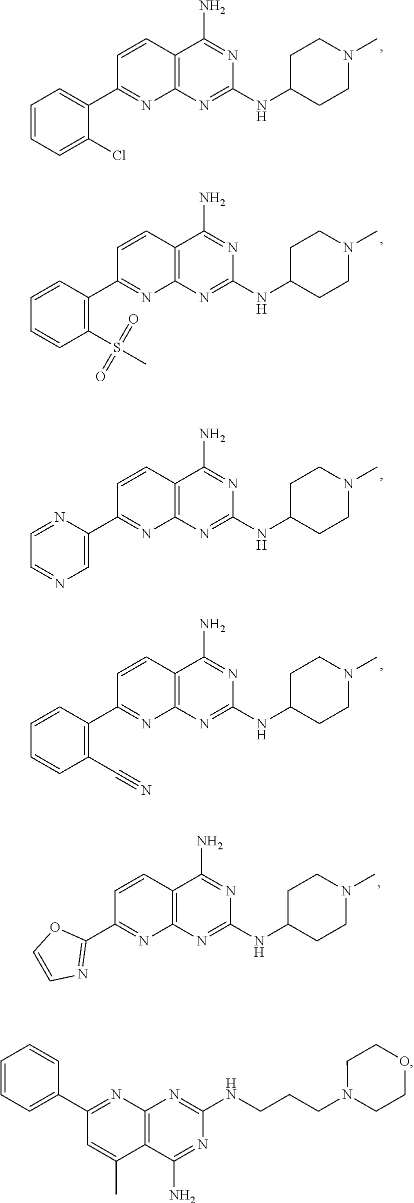 [SMILES: CN1CCC(Nc2nc(N)c3ccc(-c4ccccc4Cl)nc3n2)CC1 ; CN1CCC(Nc2nc(N)c3ccc(-c4ccccc4S(C)(=O)=O)nc3n2)CC1 ; CN1CCC(Nc2nc(N)c3ccc(-c4cnccn4)nc3n2)CC1 ; CN1CCC(Nc2nc(N)c3ccc(-c4ccccc4C#N)nc3n2)CC1 ; CN1CCC(Nc2nc(N)c3ccc(-c4ncco4)nc3n2)CC1 ; Cc1cc(-c2ccccc2)nc2nc(NCCCN3CCOCC3)nc(N)c12]

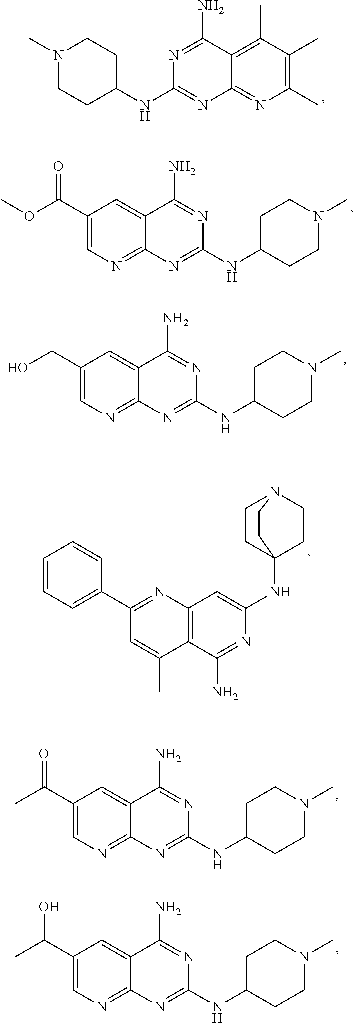

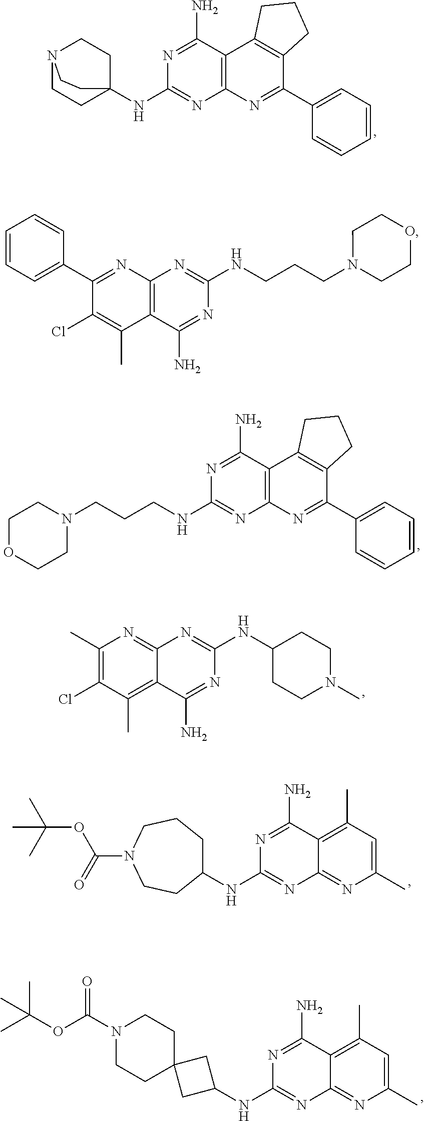

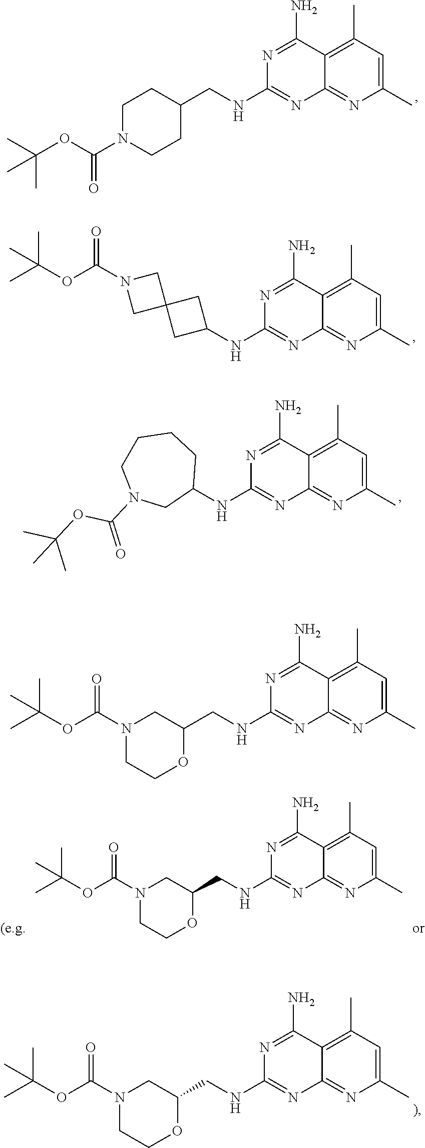

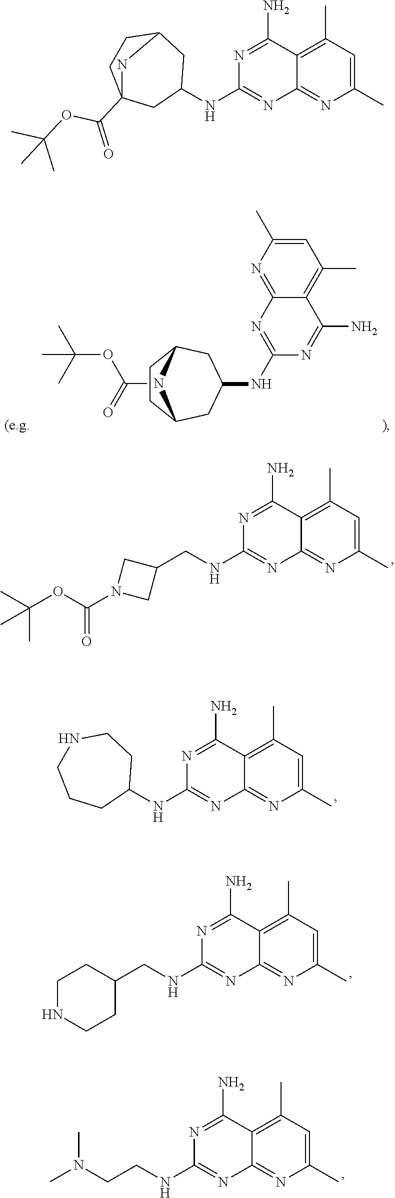

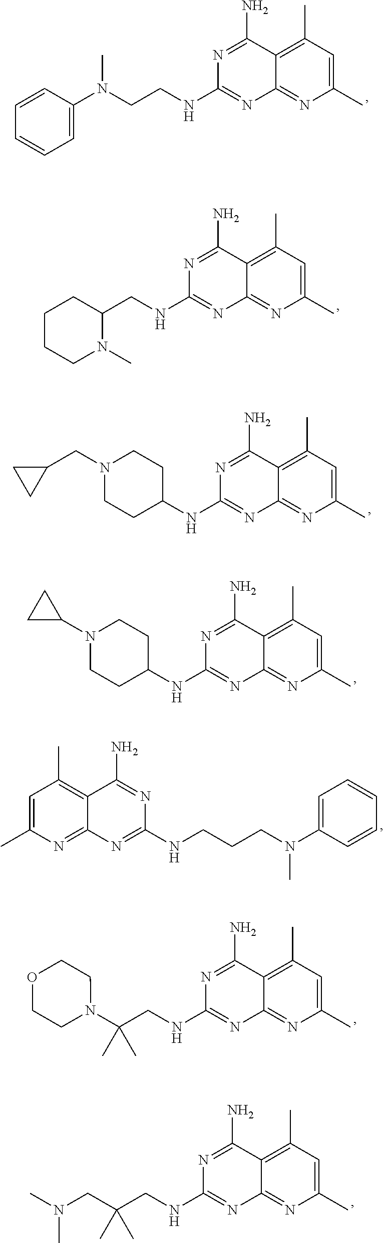

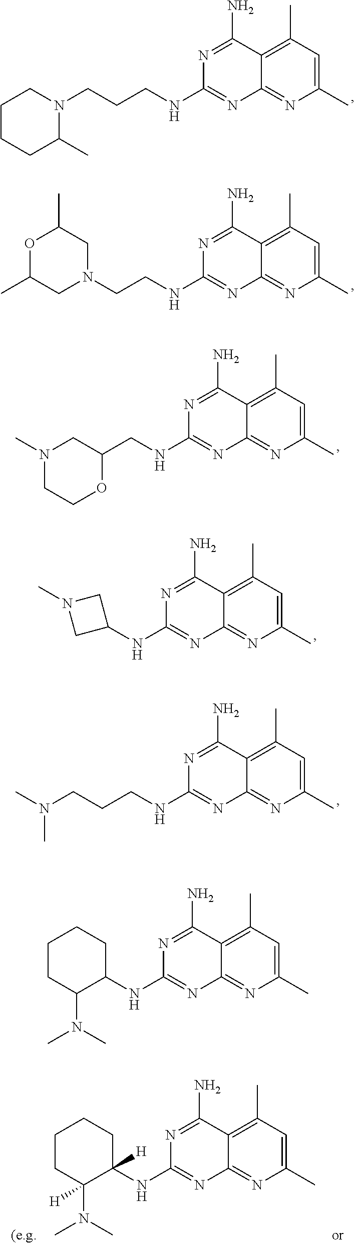

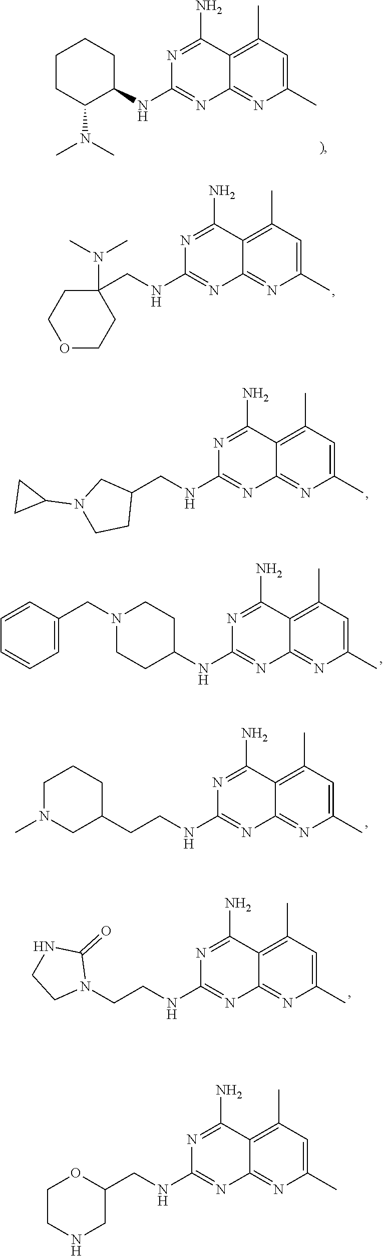

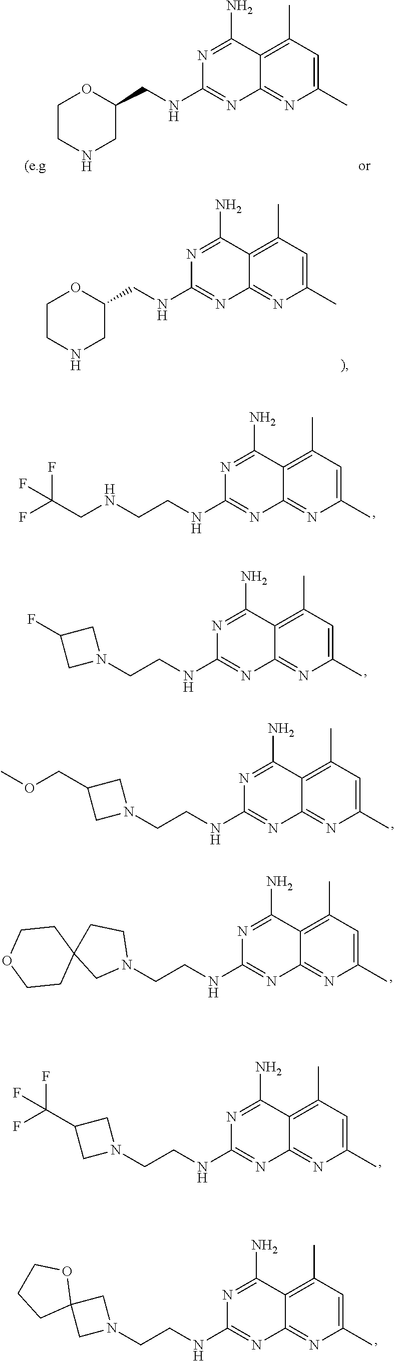

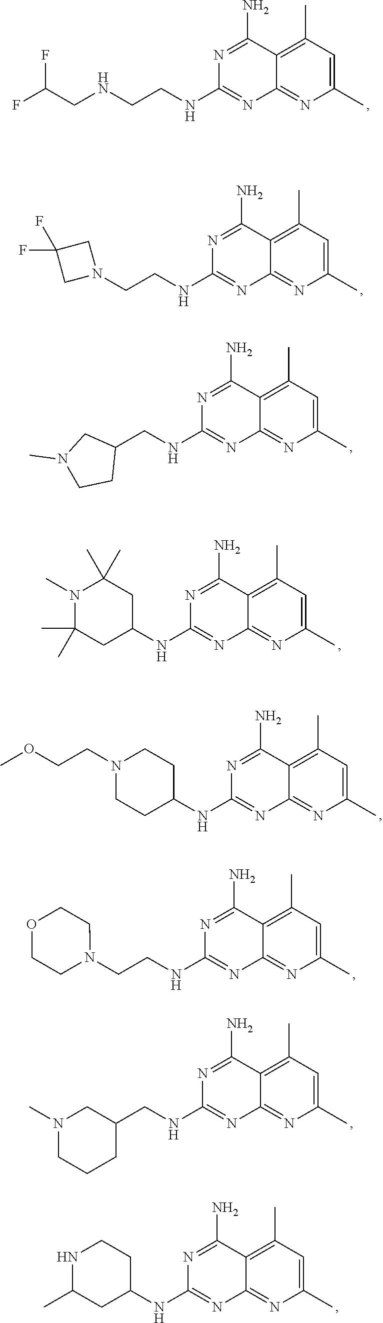

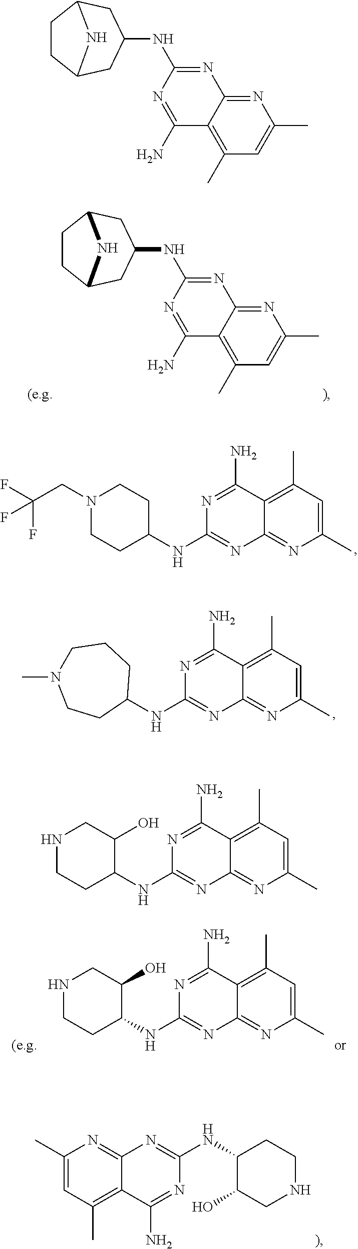

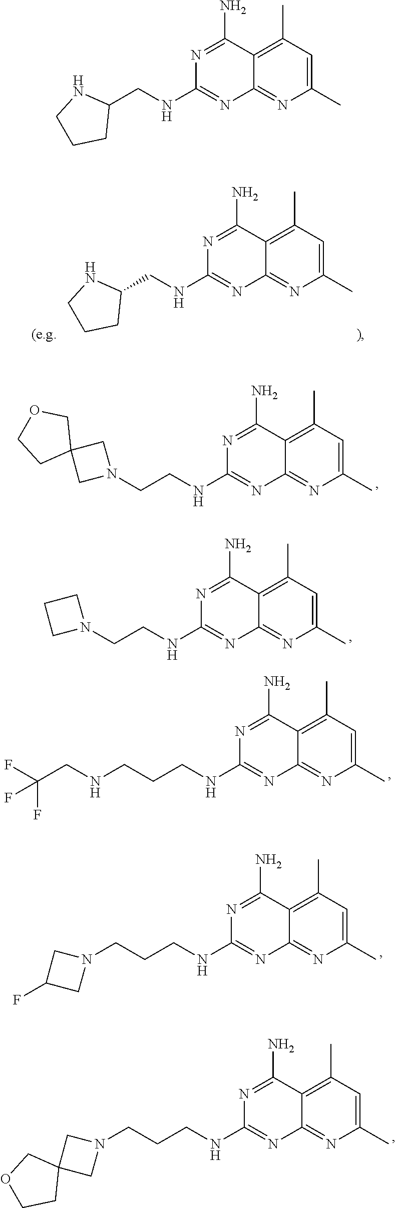

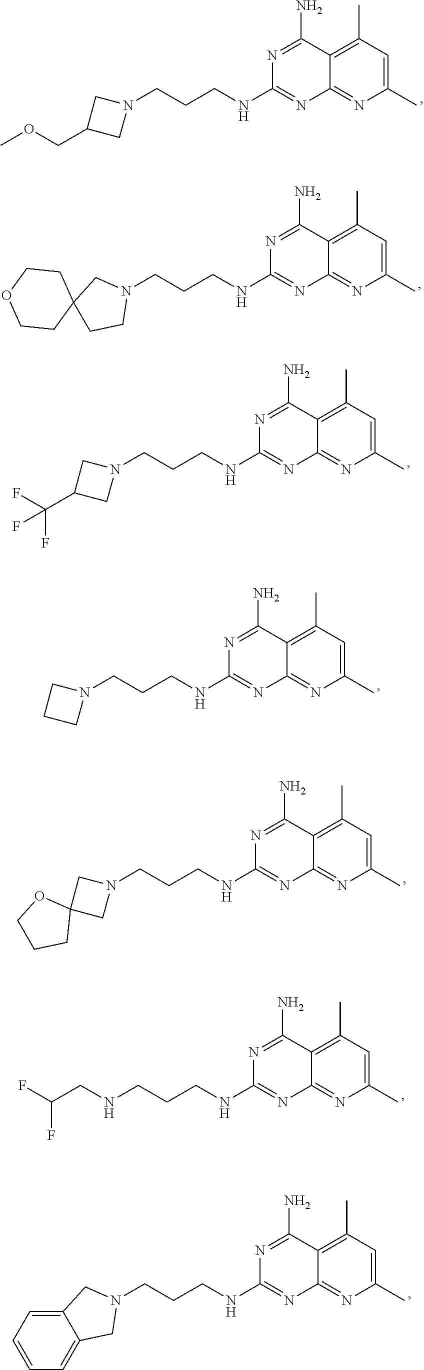

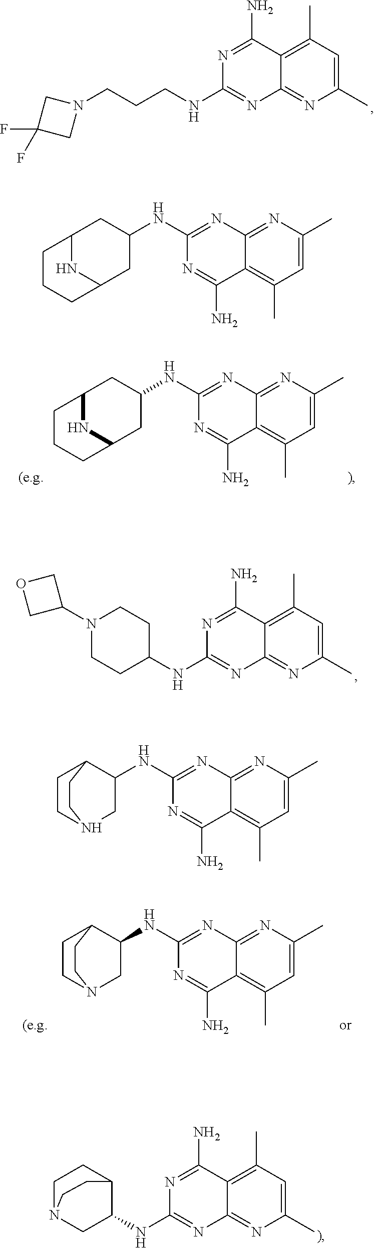

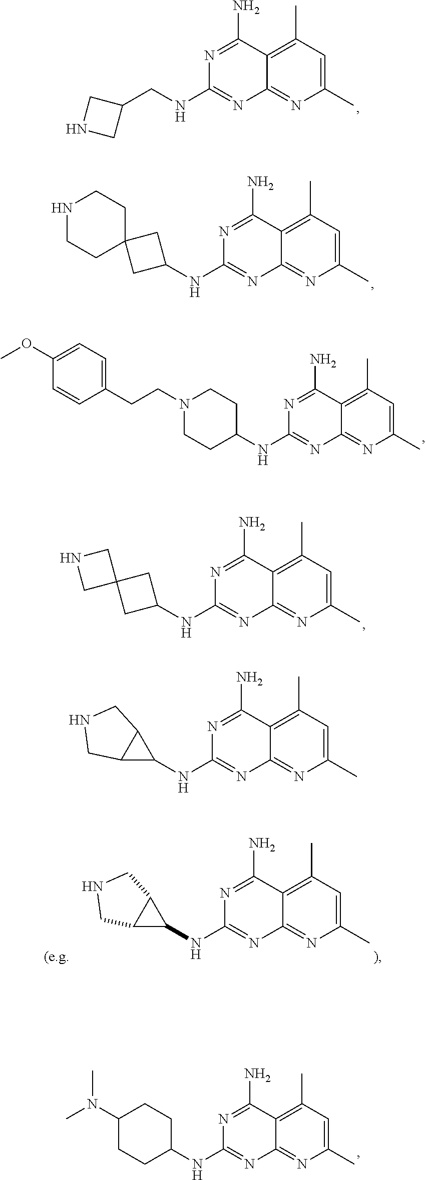

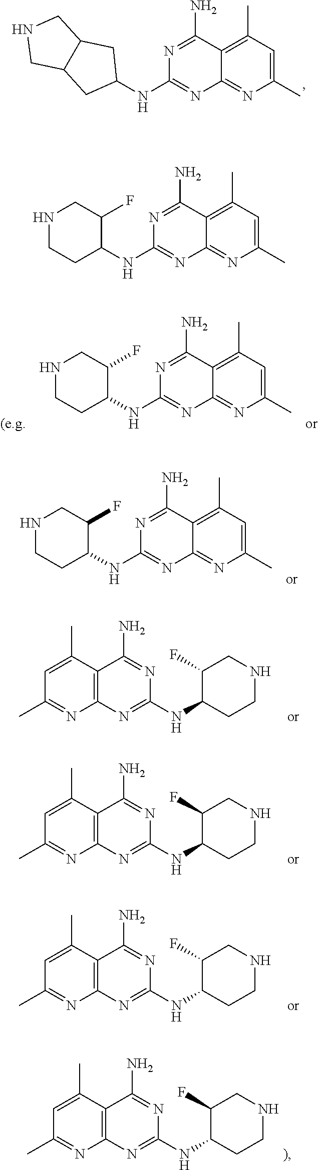

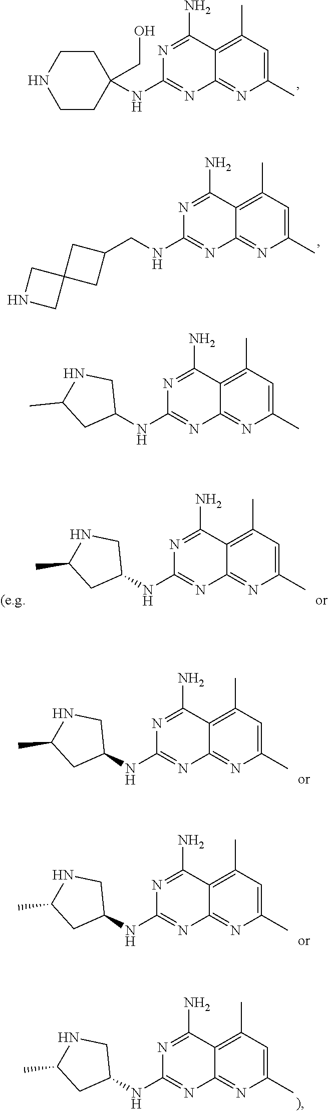

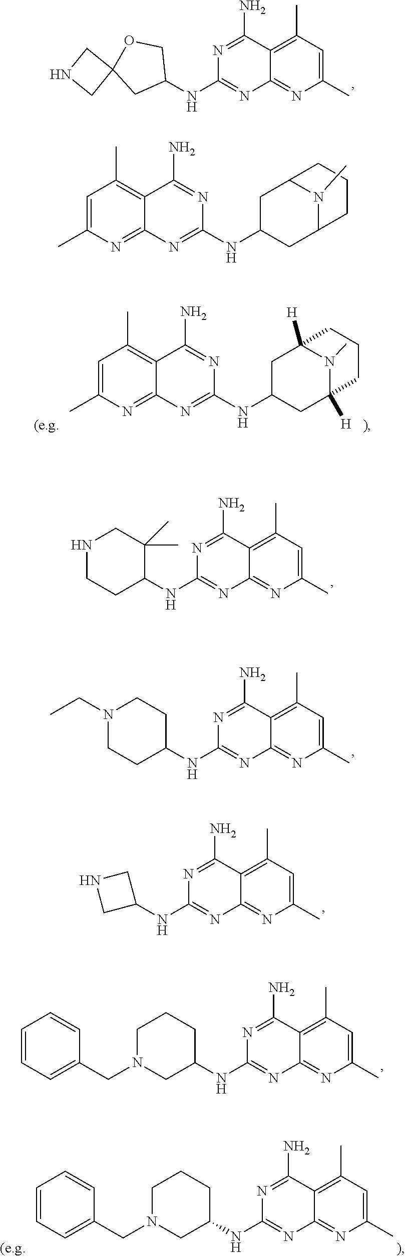

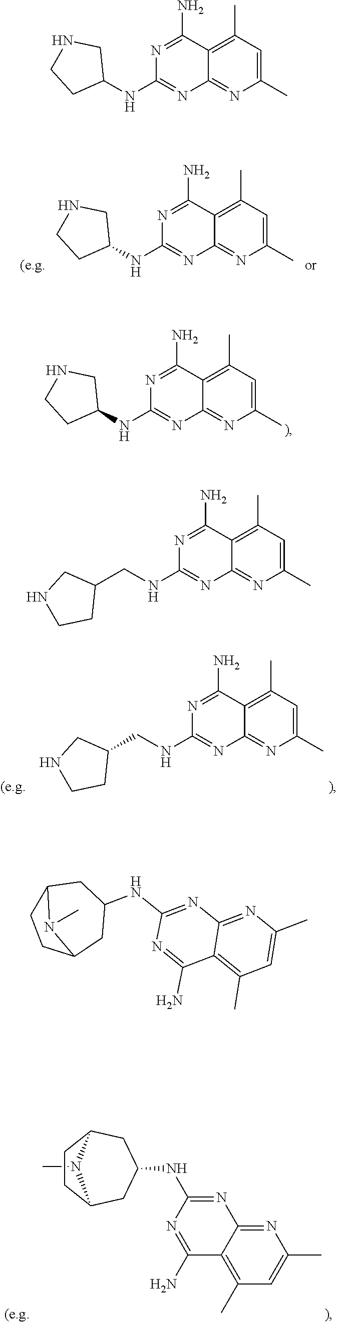

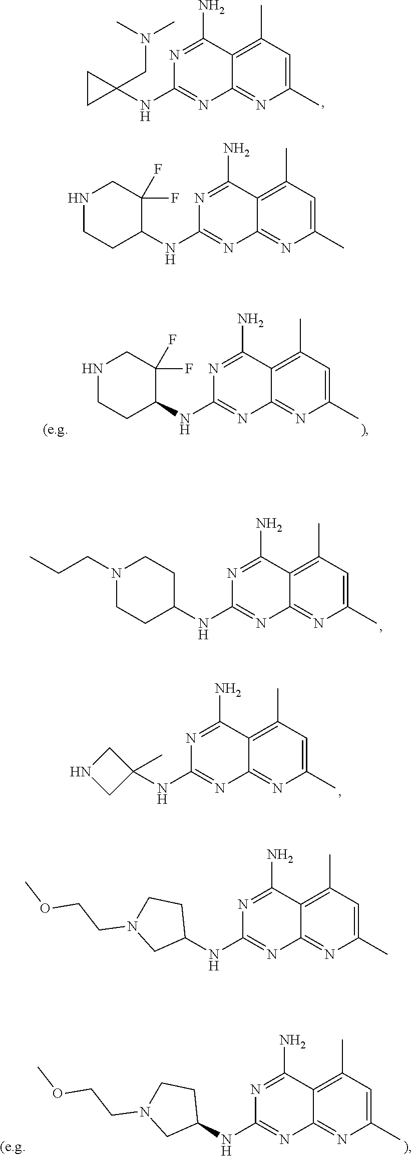

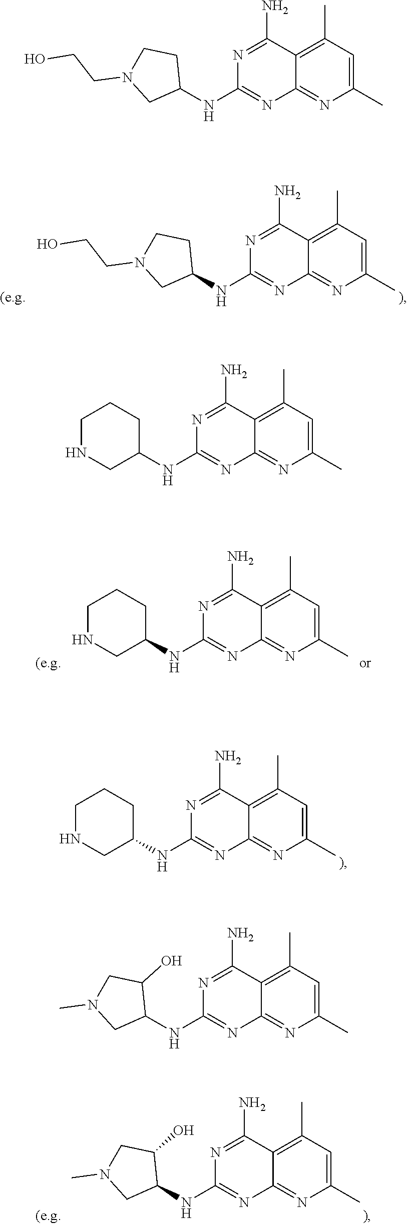

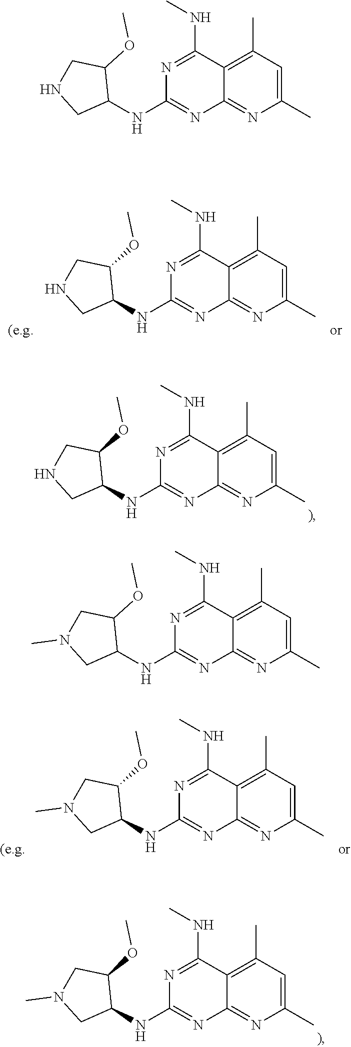

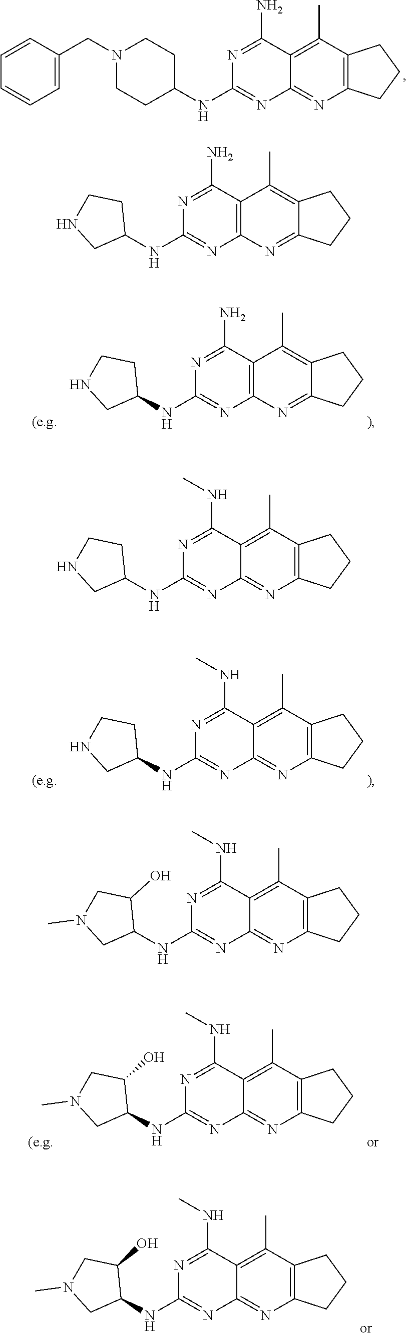

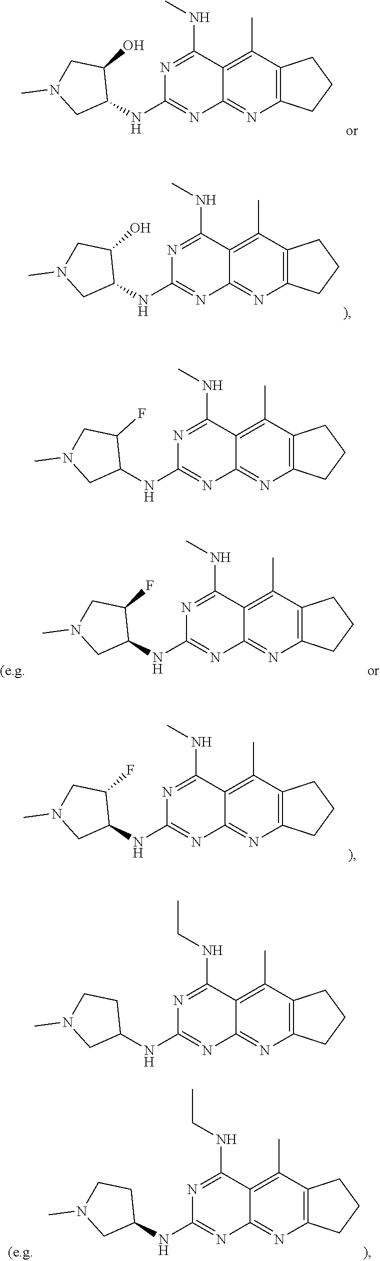

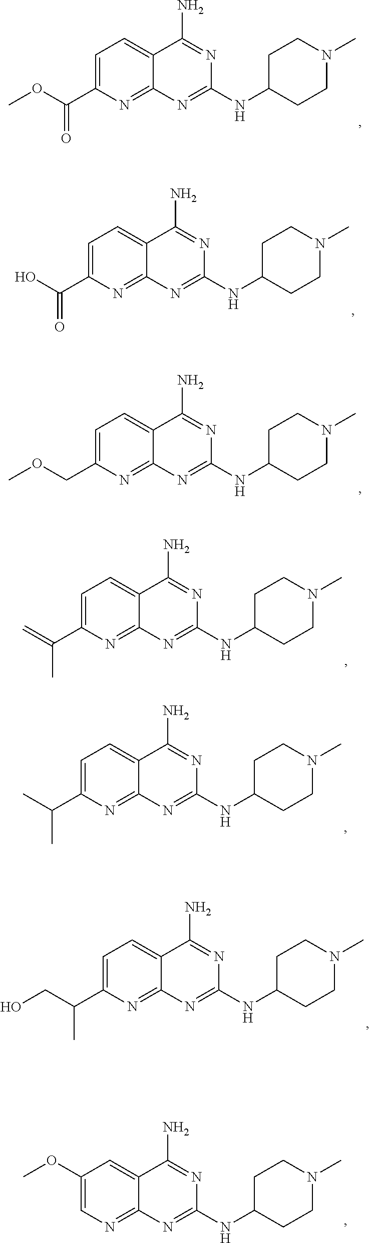

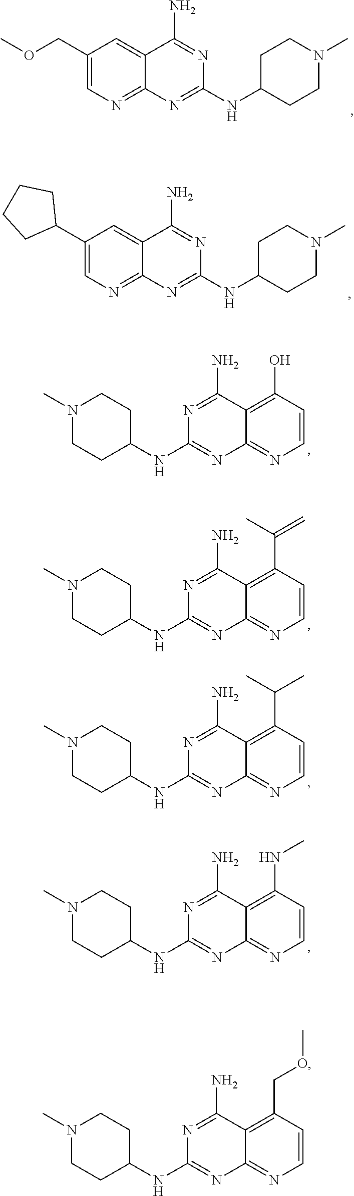

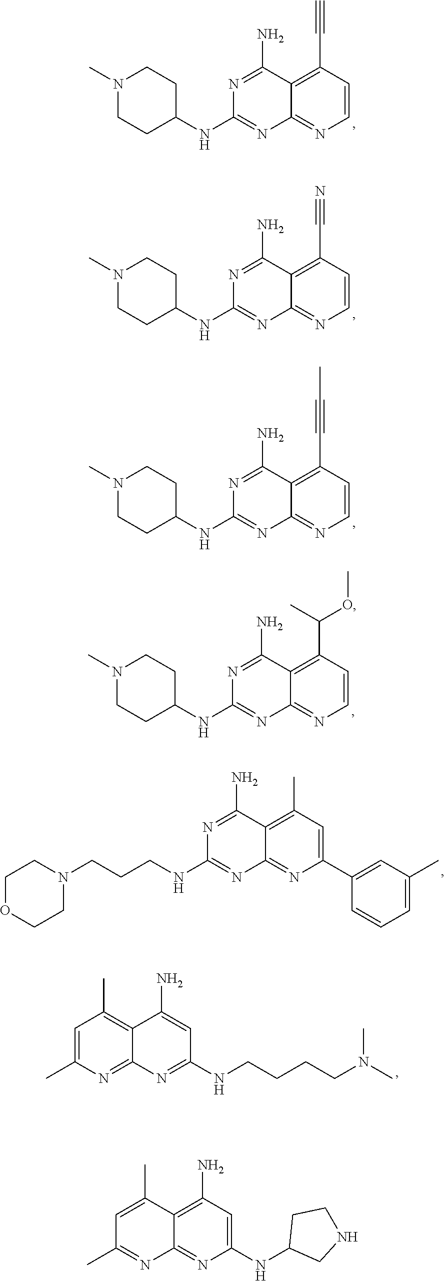

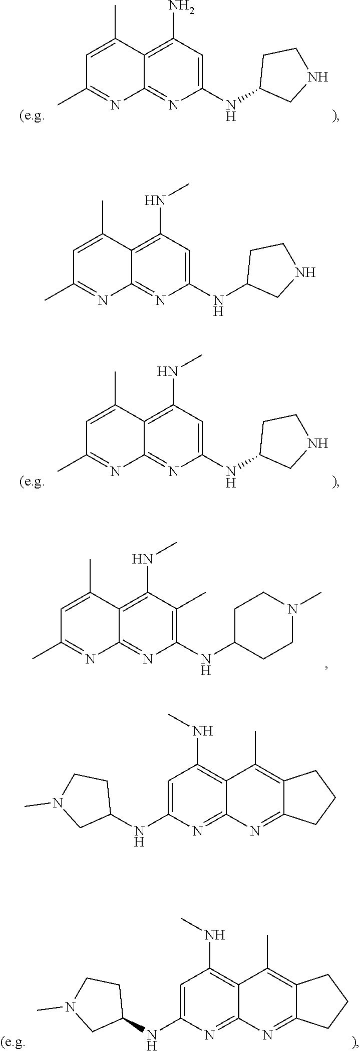

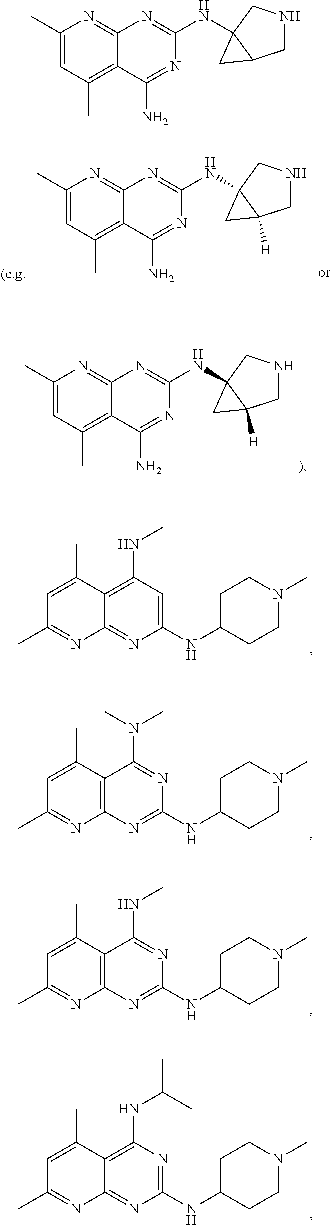

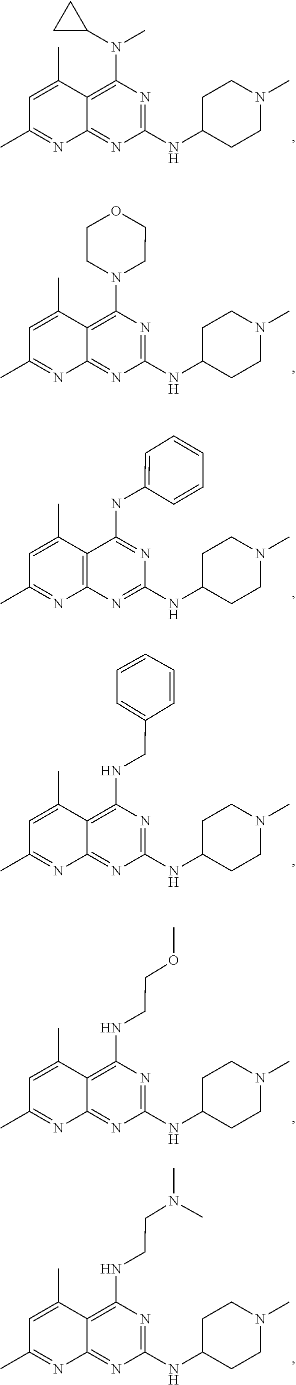

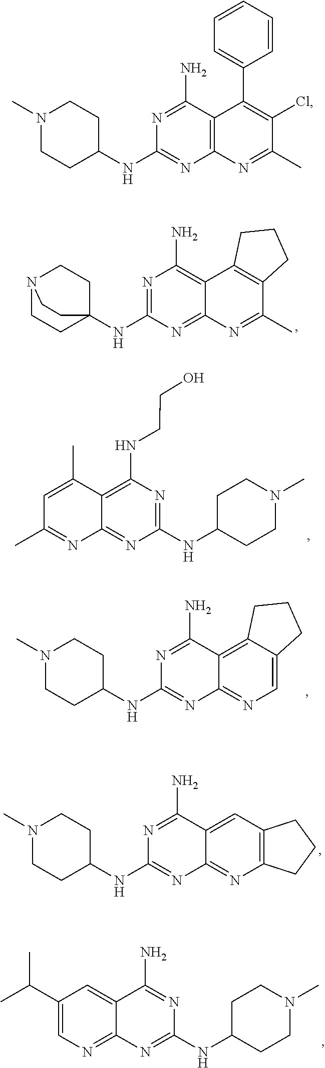

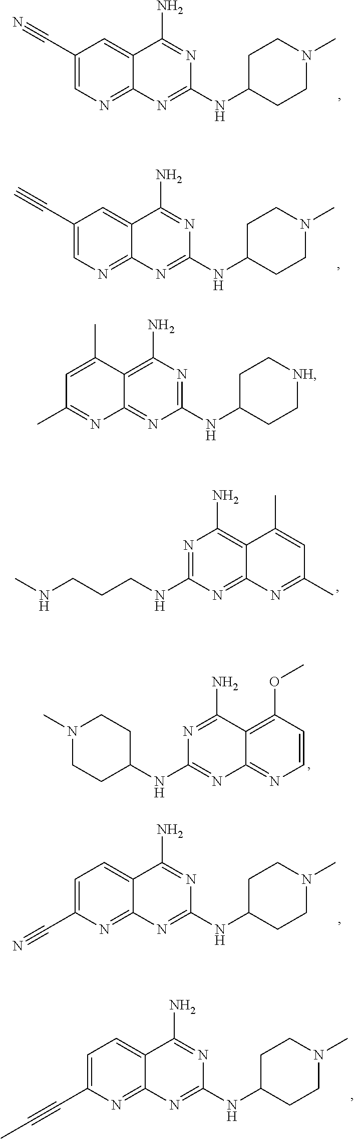

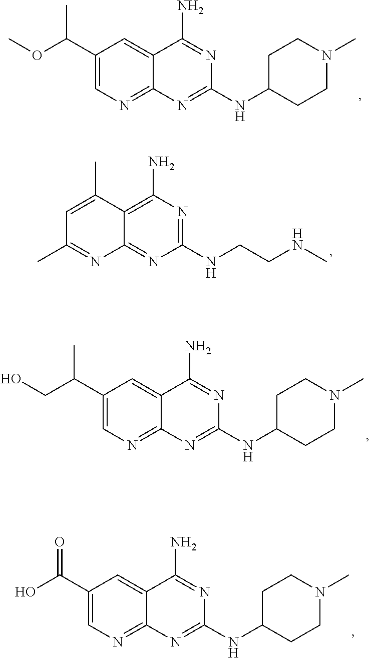

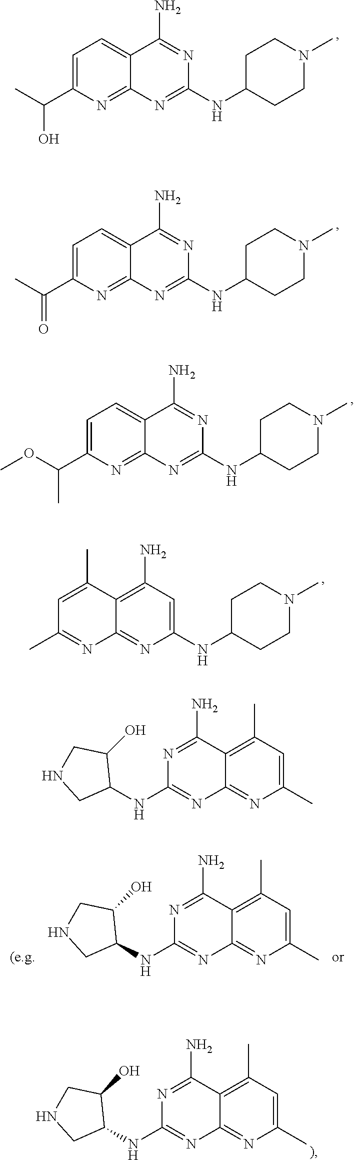

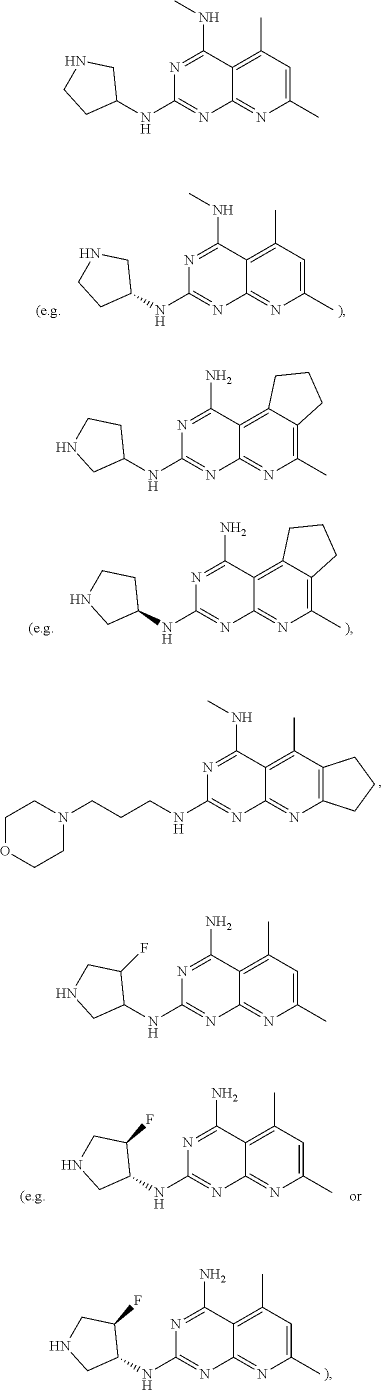

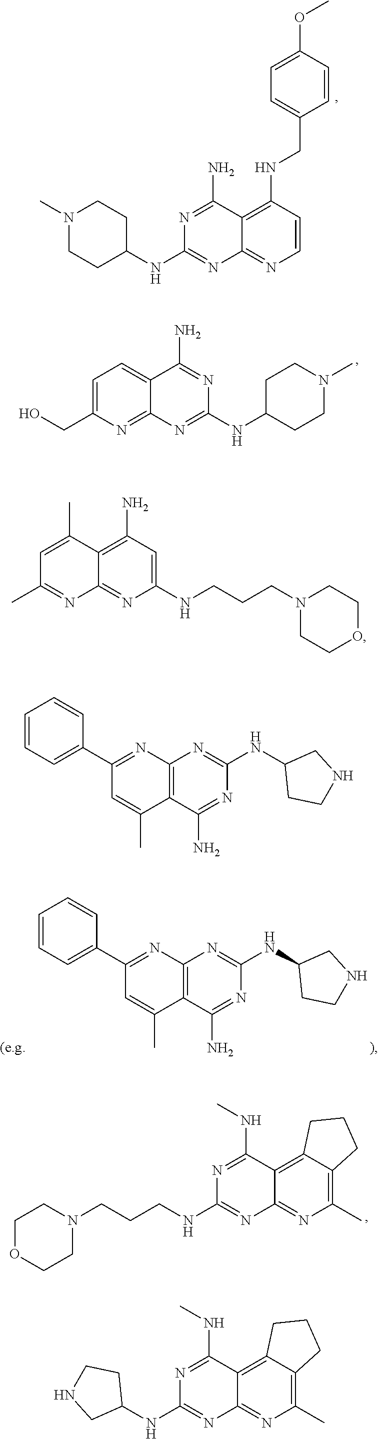

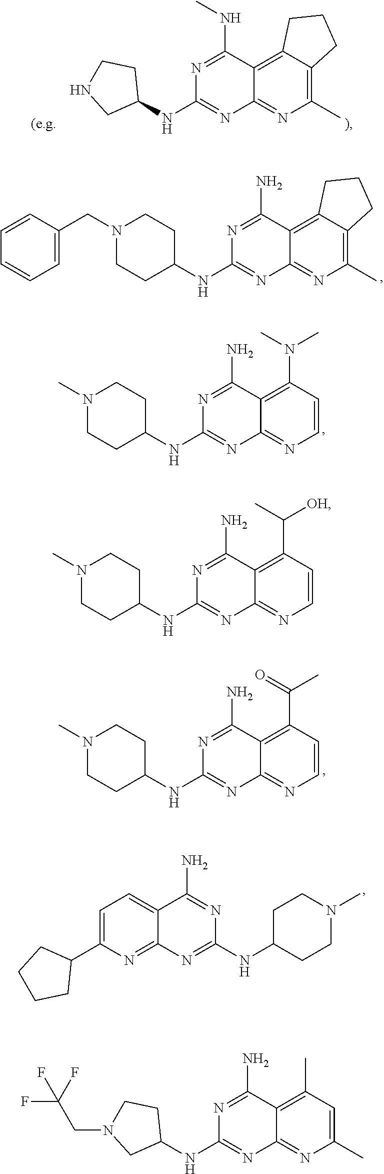

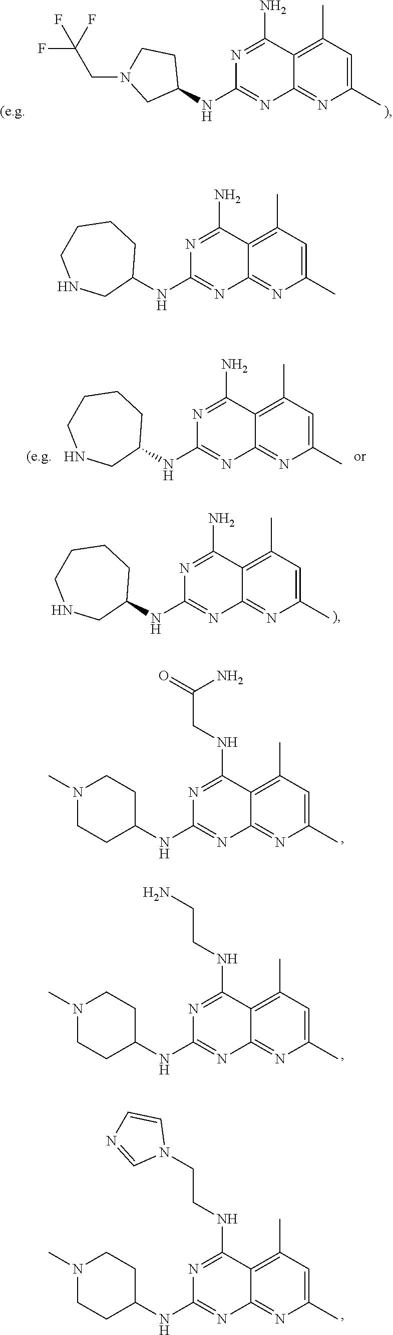

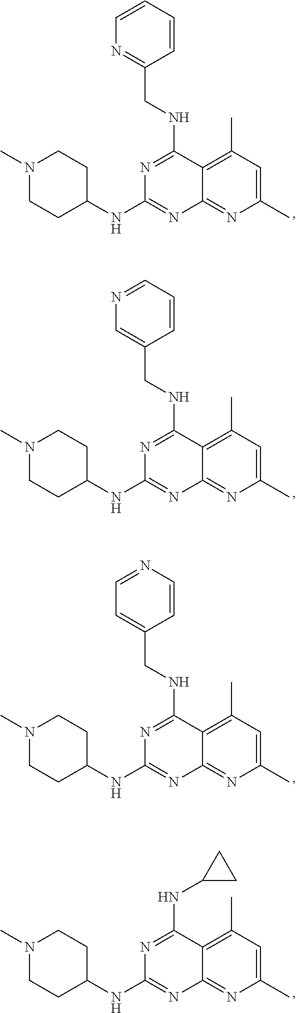

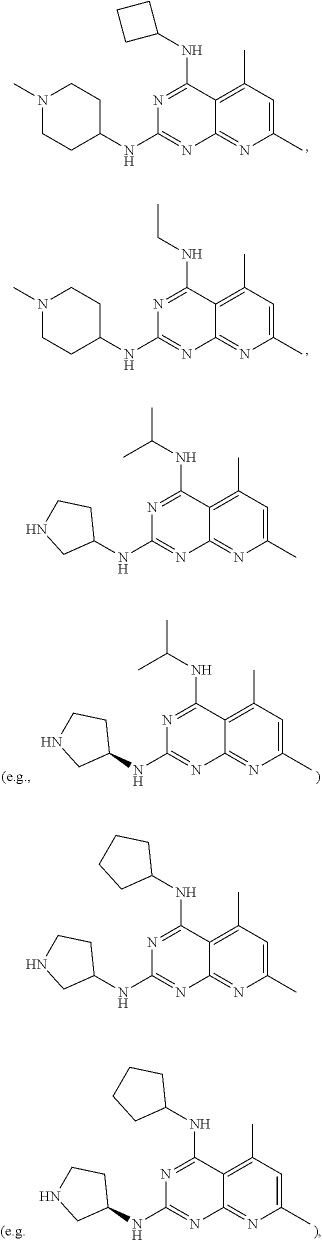

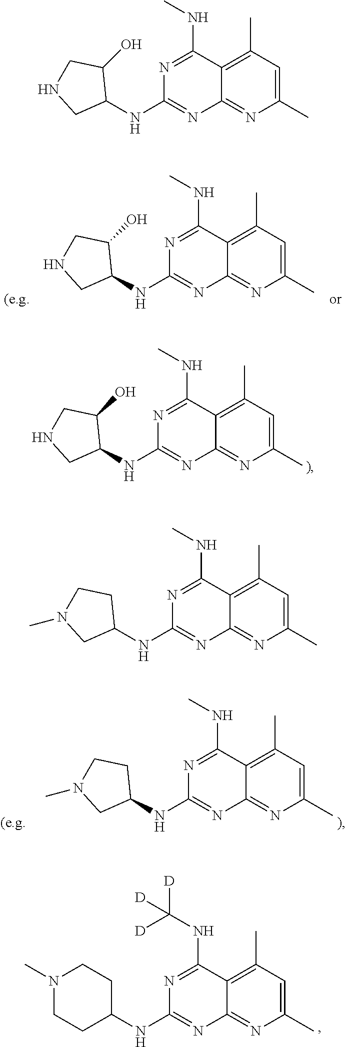

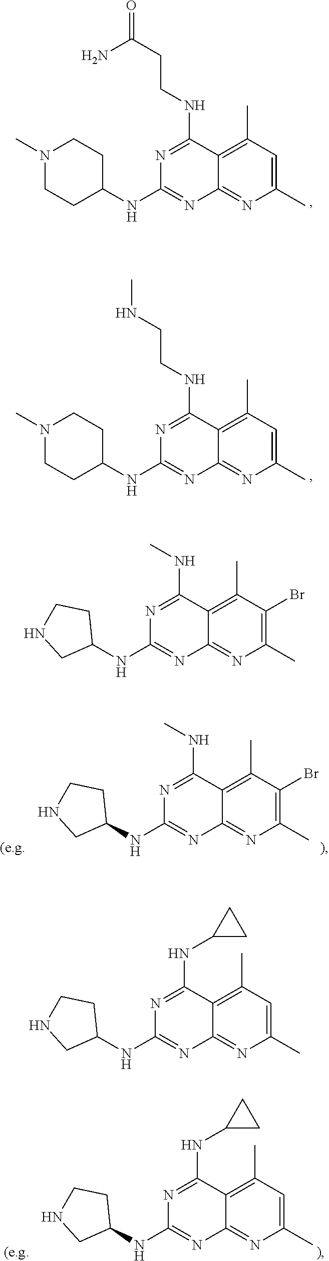

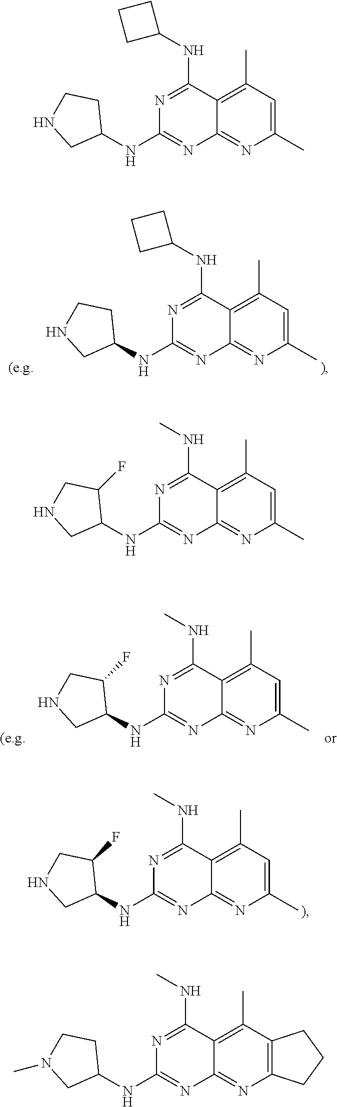

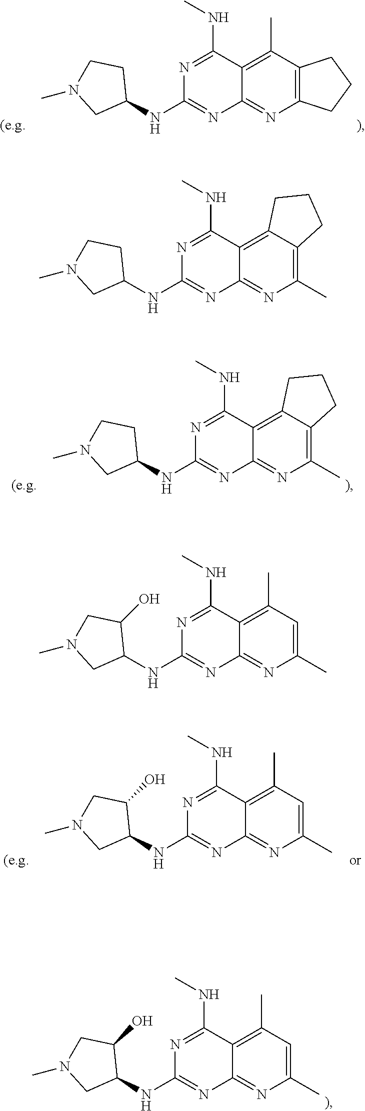

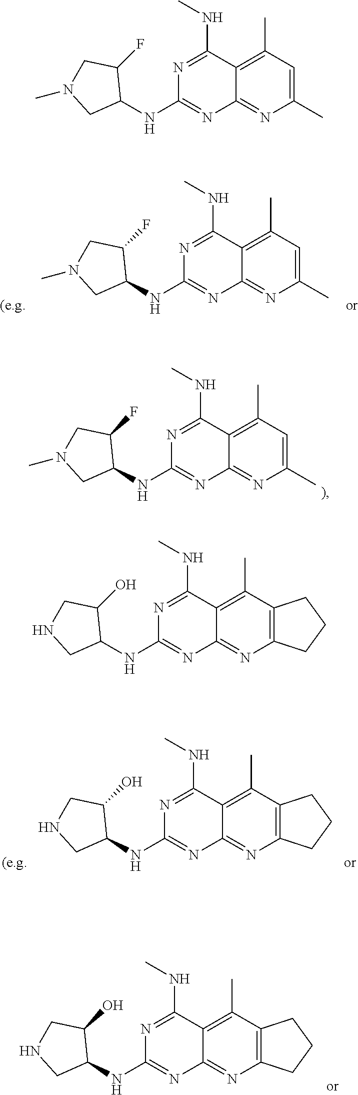

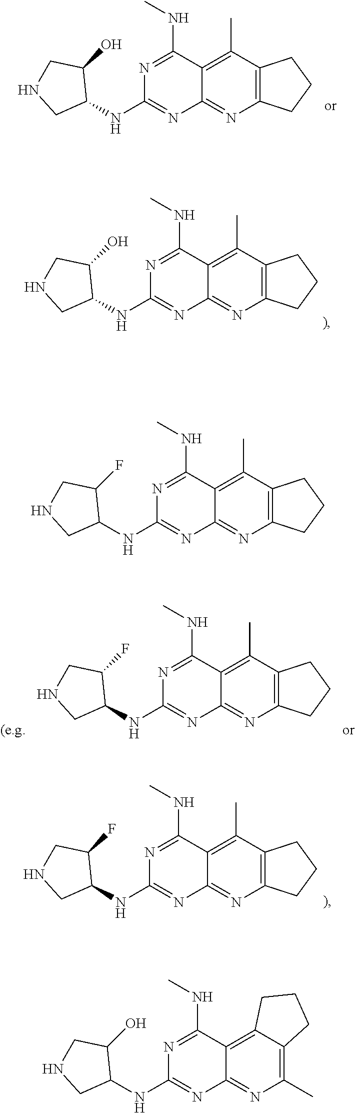 [SMILES: CNc1nc(N[C@@H]2CNC[C@H]2O)nc2nc3c(c(C)c12)CCC3 ; CNc1nc(N[C@@H]2CNC[C@@H]2O)nc2nc3c(c(C)c12)CCC3 ; CNc1nc(NC2CNCC2F)nc2nc3c(c(C)c12)CCC3 ; CNc1nc(N[C@H]2CNC[C@@H]2F)nc2nc3c(c(C)c12)CCC3 ; CNc1nc(N[C@H]2CNC[C@H]2F)nc2nc3c(c(C)c12)CCC3 ; CNc1nc(NC2CNCC2O)nc2nc(C)c3c(c12)CCC3]

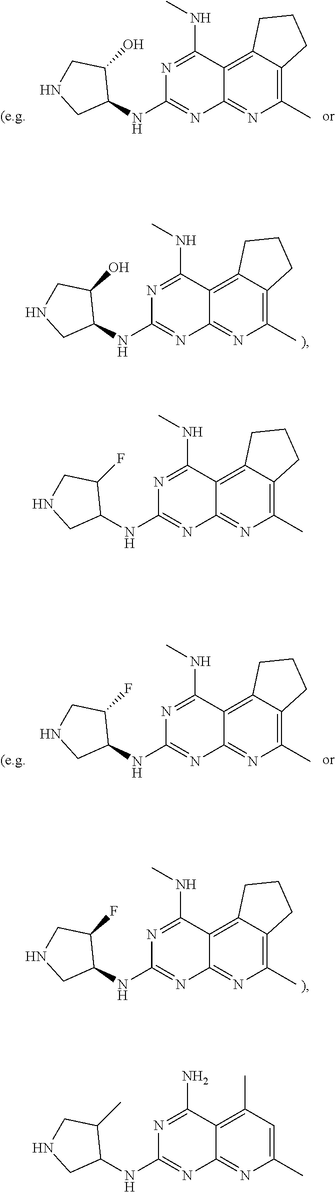 [SMILES: CNc1nc(N[C@H]2CNC[C@@H]2O)nc2nc(C)c3c(c12)CCC3 ; CNc1nc(N[C@H]2CNC[C@H]2O)nc2nc(C)c3c(c12)CCC3 ; CNc1nc(NC2CNCC2F)nc2nc(C)c3c(c12)CCC3 ; CNc1nc(N[C@H]2CNC[C@@H]2F)nc2nc(C)c3c(c12)CCC3 ; CNc1nc(N[C@H]2CNC[C@H]2F)nc2nc(C)c3c(c12)CCC3 ; Cc1cc(C)c2c(N)nc(NC3CNCC3C)nc2n1]

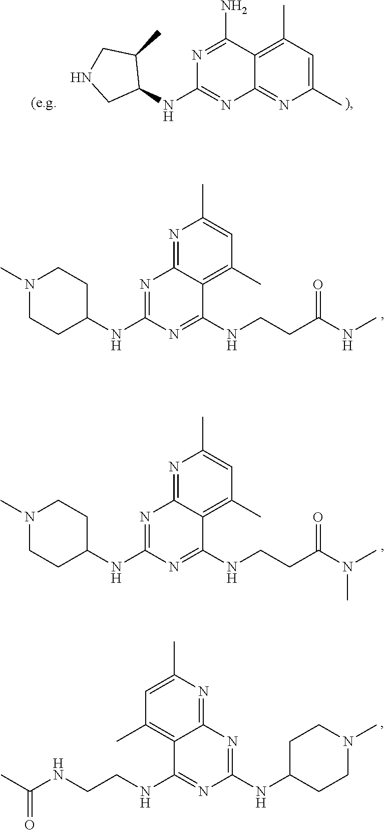

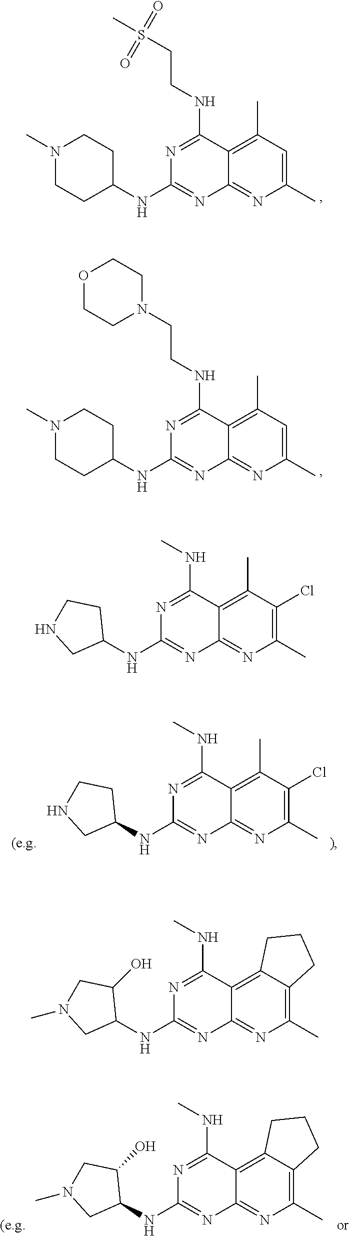

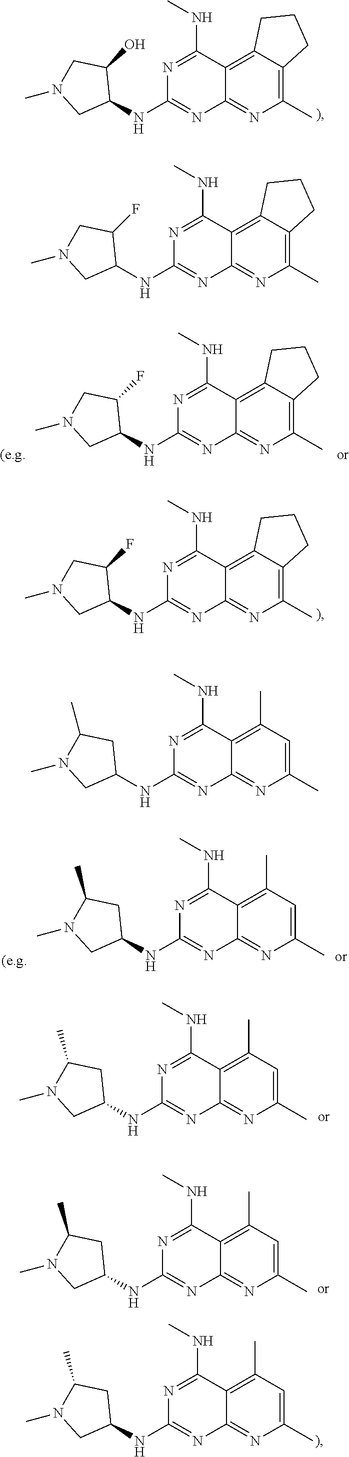

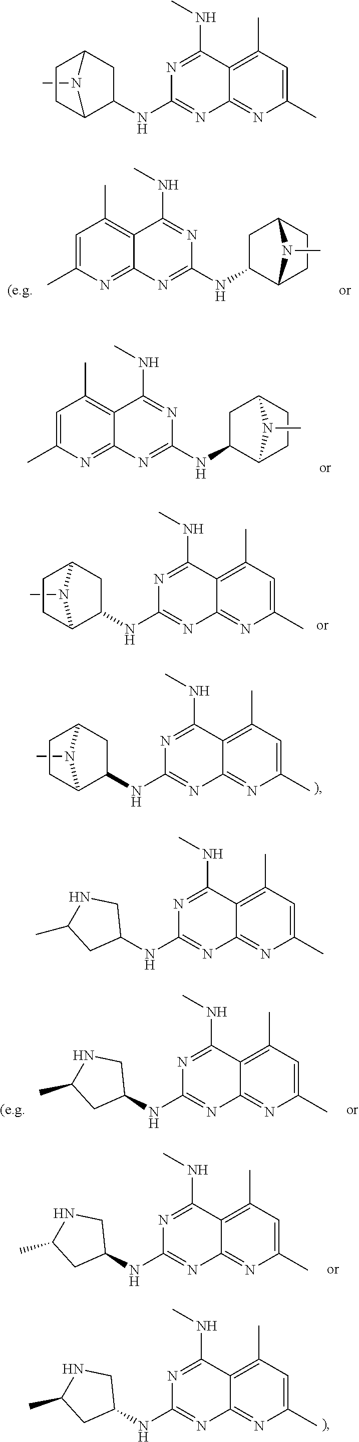

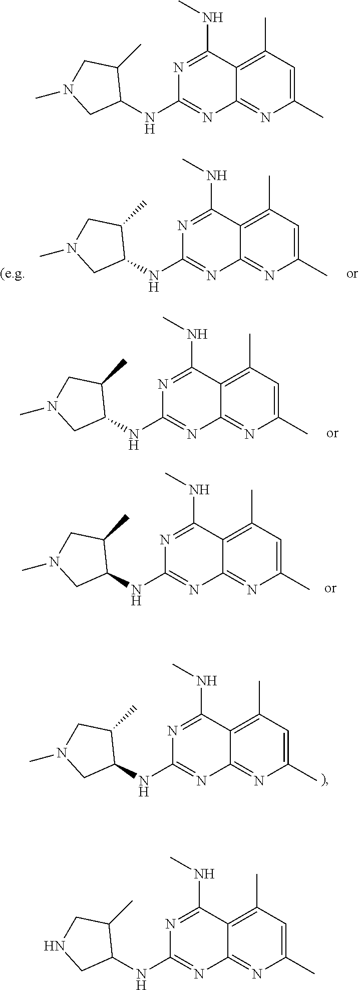

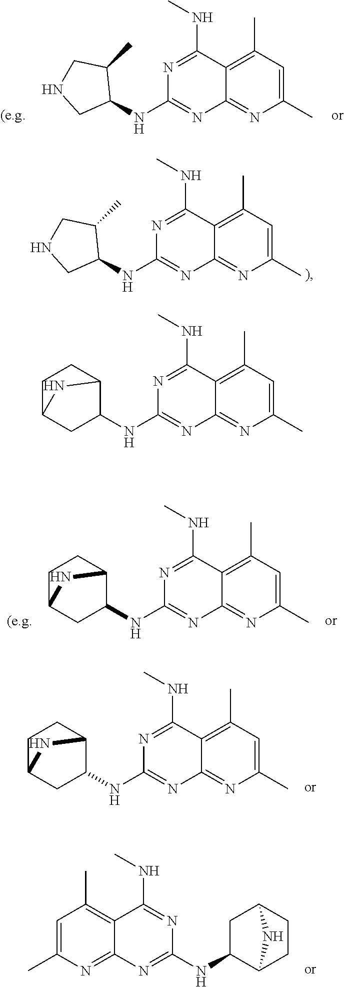

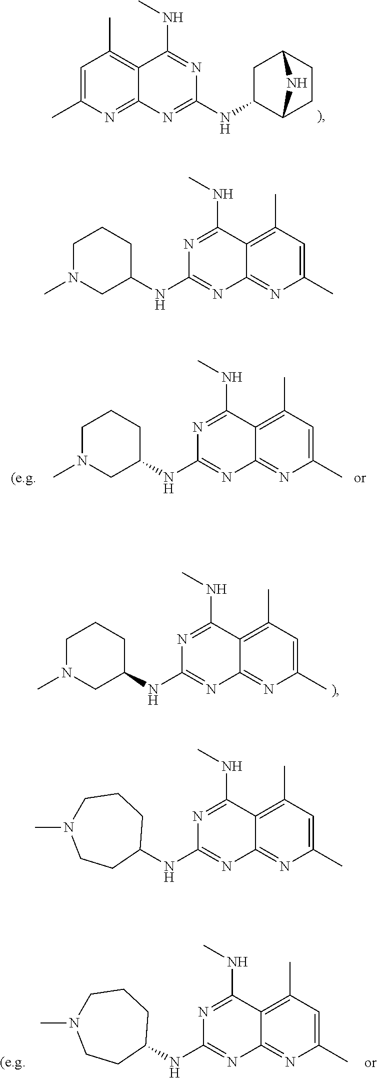

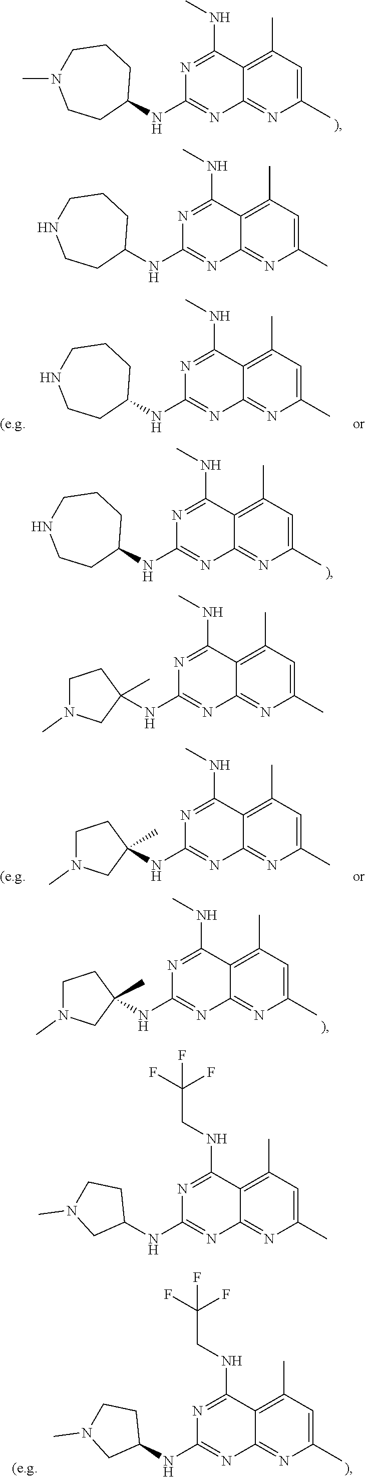

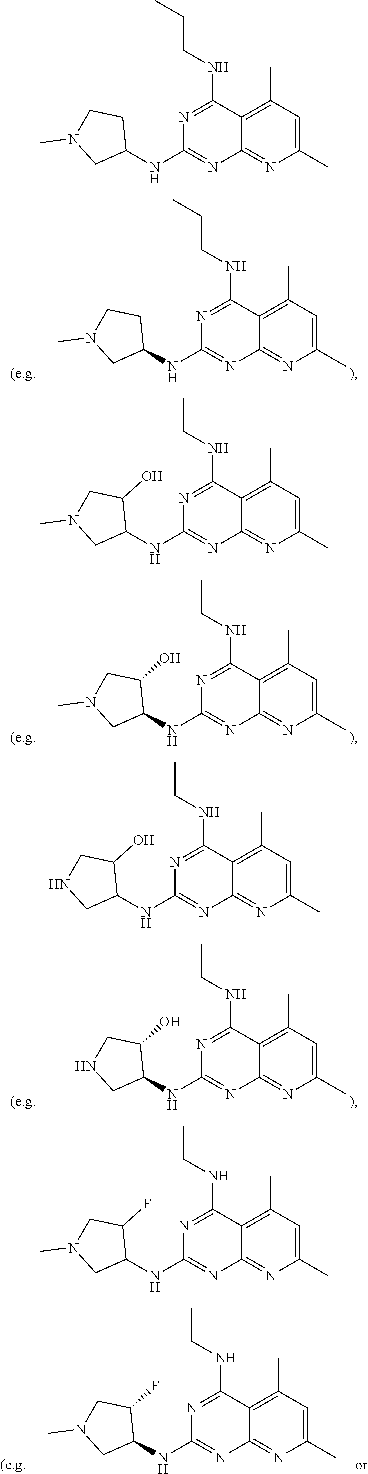

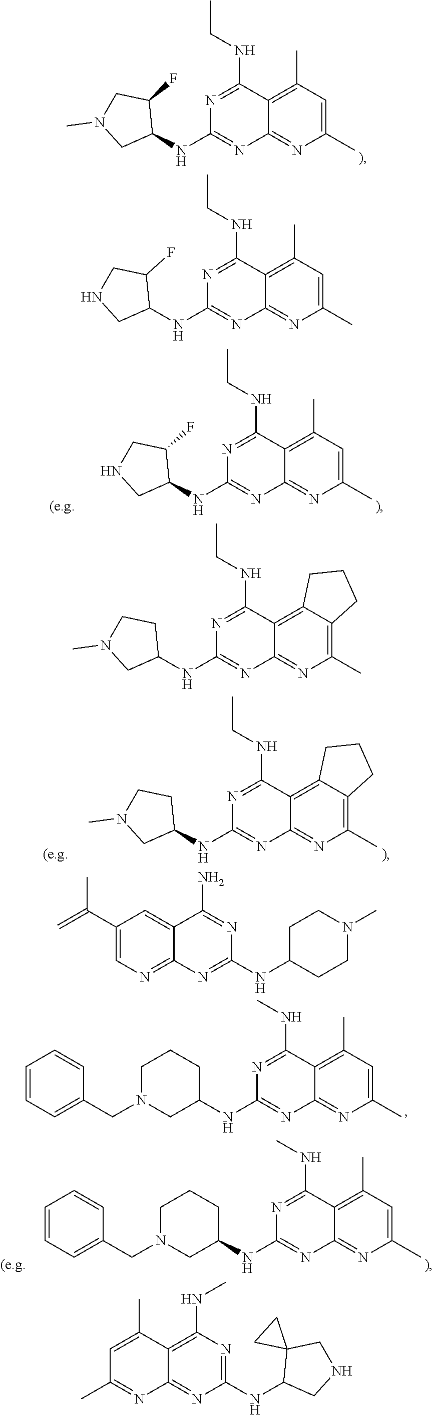

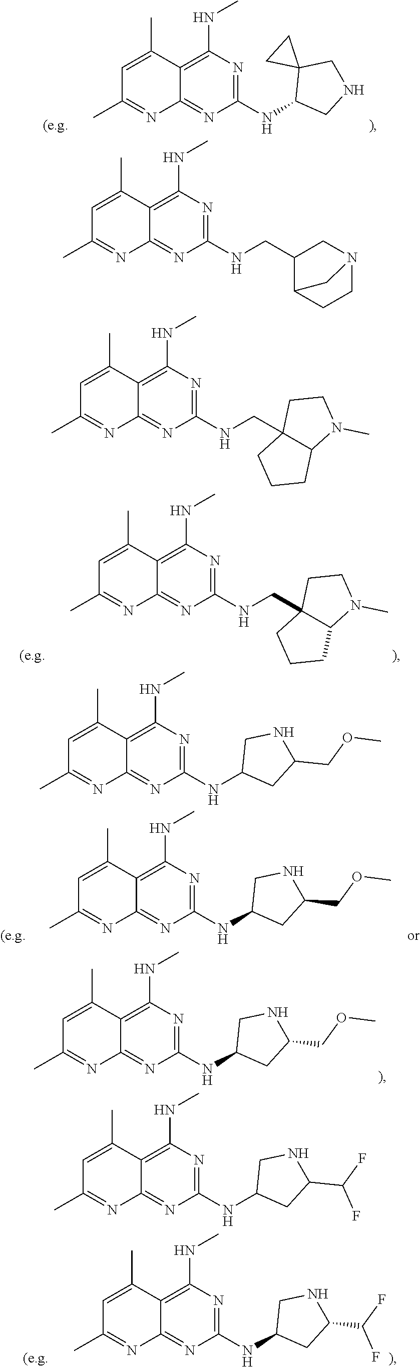

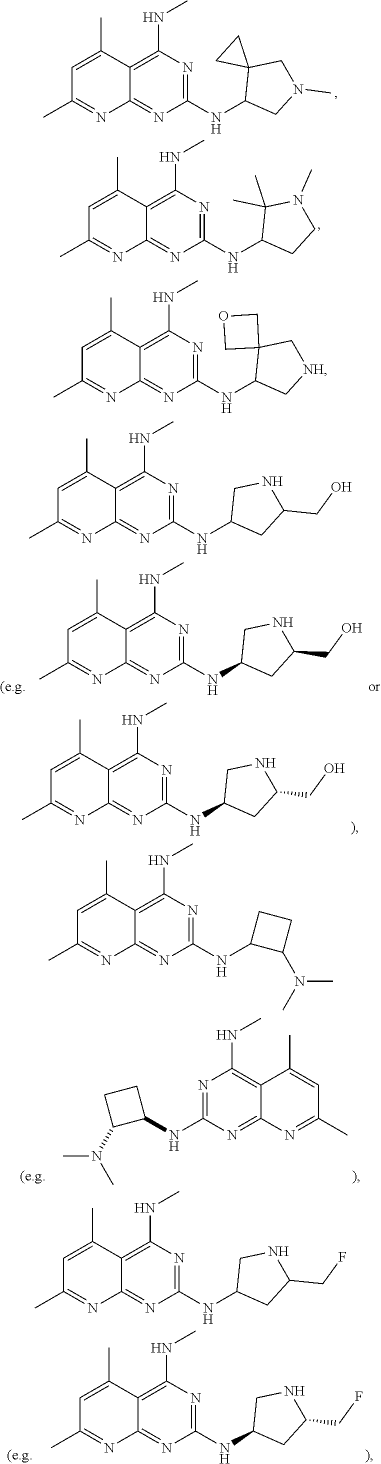

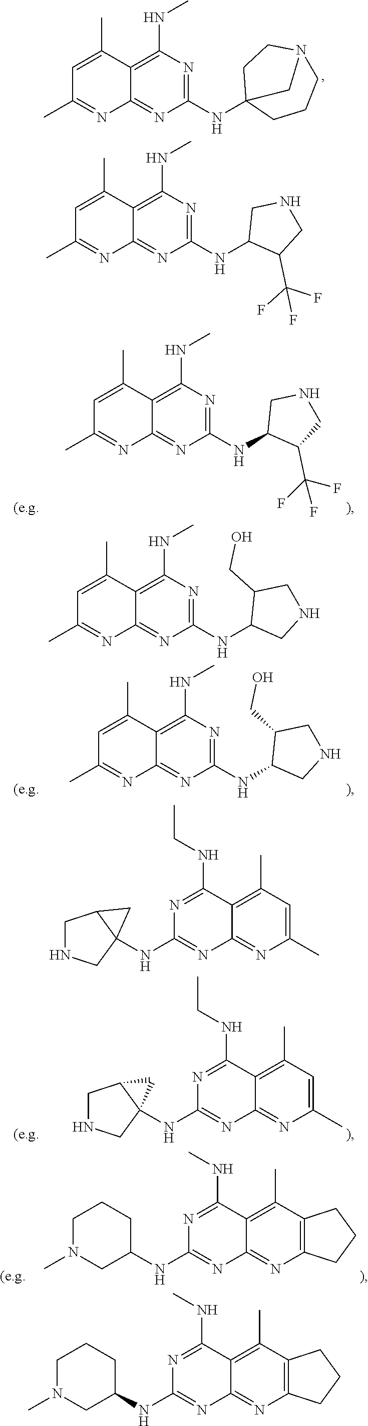

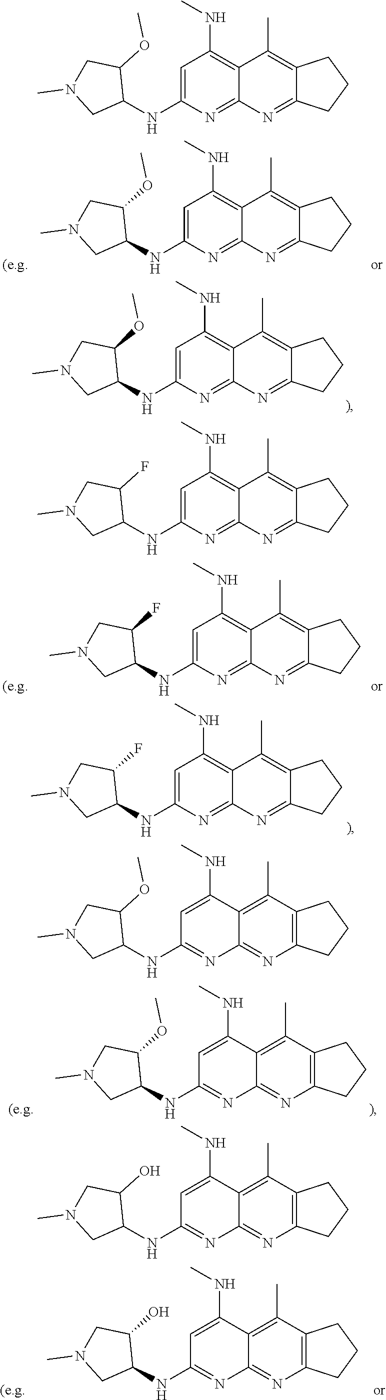

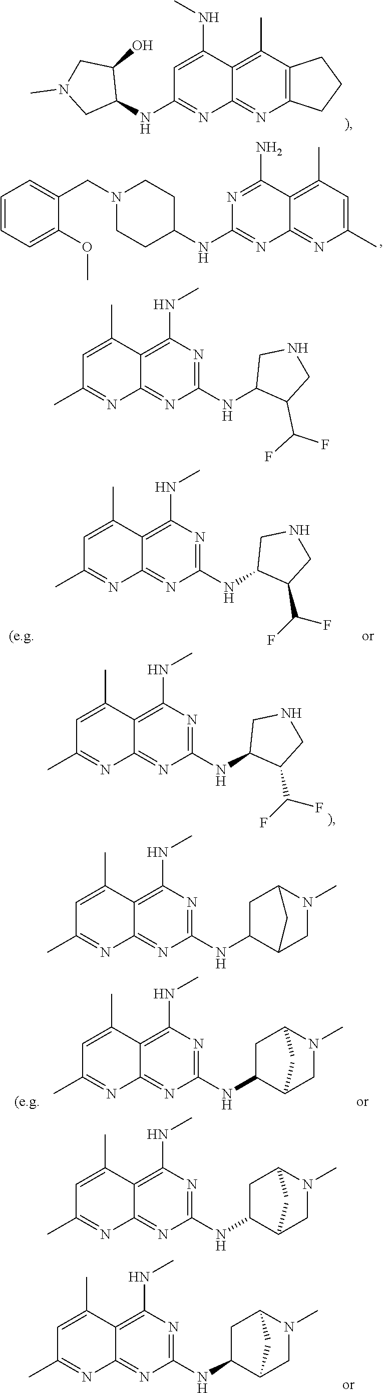

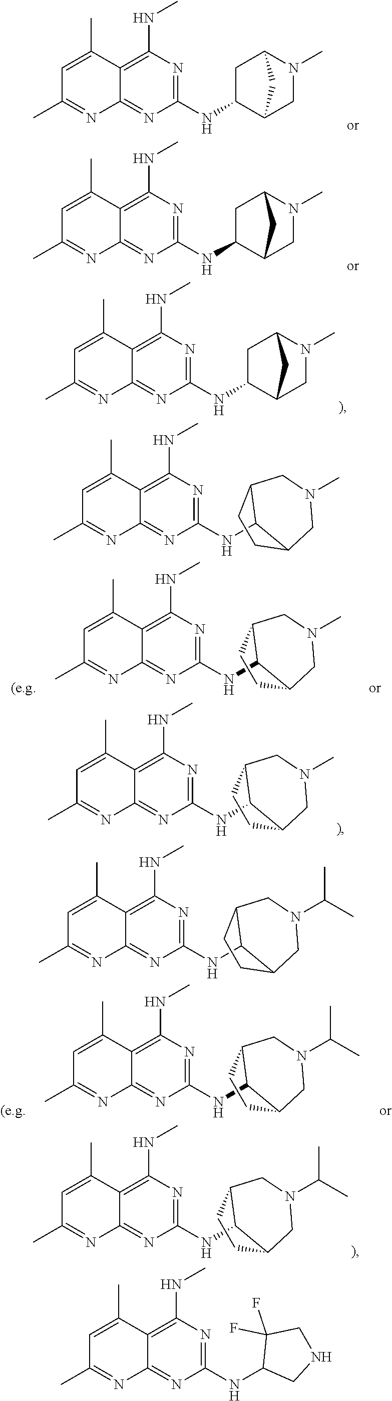

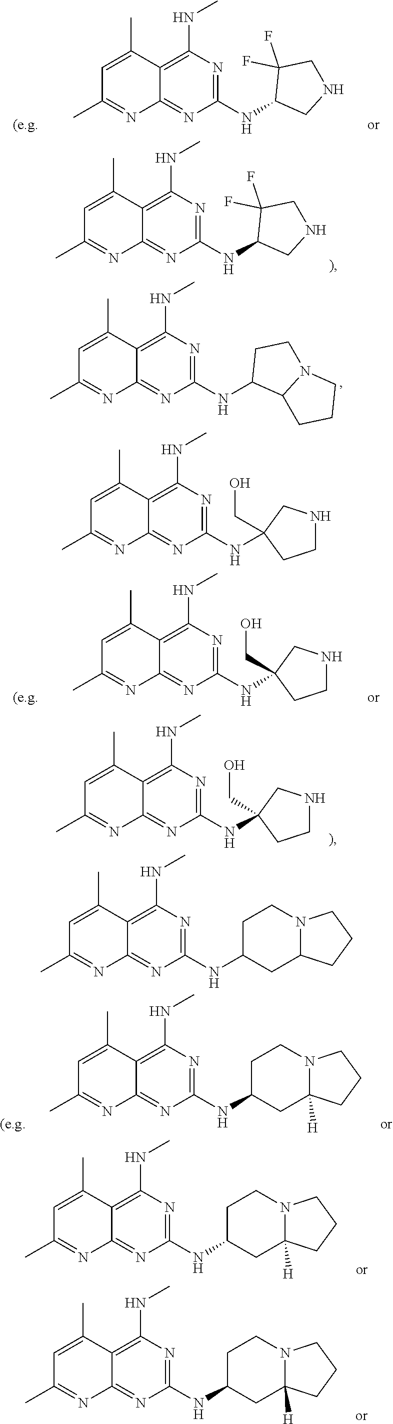

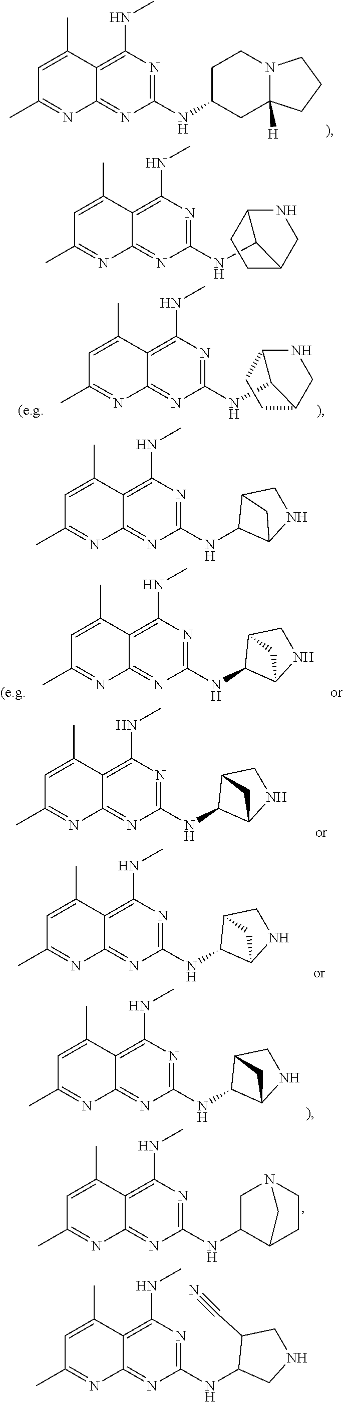

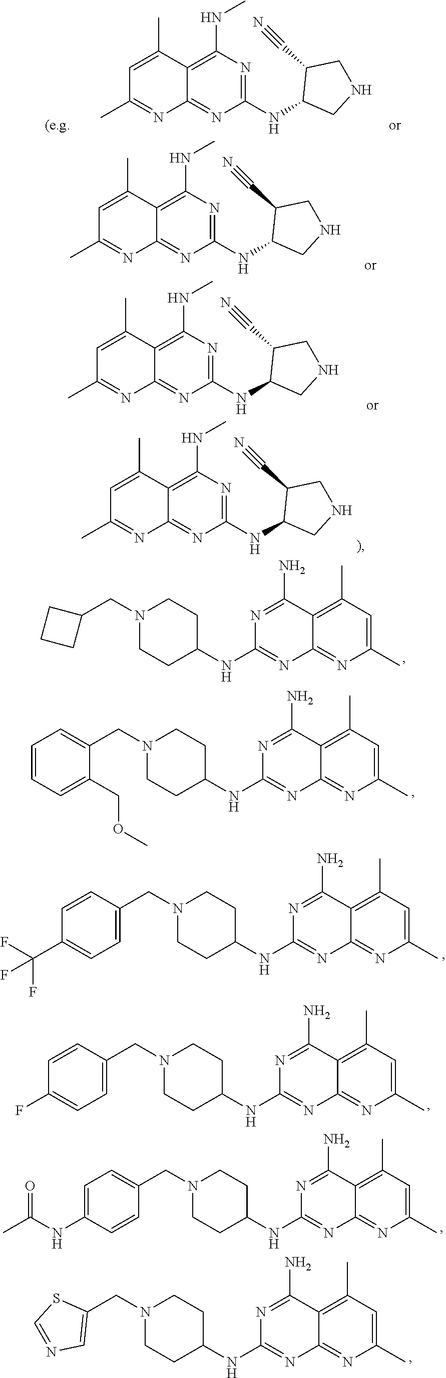

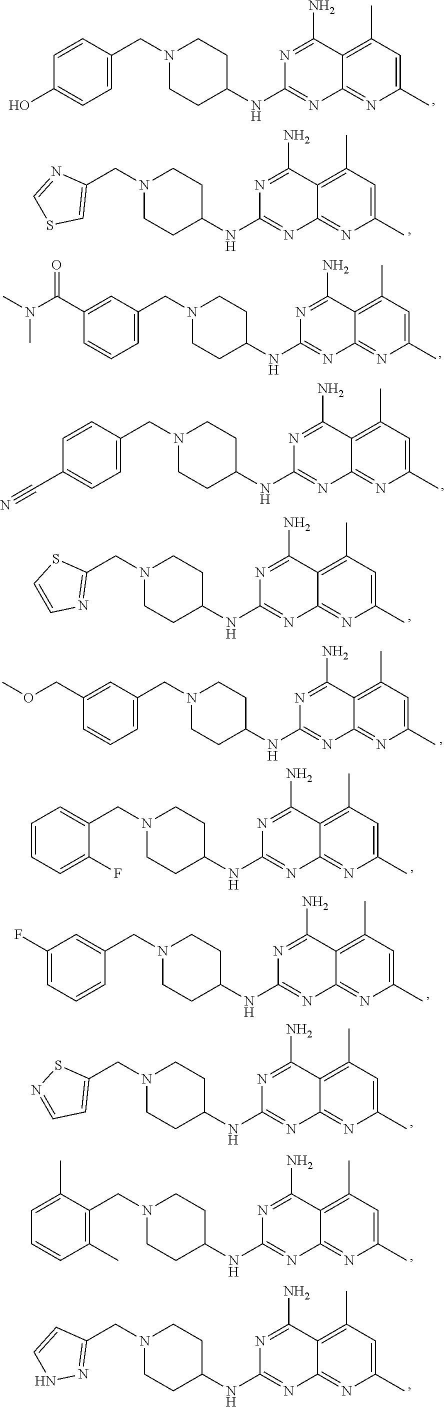

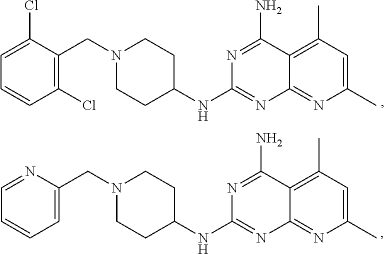

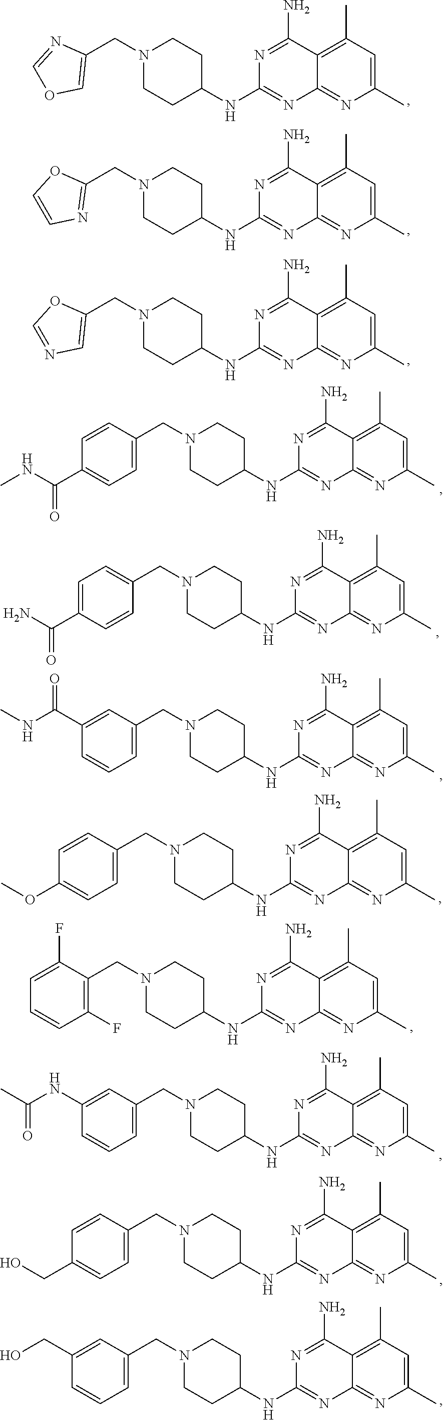

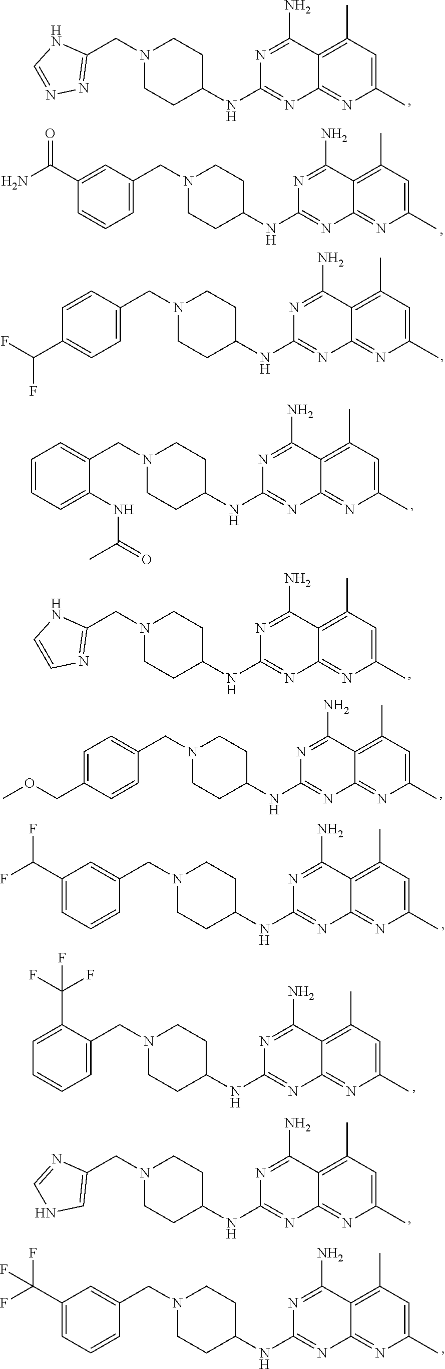

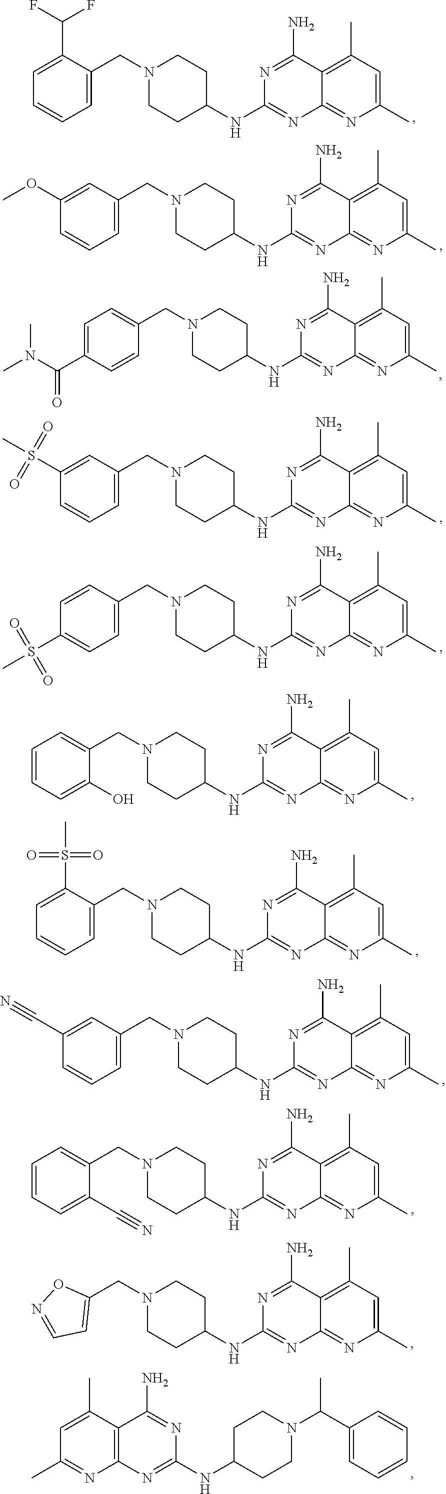

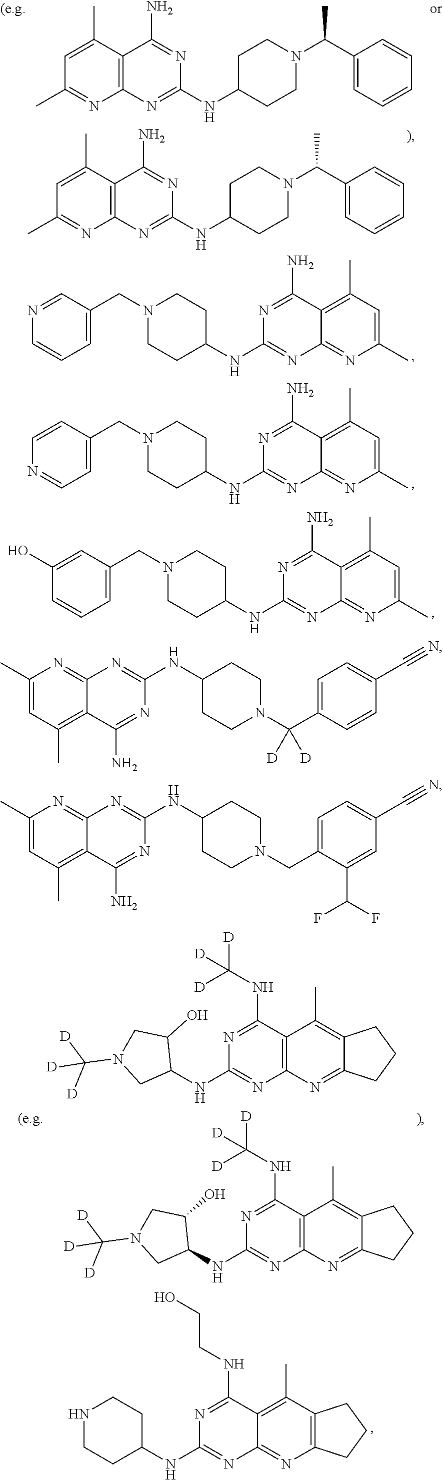

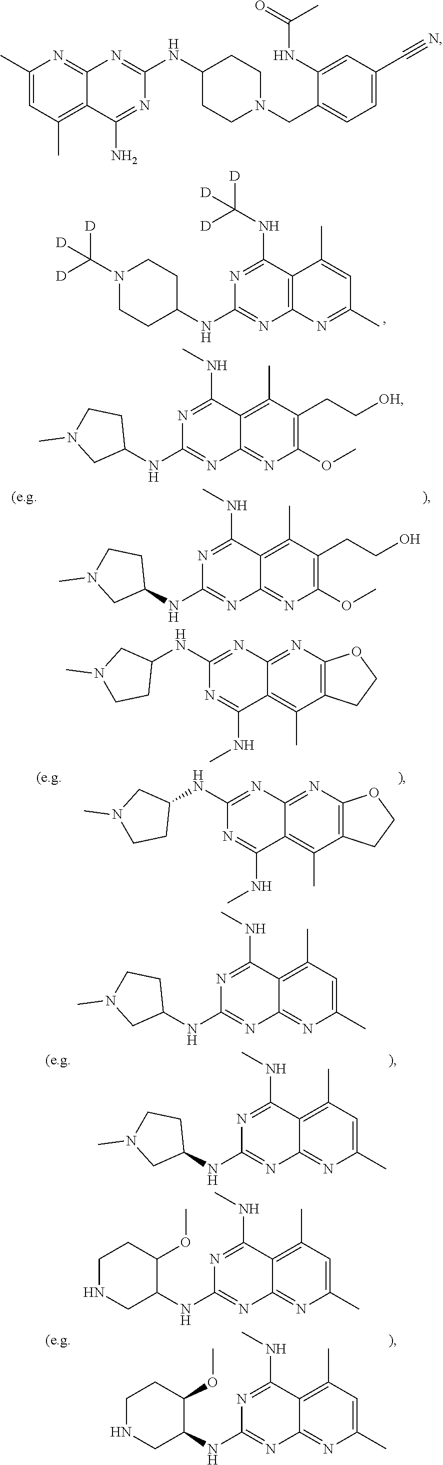

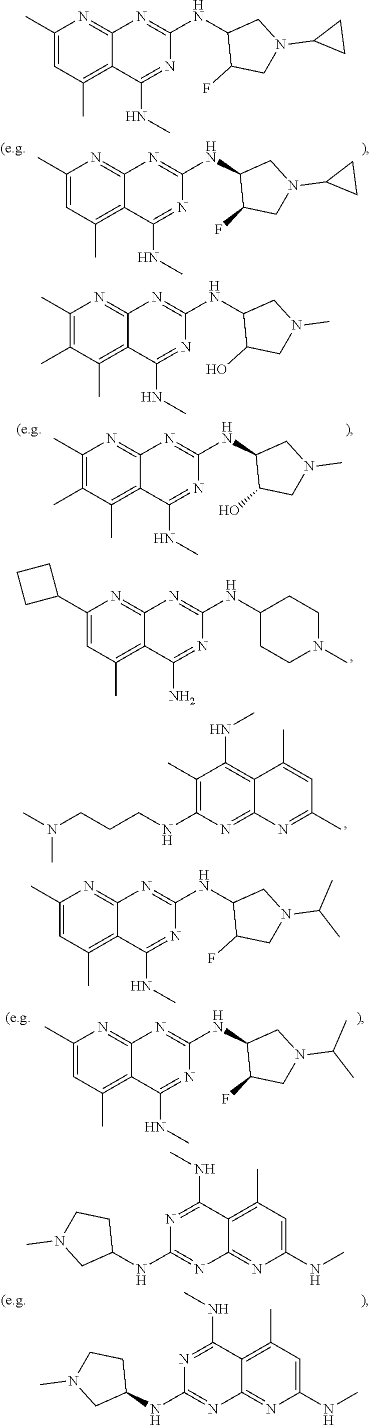

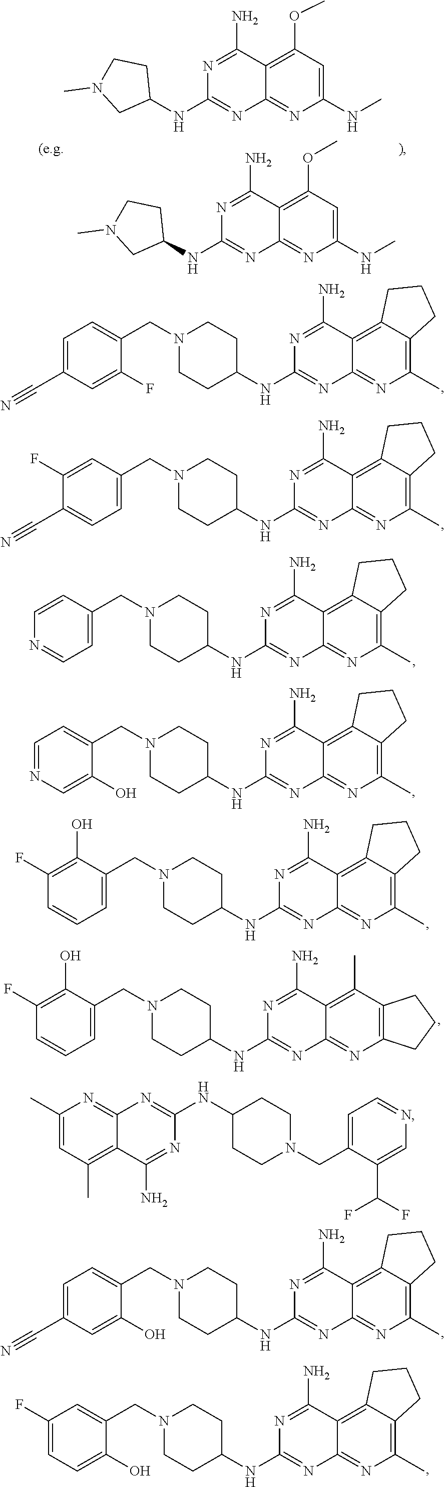

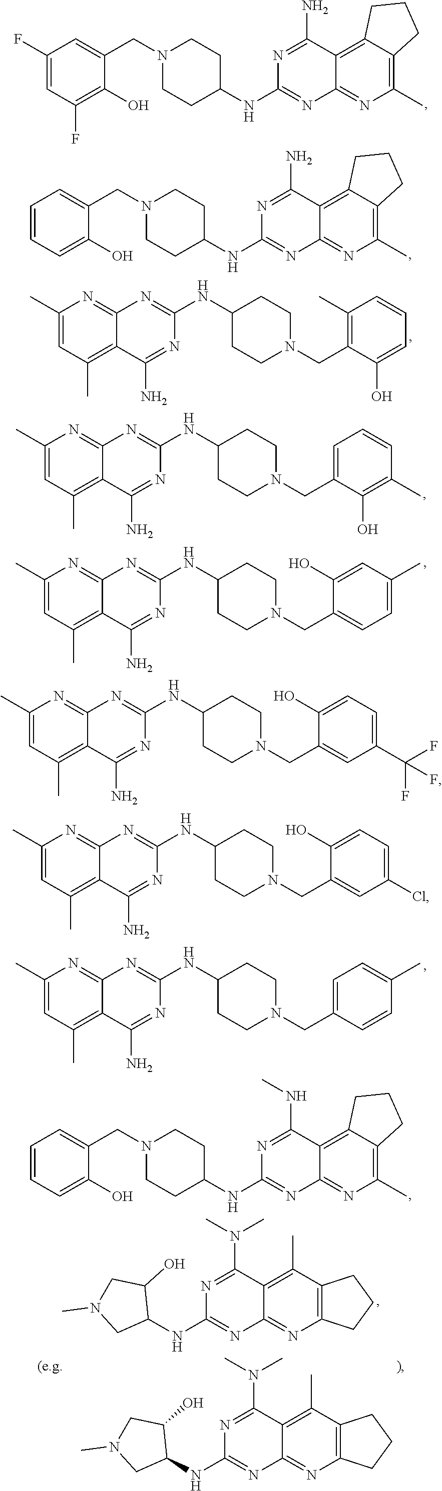

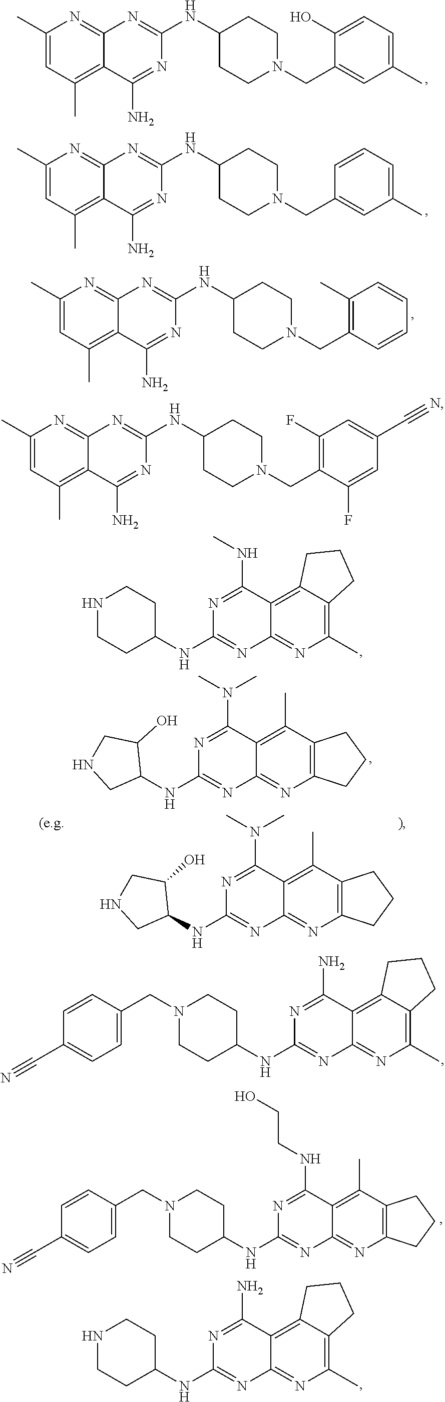

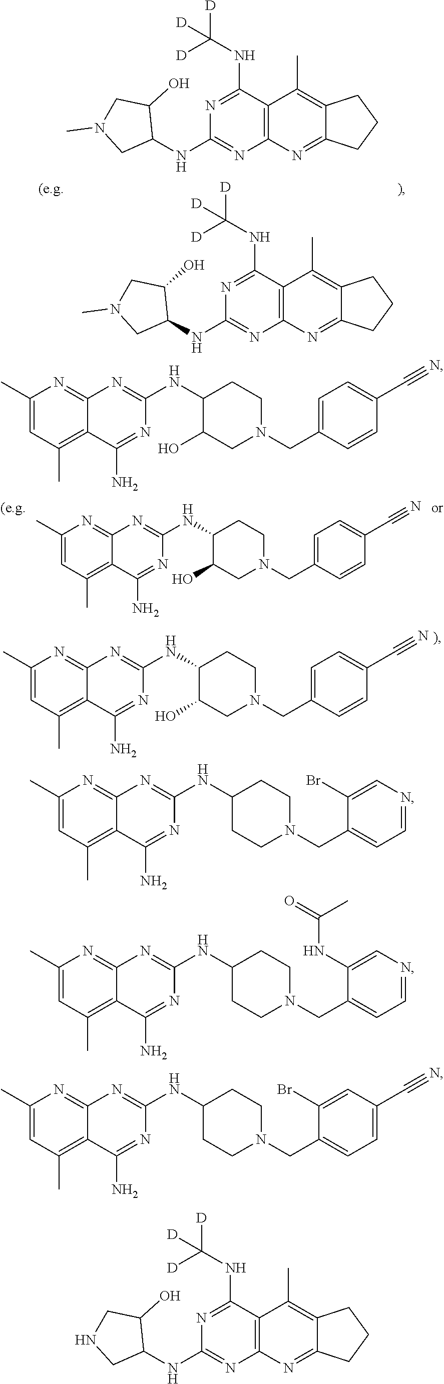

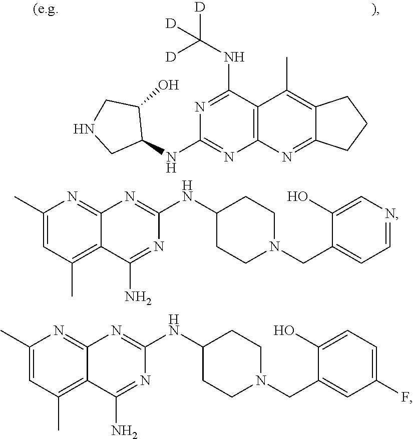

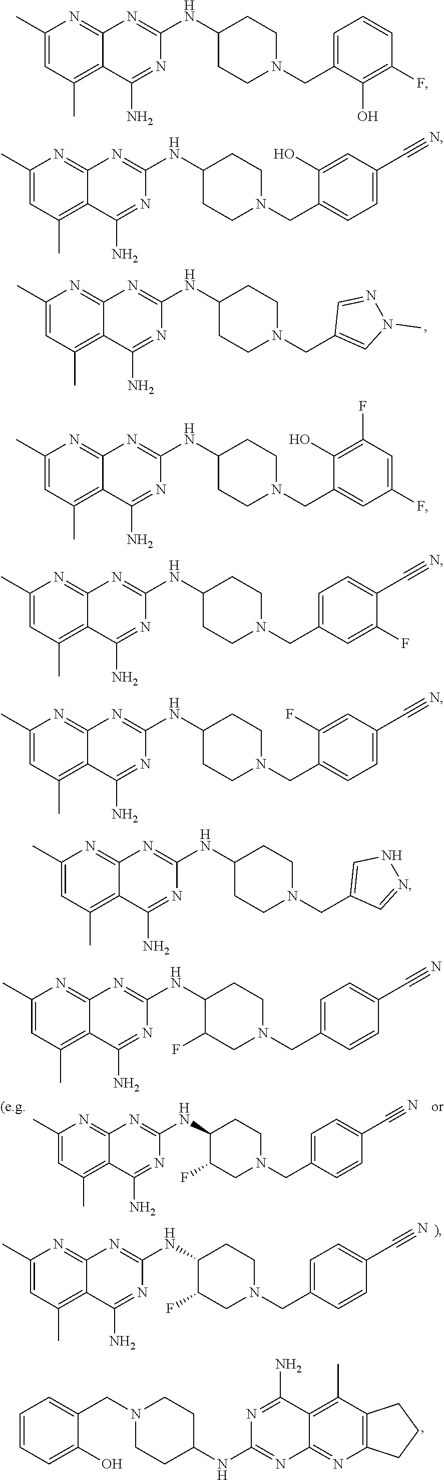

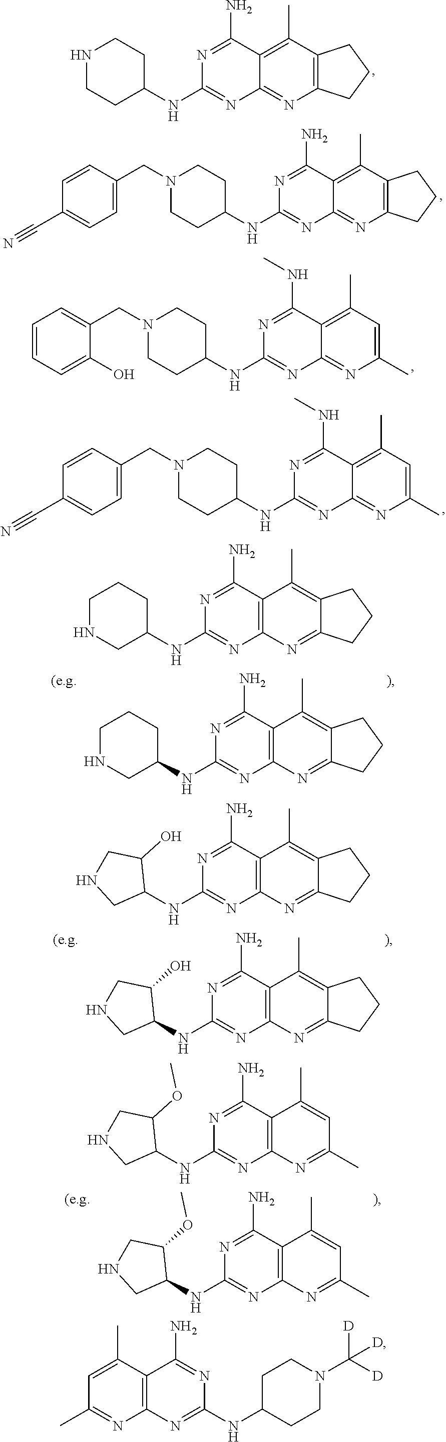

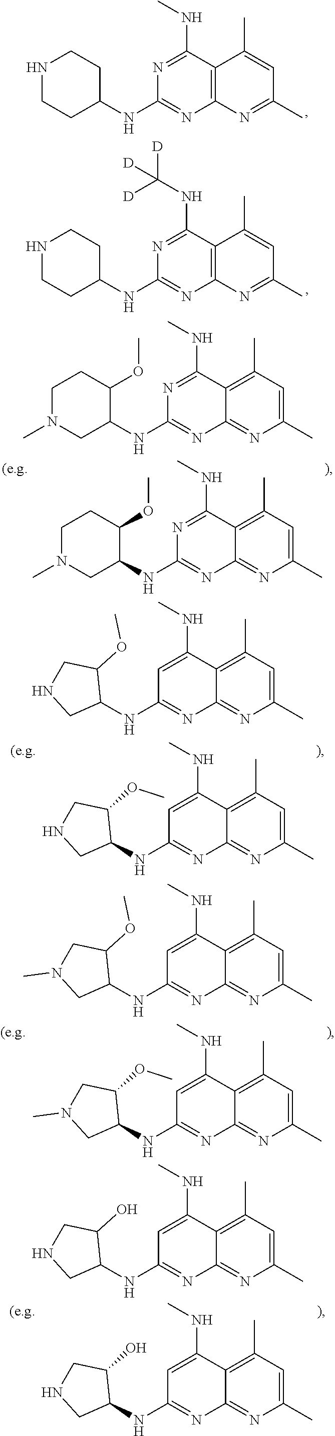

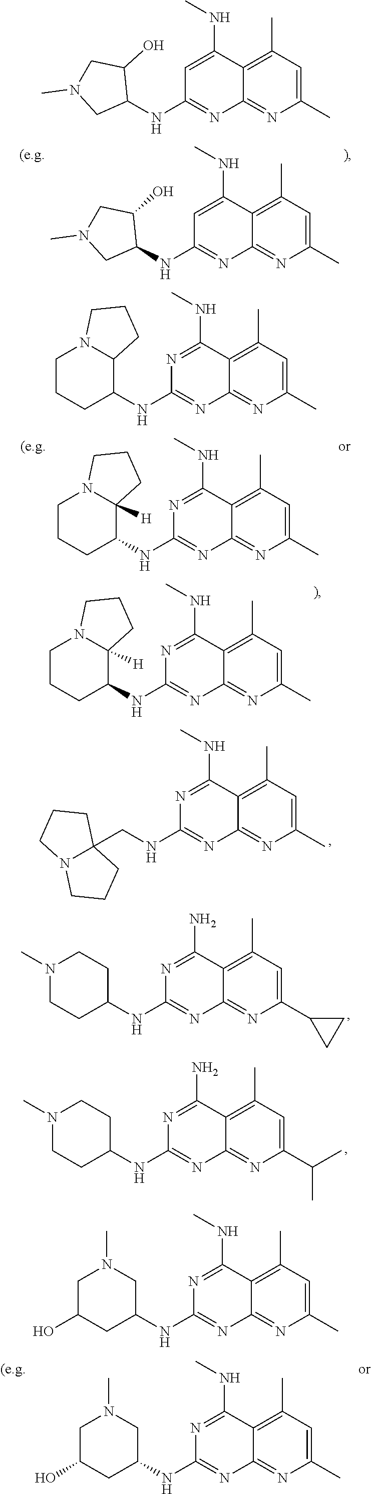

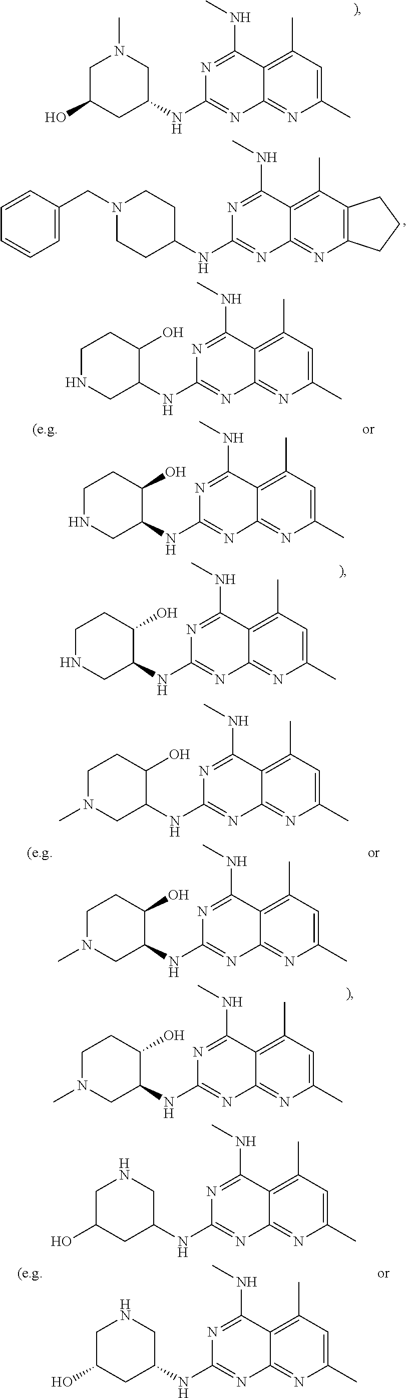

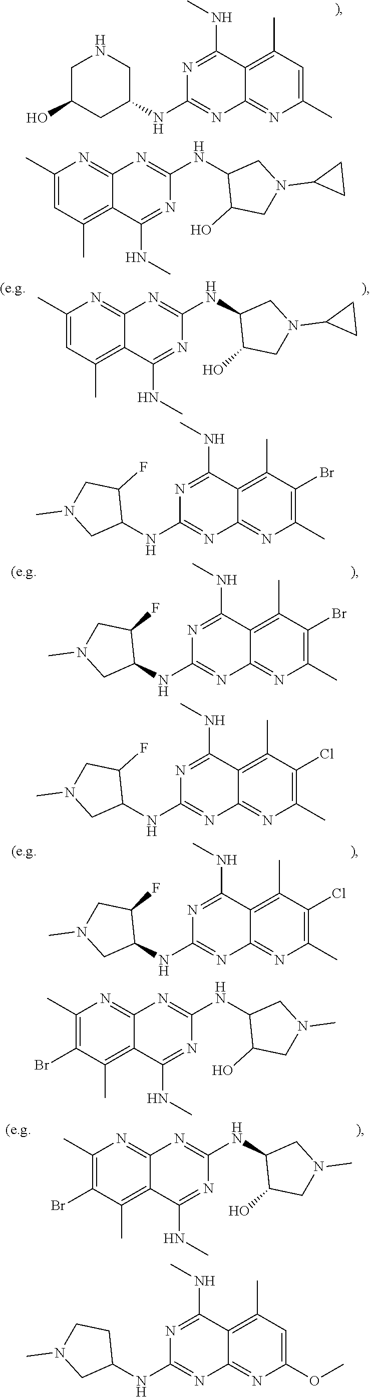

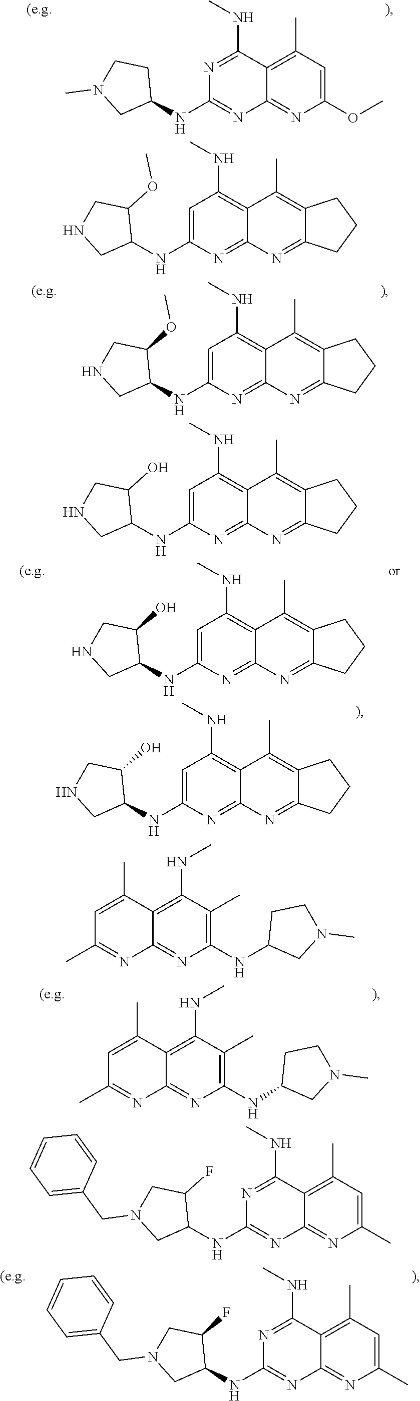

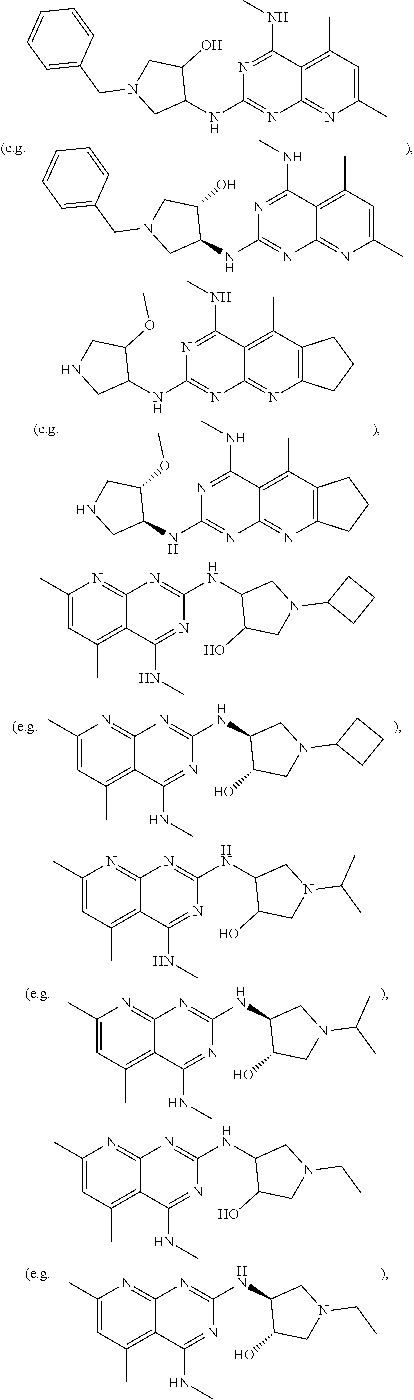

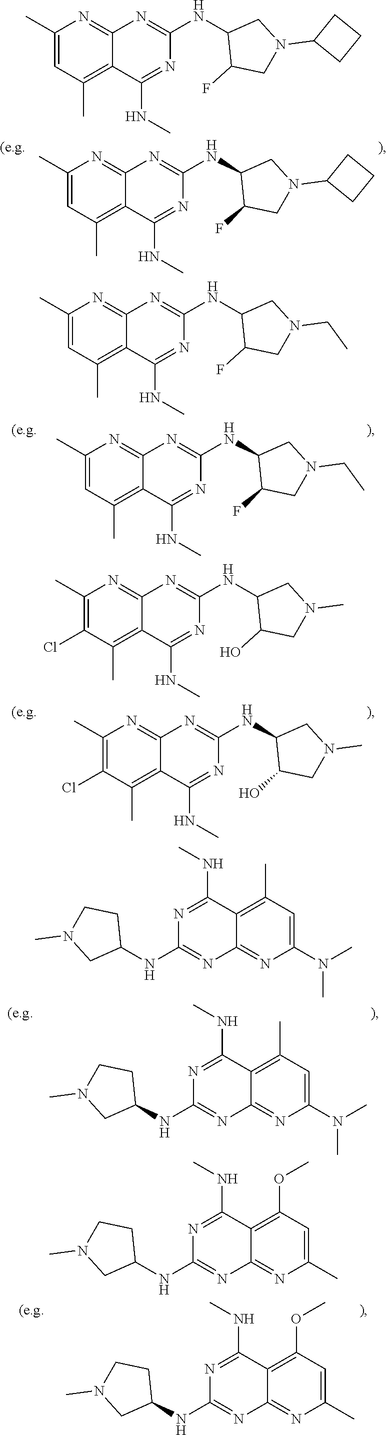

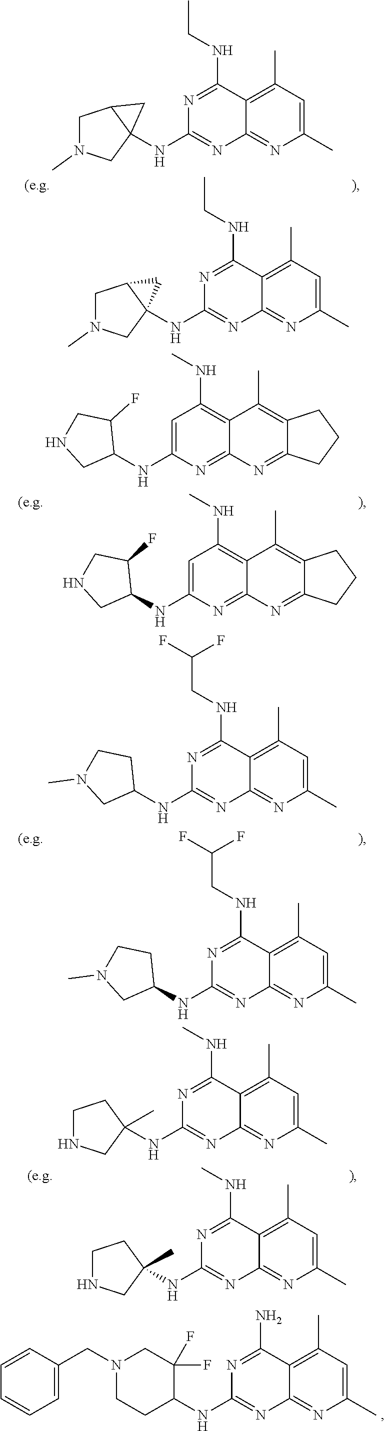

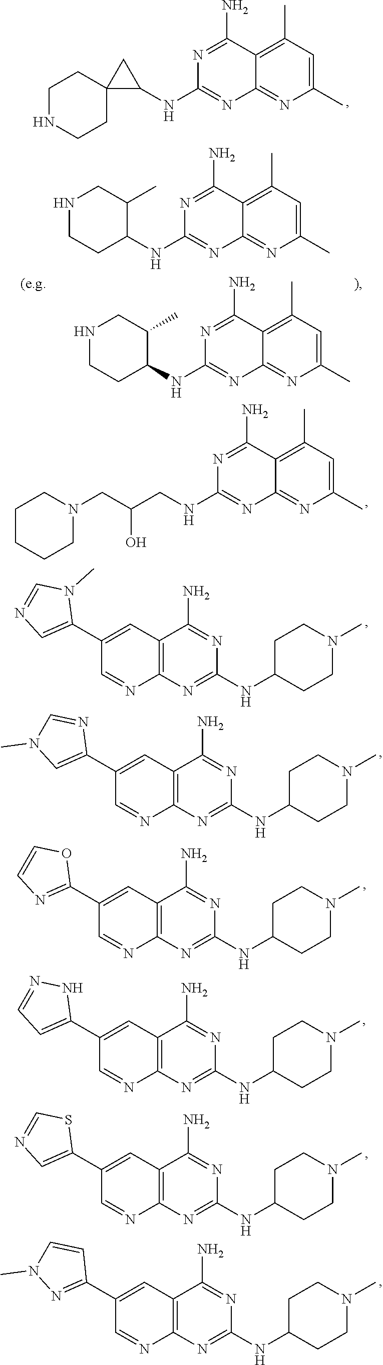

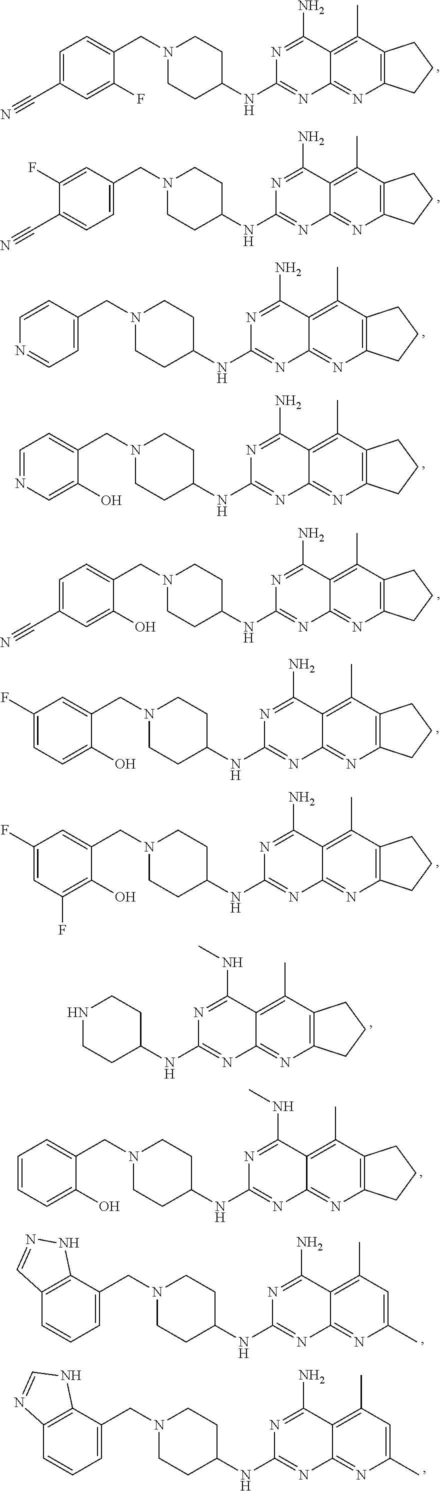

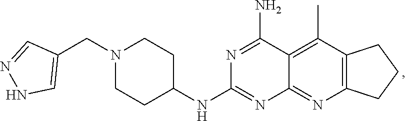

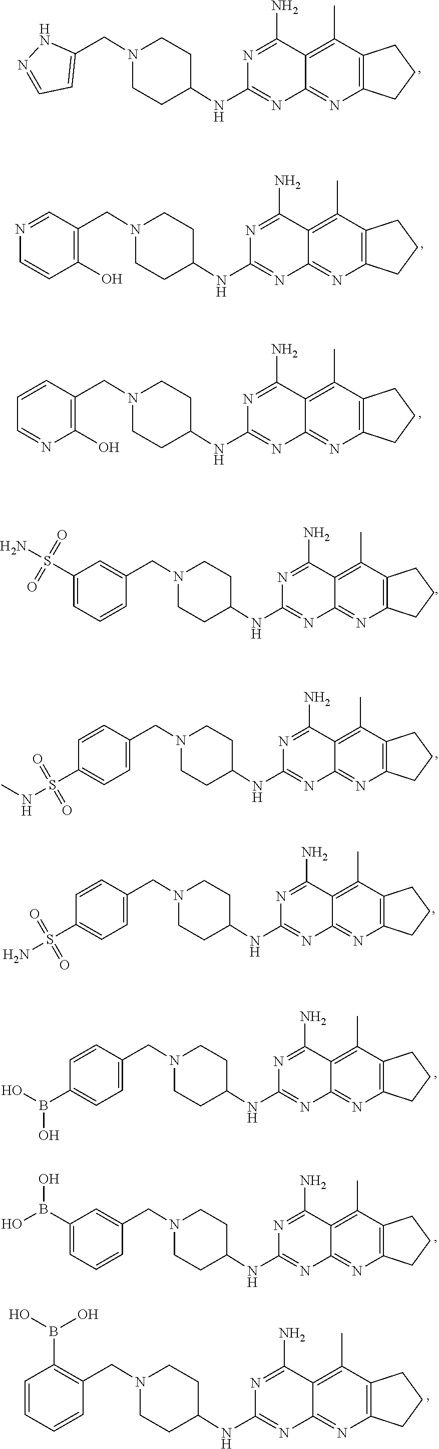

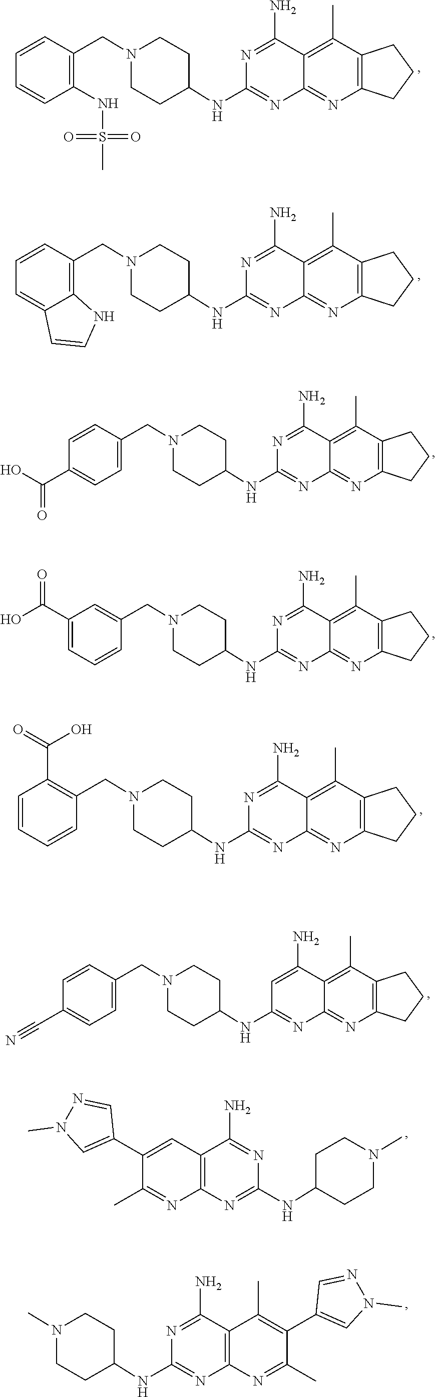

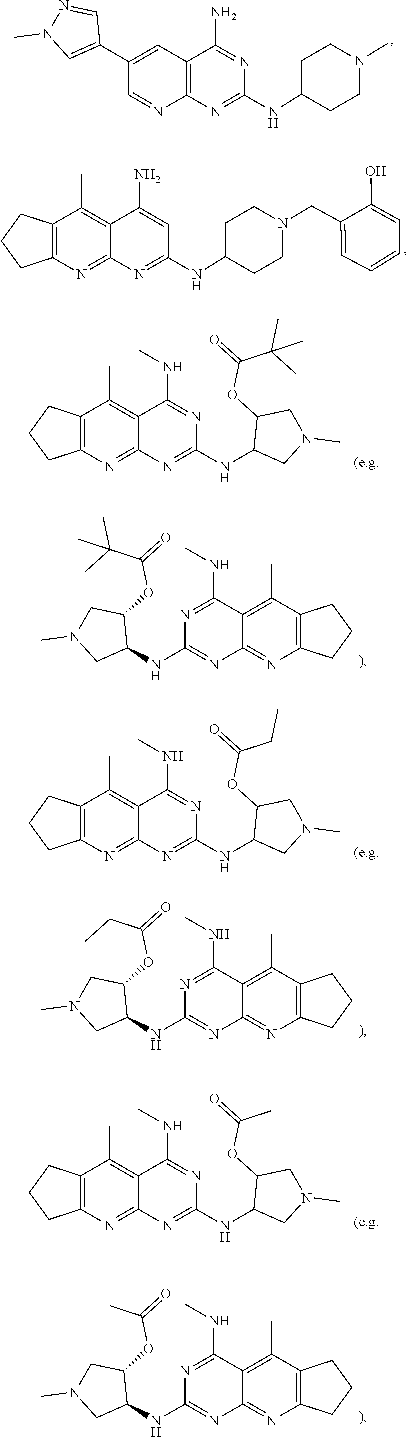

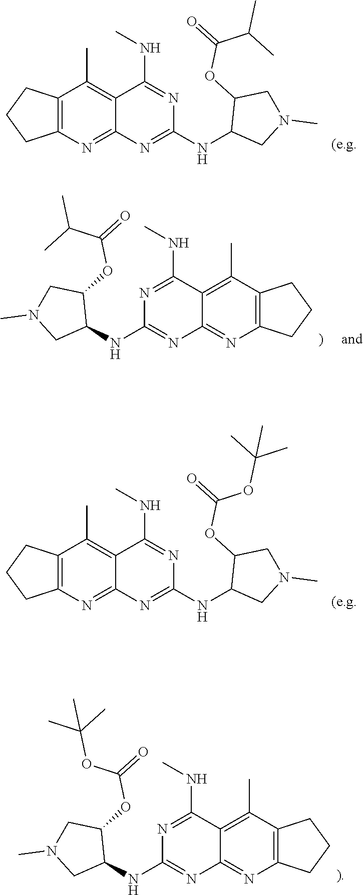

In accordance with a second aspect, the present invention provides a pharmaceutical composition comprising a compound defined in the first aspect, and one or more pharmaceutically acceptable excipients.

In accordance with a third aspect, the present invention provides a compound as defined in the first aspect or a pharmaceutical composition as defined in the second aspect, for use as a medicament.

In accordance with a fourth aspect, the present invention provides the use of a compound as defined in the first aspect or a pharmaceutical composition as defined in the second aspect, for the manufacture of a medicament.

In accordance with a fifth aspect, the present invention provides a compound as defined in the first aspect, or a pharmaceutical composition as defined in the second aspect, for use in a method of treatment or prophylaxis of a disease of the musculoskeletal system, a disease of the skin, a metabolic disease, a disease of the Nervous System, a Cardiovascular disease, an Endocrine disorder, a disease of the eye, a disease affecting the urogenital system, a haemic or lymphatic condition, a respiratory disease, an inflammatory or autoimmune condition, a disease of the Gastrointestinal system, a Neoplasm, cancer, or a disease or disorder selected from Amelogenesis Imperfecta, Anodontia, Odontodysplasia, Branchio-Oto-Renal Syndrome, Sotos Syndrome, and Waardenburg's Syndrome.

In accordance with a sixth aspect, the present invention provides a method for the treatment or prophylaxis of a disease of the musculoskeletal system, a disease of the skin, a metabolic disease, a disease of the Nervous System, a Cardiovascular disease, an Endocrine disorder, a disease of the eye, a disease affecting the urogenital system, a haemic or lymphatic condition, a respiratory disease, an inflammatory or autoimmune condition, a disease of the Gastrointestinal system, a Neoplasm, cancer, or a disease or disorder selected from Amelogenesis Imperfecta, Anodontia, Odontodysplasia, Branchio-Oto-Renal Syndrome, Sotos Syndrome, and Waardenburg's Syndrome, said method comprising administering to a subject, an effective amount of a compound as defined in the first aspect, or a pharmaceutical composition as defined in the second aspect.

In accordance with a seventh aspect, the present invention provides a compound as defined in the first aspect, or a pharmaceutical composition as defined in the second aspect, for use in a method of treatment or prophylaxis of a disease selected from: Recessive dystrophic epidermolysis bullosa, Junctional epidermolysis bullosa, Xeroderma pigmentosum, Netherton syndrome, Duchenne Muscular Dystrophy, Becker Muscular Dystrophy, Cystic Fibrosis, Alport syndrome, Dravet Syndrome, Aniridia, Methylmalonic Acidemia, Colorectal Cancer, Endometrium Cancer, Breast Cancer, Ovarian Cancer, Lung Squamous Cell Carcinoma, Head and Neck Squamous Cell Carcinoma, Familial adenomatous polyposis, Hemophilia A, Hemophilia B, Choroideremia, Pulmonary Artery Hypertension, Ataxia telangiectasia, Shwachman-Diamond syndrome, Mucopolysaccharidosis Type I, Mucopolysaccharidosis Type VI, Mucopolysaccharidosis type III, Niemann-Pick Disease, Primary Ciliary Dyskinesia, Usher syndrome, and Retinitis Pigmentosa.

In accordance with an eighth aspect, the present invention provides a method for the treatment or prophylaxis of a disease selected from: Recessive dystrophic epidermolysis bullosa, Junctional epidermolysis bullosa, Xeroderma pigmentosum, Netherton syndrome, Duchenne Muscular Dystrophy, Becker Muscular Dystrophy, Cystic Fibrosis, Alport syndrome, Dravet Syndrome, Aniridia, Methylmalonic Acidemia, Colorectal Cancer, Endometrium Cancer, Breast Cancer, Ovarian Cancer, Lung Squamous Cell Carcinoma, Head and Neck Squamous Cell Carcinoma, Familial adenomatous polyposis, Hemophilia A, Hemophilia B, Choroideremia, Pulmonary Artery Hypertension, Ataxia telangiectasia, Shwachman-Diamond syndrome, Mucopolysaccharidosis Type I, Mucopolysaccharidosis Type VI, Mucopolysaccharidosis type III, Niemann-Pick Disease, Primary Ciliary Dyskinesia, Usher syndrome, and Retinitis Pigmentosa.

In accordance with a ninth aspect, the present invention provides the use of a compound as defined in the first aspect, or a pharmaceutical composition as defined in the second aspect for the manufacture of a medicament for the treatment or prophylaxis of a disease of the musculoskeletal system, a disease of the skin, a metabolic disease, a disease of the Nervous System, a Cardiovascular disease, an Endocrine disorder, a disease of the eye, a disease affecting the urogenital system, a haemic or lymphatic condition, a respiratory disease, an inflammatory or autoimmune condition, a disease of the Gastrointestinal system, a Neoplasm, cancer, or a disease or disorder selected from Amelogenesis Imperfecta, Anodontia, Odontodysplasia, Branchio-Oto-Renal Syndrome, Sotos Syndrome, and Waardenburg's Syndrome.

In accordance with a tenth aspect, the present invention provides a method of allowing translational read-through of PTC mutations in a subject, said method comprising administering to a subject an effective amount of a compound as defined in the first aspect, or a pharmaceutical composition as defined in the second aspect.

In accordance with an eleventh aspect, the present invention provides a compound as defined in the first aspect, or a pharmaceutical composition as defined in the second aspect, for use in a method of treating conditions or disorders which are associated with a PTC mutation in a subject, said method comprising administering to a subject an effective amount of a compound as defined in the first aspect, or a pharmaceutical composition as defined in the second aspect.

In accordance with a twelfth aspect, the present invention provides a method of treating conditions or disorders which are associated with a PTC mutation in a subject, said method comprising administering to a subject an effective amount of a compound as defined in the first aspect, or a pharmaceutical composition as defined in the second aspect.

Suitably a PTC mutation may be any mutation which generates a premature termination codon (PTC) in a gene. Suitably a PTC mutation may be any mutation which generates an in-frame premature termination codon (PTC) in a gene. Suitably a PTC mutation occurs in the nucleotide sequence of a gene. Suitably a PTC mutation may occur in the coding region or non-coding region of a gene. Suitably a PTC mutation may occur in the coding region of a gene.

Suitably the mutation may be a point mutation, for example a substitution, deletion or insertion mutation. Suitably the mutation may be a substitution mutation. Suitably the substitution mutation may replace one nucleotide with another different nucleotide, suitably within the nucleotide sequence of a gene. Suitably the mutation may therefore be regarded as a nonsense mutation. Suitably the mutation may be a nonsense mutation which causes an in-frame PTC in a gene. Suitably the PTC may be any known termination codon such as TAG, TAA or TGA. Suitably the PTC may be TGA. Suitably therefore in some embodiments, the PTC mutation may be a substitution mutation which generates a PTC comprising TAG, TAA or TGA in a gene. Suitably therefore in some embodiments, the PTC mutation may be a substitution mutation of CGA to TGA or CAG to TAG in a gene.

A condition or disorder associated with a PTC mutation in a subject may be a condition or disorder which is directly or indirectly caused by a PTC mutation in a subject. Suitably a condition or disorder associated with a PTC mutation in a subject may be a condition or disorder which is directly caused by a PTC mutation in a subject. Suitably a condition or disorder associated with a PTC mutation in a subject may be a condition or disorder which is caused by a PTC mutation in one or more genes in a subject. Suitably a condition or disorder associated with a PTC mutation in a subject may be a condition or disorder which is directly caused by a PTC mutation in one or more genes in a subject. Suitably a condition or disorder associated with a PTC mutation in a subject may be a condition or disorder which is caused by a PTC mutation in one or more genes in a subject leading to a loss of function of the or each gene. Suitably reference herein to ‘a’ PTC mutation may be understood to refer to one or more PTC mutations, or a plurality of PTC mutations, suitably which may be present in one or more genes in a subject.

Suitably the one or more genes in which the PTC mutation is present are genes relating to one or more diseases as described herein. Suitably the one or more genes may be directly or indirectly linked with one or more diseases described herein. Suitably the one or more genes may contribute to, or cause, the phenotype of one or more diseases described herein. Such genes may be for example LAMB3, COL7A, COL4, CFTR, and DMD, which are each associated with one or more of the diseases identified herein. In some embodiments, for example in which the disease is cancer, the one or more genes in which the PTC mutation is present may be cell cycle genes, tumour suppressor genes etc. Such tumour suppressor genes may include, for example, TP53, PTEN, APC, ARID1A or CTCF. Suitably such mutations may be regarded as driver mutations, suitably which drive a disease phenotype, such as tumour growth. Therefore, in some embodiments, the PTC mutation may be regarded as a driver mutation. In other embodiments, the one or more genes in which the PTC mutation is present may not contribute to or cause the disease phenotype. Suitably such mutations may be regarded as bystander mutations. Therefore in some embodiments, the PTC mutation may be regarded as a bystander mutation.

Conditions or disorders associated with a PTC mutation are well known in the art, and suitable such disorders which may be treated or prevented by the present invention are described hereinbelow. However, suitably, such disorders that are associated with PTC mutations may also be identified or determined by the use of standard molecular biology techniques. Suitably a condition or disorder may be determined as being associated with a PTC mutation by the use of molecular biology techniques, such as sequencing, single strand conformational polymorphism, denaturing gradient gel electrophoresis, heteroduplex analysis, or restriction fragment length polymorphism. Suitably a PTC mutation present in one or more genes of a subject which may cause a disease or condition can be identified using such techniques. Suitably, whole genome sequencing or whole exome sequencing may be used to identify a PTC mutation in one or more genes of a subject, suitably which may cause a disease or condition. Suitable methodologies may be described in Karagiannakos et al. Cancers (Basel) 2022, 14, 664, Stark et al. ‘A prospective evaluation of whole-exome sequencing as a first-tier molecular test in infants with suspected monogenic disorders’ Genetics in Medicine, Volume 18, Issue 11, 2016, North et al. ‘Approach to the diagnosis of congenital myopathies’ Neuromuscular Disorders, Volume 24, Issue 2, 2014, for example.

Suitably such techniques may be applied to a sample obtained from a subject suffering from a disease or condition or suspected of suffering from a disease or condition, suitably which may be caused by a PTC mutation. Suitably such techniques may comprise comparing a result from said sample, to a result obtained from a reference sample. Suitably a reference sample may be a sample of the same type from a healthy subject, suitably from an equivalent healthy subject of the same age, nationality, race, height, weight etc. Suitable samples may include: a blood sample, serum sample, CNS fluid sample, tissue sample, cell sample, and the like. Suitably comparing a result in the context of the techniques above may comprise comparing the sequence of one or more genes from the subject suffering from a disease or condition to the sequence of the same one or more genes of the healthy subject. Suitably to identify PTC mutations within the one or more genes of the subject suffering from a disease or condition. Suitably such sequence comparisons may be carried out by available software, such as alignment software.

PTC read-through agents, such as the compounds disclosed herein, may in one or more embodiments, be of value and used in the treatment or amelioration of the following non-limiting examples of disorders and diseases. Suitably any of the following disorders and diseases may be regarded as a disease or disorder associated with a PTC mutation in a subject. Suitably therefore the present invention provides a method of treating conditions or disorders which are associated with PTC mutations in a subject, wherein the conditions or disorders are selected from any listed in the following paragraphs.

The disease may be a disease of the musculoskeletal system. The disease of the musculoskeletal system may be a disease selected from Shwachman-Diamond Syndrome, Rickets, Laron Syndrome, Muscular Dystrophies (e.g. Duchenne muscular dystrophy (DMD)), Microcephaly, congenital limb deformities, Muscle Spasticity, Dwarfism, Gigantism, Osteopoikilosis, Cleidocranial Dysplasia, Synostosis, Mitochondrial Myopathies and Encephalomyopathies, Craniosynostoses, Mandibulofacial Dysostosis, Scoliosis, Osteoporosis, Osteopetrosis, Hyperostosis, Osteosclerosis, Osteogenesis Imperfecta, Holoprosencephaly, Acromegaly, Arthrogryposis, and Campomelic Dysplasia.

The disease may be a disease of the skin. The disease of the skin may be a disease selected from Ectodermal Dysplasia, Epidermolysis Bullosa, Focal Dermal Hypoplasia, Ichthyosis Vulgaris, Autosomal Recessive Congenital Ichthyosis (ARCI), Recessive X-linked Ichthyosis, Lamellar Ichthyosis, Congenital Ichthyosiform Erythroderma, Harlequin Ichthyosis, Epidermolytic Ichthyosis, Superficial Epidermolytic Ichthyosis, CHILD syndrome, Netherton syndrome, MEDNIK syndrome, Neutral lipid storage disease with ichthyosis, Atopic Dermatitis, Alopecia, Atrichia with papular lesions, Hypotrichosis, Incontinentia Pigmenti, Epidermolysis Bullosa Simplex, Lipodystrophy, Hidradenitis Suppurativa, Hyperpigmentation, Junctional Epidermolysis Bullosa, Oculocutaneous Albinism, Chronic Mucocutaneous Candidiasis, Chronic Mucocutaneous Ichthyosis, X-Linked Hypopigmentation, Cutis Laxa, Menkes Kinky Hair Syndrome, Palmoplantar Keratoderma, Ehlers-Danlos Syndrome, Congenital Monilethrix, Onychomycosis, Benign Familial Pemphigus, Albinism; Kindler syndrome, Cowden Syndrome, Xeroderma pigmentosum, epidermodysplasia verruciformis, Lipoid proteinosis, dyschromatosis symmetrica hereditaria, Striate keratodermas, Autosomal Recessive Congenital Anonychia, Focal dermal hypoplasia, and Skin fragility/woolly hair syndrome.

The disease may be a metabolic disease. The metabolic disease may be selected from Congenital Adrenal Hyperplasia, Inborn errors of Amino Acid Metabolism, Hypertriglyceridemia, Diabetes Mellitus, Type 1 and Type 2, Glycogen Storage Disease, Optic Atrophy, Calcinosis, Multiple Sulfatase Deficiency Disease, Fabry Disease, Hyperinsulinism, Familial Hypophosphatemia, Pseudohypoaldosteronism, Tangier Disease, Amyotrophic Lateral Sclerosis, Lactic Acidosis, Familial Amyloidosis, Mucopolysaccharidosis, Anemia, Sandhoff Disease, Cytochrome-c Oxidase Deficiency, Pyruvate Dehydrogenase Complex Deficiency Disease, Hypoglycemia, Neuronal Ceroid-Lipofuscinoses, Severe Combined Immunodeficiency, Chronic Idiopathic Jaundice, Progeria, Hyponatremia, Wasting Syndrome, Cystinuria, Glycogen Storage Disease, Gaucher Disease, Hypolipoproteinemias, Oculocerebrorenal Syndrome, Smith-Lemli-Opitz Syndrome, Adrenoleukodystrophy, Ataxia Telangiectasia, Canavan Disease, Carbamoyl-Phosphate Synthase I Deficiency Disease, beta-Mannosidosis, and lysosomal storage diseases, wherein the lysosomal storage disease may be selected from Niemann-Pick Disease Mucopolysaccharidosis type 1 (Hurler syndrome), Mucopolysaccharidosis type 6, Mucopolysaccharidosis type 7, CLN1 disease, and CL3 disease.

The disease may be a disease of the Nervous System. The disease of the Nervous System may be selected from Rett Syndrome, Ataxias, Sensorineural Hearing Loss, Epilepsy, Charcot-Marie-Tooth Disease, Spinal Muscular Atrophy, Spastic Paraplegia, Hydrocephalus, Migraine with Aura, Chorea, Tremor, Usher Syndromes, De Lange Syndrome, Duane Retraction Syndrome, Dementia, Myoclonus, Hereditary Sensory and Autonomic Neuropathies, Intellectual Disability, X-linked Mental Retardation, Fragile X Syndrome, Hypopituitarism, Leukoencephalopathies, Dystonia, Congenital Pain Insensitivity, Tourette Syndrome, Alzheimers Disease, Parkinsons Disease, Angelman Syndrome, Apraxias, Cerebral Palsy, and Frontotemporal Dementia.

The disease may be a Cardiovascular disease. The cardiovascular disease may be selected from Coronary Disease, Ventricular Fibrillation, Telangiectasis, Kartagener Syndrome, Alagille Syndrome, Andersen Syndrome, Atrioventricular Block, and Card iomyopathies.

The disorder may be an Endocrine disorder. The Endocrine disorder may be selected from Hypogonadism, Goiter, Fetal Macrosomia, Thyroid Hormone Resistance Syndrome, Gonadal Dysgenesis, Hypoparathyroidism, Neurogenic Diabetes Insipidus, and Androgen-Insensitivity Syndrome.

The disease may be a disease of the eye. The disease of the eye may be selected from Leber syndrome, Hereditary Optic Atrophies, Ectopia Lentis, Coloboma, Aphakia, and Choroideremia.

The disease may be a disease affecting the urogenital system. The disease affecting the urogenital system may be selected from Hypospadias, Hydrops Fetalis, Interstitial Nephritis, Polycystic Kidney Diseases, Azoospermia, Alport Syndrome and Azoospermia.

The disease or disorder may be a haemic or lymphatic condition. The haemic or lymphatic condition may be selected from alpha- and beta-Thalassemia, Afibrinogenemia, Hemophagocytic Lymphohistiocytosis, Factor XI Deficiency, Hemophilia A, von Willebrand Diseases, Factor V Deficiency, Sideroblastic Anemia, Hereditary Elliptocytosis, Neutropenia, Chronic Granulomatous Disease, Hereditary Spherocytosis, Polycythemia, Hemophilia B, Factor VII Deficiency, Bernard-Soulier Syndrome, Dyserythropoietic Anemia, Hemolytic Anemia, Idiopathic Thrombocytopenic Purpura, Thrombasthenia, Factor XIII Deficiency, Hepatoerythropoietic Porphyria, and Acute Intermittent Porphyria.

The disease may be a respiratory disease. The respiratory disease may be selected from Cystic Fibrosis, Pulmonary Hypertension, Lipoid Proteinosis of Urbach and Wiethe, Newborn Respiratory Distress Syndrome, Chronic Obstructive Pulmonary Disease, Chronic Obstructive, Asthma, and Choanal Atresia.

The disease or disorder may be an inflammatory or autoimmune condition. The inflammatory or autoimmune condition may be selected from Immunologic Deficiency Syndromes, and Leukocyte-Adhesion Deficiency Syndrome.

The disease may be a disease of the Gastrointestinal system. Diseases of the gastrointestinal system may be selected from Chronic Hepatitis B, Colitis, Intestinal Polyposis, Inflammatory Bowel Diseases, Hirschsprung Disease, Exocrine Pancreatic Insufficiency, Crohn's Disease, and Cholestasis.

The disease or disorder may be a Neoplasm. The Neoplasm may be selected from Acute Myeloid Leukemia, Paraganglioma, Rhabdoid Tumor, Rhabdomyoma, Adenoid Cystic Carcinoma, Large Cell Carcinoma, Lobular Carcinoma, Skin Appendage Carcinoma, Squamous Cell Carcinoma, Alveolar Rhabdomyosarcoma, Neuroectodermal Tumors, Multiple Hamartoma Syndrome, Pheochromocytoma, Nevus, Osteosarcoma, Teratoma, and Adenoma.

The disease or disorder may be cancer. The cancer may be selected from acoustic neuroma, anal cancer, bladder cancer, Bowen's disease, brain cancer, breast cancer, carcinomas including basal cell carcinoma, bile duct carcinoma, bronchogenic carcinoma, choriocarcinoma, embryonal carcinoma, cystadenocarcinoma, epithelial carcinoma, medullary carcinoma, NUT midline carcinoma (NMC), papillary carcinoma, papillary adenocarcinomas, renal cell carcinoma, sebaceous gland carcinoma, small cell lung carcinoma, squamous cell carcinoma, and sweat gland carcinoma, cervical cancer, chordoma, colon cancer, colorectal cancer, craniopharyngioma, dysproliferative changes (dysplasias and metaplasias), endometrial cancer, ependymoma, esophageal cancer, essential thrombocythemia, estrogen-receptor positive breast cancer, Ewing's tumour, genital cancer, cancer of the cervix, cancer of the vulva, vulvar intraepithelial neoplasia (VIN), cancer of the vagina, germ cell testicular cancer, gastrointestinal cancers, gastric cancer, glioblastoma, glioma, heavy chain disease, hemangioblastoma, hepatocellular cancer, hepatoma, hormone insensitive prostate cancer, keratinocyte carcinomas, kidney cancer, leukaemias including acute leukaemia, acute lymphocytic leukaemia, acute myeloid leukaemia, acute myelocytic leukaemia (monocyctic, myeloblastic, adenocarcinoma, angiosarcoma, astrocytoma, myelomonocytic and promyelocytic), acute t-cell leukaemia, chronic leukaemia, chronic lymphocytic leukaemia, chronic myelocytic (granulocytic) leukaemia, chronic myelogenous leukaemia, erythroleukemia, lymphoblastic leukaemia, and myelogenous leukaemia, liver cancer, lung cancer, lymphoid malignancies of T-cell or B-cell origin, lymphomas (Hodgkin's and non-Hodgkin's) including cutaneous T-cell lymphoma, diffuse large B-cell lymphoma, and follicular lymphoma, cutaneous (skin) lymphomas, malignancies and hyperproliferative disorders including of the bladder, breast, colon, lung, ovaries, pancreas, prostate, skin and uterus, advanced malignancies, medulloblastoma, melanoma, meningioma, Merkel cell cancer mesothelioma, metastatic cancer, multiple myeloma, myeloma, pancreatic cancer, myelofibrosis, myeloproliferative neoplasms, neuroblastoma, non-small cell lung cancer, head and neck cancer, oligodendroglioma, oral cancer, ovarian cancer, pancreatic cancer, pinealoma, polycythemia vera, prostate cancer, rectal cancer, retinoblastoma, sarcomas including chondrosarcoma, endotheliosarcoma, fibrosarcoma, gliosarcoma, leiomyosarcoma, liposarcoma, lymphagioendotheliosarcoma, lymphangiosarcoma, myxosarcoma, Castleman's disease and Kaposi's sarcoma, osteogenic sarcoma, and rhabdomyosarcoma, seminoma, skin cancer, skin adnexal tumors, and sarcomas, small cell lung cancer, solid tumors, stomach cancer, synovioma, testicular tumours, thyroid cancer, uterine cancer, Waldenstrom's macroglobulinemia, and Wilms' tumour.

The disease or disorder may be selected from Amelogenesis Imperfecta, Anodontia, Odontodysplasia, Branchio-Oto-Renal Syndrome, Sotos Syndrome, and Waardenburg's Syndrome.

The disease or disorder may be selected from: Recessive dystrophic epidermolysis bullosa, Junctional epidermolysis bullosa, Xeroderma pigmentosum, Netherton syndrome, Duchenne Muscular Dystrophy, Becker Muscular Dystrophy, Cystic Fibrosis, Dravet Syndrome, Aniridia, Methylmalonic Acidemia, Colorectal Cancer, Endometrium Cancer, Breast Cancer, Ovarian Cancer, Lung Squamous Cell Carcinoma, Head and Neck Squamous Cell Carcinoma, Familial adenomatous polyposis, Hemophilia A, Hemophilia B, Choroideremia, Pulmonary Artery Hypertension, Ataxia telangiectasia, Shwachman-Diamond syndrome, Mucopolysaccharidosis Type I, Mucopolysaccharidosis Type VI, Mucopolysaccharidosis type III, Niemann-Pick Disease, Primary Ciliary Dyskinesia, and ciliopathies (such as Usher syndrome or Retinitis Pigmentosa).

The disease or disorder may be selected from: Recessive dystrophic epidermolysis bullosa, Junctional epidermolysis bullosa, Duchenne Muscular Dystrophy, Cystic Fibrosis, Colorectal Cancer, Endometrium Cancer, Breast Cancer, Ovarian Cancer, Lung Squamous Cell Carcinoma, Head and Neck Squamous Cell Carcinoma and Familial adenomatous polyposis. Suitably such disorders may be associated with a PTC mutation in a subject. Suitably for example, the disease may be Junctional epidermolysis bullosa, associated with a PTC mutation in the LAMB3 gene. Suitably for example, the disease may be Recessive dystrophic epidermolysis bullosa, associated with a PTC mutation in the COL7A1 gene. Suitably for example, the disease may be Duchenne Muscular Dystrophy, associated with a PTC mutation in the DMD gene. Suitably for example, the disease may be Cystic Fibrosis associated with a PTC mutation in the CTFR gene. Suitably for example, the disease may be Familial adenomatous polyposis associated with a PTC mutation in the APC gene. Suitably for example, the disease may be a cancer as listed above associated with a PTC mutation in a gene selected from: TP53, PTEN, ARID1A or CTCF.

Suitably therefore the present invention provides a method of treating conditions or disorders which are associated with PTC mutations in a subject, wherein the condition or disorder associated with PTC mutations in a subject is selected from recessive dystrophic epidermolysis bullosa, Junctional epidermolysis bullosa, Xeroderma pigmentosum, Netherton syndrome, Duchenne Muscular Dystrophy, Becker Muscular Dystrophy, Cystic Fibrosis, Dravet Syndrome, Aniridia, Methylmalonic Acidemia, Familial adenomatous polyposis, Hemophilia A, Hemophilia B, Choroideremia, Pulmonary Artery Hypertension, Ataxia telangiectasia, Shwachman-Diamond syndrome, Mucopolysaccharidosis Type I, Mucopolysaccharidosis Type VI, Mucopolysaccharidosis type III, Niemann-Pick Disease, Primary Ciliary Dyskinesia, Ciliopathies (such as Usher syndrome or Retinitis Pigmentosa) and Alport Syndrome. In some embodiments, the present invention provides a method of treating conditions or disorders which are associated with PTC mutations in a subject, wherein the condition or disorder associated with PTC mutations in a subject is selected from recessive dystrophic epidermolysis bullosa, Junctional epidermolysis bullosa, Duchenne Muscular Dystrophy, Cystic Fibrosis, and Familial adenomatous polyposis.

PTC read-through agents, such as the compounds disclosed herein, may in one or more embodiments, also be of value and used in the palliation, diagnosis or prevention of any disease, disorder or condition in humans of one or more of the aforesaid non-limiting examples of disorders and diseases. Suitably any of the aforesaid disorders and diseases may be regarded as a disease or disorder associated with a PTC mutation in a subject. Suitably therefore the present invention further provides a method of palliation, diagnosis or prevention of conditions or disorders which are associated with PTC mutations in a subject, wherein the conditions or disorders are selected from any listed in the aforesaid paragraphs.

Treatment or amelioration with PTC read-through agents, such as compositions comprising the compounds disclosed herein or salts thereof (or combinations thereof), in some embodiments may be effective if administered orally. In some other embodiments may be effective if applied topically, and in some further embodiments may be effective if applied topically and orally.

In some embodiments, compositions comprising a novel compound disclosed herein or salt thereof (or combinations thereof) may be administered to young children. In some embodiments, compositions comprising a compound of the invention or salt thereof (or combinations thereof) may be administered to adolescents or teenagers. In some embodiments, compositions comprising a compound of the invention or salt thereof (or combinations thereof) may be administered to adults.

The disclosure may also be defined according to any one of the following numbered clauses:
1. A compound of formula (I), or a tautomeric form thereof, or a pharmaceutically acceptable salt or N-oxide thereof:

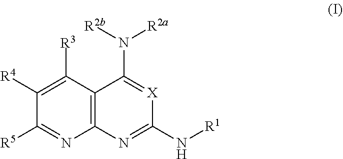

wherein
X is N or CR⁶;
R¹ is independently selected from C₀-C₆-alkylene-R^{1a}, and C₂-C₆-alkylene-R^{1b};
R^{1a} is independently selected from C₃-C₈ cycloalkyl, C₅-C₈ cycloalkenyl, 3- to 10-membered heterocycloalkyl, and 5- to 10-membered heterocycloalkenyl; wherein R^{1a} is optionally substituted with from 1 to 4 R⁹ groups; wherein where R^{1a} is heterocycloalkyl or heterocycloalkenyl, the heterocycloalkyl or heterocycloalkenyl is optionally fused to a phenyl ring, wherein the phenyl ring is optionally substituted with from 1 to 4 R¹⁰ groups;
R^{1b} is independently selected from NR^{7a}R^{8a} and OR⁷;
   wherein R¹ comprises at least one nitrogen atom;
R^{2a} is independently at each occurrence selected from H and C₁-C₄ alkyl;
R^{2b} is independently at each occurrence selected from H, C₁-C₄ alkyl, C₀-C₄ alkyl-R^{2c}, C(O)—C₁-C₄-alkyl, S(O)—C₁-C₄-alkyl, and S(O)₂—C₁-C₄-alkyl;
R^{2c} is independently selected from C₃-C₈ cycloalkyl, C₅-C₈ cycloalkenyl, phenyl, 3- to 8-membered heterocycloalkyl, 5- to 8-membered heterocycloalkenyl and 5-, or 6-membered heteroaryl; wherein where R^{2c} is cycloalkyl, cycloalkenyl, heterocycloalkyl or heterocycloalkenyl, R^{2c} is optionally substituted with from 1 to 4 R⁹ groups; and where R^{2c} is phenyl, or heteroaryl, R^{5c} is optionally substituted with from 1 to 5 R¹⁰ groups;
   or R^{2a} and R^{2b}, together with the nitrogen atom to which they are attached, form a 5- to 8-membered heterocycloalkyl ring; optionally substituted with 1 to 4 R⁹ groups;
R³ is independently selected from H, cyano, C₁-C₄-alkylene-NR⁷R⁸, NR⁷R⁸, C₁-C₄-alkylene-OR⁷, OR⁷, SR⁷, SOR⁷, S(O)₂R⁷, S(O)₂NR⁷R⁷, CO₂R⁷, C(O)R⁷, C(O)NR⁷R⁷, C₁-C₄-alkyl, C₂-C₄-alkenyl, C₂-C₄-alkynyl, C₁-C₄-haloalkyl, NR⁷—C₀-C₄-alkylene-R^{3c}, O—C₀-C₄alkylene-R^{3c}, and C₀-C₄-alkylene-R^{3c};
R^{3c} is independently selected from C₃-C₈ cycloalkyl, C₅-C₈ cycloalkenyl, phenyl, 3- to 10-membered heterocycloalkyl, 5- to 10-membered heterocycloalkenyl and 5-, or 6-membered heteroaryl; wherein where R^{3c} is cycloalkyl, cycloalkenyl, heterocycloalkyl or heterocycloalkenyl, R^{3c} is optionally substituted with from 1 to 4 R⁹ groups; and where R^{3c} is phenyl or heteroaryl, R^{3c} is optionally substituted with from 1 to 5 R¹⁰ groups;
R⁴ is independently selected from H, halo, cyano, NR^{7b}R⁸, OR⁷, SR⁷, SOR⁷, S(O)₂R⁷, S(O)₂NR⁷R⁷, CO₂R⁷, C(O)R⁷, C(O)NR⁷R⁷, C₁-C₄-alkyl, C₂-C₄-alkenyl, C₂-C₄-alkynyl, C₁-C₄-haloalkyl, O—C₀-C₄-alkylene-R^{4c}, and C₀-C₄-alkylene-R^{4c};
R^{4c} is independently selected from C₃-C₈ cycloalkyl, C₅-C₈ cycloalkenyl, 3- to 10-membered heterocycloalkyl, 5- to 10-membered heterocycloalkenyl and 5-membered heteroaryl; wherein where R^{4c} is cycloalkyl, cycloalkenyl, heterocycloalkyl or heterocycloalkenyl, R^{4c} is optionally substituted with from 1 to 4 R⁹ groups; and where R^{4c} is heteroaryl, R^{4c} is optionally substituted with from 1 to 5 R¹⁰ groups;
R⁵ is independently selected from H, halo, cyano, C₁-C₄-alkylene-NR⁷R⁸, NR^{7b}R⁸, C₁-C₄-alkylene-OR⁷, OR^{7b}, SR⁷, SOR⁷, S(O)₂R⁷, S(O)₂NR⁷R⁷, CO₂R⁷, C(O)R⁷, C(O)NR⁷R⁷, C₁-C₄-alkyl, C₂-C₄-alkenyl, C₂-C₄-alkynyl, C₁-C₄-haloalkyl, NR⁷—C₀-C₄-alkylene-R^{5c}, O—C₀-C₄-alkylene-R^{5c}, and C₀-C₄-alkylene-R^{5c};
R^{5c} is independently selected from C₃-C₈ cycloalkyl, C₅-C₈ cycloalkenyl, phenyl, 3- to 10-membered heterocycloalkyl, 5- to 10-membered heterocycloalkenyl and 5-, or 6-membered heteroaryl; wherein where R^{5c} is cycloalkyl, cycloalkenyl, heterocycloalkyl or heterocycloalkenyl, R^{5c} is optionally substituted with from 1 to 4 R⁹ groups; and where R^{5c} is phenyl or heteroaryl, R^{5c} is optionally substituted with from 1 to 5 R¹⁰ groups;
   or R³ and R⁴ together with the carbon atoms to which they are attached form a ring selected from: phenyl, C₅-C₇-cycloalkyl, 5- to 7-membered heterocycloalkyl and 5- or 6-membered heteroaryl; wherein where the ring is cycloalkyl or heterocycloalkyl, it is optionally substituted with from 1 to 4 R⁹ groups and where the ring is phenyl or heteroaryl, it is optionally substituted with from 1 to 4 R¹⁰ groups;
   or R⁴ and R⁵ together with the carbon atoms to which they are attached form a ring selected from: phenyl, C₅-C₇-cycloalkyl, 5- to 7-membered heterocycloalkyl and 5- or 6-membered heteroaryl; wherein where the ring is cycloalkyl or heterocycloalkyl, it is optionally substituted with from 1 to 4 R⁹ groups and where the ring is phenyl or heteroaryl, it is optionally substituted with from 1 to 4 R¹⁰ groups;
R⁶ is independently selected from H, halo, C₁-C₆-alkyl, and C₁-C₆-haloalkyl;
R⁷ and R^{7a} are each independently at each occurrence selected from H and C₁-C₄ alkyl;
R^{7b} is independently at each occurrence selected from C₁-C₄ alkyl;
R⁸ is independently at each occurrence selected from H, C₁-C₄ alkyl, C₁-C₄-haloalkyl and C(O)—C₁-C₄-alkyl
R^{8a} is independently at each occurrence selected from H, C₁-C₄ alkyl, C₁-C₄-haloalkyl, C(O)—C₁-C₄-alkyl and phenyl optionally substituted with from 1 to 5 R¹⁰ groups;
   or R^{7a} and R^{8a}, together with the nitrogen atom to which they are attached, form a 5- to 8-membered heterocycloalkyl ring; optionally substituted with 1 to 4 R⁹ groups;
R⁹ is independently at each occurrence selected from ═O, ═S, halo, nitro, cyano, NR⁷R⁸, OR⁷, SR⁷, SOR⁷, S(O)₂R⁷, SO₂NR⁷R⁷, CO₂R⁷, C(O)R⁷, CONR⁷R⁷, C₁-C₄-alkyl, C₂-C₄-alkenyl, C₂-C₄-alkynyl, C₁-C₄-haloalkyl, C₃-C₈-cycloalkyl, C₁-C₃-alkylene-NR⁷R⁸, and C₀-C₃-alkylene-phenyl;
R¹⁰ is independently at each occurrence selected from halo, nitro, cyano, NR⁷R⁸, OR⁷, SR⁷, SOR⁷, S(O)₂R⁷, SO₂NR⁷R⁷, CO₂R⁷, C(O)R⁷, CONR⁷R⁷, C₁-C₄-alkyl, C₂-C₄-alkenyl, C₂-C₄-alkynyl, C₁-C₄-haloalkyl, C₃-C₅-cycloalkyl, C₁-C₃-alkylene-NR⁷R⁸, and C₁-C₃-alkylene-OR⁷; and
wherein any of the aforementioned alkyl, alkylene, alkenyl, or cycloalkyl groups is optionally substituted, where chemically possible, by 1 to 5 substituents which are each independently at each occurrence selected from the group consisting of: C₁-C₄-alkyl, oxo, halo, nitro, cyano, NR^{a}R^{b}, OR^{a}, SR^{a}, CO₂R^{a}, C(O)R^{a}, CONR^{a}R^{a}, S(O)R^{a}, and S(O)₂R^{a}; wherein R^{a} is independently at each occurrence selected from H, and C₁-C₄-alkyl; and R^{b} is independently at each occurrence selected from H, C₁-C₄-alkyl, C(O)—C₁-C₄-alkyl and S(O)₂—C₁-C₄-alkyl.
2. A compound according to clause 1, wherein X is N.
3. A compound according to clause 1 or clause 2, wherein R^{2a} and R^{2b} are each H.
4. A compound according to any one of clauses 1 to 3, wherein R¹ comprises at least one amine nitrogen.
5. A compound according to any one of clauses 1 to 4, wherein R¹ is C₀-C₆-alkylene-R^{1a}.
6. A compound according to clause 5, wherein R¹ is R^{1a}.
7. A compound according to clause 5 or clause 6, wherein Ria is a 3- to 10-membered heterocycloalkyl group having a nitrogen in the ring system.
8. A compound according to any one of clauses 1 to 7, wherein R³ is H.
9. A compound according to any one of clauses 1 to 7, wherein R³ is C₁-C₄-alkyl.
10. A compound according to any one of clauses 1 to 9, wherein R⁴ is H.
11. A compound according to any one of clauses 1 to 9, wherein R⁴ is C₁-C₄-alkyl.
12. A compound according to any one of clauses 1 to 11, wherein R⁵ is R^{5C}.
13. A compound according to any one of clauses 1 to 12, wherein R^{5c} is phenyl optionally substituted with from 1 to 5 R¹⁰ groups.
14. A compound according to any one of clauses 1 to 12, wherein R^{5c} is 6-membered heteroaryl optionally substituted with from 1 to 5 R¹⁰ groups.
15. A compound according to any one of clauses 1 to 11, wherein R⁵ is H.
16. A compound according to any one of clauses 1 to 11, wherein R⁵ is C₁-C₄-alkyl.
17. A pharmaceutical composition comprising a compound according to any one of clauses 1 to 16, and one or more pharmaceutically acceptable excipients.
18. A compound according to any one of clauses 1 to 16, or a pharmaceutical composition according to clause 17, for use as a medicament.
19. A compound according to any one of clauses 1 to 16, or a pharmaceutical composition according to clause 17, for use in the treatment of: a disease of the musculoskeletal system, a disease of the skin, a metabolic disease, a disease of the Nervous System, a Cardiovascular disease, an Endocrine disorder, a disease of the eye, a disease affecting the urogenital system, a haemic or lymphatic condition, a respiratory disease, an inflammatory or autoimmune condition, a disease of the Gastrointestinal system, a Neoplasm, cancer, or a disease or disorder selected from Amelogenesis Imperfecta, Anodontia, Odontodysplasia, Branchio-Oto-Renal Syndrome, Sotos Syndrome, and Waardenburg's Syndrome.
20. A compound according to any one of clauses 1 to 16, or a pharmaceutical composition according to clause 17, for use in the treatment of a disease selected from: Recessive dystrophic epidermolysis bullosa, Junctional epidermolysis bullosa, Xeroderma pigmentosum, Netherton syndrome, Duchenne Muscular Dystrophy, Becker Muscular Dystrophy, Cystic Fibrosis, Dravet Syndrome, Aniridia, Methylmalonic Acidemia, Colorectal Cancer, Endometrium Cancer, Breast Cancer, Ovarian Cancer, Lung Squamous Cell Carcinoma, Head and Neck Squamous Cell Carcinoma, Familial adenomatous polyposis, Hemophilia A, Hemophilia B, Choroideremia, Pulmonary Artery Hypertension, Ataxia telangiectasia, Shwachman-Diamond syndrome, Mucopolysaccharidosis Type I, Mucopolysaccharidosis Type VI, Mucopolysaccharidosis type III, Niemann-Pick Disease, and Primary Ciliary Dyskinesia.
21. A compound according to any one of clauses 1 to 16, or a pharmaceutical composition according to clause 17, for use in a method of treating conditions or disorders which are associated with a PTC mutation in a subject.

### BRIEF DESCRIPTION OF THE DRAWINGS

Embodiments of the invention are further described hereinafter with reference to the accompanying drawings, in which:

FIG. 1 shows the effect on truncated and full-length (FL) p53 expression upon treatment of HDQ-P1 cells with Example 5 (0.03-80 μM) and also the effect on p21 expression upon treatment of HDQ-P1 cells with Example 5 (33 μM) compared to DMSO (0.3%), and G418 (200 μM).

FIG. 2 shows the effect of the treatment of Col7a1 R578X transiently transfected HEK293 cells with Example 5 (10-50 μM) compared to DMSO (1%), and G418 (200 μM). These data are normalised to the effect of G418 (200 μM).

FIG. 3 shows the effect of the treatment of Col7a1 R137X stably transfected HEK293 cells with Example 5 (10-30 μM) compared to DMSO (1%), and G418 (200 μM). These data are normalised to the effect of G418 (200 μM).

### DETAILED DESCRIPTION

The term Cₘ-Cₙ refers to a group with m to n carbon atoms. For the absence of doubt, the term “C₀” refers to a group with 0 carbon atoms.

The term “alkyl” refers to a monovalent linear or branched saturated hydrocarbon chain. For example, C₁-C₆-alkyl may refer to methyl, ethyl, n-propyl, iso-propyl, n-butyl, sec-butyl, tert-butyl, n-pentyl and n-hexyl. The alkyl groups may be unsubstituted or substituted by one or more substituents.

The term “alkylene” refers to a bivalent linear saturated hydrocarbon chain. For example, C₁-C₃-alkylene may refer to methylene, ethylene or propylene. The alkylene groups may be unsubstituted or substituted by one or more substituents. For the absence of doubt, the term “C₀-alkylene” refers to a group in which an alkylene chain is absent. For example, “C₀-alkylene-R^{a}” refers to an R^{a}.

The term “haloalkyl” refers to a hydrocarbon chain substituted with at least one halogen atom independently chosen at each occurrence from: fluorine, chlorine, bromine and iodine. The halogen atom may be present at any position on the hydrocarbon chain. For example, C₁-C₆-haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A haloalkyl group may be a fluoroalkyl group, i.e. a hydrocarbon chain substituted with at least one fluorine atom. Thus, a haloalkyl group may have any amount of halogen substituents. The group may contain a single halogen substituent, it may have two or three halogen substituents, or it may be saturated with halogen substituents.

The term “alkenyl” refers to a branched or linear hydrocarbon chain containing at least one double bond. The double bond(s) may be present as the E or Z isomer. The double bond may be at any possible position of the hydrocarbon chain. For example, “C₂-C₆-alkenyl” may refer to ethenyl, propenyl, butenyl, butadienyl, pentenyl, pentadienyl, hexenyl and hexadienyl. The alkenyl groups may be unsubstituted or substituted by one or more substituents.

The term “alkynyl” refers to a branched or linear hydrocarbon chain containing at least one triple bond. The triple bond may be at any possible position of the hydrocarbon chain. For example, “C₂-C₆-alkynyl” may refer to ethynyl, propynyl, butynyl, pentynyl and hexynyl. The alkynyl groups may be unsubstituted or substituted by one or more substituents.

The term “cycloalkyl” refers to a saturated hydrocarbon ring system containing 3, 4, 5 or 6 carbon atoms. For example, “C₃-C₆-cycloalkyl” may refer to cyclopropyl, cyclobutyl, cyclopentyl, cyclohexyl. The cycloalkyl groups may be unsubstituted or substituted by one or more substituents.

The term “y- to z-membered heterocycloalkyl” refers to a y- to z-membered heterocycloalkyl group. Thus it may refer to a monocyclic or bicyclic saturated group having from y to z atoms in the ring system and comprising 1 or 2 heteroatoms independently selected from O, S and N in the ring system (in other words 1 or 2 of the atoms forming the ring system are selected from O, S and N). Examples of heterocycloalkyl groups include; piperidine, piperazine, morpholine, thiomorpholine, pyrrolidine, tetrahydrofuran, tetrahydrothiophene, dihydrofuran, tetrahydropyran, dihydropyran, dioxane, azepine. A heterocycloalkyl group may be unsubstituted or substituted by one or more substituents. Heterocycloalkyl groups may be monocyclic. Heterocycloalkyl groups may be bicyclic. Bicyclic heterocycloalkyl groups may be fused, spirofused or bridged groups. The term “heterocycloalkyl fused to a phenyl ring” may mean bicyclic ring systems in which one ring of the bicyclic ring system is phenyl and the other ring is a saturated heterocycloalkyl ring. For example, the term “heterocycloalkyl fused to a phenyl ring” may refer to the group:

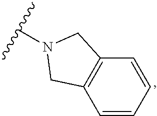 [SMILES: *N1Cc2ccccc2C1]

e.g., a 5-membered heterocycloalkyl group fused to a phenyl ring.

Aryl groups may be any aromatic carbocyclic ring system (i.e. a ring system containing 2(2n+1)π electrons). Aryl groups may have from 6 to 10 carbon atoms in the ring system. Aryl groups will typically be phenyl groups. Aryl groups may be naphthyl groups or biphenyl groups.

The term ‘heterocyclyl’ group refers to rings comprising from 1 to 4 heteroatoms independently selected from O, S and N. The rings may be heterocycloalkyl rings (including both saturated and partially saturated rings) or heteroaryl rings. The term “heterocyclyl” also encompasses groups that are tautomers of hydroxy heteroaryl groups, such pyridones, and tautomers of hydroxy heteroaryl groups that are substituted on the nitrogen, such as N-alkyl pyridones.

The term ‘heterocycloalkenyl’ refers to partially saturated rings comprising from 1 to 2 heteroatoms independently selected from O, S and N. The term “heterocycloalkenyl fused to a phenyl ring” may mean bicyclic ring systems in which one ring of the bicyclic ring system is phenyl and the other ring is a partially saturated heterocycloalkenyl ring.

The term “heteroaryl” refers to any aromatic (i.e. a ring system containing 2(2n+1)π electrons) ring system comprising from 1 to 4 heteroatoms independently selected from O, S and N (in other words from 1 to 4 of the atoms forming the ring system are selected from O, S and N). Thus, any heteroaryl groups may be independently selected from: 5 membered heteroaryl groups in which the heteroaromatic ring is substituted with 1-4 heteroatoms independently selected from O, S and N; and 6-membered heteroaryl groups in which the heteroaromatic ring is substituted with 1-3 (e.g. 1-2) nitrogen atoms. Specifically, heteroaryl groups may be independently selected from: pyrrole, furan, thiophene, pyrazole, imidazole, oxazole, isoxazole, triazole, oxadiazole, thiadiazole, tetrazole; pyridine, pyridazine, pyrimidine, pyrazine, triazine, quinoline, isoquinoline, indole, benzofuran, benzopyrazole, benzimidazole.

Compounds of the invention containing one or more asymmetric carbon atoms can exist as two or more stereoisomers. Where a compound of the invention contains a double bond such as a C═C or C═N group, geometric cis/trans (or Z/E) isomers are possible. Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of the invention, including compounds exhibiting more than one type of isomerism, and mixtures of one or more thereof. Also included are acid addition or base salts wherein the counter ion is optically active, for example, d-lactate or I-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g. 0.1% diethylamine. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Throughout the specification, a down or up wedge bond (i.e. [symbol] or [symbol] ) is used to depict absolute configuration. Where a down or up wedge bond is used at a particular position, the compound has substantially a single configuration (either R or S) at the indicated position. A down or up rectangular bond (i.e. [symbol] or [symbol] ) is used to depict relative stereochemistry between two positions. Where a down or up rectangular bond is used at a chiral centre, the compound is in the form of a mixture (typically a 1:1 mixture) of R and S configurations at the indicated position.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts.

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include ²H (also written as “D” for deuterium), ³H (also written as “T” for tritium), ¹¹C, ¹³C, ¹⁴C, ¹⁵O, ¹⁷O, ¹⁸O, ¹³N, ¹⁵N, ¹⁸F, ³⁶Cl, ¹²³I, ²⁵I, ³²P, ³⁵S and the like. The radionuclide that is used will depend on the specific application of that radio-labelled derivative. For example, for in vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

Isotopically-labelled compounds can generally be prepared by conventional techniques known to those skilled in the art or by processes analogous to those described using an appropriate isotopically-labelled reagent in place of the non-labelled reagent previously employed.

The selective replacement of hydrogen with deuterium in a compound may modulate the metabolism of the compound, the PK/PD properties of the compound and/or the toxicity of the compound. For example, deuteration may increase the half-life or reduce the clearance of the compound in vivo. Deuteration may also inhibit the formation of toxic metabolites, thereby improving safety and tolerability. It is to be understood that the invention encompasses deuterated derivatives of compounds of formula (I). As used herein, the term deuterated derivative refers to compounds of the invention where in a particular position at least one hydrogen atom is replaced by deuterium. Accordingly, in a compound of the invention one or more hydrogen atom is optionally replaced by deuterium. For example, one or more hydrogen atoms in a C₁₋₄-alkyl group may be replaced by deuterium to form a deuterated C₁₋₄-alkyl group. By way of example, if any of R^{2a}, R^{2b}, R³, R⁴, R⁵, R⁶, R⁷, R^{7a}, R^{7b}, R⁸, R^{8a}, R⁹, or R¹⁰ is methyl, the invention also encompasses —CD₃, —CHD₂ and —CH₂D. Similarly R^{2a}, R^{2b}, R³, R⁴, R⁵, R⁶, R⁷, R^{7a}, R⁸, or R^{8a} may be D.

The activity of the compounds of the present invention can be assessed by a variety of in silico, in vitro and in vivo assays. In silico analysis of a variety of compounds has been demonstrated to be predictive of ultimate in vitro and even in vivo activity.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” includes the amount of a compound that, when administered to a mammal for treating a disease, is sufficient to affect such treatment for the disease. The “therapeutically effective amount” will vary depending on the compound, the disease and its severity and the age, weight, etc., of the mammal to be treated.

A compound of the invention, or pharmaceutically acceptable salt thereof, may be used on their own but will generally be administered in the form of a pharmaceutical composition in which the compounds of the invention, or pharmaceutically acceptable salt thereof, is in association with a pharmaceutically acceptable adjuvant, diluent or carrier.

Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of the compounds of the invention, the pharmaceutical composition which is used to administer the compounds of the invention will preferably comprise from 0.05 to 99% w/w compounds of the invention, more preferably from 0.05 to 80% w/w compounds of the invention, still more preferably from 0.10 to 70% w/w compounds of the invention, and even more preferably from 0.10 to 50% w/w compounds of the invention (all percentages by weight being based on total composition).

The pharmaceutical compositions may be administered topically (e.g. to the skin) in the form, e.g., of creams, ointments, gels, lotions, solutions, suspensions; or systemically, e.g. by oral administration in the form of tablets, lozenges, hard or soft capsules, aqueous or oily suspensions, emulsions, dispersible powders or granules, syrups or elixirs; or by parenteral administration in the form of a sterile aqueous or oily solution, suspension or emulsion for injection (including intravenous, intracoronary, subcutaneous, intramyocardial, intraperitoneal, intramuscular, intravascular or infusion); by rectal administration in the form of suppositories or enemas; by inhalation for example as a finely divided powder or a liquid aerosol; or for administration by insufflation (for example as a finely divided powder).

For oral administration the compounds of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent. Thus, compositions intended for oral use may contain, for example, one or more colouring, sweetening, flavouring and/or preservative agents.

For the preparation of soft gelatine capsules, the compounds of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules. Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration the compounds of the invention may be administered as a sterile aqueous or oily solution.

The size of the dose for therapeutic or prophylactic purposes of a compound of the invention will naturally vary according to the nature and severity of the conditions, the concentration of the compound required for effectiveness in isolated cells, the concentration of the compound required for effectiveness in experimental animals, the age and sex of the animal or patient and the route of administration, according to well known principles of medicine.

Dosage levels, dose frequency, and treatment durations of compounds of the invention are expected to differ depending on the formulation and clinical indication, age, and co-morbid medical conditions of the patient.

An effective amount of a compound of the present invention for use in therapy of a condition is an amount sufficient to achieve symptomatic relief in a warm-blooded animal, particularly a human of the symptoms of the condition, to mitigate the physical manifestations of the condition, or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

For the above-mentioned compounds of the invention the dosage administered will, of course, vary with the compound employed, the mode of administration, the treatment desired and the disorder indicated. In using a compound of the invention for therapeutic or prophylactic purposes it will generally be administered so that a daily dose in the range, for example, a daily dose selected from 0.1 mg/kg to 100 mg/kg, 1 mg/kg to 75 mg/kg, 1 mg/kg to 50 mg/kg, 1 mg/kg to 20 mg/kg or 5 mg/kg to 10 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Suitably the compound of the invention is adminstered orally, for example in the form of a tablet, or capsule dosage form. The daily dose administered orally may be, for example a total daily dose selected from 1 mg to 1000 mg, 5 mg to 1000 mg, 10 mg to 750 mg or 25 mg to 500 mg. Typically, unit dosage forms will contain about 0.5 mg to 0.5 g of a compound of this invention.

The compounds of the invention may be administered along with other active compounds as part of a treatment regime. The other active compounds may be administered simultaneously with, subsequently to or previously to the administration of the compounds of the invention. It may be that the pharmaceutical formulation comprising the compounds of the invention also comprises one or more other active compounds. The other active compounds may be anticancer, anti-inflammatory, antibacterial, antiviral, antiemetic, antithrombotic or compounds that alter the metabolism.

The other active compound may be an aminoglycoside. The other active compound may be an eRF1 modulator. The other active compound may be an inhibitor or suppressor of nonsense mediated decay. The other active compound may be an SMG1 kinase inhibitor. The other active compound may be a ribosomal binder.

The compounds of the invention may be administered in combination with an aminoglycoside. In some embodiments, the compounds of the invention may allow translational read-through of PTC mutations when administered in combination with a sub-effective, sub-optimal, or sub-maximal amount of an aminoglycoside.

Aminoglycosides are compounds that have an amino-modified glycoside. Aminoglycosides are compounds that have an amino-modified glycoside. Aminoglycosides may allow translational read-through of PTC mutations. In some embodiments, when a compound of the invention is administered in combination with an aminoglycoside, the aminoglycoside may increase ribosomal read-through of mRNA transcripts carrying a PTC mutation of the cystic fibrosis CFTR channel.

Non-limiting examples of aminoglycosides include kanamycin A, amikacin, tobramycin, dibekacin, gentamicin, geneticin, sisomicin, netilmicin, neomycin B, neomycin C, paromomycin, streptomycin, plazomicin, tobramycin, ELX-02, and pharmaceutically acceptable salts thereof.

It may be that administering a compound of the invention in combination with an aminoglycoside enhances PTC read-through. It may be that administering a compound of the invention in combination with an aminoglycoside enhances PTC read-through whilst reducing the dose of aminoglycoside administered, relative to administration of the aminoglycoside alone, thereby reducing side effects, e.g. toxicity, associated with the aminoglycoside.

It may be that administering a compound of the invention in combination with an aminoglycoside increases the potency and/or beneficial effects of the aminoglycoside.

It may be that administering a compound of the invention in combination with an aminoglycoside reduces the dose of aminoglycoside required by 10%, 20%, 30%, 40%, 50%, 60%, 70%, 80%, 90%, or more, compared to administration of the aminoglycoside alone.

The compounds of the invention may be administered in combination with an eRF1 modulator, such as SRI-41315.

The compounds of the invention may be administered in combination with an inhibitor or suppressor of nonsense mediated decay, such as amlexanox, or NMD1.

The compounds of the invention may be administered in combination with an SMG1 kinase inhibitor, such as SMG1i.

The compounds of the invention may be administered in combination with an ribosomal binder, such as ZKN-013.

The compounds of the invention may be administered in combination with an antibody, for example a checkpoint inhibitor antibody. In some embodiments, the compounds of the invention may allow increased immune-cell recognition of cancer cells for use in the treatment of cancer, when administered in combination with a checkpoint inhibitor antibody. In some embodiments, the compounds of the invention may allow increased immune-cell recognition of cancer cells for use in the treatment of cancer, when administered in combination with a sub-effective, sub-optimal, or sub-maximal amount of a checkpoint inhibitor antibody.

Checkpoint inhibitor antibodies are a type of immunotherapy in oncology. Checkpoint inhibitor antibodies recognise and block different checkpoint proteins, for example, CTLA-4, PD-1 and PD-L1, which triggers immune recognition and destruction of a tumour. Immune recognition may be enhanced when a compound of the invention is administered in combination with a checkpoint inhibitor antibody, wherein the compound promotes read-through of PTCs and increases the expression of neoantigens in cancer cells.

Non-limiting examples of checkpoint inhibitor antibodies include pembrolizumab, nivolumab, cemiplimab, atezolizumab, durvalumab, avelumab, relatlimab, and ipilimumab.

It may be that administering a compound of the invention in combination with a checkpoint inhibitor antibody increases the potency and/or beneficial effects of the checkpoint inhibitor antibody.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the specification appended hereto.

The reader's attention is directed to all papers and documents which are filed concurrently with or previous to this specification in connection with this application and which are open to public inspection with this specification, and the contents of all such papers and documents are incorporated herein by reference.

The compounds of formula (I) can be made according to or analogously to General Schemes 1 to 7 and/or the following Examples.

### EXAMPLES

### Table of Abbreviations

| | |
|---|---|
| (Prep-)HPLC | (preparative-) High performance liquid |
| | chromatography |
| BINAP | (±)-2,2′-Bis(diphenylphosphino)-1,1′- |
| | binaphthalene |
| BrettPhos | 2-(Dicyclohexylphosphino)3,6-dimethoxy- |
| | 2′,4′,6′-triisopropyl-1,1′- |
| | biphenyl |
| ACN | Acetonitrile |
| DCE | Dichloroethane |
| DCM | Dichloromethane |
| DEA | Diethylamine |
| DIPEA/DIEA | N-Ethyldiisopropylamine |
| DMAc | Dimethylacetamide |
| DMEM | Dulbecco's Modified Eagle's Medium |
| DMF | Dimethylformamide |
| DMSO | Dimethylsulfoxide |
| Dppf | 1,1′-Ferrocenediyl-bis(diphenylphosphine) |
| EA/EtOAc | Ethyl acetate |
| EDA | Ethylenediamine |
| EDC/EDCI | N-Ethyl-N′-(3-dimethylaminopropyl) |
| | carbodiimide |
| EDTA | Ethylenediaminetetraacetic acid |
| EGTA | Ethylene glycol-bis(2-aminoethylether)- |
| | N,N,N′,N′-tetraacetic acid |
| EtOH | Ethanol |
| Ex | Example |
| FA | Formic acid |
| FBS | Fetal bovine serum |
| h/hr/hrs | Hours |
| HATU | 1-[Bis(dimethylamino)methylene]-1H-1,2,3- |
| | triazolo[4,5-b]pyridinium 3-oxid |
| | hexafluorophosphate |
| HBSS | Hanks' Balanced Salt solution |
| HEPES | 2-[4-(2-hydroxyethyl)piperazin-1-yl] |
| | ethanesulfonic acid |
| Hex | Hexane |
| HOBt | 1-Hydroxybenzotriazole |
| LCMS | Liquid chromatography-mass spectrometry |
| m/z | Mass/charge |
| M+ | Molecular ion |
| MeOH | Methanol |
| MES | 2-(N-morpholino)ethanesulfonic acid |
| MHz | Megahertz |
| Min/mins | Minutes |
| Ms | Mesylate |
| MTBE/MtBE | tert-Butyl methyl ether |
| NBS | N-Bromosuccinimide |
| NCS | N-Chlorosuccinimide |
| NMP | N-Methyl-2-pyrrolidone |
| NMR | Nuclear magnetic resonance |
| PBS | Phosphate Buffered Saline |
| Pd₂(dba)₃ | Tris(dibenzylideneacetone)dipalladium(0), |
| Pd-PEPPSI-IHeptCl 3- | (SP-4-1)-[1,3-Bis[2,6-bis(1-propylbutyl) |
| chloropyridine | phenyl]-4,5-dichloro-1,3-dihydro-2H- |
| | imidazol-2-ylidene]dichloro(3- |
| | chloropyridine-κN)palladium |
| Pd-PEPPSI-IPentCl 2- | (SP-4-1)-[1,3-Bis[2,6-bis(1-ethylpropyl) |
| methylpyridine (o-picoline) | phenyl]-4,5-dichloro-1,3-dihydro-2H- |
| | imidazol-2-ylidene]dichloro(2- |
| | methylpyridine)palladium |
| PE | Petroleum ether |
| PPA | Polyphosphoric acid |
| RP | Reverse phase |
| Rt/R_{T} | Retention time |
| RT/rt | Room temperature |
| STAB | Sodium triacetoxyborohydride |
| T3P | Propylphosphonic anhydride |
| TBST | Tween ® 20 Detergent |
| t-BuOK | Potassium tert-butoxide |
| TEA | Triethylamine |
| Tf | Triflate |
| TFA | Trifluoroacetic acid |
| THF | Tetrahydrofuran |
| Tris-HCl | 2-Amino-2-hydroxymethyl-propane-1,3- |
| | diol hydrochloride |
| Ts | Tosylate |
| w/w | % weight/weight |
| XantPhos | 4,5-Bis(diphenylphosphino)-9,9- |
| | dimethylxanthene |
| XPhos | 2-Dicyclohexylphosphino-2′,4′,6′- |
| | triisopropylbiphenyl |

### NMR Methods

¹H NMR spectra are recorded on Bruker AVANCE III HO 300, Bruker AVANCE NEO 400 or BrukerAVANCE IMI HO 400 spectrometers. Chemical shifts are denoted in ppm (δ) relative to residual protonated solvent as an internal standard as described in, for example, Gottlieb et al. Journal of Organic Chemistry (1997) 62 7512. The splitting pattern for NMR spectra are denoted as follows: s (singlet), br (broad), d (doublet), t (triplet), m (multiplet) or combinations thereof. Coupling constants (J) are designated in Hz and reported to one decimal place.

### Purification Methods

Liquid chromatography-mass spectra (LCMS) are recorded using the following systems and running conditions:

| | | | Initial | Middle | Gradient | Final | Gradient | | Flow | |
|---|---|---|---|---|---|---|---|---|---|---|
| | | Mobile | Water | Water | Length | Water | Length | Held | Rate | |
| Instrument | Column | Phase | (%) | (%) | (min) | (%) | (min) | (min) | (mL/min) | Condition |
| Shimadzu | EVO-C18 | Water | 100 | 50 | 2.20 | 5 | 0.20 | 0.4 | 1.50 | A |
| LCMS-2020 | 50 mm × | (0.04% | 90 | — | — | 5 | 2.30 | 0.35 | 1.50 | B |
| | 3.0 mm, | NH₃•H₂O) | 100 | 40 | 2.20 | 5 | 0.20 | 0.4 | 1.50 | C |
| | 2.6 μm | Acetonitrile | 90 | — | — | 5 | 1.45 | 0.23 | 1.50 | G |
| | | | 100 | 30 | 2.20 | 5 | 0.2 | 0.4 | 1.50 | H |
| | | | 90 | — | — | 5 | 2.30 | 0.35 | 1.50 | J |
| | | | 80 | 30 | 2.00 | 5 | 0.20 | 0.50 | 1.50 | P |
| | | | 90 | — | — | 5 | 2.10 | 0.70 | 1.50 | V |
| | | | 90 | — | — | 5 | 1.20 | 0.60 | 1.5 | X |
| | | | 90 | 30 | 2.00 | 5 | 0.10 | 0.7 | 1.5 | Z |
| | | | 90 | 40 | 2.00 | 5 | 0.10 | 0.70 | 1.5 | AE |
| | | | 90 | 30 | 2.20 | 5 | 0.20 | 0.4 | 1.5 | AH |
| | | Water | 90 | — | — | 5 | 2.00 | 0.60 | 1.50 | E |
| | | (5 mM | 90 | 30 | 2.00 | 5 | 0.20 | 0.40 | 1.50 | L |
| | | NH₄HCO₃) | 90 | — | — | 5 | 1.20 | 0.50 | 1.50 | Q |
| | | Acetonitrile | 90 | 30 | 1.80 | 5 | 0.2 | 0.60 | 1.2 | AF |
| | Poroshell | Water | 100 | 30 | 2.00 | 5 | 0.20 | 0.50 | 1.50 | D |
| | HPH-C18 | (5 mM | 100 | — | — | 5 | 2.00 | 0.60 | 1.50 | F |
| | 50 mm × | NH₄HCO₃) | 90 | 50 | 1.80 | 5 | 0.20 | 0.70 | 1.50 | AL |
| | 3.0 mm, | Acetonitrile | | | | | | | | |
| | 2.6 μm | | | | | | | | | |
| | EC-C18, | Water | 100 | 40 | 2.20 | 0 | 0.30 | 0.30 | 1.50 | I |
| | 30 | (0.05% | 100 | 30 | 2.20 | 0 | 0.20 | 0.40 | 1.50 | M |
| | mm × 3.0 | TFA)/ | 95 | 50 | 2.00 | 0 | 0.10 | 0.70 | 1.2 | AD |
| | mm, 1.9 | Acetonitrile | 95 | 30 | 2.00 | 0 | 0.10 | 0.70 | 1.5 | AJ |
| | μm | (0.05% TFA) | 95 | 40 | 2.00 | 0 | 0.10 | 0.70 | 1.5 | AK |
| | ShimNex | Water/ | 100 | — | — | 0 | 2.00 | 0.70 | 1.50 | K |
| | HE C18- | 0.05% TFA | | | | | | | | |
| | AQ 50 mm × | Acetonitrile/ | | | | | | | | |
| | 3.0 mm, | 0.05% TFA | | | | | | | | |
| | 2.6 μm | | | | | | | | | |
| | Keinetex | Water | 100 | 40 | 2.00 | 5 | 0.2 | 0.4 | 1.50 | N |
| | EVO C18, | (5 mM | | | | | | | | |
| | 50 mm × | NH₄HCO₃) | | | | | | | | |
| | 3.0 mm, | Acetonitrile | | | | | | | | |
| | 2.6 μm | | | | | | | | | |
| | Ascentis | Water | 100 | 30 | 2.00 | 0 | 0.10 | 0.50 | 1.50 | O |
| | Express | (0.05% | 95 | 40 | 1.80 | 0 | 0.20 | 0.80 | 1.50 | W |
| | 30 mm × | TFA)/CH₃CN | 95 | 30 | 2.00 | 0 | 0.10 | 0.70 | 1.5 | AA |
| | 3.0 mm, | (0.05% TFA) | 95 | — | — | 0 | 2.10 | 0.70 | 1.5 | AB |
| | 2.7 μm | | 95 | 30 | 2.0 | 0 | 0.25 | 0.35 | 1.5 | AI |
| | | | 98 | 40 | 1.80 | 0 | 0.30 | 0.70 | 1.00 | BC |
| | | Water | 95 | 50 | 2.0 | 0 | 0.10 | 0.70 | 1.50 | AU |
| | | (0.05% TFA)/ | | | | | | | | |
| | | CH₃OH | | | | | | | | |
| | HPH-C18 | Water | 90 | — | — | 5 | 2.10 | 0.70 | 1.50 | R |
| | 50 mm × | (0.4% | 90 | 30 | 2.00 | 5 | 0.10 | 0.70 | 1.5 | Y |
| | 3.0 mm, | NH₃•H₂O) | 90 | 50 | 2.00 | 5 | 0.10 | 0.70 | 1.5 | AC |
| | 2.7 μm | Acetonitrile | 98 | — | — | 5 | 2.10 | 0.7 | 1.00 | AO |
| | ChromCore | Water | 90 | 30 | 2.00 | 5 | 0.10 | 0.70 | 1.00 | S |
| | BR C18 | 0.4% | 90 | — | — | 5 | 2.00 | 0.70 | 1.5 | U |
| | 30 mm × | NH₃•H₂O) | 95 | 40 | 2.00 | 5 | 0.10 | 0.70 | 1.00 | AV |
| | 3.0 mm, | Acetonitrile | | | | | | | | |
| | 2.1 μm | | | | | | | | | |
| | HALO-C18 | Water | 95 | 30 | 1.8 | 0 | 0.20 | 0.7 | 1.5 | AG |
| | 50 mm × | (0.1% FA) | | | | | | | | |
| | 3.0 mm, | Acetonitrile | | | | | | | | |
| | 2.6 μm | (0.07% FA) | | | | | | | | |
| | NanoCHromC18 | Water/ | 95 | 30 | 1.80 | 0 | 0.20 | 0.70 | 1.00 | AM |
| | 30 mm × | 0.1% FA | | | | | | | | |
| | 3.0 mm, | Acetonitrile/ | | | | | | | | |
| | 2.1 μm | 0.07% FA | | | | | | | | |
| | EXT C18 | Water/0.04% | 90 | 30 | 2.00 | 5 | 0.1 | 0.70 | 1.00 | AN |
| | 30 mm × | NH₃•H₂O/ | 95 | 40 | 2.00 | 5 | 0.1 | 0.70 | 1.00 | AQ |
| | 2.1 mm, | Acetonitrile | 95 | / | / | 5 | 2.1 | 0.70 | 1.00 | AR |
| | 2.7 μm | | | | | | | | | |
| | Shim-pack | Water/0.04% | 90 | 30 | 2.00 | 5 | 0.1 | 0.70 | 1.00 | AP |
| | Scepter C1 | NH₃•H₂O/ | | | | | | | | |
| | 33 mm × | Acetonitrile | | | | | | | | |
| | 2.1 mm, | | | | | | | | | |
| | 3.0 μm | | | | | | | | | |
| | Titank C18 | Water/0.04% | 90 | 30 | 2.00 | 5 | 0.1 | 0.70 | 1.50 | AS |
| | 50 mm × | NH₃•H₂O/ | | | | | | | | |
| | 3.0 mm, | Acetonitrile | | | | | | | | |
| | 3.0 μm | | | | | | | | | |
| | Xtimate | Water/0.04% | 90 | 30 | 2.00 | 5 | 0.1 | 0.70 | 1.00 | AT |
| | C18 | NH₃•H₂O/ | | | | | | | | |
| | 30 mm × | Acetonitrile | | | | | | | | |
| | 2.1 mm, | | | | | | | | | |
| | 3.0 μm | | | | | | | | | |
| | Waters - | Water/0.1% | 100 | 100 | 0.50 | 0 | 1.60 | 0.70 | 0.80 | AW |
| | HSS T3 | FA | 98 | / | / | 0 | 2.10 | 0.70 | 0.80 | AX |
| | 30 mm × | Acetonitrile/ | | | | | | | | |
| | 3.0 mm, | 0.07% FA | | | | | | | | |
| | 2.5 μm | | | | | | | | | |
| | YMC- | Water/0.04% | 90 | 30 | 2.00 | 5 | 0.1 | 0.70 | 1.00 | AY |
| | Triart C18 | NH₃•H₂O/ | 90 | 5 | — | — | 2.10 | 0.70 | 1.00 | AZ |
| | 33 mm × | Acetonitrile | 95 | 40 | 2.00 | 5 | 0.10 | 0.70 | 1.00 | BA |
| | 3.0 mm, | | | | | | | | | |
| | 2.1 μm | | | | | | | | | |
| | YMC- | Water/0.1% | 100 | 98 | 2.00 | 0 | 6.00 | 2.00 | 1.00 | BB |
| | Triart C18 | FA | | | | | | | | |
| | 100 mm × | Acetonitrile/ | | | | | | | | |
| | 3.0 mm, | 0.07% FA | | | | | | | | |
| | 3.0 μm | | | | | | | | | |
| | Xtimate or | Water | 95 | 5 | 2 | 95 | 0.01 | 0.25 | 1.2 | Library A |
| | XBridge | (0.05% | | | | | | | | |
| | C18, | NH₃•H₂O) | | | | | | | | |
| | 50*3.0 | Acetonitrile | | | | | | | | |
| | mm, 2.5 | | | | | | | | | |
| | μm | | | | | | | | | |
| | Atlantis | | 95 | 20 | 3.7 | 95 | 0.01 | 0.29 | 1.2 | Library B |
| | Premier | | | | | | | | | |
| | BEH C18 | | | | | | | | | |
| | AX, | | | | | | | | | |
| | 4.6*50 | | | | | | | | | |
| | mm, 2.5 | | | | | | | | | |
| | μm | | | | | | | | | |
| | L-column3 | | 95 | 5 | 2 | 95 | 0.01 | 0.25 | 1.2 | Library C |
| | ODS, | | 95 | 20 | 3.7 | 95 | 0.01 | 0.29 | 1.2 | Library D |
| | 50*3.0 | | | | | | | | | |
| | mm, 3.0 | | | | | | | | | |
| | μm | | | | | | | | | |
| | HALO C18, | Water | 98 | 5 | 2.2 | 98 | 0.01 | 0.19 | 1.5 | Library E |
| | 50*3.0 | (0.05% TFA) | 98 | 20 | 4 | 98 | 0.01 | 0.19 | 1.5 | Library F |
| | mm, 2.7 | Acetonitrile | | | | | | | | |
| | μm | | | | | | | | | |
| | HALO AQ- | | 98 | 5 | 2.2 | 98 | 0.01 | 0.19 | 1.5 | Library G |
| | C18, | | 98 | 20 | 4 | 98 | 0.01 | 0.19 | 1.5 | Library H |
| | 50*2.1 | | | | | | | | | |
| | mm, 2 μm | | | | | | | | | |
| | ZORBAX | | 95 | 5 | 2 | 95 | 0.01 | 0.25 | 1.5 | Library I |
| | SB-Aq | | 80 | 20 | 4 | 95 | 0.01 | 0.19 | 1.5 | Library J |
| | 600 Bar, | | | | | | | | | |
| | 0*4.6 mm, | | | | | | | | | |
| | 1.8 μm | | | | | | | | | |
| | Kinetex | Water | 98 | 5 | 2.2 | 98 | 0.01 | 0.29 | 1.2 | Library K |
| | EVO C18, | (0.05% | | | | | | | | |
| | 50*3.0 | NH₃•H₂O) | | | | | | | | |
| | mm, | Acetonitrile | | | | | | | | |
| | 2.6 um | | | | | | | | | |

Purification by preparative HPLC (prep-HPLC) employs the following instruments and conditions:

| | | | Initial | Final | | Gradient | |
|---|---|---|---|---|---|---|---|
| | | | Water | Water | Flow Rate | Length | |
| Instrument | Column | Mobile Phase | (%) | (%) | (mL/min) | (min) | Condition |
| Biotage | C18 | MeCN in Water (0.1% FA) | 80 | 70 | 60 | 10 | A |
| Isolera | | | 100 | 0 | 60 | 20 | B |
| Prime | | | 55 | 45 | 40 | 10 | S |
| | | Water (0.1% FA)/MeOH | 50 | 5 | 60 | 10 | AC |
| | | Water (0.05% TFA)/MeOH | 100 | 0 | 60 | 20 | BB |
| | | Water (0.1% | 100 | 20 | 60 | 30 | C |
| | | NH₃•H₂O)/MeOH (5% | 35 | 20 | 60 | 10 | R |
| | | NH₃•H₂O) | 45 | 35 | 60 | 10 | AM |
| | | | 100 | 0 | 60 | 20 | AV |
| | | Water (0.05% | 100 | 0 | 60 | 30 | L |
| | | NH₃•H₂O)/MeOH | | | | | |
| | | Water (0.1% | 80 | 70 | 60 | 10 | AD |
| | | NH₃•H₂O)/MeCN | 30 | 10 | 60 | 10 | AH |
| | | | 100 | 0 | 60 | 20 | AW |
| | | Water (10 mmol/L | 85 | 75 | 60 | 20 | P |
| | | NH₄HCO₃)/CH₃CN | | | | | |
| | | Water/MeOH | 100 | 0 | 60 | 30 | BE |
| Waters | XBridge Shield | Water | 86 | 73 | 60 | 10 | D |
| 2545 Binary | RP18 OBD | (10 mM NH₃•H₂O) | 95 | 65 | 100 | 35 | U |
| Gradient | Column 30 × | Acetonitrile | 50 | 32 | 60 | 10 | AB |
| Module | 150 mm, 5 μm | | 91 | 76 | 60 | 10 | AG |
| with Waters | | Water | 57 | 37 | 60 | 10 | AL |
| 2489 | | (10 mM NH4HCO3 + 0.1% | 74 | 54 | 60 | 10 | AO |
| UV/visible | | NH3•H2O) | 95 | 80 | 60 | 10 | AR |
| detector | | Acetonitrile | 80 | 62 | 60 | 10 | AY |
| | | Water | 86 | 66 | 60 | 12 | AU |
| | | (10 mM NH4HCO3 + 0.1% | 66 | 46 | 60 | 10 | BA |
| | | NH3•H2O) | | | | | |
| | | MeOH | | | | | |
| | YMC-Actus | Water (0.05% | 100 | 60 | 40 | 5 | E |
| | Triart C18 ExRS, | NH₃•H₂O)/MeOH | | | | | |
| | 30 × 150 mm, 5 | Water (0.05% | 94 | 77 | 60 | 10 | AI |
| | μm | NH₃•H₂O)/CH3CN | | | | | |
| | | MeOH in Water (10 mmol/L | 73 | 56 | 60 | 10 | BD |
| | | NH₄HCO₃) | | | | | |
| | XBridge Prep | Water (0.1% FA)/ACN | 90 | 70 | 60 | 10 | AT |
| | Phenyl OBD | Water | 98 | 88 | 60 | 10 | AZ |
| | Column 19 × | (0.05% TFA) | | | | | |
| | 250 mm, 5 μm | Acetonitrile | | | | | |
| | | Water (10 mmol/L | 70 | 54 | 40 | 10 | F |
| | | NH₄HCO₃ + 0.1% | 72 | 52 | 60 | 10 | K |
| | | NH₃•H₂O)/ACN | 74 | 60 | 60 | 10 | M |
| | | | 95 | 78 | 60 | 10 | N |
| | | | 82 | 67 | 60 | 10 | W |
| | | | 100 | 88 | 60 | 10 | X |
| | | | 89 | 74 | 60 | 10 | Y |
| | | | 55 | 25 | 60 | 10 | Z |
| | | Water (0.1% | 56 | 36 | 60 | 10 | AE |
| | | NH₃•H₂O)/MeOH | | | | | |
| | | Water (0.1% | 97 | 79 | 60 | 10 | AJ |
| | | NH₃•H₂O)/CH₃CN | | | | | |
| | | Water (10 mmol/L | 80 | 64 | 60 | 10 | G |
| | | NH₄HCO₃ + 0.1% | 65 | 49 | 60 | 10 | O |
| | | NH₃•H₂O)/ACN (1% 2 mM | | | | | |
| | | NH₃—MeOH) | | | | | |
| | | Water (10 mmol/L | 73 | 57 | 60 | 10 | J |
| | | NH₄HCO₃ + 0.1% | | | | | |
| | | NH₃•H₂O)/ACN (1% 2 mM | | | | | |
| | | NH₃—MeOH) | | | | | |
| | | Water (10 mmol/L | 70 | 59 | 60 | 10 | AF |
| | | NH₄HCO₃ + 0.1% | 72 | 53 | 60 | 10 | AN |
| | | NH₃•H₂O)/MeOH | 81 | 56 | 60 | 10 | AS |
| | | | 79 | 64 | 60 | 10 | AX |
| | YMC-Actus | Water | 97 | 80 | 60 | 10 | H |
| | Triart C18 ExRS, | (10 mM NH₄HCO₃) | 86 | 69 | 60 | 10 | T |
| | 30 × 150 mm, 5 | Acetonitrile | | | | | |
| | μm | Water (10 mmol/L | 85 | 68 | 60 | 10 | I |
| | | NH₄HCO₃ + 0.05% | 65 | 50 | 60 | 8 | Q |
| | | NH₃•H₂O)/ACN | 98 | 84 | 60 | 10 | V |
| | | Water (10 mmol/L | 75 | 55 | 60 | 10 | AK |
| | | NH₄HCO₃ + 0.05% | 95 | 75 | 60 | 10 | AP |
| | | NH₃•H₂O)/MeOH | 64 | 49 | 60 | 10 | AQ |
| | | | 49 | 32 | 60 | 15 | BC |
| | XB C-18, | Water(10 mmol/L | 95 | 5 | 100 | 30 | AA |
| | 50*250 mm, 10 | NH4HCO3 + 0.05% | | | | | |
| | μm | NH3•H2O)•MeOH | | | | | |
| Agela MP- | WelFlash C18, | Water (10 mmol/L | 85 | 25 | 80 | 10 | Library |
| Flash200 | 120 g, 20-40 μm | NH₄HCO₃ + 0.05% | | | | | |
| | | NH₃•H2O)/ACN | | | | | |

Separation by SF0 employs the following instruments and conditions:

| | | | | Initial | Final | | Gradient | |
|---|---|---|---|---|---|---|---|---|
| | | Mobile | Temperature/ | B | B | Flow Rate | Length | |
| Instrument | Column | Phase | Pressure | (%) | (%) | (mL/min) | (min) | Condition |
| Waters Prep | NB_CHIRALPAK | A: CO₂ | 35° C./120 bar | 20 | 20 | 100 | 10 | A |
| SFC 150 | AD, 3 × 25 cm, 5 | B: MeOH | | | | | | |
| | μm | | | | | | | |
| HP-Flash | CHIRALPAK IC | A: 3:1 | 25° C./50 Bar | 20 | 20 | 25 | 10 | Library A |
| | 2*25 5 μm | Hex:DCM | | | | | | |
| | | B: EtOH | | | | | | |
| | | (0.2% EDA) | | | | | | |
| Gilson | CHIRAL ART | A: 3:1 | 25° C./45 Bar | 20 | 20 | 35 | 10 | Library B |
| | Cellulose-SJ 3*25 | Hex:DCM | | | | | | |
| | 5 μm | B: EtOH | | | | | | |
| | | (0.2% EDA) | | | | | | |
| | CHIRALPAK IC | A: 3:1 | 25° C./50 Bar | 50 | 50 | 25 | 10 | Library C |
| | 3*25 5 μm | Hex:DCM | | | | | | |
| | | B: EtOH | | | | | | |
| | | (0.2% EDA) | | | | | | |
| | CHIRALPAK IE | A: MeOH | 25° C./45 Bar | 50 | 50 | 35 | 10 | Library D |
| | 3*25 5 μm | B: EtOH | | | | | | |
| | | (0.2% EDA) | | | | | | |
| Waters Prep | CHIRALPAK IG | A: CO₂ | 35° C./65 bar | 50 | 50 | 80 | 10 | Library E |
| SFC-150 | 3*25 5 μm | B: 2:1 | | | | | | |
| | | MeOH:DCM | | | | | | |
| | | (0.2% 2 mmol | | | | | | |
| | | NH₃—MeOH) | | | | | | |

Separation by Chiral-HPLC employs the following instruments and conditions:

| | | | Initial | Final | | Gradient | |
|---|---|---|---|---|---|---|---|
| | | Mobile | B | B | Flow Rate | Length | |
| Instrument | Column | Phase | (%) | (%) | (mL/min) | (min) | Condition |
| Gilson | Lux 5 u Cellulose-2, | A: Hex | 20 | 20 | 40 | 40 | A |
| GX-281 | 30*250 mm, 5.0 | (0.1% DEA) | | | | | |
| | um | B: EtOH | | | | | |
| | CHIRAL ART | A: Hex | 15 | 15 | 40 | 15 | B |
| | Cellulose-SC, | (10 mM | | | | | |
| | 3*25 cm, 5 μm | NH₃—MeOH) | | | | | |
| | | B: EtOH | | | | | |
| | CHIRALPAK IG | A: Hex | 50 | 50 | 40 | 15 | C |
| | 3*25 cm, 5 μm | (10 mM | | | | | |
| | | NH₃—MeOH) | | | | | |
| | | B: EtOH | | | | | |

### Process for Preparation:

Certain compounds of the invention may be synthesised according to the general methods disclosed herein. Certain compounds of the invention may be synthesised according to general schemes i to 7. Certain compounds of the invention may be synthesised according to or analogously to the syntheses provided in the examples.

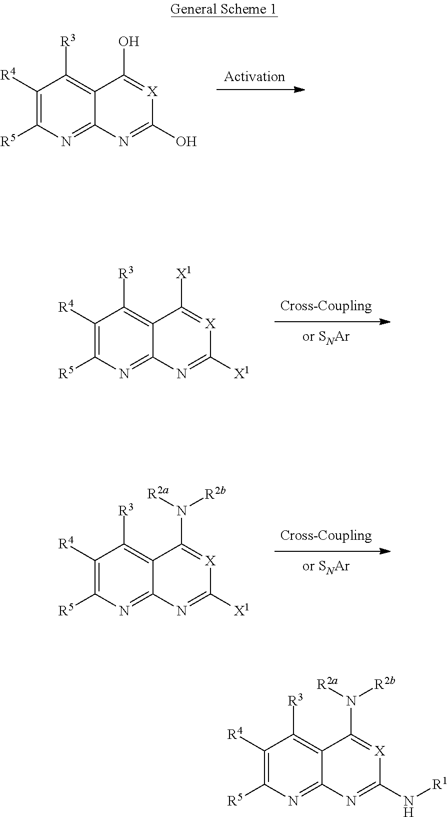

General Scheme 1 illustrates a route to compounds of Formula (I) and is described in the following examples.

Appropriately functionalised starting materials can be activated into a suitable leaving group X¹ (where X¹═Cl, Br, OMs, OTs, OTO) by use of conditions well-known to those skilled in the art such as, for example, POCl₃, POBr₃, triflic anhydride, mesyl chloride or tosyl chloride in the presence of a suitable base if necessary (for example TEA, DIPEA), in a suitable solvent (for example DCM, THF) if necessary, with heating if required.

Sequential displacement of X¹ can be achieved by reacting an amine in a cross-coupling reaction (where X¹ is, for example, Cl, Br, OTf) in the presence of a suitable catalyst (for example palladium(II) acetate, Brettphos Pd G3, Pd₂(dba)₃), with a ligand if necessary (for example, Brettphos, XantPhos) with a suitable base (for example Na₂CO₃, Cs₂CO₃) in a suitable solvent (for example 1,4-dioxane, toluene, THF) with heating (conventional or by microwave irradiation) if required.

Alternatively, sequential displacement of X¹ can be achieved by reacting an amine in a substitution reaction (where X¹ is, for example, Cl, OMs, OTs) conducted in the presence of a suitable base (for example TEA, KF, CsF, potassium tert-butoxide, Na₂CO₃, Cs₂CO₃) in a suitable solvent (for example 1,4-dioxane, DMSO, THF, NMP, or neat conditions) with heating (conventional or by microwave irradiation) if required.

It will be well understood by those skilled in the art that either two coupling reactions, two substitution reactions or a mixture of coupling and substitution reactions may be required for the sequential, regioselective displacement of both X¹ groups.

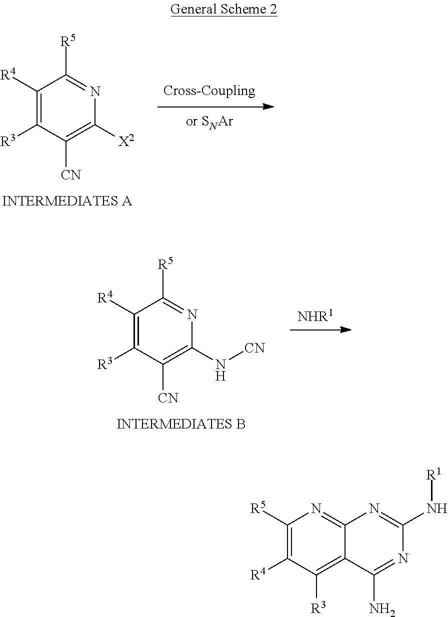

General Scheme 2 illustrates a route to compounds of Formula (III) and is described in the following examples.

A cross-coupling reaction of suitably functionalised Intermediates A (where X² is, for example, Cl, Br, I, OSO₂CF₃) with cyanamide can be conducted in the presence of a suitable catalyst (for example palladium(II) acetate, Brettphos Pd G3, Pd₂(dba)₃), with a ligand if necessary (for example, Brettphos, XantPhos) with a suitable base (for example Na₂CO₃, Cs₂CO₃) in a suitable solvent (for example 1,4-dioxane, toluene, THF) with heating (conventional or by microwave irradiation) if required.

Alternatively, a nucleophilic aromatic substitution (S_{N}Ar) reaction of suitably functionalised Intermediates A (where X² is, for example, F, Cl, OMs, OTs) with cyanamide can be conducted in the presence of a suitable base (for example TEA, KF, CsF, potassium tert-butoxide, Na₂CO₃, Cs₂CO₃) in a suitable solvent (for example 1,4-dioxane, DMSO, THF, NMP, or neat conditions) with heating (conventional or by microwave irradiation) if required.

The resultant cyano-cyanamide product can be cyclised by reaction with an appropriate nucleophile (for example an amine or hydroxylamine), using an appropriate base if necessary (for example triethylamine, DIPEA, potassium carbonate), in an appropriate solvent (for example DCM, DMSO, DMF, NMP, THF, 1,4-dioxane, ethanol, methanol) with heating (conventional or by microwave irradiation) if required.

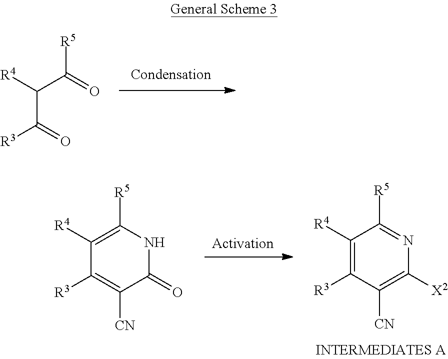

Intermediates A may be commercially available; synthesised using methods similar to those previously described in, for example, WO2005097750 A1, CN109232412 A or WO2013089573 A1; or according to General Scheme 3 and as described in the following examples.

A condensation of suitably functionalised 1,3-diadehydes or ketones with 2-cyanoacetamide may be carried out in the presence of a suitable base (for example, piperidine, sodium hydride) in a suitable solvent (for example, ethanol, DMSO) with heating if required.

The resultant pyrimidone intermediates can be activated into a suitable leaving group X² (where X² is, for example, F, Cl, Br, I, OMs, OTs, OTf) by use of conditions well-known to those skilled in the art such as, for example, POCl₃, POBr₃, triflic anhydride, mesyl chloride or tosyl chloride in the presence of a suitable base if necessary (for example TEA, DIPEA), in a suitable solvent (for example DCM, THF) if necessary, with heating if required.

It will be appreciated by those skilled in the art that the condensation step may result in mixture of isomers in varying ratios which may be separated using well-known techniques (for example, crystallisation, normal, reverse and super-critical fluid chromatography). Separation may be conducted after the condensation step, or after activation of the crude mixture.

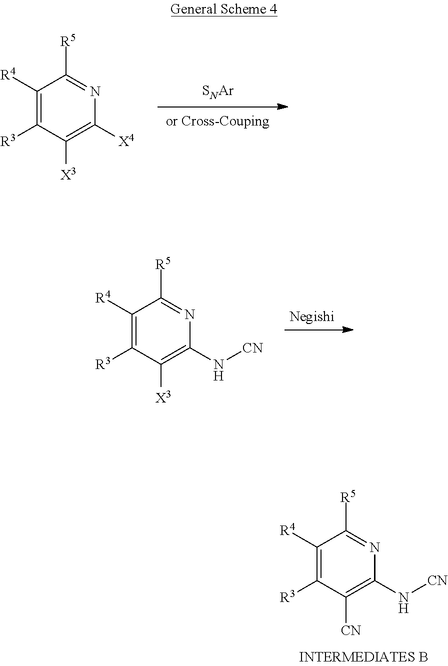

Alternatively, Intermediates B may be synthesised according to General Scheme 4.

A nucleophilic aromatic substitution (S_{N}Ar) reaction of suitably functionalised starting material (where X⁴ is, for example, F, Cl and X³ is, for example, Br, I) with cyanamide can be conducted in the presence of a suitable base (for example TEA, KF, CsF, potassium tert-butoxide, Na₂CO₃, Cs₂CO₃) in a suitable solvent (for example 1,4-dioxane, DMSO, THF, NMP, or neat conditions) with heating (conventional or by microwave irradiation) if required.

A cross-coupling reaction of suitably functionalised starting material (where X⁴ is, for example, I, and X³ is, for example, Cl, Br) with cyanamide can be conducted in the presence of a suitable catalyst (for example palladium(II) acetate, Brettphos Pd G3, Pd₂(dba)₃), with a ligand if necessary (for example, Brettphos, XantPhos) with a suitable base (for example Na₂CO₃, Cs₂CO₃) in a suitable solvent (for example 1,4-dioxane, toluene, THF) with heating (conventional or by microwave irradiation) if required.

The cyano group can be introduced into the resultant intermediates by way of a Negishi coupling using, for example, zinc cyanide in the presence of a suitable palladium catalyst (for example Pd₂dba₃) with a suitable ligand (for example triphenylphosphine or BINAP) and in a suitable solvent (for example 1,4-dioxane) with heating if necessary.

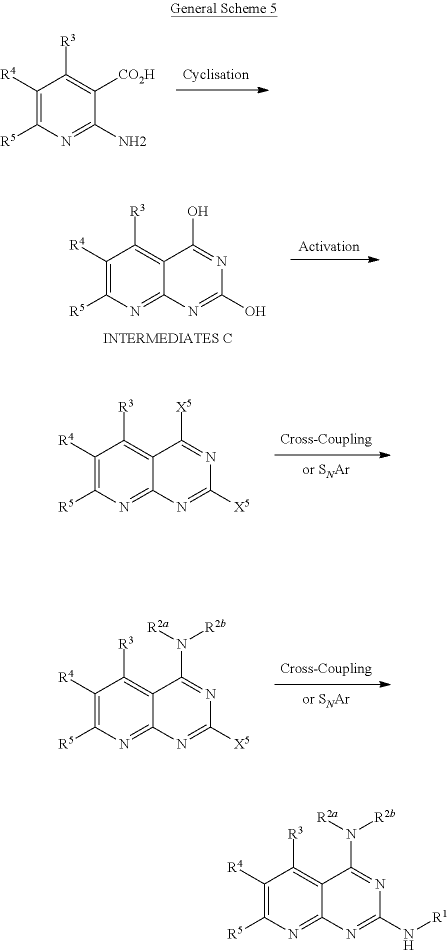

General Scheme 5 illustrates a route to compounds of Formula (II) and is described in the following examples.

Appropriately substituted starting materials can be cyclised to furnish Intermediates C using, for example, potassium cyanate with ammonium chloride in water with heating if necessary, as described in, for example, WO2006090167 A2 or Bioorganic & Medicinal Chemistry Letters (2009), 19(20), 5950-5953.

Alternatively, appropriately substituted starting materials can be cyclised to furnish Intermediates C using, for example, urea (neat or in a suitable solvent such as water, for example), in the presence of sodium hydroxide if necessary, with heating if necessary, as described in, for example, Tetrahedron (2012), 68(43), 8908-8915; Journal of Medicinal Chemistry (2016), 59(4), 1370-1387; US20070281949 A1 or WO2005049033 A1.

The resultant intermediates can be activated into a suitable leaving group X⁵ (where X⁵═Cl, Br, OMs, OTs, OTf) by use of conditions well-known to those skilled in the art such as, for example, POCl₃, POBr₃, triflic anhydride, mesyl chloride or tosyl chloride in the presence of a suitable base if necessary (for example TEA, DIPEA), in a suitable solvent (for example DCM, THF) if necessary, with heating if required.

Sequential displacement of X⁵ can be achieved by reacting an amine in a cross-coupling reaction (where X⁵ is, for example, Cl, Br, OTf) in the presence of a suitable catalyst (for example palladium(II) acetate, Brettphos Pd G3, Pd₂(dba)₃), with a ligand if necessary (for example, Brettphos, XantPhos) with a suitable base (for example Na₂CO₃, Cs₂CO₃) in a suitable solvent (for example 1,4-dioxane, toluene, THF) with heating (conventional or by microwave irradiation) if required.

Alternatively, sequential displacement of X⁵ can be achieved by reacting an amine in a substitution reaction (where X⁵ is, for example, Cl, OMs, OTs) conducted in the presence of a suitable base (for example TEA, KF, CsF, potassium tert-butoxide, Na₂CO₃, Cs₂CO₃) in a suitable solvent (for example 1,4-dioxane, DMSO, THF, NMP, or neat conditions) with heating (conventional or by microwave irradiation) if required.

It will be well understood by those skilled in the art that either two coupling reactions, two substitution reactions or a mixture of coupling and substitution reactions may be required for the sequential, regioselective displacement of both X⁵ groups.

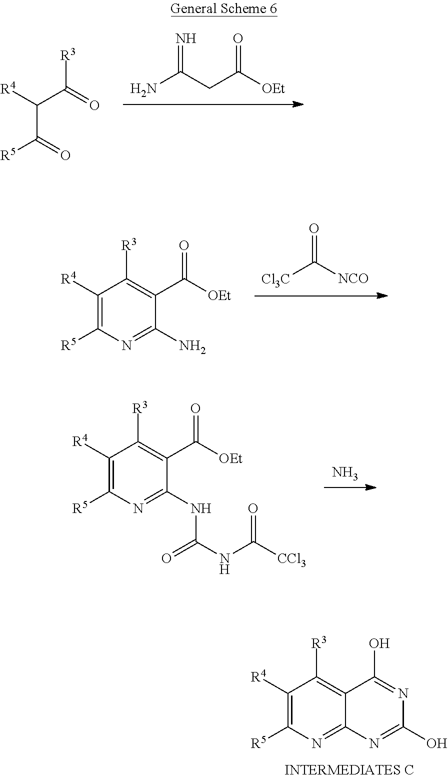

Alternatively, Intermediates C may be synthesised according to General Scheme 6 and as described in the following Examples.

An appropriate dicarbonyl starting material can be condensed with a suitable amidine ester (for example ethyl 3-amino-3-iminopropanoate as a free base or salt, for example hydrochloride) in the presence of a suitable base (for example piperidine, DIPEA, TEA, potassium tert-butoxide, Na₂CO₃, sodium hydroxide, pyridine) in a suitable solvent (for example methanol, ethanol, isopropanol, dichloroethane, acetonitrile, 1,4-dioxane, tetrahydrofuran, toluene, DMF, DMSO) with heating (conventional or by microwave irradiation) if required.

It will be appreciated by those skilled in the art that the condensation step may result in mixture of isomers in varying ratios which may be separated using well-known techniques (for example, crystallisation, normal, reverse and super-critical fluid chromatography).

The resultant amino group can be activated with a suitable isocyanate (for example 2,2,2-trichloroacetyl isocyanate) in a suitable solvent (for example dichloroethane, 1,4-dioxane, tetrahydrofuran, toluene) with heating (conventional or by microwave irradiation) if required.

The resultant compound can be cyclised in the presence of ammonia in a suitable solvent (for example methanol, ethanol, isopropanol) with heating (conventional or by microwave irradiation) if required, under pressure if required.

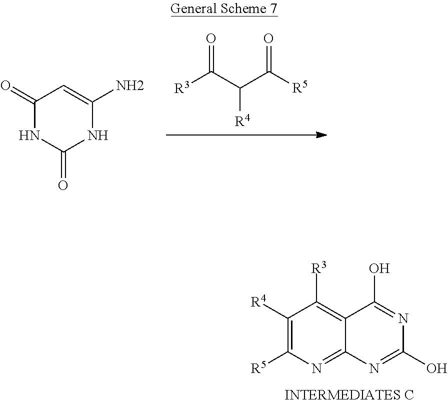

Alternatively, Intermediates C may be synthesised according to General Scheme 7 and as described in the following Examples.

An appropriate starting material (for example, 6-aminouracil) can be condensed with a suitable diketone (for example acetylacetone) in the presence of a suitable acid (for example polyphosphoric acid) with heating (conventional or by microwave irradiation) if required.

It will be appreciated by those skilled in the art that the condensation step may result in mixture of isomers in varying ratios which may be separated using well-known techniques (for example, crystallisation, normal, reverse and super-critical fluid chromatography).

### Example 1: N²-(1-methylpiperidin-4-yl)pyrido[2,3-d]pyrimidine-2,4-diamine

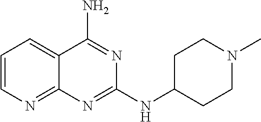 [SMILES: CN1CCC(Nc2nc(N)c3cccnc3n2)CC1]

### Route A

### Preparation 1—2-(cyanoamino) pyridine-3-carbonitrile

To a solution of 2-chloropyridine-3-carbonitrile (1.00 g, 7.22 mmol) in NMP (10.0 mL) at room temperature was added cyanamide, monosodium salt (690 mg, 10.8 mmol). The mixture was stirred at 60° C. overnight. The mixture was directly purified by silica gel column chromatography (eluted with EtOAc/MeOH (from 0 to 100%)) to afford the title compound as a yellow solid (1.0 g, crude). LCMS: m/z=145 [M+H]⁺.

### Preparation 2—N²-(1-methylpiperidin-4-yl)pyrido[2,3-d]pyrimidine-2,4-diamine

To a solution of 2-(cyanoamino) pyridine-3-carbonitrile (600 mg, 4.16 mmol) in 1,4-dioxane (10.0 mL) at room temperature was added 1-methylpiperidin-4-amine (570 mg, 5.00 mmol). The mixture was stirred at 100° C. overnight. The resulting mixture was concentrated under reduced pressure. The crude product was purified by Prep-HPLC^{G} to afford the title compound as a yellow solid (30.7 mg, 2.8%). ¹H NMR (400 MHz, DMSO-d⁶) δ 8.62-8.56 (m, 1H), 8.36-8.30 (m, 1H), 7.39 (br, 2H), 6.98 (s, 1H), 6.56 (br, 1H), 3.87-3.73 (m, 1H), 2.79-2.70 (m, 2H), 2.16 (s, 3H), 1.99-1.89 (m, 2H), 1.86-1.78 (m, 2H), 1.58-1.44 (m, 2H). LCMS^{B}: m/z=259 [M+H]⁺.

### Route B

### Preparation 3—2-chloropyrido[2,3-d]pyrimidin-4-amine

Into a 40 mL vial were added 2,4-dichloropyrido[2,3-d]pyrimidine (500 mg, 2.50 mmol) and ammonium hydroxide (5.0 mL) at room temperature. The resulting mixture was stirred overnight at 80° C. The resulting mixture was concentrated under reduced pressure. The residue was purified by trituration with MtBE (8.0 mL). This resulted in title compound as a light-yellow solid (430 mg, crude). LCMS m/z=181 [M+H⁺]

### Preparation 4—N²-(1-methylpiperidin-4-yl)pyrido[2,3-d]pyrimidine-2,4-diamine

To a stirred solution of 2-chloropyrido[2,3-d]pyrimidin-4-amine (200 mg, 1.11 mmol) in DMSO (5.0 mL) was added Cs₂CO₃ (721 mg, 2.21 mmol) and 1-methylpiperidin-4-amine (252 mg, 2.21 mmol) in portions at room temperature. The resulting mixture was stirred at 100° C. for 12 h. The mixture was allowed to cool down to room temperature. The resulting mixture was filtered through a short pad of Celite. The combined filtrate was purified by Prep-HPLC^{AS} to afford the title compound as a light yellow solid (5 mg, 1.7%). ¹H NMR (400 MHz, DMSO-d₆) δ 8.59 (d, J=3.6 Hz, 1H), 8.36-8.33 (m, 1H), 7.38 (br., 2H), 6.97 (s, 1H), 6.66 (br s, 1H), 3.91-3.64 (m, 1H), 2.75-2.70 (m, 2H), 2.16 (s, 3H), 2.00-1.89 (m, 2H), 1.81-1.80 (m, 2H), 1.51-1.50 (m, 2H). LCMS^{H} m/z=259 [M+H⁺]

### Example 2: 5-methyl-N²-(1-methylpiperidin-4-yl)pyrido[2,3-d]pyrimidine-2,4-diamine

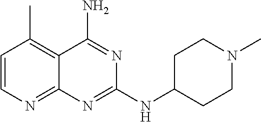 [SMILES: Cc1ccnc2nc(NC3CCN(C)CC3)nc(N)c12]

### Preparation 5—N-(3-cyano-4-methylpyridin-2-yl) cyanamide

To a stirred solution of 2-chloro-4-methylpyridine-3-carbonitrile (1.00 g, 6.55 mmol) in NMP (20.0 mL) at room temperature was added cyanamide, monosodium salt (840 mg, 13.1 mmol). The resulting mixture was stirred at 60° C. for 1 h. The mixture was acidified to pH=6 with conc. HCl. The resulting mixture was filtered, the filter cake was washed with water (3×3.00 mL). The filtrate was concentrated under reduced pressure. The residue was purified by Prep-HPLC^{B} to afford the title compound as a white solid (560 mg, 54.0%). LCMS: m/z=159 [M+H]⁺.

### Preparation 6—5-methyl-N²-(1-methylpiperidin-4-yl)pyrido[2,3-d]pyrimidine-2,4-diamine

A solution of 2-(cyanoamino)-4-methylpyridine-3-carbonitrile (200 mg, 1.27 mmol) and 1-methylpiperidin-4-amine (0.19 mL, 1.52 mmol) in EtOH (4.00 mL) was stirred at 80° C. for 6 h. The resulting mixture was concentrated under reduced pressure. The residue was purified by Prep-HPLC^{C}. The residue was purified by trituration with MTBE (10.0 mL) to afford the title compound as white solid (100.4 mg, 29.2%). ¹H NMR (400 MHz, DMSO-d⁶) δ 8.38 (s, 1H), 7.00-6.33 (m, 4H), 3.85-3.72 (m, 1H), 2.74- (d, J=11.1 Hz, 2H), 2.68 (s, 3H), 2.16 (s, 3H), 2.00-1.86 (m, 2H), 1.89-1.81 (m, 2H), 1.56-1.46 (m 2H). LCMS^{A}: m/z=273 [M+H]⁺.

### Example 3: 7-methyl-N²-(1-methylpiperidin-4-yl)pyrido[2,3-d]pyrimidine-2,4-diamine

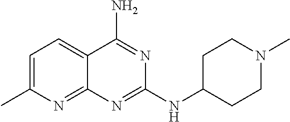

### Preparation 7N-(3-cyano-6-methylpyridin-2-yl)cyanamide

To a mixture of 2-chloro-6-methylpyridine-3-carbonitrile (1.00 g, 6.55 mmol) in NMP (10.0 mL) was added cyanamide, monosodium salt (839 mg, 13.1 mmol). The mixture was stirred at 60° C. overnight. The resulting mixture was diluted with water (30.0 mL) and acidified to pH 6.5 by the addition of aq. HCl (1.0 M). The product was precipitated, filtered and dried to afford the title compound as a yellow solid (555.4 mg, 53.6%). LCMS: m/z=159 [M+H]⁺.

### Preparation 2: 7-methyl-N²-(1-methylpiperidin-4-yl)pyrido[2,3-d]pyrimidine-2,4-diamine

To a mixture of 2-(cyanoamino)-6-methylpyridine-3-carbonitrile (300 mg, 1.90 mmol, 1.00 equiv) in EtOH (10.0 mL) was added 1-methylpiperidin-4-amine (0.28 mL, 2.28 mmol, 1.20 equiv). The mixture was stirred at 80° C. overnight. The resulting mixture was concentrated under reduced pressure. The residue was purified by Prep-HPLC^{E}. The product was further purified by trituration with MTBE (10 mL) to afford the title compound as a light-yellow solid (81 mg, 15.9%). ¹H NMR (300 MHz, DMSO-d⁶) δ 8.22 (d, J=8.1 Hz, 1H), 7.29 (br, 2H), 6.88 (d, J=8.1 Hz, 1H), 6.50 (br, 1H), 3.87-3.64 (m, 1H), 2.82-2.62 (m, 2H), 2.45 (s, 3H), 2.16 (s, 3H), 2.00-1.88 (m, 2H), 1.81-1.73 (m, 2H), 1.53-1.49 (m, 2H). LCMS^{A}: m/z=273 [M+H]⁺.

### Example 4: 6-methyl-N²-(1-methylpiperidin-4-yl)pyrido[2,3-d]pyrimidine-2,4-diamine

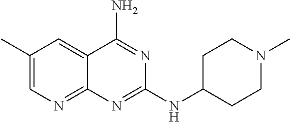

### Preparation 8—N-(3-cyano-5-methylpyridin-2-yl) cyanamide

To a solution of 2-chloro-5-methylpyridine-3-carbonitrile (400 mg, 2.62 mmol) in DMSO (4.0 mL) at room temperature was added cyanamide, monosodium salt (252 mg, 3.93 mmol). The mixture was stirred at 60° C. for 2 h. The reaction was quenched with water. The mixture was acidified to pH 6 with aq. HCl (1M). The precipitated solids were collected by filtration and washed with EtOAc (3×30 mL). The crude product was further purified by trituration with MTBE (5.0 mL). The precipitated solids were dried under vacuum to afford the title compound as a grey solid (390 mg, 94.1%). LCMS: m/z=159 [M+H]⁺.

### Preparation 2: 6-methyl-N²-(1-methylpiperidin-4-yl)pyrido[2,3-d]pyrimidine-2,4-diamine

To a stirred solution of 2-(cyanoamino)-5-methylpyridine-3-carbonitrile (100 mg, 0.632 mmol, 1.00 equiv) in EtOH (2.0 mL) at room temperature was added 1-methylpiperidin-4-amine (86.6 mg, 0.758 mmol, 1.20 equiv). The mixture was stirred at 80° C. overnight. The resulting mixture was concentrated under reduced pressure. The crude product was purified by Prep-HPLC^{E} to afford the title compound as a white solid (29.6 mg, 16.5%). ¹H NMR (400 MHz, MeOD) δ 8.49 (s, 1H), 8.13 (s, 1H), 4.02-3.92 (m, 1H), 2.86 (d, J=11.7 Hz, 2H), 2.38 (s, 3H), 2.30 (s, 3H), 2.27-2.17 (m, 2H), 2.09-2.01 (m, 2H), 1.66-1.53 (m, 2H). LCMS^{B}: m/z=273 [M+H]⁺.

### Example 5: 5,7-dimethyl-N²-(1-methylpiperidin-4-yl)pyrido[2,3-d]pyrimidine-2,4-diamine

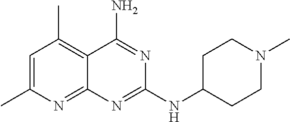

### Route A

### Preparation 9—2-(cyanoamino)-4,6-dimethylpyridine-3-carbonitrile

To a stirred mixture of 2-chloro-4,6-dimethylpyridine-3-carbonitrile (30.0 g, 180 mmol) in DMSO (300 mL) at room temperature was added cyanamide, monosodium salt (22.7 g, 360 mmol). The resulting mixture was stirred at 60° C. for 2 h. The reaction was quenched by the addition of water (600 mL). The residue was acidified to pH 6 with aq. HCl (1 M). The precipitated solids were collected by filtration and washed with MTBE (3×100 mL). The filter cake was dried under vacuum to afford the title compound as a brown solid (25.0 g, crude). LCMS: m/z=173 [M+H]⁺.

### Preparation 10—5,7-dimethyl-N²-(1-methylpiperidin-4-yl)pyrido[2,3-d]pyrimidine-2,4-diamine

To a stirred mixture of 2-(cyanoamino)-4,6-dimethylpyridine-3-carbonitrile (20.0 g, 116 mmol) in EtOH (400 mL) at room temperature was added 1-methylpiperidin-4-amine (15.9 g, 139 mmol) dropwise. The resulting mixture was stirred at 80° C. overnight. The resulting mixture was concentrated under reduced pressure. The residue was purified by Prep-HPLC^{A} and the product was triturated with MTBE (50 mL) to afford the title compound as a brown solid (10.6 g, 31.6%). ¹H NMR (300 MHz, DMSO-d⁶) δ 6.67 (s, 3H), 6.38 (s, 1H), 3.84-3.69 (m, 1H), 2.74 (d, J=11.6 Hz, 2H), 2.63 (s, 3H), 2.37 (s, 3H), 2.16 (s, 3H), 2.02-1.88 (m, 2H), 1.81-1.78 (m, 2H), 1.56-1.48 (m, 2H). LCMS^{A}: m/z=287 [M+H]⁺.

### Route B

### Preparation 11—5,7-dimethylpyrido[2,3-d]pyrimidine-2,4-diol

To a stirred mixture of 6-aminouracil (50.0 g, 393 mmol) in acetylacetone (200 mL) at room temperature was added PPA (90.5 g, 786 mmol). The resulting mixture was stirred at 100° C. for 1 h. The mixture was allowed to cool down to room temperature. The reaction was quenched with NaHCO₃(aq) at 0° C. The precipitated solids were collected by filtration and washed with water (2×300 mL). The solid was further purified by trituration with MtBE (300 mL). This resulted in title compound as an off-white solid (38.0 g, crude). LCMS m/z=192 [M+H]⁺

### Preparation 12—2,4-dichloro-5,7-dimethylpyrido[2,3-d]pyrimidine

To a stirred mixture of 5,7-dimethylpyrido[2,3-d]pyrimidine-2,4-diol (15.5 g, 81.1 mmol) in POCl₃ (155 mL) at 0° C. was added N,N-diethylaniline (60.5 g, 405 mmol) dropwise. The resulting mixture was stirred at 60° C. for 3 h. The resulting mixture was allowed to cool down to room temperature and concentrated under reduced pressure. The mixture basified to pH 8 with saturated NaHCO₃ (aq.). The resulting mixture was extracted with EtOAc (3×500 mL). The combined organic layers were washed with brine (500 mL), dried over anhydrous Na₂SO₄. After filtration, the filtrate was concentrated under reduced pressure. The residue was purified by silica gel column chromatography, eluted with PE/EA (2:1) to afford the title compound as a yellow solid (9.8 g, 40.2%). LCMS: m/z=228 [M+H]⁺

### Preparation 13—2-chloro-5,7-dimethylpyrido[2,3-d]pyrimidin-4-amine

A mixture of 2,4-dichloro-5,7-dimethylpyrido[2,3-d]pyrimidine (6.00 g, 26.3 mmol) in ammonium hydroxide (90.0 mL) was stirred for 1 h at room temperature. The resulting mixture was concentrated under reduced pressure. The residue was purified by Prep-HPLC^{B} to afford title compound as an off-white solid (4.45 g, 81.1%). LCMS m/z=209 [M+H]⁺

### Preparation 14—5,7-dimethyl-N²-(1-methylpiperidin-4-yl)pyrido[2,3-d]pyrimidine-2,4-diamine

To a stirred solution of 2-chloro-5,7-dimethylpyrido[2,3-d]pyrimidin-4-amine (60.0 mg, 0.288 mmol) in dioxane (1.50 mL) at room temperature was added 1-methylpiperidin-4-amine (164 mg, 1.44 mmol). The resulting mixture was stirred at 80° C. overnight. The mixture was allowed to cool down to room temperature. The resulting mixture was concentrated under reduced pressure. The residue was dissolved in DMF (2.00 mL). The crude product was purified by Prep-HPLC^{AU} to afford the title compound as an off-white solid (5.5 mg, 6.52%). ¹H NMR (400 MHz, DMSO-d⁶) δ 6.67 (s, 3H), 6.38 (s, 1H), 3.84-3.69 (m, 1H), 2.74 (d, J=11.6 Hz, 2H), 2.63 (s, 3H), 2.37 (s, 3H), 2.16 (s, 3H), 2.02-1.88 (m, 2H), 1.81-1.78 (m, 2H), 1.56-1.48 (m, 2H). LCMS^{H}: m/z=287 [M+H]⁺.

### Example 6: 5,7-dimethyl-N²-(3-morpholinopropyl)pyrido[2,3-d]pyrimidine-2,4-diamine

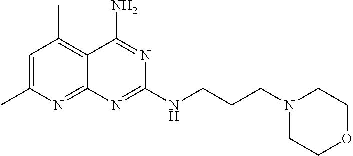 [SMILES: Cc1cc(C)c2c(N)nc(NCCCN3CCOCC3)nc2n1]

To a stirred mixture of 2-(cyanoamino)-4,6-dimethylpyridine-3-carbonitrile (100 mg, 0.581 mmol) in EtOH (1.5 mL) at room temperature was added 4-morpholinepropanamine (100 mg, 0.697 mmol). The resulting mixture was stirred at 80° C. overnight. The resulting mixture was concentrated under reduced pressure. The crude product was purified by Prep-HPLC^{H} to afford the title compound as a white solid (79 mg, 42.9%). ¹H NMR (300 MHz, MeOD) δ 6.78 (s, 1H), 3.70 (t, J=4.7 Hz, 4H), 3.48 (t, J=6.7 Hz, 2H), 2.71 (s, 3H), 2.53-2.42 (m, 9H), 1.87-1.77 (m, 2H). LCMS^{C}: m/z=317 [M+H]⁺.

### Example 7: 5,7-dimethyl-N²-(3-(piperidin-1-yl)propyl)pyrido[2,3-d]pyrimidine-2,4-diamine

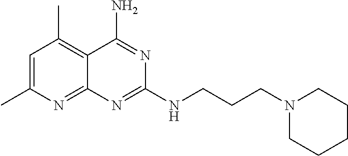 [SMILES: Cc1cc(C)c2c(N)nc(NCCCN3CCCCC3)nc2n1]

As described for Example 6, using 3-(piperidin-1-yl) propan-1-amine and purification by Prep-HPLC^{L} to afford the title compound as a white solid (90 mg, 43.5%). 1H NMR (400 MHz, MeOD) δ 6.79 (s, 1H), 3.48-3.45 (m, 2H), 2.71-2.62 (m, 9H), 2.46 (s, 3H), 1.92-1.87 (m, 2H), 1.69-1.60 (m, 4H). 1.60-1.51 (m, 2H). LCMS^{E} m/z=315 [M+H]⁺.

### Example 8: 4-(3-((4-amino-5,7-dimethylpyrido[2,3-d]pyrimidin-2-yl)amino)propyl)thiomorpholine 1,1-dioxide

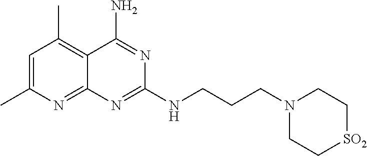 [SMILES: Cc1cc(C)c2c(N)nc(NCCCN3CCS(=O)(=O)CC3)nc2n1]

As described for Example 6, using 4-(3-aminopropyl) thiomorpholine 1,1-dioxide and purification by Prep-HPLC^{J} to afford the title compound as a white solid (46.9 mg, 44.3%). ¹H NMR (400 MHz, MeOD) δ 6.80 (s, 1H), 3.51 (t, J=6.0 Hz, 2H), 3.14-3.08 (m, 4H), 3.01-2.96 (m, 4H), 2.72 (s, 3H), 2.63 (t, J=8.0 Hz, 2H), 2.47 (s, 3H), 1.86-1.75 (m, 2H). LCMS^{A}: m/z=365 [M+H]⁺

### Example 9: 6-methyl-N³-(1-methylpiperidin-4-yl)pyrimido[4,5-c]isoquinoline-1,3-diamine

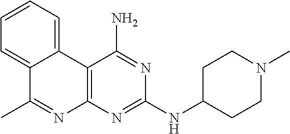

### Route A

### Preparation 15—1-methyl-3-oxo-3,4-dihydroisoquinoline-4-carbonitrile and 2-hydroxy-4-methylquinoline-3-carbonitrile

To a solution of 1-(2-fluorophenyl)ethanone (25.0 g, 181 mmol) and cyanoacetamide (33.5 g, 398 mmol) in DMSO (250 mL) were added NaH (16.7 g, 416 mmol, 60% wt in mineral oil) in portions at 0° C. The resulting mixture was stirred at 80° C. overnight. The mixture was allowed to cool down to room temperature. The mixture was acidified to pH 3 with cold HCl (aq., 1 M). The precipitated solids were collected by filtration and washed with water (3×50 mL). The solid was dried under vacuum to afford the title compound (19.0 g, crude) as a yellow solid. The crude product (6.0 g) was further purified by Prep-HPLC^{B} to afford 1-methyl-3-oxo-3,4-dihydroisoquinoline-4-carbonitrile (1.78 g) and 2-hydroxy-4-methylquinoline-3-carbonitrile (756 mg). LCMS m/z=185 [M+H]⁺

### Preparation 16—4-cyano-1-methylisoquinolin-3-yl trifluoromethanesulfonate

To a stirred solution of 1-methyl-3-oxo-3,4-dihydroisoquinoline-4-carbonitrile (100 mg, 0.543 mmol) and Et₃N (151 μL, 1.09 mmol) in DCM (2.0 mL) was added Tf₂O (183 uL, 1.09 mmol) dropwise at 0° C. The resulting mixture was stirred under nitrogen atmosphere at room temperature for 1 h. The resulting mixture was concentrated under reduced pressure. The residue was purified by Prep-TLC (PE/EA 10:1) to afford the title compound (130 mg, 75.7%) as a yellow solid. LCMS: m/z=315 [M−H]⁺

### Preparation 17—N-(4-cyano-1-methylisoquinolin-3-yl)cyanamide

To a stirred solution of 4-cyano-1-methylisoquinolin-3-yl trifluoromethanesulfonate (100 mg, 0.316 mmol) and cyanamide (26.6 mg, 0.632 mmol) in 1,4-dioxane (1.5 mL) were added Xantphos (18.3 mg, 0.032 mmol), Pd₂(dba)₃ (29.0 mg, 0.032 mmol) and DIEA (110 μL, 0.632 mmol) under nitrogen atmosphere at room temperature. The resulting mixture was stirred under nitrogen atmosphere at 100° C. for 1 h. The mixture was allowed to cool down to room temperature and diluted with water/ice (10 mL). The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/EA (5:1) to afford the title compound (45.0 mg, 68.4%) as a yellow solid. LCMS m/z=209 [M+H]⁺

### Preparation 18—6-methyl-N³-(1-methylpiperidin-4-yl)pyrimido[4,5-c]isoquinoline-1,3-diamine

A solution of N-(4-cyano-1-methylisoquinolin-3-yl)cyanamide (40 mg, 0.192 mmol) and 1-methylpiperidin-4-amine (43.9 mg, 0.384 mmol) in EtOH (1.0 mL) was stirred at 80° C. overnight. The mixture was allowed to cool down to room temperature. The residue was purified by Prep-HPLC^{B} the title compound (7.70 mg, 10.9%) as a yellow solid formate salt. ¹H NMR (400 MHz, DMSO-d₆) δ 8.51 (d, J=8.5 Hz, 1H), 8.29 (s, 1H), 8.20 (d, J=8.2 Hz, 1H), 7.77 (t, J=7.4 Hz, 1H), 7.50 (t, J=7.6 Hz, 1H), 6.98 (s, 2H), 6.58 (s, 1H), 3.87-3.80 (m, 1H), 2.87 (s, 3H), 2.81 (d, J=11.6 Hz, 2H), 2.21 (s, 3H), 2.06 (t, J=10.1 Hz, 2H), 1.92-1.82 (m, 2H), 1.61-1.46 (m, 2H). LCMS^{|}: m/z=323 [M+H]⁺

### Example 10: 5-methyl-N²-(1-methylpiperidin-4-yl)pyrimido[4,5-b]quinoline-2,4-diamine

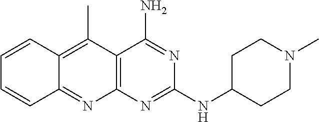 [SMILES: Cc1c2ccccc2nc2nc(NC3CCN(C)CC3)nc(N)c12]

### Route A

### Preparation 19—2-chloro-4-methylquinoline-3-carbonitrile

To a solution of 2-hydroxy-4-methylquinoline-3-carbonitrile (1.00 g, 5.43 mmol, synthesised as described in Example 9, Preparation 15) in 1,4-dioxane (15.0 mL) was added phosphorus oxychloride (8.30 g, 54.3 mmol) dropwise at room temperature. The resulting mixture was stirred at 100° C. for 6 h. The mixture was allowed to cool down to room temperature. The reaction was quenched with sat. NaHCO₃(aq.) at 0° C. The resulting mixture was extracted with CH₂Cl₂ (3×100 mL). The combined organic layers were washed with brine (1×50 mL), dried over anhydrous Na₂SO₄ filtered and concentrated under reduced pressure. The residue was purified by silica gel column chromatography (eluted with PE/EA (1:1)) to afford the title compound (260 mg, 23.6%) as a white solid. LCMS: m/z=203 [M+H]⁺

### Preparation 20—N-(3-cyano-4-methylquinolin-2-yl)cyanamide

To a solution of 2-chloro-4-methylquinoline-3-carbonitrile (230 mg, 1.14 mmol), cyanamide (95.0 mg, 2.27 mmol) in 1,4-dioxane (6.0 mL) were added BrettPhos Pd G3 (103 mg, 0.114 mmol), BrettPhos (61.0 mg, 0.114 mmol) and Cs₂CO₃ (740 mg, 2.27 mmol). The mixture was stirred at 100° C. under nitrogen atmosphere for 1 h. The mixture was allowed to cool down to room temperature. The resulting mixture was filtered, the filter cake was washed with MeOH (3×10 mL). The filtrate was concentrated under reduced pressure. The residue was purified by Prep-HPLC^{B} to afford the title compound (129 mg, 54.6%) as a yellow solid. LCMS: m/z=209 [M+H]⁺

### Preparation 21—5-methyl-N²-(1-methylpiperidin-4-yl)pyrimido[4,5-b]quinoline-2,4-diamine

A mixture of N-(3-cyano-4-methylquinolin-2-yl)cyanamide (110 mg, 0.528 mmol) and 1-methylpiperidin-4-amine (72.0 mg, 0.634 mmol) in 1,4-dioxane (2.5 mL) was stirred at 100° C. for 1 h. The mixture was allowed to cool down to room temperature. The resulting mixture was concentrated under reduced pressure. The residue was purified by Prep-HPLC® to afford the title compound (26.9 mg, 15.8%) as a yellow solid. ¹H NMR (400 MHz, DMSO-d₆) δ 7.91 (d, J=8.0 Hz, 1H), 7.69-7.54 (m, 2H), 7.37-7.29 (m, 1H), 3.77 (br, 1H), 2.75 (s, 3H), 2.73-2.63 (m, 2H), 2.17 (s, 3H), 2.10-1.99 (m, 2H), 1.99-1.87 (m, 2H), 1.60-1.33 (m, 2H). LCMS^{I}: m/z=323 [M+H]⁺

### Route B

### Preparation 22—2-hydroxy-4-methylquinoline-3-carbonitrile

To a solution of o-aminoacetophenone (5.00 g, 37.0 mmol) and cyanoacetic acid (4.70 g, 55.2 mmol) in THF (25 mL) were added HOBt (497 mg, 3.69 mmol), EDCl (12.7 g, 66.1 mmol) and DIEA (9.47 g, 74.0 mmol) at room temperature. The resulting mixture was stirred at room temperature overnight. The resulting mixture was diluted with water (100 mL) and extracted with EtOAc (3×100 mL). The combined organic layers were washed with brine (100 mL), dried over anhydrous Na₂SO₄, filtered, and concentrated under reduced pressure. The crude product was re-crystallized from DMSO/water (200 mL, 1/1) to afford the title compound (3.27 g, 48.0%) as a white solid. LCMS m/z: 185 [M+H]⁺

### Preparation 23—2-chloro-4-methylquinoline-3-carbonitrile

To a solution of 2-hydroxy-4-methylquinoline-3-carbonitrile (1.50 g, 8.14 mmol) in dioxane (30 mL) was added POCl₃ (12.5 g, 81.4 mmol) over 5 min at 0° C. The resulting mixture was stirred at 100° C. overnight. The resulting mixture was concentrated under reduced pressure. The residue was dissolved in DCM (30 mL) and quenched by the addition of sat. NaHCO₃ (aq.) (30 mL) at 0° C. and extracted with DCM (3×30 mL). The combined organic phases were washed with brine (30 mL), dried over Na₂SO₄, filtered, and concentrated under reduced pressure. This resulted in the title compound (1.40 g, crude) as a brown solid. LCMS m/z=203 [M+H]⁺.

### Preparation 24—2-(cyanoamino)-4-methylquinoline-3-carbonitrile

To a solution of 2-chloro-4-methylquinoline-3-carbonitrile (800 mg, 3.95 mmol) in DMSO (20 mL) was added cyanamide, monosodium salt (508 mg, 7.94 mmol). The reaction mixture was stirred at 80° C. overnight. The mixture was allowed to cool down to room temperature and diluted with water (50 mL). The resulting mixture was extracted with EtOAc (50 mL×3). The combined organic phases were washed with brine (50 mL), dried over Na₂SO₄, filtered, and concentrated under reduced pressure. The residue was purified by Prep-HPLC^{B} to afford the title compound (390 mg, 47.4%) as a light-yellow solid. LCMS m/z=209 [M+H]⁺.

### Preparation 25—5-methyl-N²-(1-methylpiperidin-4-yl)pyrimido[4,5-b]quinoline-2,4-diamine

To a solution of 2-(cyanoamino)-4-methylquinoline-3-carbonitrile (390 mg, 1.87 mmol) in dioxane (20 mL) was added 1-methylpiperidin-4-amine (937 mg, 8.23 mmol). The resulting mixture was stirred at 100° C. for 2 h. The mixture was allowed to cool down to room temperature and concentrated under reduced pressure. The residue was purified by Prep-HPLC^{B} to afford the title compound (212.3 mg, 35.2%) as a yellow solid. ¹H NMR (400 MHz, DMSO-d₆) δ 7.92 (d, J=8.3 Hz, 1H), 7.68-7.57 (m, 2H), 7.33 (t, J=7.5 Hz, 1H), 3.78 (br, 1H), 2.75 (s, 3H), 2.69 (d, J=10.1 Hz, 2H), 2.18 (s, 3H), 2.09-2.01 (m, 2H), 1.95 (d, J=12.4 Hz, 2H), 1.46 (s, 2H). LCMS' m/z=323 [M+H]⁺.

### Example 11: 6-methyl-N³-(1-methylpiperidin-4-yl)-8,9-dihydro-7H-cyclopenta[4,5]pyrido[2,3-d]pyrimidine-1,3-diamine

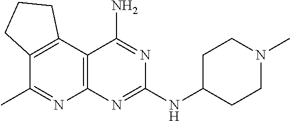

### Preparation 26—Mixture of 1-methyl-3-oxo-4,5,6,7-tetrahydro-3H-cyclopenta[c]pyridine-4-carbonitrile and 4-methyl-2-oxo-3,5,6,7-tetrahydro-2H-cyclopenta[b]pyridine-3-carbonitrile

To a mixture of 2-acetylcyclopentan-1-one (2.00 g, 15.9 mmol) and 2-cyanoacetamide (1.33 g, 15.9 mmol) in ethanol (20.0 mL) was added piperidine (1.35 g, 15.9 mmol). The mixture was stirred at 80° C. for 6 h. The mixture was allowed to cool down to room temperature. The precipitated solids were collected by filtration and washed with ethanol (3×50 mL). The solid was dried under vacuum to afford the title compound (1.67 g, crude, mixture of regioisomers) as a white solid. LCMS m/z=175 [M+H]⁺.

### Preparation 27—3-chloro-1-methyl-6,7-dihydro-5H-cyclopenta[c]pyridine-4-carbonitrile and 2-chloro-4-methyl-6,7-dihydro-5H-cyclopenta[b]pyridine-3-carbonitrile

To a solution of a mixture of 1-methyl-3-oxo-4,5,6,7-tetrahydro-3H-cyclopenta[c]pyridine-4-carbonitrile and 4-methyl-2-oxo-3,5,6,7-tetrahydro-2H-cyclopenta[b]pyridine-3-carbonitrile (800 mg, 4.59 mmol) in dioxane (15.0 mL) was added POCl₃ (7.04 g, 45.9 mmol) dropwise at 0° C. The resulting mixture was stirred at 100° C. for 6 h. The mixture was allowed to cool down to room temperature and concentrated under reduced pressure. The resulting mixture was diluted with CH₂Cl₂ (20 mL) and quenched with sat. NaHCO₃ (aq.) at 0° C. The resulting mixture was extracted with CH₂Cl₂ (3×80 mL). The combined organic layers were washed with brine (40 mL), dried over anhydrous Na₂SO₄, filtered, and concentrated under reduced pressure. The residue was purified by silica gel column chromatography, eluted with PE/EA (5:1) to afford a mixture of two isomers (770 mg, 87.0%) as a white solid. The mixture was separated by SFC^{A} to afford 3-chloro-1-methyl-6,7-dihydro-5H-cyclopenta[c]pyridine-4-carbonitrile (390 mg, R_{f}=3 min) as a white solid and 2-chloro-4-methyl-6,7-dihydro-5H-cyclopenta[b]pyridine-3-carbonitrile (250 mg, R_{f}=4 min) as a white solid. LCMS m/z=193 [M+H]⁺

### Preparation 28—N-(4-cyano-1-methyl-6,7-dihydro-5H-cyclopenta[c]pyridin-3-yl)cyanamide

To a solution of 3-chloro-1-methyl-6,7-dihydro-5H-cyclopenta[c]pyridine-4-carbonitrile (360 mg, 1.87 mmol) and cyanamide (86.4 mg, 2.06 mmol) in dioxane (8.0 mL) were added BrettPhos Pd G3 (169 mg, 0.187 mmol), BrettPhos (100 mg, 0.187 mmol) and Cs₂CO₃ (1.21 g, 3.74 mmol). The mixture was stirred under nitrogen atmosphere at 100° C. for 1 h. The mixture was allowed to cool down to room temperature. The resulting mixture was filtered through a short pad of Celite. The pad was washed with CH₂Cl₂ (3×50 mL). The combined filtrate was concentrated under reduced pressure. The residue was purified by Prep-HPLC^{B} to afford the title compound (150 mg, 40.5%) as a white solid. LCMS m/z=199 [M+H]⁺.

### Preparation 29—6-methyl-N³-(1-methylpiperidin-4-yl)-8,9-dihydro-7H-cyclopenta[4,5]pyrido[2,3-d]pyrimidine-1,3-diamine

A solution of N-(4-cyano-1-methyl-6,7-dihydro-5H-cyclopenta[c]pyridin-3-yl)cyanamide (60 mg, 0.303 mmol) and 1-methylpiperidin-4-amine (41.5 mg, 0.364 mmol) in EtOH (1.5 mL) was stirred for 3 h at 80° C. The mixture was allowed to cool down to room temperature and concentrated under reduced pressure. The residue was purified by Prep-HPLC^{B} to afford the title compound as a white solid. ¹H NMR (400 MHz, DMSO-d₆) δ 6.56 (s, 2H), 6.27 (s, 1H), 3.86-3.62 (m, 1H), 2.85-2.70 (m, 4H), 2.37 (s, 3H), 2.16 (s, 3H), 2.13-2.03 (m, 2H), 1.94 (t, J=11.5 Hz, 2H), 1.87-1.73 (m, 2H), 1.57-1.44 (m, 2H). LCMS^{A} m/z=313 [M+H]⁺

### Example 12: 5-methyl-N²-(1-methylpiperidin-4-yl)-7,8-dihydro-6H-cyclopenta[5,6]pyrido[2,3-d]pyrimidine-2,4-diamine

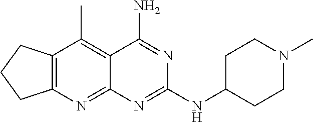 [SMILES: Cc1c2c(nc3nc(NC4CCN(C)CC4)nc(N)c13)CCC2]

### Preparation 30—N-(3-cyano-4-methyl-6,7-dihydro-5H-cyclopenta[b]pyridin-2-yl)cyanamide

To the solution of 2-chloro-4-methyl-6,7-dihydro-5H-cyclopenta[b]pyridine-3-carbonitrile (230 mg, 1.194 mmol, synthesised as described in Example 11, Preparation 27) and cyanamide (55.2 mg, 1.31 mmol) in dioxane (5.0 mL) were added BrettPhos Pd G3 (108 mg, 0.119 mmol), BrettPhos (64.1 mg, 0.119 mmol) and Cs₂CO₃ (778 mg, 2.39 mmol). The mixture was stirred at 100° C. under nitrogen atmosphere for 1 h. The mixture was allowed to cool down to room temperature. The resulting mixture was filtered through a short pad of Celite. The pad was washed with CH₂Cl₂ (2×10 mL). The combined filtrate was concentrated under reduced pressure. The residue was purified by Prep-HPLC^{B} to afford the title compound (120 mg, 50.7%) as a yellow solid. LCMS m/z=199 [M+H]⁺.

### Preparation 31—5-methyl-N2-(1-methylpiperidin-4-yl)-7,8-dihydro-6H-cyclopenta[5,6]pyrido[2,3-d]pyrimidine-2,4-diamine

A mixture of N-(3-cyano-4-methyl-6,7-dihydro-5H-cyclopenta[b]pyridin-2-yl)cyanamide (60.0 mg, 0.303 mmol) and 1-methylpiperidin-4-amine (51.9 mg, 0.455 mmol) in EtOH (1.5 mL) was stirred for 5 h at 80° C. The mixture was allowed to cool down to room temperature. The resulting mixture was filtered through a short pad of Celite. The pad was washed with CH₂Cl₂ (3×10 mL). The combined filtrate was concentrated under reduced pressure. The residue was purified by Prep-HPLC^{B} to afford the title compound (31.3 mg, 33.1%) as a white solid. ¹H NMR (400 MHz, DMSO-d₆) δ 6.63 (s, 2H), 6.26 (s, 1H), 3.82-3.67 (m, 1H), 2.88-2.86 (m, 4H), 2.75-2.71 (m, 2H), 2.57 (s, 3H), 2.16 (s, 3H), 2.07-1.99 (m, 2H), 1.98-1.87 (m, 2H), 1.86-1.81 (m, 2H), 1.57-1.42 (m, 2H). LCMS^{O} m/z=313 [M+H]⁺

### Example 13: 7-methyl-N²-(1-methylpiperidin-4-yl)-5-phenylpyrido[2,3-d]pyrimidine-2,4-diamine

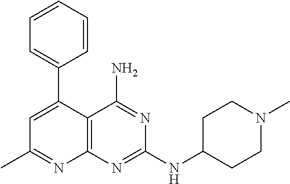

### Preparation 32—2-hydroxy-6-methyl-4-phenylpyridine-3-carbonitrile

To a solution of 4-phenylbut-3-en-2-one (2.00 g, 13.7 mmol) and cyanoacetamide (1.38 g, 16.4 mmol) in DMSO (2.00 mL) was added t-BuOK (6.14 g, 54.7 mmol). The resulting mixture was stirred at 60° C. overnight. The resulting mixture was filtered through a short pad of Celite. The pad was washed with EtOAc (10 mL). The combined filtrate was diluted with water (10 mL) and extracted with EtOAc (10 mL×3). The combined organic phases were washed with brine (10 mL), dried over Na₂SO₄, filtered, and concentrated under reduce pressure. The residue was purified by Prep-HPLC^{A} to afford the title compound as a white solid (210 mg, 7.3%). LCMS m/z=211 [M+H]⁺.

### Preparation 33—2-chloro-6-methyl-4-phenylpyridine-3-carbonitrile

The solution of 2-hydroxy-6-methyl-4-phenylpyridine-3-carbonitrile (200 mg, 0.952 mmol) in POCl₃ (3.0 mL) was stirred at 100° C. overnight. The resulting mixture was concentrated under reduce pressure. The residue was purified by Prep-HPLC^{S} to afford the title compound as a yellow solid (120 mg, 55.2%). LCMS m/z=229 [M+H]⁺.

### Preparation 34—2-(cyanoamino)-6-methyl-4-phenylpyridine-3-carbonitrile

To a solution of 2-chloro-6-methyl-4-phenylpyridine-3-carbonitrile (110 mg, 0.481 mmol) in DMSO (3.0 mL) at room temperature was added cyanamide, monosodium salt (61.6 mg, 0.962 mmol, 2.00 equiv). The resulting mixture was stirred 100° C. overnight. The reaction mixture was allowed to cool to room temperature and quenched by the addition of water (10 mL). The mixture was extracted with EtOAc (10 mL×3). The combined organic phases were washed with brine (10 mL), dried over Na₂SO₄, filtered, and concentrated under reduce pressure. The residue was further purified by Prep-HPLC^{S} to afford the title compound as a white solid (60.0 mg, 53.2%). LCMS m/z=235 [M+H]⁺.

### Preparation 35—7-methyl-N²-(1-methylpiperidin-4-yl)-5-phenylpyrido[2,3-d]pyrimidine-2,4-diamine

To a solution of 2-(cyanoamino)-6-methyl-4-phenylpyridine-3-carbonitrile (50.0 mg, 0.213 mmol) in dioxane (1.5 mL) at room temperature was added 1-methylpiperidin-4-amine (29.2 mg, 0.260 mmol). The resulting mixture was stirred at 80° C. overnight. The resulting mixture was concentrated under reduced pressure. The residue was purified by Prep-HPLC^{T} to afford the title compound as a white solid (18.5 mg, 24.6%). ¹H NMR (400 MHz, DMSO-d₆) δ 7.63-7.49 (m, 3H), 7.47-7.37 (m, 2H), 6.67 (s, 1H), 6.58 (s, 1H), 3.90-3.73 (m, 1H), 2.76-2.73 (m, 2H), 2.46 (s, 3H), 2.16 (s, 3H), 1.95-1.92 (m, 2H), 1.83-1.80 (m, 2H), 1.59-1.43 (m, 2H). LCMS m/z=349 [M+H]⁺.

### Example 14: (R)-5,7-dimethyl-N²-(1-methylpyrrolidin-3-yl)pyrido[2,3-d]pyrimidine-2,4-diamine

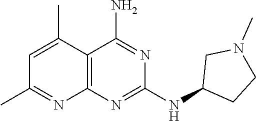 [SMILES: Cc1cc(C)c2c(N)nc(N[C@@H]3CCN(C)C3)nc2n1]

To a stirred solution of 2-(cyanoamino)-4,6-dimethylpyridine-3-carbonitrile (100 mg, 0.581 mmol, and (3R)-1-methylpyrrolidin-3-amine dihydrochloride (121 mg, 0.697 mmol) in EtOH (4.0 mL) was added DIEA (150 mg, 1.16 mmol) at room temperature. The resulting mixture was stirred at 80° C. overnight. The resulting mixture was concentrated under reduced pressure. The residue was purified by Prep-HPLC^{AN} to afford the title compound (62.4 mg, 39.5%) as a white solid. ¹H NMR (400 MHz, Methanol-d₄) δ 6.81 (s, 1H), 4.67-4.57 (m, 1H), 2.98 (t, J=8.8 Hz, 1H), 2.92-2.75 (m, 1H), 2.72 (s, 3H), 2.66-2.54 (m, 2H), 2.47 (s, 3H), 2.45-2.31 (m, 4H), 1.85-1.74 (m, 1H). LCMS^{H} m/z=273 [M+H]⁺

### Example 15: (S)-5,7-dimethyl-N²-(1-methylpyrrolidin-3-yl)pyrido[2,3-d]pyrimidine-2,4-diamine

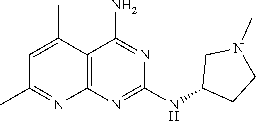 [SMILES: Cc1cc(C)c2c(N)nc(N[C@H]3CCN(C)C3)nc2n1]

As described for Example 14 using (3S)-1-methylpyrrolidin-3-amine dihydrochloride and purification by Prep-HPLC^{U} to afford the title compound (41.6 mg, 26.3%) as a white solid. ¹H NMR (400 MHz, DMSO-d₆) δ 6.95-6.46 (m, 4H), 4.48-4.35 (m, 1H), 2.88-2.72 (m, 1H), 2.64 (s, 3H), 2.58-2.54 (m, 1H), 2.47-2.41 (m, 1H), 2.41-2.31 (m, 4H), 2.26 (s, 3H), 2.20-2.08 (m, 1H), 1.78-1.63 (m, 1H). LCMS^{H} m/z=273 [M+H]⁺.

### Example 16: 5-methyl-N²-(1-methylpiperidin-4-yl)-7-phenylpyrido[2,3-d]pyrimidine-2,4-diamine

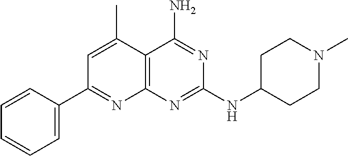 [SMILES: Cc1cc(-c2ccccc2)nc2nc(NC3CCN(C)CC3)nc(N)c12]

### Preparation 36—2-chloro-4-methyl-6-phenylpyridine-3-carbonitrile

To a solution of 2,6-dichloro-4-methylpyridine-3-carbonitrile (1.00 g, 5.37 mmol) and phenyl boronic acid (521 mg, 4.27 mmol) in DME (5.0 mL) and H₂O (1.0 mL) were added Pd(PPh₃)₄ (617 mg, 0.530 mmol) and Na₂CO₃ (1.13 g, 10.6 mmol). The mixture was irradiated with microwave radiation under nitrogen atmosphere at 80° C. for 1 h. The mixture was allowed to cool down to room temperature and quenched by the addition of water (50 mL). The resulting mixture was extracted with EtOAc (3×50 mL). The combined organic layers were washed with brine (2×30 mL), dried over anhydrous Na₂SO₄, filtered, and concentrated under reduced pressure. The residue was purified by Prep-HPLC^{L} to afford the title compound (420 mg, 34.3%) as a white solid. LCMS m/z=229 [M+H]⁺

### Preparation 37—2-(cyanoamino)-4-methyl-6-phenylpyridine-3-carbonitrile

To a mixture of 2-chloro-4-methyl-6-phenylpyridine-3-carbonitrile (410 mg, 1.79 mmol) and aminoformonitrile (150 mg, 3.58 mmol) in dioxane (5.0 mL) were added Brettphos Pd G3 (162 mg, 0.170 mmol), Brettphos (96.2 mg, 0.170 mmol) and Cs₂CO₃ (1.16 g, 3.58 mmol). The mixture was stirred under nitrogen atmosphere at 100° C. for 2 h. The mixture was allowed to cool down to room temperature. The resulting mixture was filtered through a short pad of Celite. The pad was washed with DCM (30 mL). The combined filtrated was concentrated under reduced pressure. The residue was purified by Prep-HPLC^{L} to afford the title compound (302 mg, 71.9%) as a yellow solid. LCMS m/z=235 [M+H]⁺.

### Preparation 38—5-methyl-N²-(1-methylpiperidin-4-yl)-7-phenylpyrido[2,3-d]pyrimidine-2,4-diamine

A solution of 2-(cyanoamino)-4-methyl-6-phenylpyridine-3-carbonitrile (190 mg, 0.811 mmol) and 1-methylpiperidin-4-amine (648 mg, 5.67 mmol) in DMSO (2.0 mL) was irradiated with microwave radiation at 180° C. for 2 h. The mixture was allowed to cool down to room temperature. The resulting mixture was filtered through a short pad of Celite. The pad was washed with EtOAc (10 mL). The combined filtrate was concentrated under reduce pressure. The residue was purified by Prep-HPLC^{W} to afford the title compound (26.6 mg, 9.41%) as a white solid. ¹H NMR (400 MHz, DMSO-d⁶) δ 8.19-8.08 (m, 2H), 7.55-7.45 (m, 3H), 7.42 (s, 1H), 6.76 (br, 2H), 6.44 (br, 1H), 3.93-3.78 (m, 1H), 2.82-2.70 (m, 5H), 2.17 (s, 3H), 1.98 (t, J=12.0 Hz, 2H), 1.82 (d, J=12.0 Hz, 2H), 1.60-1.45 (m, 2H). LCMS^{C} m/z=349 [M+H]⁺

### Example 17: 7-methoxy-N²-(1-methylpiperidin-4-yl)pyrido[2,3-d]pyrimidine-2,4-diamine

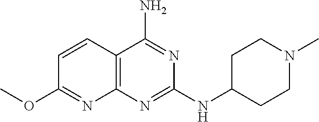

### Preparation 39—2-(cyanoamino)-6-methoxypyridine-3-carbonitrile

To a mixture of 2-bromo-6-methoxypyridine-3-carbonitrile (300 mg, 1.41 mmol) in dioxane (3.00 mL) was added cyanamide (118 mg, 2.82 mmol), Brettphos Pd G3 (1.28 g, 1.41 mmol), Brettphos (756 mg, 1.41 mmol) and K₂CO₃ (389 mg, 2.82 mmol) at room temperature. The resulting mixture was stirred for 2 h at 80° C. under nitrogen atmosphere. The mixture was allowed to cool down to room temperature. The resulting mixture was filtered through a short pad of Celite. The pad was washed with EtOAc (5 mL). The combined filtrate was concentrated under reduced pressure. The residue was purified by Prep-HPLC^{B} to afford the title compound as white solid (130 mg, 53.0%). LCMS m/z=175 [M+H]⁺

### Preparation 40—7-methoxy-N²-(1-methylpiperidin-4-yl)pyrido[2,3-d]pyrimidine-2,4-diamine

To a solution of 2-(cyanoamino)-6-methoxypyridine-3-carbonitrile (130 mg, 0.746 mmol) in dioxane (1.30 mL) was added 1-methylpiperidin-4-amine (102 mg, 0.895 mmol) at room temperature. The resulting mixture was stirred at 80° C. for 6 h. The mixture was allowed to cool down to room temperature. The crude product was purified by Prep-HPLC^{I} to afford the title compound (95.6 mg, 44.4%). ¹H NMR (400 MHz, DMSO-d₆) δ 8.21 (d, J=8.6 Hz, 1H), 7.41-6.99 (br, 2H), 6.43 (d, J=8.6 Hz, 2H), 3.87 (br, 4H), 2.73 (d, J=10.5 Hz, 2H), 2.16 (s, 3H), 1.96 (t, J=11.6 Hz, 2H), 1.86-1.75 (m, 2H), 1.51 (m, 2H). LCMS^{H} m/z=289 [M+H]⁺

### Example 18: N²-(1-methylpiperidin-4-yl)-7-(trifluoromethyl)pyrido[2,3-d]pyrimidine-2,4-diamine

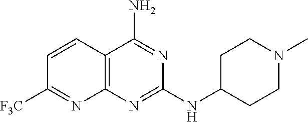 [SMILES: CN1CCC(Nc2nc(N)c3ccc(C(F)(F)F)nc3n2)CC1]

### Preparation 41—2-(cyanoamino)-6-(trifluoromethyl)pyridine-3-carbonitrile

To a mixture of 2-chloro-6-(trifluoromethyl)pyridine-3-carbonitrile (1.00 g, 4.84 mmol) in DMSO (10.0 mL) was added cyanamide, monosodium salt (619 mg, 9.68 mmol). The mixture was stirred for 2 h at 100° C. The resulting mixture was concentrated under reduced pressure. The residue was purified by Prep-HPLC^{B} to afford the title compound as a light brown solid (618 mg, 60.1%). LCMS m/z=211 [M−H]⁻

### Preparation 42—N²-(1-methylpiperidin-4-yl)-7-(trifluoromethyl)pyrido[2,3-d]pyrimidine-2,4-diamine

To a mixture of 2-(cyanoamino)-6-(trifluoromethyl)pyridine-3-carbonitrile (200 mg, 0.943 mmol, 1.00 equiv) in dioxane (5.00 mL) was added 1-methylpiperidin-4-amine (215 mg, 1.88 mmol, 2.00 equiv). The mixture was stirred for 2 h at 100° C. The resulting mixture was concentrated under reduced pressure. The crude product was purified by Prep-HPLC^{W} to afford the title compound as a white solid (182.9 mg, 59.5%). ¹H NMR (400 MHz, Methanol-d₄) δ 8.49 (d, J=8.1 Hz, 1H), 7.39 (d, J=8.1 Hz, 1H), 4.84 (s, 1H), 3.98 (dt, J=11.2, 6.7 Hz, 1H), 2.87 (d, J=11.7 Hz, 2H), 2.45-2.13 (m, 5H), 2.05 (d, J=13.7 Hz, 2H), 1.60-1.59 (m, 2H). LCMS^{H} m/z=327 [M+H]⁺

### Example 19: N²-(1-methylpiperidin-4-yl)-6-(trifluoromethyl)pyrido[2,3-d]pyrimidine-2,4-diamine

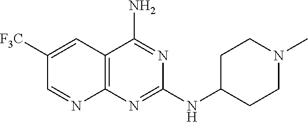 [SMILES: CN1CCC(Nc2nc(N)c3cc(C(F)(F)F)cnc3n2)CC1]

### Preparation 43—2-(cyanoamino)-5-(trifluoromethyl)pyridine-3-carbonitrile

A mixture of 2-chloro-5-(trifluoromethyl)pyridine-3-carbonitrile (500 mg, 2.42 mmol) and cyanamide, monosodium salt (309 mg, 4.84 mmol) in DMSO (5.0 mL) was stirred at 100° C. for 1 h. The mixture was allowed to cool down to room temperature and acidified to pH 1 with aq. HCl (6.0 M). The precipitated solids were collected by filtration and dried under vacuum to afford the title compound (200 mg crude) as a grey solid. LCMS m/z=213 [M+H]⁺

### Preparation 44—N²-(1-methylpiperidin-4-yl)-6-(trifluoromethyl)pyrido[2,3-d]pyrimidine-2,4-diamine

A mixture of 2-(cyanoamino)-5-(trifluoromethyl)pyridine-3-carbonitrile (195 mg, 0.919 mmol) and 1-methylpiperidin-4-amine (209 mg, 1.83 mmol) in dioxane (3.0 mL) was stirred at 100° C. for 1 h. The mixture was allowed to cool down to room temperature and concentrated under reduced pressure. The crude product was purified by Prep-HPLC to afford the title compound (142 mg, 47.3%) as a white solid. ¹H NMR (400 MHz, DMSO-d⁶) δ 9.06-8.65 (m, 2H), 8.36-7.45 (m, 2H), 7.45-7.00 (m, 1H), 3.87 (s, 1H), 2.94-2.70 (m, 2H), 2.37 (m, 3H), 2.17-1.96 (m, 2H), 1.93-1.75 (m, 2H), 1.66-1.47 (m, 2H). LCMS^{C} m/z=327 [M+H]⁺

### Example 20: 6-bromo-N²-(1-methylpiperidin-4-yl)pyrido[2,3-d]pyrimidine-2,4-diamine

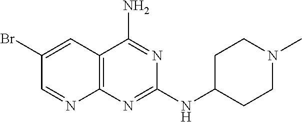 [SMILES: CN1CCC(Nc2nc(N)c3cc(Br)cnc3n2)CC1]

### Route A

### Preparation 45—N-(5-bromo-3-cyanopyridin-2-yl)cyanamide

To a solution of 5-bromo-2-chloronicotinonitrile (1.00 g, 4.60 mmol) in DMSO (10.0 mL) at room temperature was added cyanamide, monosodium salt (0.290 g, 4.60 mmol). The resulting mixture was stirred at 80° C. for 1 h. The reaction was quenched with water at room temperature. The resulting mixture was filtered. The filter cake was purified by Prep-HPLC^{P} to afford the title compound (450 mg, 43.9%). LCMS m/z=225 [M+H]⁺

### Preparation 46—6-bromo-N²-(1-methylpiperidin-4-yl)pyrido[2,3-d]pyrimidine-2,4-diamine

To a solution of N-(5-bromo-3-cyanopyridin-2-yl)cyanamide (300 mg, 1.34 mmol) in dioxane (5.0 mL) at room temperature was added 1-methylpiperidin-4-amine (307 mg, 2.69 mmol). The resulting mixture was stirred at 100° C. for 1.5 h. The resulting mixture was concentrated under vacuum. The residue was purified by Prep-HPLC^{A} to afford the title compound as a white solid (340 mg, 74.1%). ¹H NMR (400 MHz, DMSO-d₆) δ 8.62 (s, 2H), 7.81-7.35 (m, 2H), 7.11-6.51 (m, 1H), 3.87-3.71 (m, 1H), 2.74 (d, J=8.0 Hz, 2H), 2.15 (s, 3H), 1.93 (t, J=12.0 Hz, 2H), 1.84-1.74 (m, 2H), 1.58-1.45 (m, 2H). LCMS^{L} m/z=339 [M+H]⁺.

### Route B

### Preparation 47—6-bromo-2-chloropyrido[2,3-d]pyrimidin-4-amine

A solution of 6-bromo-2,4-dichloropyrido[2,3-d]pyrimidine (200 mg, 0.717 mmol) and NH₃·H₂O (3.0 mL) was stirred was stirred at room temperature for 1 h. The resulting mixture was concentrated under vacuum to afford the title compound (200 mg, crude) as a yellow solid. LCMS m/z=261 [M+H]⁺

### Preparation 48—6-bromo-N²-(1-methylpiperidin-4-yl)pyrido[2,3-d]pyrimidine-2,4-diamine

To a solution of 6-bromo-2-chloropyrido[2,3-d]pyrimidin-4-amine (90.0 mg, 0.347 mmol) and 1-methylpiperidin-4-amine (396 mg, 3.47 mmol) in DMSO (1.0 mL) was added Cs₂CO₃ (339 mg, 1.04 mmol) at room temperature. The resulting mixture was stirred at 100° C. for 3 h. The resulting mixture was purified by Prep-HPLC^{AK} to afford the title compound (14.1 mg, 12.1%) as a yellow oil. ¹H NMR (400 MHz, DMSO-d₆) δ 8.63 (s, 2H), 7.87-7.34 (m, 2H), 7.06-6.79 (m, 1H), 3.86-3.72 (m, 1H), 2.74 (dd, J=11.2, 4.3 Hz, 2H), 2.15 (s, 3H), 2.01-1.87 (m, 2H), 1.85-1.73 (m, 2H), 1.59-1.44 (m, 2H). LCMS^{H} m/z=339 [M+H]⁺

### Example 21: 6-bromo-7-methyl-N-(1-methylpiperidin-4-yl)pyrido[2,3-d]pyrimidine-2,4-diamine

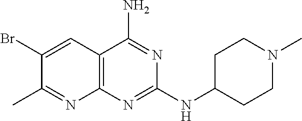 [SMILES: Cc1nc2nc(NC3CCN(C)CC3)nc(N)c2cc1Br]

### Preparation 49—5-bromo-2-(cyanoamino)-6-methylpyridine-3-carbonitrile

To a mixture of 5-bromo-2-chloro-6-methylpyridine-3-carbonitrile (500 mg, 2.16 mmol) in DMSO (5.00 mL) was added cyanamide, monosodium salt (207 mg, 3.24 mmol). The mixture was stirred at 80° C. overnight. The resulting mixture was purified by Prep-HPLC^{AC} to afford the title compound (168 mg, crude) as a brown solid. LCMS m/z=239 [M+H]⁺

### Preparation 50—6-bromo-7-methyl-N²-(1-methylpiperidin-4-yl)pyrido[2,3-d]pyrimidine-2,4-diamine

To a mixture of 5-bromo-2-(cyanoamino)-6-methylpyridine-3-carbonitrile (158 mg, 0.533 mmol) in dioxane (2.50 mL) was added 1-methylpiperidin-4-amine (121 mg, 1.06 mmol). The mixture was stirred at 100° C. for 2 h. The resulting mixture was concentrated under reduced pressure. The crude product was purified by Prep-HPLC^{AD} to afford the title compound as a white solid (21.7 mg, 11.5%). ¹H NMR (400 MHz, DMSO-d₆) δ 8.58 (s, 1H), 7.78-7.14 (br, 2H), 6.68 (s, 1H), 3.84-3.72 (m, 1H), 2.74 (d, J=11.5 Hz, 2H), 2.56 (s, 3H), 2.16 (s, 3H), 1.94 (t, J=11.5 Hz, 2H), 1.80 (d, J=12.3 Hz, 2H), 1.58-1.44 (m, 2H). LCMS^{C} m/z=353 [M+H]⁺

### Example 22: 6-bromo-5,7-dimethyl-N²-(1-methylpiperidin-4-yl)pyrido[2,3-d]pyrimidine-2,4-diamine

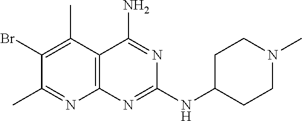 [SMILES: Cc1nc2nc(NC3CCN(C)CC3)nc(N)c2c(C)c1Br]

### Preparation 51—N-(5-bromo-3-cyano-4,6-dimethylpyridin-2-yl)cyanamide

To a solution of 5-bromo-2-chloro-4,6-dimethylnicotinonitrile (2.00 g, 8.14 mmol) and cyanamide, monosodium salt (1.04 g, 16.2 mmol) in DMSO (20.0 mL) was stirred at 80° C. for 2 h. The mixture was allowed to cool down to room temperature and acidified to pH 6 with aq. HCl (1 M). The solid was collected by filtration. The solid was purified by trituration with PE/EA (2:1, 30 mL) to afford the title compound as a light brown solid (1.3 g, 63.6%). LCMS m/z=253 [M+H]⁺

### Preparation 52—6-bromo-5,7-dimethyl-N²-(1-methylpiperidin-4-yl)pyrido[2,3-d]pyrimidine-2,4-diamine

To a solution of N-(5-bromo-3-cyano-4,6-dimethylpyridin-2-yl)cyanamide (100 mg, 0.398 mmol) and 1-methylpiperidin-4-amine (90.8 mg, 0.796 mmol) in dioxane (2.00 mL) was stirred at 100° C. overnight. The mixture was allowed to cool down to room temperature. The resulting mixture was concentrated under reduced pressure. The residue was purified by Prep-HPLC^{AE} to afford the title compound as a white solid (21.9 mg, 15.0%). ¹H NMR (400 MHz, DMSO-d₆) δ 7.00-6.88 (br, 2H), 6.73-6.70 (br, 1H), 3.81-3.71 (m, 1H), 2.76-2.73 (m, 5H), 2.50 (s, 3H), 2.16 (s, 3H), 1.97-1.92 (m, 2H), 1.80-1.77 (m, 2H), 1.56-1.46 (m, 2H). LCMS^{C} m/z=267 [M+H]⁺

### Example 23: 7-chloro-N²-(1-methylpiperidin-4-yl)pyrido[2,3-d]pyrimidine-2,4-diamine

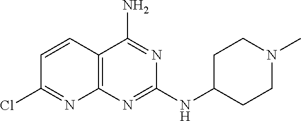 [SMILES: CN1CCC(Nc2nc(N)c3ccc(Cl)nc3n2)CC1]

### Route A

### Preparation 53—2,7-dichloropyrido[2,3-d]pyrimidin-4-amine

A solution of 2,4,7-trichloropyrido[2,3-d]pyrimidine (200 mg, 0.853 mmol) in NH₃·H₂O (3.0 mL) was stirred for 1 h at room temperature. The resulting mixture was concentrated under reduced pressure to afford the title compound as white solid (190 mg, crude). LCMS m/z=217 [M+H]⁺

### Preparation 54—7-chloro-N²-(1-methylpiperidin-4-yl)pyrido[2,3-d]pyrimidine-2,4-diamine

To a stirred solution of 2,7-dichloropyrido[2,3-d]pyrimidin-4-amine (150 mg, 0.698 mmol) and 1-methylpiperidin-4-amine (398 mg, 3.49 mmol) in DMSO (3.00 mL) was added DIEA (180 mg, 1.40 mmol) at room temperature. The resulting mixture was stirred at 60° C. for 1 h. The mixture was allowed to cool down to room temperature. The crude product was purified by Prep-HPLC^{U} to afford 7-chloro-N²-(1-methylpiperidin-4-yl)pyrido[2,3-d]pyrimidine-2,4-diamine as an off-white solid (6.9 mg, 3.34%). ¹H NMR (400 MHz, DMSO-d⁶) δ 8.40-8.30 (m, 1H), 8.00-7.37 (m, 2H), 7.16-6.82 (m, 2H), 3.85-3.72 (m, 1H), 2.80-2.68 (m, 2H), 2.16 (s, 3H), 2.00-1.86 (m, 2H), 1.84-1.73 (m, 2H), 1.58-1.38 (m, 2H). LCMS^{H} m/z=293 [M+H]⁺.

### Route B

### Preparation 55—6-chloro-2-(cyanoamino)pyridine-3-carbonitrile

To a solution of 2,6-dichloropyridine-3-carbonitrile (30.0 g, 173 mmol) in DMSO (240 mL) at room temperature was added cyanamide, monosodium salt (22.2 g, 347 mmol). The resulting mixture was stirred at 60° C. for 1 h. The mixture was allowed to cool down to room temperature. The residue was purified by Prep-HPLC^{B} to afford the title compound as a red solid (10.0 g, 32.3%). LCMS m/z=179 [M+H]⁺

### Preparation 56—7-chloro-N²-(1-methylpiperidin-4-yl)pyrido[2,3-d]pyrimidine-2,4-diamine

To a stirred solution of 6-chloro-2-(cyanoamino)pyridine-3-carbonitrile (10.0 g, 56.0 mmol) in dioxane (150 mL) was added 1-methylpiperidin-4-amine (7.67 g, 67.2 mmol) at room temperature. The resulting mixture was stirred at 100° C. for 1 h. The mixture was allowed to cool down to room temperature and concentrated under reduced pressure. The residue was dissolved in DMF (10.0 mL). The residue was purified by Prep-HPLC^{B} to afford the title compound as a yellow solid (3.00 g, 18.3%). ¹H NMR (400 MHz, DMSO-d6) δ 8.40-8.30 (m, 1H), 8.00-7.37 (m, 2H), 7.16-6.82 (m, 2H), 3.85-3.72 (m, 1H), 2.80-2.68 (m, 2H), 2.16 (s, 3H), 2.00-1.86 (m, 2H), 1.84-1.73 (m, 2H), 1.58-1.38 (m, 2H).

### Example 24: 7-(4-methoxyphenyl)-N²-(1-methylpiperidin-4-yl)pyrido[2,3-d]pyrimidine-2,4-diamine

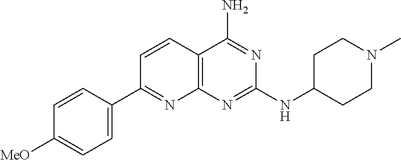 [SMILES: COc1ccc(-c2ccc3c(N)nc(NC4CCN(C)CC4)nc3n2)cc1]

### Preparation 57—N-(3-cyano-6-(4-methoxyphenyl)pyridin-2-yl)cyanamide

To a solution of 6-chloro-2-(cyanoamino)pyridine-3-carbonitrile (150 mg, 0.840 mmol, synthesised as described in Example 23, Preparation 55) and (4-methoxyphenyl)boronic acid (255 mg, 1.68 mmol) in 1,4-dioxane (3.0 mL) and H₂O (0.6 mL) were added Pd(AMPHOS)₂Cl₂ (59.5 mg, 0.084 mmol) and K₃PO₄ (356 mg, 1.68 mmol) at room temperature under nitrogen atmosphere. The resulting mixture was stirred under nitrogen atmosphere at 100° C. for 3 h. The resulting mixture was purified by Prep-HPLC^{B} to afford the title compound as a yellow solid (80.0 mg, 38.1%). LCMS m/z=251 [M+H]⁺

### Preparation 58—7-(4-methoxyphenyl)-N²-(1-methylpiperidin-4-yl)pyrido[2,3-d]pyrimidine-2,4-diamine

To a stirred solution of N-(3-cyano-6-(4-methoxyphenyl) pyridin-2-yl)cyanamide (80.0 mg, 0.320 mmol) in 1,4-dioxane (1.0 mL) at room temperature was added 1-methylpiperidin-4-amine (73.0 mg, 0.640 mmol). The resulting mixture was stirred at 80° C. for 4 h. The resulting mixture was concentrated under vacuum. The residue was purified by Prep-HPLC^{I} to afford the title compound as a white solid (17.0 mg, 14.2%). ¹H NMR (400 MHz, DMSO-d⁶) δ 8.37 (d, J=8.3 Hz, 1H), 8.14 (d, J=8.0 Hz, 2H), 7.56 (d, J=8.0 Hz, 1H), 7.32 (s, 2H), 7.05 (d, J=8.0 Hz, 2H), 6.52 (s, 1H), 3.94-3.76 (m, 4H), 2.75 (d, J=11.2 Hz, 2H), 2.17 (s, 3H), 1.98 (t, J=11.5 Hz, 2H), 1.83 (d, J=12.2 Hz, 2H), 1.60-1.45 (m, 2H); LCMS^{M} m/z: 365 [M+H]⁺.

### Example 25: N²-(1-methylpiperidin-4-yl)-7-(pyridin-3-yl)pyrido[2,3-d]pyrimidine-2,4-diamine

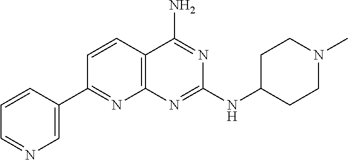 [SMILES: CN1CCC(Nc2nc(N)c3ccc(-c4cccnc4)nc3n2)CC1]

To a stirred solution of 7-chloro-N²-(1-methylpiperidin-4-yl)pyrido[2,3-d]pyrimidine-2,4-diamine (60.0 mg, 0.205 mmol) and pyridin-3-ylboronic acid (50.4 mg, 0.410 mmol) in dioxane (1.5 mL)/H₂O (0.5 mL) were added Pd(AMPhos)₂Cl₂ (14.5 mg, 21.0 μmol) and K₃PO₄ (87.0 mg, 0.410 mmol) at room temperature. The resulting mixture was stirred under nitrogen atmosphere at room temperature for 1 h. The mixture was allowed to cool down to room temperature. The residue was purified by Prep-HPLC^{AF} to afford the title compound (29.4 mg, 42.8%) as a yellow solid. ¹H NMR (400 MHz, DMSO-d₆) δ 9.32 (d, J=2.3 Hz, 1H), 8.67 (dd, J=4.8, 1.6 Hz, 1H), 8.56-8.44 (m, 2H), 7.70 (d, J=8.2 Hz, 1H), 7.54 (dd, J=8.0, 4.7 Hz, 1H), 7.46 (br, 2H), 6.92-6.59 (m, 1H), 3.87 (br, 1H), 2.76 (d, J=11.3 Hz, 2H), 2.17 (s, 3H), 1.96 (d, J=11.7 Hz, 2H), 1.83 (d, J=12.3 Hz, 2H), 1.62-1.47 (m, 2H). LCMS^{C} m/z=336 [M+H]⁺

### Example 26: 7-(1-methyl-1H-pyrazol-4-yl)-N²-(1-methylpiperidin-4-yl)pyrido[2,3-d]pyrimidine-2,4-diamine

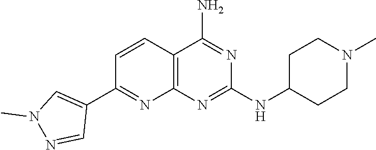

As described for Example 25, using 1-methyl-4-(3,3,4,4-tetramethylborolan-1-yl)-1H-pyrazole and purification by Prep-HPLC^{N} to afford the title compound (14.4 mg, 24.9%) as an off-white solid. ¹H NMR (400 MHz, DMSO-d₆) δ 8.38 (s, 1H), 8.29 (d, J=8.3 Hz, 1H), 8.06 (s, 1H), 7.57-7.04 (m, 3H), 6.53 (br, 1H), 3.90 (s, 3H), 3.83-3.81 (m, 1H), 2.76-2.73 (m, 2H), 2.17 (s, 3H), 1.96 (t, J=11.4 Hz, 2H), 1.81 (d, J=12.2 Hz, 2H), 1.61-1.44 (m, 2H). LCMS^{C} m/z=339 [M+H]⁺

### Example 27: N²-(1-methylpiperidin-4-yl)-7-phenylpyrido[2,3-d]pyrimidine-2,4-diamine

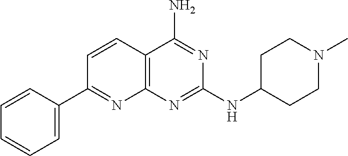 [SMILES: CN1CCC(Nc2nc(N)c3ccc(-c4ccccc4)nc3n2)CC1]

### Route A

As described for Example 25, using phenyl boronic acid and purification by Prep-HPLC^{AH} to afford the title compound as a white solid (22.3 mg, 39.0%). ¹H NMR (300 MHz, DMSO-d₆) δ 8.43 (d, J=9.0 Hz, 1H), 8.20-8.13 (m, 2H), 7.62 (d, J=9.0 Hz, 1H), 7.56-7.35 (m, 5H), 6.70-6.45 (s, 1H), 3.82 (br, 1H), 2.76 (d, J=12.0 Hz, 2H), 2.17 (s, 3H), 1.98 (t, J=12.0 Hz, 2H), 1.83 (d, J=12.0 Hz, 2H), 1.65-1.45 (m, 2H). LCMS^{C} m/z=335 [M+H]⁺.

### Route B

### Preparation 59—7-chloro-1H,3H-pyrido[2,3-d]pyrimidine-2,4-dione

To a solution of 2-amino-6-chloropyridine-3-carboxamide (4.00 g, 23.3 mmol) in toluene (40.0 mL) at room temperature was added (COCl)₂ (3.55 g, 28.0 mmol). The resulting mixture was stirred at 100° C. for 2 h. The mixture was allowed to cool down to room temperature. The precipitated solids were collected by filtration and washed with toluene (3×5 mL). The resulting solid was dried under vacuum to afford the title compound (4.00 g, crude) as a brown solid. LCMS m/z=198 [M+H]⁺.

### Preparation 60—7-phenyl-1H,3H-pyrido[2,3-d]pyrimidine-2,4-dione

To a stirred solution of 7-chloro-1H,3H-pyrido[2,3-d]pyrimidine-2,4-dione (3.00 g, 15.2 mmol) and phenyl boronic acid (2.22 g, 18.2 mmol) in 1,4-dioxane (60.0 mL) and H₂O (20.0 mL) at room temperature were added Pd(AMPHOS)₂Cl₂ (1.08 g, 1.52 mmol) and K₃PO₄ (6.45 g, 30.4 mmol). The resulting mixture was stirred under nitrogen atmosphere at 100° C. for 2 h. The mixture was allowed to cool down to room temperature. The resulting mixture was diluted with H₂O (100 mL) and extracted with CH₂Cl₂ (3×200 mL). The combined organic layers were dried over anhydrous Na₂SO₄. After filtration, the combined filtrate was concentrated under reduced pressure. The residue was purified by trituration with Et₂O (200 mL) to afford the title compound (3.50 g, crude) as a brown solid. LCMS m/z=240 [M+H]⁺.

### Preparation 61—2,4-dichloro-7-phenylpyrido[2,3-d]pyrimidine

A mixture of 7-phenylpyrido[2,3-d]pyrimidine-2,4(1H,3H)-dione (2.80 g, 11.7 mmol) in phosphorus oxychloride (100 mL) was stirred at 110° C. overnight. The resulting mixture was concentrated under reduced pressure. The residue was quenched by the addition of sat. NaHCO₃ (aq.) (100 mL) at 0° C. The resulting mixture was extracted with EtOAc (3×100 mL), dried over anhydrous Na₂SO₄. After filtration, the filtrate was concentrated under reduced pressure. The residue was purified by silica gel column chromatography, eluted with PE/EA (2:1) to afford the title compound (640 mg, 19.9%) as a green solid. LCMS m/z=278 [M+H]⁺

### Preparation 62—2-chloro-7-phenylpyrido[2,3-d]pyrimidin-4-amine

A solution of 2,4-dichloro-7-phenylpyrido[2,3-d]pyrimidine (200 mg, 0.724 mmol) in NH₃—H₂O (3.0 mL) was stirred was stirred at room temperature for 1 h. The resulting mixture was concentrated under vacuum to afford the title compound (230 mg, crude) as a white solid. LCMS m/z=257 [M+H]⁺

### Preparation 63—N²-(1-methylpiperidin-4-yl)-7-phenylpyrido[2,3-d]pyrimidine-2,4-diamine

A solution of 2-chloro-7-phenylpyrido[2,3-d]pyrimidin-4-amine (190 mg, 0.740 mmol) in dioxane (3.0 mL) at room temperature was added 1-methylpiperidin-4-amine (169 mg, 1.48 mmol). The resulting mixture was stirred at 100° C. for 10 h. The resulting mixture was concentrated under reduced pressure. The residue was purified by Prep-HPLC^{AL} to afford the title compound (10.1 mg, 4.03%) as a yellow solid. ¹H NMR (400 MHz, DMSO-d₆) δ 8.43 (d, J=8.3 Hz, 1H), 8.21-8.12 (m, 2H), 7.62 (d, J=8.3 Hz, 1H), 7.56-7.44 (m, 5H), 6.88-6.49 (m, 1H), 3.87 (br, 1H), 2.75 (d, J=10.9 Hz, 2H), 2.17 (s, 3H), 2.00-1.95 (m, 2H), 1.84-1.82 (m, 2H), 1.65-1.45 (m, 2H). LCMS^{H} m/z=335 [M+H]⁺

### Example 28: N²-(1-methylpiperidin-4-yl)-6-(oxetan-3-yloxy)pyrido[2,3-d]pyrimidine-2,4-diamine

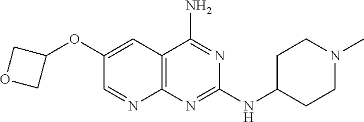 [SMILES: CN1CCC(Nc2nc(N)c3cc(OC4COC4)cnc3n2)CC1]

To a solution of 6-bromo-N²-(1-methylpiperidin-4-yl)pyrido[2,3-d]pyrimidine-2,4-diamine (200 mg, 0.593 mmol) and oxetan-3-ol (87.9 mg, 1.19 mmol) in toluene (4.0 mL) were added Rockphos (41.7 mg, 89.0 μmol), Cs₂CO₃ (290 mg, 0.889 mmol) and bis(chloro(prop-2-en-1-yl)palladium) (10.9 mg, 30.0 μmol) at room temperature. The resulting mixture was stirred under nitrogen atmosphere at 90° C. for 2 h. The resulting mixture was concentrated under reduced pressure. The residue was purified by Prep-HPLC^{AK} to afford the title compound (2.8 mg, 1.43%) as a yellow solid. ¹H NMR (400 MHz, Methanol-d₄) δ 8.48 (s, 1H), 7.66 (s, 1H), 5.35-5.34 (m, 1H), 5.09-5.06 (m, 2H), 4.88-4.71 (m, 2H), 4.18 (s, 1H), 3.49-3.46 (m, 2H), 3.25-3.12 (m, 2H), 2.80 (s, 3H), 2.22-2.20 (m, 2H), 1.97-1.86 (m, 2H). LCMS^{H} m/z=331 [M+H]⁺

### Example 29: N²-(3-(2-oxa-6-azaspiro[3.3]heptan-6-yl)propyl)-5,7-dimethylpyrido[2,3-d]pyrimidine-2,4-diamine

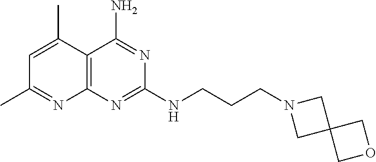 [SMILES: Cc1cc(C)c2c(N)nc(NCCCN3CC4(COC4)C3)nc2n1]

### Preparation 64—N²-(3,3-diethoxypropyl)-5,7-dimethylpyrido[2,3-d]pyrimidine-2,4-diamine

### Route A

To a mixture of 2-(cyanoamino)-4,6-dimethylpyridine-3-carbonitrile (5.00 g, 29.0 mmol) in dioxane (50.0 mL) was added 3,3-diethoxypropan-1-amine (9.40 mL, 58.0 mmol). The mixture was stirred at 100° C. for 1 h. The resulting mixture was concentrated under reduced pressure. The residue was purified by trituration with EA/PE (30.0 mL/60 mL). The solid was collected by filtration and washed with EA/PE (1:2, 3×10 mL). This resulted in the title compound as a brown solid (4.48 g, crude). LCMS m/z=320 [M+H]⁺

### Route B

To a stirred mixture of 2-chloro-5,7-dimethylpyrido[2,3-d]pyrimidin-4-amine (4.30 g, 20.6 mmol, synthesized as described in Example 5, Preparation 13) in 1,4-dioxane (86.0 mL) was added 3,3-diethoxypropan-1-amine (4.55 g, 30.9 mmol, 1.50 equiv) dropwise at room temperature. The resulting mixture was stirred at 100° C. for 5 h. The mixture was allowed to cool down to room temperature and concentrated under reduced pressure. The residue was purified by Prep-HPLC^{B} to afford the title compound (2.33 g, 35.4%) as a yellow solid. LCMS m/z=320 [M+H]⁺

### Preparation 65—3-((4-amino-5,7-dimethylpyrido[2,3-d]pyrimidin-2-yl)amino)propanal

To a mixture of N²-(3,3-diethoxypropyl)-5,7-dimethylpyrido[2,3-d]pyrimidine-2,4-diamine (1.00 g, 3.13 mmol) in DCM (10.0 mL) was added TFA (2.00 mL). The mixture was stirred at room temperature for 1 h. The resulting mixture was concentrated under reduced pressure. The residue was purified by trituration with EA/PE (10.0 mL/5 mL). The crude product was collected by filtration and dried under vacuum to afford the title compound as a white solid (570 mg, crude). LCMS m/z=246 [M+H]⁺

### Preparation 66—N²-(3-(2-oxa-6-azaspiro[3,3]heptan-6-yl)propyl)-5,7-dimethylpyrido[2,3-d]pyrimidine-2,4-diamine

To a solution of 3-((4-amino-5,7-dimethylpyrido[2,3-d]pyrimidin-2-yl)amino)propanal (80.0 mg, 0.326 mmol) in MeOH (2.0 mL) was added Et₃N (131 mg, 1.30 mmol) and 2-oxa-6-azaspiro[3.3]heptane hemioxalate (188 mg, 0.652 mmol). The solution was stirred at room temperature for 1 h and followed by the addition of NaBH₄ (24.6 mg, 0.652 mmol). The resulting mixture was stirred at room temperature for additional 1 h and concentrated under reduced pressure. The crude product was purified by Prep-HPLC^{N} to afford the title compound as a white solid (22.2 mg, 20.7%). ¹H NMR (400 MHz, DMSO-d₆) δ 6.85-6.61 (m, 3H), 6.47 (s, 1H), 4.58 (s, 4H), 3.28-3.21 (m, 2H), 3.21 (s, 4H), 2.63 (s, 3H), 2.37 (s, 3H), 2.33 (t, J=7.0 Hz, 2H), 1.54-1.45 (m, 2H). LCMS^{H} m/z=329 [M+H]⁺

### Example 30: N²-(2-(2-oxa-6-azaspiro[3.3]heptan-6-yl)ethyl)-5,7-dimethylpyrido[2,3-d]pyrimidine-2,4-diamine

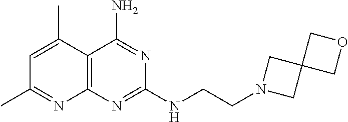 [SMILES: Cc1cc(C)c2c(N)nc(NCCN3CC4(COC4)C3)nc2n1]

As described for Example 29, using 2,2-diethoxyethan-1-amine and final purification by Prep-HPLC^{Y} to afford the title compound (28.4 mg, 26.1%) as a white solid. ¹H NMR (400 MHz, DMSO-d₆) δ 6.74 (s, 2H), 6.69 (s, 1H), 6.30 (s, 1H), 4.58 (s, 4H), 3.25-3.16 (m, 4H), 3.19-3.11 (m, 2H), 2.63 (s, 3H), 2.49-2.44 (m, 2H), 2.37 (s, 3H). LCMS^{H} m/z=315 [M+H]⁺

### Example 31: N³-(1-methylpiperidin-4-yl)-6-phenylpyrimido[4,5-c]isoquinoline-1,3-diamine

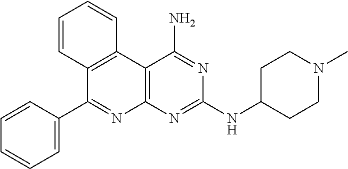 [SMILES: CN1CCC(Nc2nc(N)c3c(n2)nc(-c2ccccc2)c2ccccc23)CC1]

### Preparation 67—1-(3-chloro-1-phenylisoquinolin-4(3H)-ylidene)-N,N-dimethylmethanamine

A solution of POCl₃ (6.72 g, 43.8 mmol) in THF (15 mL) was treated with DMF (3.20 g, 43.7 mmol) for 30 min at 0° C. followed by the addition of 1-phenyl-1,4-dihydroisoquinolin-3(2H)-one (2.47 g, 10.9 mmol). The resulting mixture was stirred for 3 h at room temperature. The reaction was quenched by the addition of NaOH (2 mol/L, 20 mL) at 0° C. The resulting mixture was extracted with EtOAc (3×50 mL). The combined organic layers were washed with brine (3×10 mL), dried over anhydrous Na₂SO₄, filtered, and concentrated under reduced pressure. This resulted in the title compound as a brown oil (5.3 g, crude). LCMS m/z=297 [M+H]⁺

### Preparation 68—3-chloro-1-phenylisoquinoline-4-carbaldehyde

A solution of 1-(3-chloro-1-phenylisoquinolin-4(3H)-ylidene)-N,N-dimethylmethanamine (5.2 g, 17.5 mmol) and KMnO₄ (1.50 mg) in H₂SO₄ (50 mL) was stirred for 1 h at room temperature. The mixture was allowed to cool down to room temperature. The reaction was quenched with water at room temperature. The precipitated solids were collected by filtration and washed with water (3×10 mL). The residue was purified by trituration with EtOAc (20 mL). The resulting mixture was filtered, the filter cake was washed with EtOAc (3×10 mL). The filtrate was concentrated under reduced pressure. This resulted in the title compound (1.5 g, crude) as a brown solid. LCMS m/z=268 [M+H]⁺

### Preparation 69—N-[(3-chloro-1-phenylisoquinolin-4-yl)methylidene]hydroxylamine

A solution of 3-chloro-1-phenylisoquinoline-4-carbaldehyde (1.14 g, 4.26 mmol), Et₃N (764 uL, 5.50 mmol) and hydroxylamine hydrochloride (433 mg, 6.23 mmol) in EtOH (5 mL) was stirred for 2 h at room temperature. The resulting mixture was concentrated under reduced pressure. The resulting mixture was diluted with water (5 mL). The resulting mixture was extracted with EtOAc (3×10 mL). The combined organic layers were washed with brine (3×5 mL), dried over anhydrous Na₂SO₄. After filtration, the filtrate was concentrated under reduced pressure. The residue was purified by Prep-HPLC^{B} to afford the title compound as an orange solid (476 mg, 39.5%). LCMS m/z=283 [M+H]⁺

### Preparation 70—3-chloro-1-phenylisoquinoline-4-carbonitrile

To a stirred solution of N-[(3-chloro-1-phenylisoquinolin-4-yl)methylidene]hydroxylamine (426 mg, 1.51 mmol) in DCM (5.0 mL) was added pyridine (244 uL, 3.01 mmol) dropwise at room temperature. The resulting mixture was stirred for 5 min at 0° C. To the above mixture was added TFAA (838 uL, 6.03 mmol) dropwise at room temperature. The resulting mixture was stirred for additional overnight at room temperature. The resulting mixture was filtered, the filtrate was purified by silica gel column chromatography, eluted with PE/EA (5:1) to afford the title compound as a yellow solid (280 mg, 70.2%). LCMS m/z=265 [M+H]⁺

### Preparation 71—3-(cyanoamino)-1-phenylisoquinoline-4-carbonitrile

To a stirred solution of 3-chloro-1-phenylisoquinoline-4-carbonitrile (270 mg, 1.02 mmol) in DMSO (0.5 mL) was added 2-sodioacetonitrile (129 mg, 2.04 mmol) in portions at room temperature. The resulting mixture was stirred for 1 h at 60° C. The mixture was allowed to cool down to room temperature. The mixture was acidified to pH 3 with 1 mol HCl (aq.) The precipitated solid was collected to afford the title compound (270 mg, crude) as an orange solid. LCMS m/z=271 [M+H]⁺

### Preparation 72—N³-(1-methylpiperidin-4-yl)-6-phenylpyrimido[4,5-c]isoquinoline-1,3-diamine

To a stirred solution of 3-(cyanoamino)-1-phenylisoquinoline-4-carbonitrile (150 mg, 0.555 mmol) in 1,4-dioxane (5.0 mL) was added 1-methylpiperidin-4-amine (126 mg, 1.11 mmol) dropwise at room temperature. The resulting mixture was stirred for 7 h at 100° C. The resulting mixture was concentrated under reduced pressure. The residue was dissolved in DMSO (5.0 mL). The mixture was acidified to pH 5 with 1M HCl (aq.). Then, the mixture was purified by Prep-HPLC^{J} to afford the title compound as a yellow solid (12.1 mg, 5.6%). ¹H NMR (400 MHz, Methanol-d₄) δ 8.65 (d, J=8.6 Hz, 1H), 8.18 (d, J=8.4 Hz, 1H), 7.99 (t, J=8.1 Hz, 1H), 7.80-7.73 (m, 2H), 7.70-7.58 (m, 4H), 4.33 (s, 1H), 3.71-3.46 (m, 2H), 3.29-3.08 (m, 2H), 2.93 (s, 3H), 2.35 (br., 2H), 2.03 (br. s, 2H). LCMS^{H} m/z=385 [M+H]⁺

### Example 32: N²-(1-methylpiperidin-4-yl)-7-phenoxypyrido[2,3-d]pyrimidine-2,4-diamine

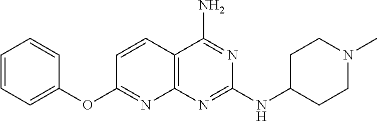 [SMILES: CN1CCC(Nc2nc(N)c3ccc(Oc4ccccc4)nc3n2)CC1]

To a stirred solution of 7-chloro-N²-(1-methylpiperidin-4-yl)pyrido[2,3-d]pyrimidine-2,4-diamine (50.0 mg, 0.171 mmol) and phenol (32.2 mg, 0.342 mmol) in DMSO (1.0 mL) was added t-BuOK (38.3 mg, 0.342 mmol) at room temperature. The resulting mixture was stirred at 100° C. for 2 h. The mixture was allowed to cool down to room temperature. The residue was purified by Prep-HPLC® to afford the title compound (22.5 mg, 37.6%) as a yellow solid. ¹H NMR (400 MHz, DMSO-d₆) δ 8.36 (d, J=8.6 Hz, 1H), 7.44 (t, J=7.7 Hz, 3H), 7.31-7.10 (m, 4H), 6.74-6.39 (m, 2H), 3.81-3.64 (m, 1H), 2.68 (d, J=11.0 Hz, 2H), 2.13 (s, 3H), 1.91 (t, J=11.4 Hz, 2H), 1.81-1.63 (m, 2H), 1.47-1.42 (m, 2H). LCMS^{H} m/z=351 [M+H]⁺

### Example 33: 7-cyclobutoxy-N²-(1-methylpiperidin-4-yl)pyrido[2,3-d]pyrimidine-2,4-diamine

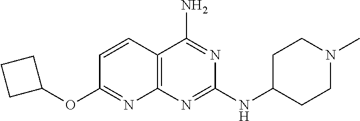 [SMILES: CN1CCC(Nc2nc(N)c3ccc(OC4CCC4)nc3n2)CC1]

To a solution of 7-chloro-N²-(1-methylpiperidin-4-yl)pyrido[2,3-d]pyrimidine-2,4-diamine (50.0 mg, 0.171 mmol) and cyclobutanol (24.6 mg, 0.342 mmol), t-BuOK (38.3 mg, 0.342 mmol) in DMSO (2.00 mL) was stirred at 100° C. overnight. The mixture was allowed to cool down to room temperature. The residue was purified by Prep-HPLC^{B} to afford the title compound as a white solid (4.90 mg, 8.74%). ¹H NMR (400 MHz, DMSO-d⁶) δ 8.21-8.19 (m, 1H), 7.13 (br, 2H), 6.54 (br, 1H), 6.41-6.38 (d, J=12.0, 1H), 5.16 (s, 1H), 3.86 (s, 1H), 2.89-2.87 (m, 2H), 2.43-2.36 (m, 2H), 2.29-2.27 (m, 5H), 2.09-1.99 (m, 2H), 1.91-1.74 (m, 3H), 1.68-1.55 (m, 3H). LCMS m/z=329 [M+H]⁺

### Example 34: N⁷-methyl-N2-(1-methylpiperidin-4-yl)pyrido[2,3-d]pyrimidine-2,4,7-triamine

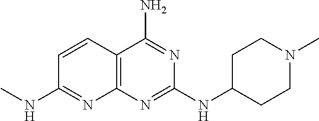

To a stirred solution of 7-chloro-N²-(1-methylpiperidin-4-yl)pyrido[2,3-d]pyrimidine-2,4-diamine (30.0 mg, 0.102 mmol) and methanamine hydrochloride (13.8 mg, 0.204 mmol) in DMSO (0.5 mL) was added Cs₂CO₃ (66.8 mg, 0.204 mmol) in portions at room temperature. The resulting mixture was stirred at 100° C. overnight. The mixture was allowed to cool down to room temperature. The resulting mixture was filtered. The combined filtrate was purified by Prep-HPLC^{B} to afford the title compound (3.40 g, 9.24%) as a light-yellow oil. ¹H NMR (400 MHz, Methanol-d4) 7.86-7.77 (m, 2H), 6.36 (d, J=9.0 Hz, 1H), 4.04-3.94 (m, 1H), 3.05-3.02 (m, 2H), 2.88 (s, 3H), 2.58-2.48 (m, 2H), 2.45 (s, 3H), 2.04-2.02 (m, 2H), 1.73-1.60 (m, 2H). LCMS^{G} m/z=288 [M+H]⁺

### Example 35: N²-(1-methylpiperidin-4-yl)-N⁷-phenylpyrido[2,3-d]pyrimidine-2,4,7-triamine

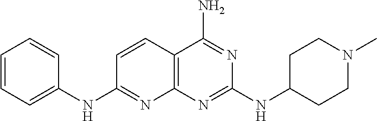 [SMILES: CN1CCC(Nc2nc(N)c3ccc(Nc4ccccc4)nc3n2)CC1]

To a stirred mixture of 7-chloro-N²-(1-methylpiperidin-4-yl)pyrido[2,3-d]pyrimidine-2,4-diamine (100 mg, 0.342 mmol) and aniline (63.6 mg, 0.684 mmol) in 1,4-dioxane (2.0 mL) were added NaOTMS (76.6 mg, 0.684 mmol), EPhos Pd G4 (31.4 mg, 34.0 μmol) and EPhos (18.3 mg, 34.0 μmol) at room temperature. The resulting mixture was stirred under nitrogen atmosphere at 80° C. for 1 h. The resulted mixture was purified Prep-HPLC^{AM} to afford the title compound (8.8 mg, 7.37%) as an off-white solid. ¹H NMR (400 MHz, DMSO-d₆) δ 9.27 (s, 1H), 8.04 (d, J=8.7 Hz, 1H), 7.95-7.75 (m, 2H), 7.29 (t, J=7.7 Hz, 2H), 7.03 (s, 2H), 6.94 (t, J=7.3 Hz, 2H), 6.47 (d, J=8.7 Hz, 1H), 3.94-3.71 (m, 1H), 2.73 (d, J=11.4 Hz, 2H), 2.16 (s, 3H), 1.96 (t, J=11.5 Hz, 2H), 1.85-1.74 (m, 2H), 1.49 (q, J=11.5 Hz, 2H). LCMS^{H} m/z=350 [M+H]⁺

### Example 36: N⁷,N⁷-dimethyl-N²-(1-methylpiperidin-4-yl)pyrido[2,3-d]pyrimidine-2,4,7-triamine

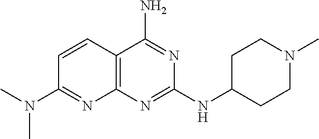

To a stirred solution of 7-chloro-N²-(1-methylpiperidin-4-yl)pyrido[2,3-d]pyrimidine-2,4-diamine (50.0 mg, 0.171 mmol) and dimethylamine hydrochloride (20.6 mg, 0.257 mmol) in DMSO (1.0 mL) at room temperature was added Cs₂CO₃ (111 mg, 0.342 mmol). The resulting mixture was stirred at 100° C. overnight. The mixture was allowed to cool down to room temperature. The crude product was purified by Prep-HPLC^{AL} to afford the title compound as a yellow solid (18.1 mg, 33.1%). ¹H NMR (400 MHz, DMSO-d₆) δ 8.01 (dd, J=9.0, 2.4 Hz, 1H), 6.85 (s, 2H), 6.43 (d, J=8.9 Hz, 1H), 6.13 (s, 1H), 3.86-3.67 (m, 1H), 3.08 (s, 6H), 2.78-2.66 (m, 2H), 2.15 (s, 3H), 1.95 (t, J=10.8 Hz, 2H), 1.78 (d, J=12.8 Hz, 2H), 1.59-1.36 (m, 2H). LCMS^{H} m/z=302 [M+H]⁺

### Example 37: N²-(2-(isoindolin-2-yl)ethyl)-5,7-dimethylpyrido[2,3-d]pyrimidine-2,4-diamine

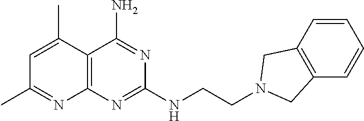 [SMILES: Cc1cc(C)c2c(N)nc(NCCN3Cc4ccccc4C3)nc2n1]

As described for Example 29, using isoindoline and purification by Prep-HPLC^{O} to afford the title compound as white solid (5.6 mg, 5.78%). ¹H NMR (400 MHz, DMSO-d⁶) δ 7.31-7.07 (m, 4H), 6.77-6.45 (m, 4H), 3.91 (s, 4H), 3.49-3.46 (m, 2H), 2.86 (t, J=6.7 Hz, 2H), 2.64 (s, 3H), 2.38 (s, 3H). LCMS^{H} m/z=335 [M+H]⁺

### Example 38: 7-isopropoxy-N²-(1-methylpiperidin-4-yl)pyrido[2,3-d]pyrimidine-2,4-diamine

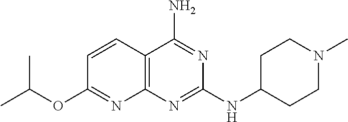 [SMILES: CC(C)Oc1ccc2c(N)nc(NC3CCN(C)CC3)nc2n1]

To a solution of 7-chloro-N²-(1-methylpiperidin-4-yl)pyrido[2,3-d]pyrimidine-2,4-diamine (50.0 mg, 0.171 mmol) and isopropyl alcohol (1.00 mL, 13 mmol), Cs₂CO₃ (111 mg, 0.342 mmol) in DMSO (1.00 mL) was stirred at 100° C. overnight. The mixture was allowed to cool down to room temperature. The crude product was purified by Prep-HPLC^{AL} to afford the title compound as a white solid (6.50 mg, 12.0%). ¹H NMR (400 MHz, DMSO-d⁶) δ 8.18-8.16 (d, J=8.00, 1H), 7.09 (m, 2H), 6.36-6.33 (m, 2H), 5.78 (br m, 1H), 3.79 (s, 1H), 3.74-3.71 (d, J=12.0, 2H), 2.16 (s, 3H), 1.98-1.93 (m, 2H), 1.79-1.77 (m, 2H), 1.54-1.46 (m, 2H), 1.33 (d, J=16.0, 6H). LCMS^{C} m/z: 317 [M+H]⁺

### Example 39: N⁴-methyl-N²-(1-methylpiperidin-4-yl)pyrido[2,3-d]pyrimidine-2,4-diamine

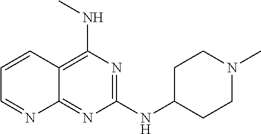 [SMILES: CNc1nc(NC2CCN(C)CC2)nc2ncccc12]

### Preparation 73—2-chloro-N-methylpyrido[2,3-d]pyrimidin-4-amine

To a stirred solution of 2,4-dichloropyrido[2,3-d]pyrimidine (500 mg, 2.50 mmol) and methanamine hydrochloride (338 mg, 5.00 mmol) in DMSO (5.0 mL) at room temperature was added Cs₂CO₃ (1.63 g, 5.00 mmol). The resulting mixture was stirred at 60° C. for 1 h. The resulting mixture was diluted with water (5 mL). The precipitated solids were collected by filtration and washed with water. This resulted in the title compound as a white solid (330 mg, crude). LCMS m/z=195 [M+H]⁺

### Preparation 74—N⁴-methyl-N²-(1-methylpiperidin-4-yl)pyrido[2,3-d]pyrimidine-2,4-diamine

To a stirred solution of 2-chloro-N-methylpyrido[2,3-d]pyrimidin-4-amine (150 mg, 0.771 mmol) in dioxane (3.0 mL) at room temperature was added 1-methylpiperidin-4-amine (176 mg, 1.54 mmol). The resulting mixture was stirred at 100° C. overnight. The resulting mixture was concentrated under reduced pressure. The residue was purified by Prep-H PLC^{AK} to afford the title compound as a white solid (4.0 mg, 1.71%). ¹H NMR (400 MHz, DMSO-d₆) δ 8.57 (dd, J=4.5, 1.9 Hz, 1H), 8.27 (dd, J=8.0, 2.0 Hz, 1H), 8.02 (s, 1H), 7.13-6.91 (m, 1H), 6.69 (s, 1H), 3.93-3.72 (m, 1H), 2.94 (d, J=4.4 Hz, 3H), 2.83-2.67 (m, 2H), 2.16 (s, 3H), 1.95 (td, J=11.8, 2.5 Hz, 2H), 1.83 (d, J=12.3 Hz, 2H), 1.62-1.46 (m, 2H). LCMS^{G} m/z=273 [M+H]⁺

### Example 40: N⁴,N⁴-dimethyl-N²-(1-methylpiperidin-4-yl)pyrido[2,3-d]pyrimidine-2,4-diamine

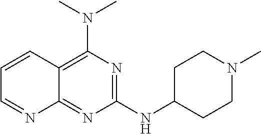 [SMILES: CN1CCC(Nc2nc(N(C)C)c3cccnc3n2)CC1]

### Preparation 75—2-chloro-N,N-dimethylpyrido[2,3-d]pyrimidin-4-amine

To a stirred solution of 2,4-dichloropyrido[2,3-d]pyrimidine (300 mg, 1.50 mmol) and dimethylamine hydrochloride (245 mg, 3.00 mmol) in DMSO (5.0 mL) was added Cs₂CO₃ (977 mg, 3.00 mmol) at room temperature. The resulting mixture was stirred at 60° C. for 1 h. The resulting mixture was diluted with water (5.0 mL). The residue was purified by Prep-HPLC^{B} to afford the title compound (180 mg, 57.5%). LCMS m/z=209 [M+H]⁺

### Preparation 76—N⁴,N⁴-dimethyl-N²-(1-methylpiperidin-4-yl)pyrido[2,3-d]pyrimidine-2,4-diamine

To a stirred solution of 2-chloro-N,N-dimethylpyrido[2,3-d]pyrimidin-4-amine (80.0 mg, 0.383 mmol) in dioxane (2.0 mL) at room temperature was added 1-methylpiperidin-4-amine (87.6 mg, 0.766 mmol). The resulting mixture was stirred at 100° C. overnight. The mixture was allowed to cool down to room temperature. The crude product was purified by Prep-HPLC^{AK} to afford the title compound as an off-white solid (31.9 mg, 26.9%). ¹H NMR (400 MHz, DMSO-d⁶) δ 8.62 (s, 1H), 8.33 (s, 1H), 7.06 (s, 2H), 4.07 (s, 1H), 3.42-3.20 (m, 8H), 3.14-2.99 (m, 2H), 2.70 (s, 3H), 2.19-2.03 (m, 2H), 1.92-1.72 (m, 2H). LCMS^{Q} m/z=287 [M+H]⁺

### Example 41: N⁷-(2,4-dimethoxybenzyl)-N2-(1-methylpiperidin-4-yl)pyrido[2,3-d]pyrimidine-2,4,7-triamine

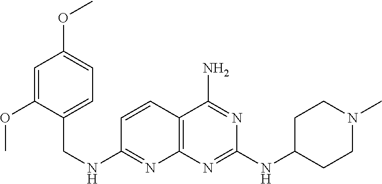 [SMILES: COc1ccc(CNc2ccc3c(N)nc(NC4CCN(C)CC4)nc3n2)c(OC)c1]

To a stirred solution of 7-chloro-N²-(1-methylpiperidin-4-yl)pyrido[2,3-d]pyrimidine-2,4-diamine (300 mg, 1.03 mmol) and (2,4-dimethoxyphenyl)methanamine (514 mg, 3.08 mmol) in DMSO (8.0 mL) at room temperature was added Cs₂CO₃ (1.02 g, 3.08 mmol). The resulting mixture was stirred at 120° C. for 5 h. The mixture was allowed to cool down to room temperature. The resulting mixture was diluted with water (20 mL). The resulting mixture was extracted with EtOAc (3×30 mL). The combined organic layers were washed with brine (2×20 mL), dried over anhydrous Na₂SO₄. After filtration, the filtrate was concentrated under reduced pressure. The residue was purified by Prep-HPLC^{B} to afford titled product (78 mg, 17.90%) as a green solid. ¹H NMR (400 MHz, DMSO-d₆) δ 7.86 (d, J=8.8 Hz, 1H), 7.24-7.09 (m, 2H), 6.83 (s, 2H), 6.56 (d, J=2.4 Hz, 1H), 6.46 (dd, J=8.3, 2.4 Hz, 1H), 6.26 (d, J=8.7 Hz, 1H), 6.10 (s, 1H), 4.42 (d, J=5.6 Hz, 2H), 3.82-3.73 (m, 7H), 2.76-2.67 (m, 2H), 2.15 (s, 3H), 2.00-1.90 (m, 2H), 1.81-1.73 (m, 2H), 1.52-1.41 (m, 2H). LCMS^{H} m/z=424 [M+H]⁺

### Example 42: N²-(1-methylpiperidin-4-yl)-6-(prop-1-yn-1-yl)pyrido[2,3-d]pyrimidine-2,4-diamine

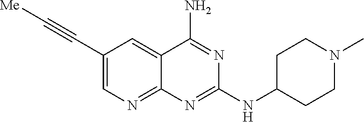 [SMILES: CC#Cc1cnc2nc(NC3CCN(C)CC3)nc(N)c2c1]

To a stirred mixture of 6-bromo-N²-(1-methylpiperidin-4-yl)pyrido[2,3-d]pyrimidine-2,4-diamine (40.0 mg, 0.119 mmol) and tributyl(prop-1-yn-1-yl)stannane (39.0 mg, 0.119 mmol) in toluene (0.5 mL) at room temperature was added Pd(PPh₃)₄ (6.85 mg, 0.006 mmol) under nitrogen atmosphere. The resulting mixture was stirred at 100° C. overnight. The resulting mixture was concentrated under reduced pressure. The residue was purified by Prep-HPLC^{B}. The crude product was further purified by Prep-HPLC^{AT} to afford the title compound as an off-white solid (7.2 mg, 20.3%). ¹H NMR (400 MHz, DMSO-d⁶) δ 8.56 (d, J=2.3 Hz, 1H), 8.43 (s, 1H), 7.79-7.29 (br, 2H), 7.71-6.68 (br, 1H), 3.91-3.71 (m, 1H), 2.81 (d, J=11.1 Hz, 2H), 2.22 (s, 3H), 2.15-1.94 (m, 5H), 1.87-1.74 (m, 2H), 1.61-1.48 (m, 2H). LCMS^{H} m/z=297 [M+H]⁺

### Example 43: N²-(1-methylpiperidin-4-yl)-1,8-naphthyridine-2,4-diamine

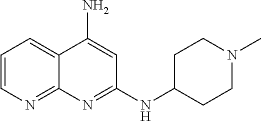 [SMILES: CN1CCC(Nc2cc(N)c3cccnc3n2)CC1]

### Preparation 77—4-chloro-N-(1-methylpiperidin-4-yl)-1,8-naphthyridin-2-amine and 2-chloro-N-(1-methylpiperidin-4-yl)-1,8-naphthyridin-4-amine

A solution of 2,4-dichloro-1,8-naphthyridine (2.00 g, 10.0 mmol), 1-methylpiperidin-4-amine (13.3 mL, 12.1 mmol) and DIEA (3.50 mL, 20.1 mmol) in DMSO (10.0 mL) was stirred at 40° C. for 1 h. The resulting mixture was purified by Prep-HPLC^{B} to afford 4-chloro-N-(1-methylpiperidin-4-yl)-1,8-naphthyridin-2-amine (1.62 g, 58.7%) and 2-chloro-N-(1-methylpiperidin-4-yl)-1,8-naphthyridin-4-amine (1.16 g, 42.0%) as a yellow solid. LCMS m/z=277 [M+H]⁺

### Preparation 78—4-((diphenylmethylene)amino)-N-(1-methylpiperidin-4-yl)-1,8-naphthyridin-2-amine

To a stirring solution of 4-chloro-N-(1-methylpiperidin-4-yl)-1,8-naphthyridin-2-amine (1.00 g, 3.61 mmol) and diphenylmethanimine (727 μL, 4.33 mmol) in 1,4-dioxane (10.0 mL) were added Pd(OAc)₂ (81.1 mg, 0.361 mmol), XantPhos (209 mg, 0.361 mmol) and Cs₂CO₃ (2.35 g, 7.23 mmol). The mixture was stirred under nitrogen atmosphere at 100° C. for 1 h. The resulting mixture was filtered through a short pad of Celite. The pad was washed with CH₂Cl₂ (3×20 mL). The combined filtrate was concentrated under reduced pressure. The residue was purified by silica gel column chromatography, eluted with CH₂Cl₂/MeOH (10:1) to afford the title compound (1.1 g, 72.4%) as a yellow solid. ¹H NMR (400 MHz, DMSO-d₆) δ 8.53 (dd, J=4.4, 1.9 Hz, 1H), 8.28 (dd, J=8.0, 2.0 Hz, 1H), 7.02 (dd, J=8.0, 4.4 Hz, 1H), 6.80 (d, J=7.7 Hz, 1H), 6.49 (s, 2H), 5.86 (s, 1H), 3.90 (s, 1H), 2.90-2.75 (m, 2H), 2.26 (s, 3H), 2.22-2.02 (m, 2H), 1.92 (m, 2H), 1.58-1.43 (m, 2H). LCMS^{H} m/z=258 [M+H]⁺.

### Example 44: 5-methyl-N2-(quinuclidin-4-yl)-7,8-dihydro-6H-cyclopenta[5,6]pyrido[2,3-d]pyrimidine-2,4-diamine

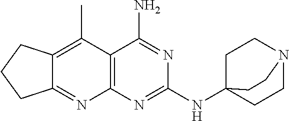 [SMILES: Cc1c2c(nc3nc(NC45CCN(CC4)CC5)nc(N)c13)CCC2]

### Preparation 79—1-azabicyclo[2.2.2]octan-4-amine hydrochloride

To a solution of 1-aza bicyclo[2.2.2]octane-4-carboxylic acid hydrochloride (200 mg, 1.04 mmol) in toluene (2 mL) was added Et₃N (211 mg, 2.09 mmol) and DPPA (0.250 mL, 1.15 mmol) under nitrogen atmosphere at room temperature. The mixture was stirred under nitrogen atmosphere at room temperature for 2 h. The resulting mixture was heated to 100° C. and stirred for additional 3 h. The mixture was allowed to cool down to room temperature and quenched by the addition of conc. HCl (6.00 mL) dropwise at 0° C. The mixture was extracted with EtOAc (3×10 mL). The combined organic phases were concentrated under reduced pressure. Then residue was purified by Prep-HPLC^{L} to afford the title compound as a white semi-solid (500 mg, crude). LCMS m/z=125 [M−H]⁻

### Preparation 80—5-methyl-N²-(quinuclidin-4-yl)-7,8-dihydro-6H-cyclopenta[5,6]pyrido[2,3-d]pyrimidine-2,4-diamine

To a solution of N-(3-cyano-4-methyl-6,7-dihydro-5H-cyclopenta[b]pyridin-2-yl)cyanamide (40.0 mg, 0.202 mmol) and 1-azabicyclo[2.2.2]octan-4-amine hydrochloride (197 mg, 1.21 mmol) in EtOH (1.0 mL) was added Et₃N (40.8 mg, 0.404 mmol). The reaction mixture was stirred at 100° C. overnight. The mixture was allowed to cool down to room temperature and concentrated under reduced pressure. The crude product (150 mg) was purified by Prep-HPLC^{M} to afford the title compound as a white solid (8.1 mg, 12.4%). ¹H NMR (400 MHz, DMSO-d₆) δ 6.60 (s, 2H), 5.77 (s, 1H), 2.90-2.80 (m, 10H), 2.58 (s, 3H), 2.08-1.98 (m, 2H), 1.94 (t, J=7.8 Hz, 6H). LCMS^{L} m/z=325 [M+H]⁺

### Example 45: N³-(1-methylpiperidin-4-yl)-6-phenyl-8,9-dihydro-7H-cyclopenta[4,5]pyrido[2,3-d]pyrimidine-1,3-diamine

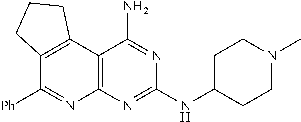 [SMILES: CN1CCC(Nc2nc(N)c3c4c(c(-c5ccccc5)nc3n2)CCC4)CC1]

### Preparation 81—1,3-dihydroxy-6,7-dihydro-5H-cyclopenta[c]pyridine-4-carbonitrile

To a stirred solution of ethyl 2-oxocyclopentane-1-carboxylate (5.00 g, 32.0 mmol) in MeOH (25.0 mL) were added cyanoacetamide (2.83 g, 33.6 mmol) and KOH (1.89 g, 33.6 mmol). The resulting mixture was stirred at 70° C. for 1 h. The mixture was allowed to cool down to room temperature. The mixture was acidified to pH 1 with aq. HCl (1.0 M). The precipitated solids were collected by filtration and washed with water (3×50 mL). The solid was dried under vacuum to afford the title compound (1.68 g crude) as a white solid. LCMS m/z=177 [M+H]⁺.

### Preparation 82—4-cyano-6,7-dihydro-5H-cyclopenta[c]pyridine-1,3-diyl bis(trifluoromethanesulfonate

To a mixture of 1,3-dihydroxy-6,7-dihydro-5H-cyclopenta[c]pyridine-4-carbonitrile (600 mg, 3.40 mmol) and DIEA (1.32 g, 10.2 mmol) in DCM (10.0 mL) were added Tf₂O (2.88 g, 10.2 mmol) dropwise at 0° C. The resulting mixture was stirred at room temperature for 1 h and quenched by the addition of H₂O (30 mL). The mixture was extracted with EtOAc (3×30 mL). The combined organic layers were washed with brine (30 mL), dried over anhydrous Na₂SO₄, filtered and concentrated under reduced pressure. The residue was purified by silica gel column chromatography, eluted with PE/EA (5:1) to afford the title compound as an off-white liquid (1.30 g, crude). LCMS m/z=439 [M−H]⁻

### Preparation 83—4-cyano-1-phenyl-6,7-dihydro-5H-cyclopenta[c]pyridin-3-yl trifluoromethanesulfonate

To a mixture of 4-cyano-6,7-dihydro-5H-cyclopenta[c]pyridine-1,3-diyl bis(trifluoromethanesulfonate) (1.20 g, 2.72 mmol) and phenyl boronic acid (270 mg, 2.18 mmol) in dioxane (12.0 mL) and H₂O (4.0 mL) were added Pd(dppf)Cl₂·CH₂Cl₂ (190 mg, 0.273 mmol) and K₃PO₄ (1.16 g, 5.45 mmol). The resulting mixture was stirred under nitrogen atmosphere at room temperature for 1 h. The resulting mixture was diluted with in H₂O (30 mL) and extracted with EtOAc (3×30 mL). The combined organic layers were washed with brine (30 mL), dried over anhydrous Na₂SO₄, filtered, and concentrated under reduced pressure. The residue was purified by silica gel column chromatography, eluted with PE/EA (5:1) to afford the title compound as a white solid (310 mg, 30.8%). LCMS m/z=369 [M+H]⁺

### Preparation 84—N-(4-cyano-1-phenyl-6,7-dihydro-5H-cyclopenta[c]pyridin-3-yl)cyanamide

To a mixture of 4-cyano-1-phenyl-6,7-dihydro-5H-cyclopenta[c]pyridin-3-yl trifluoromethanesulfonate (300 mg, 0.814 mmol) and cyanamide (68.4 mg, 1.62 mmol) in 1,4-dioxane (5.0 mL) were added Brettphos Pd G3 (73.8 mg, 0.081 mmol), Brettphos (43.7 mg, 0.081 mmol) and Cs₂CO₃ (530 mg, 1.62 mmol) at room temperature. The resulting mixture was stirred under nitrogen atmosphere at 100° C. for 1 h. The mixture was allowed to cool down to room temperature. The resulting mixture was filtered through a short pad of Celite. The pad was washed with MeOH (20 mL×3). The combined filtrate was concentrated under reduced pressure. The residue was purified by Prep-HPLC^{C} to afford the title compound (150 mg, 70.7%) as a yellow solid. LCMS m/z=261 [M+H]⁺

### Preparation 85—N³-(1-methylpiperidin-4-yl)-6-phenyl-8,9-dihydro-7H-cyclopenta[4,5]pyrido[2,3-d]pyrimidine-1,3-diamine

To a mixture of N-(4-cyano-1-phenyl-6,7-dihydro-5H-cyclopenta[c]pyridin-3-yl)cyanamide (60.0 mg, 0.231 mmol) and 1-methylpiperidin-4-amine (52.6 mg, 0.462 mmol) in 1,4-dioxane (3.0 mL) was stirred at 60° C. overnight. The resulting mixture was allowed to cool down to room temperature and concentrated under reduced pressure. The crude product was purified by Prep-HPLC^{AB} to afford the title compound as a light-yellow solid (11.6 mg, 13.4%). ¹H NMR (400 MHz, DMSO-d⁶) δ 7.79-7.77 (m, 2H), 7.55-7.40 (m, 3H), 6.71 (br, 2H), 6.38 (br, 1H), 3.89-3.75 (m, 1H), 3.45-3.36 (m, 2H), 3.00 (t, J=8.0 Hz, 2H), 2.74 (d, J=12.0 Hz, 2H), 2.16 (s, 3H), 2.13-2.03 (m, 2H), 1.96 (t, J=12.0 Hz, 2H), 1.86-1.76 (m, 2H), 1.60-1.45 (m, 2H). LCMS^{H} m/z: 375 [M+H]⁺

### Example 46: 6-methyl-N³-(3-morpholinopropyl)-8,9-dihydro-7H-cyclopenta[4,5]pyrido[2,3-d]pyrimidine-1,3-diamine

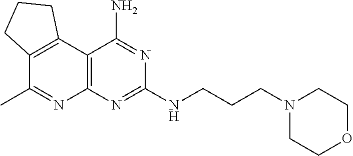 [SMILES: Cc1nc2nc(NCCCN3CCOCC3)nc(N)c2c2c1CCC2]

A solution of N-(4-cyano-1-methyl-6,7-dihydro-5H-cyclopenta[c]pyridin-3-yl)cyanamide (60.0 mg, 0.30 mmol) and 4-morpholinepropanamine (43.7 μL, 0.30 mmol) in EtOH (1.20 mL) was stirred at 80° C. overnight. The mixture was allowed to cool down to room temperature and concentrated under reduced pressure. The residue was purified by Prep-HPLC^{V} to afford the title compound as white solid (33.0 mg, 31.8%). ¹H NMR (400 MHz, DMSO-d₆) δ 6.58-6.54 (m, 3H), 3.58-3.56 (m, 4H), 3.31-3.28 (m, 4H), 2.83-2.79 (m, 2H), 2.51-2.31 (m, 9H), 2.15-2.09 (m, 2H), 1.72-1.65 (m, 2H). LCMS^{O} m/z=343 [M+H]⁺

### Example 47: 5-methyl-N2-(3-morpholinopropyl)-7,8-dihydro-6H-cyclopenta[5,6]pyrido[2,3-d]pyrimidine-2,4-diamine

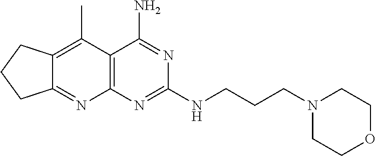 [SMILES: Cc1c2c(nc3nc(NCCCN4CCOCC4)nc(N)c13)CCC2]

To a solution of N-(3-cyano-4-methyl-6,7-dihydro-5H-cyclopenta[b]pyridin-2-yl)cyanamide (60.0 mg, 0.303 mmol) in EtOH (1.00 mL) was added 4-morpholinepropanamine (43.7 uL, 0.303 mmol) at room temperature. The resulting mixture was stirred overnight at 80° C. The mixture was allowed to cool down to room temperature. The crude product was purified by Prep-HPLC^{N} to afford the title compound as white solid (17.1 mg, 16.5%). ¹H NMR (400 MHz, DMSO-d₆) δ 6.84-6.32 (br, 3H), 3.57 (t, J=4.7 Hz, 4H), 3.30 (t, J=8.0 Hz, 2H), 2.88-2.82 (m, 4H), 2.57 (s, 3H), 2.33-2.30 (m, 6H), 2.02-2.01 (m, 2H), 1.68-1.64 (m, 2H). LCMS^{H} m/z=343 [M+H]⁺

### Example 48: 6-chloro-5-methyl-N2-(1-methylpiperidin-4-yl)-7-phenylpyrido[2,3-d]pyrimidine-2,4-diamine

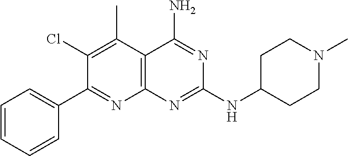 [SMILES: Cc1c(Cl)c(-c2ccccc2)nc2nc(NC3CCN(C)CC3)nc(N)c12]

### Preparation 86—2-hydroxy-6-methyl-4-phenylpyridine-3-carbonitrile

To a solution of 3-oxo-3-phenylpropanal (5.00 g, 30.8 mmol) in EtOH (50.0 mL) was added cyanoacetamide (2.59 g, 30.8 mmol) and 1,4-diazabicyclo[2.2.2]octane (34.6 g, 308 mmol) dropwise at room temperature. The resulting mixture was stirred for 6 h at 80° C. The mixture was allowed to cool down to room temperature. The resulting mixture was stirred overnight at room temperature. The precipitated solids were collected by filtration and washed with MeCN (3 mL) to afford the title compound as white solid (3.20 g, crude). LCMS m/z=211 [M+H]⁺

### Preparation 87—5-chloro-2-hydroxy-6-methyl-4-phenylpyridine-3-carbonitrile

To a solution of 2-hydroxy-6-methyl-4-phenylpyridine-3-carbonitrile (1.00 g, 4.76 mmol) in THF (5.00 mL) and MeOH (5.00 mL) was added NCS (0.950 g, 7.14 mmol) at room temperature. The resulting mixture was stirred for 1 h at 40° C. The mixture was allowed to cool down to room temperature. The precipitated solids were collected by filtration and washed with PE (10 mL). The solid was purified by silica gel column chromatography, eluted with CH₂Cl₂ to afford the title compound as light-yellow solid (780 mg, 67.0%). LCMS m/z=245 [M+H]⁺

### Preparation 88—5-chloro-3-cyano-6-methyl-4-phenylpyridin-2-yl trifluoromethanesulfonate

To a solution of 5-chloro-2-hydroxy-6-methyl-4-phenylpyridine-3-carbonitrile (770 mg, 3.15 mmol) in DCM (7.00 mL) was added Et₃N (0.870 mL, 6.29 mmol) and Tf₂O (1.06 mL, 6.29 mmol) dropwise at 0° C. The resulting mixture was stirred for 1 h at room temperature. The resulting mixture was extracted with CH₂Cl₂ (3×10 mL). The combined organic layers were washed with brine (30 mL), dried over anhydrous Na₂SO₄. After filtration, the filtrate was concentrated under reduced pressure. The residue was purified by silica gel column chromatography, eluted with PE/EA (27:73) to afford the title compound as yellow solid (910 mg, 76.8%). LCMS m/z=375 [M−H]⁻

### Preparation 89—5-chloro-2-(cyanoamino)-6-methyl-4-phenylpyridine-3-carbonitrile

To a stirred mixture of 5-chloro-3-cyano-6-methyl-4-phenylpyridin-2-yl trifluoromethanesulfonate (500 mg, 1.33 mmol) and cyanamide (223 mg, 5.31 mmol) in 1,4-dioxane (6.50 mL) was added XantPhos (120 mg, 0.133 mmol), Pd₂(dba)₃ (71.2 mg, 0.133 mmol) and DIEA (865 mg, 2.65 mmol) at room temperature under nitrogen atmosphere. The resulting mixture was stirred at 120° C. under nitrogen atmosphere for 1 h. The mixture was allowed to cool down to room temperature and concentrated under reduced pressure. The residue was purified by silica gel column chromatography, eluted with CH₂Cl₂/MeOH (6:1) to afford the title compound as yellow oil (200 mg, 56.1%). LCMS m/z=269 [M+H]⁺

### Preparation 90—6-chloro-5-methyl-N²-(1-methylpiperidin-4-yl)-7-phenylpyrido[2,3-d]pyrimidine-2,4-diamine

To a solution of 5-chloro-2-(cyanoamino)-6-methyl-4-phenylpyridine-3-carbonitrile (150 mg, 0.558 mmol) in 1,4-dioxane (1.50 mL) was added 1-methylpiperidin-4-amine (76.5 mg, 0.670 mmol) at room temperature. The resulting mixture was stirred for 1 h at 100° C. The mixture was allowed to cool down to room temperature. The residue was purified by trituration with DMF (4 mL) to afford the title compound as white solid (19.4 mg, 9.08%). ¹H NMR (400 MHz, DMSO-d₆) δ 7.60-7.59 (m, 2H), 7.52-7.43 (m, 3H), 7.01 (br, 2H), 6.67 (br, 1H), 3.79 (br, 1H), 2.80 (s, 3H), 2.74 (d, J=8.3 Hz, 2H), 2.15 (s, 3H), 1.93 (t, J=11.3 Hz, 2H), 1.79 (d, J=12.6 Hz, 2H), 1.52-1.50 (m, 2H). LCMS^{P} m/z=383 [M+H]⁺

### Example 49: N²-(1-methylpiperidin-4-yl)-7-(m-tolyl)pyrido[2,3-d]pyrimidine-2,4-diamine

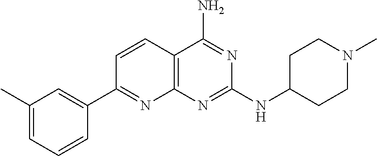

To a stirred mixture of Example 23 (40 mg, 0.137 mmol) in 1,4-Dioxane/H₂O (5/1, 2 mL) at room temperature was added 3-methylphenylboronic acid (20.4 mg, 0.151 mmol), CataCXium-A-Pd-G3 (5 mg, 0.007 mmol), CataCXium (6 mg, 0.017 mmol) and K₃PO₄ (87 mg, 0.411 mmol). The resulting mixture was stirred for 6 h at 100° C. under nitrogen atmosphere. The solution was cooled to room temperature and treated with mercaptoalkyl functionalized silica scavenger and then filtered. The filtrate was purified by Agela MP-Flash200^{Library} to afford the title compound as a solid (13.7 mg, 27.46%). ¹H NMR (300 MHz, DMSO-d6) δ 8.42 (d, J=8.3 Hz, 1H), 8.01 (s, 1H), 7.95 (d, J=7.8 Hz, 1H), 7.61 (d, J=8.2 Hz, 1H), 7.50-7.23 (m, 4H), 6.58 (s, 1H), 3.99-3.70 (m, 1H), 2.85-2.69 (m, 2H), 2.43 (s, 3H), 2.18 (s, 3H), 2.09-1.92 (m, 2H), 1.90-1.76 (m, 2H), 1.64-1.44 (m, 2H). LCMS^{Library G}: m/z=349 [M+H]⁺.

### Example 50-Example 114

The following Examples were synthesised in a similar manner to that described for Example 49, using the appropriate boronic acid or ester reagent:

| | | | LCMS |
|---|---|---|---|
| Example | Structure | ¹H NMR | m/z |
| Example 50 | 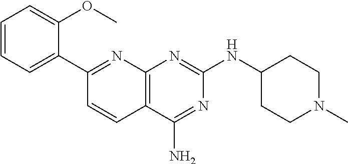 [SMILES: COc1ccccc1-c1ccc2c(N)nc(NC3CCN(C)CC3)nc2n1] | ¹H NMR (300 MHz, DMSO-d6) δ 8.32 (d, J = 8.3 Hz, 1H), 7.73 (dd, J = 7.6, 1.8 Hz, 1H), 7.50- 7.38 (m, 4H), 7.16 (d, J = 8.3 Hz, 1H), 7.08 (t, J = 7.4 Hz, 1H), 6.52 (s, 1H), 3.99-3.87 (m, 1H) 3.84 (s, 3H), 2.80- 2.72 (m, 2H), 2.17 (s, 3H), 2.02-1.88 (m, 2H), 1.88- 1.76 (m, 2H), 1.63-1.46 (m, 2H). | Library G: 365 [M + H]+ |
| Example 51 | 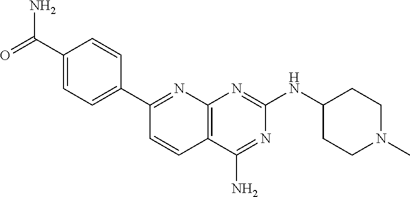 [SMILES: CN1CCC(Nc2nc(N)c3ccc(-c4ccc(C(N)=O)cc4)nc3n2)CC1] | | Library C: 378 [M + H]+ |
| Example 52 | 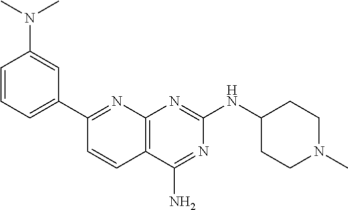 [SMILES: CN1CCC(Nc2nc(N)c3ccc(-c4cccc(N(C)C)c4)nc3n2)CC1] | | Library C: 378 [M + H]+ |
| Example 53 | 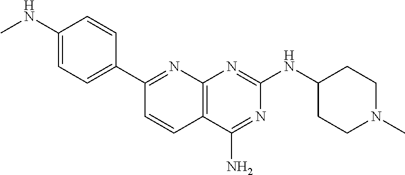 | | Library C: 364 [M + H]+ |
| Example 54 | 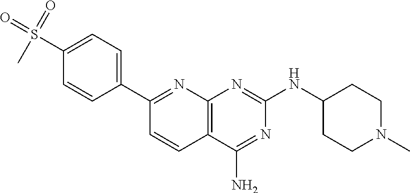 [SMILES: CN1CCC(Nc2nc(N)c3ccc(-c4ccc(S(C)(=O)=O)cc4)nc3n2)CC1] | | Library C: 413 [M + H]+ |
| Example 55 | 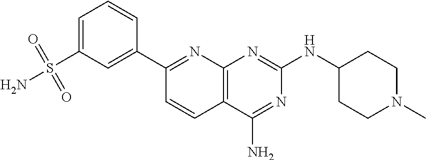 [SMILES: CN1CCC(Nc2nc(N)c3ccc(-c4cccc(S(N)(=O)=O)c4)nc3n2)CC1] | | Library C: 414 [M + H]+ |
| Example 56 | 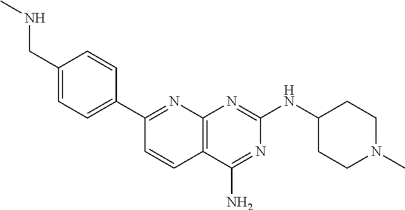 | | Library C: 378 [M + H]+ |
| Example 57 | 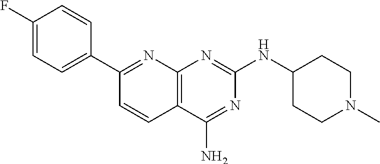 [SMILES: CN1CCC(Nc2nc(N)c3ccc(-c4ccc(F)cc4)nc3n2)CC1] | | Library C: 353 [M + H]+ |
| Example 58 | 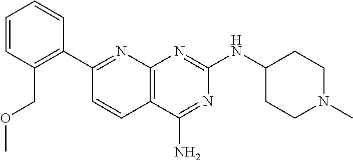 [SMILES: COCc1ccccc1-c1ccc2c(N)nc(NC3CCN(C)CC3)nc2n1] | | Library C: 379 [M + H]+ |
| Example 59 | 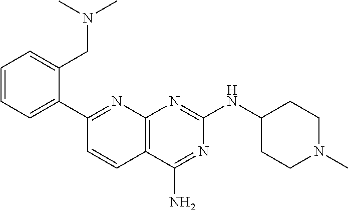 [SMILES: CN(C)Cc1ccccc1-c1ccc2c(N)nc(NC3CCN(C)CC3)nc2n1] | | Library G: 392 [M + H]+ |
| Example 60 | 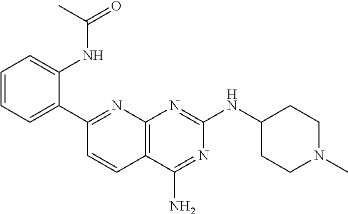 [SMILES: CC(=O)Nc1ccccc1-c1ccc2c(N)nc(NC3CCN(C)CC3)nc2n1] | | Library C: 392 [M + H]+ |
| Example 61 | 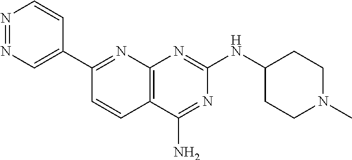 [SMILES: CN1CCC(Nc2nc(N)c3ccc(-c4ccnnc4)nc3n2)CC1] | | Library C: 337 [M + H]+ |
| Example 62 | 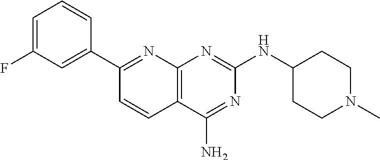 [SMILES: CN1CCC(Nc2nc(N)c3ccc(-c4cccc(F)c4)nc3n2)CC1] | | Library C: 353 [M + H]+ |
| Example 63 | 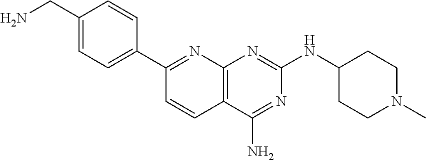 [SMILES: CN1CCC(Nc2nc(N)c3ccc(-c4ccc(CN)cc4)nc3n2)CC1] | | Library G: 364 [M + H]+ |
| Example 64 | 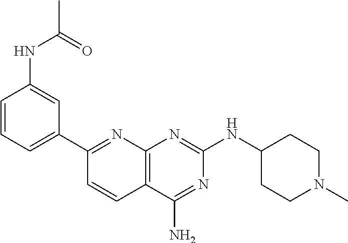 [SMILES: CC(=O)Nc1cccc(-c2ccc3c(N)nc(NC4CCN(C)CC4)nc3n2)c1] | | Library C: 392 [M + H]+ |
| Example 65 | 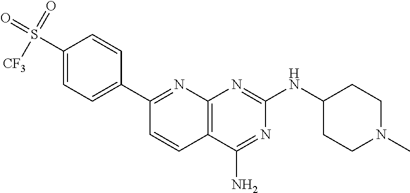 | | Library C: 414 [M + H]+ |
| Example 66 | 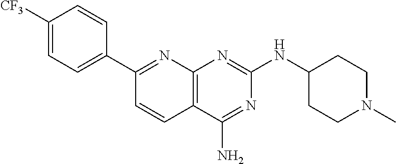 [SMILES: CN1CCC(Nc2nc(N)c3ccc(-c4ccc(C(F)(F)F)cc4)nc3n2)CC1] | | Library G: 403 [M + H]+ |
| Example 67 | 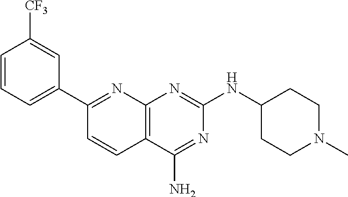 [SMILES: CN1CCC(Nc2nc(N)c3ccc(-c4cccc(C(F)(F)F)c4)nc3n2)CC1] | ¹H NMR (300 MHz, DMSO-d6) 8.55-8.45 (m, 3H), δ 7.86 (d, J = 8.0 Hz, 1H), 7.77 (t, J = 7.3 Hz, 2H), 7.52-7.42 (s, 2H), 6.58 (s, 1H), 3.99-3.81(m, 1H), 2.85-2.75 (m, 2H), 2.18 (s, 3H), 2.08-1.89 (m, 2H), 1.92-1.74 (m, 2H), 1.66- 1.43 (m, 2H). | Library C: 403 [M + H]+ |
| Example 68 | 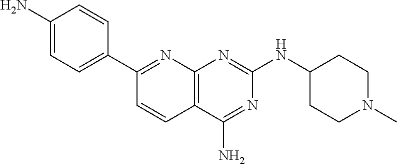 [SMILES: CN1CCC(Nc2nc(N)c3ccc(-c4ccc(N)cc4)nc3n2)CC1] | | Library C: 350 [M + H]+ |
| Example 69 | 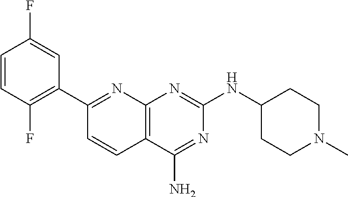 [SMILES: CN1CCC(Nc2nc(N)c3ccc(-c4cc(F)ccc4F)nc3n2)CC1] | | Library C: 371 [M + H]+ |
| Example 70 | 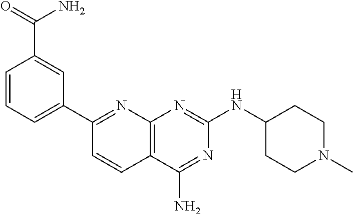 [SMILES: CN1CCC(Nc2nc(N)c3ccc(-c4cccc(C(N)=O)c4)nc3n2)CC1] | | Library C: 378 [M + H]+ |
| Example 71 | 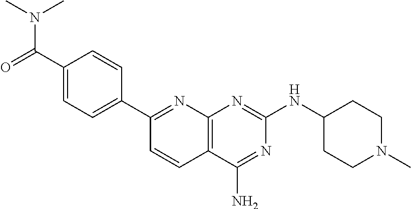 [SMILES: CN1CCC(Nc2nc(N)c3ccc(-c4ccc(C(=O)N(C)C)cc4)nc3n2)CC1] | | Library C: 406 [M + H]+ |
| Example 72 | 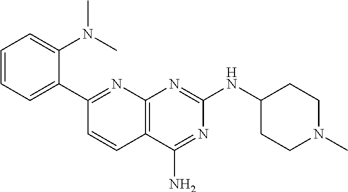 [SMILES: CN1CCC(Nc2nc(N)c3ccc(-c4ccccc4N(C)C)nc3n2)CC1] | | Library C: 378 [M + H]+ |
| Example 73 | 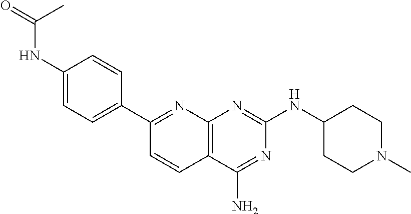 [SMILES: CC(=O)Nc1ccc(-c2ccc3c(N)nc(NC4CCN(C)CC4)nc3n2)cc1] | | Library C: 392 [M + H]+ |
| Example 74 | 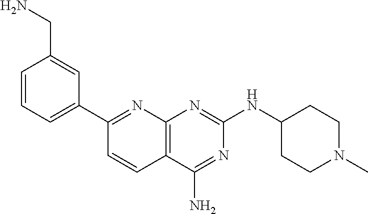 [SMILES: CN1CCC(Nc2nc(N)c3ccc(-c4cccc(CN)c4)nc3n2)CC1] | ¹H NMR (300 MHz, DMSO-d6) δ 8.43 (d, J = 8.3 Hz, 1H), 8.14 (s, 1H), 8.00 (d, J = 6.7 Hz, 1H), 7.62 (d, J = 8.4 Hz, 1H), 7.52-7.27 (m, 4H), 6.58 (s, 1H), 3.99-3.85(m, 1H), 3.83 (s, 2H), 2.83-2.69 (m, 2H), 2.18 (s, 3H), 2.09-1.89 (m, 2H), 1.90-1.74 (m, 2H), 1.65- 1.44 (m, 2H) | Library H: 364 [M + H]+ |
| Example 75 | 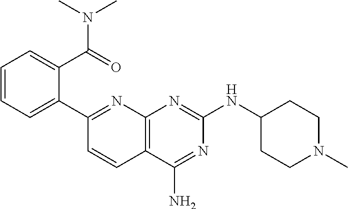 [SMILES: CN1CCC(Nc2nc(N)c3ccc(-c4ccccc4C(=O)N(C)C)nc3n2)CC1] | | Library C: 406 [M + H]+ |
| Example 76 | 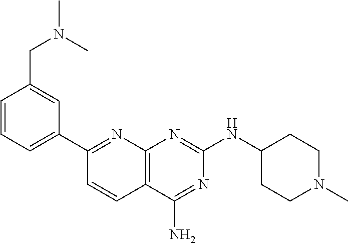 [SMILES: CN(C)Cc1cccc(-c2ccc3c(N)nc(NC4CCN(C)CC4)nc3n2)c1] | | Library C: 392 [M + H]+ |
| Example 77 | 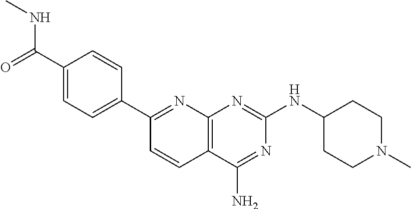 [SMILES: CNC(=O)c1ccc(-c2ccc3c(N)nc(NC4CCN(C)CC4)nc3n2)cc1] | | Library C: 392 [M + H]+ |
| Example 78 | 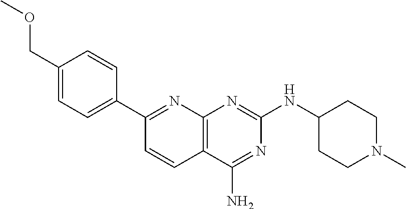 | | Library C: 379 [M + H]+ |
| Example 79 | 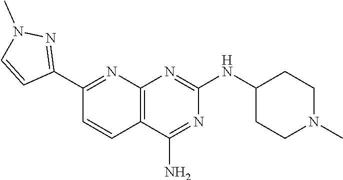 [SMILES: CN1CCC(Nc2nc(N)c3ccc(-c4ccn(C)n4)nc3n2)CC1] | | Library C: 339 [M + H]+ |
| Example 80 | 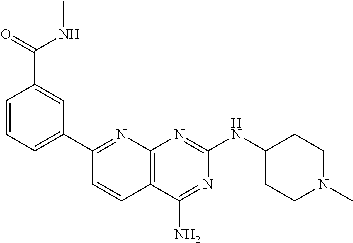 [SMILES: CNC(=O)c1cccc(-c2ccc3c(N)nc(NC4CCN(C)CC4)nc3n2)c1] | | Library C: 392 [M + H]+ |
| Example 81 | 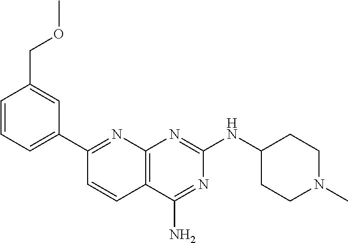 [SMILES: COCc1cccc(-c2ccc3c(N)nc(NC4CCN(C)CC4)nc3n2)c1] | | Library C: 379 [M + H]+ |
| Example 82 | 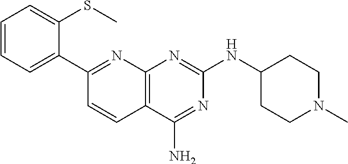 [SMILES: CSc1ccccc1-c1ccc2c(N)nc(NC3CCN(C)CC3)nc2n1] | ¹H NMR (300 MHz, DMSO-d6) δ 8.39 (d, J = 8.1 Hz, 1H), 7.52-7.42 (m, 5H), 7.28 (d, J = 6.0 Hz, 1H), 7.18 (d, J = 8.1 Hz, 1H), 6.58 (s, 1H), 3.96- 3.71(m, 1H),2.80-2.70 (m, 2H), 2.39 (s, 3H), 2.16 (s, 3H), 2.01-1.88 (m, 2H), 1.88- 1.75 (m, 2H), 1.61-1.45 (m, 2H). | Library G: 381 [M + H]+ |
| Example 83 | 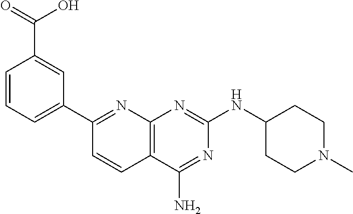 [SMILES: CN1CCC(Nc2nc(N)c3ccc(-c4cccc(C(=O)O)c4)nc3n2)CC1] | | Library C: 379 [M + H]+ |
| Example 84 | 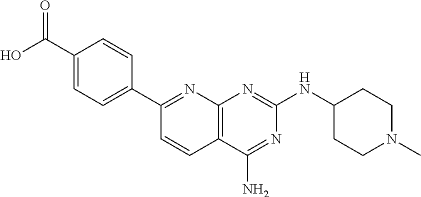 [SMILES: CN1CCC(Nc2nc(N)c3ccc(-c4ccc(C(=O)O)cc4)nc3n2)CC1] | | Library C: 379 [M + H]+ |
| Example 85 | 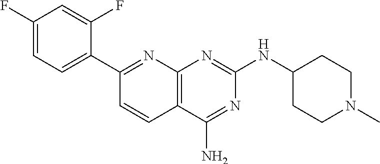 [SMILES: CN1CCC(Nc2nc(N)c3ccc(-c4ccc(F)cc4F)nc3n2)CC1] | | Library G: 371 [M + H]+ |
| Example 86 | 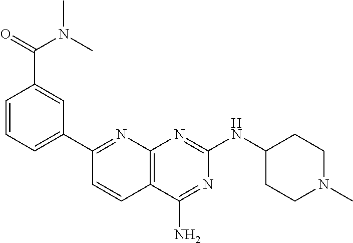 [SMILES: CN1CCC(Nc2nc(N)c3ccc(-c4cccc(C(=O)N(C)C)c4)nc3n2)CC1] | | Library C: 406 [M + H]+ |
| Example 87 | 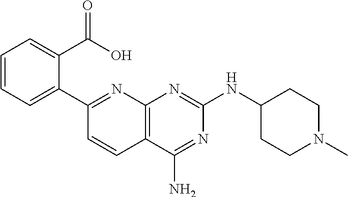 [SMILES: CN1CCC(Nc2nc(N)c3ccc(-c4ccccc4C(=O)O)nc3n2)CC1] | | Library C: 379 [M + H]+ |
| Example 88 | 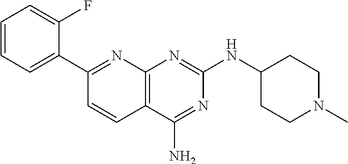 [SMILES: CN1CCC(Nc2nc(N)c3ccc(-c4ccccc4F)nc3n2)CC1] | | Library C: 353 [M + H]+ |
| Example 89 | 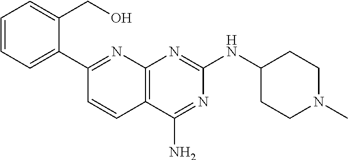 [SMILES: CN1CCC(Nc2nc(N)c3ccc(-c4ccccc4CO)nc3n2)CC1] | | Library C: 365 [M + H]+ |
| Example 90 | 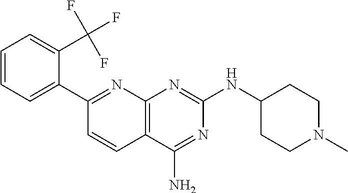 [SMILES: CN1CCC(Nc2nc(N)c3ccc(-c4ccccc4C(F)(F)F)nc3n2)CC1] | | Library C: 403 [M + H]+ |
| Example 91 | 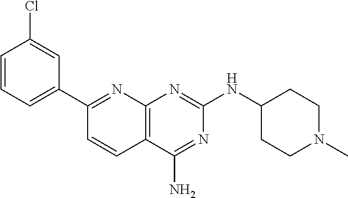 [SMILES: CN1CCC(Nc2nc(N)c3ccc(-c4cccc(Cl)c4)nc3n2)CC1] | ¹H NMR (300 MHz, DMSO-d6) δ 8.46 (d, J = 8.2 Hz, 1H), 8.22 (s, 1H), 8.15 (d, J = 5.0 Hz, 1H), 7.68 (d, J = 8.2 Hz, 1H), 7.62-7.29 (m, 4H),6.58 (s, 1H), 3.98-3.71 (m, 1H),2.82- 2.67 (m, 2H), 2.18 (s, 3H), 2.08-1.90 (m, 2H), 1.90- 1.78 (m, 2H), 1.66-1.42 (m, 2H). | Library C: 369 [M + H]+ |
| Example 92 | 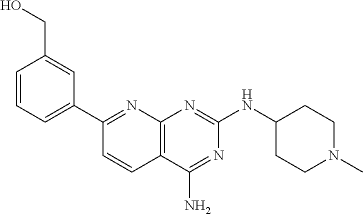 [SMILES: CN1CCC(Nc2nc(N)c3ccc(-c4cccc(CO)c4)nc3n2)CC1] | | Library C: 365 [M + H]+ |
| Example 93 | 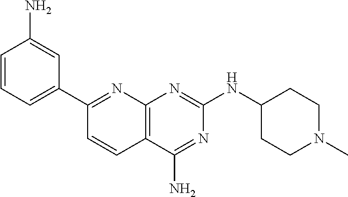 [SMILES: CN1CCC(Nc2nc(N)c3ccc(-c4cccc(N)c4)nc3n2)CC1] | | Library C: 350 [M + H]+ |
| Example 94 | 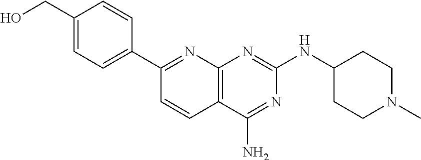 [SMILES: CN1CCC(Nc2nc(N)c3ccc(-c4ccc(CO)cc4)nc3n2)CC1] | | Library C: 365 [M + H]+ |
| Example 95 | 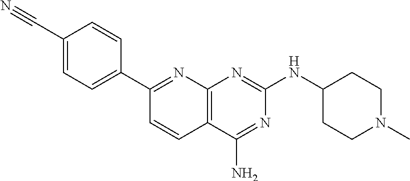 [SMILES: CN1CCC(Nc2nc(N)c3ccc(-c4ccc(C#N)cc4)nc3n2)CC1] | | Library C: 360 [M + H]+ |
| Example 96 | 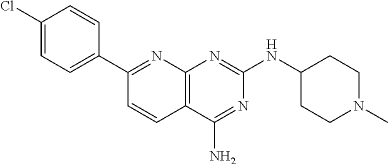 [SMILES: CN1CCC(Nc2nc(N)c3ccc(-c4ccc(Cl)cc4)nc3n2)CC1] | | Library G: 369 [M + H]+ |
| Example 97 | 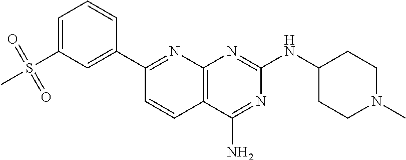 | | Library C: 413 [M + H]+ |
| Example 98 | 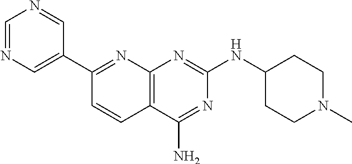 [SMILES: CN1CCC(Nc2nc(N)c3ccc(-c4cncnc4)nc3n2)CC1] | | Library C: 337 [M + H]+ |
| Example 99 | 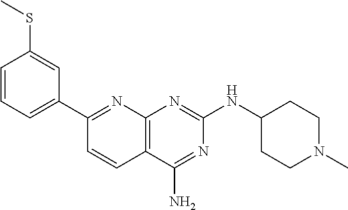 [SMILES: CSc1cccc(-c2ccc3c(N)nc(NC4CCN(C)CC4)nc3n2)c1] | ¹H NMR (300 MHz, DMSO-d6) δ 8.44 (d, J = 8.3 Hz, 1H), 8.03 (s, 1H), 7.93 (d, J = 7.6 Hz, 1H), 7.64 (d, J = 8.3 Hz, 1H), 7.59-7.24 (m, 4H), 6.60 (s, 1H), 3.98-3.77 (m, 1H), 2.82- 2.70 (m, 2H), 2.57 (s, 3H), 2.18 (s, 3H), 2.07-1.91 (m, 2H), 1.90-1.73 (m, 2H), 1.66- 1.43 (m, 2H). | Library G: 381 [M + H]+ |
| Example 100 | 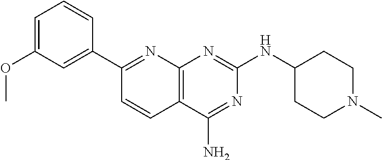 [SMILES: COc1cccc(-c2ccc3c(N)nc(NC4CCN(C)CC4)nc3n2)c1] | | Library C: 365 [M + H]+ |
| Example 101 | 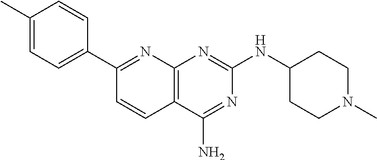 | | Library G: 349 [M + H]+ |
| Example 102 | 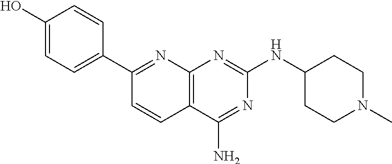 [SMILES: CN1CCC(Nc2nc(N)c3ccc(-c4ccc(O)cc4)nc3n2)CC1] | | Library G: 351 [M + H]+ |
| Example 103 | 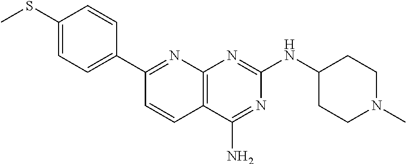 | ¹H NMR (300 MHz, DMSO-d6) δ 8.41 (d, J = 8.3 Hz, 1H), 8.14 (d, J = 8.5 Hz, 2H), 7.61 (d, J = 8.1 Hz, 1H), 7.38 (d, J = 8.4 Hz, 4H), 6.58 (s, 1H), 3.90- 3.73 (m, 1H), 2.83-2.69 (m, 2H), 2.55 (s, 3H), 2.18 (s, 3H), 2.06-1.90 (m, 2H), 1.88- 1.76 (m, 2H), 1.63-1.46 (m, 2H). | Library G: 381 [M + H]+ |
| Example 104 | 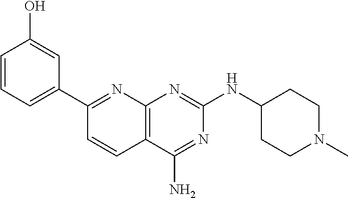 [SMILES: CN1CCC(Nc2nc(N)c3ccc(-c4cccc(O)c4)nc3n2)CC1] | | Library C: 351 [M + H]+ |
| Example 105 | 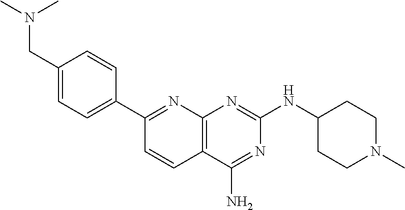 | | Library C: 392 [M + H]+ |
| Example 106 | 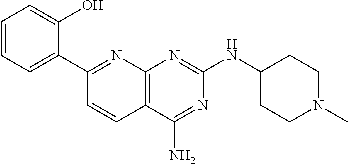 [SMILES: CN1CCC(Nc2nc(N)c3ccc(-c4ccccc4O)nc3n2)CC1] | | Library C: 351 [M + H]+ |
| Example 107 | 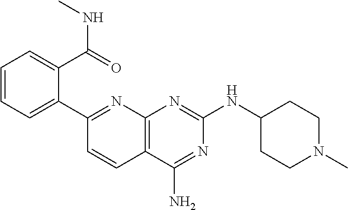 [SMILES: CNC(=O)c1ccccc1-c1ccc2c(N)nc(NC3CCN(C)CC3)nc2n1] | | Library C: 392 [M + H]+ |
| Example 108 | 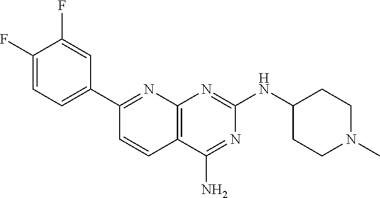 [SMILES: CN1CCC(Nc2nc(N)c3ccc(-c4ccc(F)c(F)c4)nc3n2)CC1] | | Library C: 371 [M + H]+ |
| Example 109 | 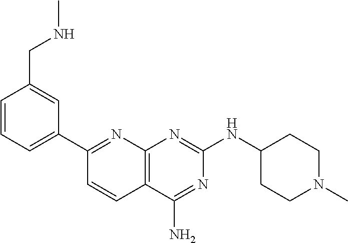 [SMILES: CNCc1cccc(-c2ccc3c(N)nc(NC4CCN(C)CC4)nc3n2)c1] | | Library C: 378 [M + H]+ |
| Example 110 | 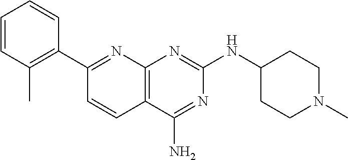 [SMILES: Cc1ccccc1-c1ccc2c(N)nc(NC3CCN(C)CC3)nc2n1] | ¹H NMR (300 MHz, DMSO-d6) δ 8.40 (d, J = 8.2 Hz, 1H), 7.39 (s, 2H), 7.36-7.24 (m, 4H), 7.11 (d, J = 8.2 Hz, 1H), 6.58 (s, 1H), 3.90-3.73 (m, 1H), 2.81-2.68 (m, 2H), 2.34 (s, 3H), 2.16 (s, 3H), 2.02-1.88 (m, 2H), 1.87-1.76 (m, 2H), 1.63-1.43 (m, 2H). | Library C: 349 [M + H]+ |
| Example 111 | 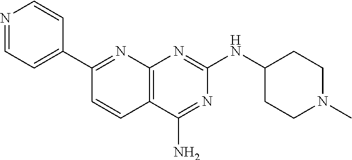 [SMILES: CN1CCC(Nc2nc(N)c3ccc(-c4ccncc4)nc3n2)CC1] | | Library C: 336 [M + H]+ |
| Example 112 | 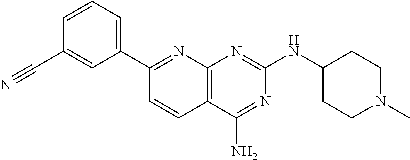 [SMILES: CN1CCC(Nc2nc(N)c3ccc(-c4cccc(C#N)c4)nc3n2)CC1] | ¹H NMR (300 MHz, DMSO-d6) δ 8.59 (s, 1H), 8.51 (t, J = 8.9 Hz, 2H), 7.96 (d, J = 7.7 Hz, 1H), 7.80-7.69 (m, 2H), 7.46 (s, 2H),6.58 (s, 1H), 3.96- 3.79 (m, 1H), 2.78 (s, 2H),2.18 (s, 3H), 2.06-1.91 (m, 2H), 1.90-1.76 (m, 2H), 1.64-1.45 (m, 2H). | Library C: 360 [M + H]+ |
| Example 113 | 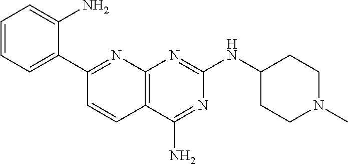 [SMILES: CN1CCC(Nc2nc(N)c3ccc(-c4ccccc4N)nc3n2)CC1] | | Library C: 350 [M + H]+ |
| Example 114 | 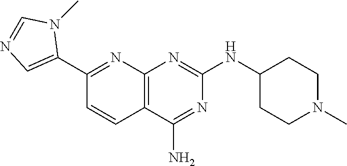 [SMILES: CN1CCC(Nc2nc(N)c3ccc(-c4cncn4C)nc3n2)CC1] | | Library C: 339 [M + H]+ |

### Example 115: 7-(2-chlorophenyl)-N²-(1-methylpiperidin-4-yl) pyrido[2,3-d]pyrimidine-2,4-diamine

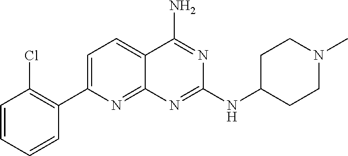 [SMILES: CN1CCC(Nc2nc(N)c3ccc(-c4ccccc4Cl)nc3n2)CC1]

To a stirred mixture of Example 23 (40 mg, 0.137 mmol) in 1,4-Dioxane/H₂O (5/1, 2 ml-) at room temperature was added 2-chlorophenylboronic acid (23.5 mg, 0.151 mmol), Xphos G₂ (5 mg, 0.006 mmol) and K₃PO₄ (87.00 mg, 0.411 mmol). The resulting mixture was stirred for 6 h at 110° C. under nitrogen atmosphere. The solution was cooled to room temperature and treated with mercaptoalkyl functionalized silica scavenger and then filtered. The filtrate was purified by Agela MP-Flash200^{Library} to afford the title compound as a solid (3.3 mg, 6.55%). LCMS^{Library C}: m/z=369 [M+H]⁺.

### Example 116-Example 118

The following Examples were synthesised in a similar manner to that described for Example 115, using the appropriate boronic acid or ester reagent:

| | | | LCMS |
|---|---|---|---|
| Example | Structure | ¹H NMR | m/z |
| Example 116 | 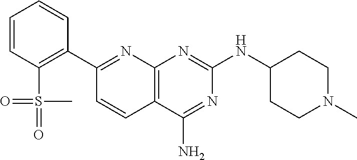 [SMILES: CN1CCC(Nc2nc(N)c3ccc(-c4ccccc4S(C)(=O)=O)nc3n2)CC1] | | Library C: 413 [M + H]+ |
| Example 117 | 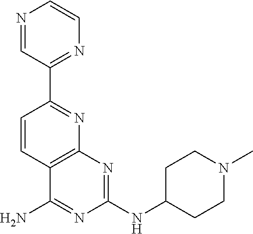 [SMILES: CN1CCC(Nc2nc(N)c3ccc(-c4cnccn4)nc3n2)CC1] | | Library C: 337 [M + H]+ |
| Example 118 | 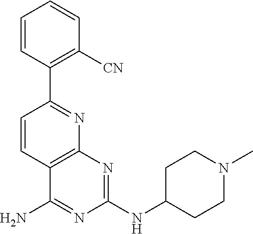 [SMILES: CN1CCC(Nc2nc(N)c3ccc(-c4ccccc4C#N)nc3n2)CC1] | | Library C: 360 [M + H]+. |

### Example 119: N²-(1-methylpiperidin-4-yl)-7-(oxazol-2-yl)pyrido[2,3-d]pyrimidine-2,4-diamine

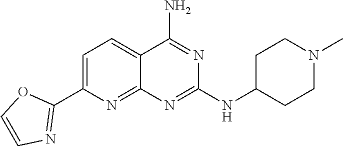 [SMILES: CN1CCC(Nc2nc(N)c3ccc(-c4ncco4)nc3n2)CC1]

To a stirred mixture of Example 23 (40 mg, 0.137 mmol) in Toluene (2 ml-) at room temperature was added 2-(tributylstannyl)-1,3-oxazole (53.82 mg, 0.151 mmol) and Pd(PPh₃)₄ (5 mg, 0.004 mmol). The resulting mixture was stirred for 6 h at 110° C. under nitrogen atmosphere. The solution was cooled to room temperature and treated with mercaptoalkyl functionalized silica scavenger and then filtered. The filtrate was purified by Agela MP-Flash200^{Library} to afford the title compound as a solid (3.9 mg, 8.77%). LCMS^{Library} C: m/z=326 [M+H]⁺.

### Example 120: 5-methyl-N2-(3-morpholinopropyl)-7-phenylpyrido[2,3-d]pyrimidine-2,4-diamine

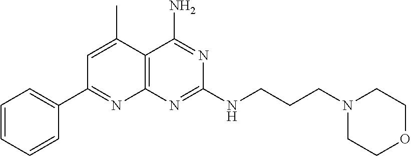 [SMILES: Cc1cc(-c2ccccc2)nc2nc(NCCCN3CCOCC3)nc(N)c12]

### Preparation 91—2-chloro-4-methyl-6-phenylnicotinonitrile

To a solution of 2,6-dichloro-4-methylnicotinonitrile (20.0 g, 107 mmol) and phenyl boronic acid (10.4 g, 85.6 mmol) in 1,4-dioxane (150 mL) and H₂O (150 mL) at room temperature were added Pd(dppf)Cl₂ (7.82 g, 10.7 mmol) and K₃PO₄ (45.4 g, 214 mmol). The resulting mixture was stirred under nitrogen atmosphere at room temperature for 1 h. The resulting mixture was concentrated under reduced pressure. The resulting mixture was filtered, the filter cake was washed with water (3×50 mL). The solid was purified by trituration with MeOH (3×100 mL) to afford title compound (22.3 g, crude) as a reddish solid. LCMS m/z=229 [M+H]⁺

### Preparation 92—N-(3-cyano-4-methyl-6-phenylpyridin-2-yl)cyanamide

To a solution of 2-chloro-4-methyl-6-phenylnicotinonitrile (2.00 g, 8.75 mmol), Cs₂CO₃ (5.70 g, 17.5 mmol) and cyanamide (0.740 g, 17.5 mmol) in 1,4-dioxane (30.0 mL) at room temperature were added Brettphos Pd G3 (0.790 g, 0.875 mmol) and Brettphos (0.470 g, 0.875 mmol). The mixture was stirred under nitrogen atmosphere at 100° C. for 2 h. The mixture was allowed to cool down to room temperature. The resulting mixture was filtered, the filter cake was washed with DCM (3×100 mL). The combined filtrate was concentrated under reduced pressure. The residue was purified by HPLC^{B} to afford the title compound (600 mg, 29.30%) as a yellow green solid. LCMS m/z=235 [M+H]⁺.

### Preparation 93—5-methyl-N²-(3-morpholinopropyl)-7-phenylpyrido[2,3-d]pyrimidine-2,4-diamine

To a solution of N-(3-cyano-4-methyl-6-phenylpyridin-2-yl)cyanamide (100 mg, 0.427 mmol) in EtOH (2.0 mL) at room temperature was added 3-morpholinopropan-1-amine (185 mg, 1.28 mmol). The resulting mixture was stirred at 100° C. for 2 h. The mixture was allowed to cool down to room temperature and concentrated under reduced pressure. The residue was purified by Prep-HPLC^{AF} to afford the title compound (72.8 mg, 45.10%) as a white solid. ¹H NMR (400 MHz, DMSO-d₆) δ 8.19-8.11 (m, 2H), 7.55-7.40 (m, 4H), 7.04-6.51 (m, 3H), 3.59 (t, J=4.6 Hz, 4H), 3.41-3.36 (m, 2H), 2.77 (s, 3H), 2.41-2.30 (m, 6H), 1.77-1.65 (m, 2H). LCMS^{C} m/z=379 [M+H]1

### Example 121: 5,6,7-trimethyl-N2-(1-methylpiperidin-4-yl)pyrido[2,3-d]pyrimidine-2,4-diamine

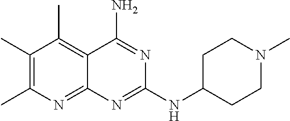

### Preparation 94—N-(3-cyano-4,5,6-trimethylpyridin-2-yl)cyanamide

A solution of 2-chloro-4,5,6-trimethylnicotinonitrile (200 mg, 1.10 mmol) and cyanamide, monosodium salt (141 mg, 2.21 mmol) in DMSO (0.5 mL) was stirred at 100° C. for 2 h. The mixture was allowed to cool down to room temperature. The residue was purified by Prep-HPLC^{B} to afford the title compound as a light-yellow solid (110 mg, 53.3%). LCMS m/z=187 [M+H]⁺

### Preparation 95—5,6,7-trimethyl-N2-(1-methylpiperidin-4-yl)pyrido[2,3-d]pyrimidine-2,4-diamine

A solution of N-(3-cyano-4,5,6-trimethylpyridin-2-yl)cyanamide (90.0 mg, 0.483 mmol) and 1-methylpiperidin-4-amine (110 mg, 0.966 mmol) in EtOH (5.00 mL) was stirred at 100° C. for 2.5 h. The mixture was allowed to cool down to room temperature. The resulting mixture was concentrated under reduced pressure. The residue was purified by Prep-HPLC^{AU} to afford the title compound as a white solid (78.7 mg, 54.2%). ¹H NMR (400 MHz, DMSO-d₆) δ 6.68 (br, 2H), 6.23 (br, 1H), 3.81-3.68 (m, 1H), 2.74-2.72 (m, 2H), 2.57 (s, 3H), 2.44 (s, 3H), 2.17 (s, 3H), 2.16 (s, 3H), 1.96-1.91 (m, 2H), 1.81-1.78 (m, 2H), 1.57-1.42 (m, 2H). LCMS^{C} m/z=301 [M+H]⁺

### Example 122: methyl 4-amino-2-[(1-methylpiperidin-4-yl)amino]pyrido[2,3-d]pyrimidine-6-carboxylate

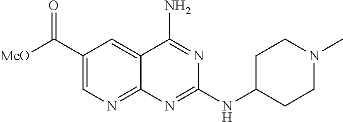 [SMILES: COC(=O)c1cnc2nc(NC3CCN(C)CC3)nc(N)c2c1]

To a stirred solution of Example 20 (1.50 g, 4.45 mmol) in MeOH (200 mL) and DMF (4.0 mL) were added Pd(dppf)Cl₂·CH₂Cl₂ (362 mg, 0.445 mmol) and Et₃N (1.24 mL, 8.90 mmol). The resulting mixture was stirred under carbon monoxide atmosphere (10 atm) at 100° C. overnight. The mixture was allowed to cool down to room temperature. The resulting mixture was filtered, the filter cake was washed with MeOH (3×10 mL). The filtrate was concentrated under reduced pressure. The residue was purified by Prep-HPLC^{B} to afford the title compound (900 mg, 63.8%) as a brown solid. ¹H NMR (400 MHz, DMSO-d₆) δ 9.04 (s, 1H), 8.96 (s, 1H), 7.75 (br, 2H), 7.35-7.00 (m, 1H), 3.87 (s, 3H), 3.86-3.83 (m, 1H), 2.75-2.73 (m, 2H), 2.16 (s, 3H), 1.96-1.91 (m, 2H), 1.85-1.72 (m, 2H), 1.61-1.39 (m, 2H). LCMS^{C} m/z=317 [M+H]⁺

### Example 123: {4-amino-2-[(1-methylpiperidin-4-yl)amino]pyrido[2,3-d]pyrimidin-6-yl}methanol

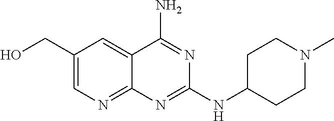 [SMILES: CN1CCC(Nc2nc(N)c3cc(CO)cnc3n2)CC1]

To a stirred mixture of Example 122 (200 mg, 0.632 mmol) in THF (4.0 mL) was added LiAlH₄ (474 μL, 0.948 mmol, 2.0 M in THF) dropwise at 0° C. The resulting mixture was stirred at 0° C. for 1 h. The reaction was quenched with water at 0° C. The resulting mixture was concentrated under reduced pressure. The residue was purified by Prep-HPLC^{AU} to afford the title compound (22.8 mg, 12.5%) as an off-white solid. ¹H NMR (400 MHz, DMSO-d₆) δ 8.57 (s, 1H), 8.29 (s, 1H), 7.37 (br, 2H), 6.52 (br, 1H), 5.22 (t, J=5.4 Hz, 1H), 4.50 (d, J=5.2 Hz, 2H), 3.98-3.61 (m, 1H), 2.79-2.70 (m, 2H), 2.16 (s, 3H), 1.99-1.89 (m, 2H), 1.86-1.78 (m, 2H), 1.65-1.32 (m, 2H). LCMS^{C} m/z=289 [M+H]⁺

### Example 124: 5-methyl-7-phenyl-N²-(quinuclidin-4-yl)pyrido[2,3-d]pyrimidine-2,4-diamine

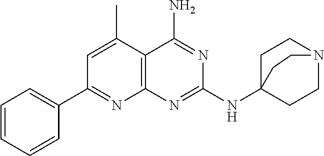 [SMILES: Cc1cc(-c2ccccc2)nc2nc(NC34CCN(CC3)CC4)nc(N)c12]

To a stirred solution of N-(3-cyano-4-methyl-6-phenylpyridin-2-yl)cyanamide (50.0 mg, 0.213 mmol) in EtOH (1.0 mL) were added quinuclidin-4-amine hydrochloride (160 mg, 1.28 mmol) and Et₃N (534 μL, 3.84 mmol). The resulting mixture was stirred at 100° C. for 4 days. The resulting mixture was concentrated under reduced pressure. The residue was purified by Prep-HPLC^{AS} to afford the title compound (19.4 mg, 25.30%) as a white solid. ¹H NMR (400 MHz, DMSO-d₆) δ 8.19-8.10 (m, 2H), 7.56-7.46 (m, 3H), 7.43 (s, 1H), 6.86 (s, 2H), 6.06 (s, 1H), 2.96-2.82 (m, 6H), 2.77 (s, 3H), 2.07-1.87 (m, 6H). LCMS^{O} m/z=361 [M+H]⁺

### Example 125: 1-{4-amino-2-[(1-methylpiperidin-4-yl)amino]pyrido[2,3-d]pyrimidin-6-yl}ethenone

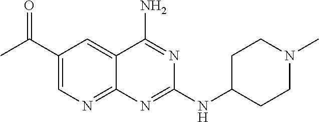 [SMILES: CC(=O)c1cnc2nc(NC3CCN(C)CC3)nc(N)c2c1]

To a stirred solution of Example 20 (600 mg, 1.78 mmol) and tributyl(1-ethoxyethenyl)stannane (1.29 g, 3.56 mmol) in DMF (9.0 mL) was added Pd(PPh₃)₄ (206 mg, 0.178 mmol). The resulting mixture was stirred under nitrogen atmosphere at 100° C. overnight. The mixture was allowed to cool down to room temperature. To the above mixture was added aq. HCl (1.0 M, 9.0 mL) at room temperature and stirred for additional 1 h at room temperature. The mixture was basified pH 9 with saturated NaHCO₃ (aq.). and extracted with EtOAc (50 mL×3). The combined organic phases were washed with brine, dried over Na₂SO₄, filtered and concentrated under reduced pressure. The residue was purified by Prep-HPLC^{B} to afford the title compound (400 mg, 74.7%) as a yellow solid. ¹H NMR (400 MHz, DMSO-d₆) δ 9.08 (s, 1H), 8.95 (s, 1H), 7.76 (br, 2H), 7.39-6.95 (m, 1H), 4.02-3.72 (m, 1H), 2.77-2.74 (m, 2H), 2.56 (s 3H), 2.16 (s, 3H), 1.97-1.94 (m, 2H), 1.81-1.79 (m, 2H), 1.57-1.49 (m, 2H). LCMS^{H} m/z=301 [M+H]⁺

### Example 126: 1-{4-amino-2-[(1-methylpiperidin-4-yl)amino]pyrido[2,3-d]pyrimidin-6-yl}ethanol

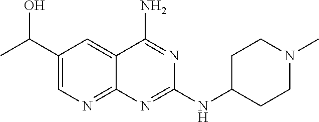 [SMILES: CC(O)c1cnc2nc(NC3CCN(C)CC3)nc(N)c2c1]

To a stirred solution of Example 125 (150 mg, 0.499 mmol) in MeOH (3.0 mL) was added NaBH₄ (37.8 mg, 0.999 mmol) at room temperature. The resulting mixture was stirred at room temperature overnight. The reaction was quenched with water at 0° C. The resulting mixture was extracted with EtOAc (50 mL×3). The combined organic phases were washed with brine, filtered and concentrated under reduce pressure. The residue was purified by Prep-HPLC^{AS} to afford the title compound (20.4 mg, 13.5%) as an off-white solid. ¹H NMR (400 MHz, DMSO-d₆) δ 8.60 (s, 1H), 8.30 (s, 1H), 7.38 (br, 2H), 6.48 (br, 1H), 5.25 (d, J=4.0 Hz, 1H), 4.78-4.75 (m, 1H), 3.80-3.79 (m, 1H), 2.75-2.72 (m, 2H), 2.16 (s, 3H), 1.99-1.89 (m, 2H), 1.86-1.77 (m, 2H), 1.60-1.42 (m, 2H), 1.40 (d, J=6.4 Hz, 3H). LCMS^{H} m/z=303 [M+H]⁺

### Example 127: 6-phenyl-N³-(quinuclidin-4-yl)-8,9-dihydro-7H-cyclopenta[4,5]pyrido[2,3-d]pyrimidine-1,3-diamine

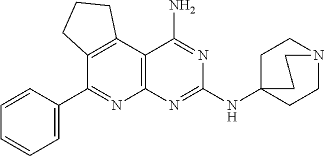 [SMILES: Nc1nc(NC23CCN(CC2)CC3)nc2nc(-c3ccccc3)c3c(c12)CCC3]

To a stirred solution of N-(4-cyano-1-phenyl-6,7-dihydro-5H-cyclopenta[c]pyridin-3-yl)cyanamide (30 mg, 0.115 mmol) and quinuclidin-4-amine hydrochloride (87.2 mg, 0.690 mmol) in EtOH (1.0 mL) at room temperature was added Et₃N (175 mg, 1.73 mmol). The resulting mixture was stirred at 100° C. for 30 h. The resulting mixture was concentrated under reduced pressure. The residue was purified by Prep-HPLC^{B} to afford the title compound (5.3 mg, 10.67%) as a white solid formate salt. ¹H NMR (400 MHz, DMSO-d₆) δ 8.29 (s, 1H), 7.82-7.72 (m, 2H), 7.54-7.41 (m, 3H), 6.78 (br, 2H), 6.23 (br, 1H), 3.40-3.35 (m, 2H), 3.10-2.96 (m, 8H), 2.18-2.10 (m, 8H). LCMS^{R} m/z=387 [M+H]⁺

### Example 128: 6-chloro-5-methyl-N2-(3-morpholinopropyl)-7-phenylpyrido[2,3-d]pyrimidine-2,4-diamine

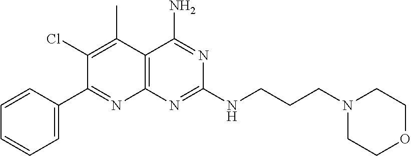 [SMILES: Cc1c(Cl)c(-c2ccccc2)nc2nc(NCCCN3CCOCC3)nc(N)c12]

A solution of Example 120 (25.0 mg, 0.066 mmol) and 1-chloropyrrolidine-2,5-dione (26.5 mg, 0.198 mmol) in THF (1.0 mL) and MeOH (1.0 mL) was stirred at room temperature for 2 h. The resulting mixture was concentrated under reduced pressure. The residue was dissolved in water (5 mL) and extracted with EtOAc (3×10 mL). The combined organic layers were dried over anhydrous Na₂SO₄. After filtration, the filtrate was concentrated under reduced pressure. The residue was purified by Prep-HPLC^{G} to afford the title compound (3.3 mg, 12.1%) as a yellow solid. ¹H NMR (400 MHz, Methanol-d₄) δ 7.68-7.60 (m, 2H), 7.51-7.41 (m, 3H), 3.70 (t, J=4.7 Hz, 4H), 3.48 (t, J=6.8 Hz, 2H), 2.89 (s, 3H), 2.53-2.43 (m, 6H), 1.89-1.77 (m, 2H). LCMS^{C} m/z=413 [M+H]⁺

### Example 129: N³-(3-morpholinopropyl)-6-phenyl-8,9-dihydro-7H-cyclopenta[4,5]pyrido[2,3-d]pyrimidine-1,3-diamine

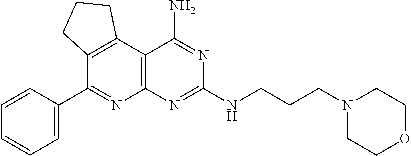 [SMILES: Nc1nc(NCCCN2CCOCC2)nc2nc(-c3ccccc3)c3c(c12)CCC3]

### Preparation 96—1,3-dihydroxy-6,7-dihydro-5H-cyclopenta[c]pyridine-4-carbonitrile

To a stirred solution of ethyl 2-oxocyclopentane-1-carboxylate (25.0 g, 160 mmol) in MeOH (125 mL) were added cyanoacetamide (14.1 g, 168 mmol) and KOH (9.43 g, 168 mmol, 1.05 equiv). The resulting mixture was stirred at 70° C. for 1 h. The mixture was allowed to cool down to room temperature and acidified to pH 1 with HCl (aq. 1 M). The precipitated solids were collected by filtration and washed with water (200 mL). The resulting solid was dried under vacuum to afford the title compound (9.5 g, 33.6%) as a white solid.

### Preparation 97—4-cyano-6,7-dihydro-5H-cyclopenta[c]pyridine-1,3-diyl bis(trifluoromethanesulfonate)

To a solution of 1,3-dihydroxy-6,7-dihydro-5H-cyclopenta[c]pyridine-4-carbonitrile (4.00 g, 22.7 mmol) and DIEA (8.80 g, 68.1 mmol) in DCM (40.0 mL) was added Tf₂O (38.4 g, 136 mmol) dropwise at 0° C. The resulting mixture was stirred at room temperature for 1 h. The reaction was quenched with water at 0° C. The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE/EA (3:1) to afford the title compound (6.4 g, 64.0%) as a brown oil. LCMS m/z=441 [M+H]⁺

### Preparation 98—4-cyano-1-phenyl-6,7-dihydro-5H-cyclopenta[c]pyridin-3-yl trifluoromethanesulfonate

To a stirred solution of 4-cyano-6,7-dihydro-5H-cyclopenta[c]pyridine-1,3-diyl bis(trifluoromethanesulfonate) (5.00 g, 11.3 mmol) and phenyl boronic acid (692 mg, 5.67 mmol) in 1,4-dioxane (30.0 mL) and H₂O (10.0 mL) were added Pd(dppf)Cl₂ (830 mg, 1.13 mmol) and K₃PO₄ (4.82 g, 22.7 mmol). The resulting mixture was stirred at room temperature for 1 h. The resulting mixture was filtered through a short pad of Celite. The pad was washed with CH₂Cl₂ (2×20 mL). The combined filtrate was concentrated under reduced pressure. The resulting mixture was diluted with water (200 mL) and extracted with CH₂Cl₂ (3×200 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (9:1) to afford the title compound (1.50 g, 35.9%) as a white solid. LCMS m/z=369 [M+H]⁺

### Preparation 99—N-(4-cyano-1-phenyl-6,7-dihydro-5H-cyclopenta[c]pyridin-3-yl)cyanamide

To a mixture of 4-cyano-1-phenyl-6,7-dihydro-5H-cyclopenta[c]pyridin-3-yl trifluoromethanesulfonate (1.35 g, 3.66 mmol) and cyanamide (360 mg, 8.56 mmol) in dioxane (23.0 mL) were added Brettphos Pd G3 (332 mg, 0.367 mmol), Brettphos (197 mg, 0.367 mmol) and Cs₂CO₃ (2.39 g, 7.33 mmol). The resulting mixture was stirred at 100° C. under nitrogen atmosphere for 1 h. The mixture was allowed to cool down to room temperature. The resulting mixture was filtered through a short pad of Celite. The pad was washed with MeOH (3×20 mL). The combined filtrate was concentrated under reduced pressure. The residue was purified by Prep-HPLC^{B} to afford the title compound (340 mg, 35.6%) as a brown solid. LCMS m/z=261 [M+H]⁺

### Preparation 100—N³-(3-morpholinopropyl)-6-phenyl-8,9-dihydro-7H-cyclopenta[4,5]pyrido[2,3-d]pyrimidine-1,3-diamine

To a stirred solution of N-(4-cyano-1-phenyl-6,7-dihydro-5H-cyclopenta[c]pyridin-3-yl)cyanamide (150 mg, 0.576 mmol) in EtOH (7.50 mL) was added 4-morpholinepropanamine (220 μL, 1.52 mmol). The resulting mixture was stirred at 100° C. for 1 h. The resulting mixture was concentrated under reduced pressure. The residue was purified by Prep-HPLC^{B} to afford the title compound (36.1 mg, 15.5%) as a yellow solid. ¹H NMR (400 MHz, DMSO-d₆) δ 7.85-7.68 (m, 2H), 7.65-7.37 (m, 3H), 6.75 (br, 2H), 6.57 (br, 1H), 3.58 (t, J=4.6 Hz, 4H), 3.39-3.31 (m, 4H), 3.01 (t, J=7.4 Hz, 2H), 2.40-2.27 (m, 6H), 2.14-2.02 (m, 2H), 1.77-1.60 (m, 2H). LCMS^{C} m/z=405 [M+H]⁺

### Example 130: 6-chloro-5,7-dimethyl-N²-(1-methylpiperidin-4-yl)pyrido[2,3-d]pyrimidine-2,4-diamine

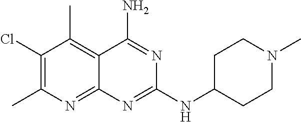

A solution of Example 5 (100 mg, 0.349 mmol) and 1-chloropyrrolidine-2,5-dione (93.3 mg, 0.698 mmol) in THF (1.0 mL) and MeOH (1.0 mL) was stirred at room temperature for 1 h. The resulting mixture was concentrated under reduced pressure. The residue was dissolved in water (5 mL) and extracted with EtOAc (3×10 mL). The combined organic phase was dried over anhydrous Na₂SO₄. After filtration, the filtrate was concentrated under reduced pressure. The residue was purified by Prep-HPLC^{A}N to afford the title compound (48.2 mg, 45.2%) as an off-white solid. ¹H NMR (400 MHz, DMSO-d₆) δ 6.88 (br, 2H), 6.55 (br, 1H), 3.83-3.68 (m, 1H), 2.80-2.69 (m, 5H), 2.53 (s, 3H), 2.16 (s, 3H), 2.00-1.87 (m, 2H), 1.86-1.74 (m, 2H), 1.59-1.44 (m, 2H). LCMS^{C} m/z=321 [M+H]⁺.

### Example 131: tert-butyl 4-((4-amino-5,7-dimethylpyrido[2,3-d]pyrimidin-2-yl)amino)azepane-1-carboxylate

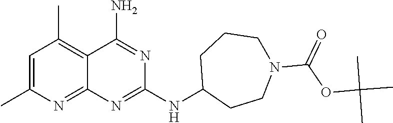 [SMILES: Cc1cc(C)c2c(N)nc(NC3CCCN(C(=O)OC(C)(C)C)CC3)nc2n1]

To a stirred mixture of 2-chloro-5,7-dimethylpyrido[2,3-d]pyrimidin-4-amine (60 mg, 0.288 mmol) in 1,4-dioxane (2 mL) at room temperature was added tert-butyl 4-aminoazepane-1-carboxylate (92.45 mg, 0.432 mmol) and CsF (130.46 mg, 0.864 mmol). The resulting mixture was stirred for 16 h at 110° C. The solution was cooled to room temperature and purified by Agela MP-Flash200^{Library} to afford the title compound as a solid (50 mg, 44.90%). LCMS^{Library A}: m/z=387 [M+H]⁺.

### Example 132-Example 139

The following Examples were synthesised in a similar manner to that described for Example 131, using the appropriate amine reagent:

| | | | LCMS |
|---|---|---|---|
| Example | Structure | ¹H NMR | m/z |
| Example 132 | 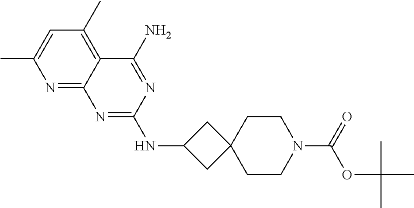 [SMILES: Cc1cc(C)c2c(N)nc(NC3CC4(CCN(C(=O)OC(C)(C)C)CC4)C3)nc2n1] | | Library A: 413 [M + H]+ |
| Example 133 | 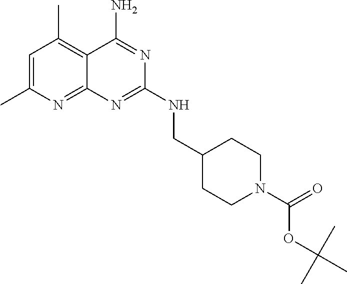 [SMILES: Cc1cc(C)c2c(N)nc(NCC3CCN(C(=O)OC(C)(C)C)CC3)nc2n1] | | Library A: 387 [M + H]+. |
| Example 134 | 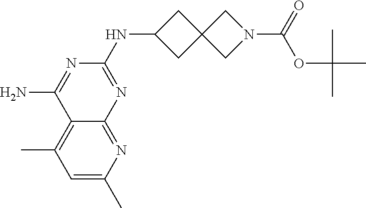 [SMILES: Cc1cc(C)c2c(N)nc(NC3CC4(C3)CN(C(=O)OC(C)(C)C)C4)nc2n1] | | Library A: 385 [M + H]+ |
| Example 135 | 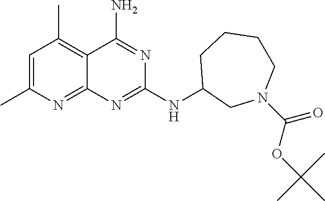 [SMILES: Cc1cc(C)c2c(N)nc(NC3CCCCN(C(=O)OC(C)(C)C)C3)nc2n1] | | Library A: 387 [M + H]+ |
| Example 136 | 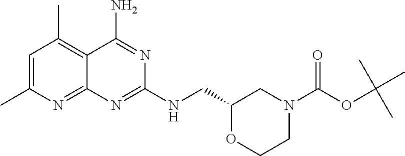 [SMILES: Cc1cc(C)c2c(N)nc(NC[C@@H]3CN(C(=O)OC(C)(C)C)CCO3)nc2n1] | | Library F: 389 [M + H]+ |
| Example 137 | 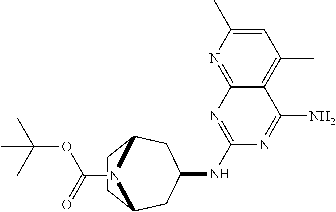 | | Library E: 399 [M + H]+ |
| | Mixture of cis isomers | | |
| Example 138 | 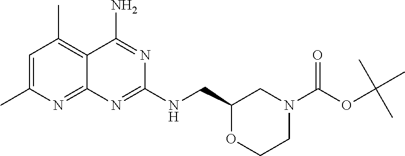 [SMILES: Cc1cc(C)c2c(N)nc(NC[C@H]3CN(C(=O)OC(C)(C)C)CCO3)nc2n1] | | Library F: 389 [M + H]+ |
| Example 139 | 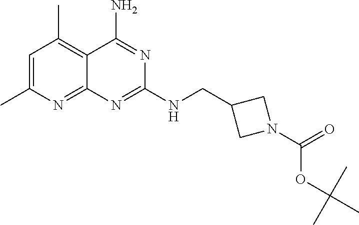 [SMILES: Cc1cc(C)c2c(N)nc(NCC3CN(C(=O)OC(C)(C)C)C3)nc2n1] | | Library A: 359 [M + H]+ |

### Example 140: A2-(azepan-4-yl)-5,7-dimethylpyrido[2,3-d]pyrimidine-2,4-diamine

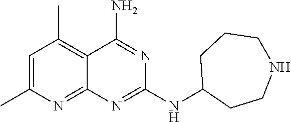 [SMILES: Cc1cc(C)c2c(N)nc(NC3CCCNCC3)nc2n1]

To a stirred mixture of Example 131 (50 mg, 0.129 mmol) in DCM (2 ml-) at room temperature was added 4.0 M HCl in 1,4-dioxane (0.5 mL). The resulting mixture was stirred for 2 h at room temperature. Removed the solvent under pressure to afford the title compound as a solid hydrochloride salt (22.8 mg, 54.88%). LCMS^{Library E}: m/z=287 [M+H]⁺.

### Example 141: 5,7-dimethyl-N²-(piperidin-4-ylmethyl)pyrido[2,3-d]pyrimidine-2,4-diamine

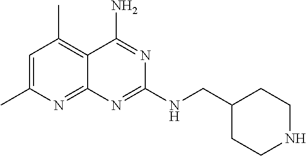 [SMILES: Cc1cc(C)c2c(N)nc(NCC3CCNCC3)nc2n1]

Synthesized in a similar manner to that described for Example 140, from Example 133. LCMS ^{Library B}: m/z=287 [M+H]⁺.

### Example 142: N²-(2-(dimethylamino)ethyl)-5,7-dimethylpyrido[2,3-d]pyrimidine-2,4-diamine

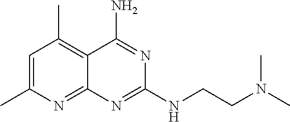

To a stirred mixture of 2-chloro-5,7-dimethylpyrido[2,3-d]pyrimidin-4-amine (50 mg, 0.240 mmol) in 1,4-Dioxane (2 mL) at room temperature was added N¹,N¹-dimethylethane-1,2-diamine (31.73 mg, 0.360 mmol) and CsF (108.72 mg, 0.72 mmol). The resulting mixture was stirred for 16 h at 110° C. The solution was cooled to room temperature and purified by Agela MP-Flash200^{Library} to afford the title compound as a solid (24.7 mg, 39.58%). LCMS^{Library A}: m/z=261 [M+H]⁺.

### Example 143-Example 218

The following Examples were synthesised in a similar manner to that described for Example 142, using the appropriate amine reagent:

| | | | LCMS |
|---|---|---|---|
| Example | Structure | ¹H NMR | m/z |
| Example 143 | 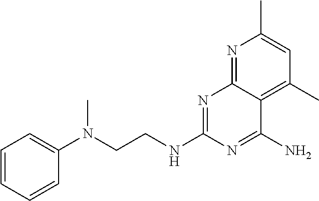 [SMILES: Cc1cc(C)c2c(N)nc(NCCN(C)c3ccccc3)nc2n1] | | Library A: 323 [M + H]+ |
| Example 144 | 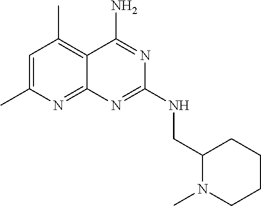 [SMILES: Cc1cc(C)c2c(N)nc(NCC3CCCCN3C)nc2n1] | | Library A: 301 [M + H]+ |
| Example 145 | 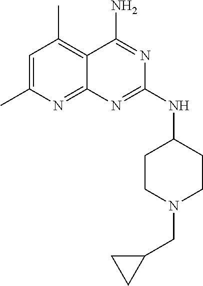 [SMILES: Cc1cc(C)c2c(N)nc(NC3CCN(CC4CC4)CC3)nc2n1] | | Library A: 327 [M + H]+ |
| Example 146 | 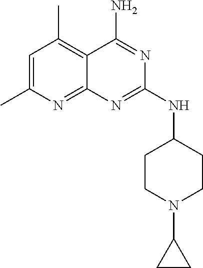 [SMILES: Cc1cc(C)c2c(N)nc(NC3CCN(C4CC4)CC3)nc2n1] | | Library A: 313 [M + H]+ |
| Example 147 | 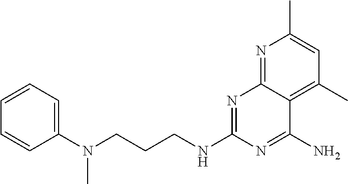 [SMILES: Cc1cc(C)c2c(N)nc(NCCCN(C)c3ccccc3)nc2n1] | | Library A: 337 [M + H]+ |
| Example 148 | 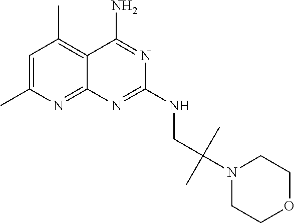 [SMILES: Cc1cc(C)c2c(N)nc(NCC(C)(C)N3CCOCC3)nc2n1] | | Library A: 331 [M + H]+ |
| Example 149 | 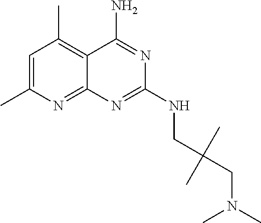 | | Library A: 303 [M + H]+ |
| Example 150 | 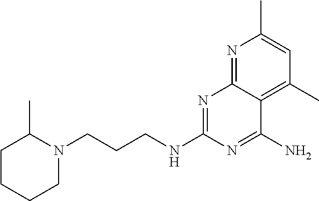 [SMILES: Cc1cc(C)c2c(N)nc(NCCCN3CCCCC3C)nc2n1] | | Library E: 329 [M + H]+ |
| Example 151 | 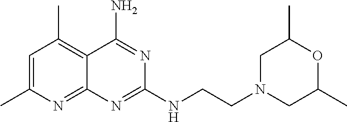 | | Library A: 331 [M + H]+ |
| Example 152 | 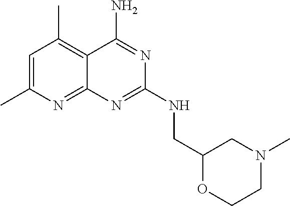 | | Library A: 303 [M + H]+ |
| Example 153 | 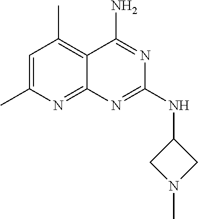 [SMILES: Cc1cc(C)c2c(N)nc(NC3CN(C)C3)nc2n1] | | Library F: 259 [M + H]+ |
| Example 154 | 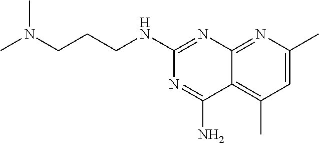 | | Library A: 275 [M + H]+ |
| Example 155 | 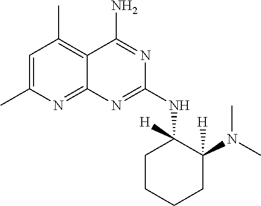 | | Library E: 315 [M + H]+ |
| Example 156 | 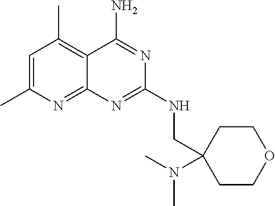 [SMILES: Cc1cc(C)c2c(N)nc(NCC3(N(C)C)CCOCC3)nc2n1] | | Library A: 331 [M + H]+ |
| Example 157 | 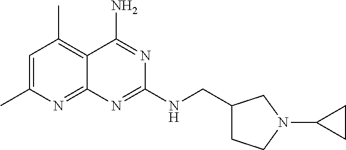 [SMILES: Cc1cc(C)c2c(N)nc(NCC3CCN(C4CC4)C3)nc2n1] | | Library A: 313 [M + H]+ |
| Example 158 | 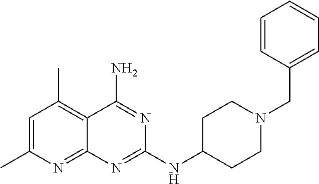 [SMILES: Cc1cc(C)c2c(N)nc(NC3CCN(Cc4ccccc4)CC3)nc2n1] | | Library A: 363 [M + H]+ |
| Example 159 | 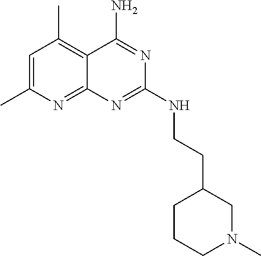 | | Library B: 315 [M + H]+ |
| Example 160 | 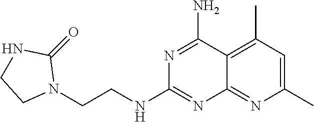 [SMILES: Cc1cc(C)c2c(N)nc(NCCN3CCNC3=O)nc2n1] | | Library A: 302 [M + H]+ |
| Example 161 | 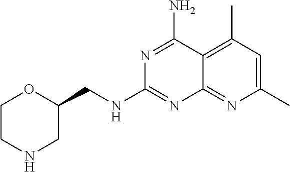 [SMILES: Cc1cc(C)c2c(N)nc(NC[C@H]3CNCCO3)nc2n1] | | Library A: 289 [M + H]+ |
| Example 162 | 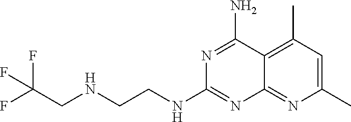 [SMILES: Cc1cc(C)c2c(N)nc(NCCNCC(F)(F)F)nc2n1] | | Library A: 315 [M + H]+ |
| Example 163 | 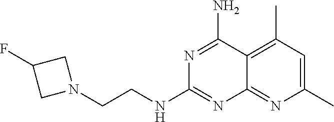 [SMILES: Cc1cc(C)c2c(N)nc(NCCN3CC(F)C3)nc2n1] | | Library A: 291 [M + H]+ |
| Example 164 | 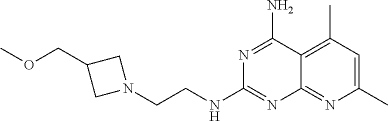 | | Library A: 317 [M + H]+ |
| Example 165 | 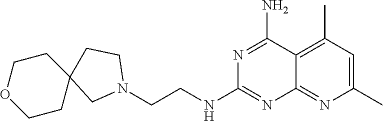 [SMILES: Cc1cc(C)c2c(N)nc(NCCN3CCC4(CCOCC4)C3)nc2n1] | | Library I: 357 [M + H]+ |
| Example 166 | 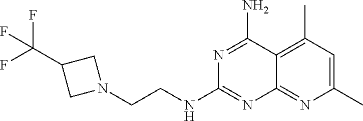 [SMILES: Cc1cc(C)c2c(N)nc(NCCN3CC(C(F)(F)F)C3)nc2n1] | | Library E: 341 [M + H]+ |
| Example 167 | 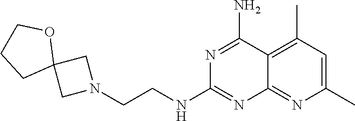 [SMILES: Cc1cc(C)c2c(N)nc(NCCN3CC4(CCCO4)C3)nc2n1] | | Library A: 329 [M + H]+ |
| Example 168 | 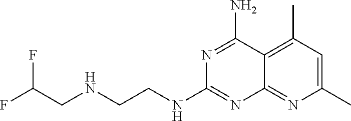 [SMILES: Cc1cc(C)c2c(N)nc(NCCNCC(F)F)nc2n1] | | Library A: 297 [M + H]+ |
| Example 169 | 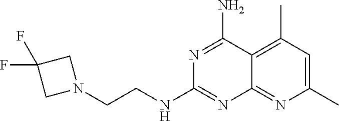 [SMILES: Cc1cc(C)c2c(N)nc(NCCN3CC(F)(F)C3)nc2n1] | | Library A: 309 [M + H]+ |
| Example 170 | 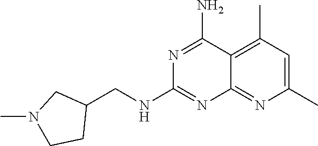 | | Library E: 287 [M + H]+ |
| Example 171 | 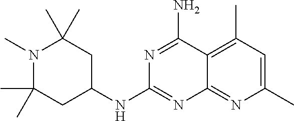 | | Library E: 343 [M + H]+ |
| Example 172 | 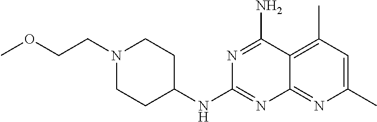 | | Library A: 331 [M + H]+ |
| Example 173 | 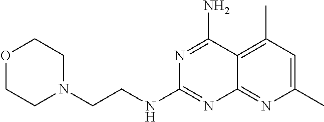 [SMILES: Cc1cc(C)c2c(N)nc(NCCN3CCOCC3)nc2n1] | | Library A: 303 [M + H]+ |
| Example 174 | 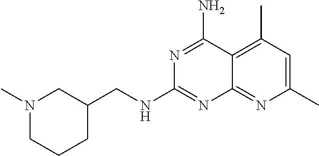 | | Library E: 301 [M + H]+ |
| Example 175 | 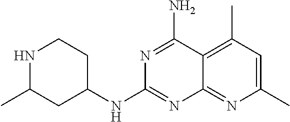 | | Library E: 287 [M + H]+ |
| Example 176 | 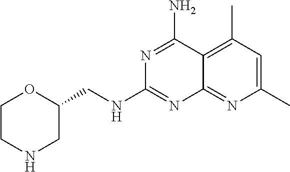 [SMILES: Cc1cc(C)c2c(N)nc(NC[C@@H]3CNCCO3)nc2n1] | | Library A: 289 [M + H]+ |
| Example 177 | 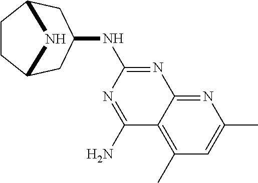 | | Library E: 299 [M + H]+ |
| | Mixture of cis isomers | | |
| Example 178 | 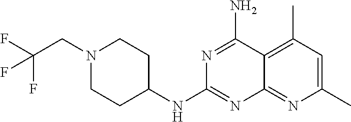 [SMILES: Cc1cc(C)c2c(N)nc(NC3CCN(CC(F)(F)F)CC3)nc2n1] | | Library A: 355 [M + H]+ |
| Example 179 | 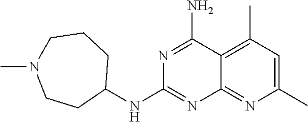 | | Library E: 301 [M + H]+ |
| Example 180 | 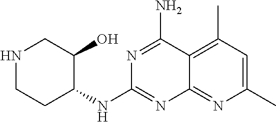 [SMILES: Cc1cc(C)c2c(N)nc(N[C@@H]3CCNC[C@H]3O)nc2n1] | ¹H NMR (400 MHz, DMSO- d₆) δ 6.78 (s, 2H), 6.70 (s, 1H), 6.39 (d, J = 6.8 Hz, 1H), 5.50 (s, 1H), 3.76-3.58 (m, 1H), 3.30-3.26 (m, 1H), 3.00 (dd, J = 12.1, 4.6 Hz, 1H), 2.85 (d, J = 12.5 Hz, 1H), 2.64 (s, 3H), 2.47-2.40 (m, 1H), 2.38 (s, 3H), 2.31- 2.21 (m, 1H), 1.95-1.84 (m, 1H), 1.41-1.26 (m, 1H). | Library A: 289 [M + H]+ |
| Example 181 | 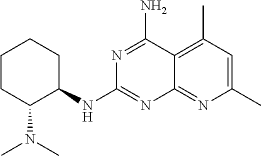 [SMILES: Cc1cc(C)c2c(N)nc(N[C@@H]3CCCC[C@H]3N(C)C)nc2n1] | | Library E: 315 [M + H]+ |
| Example 182 | 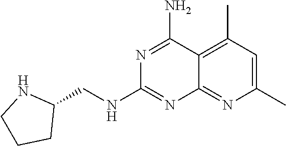 [SMILES: Cc1cc(C)c2c(N)nc(NC[C@@H]3CCCN3)nc2n1] | | Library E: 273 [M + H]+ |
| Example 183 | 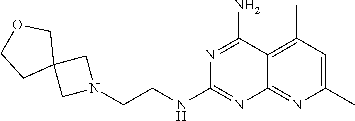 [SMILES: Cc1cc(C)c2c(N)nc(NCCN3CC4(CCOC4)C3)nc2n1] | | Library A: 329 [M + H]+ |
| Example 184 | 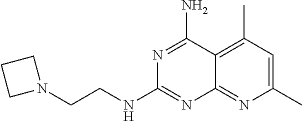 [SMILES: Cc1cc(C)c2c(N)nc(NCCN3CCC3)nc2n1] | | Library J: 273 [M + H]+ |
| Example 185 | 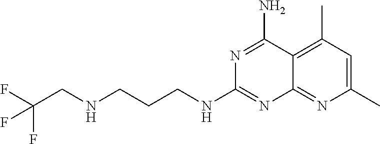 [SMILES: Cc1cc(C)c2c(N)nc(NCCCNCC(F)(F)F)nc2n1] | | Library A: 329 [M + H]+ |
| Example 186 | 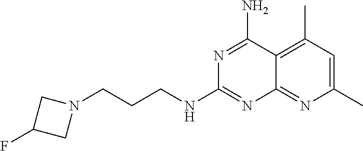 [SMILES: Cc1cc(C)c2c(N)nc(NCCCN3CC(F)C3)nc2n1] | | Library A: 305 [M + H]+ |
| Example 187 | 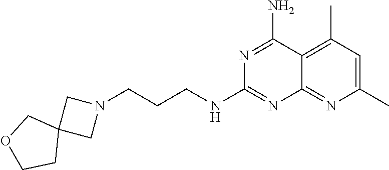 [SMILES: Cc1cc(C)c2c(N)nc(NCCCN3CC4(CCOC4)C3)nc2n1] | | Library A: 343 [M + H]+ |
| Example 188 | 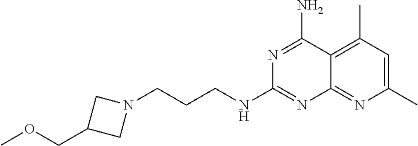 | | Library E: 331 [M + H]+ |
| Example 189 | 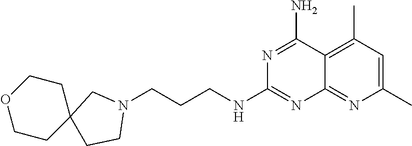 [SMILES: Cc1cc(C)c2c(N)nc(NCCCN3CCC4(CCOCC4)C3)nc2n1] | | Library E: 371 [M + H]+ |
| Example 190 | 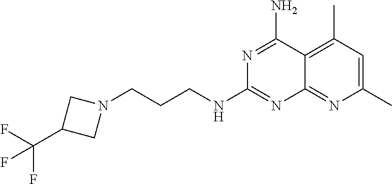 [SMILES: Cc1cc(C)c2c(N)nc(NCCCN3CC(C(F)(F)F)C3)nc2n1] | | Library A: 355 [M + H]+ |
| Example 191 | 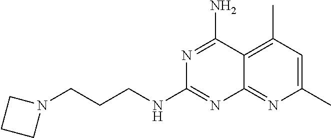 [SMILES: Cc1cc(C)c2c(N)nc(NCCCN3CCC3)nc2n1] | | Library B: 287 [M + H]+ |
| Example 192 | 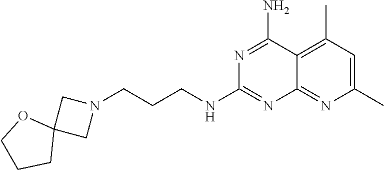 [SMILES: Cc1cc(C)c2c(N)nc(NCCCN3CC4(CCCO4)C3)nc2n1] | | Library E: 343 [M + H]+ |
| Example 193 | 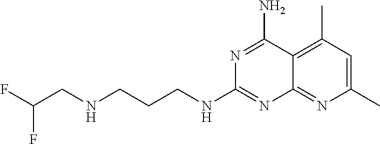 [SMILES: Cc1cc(C)c2c(N)nc(NCCCNCC(F)F)nc2n1] | | Library A: 311 [M + H]+ |
| Example 194 | 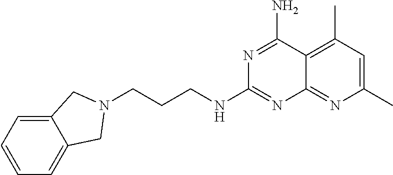 [SMILES: Cc1cc(C)c2c(N)nc(NCCCN3Cc4ccccc4C3)nc2n1] | | Library A: 349 [M + H]+ |
| Example 195 | 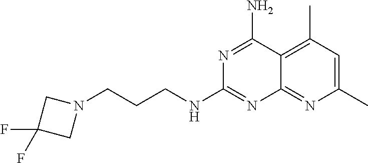 [SMILES: Cc1cc(C)c2c(N)nc(NCCCN3CC(F)(F)C3)nc2n1] | | Library A: 323 [M + H]+ |
| Example 196 | 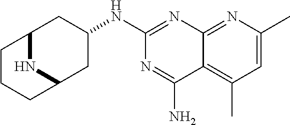 | | Library E: 313 [M + H]+ |
| | Mixture of trans isomers | | |
| Example 197 | 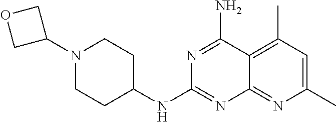 [SMILES: Cc1cc(C)c2c(N)nc(NC3CCN(C4COC4)CC3)nc2n1] | | Library A: 329 [M + H]+ |
| Example 198 | 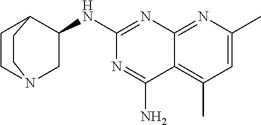 [SMILES: Cc1cc(C)c2c(N)nc(N[C@H]3CN4CCC3CC4)nc2n1] | | Library I: 299 [M + H]+ |
| Example 199 | 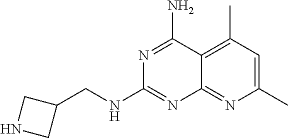 [SMILES: Cc1cc(C)c2c(N)nc(NCC3CNC3)nc2n1] | | Library E: 259 [M + H]+ |
| Example 200 | 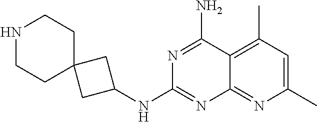 [SMILES: Cc1cc(C)c2c(N)nc(NC3CC4(CCNCC4)C3)nc2n1] | | Library I: 313 [M + H]+ |
| Example 201 | 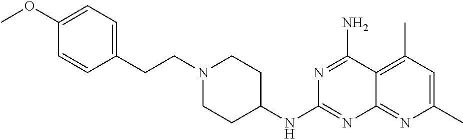 | | Library A: 407 [M + H]+ |
| Example 202 | 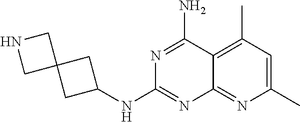 [SMILES: Cc1cc(C)c2c(N)nc(NC3CC4(CNC4)C3)nc2n1] | | Library E: 285 [M + H]+ |
| Example 203 | 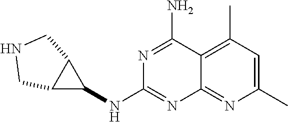 | | Library I: 271 [M + H]+ |
| | Mixture of trans isomers | | |
| Example 204 | 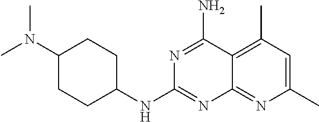 | | Library E: 315 [M + H]+ |
| Example 205 | 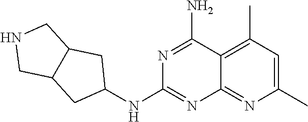 [SMILES: Cc1cc(C)c2c(N)nc(NC3CC4CNCC4C3)nc2n1] | | Library E: 299 [M + H]+ |
| Example 206 | 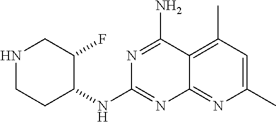 | | Library A: 291 [M + H]+ |
| | Mixture of cis isomers | | |
| Example 207 | 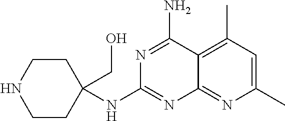 [SMILES: Cc1cc(C)c2c(N)nc(NC3(CO)CCNCC3)nc2n1] | | Library I: 303 [M + H]+ |
| Example 208 | 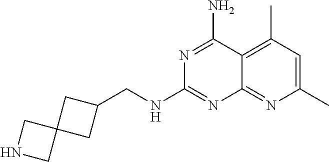 [SMILES: Cc1cc(C)c2c(N)nc(NCC3CC4(CNC4)C3)nc2n1] | | Library E: 299 [M + H]+ |
| Example 209 | 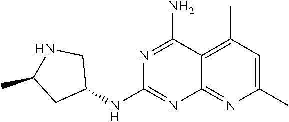 | | Library B: 273 [M + H]+ |
| Example 210 | 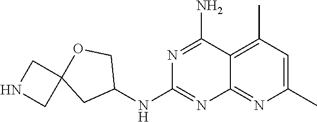 [SMILES: Cc1cc(C)c2c(N)nc(NC3COC4(CNC4)C3)nc2n1] | | Library E: 301 [M + H]+ |
| Example 211 | 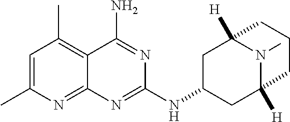 | | Library E: 327 [M + H]+ |
| | Mixture of cis isomers | | |
| Example 212 | 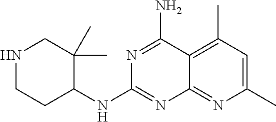 [SMILES: Cc1cc(C)c2c(N)nc(NC3CCNCC3(C)C)nc2n1] | | Library B: 301 [M + H]+ |
| Example 213 | 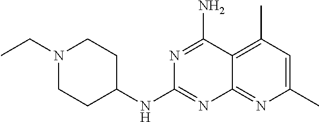 [SMILES: CCN1CCC(Nc2nc(N)c3c(C)cc(C)nc3n2)CC1] | | Library I: 301 [M + H]+ |
| Example 214 | 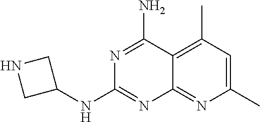 [SMILES: Cc1cc(C)c2c(N)nc(NC3CNC3)nc2n1] | | Library E: 245 [M + H]+ |
| Example 215 | 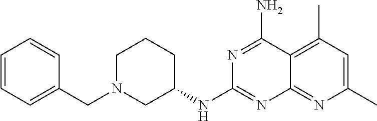 [SMILES: Cc1cc(C)c2c(N)nc(N[C@H]3CCCN(Cc4ccccc4)C3)nc2n1] | | Library A: 363 [M + H]+ |
| Example 216 | 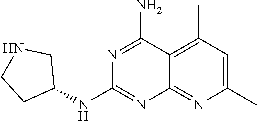 [SMILES: Cc1cc(C)c2c(N)nc(N[C@@H]3CCNC3)nc2n1] | ¹H NMR (400 MHz, DMSO- d₆) δ 6.96-6.38 (m, 4H), 4.41-4.31 (m, 1H), 3.62- 3.52 (m, 1 H), 3.02-2.86 (m, 2H), 2.80-2.69 (m, 1H), 2.64 (s, 3H), 2.64-2.59 (m, 1H), 2.38 (s, 3H), 2.03-1.90 (m, 1H), 1.68-1.50 (m, 1H) | Library B: 259 [M + H]+ |
| Example 217 | 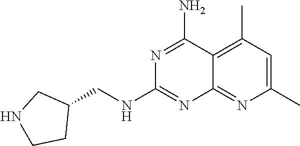 [SMILES: Cc1cc(C)c2c(N)nc(NC[C@@H]3CCNC3)nc2n1] | | Library E: 273 [M + H]+ |
| Example 218 | 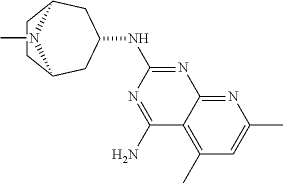 | | Library C: 313 [M + H]+ |
| | Mixture of cis isomers | | |

### Example 219: N²-(1-((dimethylamino)methyl)cyclopropyl)-5,7-dimethylpyrido[2,3-d]pyrimidine-2,4-diamine

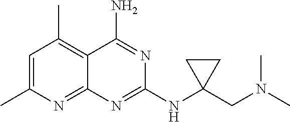

To a stirred mixture of 2-chloro-5,7-dimethylpyrido[2,3-d]pyrimidin-4-amine (60 mg, 0.29 mmol) in ethyl alcohol (1 mL) at room temperature was added 1-((dimethylamino)methyl)cyclopropan-1-amine (98.7 mg, 0.86 mmol). The solvent was removed under reduced pressure and then the resulting neat mixture was stirred for 6-12 h at 110° C. The solution was cooled to room temperature and purified by Agela MP-Flash200^{Library} to afford the title compound as a solid (3.7 mg, 4.5%). LCMS^{Library B}: m/z=287 [M+H]⁺.

### Example 220-Example 222

The following Examples were synthesised in a similar manner to that described for Example 219, using the appropriate amine reagent:

| | | | LCMS |
|---|---|---|---|
| Example | Structure | ¹H NMR | m/z |
| Example 220 | 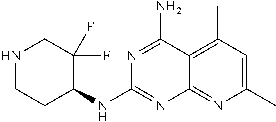 [SMILES: Cc1cc(C)c2c(N)nc(N[C@H]3CCNCC3(F)F)nc2n1] | | Library C: 309 [M + H]+ |
| Example 221 | 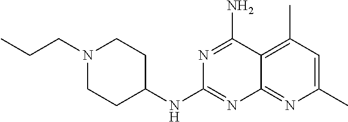 [SMILES: CCCN1CCC(Nc2nc(N)c3c(C)cc(C)nc3n2)CC1] | | Library A: 315 [M + H]+ |
| Example 222 | 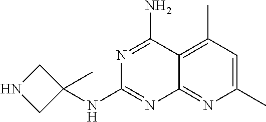 [SMILES: Cc1cc(C)c2c(N)nc(NC3(C)CNC3)nc2n1] | | Library C: 259 [M + H]+ |

### Example 223: (R)—N²-(1-(2-methoxyethyl)pyrrolidin-3-yl)-5,7-dimethylpyrido[2,3-d]pyrimidine-2,4-diamine

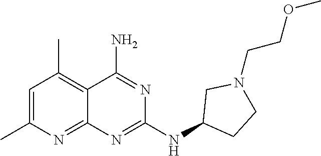

### Preparation 101—tert-butyl (R)-(1-(2-methoxyethyl)pyrrolidin-3-yl)carbamate

To a stirred solution of tert-butyl (R)-pyrrolidin-3-ylcarbamate (500 mg, 2.68 mmol) and 1-bromo-2-methoxyethane (448 mg, 3.22 mmol) in ACN (75.0 mL) were added K₂CO₃ (1.86 g, 13.4 mmol) and KI (446 mg, 2.68 mmol) at room temperature. The resulting mixture was stirred at 80° C. overnight. The mixture was allowed to cool down to room temperature. The resulting mixture was filtered, the filter cake was washed with ACN (3×10 mL). The filtrate was concentrated under reduced pressure. The residue was purified by silica gel column chromatography (EA:MeOH=10:1) to afford the title compound as a yellow oil (520 mg, 79.3%). LCMS m/z=245 [M+H]⁺

### Preparation 102—(R)-1-(2-methoxyethyl)pyrrolidin-3-amine hydrogen chloride

A mixture of tert-butyl (R)-(1-(2-methoxyethyl)pyrrolidin-3-yl)carbamate (520 mg, 2.13 mmol) and 4.0 M HCl in 1,4-dioxane (4.0 mL) was stirred at room temperature for 1 h. The resulting mixture was concentrated under reduced pressure. This resulted in the title compound as a yellow oil (470 mg, crude). LCMS m/z=145 [M+H]⁺

### Preparation 103—(R)—N²-(1-(2-methoxyethyl)pyrrolidin-3-yl)-5,7-dimethylpyrido[2,3-d]pyrimidine-2,4-diamine

To a stirred solution of (R)-1-(2-methoxyethyl)pyrrolidin-3-amine hydrogen chloride (458 mg, 2.54 mmol) and 2-chloro-5,7-dimethylpyrido[2,3-d]pyrimidin-4-amine (230 mg, 1.10 mmol) in dioxane (8.00 mL) was added Et₃N (919 μL, 6.61 mmol) at room temperature. The resulting mixture was stirred at 100° C. overnight. The mixture was allowed to cool down to room temperature. The resulting mixture was concentrated under reduced pressure. The residue was purified by Prep-HPLC^{AV} to afford the title compound as a yellow solid (170 mg, 16.2%, ee>99%). ¹H NMR (400 MHz, Methanol-d₄) δ 6.79 (s, 1H), 4.67-4.57 (m, 1H), 3.53 (t, J=5.6 Hz, 2H), 3.34 (s, 3H), 3.01-2.80 (m, 2H), 2.75-2.67 (m, 5H), 2.65-2.52 (m, 2H), 2.50-2.43 (m, 3H), 2.39-2.29 (m, 1H), 1.80-1.62 (m, 1H). LCMS^{R} m/z=317 [M+H]⁺

### Example 224: (R)-2-(3-((4-amino-5,7-dimethylpyrido[2,3-d]pyrimidin-2-yl)amino)pyrrolidin-1-yl)ethan-1-ol

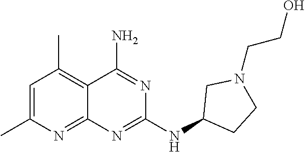 [SMILES: Cc1cc(C)c2c(N)nc(N[C@@H]3CCN(CCO)C3)nc2n1]

A mixture of Example 223 (145 mg, 0.458 mmol) and 1 M BBr₃ in DCM (3.0 mL) was stirred at room temperature for 1 h. The reaction was quenched with water/ice at 0° C. The resulting mixture was concentrated under reduced pressure. The residue was purified by Prep-HPLC^{V} to afford the title compound as a white solid (27.9 mg, 20.1%, ee>99%). ¹H NMR (400 MHz, Methanol-d₄) δ 6.80 (s, 1H), 4.68-4.55 (m, 1H), 3.68 (t, J=6.1 Hz, 2H), 3.05-2.83 (m, 2H), 2.71 (s, 3H), 2.69-2.52 (m, 4H), 2.47 (s, 3H), 2.41-2.30 (m, 1H), 1.78-1.65 (m, 1H). LCMS^{S} m/z=303 [M+H]⁺

### Example 225: (R)-5,7-dimethyl-N²-(piperidin-3-yl)pyrido[2,3-d]pyrimidine-2,4-diamine

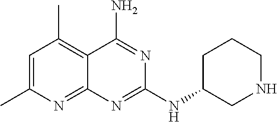 [SMILES: Cc1cc(C)c2c(N)nc(N[C@@H]3CCCNC3)nc2n1]

### Preparation 104—tert-butyl (R)-3-((4-amino-5,7-dimethylpyrido[2,3-d]pyrimidin-2-yl)amino)piperidine-1-carboxylate

A mixture of 2-chloro-5,7-dimethylpyrido[2,3-d]pyrimidin-4-amine (300 mg, 1.43 mmol) and tert-butyl (R)-3-aminopiperidine-1-carboxylate (345 mg, 1.72 mmol) in DMSO (5.00 mL) was stirred at 120° C. overnight. The mixture was allowed to cool down to room temperature. The residue was purified by Prep-HPLC^{AW} to afford the title compound as a yellow oil (340 mg, 63.4%). LCMS m/z=373 [M+H]⁺.

### Preparation 105—(R)-5,7-dimethyl-N²-(piperidin-3-yl)pyrido[2,3-d]pyrimidine-2,4-diamine

A mixture of tert-butyl (R)-3-((4-amino-5,7-dimethylpyrido[2,3-d]pyrimidin-2-yl)amino)piperidine-1-carboxylate (340 mg, 0.913 mmol) and HCl in dioxane (5.00 M, 4.0 M) was stirred at room temperature for 20 min. The resulting mixture was concentrated under reduced pressure. The residue was diluted with MeOH and basified to pH 7 with NH₃·H₂O. The residue was purified by Prep-HPLC^{L} to afford the title compound as a white solid (24.2 mg, 9.70%, ee>99%). ¹H NMR (400 MHz, DMSO-d₆) δ 6.89-6.55 (m, 3H), 6.25 (s, 1H), 3.91-3.79 (m, 1H), 3.23-3.11 (m, 1H), 3.00 (d, J=11.4 Hz, 1H), 2.76 (d, J=12.4 Hz, 1H), 2.63 (s, 3H), 2.48-2.30 (m, 5H), 1.89-1.74 (m, 1H), 1.69-1.55 (m, 1H), 1.49-1.33 (m, 2H). LCMS^{S} m/z=273 [M+H]⁺.

### Example 226: (3S,4S)-4-((4-amino-5,7-dimethylpyrido[2,3-d]pyrimidin-2-yl)amino)-1-methylpyrrolidin-3-ol

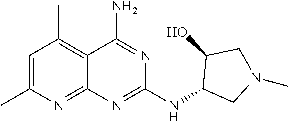

### Preparation 106—tert-butyl (3S,4S)-3-((4-amino-5,7-dimethylpyrido[2,3-d]pyrimidin-2-yl)amino)-4-hydroxypyrrolidine-1-carboxylate

To a stirred solution of 2-chloro-5,7-dimethylpyrido[2,3-d]pyrimidin-4-amine (1.00 g, 4.79 mmol) in dioxane (15.0 mL) at room temperature was added tert-butyl (3S,4S)-3-amino-4-hydroxypyrrolidine-1-carboxylate (1.94 g, 9.60 mmol, 2.00 equiv). The resulting mixture was stirred at 60° C. for 4 h. The mixture was allowed to cool down to room temperature. The precipitated solids were collected by filtration and washed with EtOAc (3×10 mL). This resulted in the title compound as an off-white solid (1.40 g, crude). LCMS m/z=375 [M+H]⁺.

### Preparation 107—(3S,4S)-4-((4-amino-5,7-dimethylpyrido[2,3-d]pyrimidin-2-yl)amino)-1-methylpyrrolidin-3-ol

To a stirred solution of tert-butyl (3S,4S)-3-((4-amino-5,7-dimethylpyrido[2,3-d]pyrimidin-2-ylamino)-4-hydroxypyrrolidine-1-carboxylate (1.00 g, 2.67 mmol) in THF (25 mL) was added 1 M LiAlH₄ in THF (21.4 mL) dropwise at 0° C. The resulting mixture was stirred at room temperature overnight. The reaction was quenched with methanol at 0° C. The resulting mixture was concentrated under reduced pressure. The residue was purified by Prep-HPLC^{A}S to afford the title compound as a white solid (54.1 mg, 6.7%, ee>99%). ¹H NMR (400 MHz, DMSO-d₆) δ 6.96-6.78 (m, 3H), 6.75 (s, 1H), 6.02 (s, 1H), 4.08-3.82 (m, 2H), 2.85 (t, J=8.3 Hz, 1H), 2.76-2.68 (m, 1H), 2.66 (s, 3H), 2.48-2.40 (m, 2H), 2.39 (s, 3H), 2.21 (s, 3H). LCMS^{T} m/z=289 [M+H]⁺.

### Example 227: N²-((3S,4S)-4-methoxypyrrolidin-3-yl)-N⁴,5,7-trimethylpyrido[2,3-d]pyrimidine-2,4-diamine

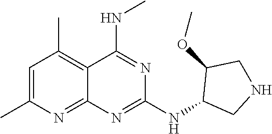 [SMILES: CNc1nc(N[C@H]2CNC[C@@H]2OC)nc2nc(C)cc(C)c12]

### Preparation 108—2-chloro-N,5,7-trimethylpyrido[2,3-d]pyrimidin-4-amine

To a stirred mixture of 2,4-dichloro-5,7-dimethylpyrido[2,3-d]pyrimidine (3.00 g, 13.2 mmol) and Et₃N (2.66 g, 26.3 mmol) in THE (60.0 mL) was added methanamine hydrochloride (1.72 g, 26.3 mmol) in portions at 0° C. The resulting mixture was stirred at room temperature for 3 h. The resulting mixture was concentrated under reduced pressure. The residue was diluted with water (100 mL). The precipitated solids were collected by filtration and washed with water (3×10 mL). This result in the title compound (1.60 g, 54.6%) as a pink solid. LCMS m/z=223 [M+H]⁺.

### Preparation 109—tert-butyl (3S,4S)-3-((5,7-dimethyl-4-(methylamino)pyrido[2,3-d]pyrimidin-2-yl)amino)-4-methoxypyrrolidine-1-carboxylate

To a solution of 2-chloro-N,5,7-trimethylpyrido[2,3-d]pyrimidin-4-amine (200 mg, 0.89 mmol) in 1,4-dioxane (2.00 mL) was added tert-butyl (3S,4S)-3-amino-4-methoxypyrrolidine-1-carboxylate (233 mg, 1.07 mmol). The resulting mixture was stirred at 100° C. overnight. Then, the resulting mixture was allowed to cool down to room temperature and concentrated under reduced pressure. The residue was purified by Prep-HPLC^{A}V to afford the title compound (362 mg, crude) as an off-white solid. LCMS m/z=403 [M+H]⁺.

### Preparation 110—N²-((3S,4S)-4-methoxypyrrolidin-3-yl)-N⁴,5,7-trimethylpyrido[2,3-d]pyrimidine-2,4-diamine

To a solution of tert-butyl (3S,4S)-3-([5,7-dimethyl-4-(methylamino)pyrido[2,3-d]pyrimidin-2-yl]amino-4-methoxypyrrolidine-1-carboxylate (340 mg, 0.840 mmol) in 1,4-dioxane (3.40 mL) was added 4 M HCl in MeOH (3.00 mL). The resulting mixture was stirred at room temperature for 1 h and concentrated under reduced pressure. The residue was dissolved in MeOH (2.00 mL) and basified to pH 9 with Et₃N. The residue was purified by Prep-HPLC^{AV} to afford the title compound (130.8 mg, 50.3%, ee>99%) as an off-white solid. ¹H NMR (400 MHz, DMSO-d₆) δ 6.96-6.72 (m, 2H), 6.69 (s, 1H), 4.36-4.25 (m, 1H), 3.76-3.71 (m, 1H), 3.32 (s, 3H), 3.12-3.00 (m, 2H), 2.95 (s, 3H), 2.71-2.62 (m, 5H), 2.38 (s, 3H). LCMS^{U} m/z=303 [M+H]⁺.

### Example 228: N²-((3S,4S)-4-methoxy-1-methylpyrrolidin-3-yl)-N⁴,5,7-trimethylpyrido[2,3-d]pyrimidine-2,4-diamine

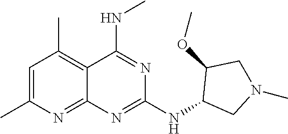

A solution of Example 227 (95.0 mg, 0.314 mmol) in DCM (1.00 mL) was treated with paraformaldehyde (47.1 mg, 1.57 mmol) for 30 min at room temperature followed by the addition of STAB (532 mg, 2.51 mmol) in portions at room temperature. The resulting mixture was stirred at room temperature overnight. The reaction was quenched by the addition of water (2 mL). The solid was filtered out, the filtrate was concentrated under reduced pressure. The residue was purified by Prep-HPLC^{AV} to afford the title compound (21.7 mg, 21.0%, ee>99%) as an off-white solid. ¹H NMR (400 MHz, DMSO-d₆) δ 6.95-6.59 (m, 3H), 4.40-4.274 (m, 1H), 3.89-3.77 (m, 1H), 3.25 (s, 3H), 3.00-2.83 (m, 4H), 2.73-2.59 (m, 4H), 2.49-2.43 (m, 1H), 2.37 (s, 3H), 2.27-2.16 (m, 4H). LCMS^{U} m/z=317 [M+H]⁺.

### Example 229: N²-(1-benzylpiperidin-4-yl)-5-methyl-7,8-dihydro-6H-cyclopenta[5,6]pyrido[2,3-d]pyrimidine-2,4-diamine

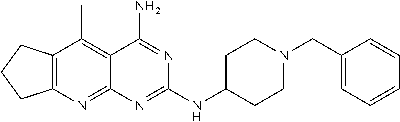 [SMILES: Cc1c2c(nc3nc(NC4CCN(Cc5ccccc5)CC4)nc(N)c13)CCC2]

To a stirred mixture of N-(3-cyano-4-methyl-6,7-dihydro-5H-cyclopenta[b]pyridin-2-yl)cyanamide (100 mg, 0.504 mmol) in EtOH (5.0 mL) at room temperature was added 1-benzylpiperidin-4-amine (192 mg, 1.01 mmol). The resulting mixture was stirred at 100° C. for 1 h. The resulting mixture was concentrated under reduced pressure. The residue was purified by Prep-HPLC^{L} to afford the title compound as a white solid (24.7 mg, 12.6%). ¹H NMR (300 MHz, DMSO-d₆) δ 7.38-7.19 (m, 5H), 6.64 (s, 2H), 6.30 (s, 1H), 3.89-3.70 (m, 1H), 3.46 (s, 2H), 2.94-2.73 (m, 6H), 2.57 (s, 3H), 2.10-1.93 (m, 4H), 1.87-1.73 (m, 2H), 1.57-1.39 (m, 2H). LCMS^{V} m/z=389 [M+H]⁺.

### Example 230: (R)-5-methyl-N2-(pyrrolidin-3-yl)-7,8-dihydro-6H-cyclopenta[5,6]pyrido[2,3-d]pyrimidine-2,4-diamine

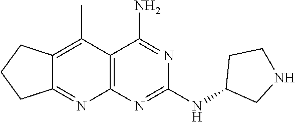 [SMILES: Cc1c2c(nc3nc(N[C@@H]4CCNC4)nc(N)c13)CCC2]

### Preparation 111—mixture of ethyl 3-amino-1-methyl-6,7-dihydro-5H-cyclopenta[c]pyridine-4-carboxylate and ethyl 2-amino-4-methyl-6,7-dihydro-5H-cyclopenta[b]pyridine-3-carboxylate

To a stirred mixture of ethyl 2-carbamimidoylacetate hydrochloride (66.0 g, 396 mmol) and piperidine (101 g, 1190 mmol) in EtOH (1.00 L) at room temperature was added acetylcyclopentanone (50.0 g, 396 mmol) dropwise. The resulting mixture was stirred at room temperature for 2 h. The resulting mixture was concentrated under reduced pressure. The residue was diluted with PE (100 mL). The precipitated solids were collected by filtration and purified by silica gel column with ethyl acetate/petroleum ether (3:1) to afford ethyl 3-amino-1-methyl-6,7-dihydro-5H-cyclopenta[c]pyridine-4-carboxylate (35.0 g, 40.1%) as white solid. The filtrate was concentrated under reduced pressure. The residue was purified by Prep-HPLC^{AW} to afford ethyl 2-amino-4-methyl-6,7-dihydro-5H-cyclopenta[b]pyridine-3-carboxylate (21.0 g, 24.0%) as off-white solid. LCMS m/z=221 [M+H]⁺.

### Preparation 112—ethyl 4-methyl-2-(3-(2,2,2-trichloroacetyl)ureido)-6,7-dihydro-5H-cyclopenta[b]pyridine-3-carboxylate

To a stirred solution of ethyl 2-amino-4-methyl-6,7-dihydro-5H-cyclopenta[b]pyridine-3-carboxylate (21.0 g, 95.0 mmol) in THF (210 mL) was added trichloroethanecarbonyl isocyanate (26.8 g, 142 mmol) dropwise at 0° C. The resulting mixture was stirred at room temperature for 1 h. The resulting mixture was concentrated under reduced pressure. The residue was purified by silica gel column with ethyl acetate/petroleum ether (3:1) to afford the title compound as white solid (38.0 g, 98.0%). LCMS m/z=408 [M+H]⁺.

### Preparation 113—5-methyl-7,8-dihydro-6H-cyclopenta[5,6]pyrido[2,3-d]pyrimidine-2,4-diol

Into a pressure tank reactor were added ethyl 1-methyl-3-(3-(2,2,2-trichloroacetyl)ureido)-6,7-dihydro-5H-cyclopenta[c]pyridine-4-carboxylate (38.0 g, 93.1 mmol) and 7.0 M NH₃ in MeOH (70.0 mL) in EtOH (460 mL) at room temperature. The resulting mixture was stirred at 80° C. overnight. The mixture was allowed to cool down to 0° C. The precipitated solids were collected by filtration and washed with EtOH (3×20 mL). This resulted in the title compound (19.2 g, 95.0%) as white solid. LCMS m/z=218 [M+H]⁺.

### Preparation 114—2,4-dichloro-5-methyl-7,8-dihydro-6H-cyclopenta[5,6]pyrido[2,3-d]pyrimidine

To a stirred mixture of 5-methyl-7,8-dihydro-6H-cyclopenta[5,6]pyrido[2,3-d]pyrimidine-2,4-diol (5.0 g, 2.30 mmol) and DIEA (20.0 mL, 11.5 mmol) in ACN (90.0 mL) was added POCl₃ (21.5 mL, 23.0 mmol) dropwise at 0° C. The resulting mixture was stirred at 60° C. for 2 h. The mixture was allowed to cool down to room temperature. To the above mixture was added 4 M HCl in 1,4-dioxane (25.0 mL) dropwise over 30 min at 0° C. The resulting mixture was stirred at room temperature for additional 2 h. The resulting mixture was concentrated under reduced pressure. The reaction was quenched by the addition of sat. NaHCO₃ (aq.) at 0° C.

The resulting mixture was extracted with DCM (3×100 mL). The combined organic layers were washed with brine (1×100 mL), dried over anhydrous Na₂SO₄. After filtration, the filtrate was concentrated under reduced pressure to afford the title compound (3.8 g, crude). The crude product was used in the next step directly without further purification. LCMS m/z=254 [M+H]⁺.

### Preparation 115—2-chloro-5-methyl-7,8-dihydro-6H-cyclopenta[5,6]pyrido[2,3-d]pyrimidin-4-amine

A mixture of 2,4-dichloro-5-methyl-7,8-dihydro-6H-cyclopenta[5,6]pyrido[2,3-d]pyrimidine (3.00 g, 11.8 mmol, 1.00 eq.) and NH₃·H₂O (60.0 mL) was stirred at room temperature for 2 days. The precipitated solids were collected by filtration and washed with water (3×30 mL). The solids were purified by trituration with ethyl acetate (30 mL). This resulted in the title compound (1.30 g, crude) as a black solid. LCMS m/z=235 [M+H]⁺.

### Preparation 116—tert-butyl (R)-3-((4-amino-5-methyl-7,8-dihydro-6H-cyclopenta[5,6]pyrido[2,3-d]pyrimidin-2-yl)amino)pyrrolidine-1-carboxylate

A mixture of 2-chloro-5-methyl-7,8-dihydro-6H-cyclopenta[5,6]pyrido[2,3-d]pyrimidin-4-amine (900 mg, 3.84 mmol) and tert-butyl (R)-3-aminopyrrolidine-1-carboxylate (2.86 g, 15.3 mmol) in dioxane (9.00 mL) was stirred at 100° C. overnight. The mixture was allowed to cool down to room temperature and concentrated under reduced pressure. The crude product (800 mg) as brown yellow was used in the next step directly without further purification. LCMS m/z=385 [M+H]⁺.

### Preparation 117—(R)-5-methyl-N²-(pyrrolidin-3-yl)-7,8-dihydro-6H-cyclopenta[5,6]pyrido[2,3-d]pyrimidine-2,4-diamine

A mixture of tert-butyl (R)-3-((4-amino-5-methyl-7,8-dihydro-6H-cyclopenta[5,6]pyrido[2,3-d]pyrimidin-2-yl)amino)pyrrolidine-1-carboxylate (800 mg, 2.08 mmol) and 4.0 M HCl in 1,4-dioxane (8.00 mL) was stirred at room temperature for 1 h. The resulting mixture was concentrated under reduced pressure and diluted with MeOH (8.00 mL). The residue was basified to pH 9 with TEA. The residue was purified by Prep-HPLC^{AV} to afford the title compound (377.8 mg, 63.8%, ee>99%) as light orange solid. ¹H NMR (400 MHz, DMSO-d₆) δ 6.90-6.32 (m, 3H), 4.44-4.27 (m, 1H), 2.98-2.79 (m, 6H), 2.79-2.69 (m, 1H), 2.64 (dd, J=11.2, 4.6 Hz, 1H), 2.58 (s, 3H), 2.14-1.92 (m, 3H), 1.68-1.54 (m, 1H). LCMS^{W} m/z=285 [M+H]⁺.

### Example 231: (R)—N⁴,5-dimethyl-N²-(pyrrolidin-3-yl)-7,8-dihydro-6H-cyclopenta[5,6]pyrido[2,3-d]pyrimidine-2,4-diamine

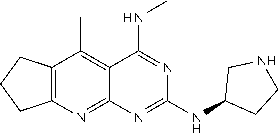 [SMILES: CNc1nc(N[C@@H]2CCNC2)nc2nc3c(c(C)c12)CCC3]

### Preparation 118—2-chloro-N,5-dimethyl-7,8-dihydro-6H-cyclopenta[5,6]pyrido[2,3-d]pyrimidin-4-amine

A mixture of 2,4-dichloro-5-methyl-7,8-dihydro-6H-cyclopenta[5,6]pyrido[2,3-d]pyrimidine (1.00 g, crude) and methylamine (2.0 M in THF, 10.0 mL) was stirred at room temperature for 1 h. The resulting mixture was concentrated under reduced pressure. The residue was purified by Prep-HPLC^{AV} to afford the title compound as brown solid (210 mg, 18.5% for two steps). LCMS m/z=249 [M+H]⁺.

### Preparation 119—tert-butyl (R)-3-((5-methyl-4-(methylamino)-7,8-dihydro-6H-cyclopenta[5,6]pyrido[2,3-d]pyrimidin-2-yl)amino)pyrrolidine-1-carboxylate

A mixture of 2-chloro-N,5-dimethyl-7,8-dihydro-6H-cyclopenta[5,6]pyrido[2,3-d]pyrimidin-4-amine (300 mg, 1.20 mmol) and tert-butyl (R)-3-aminopyrrolidine-1-carboxylate (447 mg, 2.40 mmol) in 1,4-dioxane (6.0 mL) was stirred at 100° C. for 1 h. The mixture was allowed to cool down to room temperature. The residue was purified by Prep-HPLC^{B} to afford the title compound (200 mg, 41.6%) as yellow oil. LCMS m/z=399 [M+H]⁺.

### Preparation 120—(R)—N4,5-dimethyl-N²-(pyrrolidin-3-yl)-7,8-dihydro-6H-cyclopenta[5,6]pyrido[2,3-d]pyrimidine-2,4-diamine

A mixture of tert-butyl (R)-3-((5-methyl-4-(methylamino)-7,8-dihydro-6H-cyclopenta[5,6]pyrido[2,3-d]pyrimidin-2-yl)amino)pyrrolidine-1-carboxylate (200 mg, 0.502 mmol) and 4.0 M HCl in 1,4-dioxane (4.00 mL) was stirred at room temperature for 1 h. The resulting mixture was concentrated under reduced pressure. Then it was diluted with MeOH (2.00 mL) and basified to pH 9 with NH₃·H₂O. The residue was purified by Prep-HPLC^{AV} to afford the title compound (115.9 mg, 77.4%, ee>99%) as yellow solid. ¹H NMR (400 MHz, DMSO-d₆) δ 6.93-6.64 (m, 2H), 4.46-4.30 (m, 1H), 3.65-3.50 (m, 1H), 3.15-3.02 (m, 2H), 2.94 (d, J=4.1 Hz, 3H), 2.92-2.76 (m, 5H), 2.59 (s, 3H), 2.11-1.96 (m, 3H), 1.84-1.69 (m, 1H). LCMS^{W} m/z=299 [M+H]⁺.

### Example 232: (3S,4S)-1-methyl-4-((5-methyl-4-(methylamino)-7,8-dihydro-6H-cyclopenta[5,6]pyrido[2,3-d]pyrimidin-2-yl)amino)pyrrolidin-3-ol

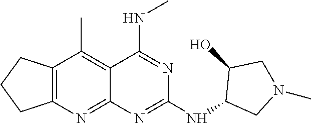 [SMILES: CNc1nc(N[C@H]2CN(C)C[C@@H]2O)nc2nc3c(c(C)c12)CCC3]

### Preparation 121—tert-butyl (3S,4S)-3-hydroxy-4-((5-methyl-4-(methylamino)-7,8-dihydro-6H-cyclopenta[5,6]pyrido[2,3-d]pyrimidin-2-yl)amino)pyrrolidine-1-carboxylate

To a stirred solution of 2-chloro-N,5-dimethyl-7,8-dihydro-6H-cyclopenta[5,6]pyrido[2,3-d]pyrimidin-4-amine (1.50 g, 6.03 mmol) in dioxane (10.0 mL) at room temperature was added tert-butyl (3S,4S)-3-amino-4-hydroxypyrrolidine-1-carboxylate (1.95 g, 9.65 mmol). The resulting mixture was stirred at 100° C. overnight. The resulting mixture was concentrated under reduced pressure. The residue was purified by Prep-HPLC^{AW} to afford the title compound as a yellow solid (950 mg, 38.0%). LCMS m/z=415 [M+H]⁺.

### Preparation 122—(3S,4S)-4-((5-methyl-4-(methylamino)-7,8-dihydro-6H-cyclopenta[5,6]pyrido[2,3-d]pyrimidin-2-yl)amino)pyrrolidin-3-ol

To a stirred solution of tert-butyl (3S,4S)-3-hydroxy-4-((5-methyl-4-(methylamino)-7,8-dihydro-6H-cyclopenta[5,6]pyrido[2,3-d]pyrimidin-2-yl)amino)pyrrolidine-1-carboxylate (450 mg, 1.09 mmol) in dioxane (3.0 mL) at room temperature was added HCl in 1,4-dioxane (7.0 mL, 4.0 M). The resulting mixture was stirred at room temperature for 3 h. The resulting mixture was concentrated under reduced pressure to afford the title compound as a light yellow solid (300 mg, crude).

### Preparation 123—(3S,4S)-1-methyl-4-((5-methyl-4-(methylamino)-7,8-dihydro-6H-cyclopenta[5,6]pyrido[2,3-d]pyrimidin-2-yl)amino)pyrrolidin-3-ol

To a stirred solution of (3S,4S)-4-((5-methyl-4-(methylamino)-7,8-dihydro-6H-cyclopenta[5,6]pyrido[2,3-d]pyrimidin-2-yl)amino)pyrrolidin-3-ol (300 mg, 0.954 mmol) and Et₃N (265 μL, 1.91 mmol) in DCM (6.0 mL) at room temperature was added paraformaldehyde (516 mg, 5.72 mmol). The resulting mixture was stirred at room temperature for 40 min. To the above mixture at room temperature was added STAB (1.21 g, 5.72 mmol). The resulting mixture was stirred at room temperature overnight. The reaction was quenched by the addition of water (15 mL) at room temperature. The resulting mixture was filtered, the filter cake was washed with water (1×10 mL). The filtrate was concentrated under reduced pressure. The mixture was basified to pH 9 with Et₃N. The residue was purified by Prep-HPLC^{M} to afford the title compound as a yellow solid (145 mg, 46.3%). ¹H NMR (400 MHz, Methanol-d₄) δ 4.33-4.12 (m, 2H), 3.18-3.00 (m, 4H), 3.03-2.88 (m, 5H), 2.64 (s, 3H), 2.60-2.49 (m, 2H), 2.37 (s, 3H), 2.13 (p, J=7.6 Hz, 2H). LCMS^{R} m/z=329 [M+H].

### Example 233: N²-((3S,4R)-4-fluoro-1-methylpyrrolidin-3-yl)-N⁴,5-dimethyl-7,8-dihydro-6H-cyclopenta[5,6]pyrido[2,3-d]pyrimidine-2,4-diamine

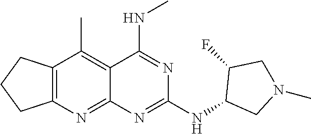 [SMILES: CNc1nc(N[C@H]2CN(C)C[C@H]2F)nc2nc3c(c(C)c12)CCC3]

### Preparation 124—tert-butyl (3R,4S)-3-fluoro-4-((5-methyl-4-(methylamino)-7,8-dihydro-6H-cyclopenta[5,6]pyrido[2,3-d]pyrimidin-2-yl)amino)pyrrolidine-1-carboxylate

Into a 100 mL round-bottom flask were added 2-chloro-N,5-dimethyl-7,8-dihydro-6H-cyclopenta[5,6]pyrido[2,3-d]pyrimidin-4-amine (1.00 g, 4.03 mmol) and tert-butyl (3S,4R)-3-amino-4-fluoropyrrolidine-1-carboxylate (987 mg, 4.84 mmol) in dioxane (20.0 mL) at room temperature. The resulting mixture was stirred at 100° C. for 5 h. The mixture was allowed to cool down to room temperature and concentrated under reduced pressure. The residue was purified by silica gel column chromatography, eluted with DCM/MeOH (12:1) to afford title compound (600 mg, 35.8%) as brown solid. LCMS m/z=417 [M+H]⁺.

### Preparation 125—N²-((3S,4R)-4-fluoro-1-methylpyrrolidin-3-yl)-N⁴,5-dimethyl-7,8-dihydro-6H-cyclopenta[5,6]pyrido[2,3-d]pyrimidine-2,4-diamine

A mixture of tert-butyl (3R,4S)-3-fluoro-4-((5-methyl-4-(methylamino)-7,8-dihydro-6H-cyclopenta[5,6]pyrido[2,3-d]pyrimidin-2-yl)amino)pyrrolidine-1-carboxylate (270 mg, 0.648 mmol) and 37% wt formaldehyde solution (0.270 mL) in formic acid (2.70 mL) was stirred at 100° C. for 1 h. The mixture was allowed to cool down to room temperature and concentrated under reduced pressure. The resulting mixture was diluted with MeOH (4.00 mL) and basified to pH 9 with TEA. The residue was purified by Prep-HPLC^{A} to afford the title compound (87.4 mg, 40.9%, ee>98%) as light orange solid. ¹H NMR (400 MHz, DMSO-d₆) δ 6.99-6.33 (m, 2H), 5.15 (d, J=55.9 Hz, 1H), 4.61-4.44 (m, 1H), 3.24-3.10 (m, 1H), 3.02-2.82 (m, 8H), 2.69-2.63 (m, 1H), 2.60 (s, 3H), 2.57-2.54 (m, 1H), 2.31 (s, 3H), 2.03 (p, J=7.6 Hz, 2H). ¹⁹F NMR (376 MHz, DMSO-d₆) δ −187.85. LCMS^{U} m/z=331 [M+H]⁺.

### Example 234: (R)—N⁴-ethyl-5-methyl-N²-(1-methylpyrrolidin-3-yl)-7,8-dihydro-6H-cyclopenta[5,6]pyrido[2,3-d]pyrimidine-2,4-diamine

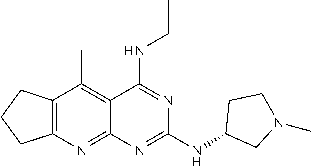 [SMILES: CCNc1nc(N[C@@H]2CCN(C)C2)nc2nc3c(c(C)c12)CCC3]

### Preparation 126—2-chloro-N-ethyl-5-methyl-7,8-dihydro-6H-cyclopenta[5,6]pyrido[2,3-d]pyrimidin-4-amine

To a stirred mixture of 2,4-dichloro-5-methyl-7,8-dihydro-6H-cyclopenta[5,6]pyrido[2,3-d]pyrimidine (1.00 g, 3.94 mmol) in THF (20 mL) was added ethylamine (950 mL, 9.84 mmol, 70% wt. in H₂O). The resulting mixture was stirred at room temperature for 1 h. The precipitated solids were collected by filtration and washed with water (3×10 mL). This resulted in the title compound (600 mg, crude) as brown yellow solid which was used in the next step directly without further purification. LCMS m/z=263 [M+H]⁺.

### Preparation 127—(R)—N⁴-ethyl-5-methyl-N²-(1-methylpyrrolidin-3-yl)-7,8-dihydro-6H-cyclopenta[5,6]pyrido[2,3-d]pyrimidine-2,4-diamine

A mixture of 2-chloro-N-ethyl-5-methyl-7,8-dihydro-6H-cyclopenta[5,6]pyrido[2,3-d]pyrimidin-4-amine (300 mg, 1.14 mmol) and (R)-1-methylpyrrolidin-3-amine (229 mg, 2.28 mmol) in 1,4-dioxane (3.00 mL) was stirred at 100° C. overnight. The mixture was allowed to cool down to room temperature and concentrated under reduced pressure. The residue was purified by Prep-HPLC^{A}N to afford the title compound (18.2 mg, 4.88%, ee>99%) as pink solid. ¹H NMR (400 MHz, Methanol-d₄) δ 4.68-4.56 (m, 1H), 3.61 (q, J=7.0 Hz, 2H), 3.06-2.90 (m, 5H), 2.87-2.71 (m, 1H), 2.70-2.52 (m, 5H), 2.46-2.33 (m, 4H), 2.13 (p, J=7.7 Hz, 2H), 1.86-1.73 (m, 1H), 1.29 (t, J=7.1 Hz, 3H). LCMS^{O} m/z=327 [M+H]⁺.

### Example 235: methyl 4-amino-2-((1-methylpiperidin-4-yl)amino)pyrido[2,3-d]pyrimidine-7-carboxylate

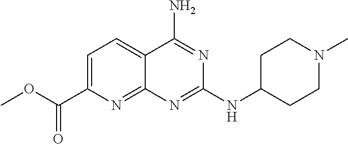

To a stirred solution of 7-chloro-N²-(1-methylpiperidin-4-yl)pyrido[2,3-d]pyrimidine-2,4-diamine (1.00 g, 3.42 mmol) in MeOH (100 mL) and DMF (2.00 mL) were added Pd(dppf)Cl₂ (250 mg, 0.342 mmol) and Et₃N (691 mg, 6.83 mmol) at room temperature. The resulting mixture was stirred under carbon monoxide atmosphere at 100° C. overnight. The resulting mixture was allowed to cool down to room temperature and filtered, the filter cake was washed with MeOH (3×5 mL). The filtrate was concentrated under reduced pressure. The residue was purified by trituration with EtOAc (30 mL). This resulted in the title compound (1.64 g, crude) as an orange solid. ¹H NMR (400 MHz, DMSO-d₆) δ 8.52 (d, J=8.2 Hz, 1H), 7.80-7.30 (m, 3H), 6.88 (d, J=7.2 Hz, 1H), 3.98-3.80 (m, 4H), 2.97-2.85 (m, 2H), 2.34-2.04 (m, 5H), 1.98-1.78 (m, 2H), 1.66-1.49 (m, 2H). LCMS m/z=317 [M+H]⁺

### Example 236: 4-amino-2-((1-methylpiperidin-4-yl)amino)pyrido[2,3-d]pyrimidine-7-carboxylic acid

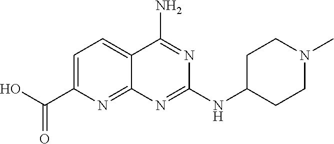 [SMILES: CN1CCC(Nc2nc(N)c3ccc(C(=O)O)nc3n2)CC1]

To a stirred solution of methyl 4-amino-2-((1-methylpiperidin-4-yl)amino)pyrido[2,3-d]pyrimidine-7-carboxylate (50.0 mg, 0.158 mmol) in THF (500 μL) and H₂O (500 μL) were added LiOH—H₂O (66.3 mg, 1.58 mmol) at room temperature. The resulting mixture was stirred at room temperature for 1 h, then it was acidified to pH 5 with 1.0 M HCl (aq.). The resulting mixture was filtered, the filter cake was washed with water (3×2 mL). The filtrate was concentrated under reduced pressure. The residue was purified by Prep-HPLC^{AV} to afford the title compound (25.1 mg, 79.6% over two steps) as a white solid. ¹H NMR (400 MHz, DMSO-d₆) δ 8.41 (d, J=8.1 Hz, 1H), 7.74-7.27 (m, 3H), 6.63 (d, J=8.3 Hz, 1H), 4.01-3.78 (m, 1H), 2.81-2.66 (m, 2H), 2.16 (s, 3H), 1.92 (t, J=11.7, 2H), 1.85-1.70 (m, 2H), 1.65-1.45 (m, 2H). LCMS^{X} m/z=303 [M+H]⁺

### Example 237: 7-(methoxymethyl)-N²-(1-methylpiperidin-4-yl)pyrido[2,3-d]pyrimidine-2,4-diamine

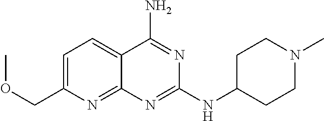 [SMILES: COCc1ccc2c(N)nc(NC3CCN(C)CC3)nc2n1]

To a stirred solution of Example 23 (100 mg, 0.342 mmol) and tributyl(methoxymethyl)stannane (229 mg, 0.684 mmol) in NMP (3.00 mL) was added Pd(PPh₃)₄ (39.5 mg, 0.030 mmol) at room temperature. The resulting mixture was stirred under nitrogen atmosphere at 130° C. for 2 h. The mixture was allowed to cool down to room temperature and purified by Prep-HPLC^{AV} to afford the title compound as a light yellow solid (3.0 mg, 2.90%). ¹H NMR (400 MHz, DMSO-d₆) δ 8.35 (d, J=8.2 Hz, 1H), 7.71-7.17 (br, 2H), 7.03 (d, J=8.1 Hz, 1H), 6.70-6.49 (br, 1H), 4.46 (s, 2H), 3.89-3.71 (m, 1H), 3.38 (s, 3H), 2.80-2.68 (m, 2H), 2.16 (s, 3H), 2.01-1.89 (m, 2H), 1.85-1.76 (m, 2H), 1.58-1.42 (m, 2H). LCMS^{Y} m/z=303 [M+H]⁺.

### Example 238: N²-(1-methylpiperidin-4-yl)-7-(prop-1-en-2-yl) pyrido[2,3-d]pyrimidine-2,4-diamine

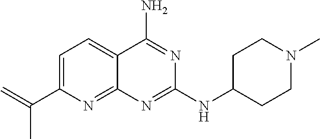 [SMILES: C=C(C)c1ccc2c(N)nc(NC3CCN(C)CC3)nc2n1]

To a stirred solution of Example 23 (300 mg, 1.03 mmol) in dioxane (5.00 mL) and H₂O (1.00 mL) was added prop-1-en-2-ylboronic acid (123 mg, 1.54 mmol), Pd(dppf)Cl₂ (75.0 mg, 0.100 mmol) and K₃PO₄ (435 mg, 2.04 mmol). The resulting mixture was stirred under nitrogen atmosphere at 100° C. for 1 h. The mixture was allowed to cool down to room temperature and concentrated under reduced pressure. The residue was purified by Prep-HPLC^{AV} to afford the title compound as a white solid (190 mg, 62.0%). ¹H NMR (400 MHz, DMSO-d₆) δ 8.31 (d, J=8.1 Hz, 1H), 7.79-7.08 (m, 3H), 6.88-6.37 (m, 1H), 5.96 (s, 1H), 5.42 (s, 1H), 3.98-3.70 (m, 1H), 2.76 (d, J=11.1 Hz, 2H), 2.18 (s, 3H), 2.16 (s, 3H), 2.08-1.88 (m, 2H), 1.88-1.71 (m, 2H), 1.62-1.42 (m, 2H). LCMS m/z=299 [M+H]⁺.

### Example 239: 7-isopropyl-N²-(1-methylpiperidin-4-yl)pyrido[2,3-d]pyrimidine-2,4-diamine

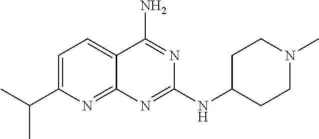 [SMILES: CC(C)c1ccc2c(N)nc(NC3CCN(C)CC3)nc2n1]

A mixture of Example 238 (50.0 mg, 0.168 mmol) and Pd/C (25.0 mg, 10% wt) in MeOH (3.00 mL) was stirred at room temperature under hydrogen atmosphere for 1 h. The mixture was filtered through a short pad of Celite, the filter cake was washed with MeOH (3×5 mL). The filtrate was concentrated under reduced pressure. The residue was purified by Prep-HPLC^{AU} to afford the title compound as a white solid (11.1 mg, 22.0%). ¹H NMR (400 MHz, DMSO-d₆) δ 8.25 (d, J=8.2 Hz, 1H), 7.28 (s, 2H), 6.92 (d, J=8.2 Hz, 1H), 6.47 (s, 1H), 3.91-3.73 (m, 1H), 3.07-2.90 (m, 1H), 2.74 (d, J=11.1 Hz, 2H), 2.16 (s, 3H), 1.96 (t, J=11.5 Hz, 2H), 1.86-1.73 (m, 2H), 1.57-1.43 (m, 2H), 1.23 (d, J=6.9 Hz, 6H). LCMS^{Z} m/z=301 [M+H]⁺.

### Example 240: 2-(4-amino-2-((1-methylpiperidin-4-yl)amino)pyrido[2,3-d]pyrimidin-7-yl)propan-1-ol

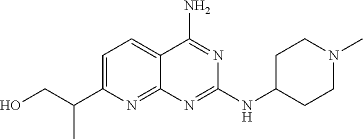 [SMILES: CC(CO)c1ccc2c(N)nc(NC3CCN(C)CC3)nc2n1]

### Preparation 128—2-(4-amino-2-((1-methylpiperidin-4-yl)amino)pyrido[2,3-d]pyrimidin-7-yl)propan-1-ol

To a stirred solution of Example 238 (90.0 mg, 0.302 mmol) in THF (0.50 mL) was added 9-borabicyclo[3.3.1]nonane (2.11 mL, 1.05 mmol, 0.5 M in THF) at 0° C. under nitrogen atmosphere. The resulting mixture was stirred at room temperature under nitrogen atmosphere for 1 h. To the above mixture was added second batches of 9-borabicyclo[3.3.1]nonane (1.2 mL, 0.60 mmol) at 0° C. The mixture was stirred for 1 hours at room temperature and then followed by addition of third batches of 9-borabicyclo[3.3.1]nonane (1.2 mL, 0.60 mmol) 0° C. The resulting mixture was stirred at room temperature overnight. The reaction mixture was cooled to 0° C. and added 30% H₂O₂ (1.03 g, 9.06 mmol) and 1M aq. NaOH (0.45 mL) at 0° C. The resulting mixture was stirred for additional 2 h at room temperature. The reaction was quenched by the addition of Na₂SO₃ (10 mL) at 0° C., then it was concentrated under reduced pressure. The residue was purified by Prep-HPLC^{AV} to afford the title compound (7.60 mg, 8.0%). ¹H NMR (400 MHz, DMSO-d₆) δ 8.24 (d, J=8.2 Hz, 1H), 7.33 (br, 2H), 6.91 (d, J=8.2 Hz, 1H), 6.48 (s, 1H), 4.85-4.56 (m, 1H), 3.93-3.75 (m, 1H), 3.73-3.63 (m, 1H), 3.60-3.50 (m, 1H), 3.00-2.87 (m, 1H), 2.79 (d, J=11.2 Hz, 2H), 2.20 (s, 3H), 2.11-1.97 (m, 2H), 1.87-1.77 (m, 2H), 1.62-1.45 (m, 2H), 1.20 (d, J=6.9 Hz, 3H). LCMS^{W} m/z=317 [M+H]⁺.

### Example 241: 6-methoxy-N²-(1-methylpiperidin-4-yl)pyrido[2,3-d]pyrimidine-2,4-diamine

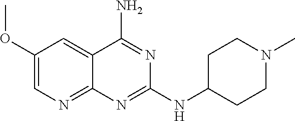 [SMILES: COc1cnc2nc(NC3CCN(C)CC3)nc(N)c2c1]

To a stirred mixture of Example 20 (200 mg, 0.593 mmol) and Pd(PPh₃)₄ (68.5 mg, 0.060 mmol) in methanol (2.00 mL) and dioxane (2.00 mL) were added di-tert-butyl((2-[2,4,6-tris(propan-2-yl)phenyl]phenyl)phosphane (25.2 mg, 0.060 mmol) and Cs₂CO₃ (387 mg, 1.19 mmol) at room temperature. The resulting mixture was stirred under nitrogen atmosphere at 100° C. for 6 h. The mixture was allowed to cool down to room temperature. The resulting mixture was concentrated under reduced pressure. The residue was purified by Prep-HPLC^{N} to afford the title compound as a yellow solid (5.6 mg, 3.25%). ¹H NMR (400 MHz, DMSO-d₆) δ 8.41 (d, J=3.0 Hz, 1H), 7.93 (d, J=3.2 Hz, 1H), 7.34 (s, 2H), 6.35 (s, 1H), 3.81 (s, 3H), 3.78-3.70 (m, 1H), 2.74 (d, J=11.4 Hz, 2H), 2.16 (s, 3H), 2.00-1.89 (m, 2H), 1.86-1.76 (m, 2H), 1.56-1.42 (m, 2H). LCMS^{Z} m/z=289 [M+H]⁺.

### Example 242: 6-(methoxymethyl)-N²-(1-methylpiperidin-4-yl)pyrido[2,3-d]pyrimidine-2,4-diamine

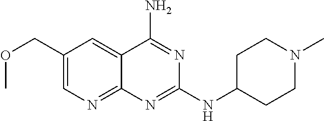 [SMILES: COCc1cnc2nc(NC3CCN(C)CC3)nc(N)c2c1]

### Preparation 129—6-(chloromethyl)-N²-(1-methylpiperidin-4-yl)pyrido[2,3-d]pyrimidine-2,4-diamine

A mixture of Example 123 (50.0 mg, 0.173 mmol) in SOCl₂ (1.00 mL) was stirred at 80° C. for 1 h. The mixture was allowed to cool down to room temperature and concentrated under reduced pressure. This resulted in the title compound (50 mg, crude) as a black solid which was used in the next step directly without further purification. LCMS m/z: 307 [M+H]⁺

### Preparation 130—6-(methoxymethyl)-N₂-(1-methylpiperidin-4-yl)pyrido[2,3-d]pyrimidine-2,4-diamine

A mixture of 6-(chloromethyl)-N²-(1-methylpiperidin-4-yl)pyrido[2,3-d]pyrimidine-2,4-diamine (50.0 mg, 0.163 mmol) and NaOMe in MeOH (30% wt, 1.50 mL) was stirred at 0° C. for 1 h. The resulting mixture was concentrated under reduced pressure. The residue was purified by Prep-HPLC^{L} to afford product (38 mg, crude) as a yellow solid. The crude product was further purified by Prep-HPLC^{AU} to afford the title compound (2.6 mg, 5.3%) as an oyster white solid. ¹H NMR (400 MHz, DMSO-d₆) δ 8.55 (d, J=2.3 Hz, 1H), 8.33 (d, J=2.4 Hz, 1H), 7.40 (br, 2H), 6.58 (br, 1H), 4.40 (s, 2H), 3.85-3.73 (m, 1H), 3.29 (s, 3H), 2.74 (d, J=11.1 Hz, 2H), 2.16 (s, 3H), 1.94 (t, J=11.7 Hz, 2H), 1.81 (d, J=12.3 Hz, 2H), 1.59-1.39 (m, 2H). LCMS^{V} m/z: 303 [M+H]⁺

### Example 243: 6-cyclopentyl-N²-(1-methylpiperidin-4-yl)pyrido[2,3-d]pyrimidine-2,4-diamine

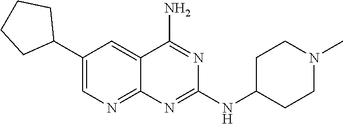 [SMILES: CN1CCC(Nc2nc(N)c3cc(C4CCCC4)cnc3n2)CC1]

A mixture of nickel acetylacetonate (7.62 mg, 0.030 mmol), ([4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate (3.33 mg, 0.003 mmol) and 4-tert-butyl-2-(4-tert-butylpyridin-2-yl)pyridine (7.96 mg, 0.030 mmol), MgCl₂ (28.2 mg, 0.297 mmol) in DMF (4.00 mL) was stirred under nitrogen atmosphere at room temperature for 5 min. Then to the above mixture was added a mixture of Example 20 (100 mg, 0.297 mmol), bromocyclopentane (221 mg, 1.49 mmol) and Et₃N (124 μL, 0.891 mmol) in DMF (4.00 mL). The resulting mixture was under nitrogen atmosphere at room temperature stirred for 5 h under blue LEDs. The solution was purified by Prep-HPLC^{L} to afford the title compound as a light red solid (53.0 mg, crude). The crude product was further purified by Prep-HPLC^{AY} to afford the title compound as an off-white solid (17.6 mg, 18.2%). ¹H NMR (400 MHz, DMSO-d⁶) δ 8.52 (d, J=2.4 Hz, 1H), 8.23 (d, J=2.5 Hz, 1H), 7.38 (s, 2H), 6.44 (s, 1H), 3.84-3.69 (m, 1H), 3.04-2.94 (m, 1H), 2.74 (d, J=11.4 Hz, 2H), 2.16 (s, 3H), 2.09-1.99 (m, 2H), 1.98-1.89 (m, 2H), 1.86-1.74 (m, 4H), 1.72-1.61 (m, 2H), 1.60-1.43 (m, 4H). LCMS^{AA} m/z=327 [M+H]⁺.

### Example 244: 4-amino-2-((1-methylpiperidin-4-yl)amino)pyrido[2,3-d]pyrimidin-5-ol

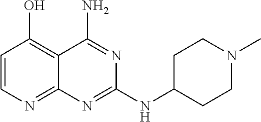 [SMILES: CN1CCC(Nc2nc(N)c3c(O)ccnc3n2)CC1]

### Preparation 131—N-(3-cyano-4-methoxypyridin-2-yl)cyanamide

To a stirred mixture of 2-chloro-4-methoxypyridine-3-carbonitrile (3.75 g, 22.2 mmol) and K₂CO₃ (6.15 g, 44.5 mmol) in dioxane (40.0 mL) at room temperature were added BrettPhos (2.39 g, 4.45 mmol), cyanamide (1.88 g, 44.5 mmol) and BrettPhos Pd G₃ (4.03 g, 4.45 mmol). The resulting mixture was stirred under nitrogen atmosphere at 60° C. for 1 h. The resulting mixture was filtered, the filter cake was washed with MeOH (5×100 mL). The filtrate was concentrated under reduced pressure. The resulting mixture was diluted with water (200 mL) and was acidified to pH 5 with 1 M HCl. The precipitated solids were collected by filtration and washed with water (3×100 mL) to afford the title compound as a white solid (3.20 g, 82.6%). LCMS m/z=175 [M+H]⁺.

### Preparation 132—5-methoxy-N²-(1-methylpiperidin-4-yl)pyrido[2,3-d]pyrimidine-2,4-diamine

A mixture of 2-(cyanoamino)-4-methoxypyridine-3-carbonitrile (3.20 g, 18.4 mmol) and 1-methylpiperidin-4-amine (4.20 g, 36.8 mmol) in dioxane (60.0 mL) was stirred at 120° C. overnight. The resulting mixture was concentrated under reduced pressure. The residue was purified by trituration with EtOAc (200 mL) to afford the title compound as a yellow solid (2.48 g, 46.8%). LCMS m/z=289 [M+H]⁺.

### Preparation 133—4-amino-2-((1-methylpiperidin-4-yl)amino)pyrido[2,3-d]pyrimidin-5-ol

Into a 100 mL round-bottom flask were added 5-methoxy-N²-(1-methylpiperidin-4-yl)pyrido[2,3-d]pyrimidine-2,4-diamine (2.38 g, 8.25 mmol) and aq. HCl (25.0 mL, 6.0 M) at room temperature. The resulting mixture was stirred at 100° C. for 2 h. The resulting mixture was concentrated under reduced pressure to afford the title compound as a yellow solid (3.00 g, crude). ¹H NMR (400 MHz, DMSO-d₆) δ 11.41-10.83 (m, 1H), 9.52-9.13 (m, 1H), 7.59-7.00 (m, 2H), 6.78 (d, J=8.2 Hz, 1H), 5.80 (d, J=7.6 Hz, 1H), 3.81-3.63 (m, 1H), 2.73 (d, J=11.1 Hz, 2H), 2.12 (s, 3H), 1.88 (t, J=13.2, 2H), 1.76 (d, J=12.2 Hz, 2H), 1.56-1.28 (m, 2H). LCMS m/z=275 [M+H]⁺.

### Example 245: N²-(1-methylpiperidin-4-yl)-5-(prop-1-en-2-yl)pyrido[2,3-d]pyrimidine-2,4-diamine

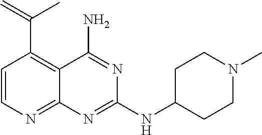 [SMILES: C=C(C)c1ccnc2nc(NC3CCN(C)CC3)nc(N)c12]

### Preparation 134—5-chloro-N²-(1-methylpiperidin-4-yl)pyrido[2,3-d]pyrimidine-2,4-diamine

Into a 100 mL round-bottom flask were added 4-amino-2-[(1-methylpiperidin-4-yl)amino]pyrido[2,3-d]pyrimidin-5-ol (2.50 g, 9.11 mmol) and POCl₃ (30.0 mL) at room temperature. The resulting mixture was stirred at 80° C. for 1 h. The resulting mixture was concentrated under reduced pressure. The reaction was quenched with Water/Ice at 0° C. The mixture was basified to pH 8 with saturated NaHCO₃ (aq.). The aqueous layer was extracted with CH₂Cl₂/MeOH (10:1, 3×100 mL) to afford the title compound as a yellow solid (1.05 g, 39.4%). LCMS m/z=293 [M+H]⁺.

### Preparation 135—N²-(1-methylpiperidin-4-yl)-5-(prop-1-en-2-yl)pyrido[2,3-d]pyrimidine-2,4-diamine

To a stirred mixture of 5-chloro-N²-(1-methylpiperidin-4-yl)pyrido[2,3-d]pyrimidine-2,4-diamine (150 mg, 0.512 mmol) and K₃PO₄ (285 mg, 1.34 mmol) in dioxane (5.00 mL) and H₂O (1.00 mL) were added Pd(dppf)Cl₂ (45.0 mg, 0.0610 mmol) and 4,4,5,5-tetramethyl-2-(prop-1-en-2-yl)-1,3,2-dioxaborolane (225 uL, 1.21 mmol) at room temperature. The resulting mixture was stirred at 100° C. under nitrogen atmosphere for 1 h. The resulting mixture was filtered, the filter cake was washed with MeOH (2×5 mL). The filtrate was concentrated under reduced pressure. The residue was purified by Prep-HPLC^{Av} to afford the title compound as a white solid (100 mg, 65.4%). ¹H NMR (400 MHz, DMSO-d₆) δ 8.52 (d, J=4.5 Hz, 1H), 7.34-6.20 (m, 4H), 5.43 (s, 1H), 5.15 (s, 1H), 3.87-3.70 (m, 1H), 2.79-2.64 (m, 2H), 2.16 (s, 3H), 2.09 (s, 3H), 1.94 (t, J=11.5 Hz, 2H), 1.87-1.69 (m, 2H), 1.64-1.42 (m, 2H). LCMS m/z=299 [M+H]⁺.

### Example 246: 5-isopropyl-N²-(1-methylpiperidin-4-yl)pyrido[2,3-d]pyrimidine-2,4-diamine

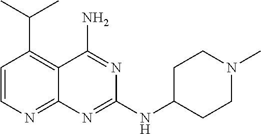 [SMILES: CC(C)c1ccnc2nc(NC3CCN(C)CC3)nc(N)c12]

A mixture of Example 245 (40.0 mg, 0.135 mmol) and 10% Pd/C (40 mg) in EA (16.0 mL) was stirred at room temperature under hydrogen atmosphere for 8 h. The resulting mixture was filtered, the filter cake was washed with MeOH (3×10 mL). The filtrate was concentrated under reduced pressure. The residue was purified silica gel column chromatography, eluted with CH₂Cl₂/MeOH/Et₃N (10:1:0.06) to afford the title compound as a white solid (17.5 mg, 43.4%). ¹H NMR (400 MHz, DMSO-d₆) δ 8.53 (d, J=4.9 Hz, 1H), 7.91-7.25 (m, 1H), 7.14-6.52 (m, 3H), 4.12-3.93 (m, 1H), 3.85-3.69 (m, 1H), 3.08-2.81 (m, 2H), 2.67 (s, 3H), 2.13-1.97 (m, 2H), 1.92-1.69 (m, 2H), 1.34-1.08 (m, 7H), 0.98-0.73 (m, 1H). LCMS^{E} m/z=301 [M+H]⁺.

### Example 247: N-methyl-N2-(1-methylpiperidin-4-yl)pyrido[2,3-d]pyrimidine-2,4,5-triamine

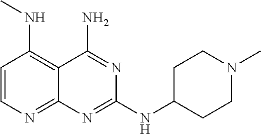 [SMILES: CNc1ccnc2nc(NC3CCN(C)CC3)nc(N)c12]

### Preparation 136—N⁵-(4-methoxybenzyl)-N⁵-methyl-N²-(1-methylpiperidin-4-yl)pyrido[2,3-d]pyrimidine-2,4,5-triamine

A mixture of 5-chloro-N²-(1-methylpiperidin-4-yl)pyrido[2,3-d]pyrimidine-2,4-diamine (150 mg, 0.512 mmol) and 1-(4-methoxyphenyl)-N-methylmethanamine (2.00 mL) was stirred at 100° C. for 1 h. The mixture was allowed to cool down to room temperature and was purified by Prep-HPLC^{AV} to afford the title compound (150 mg, 71.8%) as a yellow solid. LCMS m/z=408 [M+H]⁺.

### Preparation 137—N⁵-methyl-N²-(1-methylpiperidin-4-yl)pyrido[2,3-d]pyrimidine-2,4,5-triamine

A solution of N⁵-(4-methoxybenzyl)-N⁵-methyl-N²-(1-methylpiperidin-4-yl)pyrido[2,3-d]pyrimidine-2,4,5-triamine (150 mg, 0.368 mmol) in TFA (2.00 mL) was stirred at 100° C. for 1 h. The mixture was allowed to cool down to room temperature and concentrated under reduced pressure. The residue was basified to pH 9 with NH₃—H₂O. The residue was purified by Prep-HPLC^{AV} to afford the title compound (44.0 mg, 41.6%) as a white solid. ¹H NMR (400 MHz, DMSO-d₆) δ 11.07-9.69 (m, 2H), 7.47-6.45 (m, 2H), 6.27 (s, 1H), 5.95 (s, 1H), 3.76-3.59 (m, 1H), 2.89 (s, 3H), 2.72 (d, J=11.1 Hz, 2H), 2.14 (s, 3H), 1.90 (t, J=11.6 Hz, 2H), 1.77 (d, J=11.8 Hz, 2H), 1.55-1.39 (m, 2H). LCMS^{AB} m/z=288 [M+H]⁺.

### Example 248: 5-(methoxymethyl)-N²-(1-methylpiperidin-4-yl)pyrido[2,3-d]pyrimidine-2,4-diamine

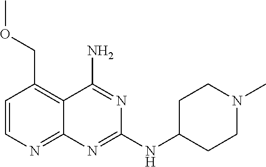 [SMILES: COCc1ccnc2nc(NC3CCN(C)CC3)nc(N)c12]

To a stirred mixture of 5-chloro-N²-(1-methylpiperidin-4-yl)pyrido[2,3-d]pyrimidine-2,4-diamine (70.0 mg, 0.239 mmol) and tributyl(methoxymethyl)stannane (160 mg, 0.478 mmol) in NMP (8.00 mL) was added Pd(PPh₃)₂Cl₂ (16.7 mg, 0.0240 mmol) and 4 Å MS (140 mg). The resulting mixture was stirred under nitrogen atmosphere at 120° C. for 4 h. The resulting mixture was allowed to cool down to room temperature, then was diluted with saturated KF (aq.) (20 mL). The resulting mixture was extracted with CH₂Cl₂ (3×100 mL). The combined organic layers were dried over anhydrous Na₂SO₄. After filtration, the filtrate was concentrated under reduced pressure. The residue was purified by Prep-H PLC^{AV} to afford the title compound as a brown solid (6.5 mg, 8.99%). ¹H NMR (400 MHz, Methanol-d₄) δ 8.64-8.51 (m, 1H), 7.03 (d, J=4.7 Hz, 1H), 4.71 (s, 2H), 4.04-3.93 (m, 1H), 3.45 (s, 3H), 2.88 (d, J=11.8 Hz, 2H), 2.31 (s, 3H), 2.28-2.19 (m, 2H), 2.09-2.01 (m, 2H), 1.68-1.54 (m, 2H). LCMS^{AC} m/z=303 [M+H]⁺.

### Example 249: 5-ethynyl-N²-(1-methylpiperidin-4-yl)pyrido[2,3-d]pyrimidine-2,4-diamine

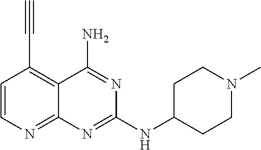 [SMILES: C#Cc1ccnc2nc(NC3CCN(C)CC3)nc(N)c12]

To a stirred mixture of 5-chloro-N²-(1-methylpiperidin-4-yl)pyrido[2,3-d]pyrimidine-2,4-diamine (200 mg, 0.683 mmol) and CuI (13.0 mg, 0.0680 mmol) in DMF (4.0 mL) were added Pd(dppf)Cl₂ (50.0 mg, 0.0680 mmol) and Et₃N (346 mg, 3.415 mmol). To the above mixture was added trimethylsilylacetylene (335 mg, 3.42 mmol) at room temperature. The resulting mixture was stirred under nitrogen atmosphere at 80° C. for 3 h. The mixture was allowed to cool down to room temperature and was filtered, the filter cake was washed with MeOH (3×5 mL). The filtrate was concentrated under reduced pressure. The residue was purified by Prep-HPLC^{AV} to afford the title compound as a white solid as an off-white solid (3.2 mg, 1.62%). ¹H NMR (400 MHz, DMSO-d₆) δ 8.21-7.56 (m, 2H), 6.89 (d, J=7.8 Hz, 1H), 6.55 (s, 2H), 3.93-3.73 (m, 1H), 3.05-2.81 (m, 2H), 2.41-2.19 (m, 4H), 1.91 (d, J=12.8 Hz, 2H), 1.67-1.53 (m, 2H), 1.27-1.16 (m, 2H). LCMS^{AD} m/z=283 [M+H]⁺.

### Example 250: 4-amino-2-((1-methylpiperidin-4-yl)amino)pyrido[2,3-d]pyrimidine-5-carbonitrile

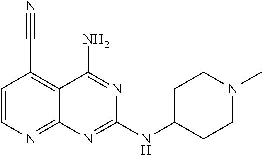 [SMILES: CN1CCC(Nc2nc(N)c3c(C#N)ccnc3n2)CC1]

To a stirred solution of 5-chloro-N²-(1-methylpiperidin-4-yl)pyrido[2,3-d]pyrimidine-2,4-diamine (80.0 mg, 0.273 mmol) in NMP (2.00 mL) were added Zn(CN)₂ (64.1 mg, 0.546 mmol), Zn (35.7 mg, 0.546 mmol), Pd₂(dba)₃ (25.0 mg, 0.0270 mmol) and dppf (15.0 mg, 0.0270 mmol). The resulting mixture was stirred under nitrogen atmosphere at 100° C. for 1 h. The resulting mixture was filtered, the filter cake was washed with MeOH (3×10 mL). The filtrate was concentrated under reduced pressure. The residue was purified by Prep-HPLC^{AX} to afford the title compound as a light yellow solid (21.0 mg, 27.1%). ¹H NMR (400 MHz, DMSO-d₆) δ 8.78 (d, J=4.5 Hz, 1H), 7.57-7.30 (m, 2H), 7.27-7.02 (m, 2H), 3.86-3.70 (m, 1H), 2.75 (d, J=11.1 Hz, 2H), 2.16 (s, 3H), 1.94 (t, J=11.6 Hz, 2H), 1.80 (d, J=12.2 Hz, 2H), 1.61-1.46 (m, 2H). LCMS^{AE} m/z=284 [M+H]⁺.

### Example 251: N²-(1-methylpiperidin-4-yl)-5-(prop-1-yn-1-yl)pyrido[2,3-d]pyrimidine-2,4-diamine

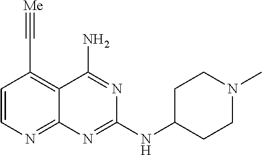

To a stirred mixture of 5-chloro-N²-(1-methylpiperidin-4-yl)pyrido[2,3-d]pyrimidine-2,4-diamine (100 mg, 0.342 mmol) and Pd(dppf)Cl₂ (25.0 mg, 0.034 mmol) in DMF (4.00 mL) was added tributyl(prop-1-yn-1-yl)stannane (562 mg, 1.71 mmol). The mixture was stirred under nitrogen atmosphere at 120° C. for 1 h. The resulting mixture was allowed to cool down to room temperature and concentrated under reduced pressure. The residue was purified by Prep-HPLC^{AV} to afford the title compound (20.1 mg, 19.9%) as a white solid. ¹H NMR (400 MHz, DMSO-d₆) δ 11.41 (br, 1H), 7.52 (br, 1H), 6.65 (s, 1H), 6.40 (s, 2H), 3.86-3.72 (m, 1H), 2.76 (d, J=11.4 Hz, 2H), 2.29 (s, 3H), 2.17 (s, 3H), 2.06-1.88 (m, 2H), 1.83 (d, J=12.3 Hz, 2H), 1.59-1.44 (m, 2H). LCMS^{AF} m/z=297 [M+H]⁺.

### Example 252: 5-(1-methoxyethyl)-N²-(1-methylpiperidin-4-yl)pyrido[2,3-d]pyrimidine-2,4-diamine

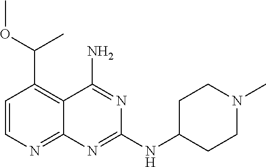 [SMILES: COC(C)c1ccnc2nc(NC3CCN(C)CC3)nc(N)c12]

### Preparation 138—5-(1-methoxyvinyl)-N²-(1-methylpiperidin-4-yl)pyrido[2,3-d]pyrimidine-2,4-diamine

To a stirred mixture of 5-chloro-N²-(1-methylpiperidin-4-yl)pyrido[2,3-d]pyrimidine-2,4-diamine (100 mg, 0.342 mmol) and Pd(PPh₃)₄ (39.5 mg, 0.0340 mmol) in NMP (2.00 mL) was added tributyl(1-methoxyethenyl)stannane (237 μL, 0.684 mmol) at room temperature. The resulting mixture was stirred under nitrogen atmosphere at 120° C. for overnight. The reaction was quenched with sat. KF (aq.) at room temperature. The resulting mixture was extracted with CH₂Cl₂ (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with CH₂Cl₂/MeOH/NH₃·H₂O (10:1:0.1) to afford the title compound as a brown semi-solid (60.0 mg, 55.8%). LCMS m/z=315 [M+H]⁺.

### Preparation 139—5-(1-methoxyethyl)-N²-(1-methylpiperidin-4-yl)pyrido[2,3-d]pyrimidine-2,4-diamine

A solution of 5-(1-methoxyvinyl)-N²-(1-methylpiperidin-4-yl)pyrido[2,3-d]pyrimidine-2,4-diamine (56.0 mg, 0.178 mmol) and 20% Pd(OH)₂/C (56.0 mg) in MeOH (5.00 mL) was stirred at room temperature under hydrogen atmosphere overnight. The resulting mixture was filtered through a short pad of Celite, the filter cake was washed with MeOH (3×10 mL). The filtrate was concentrated under reduced pressure. The residue was purified by Prep-HPLC^{AV} to afford the title compound as an off-white solid (5.6 mg, 9.70%). ¹H NMR (400 MHz, Methanol-d₄) δ 8.58 (d, J=4.7 Hz, 1H), 7.07 (d, J=4.7 Hz, 1H), 4.73 (q, J=6.8 Hz, 1H), 4.16-4.03 (m, 1H), 3.36 (s, 3H), 3.25-3.10 (m, 2H), 2.80-2.63 (m, 2H), 2.59 (s, 3H), 2.23-2.11 (m, 2H), 1.81-1.68 (m, 2H), 1.58 (d, J=6.8 Hz, 3H). LCMS^{I} m/z=317 [M+H]⁺.

### Example 253: 5-methyl-N2-(3-morpholinopropyl)-7-(m-tolyl)pyrido[2,3-d]pyrimidine-2,4-diamine

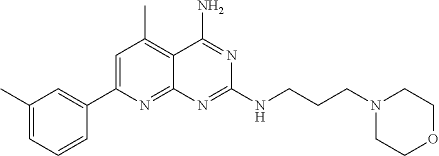 [SMILES: Cc1cccc(-c2cc(C)c3c(N)nc(NCCCN4CCOCC4)nc3n2)c1]

### Preparation 140—methyl 2-amino-4,6-dihydroxypyridine-3-carboxylate

To a stirred solution of 1,5-dimethyl 3-oxopentanedioate (20.0 g, 115 mmol) and aminoformonitrile (5.79 g, 138 mmol) in DME (200 mL) was added nickel acetylacetonate (150 mg, 0.574 mmol) at room temperature. The resulting mixture was stirred at 80° C. overnight. The mixture was allowed to cool down to room temperature. The precipitated solids were collected by filtration and washed with DME (3×2 mL). The resulting solids was diluted with MeOH (100 mL) and was stirred for 30 min at room temperature. The precipitated solids were collected by filtration and washed with MeOH (3×10 mL). The solid was dried under vacuum. This resulted in the title compound (8.80 g, 41.6%) as a tan solid. LCMS m/z=185 [M+H]⁺.

### Preparation 141—methyl 2-amino-4,6-dichloronicotinate

To a stirred mixture of methyl 2-amino-4,6-dihydroxypyridine-3-carboxylate (3.00 g, 16.3 mmol) in POCl₃ (45.0 mL) was added DIEA (9.00 mL) at room temperature. The resulting mixture was stirred at 60° C. overnight. The mixture was allowed to cool down to room temperature and concentrated under vacuum. The residue was added to the NaHCO₃(aq.) dropwise at 0° C. The resulting mixture was extracted with EtOAc (3×100 mL). The combined organic layers were washed with brine, dried over anhydrous Na₂SO₄. After filtration, the filtrate was concentrated under reduced pressure. This resulted in the title compound (2.0 g, crude) as a brown solid. LCMS m/z=221 [M+H]⁺.

### Preparation 142—methyl 2-amino-4-chloro-6-(m-tolyl)nicotinate

To a stirred mixture of methyl 2-amino-4,6-dichloropyridine-3-carboxylate (2.03 g, 9.18 mmo) and 3-methylphenylboronic acid (1.50 g, 11.0 mmol) in 1,4-dioxane (40.0 mL) and H₂O (4.00 mL) at room temperature were added Pd(PPh₃)₄ (1.06 g, 0.918 mmol) and K₃PO₄ (2.92 g, 13.8 mmol). The resulting mixture was stirred under nitrogen atmosphere at 60° C. for 3 h. The mixture was allowed to cool down to room temperature and diluted with H₂O (100 mL). The resulting mixture was extracted with EtOAc (3×50 mL). The combined organic layers were washed with brine, dried over anhydrous Na₂SO₄. After filtration, the filtrate was concentrated under reduced pressure. The residue was purified by silica gel column chromatography, eluted with PE/EA (10:1) to afford the title compound (1.38 g, 54.3%) as a yellow solid. LCMS m/z=277 [M+H]⁺.

### Preparation 143—methyl 2-amino-4-methyl-6-(m-tolyl)nicotinate

To a stirred mixture of methyl 2-amino-4-chloro-6-(m-tolyl)nicotinate (1.24 g, 4.48 mmol) and methylboronic acid (805 mg, 13.4 mmol) in 1,4-dioxane (18.0 mL) at room temperature were added Pd(dppf)Cl₂·CH₂Cl₂ (365 mg, 0.448 mmol) and K₃PO₄ (2.85 g, 13.4 mmol). The resulting mixture was stirred under nitrogen atmosphere at 110° C. for 1 h. The mixture was allowed to cool down to room temperature. The resulting mixture was diluted with H₂O (50 mL) and extracted with EtOAc (3×50 mL). The combined organic layers were washed with brine and dried over anhydrous Na₂SO₄. After filtration, the filtrate was concentrated under reduced pressure. The residue was purified by silica gel column chromatography, eluted with PE/EA (2:1) to afford the title compound (606 mg, 52.7%) as a yellow solid. LCMS m/z=257 [M+H]⁺.

### Preparation 144—5-methyl-7-(m-tolyl)pyrido[2,3-d]pyrimidine-2,4(1H,3H)-dione

A solution of methyl 2-amino-4-methyl-6-(m-tolyl)nicotinate (576 mg, 2.25 mmol) and 2,2,2-trichloroethanecarbonyl isocyanate (423 mg, 2.25 mmol) in THF (9.00 mL) was stirred under nitrogen atmosphere at room temperature for 30 min. The resulting mixture was concentrated under reduced pressure. To the above mixture was added 7.0 M NH₃ in MeOH (9.00 mL) at room temperature. The resulting mixture was stirred for additional 30 min at room temperature. The resulting mixture was concentrated under reduced pressure. The residue was purified by trituration with MTBE (20 mL). This resulted in the title compound (570 mg, 94.8%) as a white solid. LCMS m/z=268 [M+H]⁺.

### Preparation 145—2,4-dichloro-5-methyl-7-(m-tolyl)pyrido[2,3-d]pyrimidine

A mixture of 5-methyl-7-(m-tolyl)pyrido[2,3-d]pyrimidine-2,4(1H,3H)-dione (500 mg, 1.87 mmol) and DIEA (2.00 mL) in POCl₃ (10 mL) was stirred at 100° C. for 1 h. The mixture was allowed to cool down to room temperature. The resulting mixture was concentrated under reduced pressure. The crude product was used in the next step directly without further purification. LCMS m/z=304 [M+H]⁺.

### Preparation 146—2-chloro-5-methyl-7-(m-tolyl)pyrido[2,3-d]pyrimidin-4-amine

To a stirred mixture of 2,4-dichloro-5-methyl-7-(m-tolyl)pyrido[2,3-d]pyrimidine (300 mg, 0.986 mmol) in ACN (12.0 mL) at 0° C. was added NH₃—H₂O (3.00 mL). The resulting mixture was stirred at 0° C. for 1 h. The resulting mixture was concentrated under reduced pressure. The residue was dissolved in MeOH (20 mL). The resulting mixture was filtered, the filter cake was washed with MeOH (3×2 mL). The filtrate was concentrated under reduced pressure. The residue was dissolved in ACN (20 mL). The solid was filtered out, the filter cake was washed with ACN (3×2 mL). The filtrate was concentrated under reduced pressure. This resulted in the title compound (166 mg, crude) as a brown solid. LCMS m/z=285 [M+H]⁺.

### Preparation 147—5-methyl-N²-(3-morpholinopropyl)-7-(m-tolyl)pyrido[2,3-d]pyrimidine-2,4-diamine

To a stirred mixture of 2-chloro-5-methyl-7-(m-tolyl)pyrido[2,3-d]pyrimidin-4-amine (70.0 mg, 0.246 mmol) and 4-morpholinepropanamine (70.9 mg, 0.492 mmol) in 1,4-dioxane (4.00 mL) was added CsF (74.7 mg, 0.492 mmol). The resulting mixture was stirred at 100° C. overnight. The mixture was allowed to cool down to room temperature. The resulting mixture was concentrated under vacuum. The residue was purified by Prep-HPLC^{AY} to afford the title compound as a brown yellow solid (7.2 mg, 7.46%). ¹H NMR (400 MHz, DMSO-d₆) δ 7.99 (s, 1H), 7.93 (d, J=7.8 Hz, 1H), 7.50-7.21 (m, 2H), 7.29 (d, J=7.5 Hz, 1H), 6.80 (s, 2H), 6.62 (s, 1H), 3.59 (t, J=4.6 Hz, 4H), 3.46-3.38 (m, 2H), 2.76 (s, 3H), 2.41 (s, 3H), 2.39-2.32 (m, 6H), 1.77-1.65 (m, 2H). LCMS^{A}E m/z=393 [M+H]⁺.

### Example 254: N²-(4-(dimethylamino)butyl)-5,7-dimethyl-1,8-naphthyridine-2,4-diamine

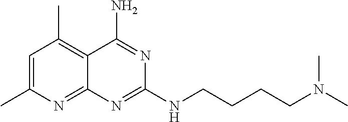

### Preparation 148—ethyl 2-amino-4,6-dimethylnicotinate

To a stirred solution of ethyl 3-amino-3-iminopropanoate hydrochloride (10.0 g, 60.0 mmol) in EtOH (90.0 mL) was added piperidine (15.3 g, 180 mmol) and pentane-2,4-dione (9.01 g, 90.0 mmol) dropwise at room temperature. The resulting mixture was stirred at room temperature for 16 h. The resulting mixture was diluted with water (300 mL) and extracted with EtOAc (3×150 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (10:1) to afford the title compound as a light yellow solid (8.0 g, 68.6%). LCMS m/z=195 [M+H]⁺.

### Preparation 149—5,7-dimethyl-1,8-naphthyridine-2,4-diol

To a stirred solution of ethyl 2-amino-4,6-dimethylnicotinate (6.20 g, 31.9 mmol) in ethyl acetate (200 mL) was added NaH (8.94 g, 223 mmol, 60% in oil) in portions at room temperature. The resulting mixture was stirred at 80° C. for 3 h. The mixture was allowed to cool down to room temperature. The reaction was quenched with Water/Ice at 0° C. and extracted with EtOAc (100 mL×3). The aqueous layer was acidified to pH 6 with AcOH. The precipitated solids were collected by filtration and washed with water (30 mL×3). The solid was dried under vacuum. This resulted in the title compound as an off-white solid (4.8 g, 79.1%). LCMS m/z=191 [M+H]⁺

### Preparation 150—2,4-dichloro-5,7-dimethyl-1,8-naphthyridine

To a stirred mixture of 5,7-dimethyl-1,8-naphthyridine-2,4-diol (4.30 g, 22.6 mmol) in Toluene (130 mL) was added POCl₃ (21.1 mL, 226 mmol) and DIEA (11.8 mL, 67.8 mmol) dropwise at 0° C. The resulting mixture was stirred at 100° C. for 8 h. The mixture was allowed to cool down to room temperature. The resulting mixture was added into saturated NaHCO₃(aq.) at 0° C. The resulting mixture was extracted with EtOAc (3×200 mL). The combined organic layers were washed with brine (2×300 mL), dried over anhydrous Na₂SO₄. After filtration, the filtrate was concentrated under reduced pressure. The residue was purified by silica gel column chromatography, eluted with PE/EA (5:1) to afford the title compound as a red solid (3.7 g, 72.1%). LCMS m/z=227 [M+H]⁺.

### Preparation 151—2-chloro-5,7-dimethyl-1,8-naphthyridin-4-amine

A solution of 2,4-dichloro-5,7-dimethyl-1,8-naphthyridine (600 mg, 2.64 mmol) and CsF (1.20 g, 7.93 mmol) in NH₃—H₂O (20.0 mL) and dioxane (20.0 mL) was stirred at 100° C. for 34 h. The mixture was allowed to cool down to room temperature. The resulting mixture was concentrated under reduced pressure. The residue was purified by Prep-HPLC^{AV} to afford 2-chloro-5,7-dimethyl-1,8-naphthyridin-4-amine as yellow solid (555 mg, crude) and 4-chloro-5,7-dimethyl-1,8-naphthyridin-2-amine as yellow solid (430 mg, crude, isomer). LCMS m/z=208 [M+H]⁺.

### Preparation 152—N²-(4-(dimethylamino)butyl)-5,7-dimethyl-1,8-naphthyridine-2,4-diamine

To a stirred solution of 2-chloro-5,7-dimethyl-1,8-naphthyridin-4-amine (50.0 mg, 0.240 mmol) and (4-aminobutyl)dimethylamine (280 mg, 2.41 mmol) in dioxane (1.00 mL) were added Cs₂CO₃ (157 mg, 0.480 mmol) and Pd-PEPPSI-lHeptCl 3-chloropyridine (23.5 mg, 0.020 mmol) at room temperature. The resulting mixture was stirred under nitrogen atmosphere at 100° C. overnight. The mixture was allowed to cool down to room temperature. The resulting mixture was concentrated under reduced pressure. The residue was purified by Prep-TLC (EA/MeOH=1:2) to afford the title compound (1.8 mg, 2.36%). ¹H NMR (400 MHz, DMSO-d₆) δ 6.62 (s, 1H), 6.54 (s, 1H), 5.82 (s, 1H), 5.73 (s, 2H), 3.28-3.24 (m, 2H), 2.69 (s, 3H), 2.37 (s, 3H), 2.21 (t, J=6.9 Hz, 2H), 2.11 (s, 6H), 1.58-1.41 (m, 4H). LCMS^{AB} m/z=288 [M+H]⁺.

### Example 255: (R)-5,7-dimethyl-N²-(pyrrolidin-3-yl)-1,8-naphthyridine-2,4-diamine

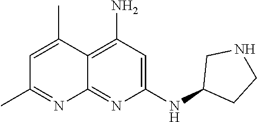 [SMILES: Cc1cc(C)c2c(N)cc(N[C@@H]3CCNC3)nc2n1]

### Preparation 153—tert-butyl (R)-3-((4-amino-5,7-dimethyl-1,8-naphthyridin-2-yl)amino)pyrrolidine-1-carboxylate

To a stirred mixture of 2-chloro-5,7-dimethyl-1,8-naphthyridin-4-amine (500 mg, 2.41 mmol) and tert-butyl (R)-3-aminopyrrolidine-1-carboxylate (897 mg, 4.82 mmol) in dioxane (10.0 mL) were added Cs₂CO₃ (1.57 g, 4.82 mmol) and (SP-4-1)-[1,3-Bis[2,6-bis(1-ethylpropyl)phenyl]-4,5-dichloro-1,3-dihydro-2H-imidazol-2-ylidene]dichloro(2-methylpyridine)palladium (203 mg, 0.241 mmol) at room temperature. The resulting mixture was stirred under nitrogen atmosphere at 100° C. for 1 h. The mixture was allowed to cool down to room temperature and concentrated under reduced pressure. The residue was purified by Prep-HPLC^{AV} to afford the title compound (80.0 mg, 9.29%) as a white solid. LCMS m/z=358 [M+H]⁺.

### Preparation 154—(R)-5,7-dimethyl-N²-(pyrrolidin-3-yl)-1,8-naphthyridine-2,4-diamine

A mixture of tert-butyl (R)-3-((4-amino-5,7-dimethyl-1,8-naphthyridin-2-yl)amino)pyrrolidine-1-carboxylate (75.0 mg, 0.210 mmol) and 4.0 M HCl in 1,4-dioxane (1.00 mL) was stirred at room temperature for 1 h. The resulting mixture was concentrated under reduced pressure and dissolved in MeOH (1.00 mL). The mixture was basified to pH 9 with NH₃·H₂O. The residue was purified by Prep-HPLC^{AV} to afford the title compound (22.9 mg, 42.4%, ee>99%) as an off-white solid. ¹H NMR (400 MHz, DMSO-d₆) δ 8.88 (br, 1H), 6.95 (d, J=5.9 Hz, 1H), 6.71 (s, 1H), 5.97 (s, 2H), 5.85 (s, 1H), 4.54-4.31 (m, 1H), 3.47-3.38 (m, 2H), 3.19-3.03 (m, 2H), 2.71 (s, 3H), 2.39 (s, 3H), 2.26-2.17 (m, 1H), 1.92-1.81 (m, 1H). LCMS^{T} m/z=258 [M+H]⁺.

### Example 256: (R)—N⁴,5,7-trimethyl-N²-(pyrrolidin-3-yl)-1,8-naphthyridine-2,4-diamine

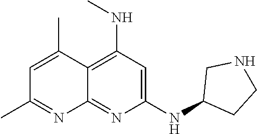 [SMILES: CNc1cc(N[C@@H]2CCNC2)nc2nc(C)cc(C)c12]

### Preparation 155—2-chloro-N,5,7-trimethyl-1,8-naphthyridin-4-amine

To a stirred mixture of 2,4-dichloro-5,7-dimethyl-1,8-naphthyridine (3.00 g, 13.2 mmol) in DMSO (30.0 mL) was added CH₃NH₂·HCl (1.34 g, 20.0 mmol) and DIEA (3.41 g, 26.4 mmol) at room temperature. The resulting mixture was stirred at 100° C. for 4 h. The resulting mixture was purified by Prep-HPLC^{AV} to afford the title compound (1.10 g, 37.6%) as a white solid. LCMS m/z=222 [M+H]⁺

### Preparation 156—tert-butyl (R)-3-((5,7-dimethyl-4-(methylamino)-1,8-naphthyridin-2-yl)amino)pyrrolidine-1-carboxylate

A mixture of 2-chloro-N,5,7-trimethyl-1,8-naphthyridin-4-amine (300 mg, 1.35 mmol) and tert-butyl (R)-3-aminopyrrolidine-1-carboxylate (3.00 mL) was stirred at 140° C. for 5 h. The mixture was allowed to cool down to room temperature. The resulting mixture was diluted with DMF (5 mL). The residue was purified by Prep-HPLC^{AV} to afford the title compound (408 mg, 81.0%) as a green solid. LCMS m/z=372 [M+H]⁺

### Preparation 157—(R)—N⁴,5, 7-trimethyl-N²-(pyrrolidin-3-yl)-1,8-naphthyridine-2,4-diamine

To a stirred mixture of tert-butyl (R)-3-((5,7-dimethyl-4-(methylamino)-1,8-naphthyridin-2-yl)amino)pyrrolidine-1-carboxylate (390 mg, 1.05 mmol) in 1,4-dioxane (4.00 mL) was added HCl(gas) in 1,4-dioxane (4.00 mL, 4.0 M) dropwise at room temperature. The resulting mixture was stirred at room temperature for 1 h and concentrated under reduced pressure. The residue was dissolved in DMSO (4 mL) and basified to pH 9 with NH₃·H₂O. The residue was purified by Prep-HPLC^{AZ} to afford the title compound (41.1 mg, 11.7%, ee>99%) as a white solid TFA salt. ¹H NMR (400 MHz, DMSO-d₆) δ 8.36 (s, 1H), 7.40-7.15 (m, 1H), 6.71 (s, 1H), 6.01-5.88 (m, 1H), 5.64 (s, 1H), 4.63-4.50 (m, 1H), 3.40-3.33 (m, 1H), 3.32-3.24 (m, 1H), 3.20-3.11 (m, 1H), 3.07-3.00 (m, 1H), 2.76 (d, J=4.6 Hz, 3H), 2.72 (s, 3H), 2.40 (s, 3H), 2.26-2.10 (m, 1H), 1.93-1.80 (m, 1H). LCMS^{AA} m/z=272 [M+H]⁺

### Example 257: N⁴,3,5,7-tetramethyl-N²-(1-methylpiperidin-4-yl)-1,8-naphthyridine-2,4-diamine

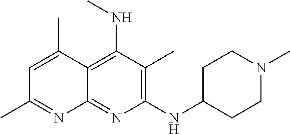

### Preparation 158—3,5,7-trimethyl-1,8-naphthyridine-2,4-diol

To a stirred solution of ethyl 2-amino-4,6-dimethylnicotinate (14.0 g, 72.2 mmol) and ethyl propionate (174 mL, 1.80 mol) in THF (200 mL) at room temperature was added t-BuOK (48.5 g, 433 mmol). The resulting mixture was stirred at room temperature for 40 min then was stirred at 100° C. for 3 h. The mixture was allowed to cool down to room temperature and concentrated under reduced pressure. The resulting mixture was diluted with water (150 mL). The residue was basified to pH 7 with HCl (aq.). The precipitated solids were collected by filtration and washed with water (3×10 mL). This affords the title compound as off-white solid (5.70 g, 38.7%). LCMS m/z=205 [M+H]⁺.

### Preparation 159—2,4-dichloro-3,5,7-trimethyl-1,8-naphthyridine

To a stirred solution of 3,5,7-trimethyl-1,8-naphthyridine-2,4-diol (5.70 g, 27.9 mmol) and DIEA (14.6 mL, 83.7 mmol) in toluene (50.0 mL) at 0° C. was added POCl₃ (26.0 mL, 279 mmol). The resulting mixture was stirred at 100° C. for 1 h. The mixture was allowed to cool down to room temperature. The reaction was quenched at 0° C. by the addition of sat. NaHCO₃ (aq.) (300 mL). The mixture was stirred at 0° C. for 15 min. The resulting mixture was extracted with EtOAc (3×400 mL). The combined organic layers were washed with brine (200 mL), dried over Na₂SO₄. After filtration, the filtrate was concentrated under reduce pressure to afford the title compound as a reddish solid (6.00 g, crude). LCMS m/z=241 [M+H]⁺.

### Preparation 160—4-chloro-3,5,7-trimethyl-N-(1-methylpiperidin-4-yl)-1,8-naphthyridin-2-amine

To a stirred solution of 2,4-dichloro-3,5,7-trimethyl-1,8-naphthyridine (1.00 g, 4.14 mmol) and 1-methylpiperidin-4-amine (947 mg, 8.29 mmol) in dioxane (10.0 mL) at room temperature was added CsF (189 mg, 12.4 mmol). The resulting mixture was stirred at 100° C. overnight. The mixture was allowed to cool down to room temperature and concentrated under reduced pressure. The residue was purified by prep-HPLC^{AV} to afford the title compound as white solid (410 mg, 31.0%). LCMS m/z=319 [M+H]⁺.

### Preparation 161—N⁴,3,5,7-tetramethyl-N²-(1-methylpiperidin-4-yl)-1,8-naphthyridine-2,4-diamine

To a stirred solution of 4-chloro-3,5,7-trimethyl-N-(1-methylpiperidin-4-yl)-1,8-naphthyridin-2-amine (200 mg, 0.648 mmol) and methanamine hydrochloride (105 mg, 1.29 mmol) in dioxane (2.00 mL) was added Pd(OAc)₂ (29.0 mg, 0.130 mmol) and Cs₂CO₃ (422 mg, 1.29 mmol) at room temperature. The resulting mixture was stirred under nitrogen atmosphere at 100° C. for overnight. The mixture was allowed to cool down to room temperature and concentrated under reduced pressure. The residue was purified by prep-HPLC^{AU} to afford the title compound as yellow solid (27.3 mg, 13.5%). ¹H NMR (400 MHz, DMSO-d₆) δ 6.71 (s, 1H), 5.90 (d, J=7.8 Hz, 1H), 4.59 (q, J=5.8 Hz, 1H), 4.14-4.02 (m, 1H), 2.84-2.75 (m, 2H), 2.74 (s, 3H), 2.66 (d, J=5.7 Hz, 3H), 2.41 (s, 3H), 2.18 (s, 3H), 2.08 (s, 3H), 2.02-1.94 (m, 2H), 1.89-1.81 (m, 2H), 1.66-1.52 (m, 2H). LCMS^{AA} m/z=314 [M+H]⁺.

### Example 258: (R)—N⁴,5-dimethyl-N²-(1-methylpyrrolidin-3-yl)-7,8-dihydro-6H-cyclopenta[b][1,8]naphthyridine-2,4-diamine

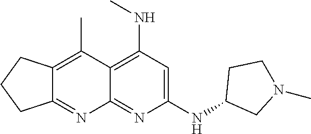 [SMILES: CNc1cc(N[C@@H]2CCN(C)C2)nc2nc3c(c(C)c12)CCC3]

### Preparation 162—5-methyl-7,8-dihydro-6H-cyclopenta[b][1,8]naphthyridine-2,4-diol

To a stirred solution of ethyl 2-amino-4-methyl-5H,6H,7H-cyclopenta[b]pyridine-3-carboxylate (3.50 g, 15.9 mmol) in ethyl acetate (175 mL) was added NaH (4.50 g, 113 mmol, 60% wt in oil) at room temperature. The resulting mixture was stirred at 80° C. for 5 h. The mixture was allowed to cool down to room temperature. The reaction was quenched by the addition of Water/Ice (200 mL) at 0° C. The aqueous layer was extracted with EtOAc (3×100 mL). The aqueous layer was acidified to pH 6 with AcOH. The precipitated solids were collected by filtration and washed with H₂O (3×5 mL). The resulting solid was dried under vacuum to afford the title compound as a grey solid (1.50 g, 43.7%). LCMS m/z=217 [M+H]⁺

### Preparation 163—2,4-dichloro-5-methyl-7,8-dihydro-6H-cyclopenta[b][1,8]naphthyridine

A solution of 5-methyl-6H,7H,8H-cyclopenta[b]1,8-naphthyridine-2,4-diol (1.40 g, 6.47 mmol) in POCl₃ (28.0 mL) was stirred at 100° C. for 1 h. The mixture was allowed to cool down to room temperature and concentrated under reduced pressure. The resulting mixture was diluted with NaHCO₃(aq.) (200 mL) and extracted with EtOAc (3×100 mL). The combined organic layers were washed with brine, dried over anhydrous Na₂SO₄. After filtration, the filtrate was concentrated under reduced pressure to afford the title compound as a tan solid (1.50 g, crude). LCMS m/z=253 [M+H]⁺

### Preparation 164—2-chloro-N,5-dimethyl-7,8-dihydro-6H-cyclopenta[b][1,8]naphthyridin-4-amine

To a stirred mixture of 2,4-dichloro-5-methyl-6H,7H,8H-cyclopenta[b]1,8-naphthyridine (1.30 g, 5.14 mmol) and methylamine hydrochloride (413 mg, 6.16 mmol) in DMSO (26.0 mL) was added DIEA (1.33 g, 10.3 mmol) at room temperature. The resulting mixture was stirred at 100° C. for 4 h. The mixture was allowed to cool down to room temperature. The resulting mixture was filtered, the filter cake was washed with DMSO (2×1 mL). The filtrate was purified by Prep-HPLC^{L} to afford the title compound as a grey solid (540 mg, 42.4%). LCMS m/z=248 [M+H]⁺

### Preparation 165—(R)—N⁴,5-dimethyl-N²-(1-methylpyrrolidin-3-yl)-7,8-dihydro-6H-cyclopenta[b][1,8]naphthyridine-2,4-diamine

To a stirred solution of 2-chloro-N,5-dimethyl-6H,7H,8H-cyclopenta[b]1,8-naphthyridin-4-amine (100 mg, 0.404 mmol) and (3R)-1-methylpyrrolidin-3-amine (65.0 μL, 0.606 mmol) in 1,4-dioxane (2.00 mL) were added Pd-PEPPSI-IPentCl 2-methylpyridine (o-picoline) (6.79 mg, 0.008 mmol) and Cs₂CO₃ (263 mg, 0.808 mmol) at room temperature. The resulting mixture was stirred under nitrogen atmosphere at 100° C. for 3 h. The mixture was allowed to cool down to room temperature and filtered, the filter cake was washed with MeOH (3×1 mL). The filtrate was concentrated under reduced pressure. The residue was purified Prep-HPLC^{L} to afford crude product (60 mg). The crude product was further purified by Prep-HPLC^{AU} to afford the title compound as a yellow solid (29.1 mg, 23.2%, ee>99%). ¹H NMR (400 MHz, Methanol-d₄) δ 5.71 (s, 1H), 4.70-4.59 (m, 1H), 3.03-2.91 (m, 5H), 2.88 (s, 3H), 2.81-2.76 (m, 1H), 2.70 (s, 3H), 2.65-2.49 (m, 2H), 2.48-2.33 (m, 4H), 2.26-2.07 (m, 2H), 1.82-1.70 (m, 1H). LCMS^{AB} m/z=312 [M+H]⁺.

### Example 259: N²-(3-azabicyclo[3.1.0]hexan-1-yl)-5,7-dimethylpyrido[2,3-d]pyrimidine-2,4-diamine, Isomer 1

### Example 260: N²-(3-azabicyclo[3.1.0]hexan-1-yl)-5,7-dimethylpyrido[2,3-d]pyrimidine-2,4-diamine, Isomer 2

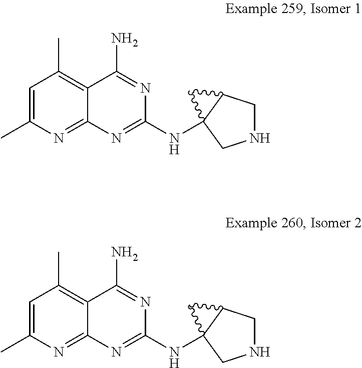

### Preparation 166—tert-butyl 1-((4-amino-5,7-dimethylpyrido[2,3-d]pyrimidin-2-yl)amino)-3-azabicyclo[3.1.0]hexane-3-carboxylate

A mixture of 2-chloro-5,7-dimethylpyrido[2,3-d]pyrimidin-4-amine (500 mg, 2.40 mmol) and tert-butyl 1-amino-3-azabicyclo[3.1.0]hexane-3-carboxylate (950 mg, 4.79 mmol) in DMSO (8.00 mL) was stirred at 100° C. overnight. The mixture was allowed to cool down to room temperature. The residue was purified by Prep-HPLC^{AV} to afford the title compound as a yellow solid (750 mg, 84.5%). LCMS m/z=371 [M+H]⁺.

### Preparation 167—N²-((1S,5R)-3-azabicyclo[3.1.0]hexan-1-yl)-5,7-dimethylpyrido[2,3-d]pyrimidine-2,4-diamine and N²-((1R,5S)-3-azabicyclo[3.1.0]hexan-1-yl)-5,7-dimethylpyrido[2,3-d]pyrimidine-2,4-diamine

A solution of tert-butyl 1-((4-amino-5,7-dimethylpyrido[2,3-d]pyrimidin-2-yl)amino)-3-azabicyclo[3.1.0]hexane-3-carboxylate (750 mg, 2.03 mmol) in 4.0 M HCl in 1,4-dioxane (8.0 mL) was stirred at room temperature for 1 h. The resulting mixture was concentrated under reduced pressure and dissolved in MeOH (1.00 mL). The mixture was basified to pH 9 with NH₃·H₂O. The residue was purified by Prep-HPLC^{AV} to afford the title compound as a white solid (500 mg, 91.3%) and then separated by Chiral-HPLC^{C} to afford Example 259 and Example 260 as off-white solids. The stereochemistry of Example 259 and Example 260 has not been unambiguously confirmed.

Example 259, Isomer 1: (51.3 mg, 12.8%, ee>99%, Rt=9.1 min). ¹H NMR (400 MHz, DMSO-d₆) δ 6.98 (s, 1H), 6.88-6.52 (m, 3H), 3.43-3.37 (m, 1H), 3.11 (d, J=10.5 Hz, 2H), 2.88 (d, J=10.5 Hz, 1H), 2.75 (d, J=11.0 Hz, 1H), 2.65 (s, 3H), 2.39 (s, 3H), 1.48 (dt, J=8.4, 4.1 Hz, 1H), 0.92 (t, J=5.0 Hz, 1H), 0.82-0.73 (m, 1H). LCMS^{T} m/z=271 [M+H]⁺

Example 260, Isomer 2: (44.9 mg, 11.2%, ee>99%, Rt=14.1 min). ¹H NMR (400 MHz, DMSO-d6) δ 7.08-6.84 (m, 1H), 6.80-6.50 (m, 3H), 3.42-3.27 (m, 1H), 3.08 (d, J=10.8 Hz, 2H), 2.80 (d, J=10.4 Hz, 1H), 2.74-2.59 (m, 4H), 2.39 (s, 3H), 1.51-1.39 (m, 1H), 0.94-0.85 (m, 1H), 0.76-0.68 (m, 1H). LCMS^{T} m/z=271 [M+H]⁺.

### Example 261: N⁴,5,7-trimethyl-N²-(1-methylpiperidin-4-yl)-1,8-naphthyridine-2,4-diamine

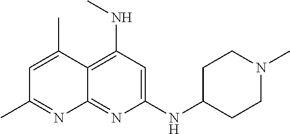

### Preparation 168—2-chloro-5,7-dimethyl-N-(1-methylpiperidin-4-y)-1,8-naphthyridin-4-amine and 4-chloro-5,7-dimethyl-N-(1-methylpiperidin-4-yl)-1,8-naphthyridin-2-amine

A solution of 2,4-dichloro-5,7-dimethyl-1,8-naphthyridine (1.00 g, 4.40 mmol) and 1-methylpiperidin-4-amine (1.51 g, 13.2 mmol) in DMSO (10.0 mL) was stirred at 100° C. for 3 h. The mixture was allowed to cool down to room temperature. The residue was purified by HPLC^{AV} to afford the title compound 2-chloro-5,7-dimethyl-N-(1-methylpiperidin-4-yl)-1,8-naphthyridin-4-amine (550 mg, 41.0%) as a yellow solid and 4-chloro-5,7-dimethyl-N-(1-methylpiperidin-4-yl)-1,8-naphthyridin-2-amine (530 mg, 39.5%) as a yellow solid. LCMS m/z=305 [M+H]⁺.

### Preparation 169—N⁴,5,7-trimethyl-N²-(1-methylpiperidin-4-yl)-1,8-naphthyridine-2,4-diamine

A mixture of 4-chloro-5,7-dimethyl-N-(1-methylpiperidin-4-yl)-1,8-naphthyridin-2-amine (200 mg, 0.656 mmol), methylamine hydrochloride (222 mg, 3.28 mmol), BrettPhos Pd G₃ (59.5 mg, 0.066 mmol), BrettPhos (70.4 mg, 0.131 mmol) and Cs₂CO₃ (278 mg, 2.62 mmol) in DMF (5.00 mL) was stirred under nitrogen atmosphere at 85° C. for 1 h. The mixture was allowed to cool down to room temperature. The residue was purified by HPLC^{BD} to afford the title compound as a white solid (38.9 mg, 19.8%). ¹H NMR (400 MHz, DMSO-d₆) δ 6.62 (s, 1H), 6.53 (d, J=8.0 Hz, 1H), 5.83-5.76 (m, 1H), 5.64 (s, 1H), 3.94-3.81 (m, 1H), 2.78-2.70 (m, 5H), 2.69 (s, 3H), 2.37 (s, 3H), 2.17 (s, 3H), 1.99 (t, J=11.6 Hz, 2H), 1.90-1.81 (m, 2H), 1.49-1.36 (in, 2H). LCMS^{T} m/z=300 [M+H]⁺.

### Example 262-Example 424

These Examples were synthesised according to methods similar to those cited in the following table:

| | | Method | | LCMS |
|---|---|---|---|---|
| Example | Structure | Ref | ¹H NMR | m/z |
| Example 262 | 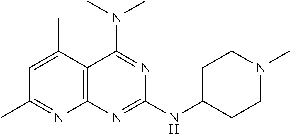 | Example 5 (Route B), using appropriate amine | ¹H NMR (400 MHz, DMSO-d₆) δ 6.85- 6.75 (m, 1H), 6.71 (s, 1H), 3.83-3.69 (m, 1H), 2.89 (s, 6H), 2.75 (d, J = 11.5 Hz, 2H), 2.50 (s, 3H), 2.39 (s, 3H), 2.16 (s, 3H), 1.94 (t, J = 11.5 Hz, 2H), 1.85-1.69 (m, 2H), 1.61-1.43 (m, 2H). | J: 315 [M + H]⁺ |
| Example 263 | 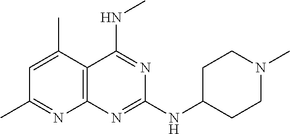 | Example 5 (Route B), using appropriate amine | ¹H NMR (400 MHz, DMSO-d₆) δ 6.74- 6.63 (m, 2H), 6.56-6.48 (m, 1H), 3.83- 3.70 (m, 1H), 2.93 (d, J = 4.4 Hz, 3H), 2.75 (d, J = 11.5 Hz, 2H), 2.64 (s, 3H), 2.37 (s, 3H), 2.16 (s, 3H), 2.00-1.89 (m, 2H), 1.86- 1.77 (m, 2H), 1.58-1.45 (m, 2H). | AH: 301 [M + H]⁺ |
| Example 264 | 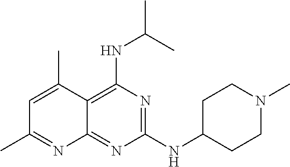 | Example 5 (Route B), using appropriate amine | ¹H NMR (400 MHz, DMSO-d₆) δ 6.66 (s, 1H), 6.60-6.42 (m, 1H), 6.30-6.05 (m, 1H), 4.47-4.21 (m, 1H), 3.81-3.68 (m, 1H), 2.75 (d, J = 11.2 Hz, 2H), 2.66 (s, 3H), 2.36 (s, 3H), 2.16 (s, 3H), 1.94 (t, J = 11.5 Hz, 2H), 1.81 (d, J = 12.3 Hz, 2H), 1.51 (q, J = 11.4, 10.7 Hz, 2H), 1.25 (d, J = 6.6 Hz, 6H). | B: 329 [M + H]⁺ |
| Example 265 | 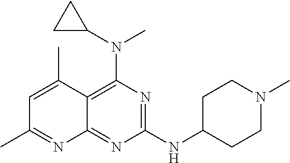 | Example 5 (Route B), using appropriate amine | ¹H NMR (400 MHz, DMSO-d₆) δ 6.95- 6.75 (m, 1H), 6.71 (s, 1H), 3.85-3.69 (m, 1H), 2.90 (s, 3H), 2.87-2.79 (m, 1H), 2.75 (d, J = 11.2 Hz, 2H), 2.50 (s, 3H), 2.40 (s, 3H), 2.16 (s, 3H), 1.94 (t, J = 11.5 Hz, 2H), 1.88-1.72 (m, 2H), 1.64-1.42 (m, 2H), 0.82- 0.57 (m, 2H), 0.55-0.14 (m, 2H). | AG: 341 [M + H]⁺ |
| Example 266 | 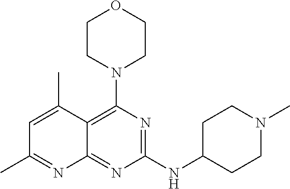 | Example 5 (Route B), using appropriate amine | ¹H NMR (400 MHz, DMSO-d₆) δ 6.95 (d, J = 7.6 Hz, 1H), 6.74 (s, 1H), 3.94-3.60 (m, 5H), 3.52-3.36 (m, 2H), 3.22-2.98 (m, 2H), 2.75 (d, J = 11.3 Hz, 2H), 2.58 (s, 3H), 2.41 (s, 3H), 2.16 (s, 3H), 2.00-1.88 (m, 2H), 1.82 (d, J = 12.4 Hz, 2H), 1.63- 1.43 (m, 2H). | AF: 357 [M + H]⁺ |
| Example 267 | 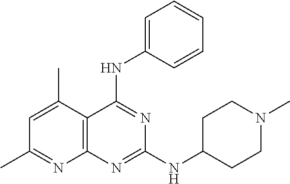 | Example 5 (Route B), using appropriate amine | ¹H NMR (400 MHz, DMSO-d₆) δ 8.54- 8.30 (m, 1H), 7.77 (d, J = 7.9 Hz, 2H), 7.34 (t, J = 7.7 Hz, 2H), 7.12-6.98 (m, 1H), 6.97-6.74 (m, 2H), 3.92-3.48 (m, 1H), 2.91-2.61 (m, 5H), 2.42 (s, 3H), 2.17 (s, 3H), 2.02-1.87 (m, 2H), 1.88-1.72 (m, 2H), 1.69-1.41 (m, 2H). | AG: 363 [M + H]⁺ |
| Example 268 | 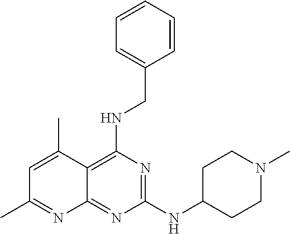 | Example 5 (Route B), using appropriate amine | ¹H NMR (400 MHz, DMSO-d₆) δ 7.63- 7.00 (m, 6H), 6.70 (s, 1H), 6.51 (d, J = 7.6 Hz, 1H), 4.72 (s, 2H), 3.87-3.49 (m, 1H), 2.87-2.61 (m, 5H), 2.38 (s, 3H), 2.15 (s, 3H), 2.02-1.71 (m, 3H), 1.64-1.24 (m, 3H). | E: 377 [M + H]⁺ |
| Example 269 | 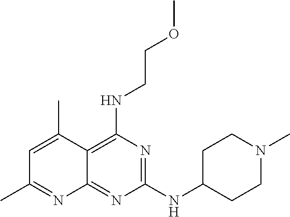 | Example 5 (Route B), using appropriate amine | ¹H NMR (400 MHz, DMSO-d₆) δ 6.68 (s, 1H), 6.65-6.49 (m, 2H), 3.87-3.70 (m, 1H), 3.68-3.60 (m, 2H), 3.59-3.52 (m, 2H), 3.31 (s, 3H), 2.76 (d, J = 11.4 Hz, 2H), 2.64 (s, 3H), 2.37 (s, 3H), 2.17 (s, 3H), 2.03-1.92 (m, 2H), 1.82 (d, J = 12.2 Hz, 2H), 1.60- 1.42 (m, 2H). | AF: 345 [M + H]⁺ |
| Example 270 | 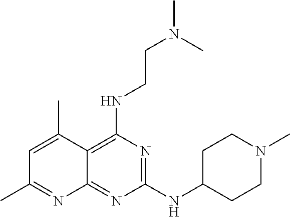 | Example 5 (Route B), using appropriate amine | ¹H NMR (400 MHz, DMSO-d₆) δ 6.87-6.62 (m, 2H), 6.50 (s, 1H), 3.76 (s, 1H), 3.52 (s, 2H), 2.75 (d, J = 10.9 Hz, 2H), 2.63 (s, 3H), 2.55-2.51 (m, 2H), 2.37 (s, 3H), 2.22 (s, 6H), 2.16 (s, 3H), 2.00-1.85 (m, 2H), 1.85- 1.73 (m, 2H), 1.61-1.44 (m, 2H). | AH: 358 [M + H]⁺ |
| Example 271 | 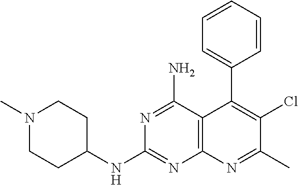 | Example 128, using Example 13 | ¹H NMR (400 MHz, DMSO-d₆) δ 7.72- 7.56 (m, 3H), 7.50-7.32 (m, 2H), 7.18- 6.80 (br, 2H), 4.02-3.87 (m, 1H), 3.18- 2.97 (m, 2H), 2.60 (s, 3H), 2.50-2.28 (m, 5H), 2.06-1.84 (m, 2H), 1.74-1.51 (m, 2H). | V: 383 [M + H]⁺ |
| Example 272 | 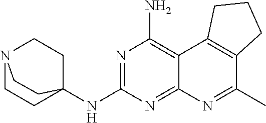 [SMILES: Cc1nc2nc(NC34CCN(CC3)CC4)nc(N)c2c2c1CCC2] | Example 11, using appropriate amine | ¹H NMR (400 MHz, DMSO-d₆) δ 6.54 (s, 2H), 5.74 (s, 1H), 2.98-2.69 (m, 10H), 2.39 (s, 3H), 2.17-2.04 (m, 2H), 2.01-1.87 (m, 6H). | AI: 325 [M + H]⁺ |
| Example 273 | 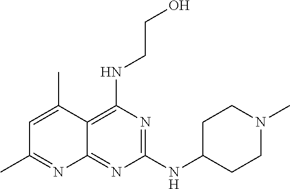 | Example 224, using Example 269 | ¹H NMR (400 MHz, DMSO-d₆) δ 6.68 (s, 1H), 6.64-6.44 (m, 2H), 4.82 (t, J = 5.1 Hz, 1H), 3.82-3.68 (m, 1H), 3.67-3.59 (m, 2H), 3.58-3.50 (m, 2H), 2.75 (d, J = 11.4 Hz, 2H), 2.66 (s, 3H), 2.37 (s, 3H), 2.16 (s, 3H), 1.94 (t, J = 11.5 Hz, 2H), 1.86-1.77 (m, 2H), 1.57-1.43 (m, 2H). | AH: 331 [M + H] |
| Example 274 | 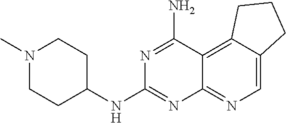 [SMILES: CN1CCC(Nc2nc(N)c3c4c(cnc3n2)CCC4)CC1] | Example 4, using appropriate starting material | ¹H NMR (400 MHz, DMSO-d₆) δ 8.47 (s, 1H), 6.69 (s, 2H), 6.41 (s, 1H), 3.86-3.69 (m, 1H), 3.31 (t, J = 7.5 Hz, 2H), 2.86 (t, J = 7.6 Hz, 2H), 2.79 (d, J = 8.0 Hz, 2H), 2.20 (s, 3H), 2.16-1.95 (m, 4H), 1.83 (d, J = 8.4 Hz, 2H), 1.60-1.44 (m, 2H). | V: 299 [M + H]⁺ |
| Example 275 | 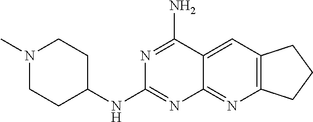 [SMILES: CN1CCC(Nc2nc(N)c3cc4c(nc3n2)CCC4)CC1] | Example 4, using appropriate starting material | ¹H NMR (400 MHz, DMSO-d₆) δ 8.13 (s, 1H), 7.19 (s, 2H), 6.36 (s, 1H), 3.82-3.72 (m, 1H), 2.94-2.82 (m, 4H), 2.75 (d, J = 11.5 Hz, 2H), 2.17 (s, 3H), 2.12-2.01 (m, 2H), 1.99-1.90 (m, 2H), 1.86-1.77 (m, 2H), 1.58-1.42 (m, 2H). | AH: 299 [M + H]⁺ |
| Example 276 | 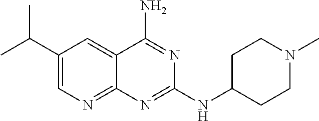 [SMILES: CC(C)c1cnc2nc(NC3CCN(C)CC3)nc(N)c2c1] | Example 245 (coupling), using Example 20 then reduction as per Example 246 | ¹H NMR (400 MHz, DMSO-d₆) δ 8.52 (s, 1H), 8.23 (s, 1H), 7.37 (br, 2H), 6.45 (s, 1H), 3.85-3.70 (m, 1H), 2.99-2.85 (m, 1H), 2.74 (d, J = 11.1 Hz, 2H), 2.16 (s, 3H), 1.93 (t, J = 11.7 Hz, 2H), 1.81 (d, J = 12.0 Hz, 2H), 1.57-1.42 (m, 2H), 1.24 (d, J = 6.9 Hz, 6H). | AJ: 301 [M + H]⁺ |
| Example 277 | 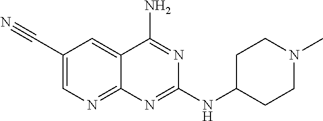 [SMILES: CN1CCC(Nc2nc(N)c3cc(C#N)cnc3n2)CC1] | Example 250, using Example 20 | ¹H NMR (400 MHz, DMSO-d₆) δ 8.93- 8.74 (m, 2H), 8.11-7.62 (m, 2H), 7.59-7.22 (m, 1H), 3.96-3.74 (m, 1H), 2.92-2.74 (m, 2H), 2.30-2.17 (m, 3H), 2.15-1.92 (m, 2H), 1.90-1.69 (m, 2H), 1.62-1.43 (m, 2H). | V: 284 [M + H]⁺ |
| Example 278 | 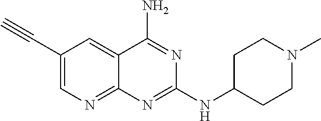 [SMILES: C#Cc1cnc2nc(NC3CCN(C)CC3)nc(N)c2c1] | Example 249, using Example 20 | ¹H NMR (400 MHz, DMSO-d₆) δ 8.65 (s, 1H), 8.57 (s, 1H), 7.95-7.31 (m, 2H), 7.29- 6.83 (m, 1H), 4.29 (s, 1H), 3.89 (s, 1H), 3.03-2.83 (m, 2H), 2.42-2.12 (m, 5H), 2.03- 1.80 (m, 2H), 1.70-1.48 (m, 2H). | AH: 283 [M + H]⁺ |
| Example 279 | 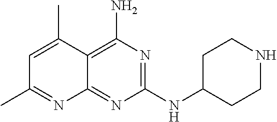 [SMILES: Cc1cc(C)c2c(N)nc(NC3CCNCC3)nc2n1] | Example 131, using appropriately protected amine, then deprotect per Example 140 | ¹H NMR (400 MHz, DMSO-d₆) δ 6.85- 6.53 (m, 3H), 6.37 (s, 1H), 4.03-3.79 (m, 1H), 2.94 (d, J = 12.1 Hz, 2H), 2.63 (s, 3H), 2.56-2.52 (m, 1H), 2.48-2.44 (m, 1H), 2.37 (s, 3H), 1.78 (d, J = 11.2 Hz, 2H), 1.40-1.26 (m, 2H). | AA: 273 [M + H]⁺ |
| Example 280 | 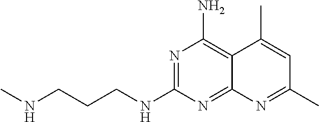 | Example 131, using appropriately protected amine, then deprotect per Example 140 | ¹H NMR (400 MHz, Methanol-d₄) δ 6.80 (s, 1H), 3.50 (t, J = 6.6 Hz, 2H), 3.30 (s, 3H), 2.71 (s, 3H), 2.67 (t, J = 6.6 Hz, 2H), 2.47 (s, 3H), 1.89-1.75 (m, 2H). | AF: 261 [M + H]⁺ |
| Example 281 | 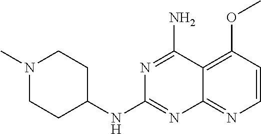 | Example 17, using appropriate starting material | ¹H NMR (400 MHz, DMSO-d₆) δ 8.40 (d, J = 5.5 Hz, 1H), 7.26 (s, 2H), 6.60 (s, 1H), 6.42 (s, 1H), 3.96 (s, 3H), 3.85-3.67 (m, 1H), 2.74 (d, J = 11.3 Hz, 2H), 2.16 (s, 3H), 1.93 (t, J = 11.5 Hz, 2H), 1.86-1.73 (m, 2H), 1.59-1.41 (m, 2H). | AK: 289 [M + H]⁺ |
| Example 282 | 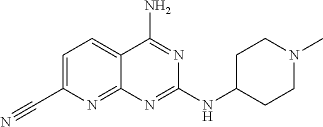 [SMILES: CN1CCC(Nc2nc(N)c3ccc(C#N)nc3n2)CC1] | Example 250, using Example 23 | ¹H NMR (400 MHz, DMSO-d₆) δ 8.65- 8.40 (m, 1H), 8.10-7.57 (m, 2H), 7.54 (d, J = 7.9 Hz, 1H), 7.04 (d, J = 8.1 Hz, 1H), 3.87-3.72 (m, 1H), 2.75 (d, J = 11.3 Hz, 2H), 2.16 (s, 3H), 1.94 (t, J = 11.8 Hz, 2H), 1.86-1.70 (m, 2H), 1.61-1.44 (m, 2H). | Z: 284 [M + H]⁺ |
| Example 283 | 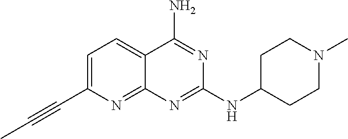 [SMILES: CC#Cc1ccc2c(N)nc(NC3CCN(C)CC3)nc2n1] | Example 251, using Example 23 | ¹H NMR (400 MHz, DMSO-d₆) δ 8.28 (d, J = 8.1 Hz, 1H), 7.76-7.26 (m, 2H), 7.02 (d, J = 8.4 Hz, 1H), 6.91-6.53 (m, 1H), 3.86- 3.67 (m, 1H), 2.75 (d, J = 11.1 Hz, 2H), 2.16 (s, 3H), 2.09 (s, 3H), 1.93 (t, J = 11.7 Hz, 2H), 1.87-1.71 (m, 2H), 1.61-1.40 (m, 2H). | C: 297 [M + H]⁺ |
| Example 284 | 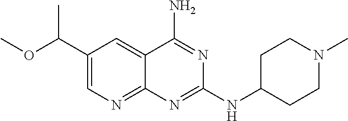 | Example 242, using Example 126 | ¹H NMR (400 MHz, DMSO-d₆) δ 8.55 (d, J = 2.4 Hz, 1H), 8.32 (d, J = 2.4 Hz, 1H), 7.42 (br, 2H), 6.55 (s, 1H), 4.43-4.30 (m, 1H), 3.87-3.72 (m, 1H), 3.13 (s, 3H), 2.75 (d, J = 11.2 Hz, 2H), 2.16 (s, 3H), 1.95 (t, J = 11.5 Hz, 2H), 1.81 (d, J = 12.3 Hz, 2H), 1.51 (qd, J = 12.1, 3.8 Hz, 2H), 1.42 (d, J = 6.5 Hz, 3H). | V: 317 [M + H]⁺ |
| Example 285 | 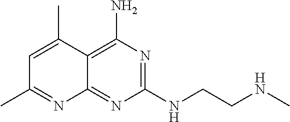 | Example 131, using appropriately protected amine, then deprotect per Example 140 | ¹H NMR (400 MHz, Methanol-d₄) δ 6.85 (s, 1H), 3.62 (t, J = 5.6 Hz, 2H), 3.09-2.96 (m, 2H), 2.72 (s, 3H), 2.58 (s, 3H), 2.48 (s, 3H). | V: 247 [M + H]⁺ |
| Example 286 | 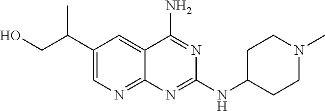 [SMILES: CC(CO)c1cnc2nc(NC3CCN(C)CC3)nc(N)c2c1] | Example 240, using Example 397 | ¹H NMR (400 MHz, DMSO-d₆) δ 8.49 (d, J = 2.3 Hz, 1H), 8.20 (d, J = 2.4 Hz, 1H), 7.37 (s, 2H), 6.46 (s, 1H), 4.77-4.65 (m, 1H), 3.90-3.71 (m, 1H), 3.53-3.46 (m, 2H), 2.84 (q, J = 6.9 Hz, 1H), 2.74 (d, J = 11.0 Hz, 2H), 2.16 (s, 3H), 2.07-1.90 (m, 2H), 1.81 (d, J = 12.3 Hz, 2H), 1.61-1.38 (m, 2H), 1.22 (d, J = 7.0 Hz, 3H). | AD: 317 [M + H]⁺ |
| Example 287 | 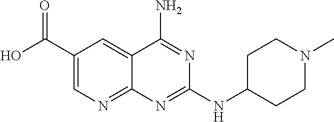 [SMILES: CN1CCC(Nc2nc(N)c3cc(C(=O)O)cnc3n2)CC1] | Example 236, using Example 122 | ¹H NMR (400 MHz, DMSO-d₆) δ 9.04 (d, J = 2.2 Hz, 1H), 8.83 (d, J = 2.4 Hz, 1H), 7.98- 7.08 (m, 2H), 6.73 (s, 1H), 3.91-3.76 (m, 1H), 2.77 (d, J = 11.1 Hz, 2H), 2.18 (s, 3H), 1.98 (t, J = 11.5 Hz, 2H), 1.82 (d, J = 12.1 Hz, 2H), 1.63-1.45 (m, 2H). | AA: 303 [M + H]⁺ |
| Example 288 | 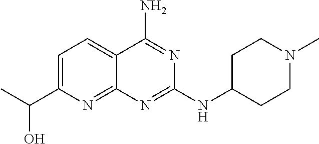 [SMILES: CC(O)c1ccc2c(N)nc(NC3CCN(C)CC3)nc2n1] | Example 126, using Example 289 | ¹H NMR (400 MHz, DMSO-d₆) δ 8.33 (d, J = 8.2 Hz, 1H), 7.60-7.20 (m, 2H), 7.14 (d, J = 8.1 Hz, 1H), 6.52 (s, 1H), 5.39 (s, 1H), 4.74-4.54 (m, 1H), 3.89-3.72 (m, 1H), 2.74 (d, J = 11.0 Hz, 2H), 2.16 (s, 3H), 1.95 (t, J = 11.6 Hz, 2H), 1.80 (d, J = 12.1 Hz, 2H), 1.61-1.43 (m, 2H), 1.36 (d, J = 6.6 Hz, 3H). | A: 303 [M + H]⁺ |
| Example 289 | 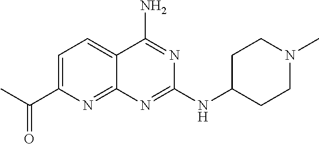 [SMILES: CC(=O)c1ccc2c(N)nc(NC3CCN(C)CC3)nc2n1] | Example 125, using Example 23 | ¹H NMR (400 MHz, DMSO-d₆) δ 8.50 (d, J = 8.2 Hz, 1H), 7.96-7.33 (m, 3H), 7.09-6.60 (m, 1H), 3.88 (s, 1H), 2.75 (d, J = 11.2 Hz, 2H), 2.65 (s, 3H), 2.17 (s, 3H), 2.09- 1.86 (m, 2H), 1.81 (d, J = 12.4 Hz, 2H), 1.67-1.44 (m, 2H). | A: 301 [M + H]⁺ |
| Example 290 | 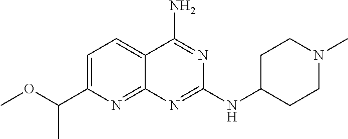 | Example 242, using Example 288 | ¹H NMR (400 MHz, DMSO-d₆) δ 8.36 (d, J = 8.2 Hz, 1H), 7.34 (s, 2H), 7.03 (d, J = 8.1 Hz, 1H), 6.55 (d, J = 6.7 Hz, 1H), 4.31 (q, J = 6.5 Hz, 1H), 3.91-3.72 (m, 1H), 3.21 (s, 3H), 2.75 (d, J = 11.2 Hz, 2H), 2.16 (s, 3H), 1.95 (t, J = 11.5 Hz, 2H), 1.80 (d, J = 12.2 Hz, 2H), 1.62-1.44 (m, 2H), 1.36 (d, J = 6.5 Hz, 3H). | AE: 317 [M + H]⁺ |
| Example 291 | 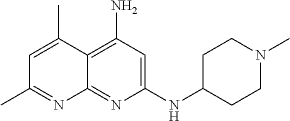 | Example 43, using appropriate starting material | ¹H NMR (400 MHz, DMSO-d₆) δ 6.61 (s, 1H), 6.41 (d, J = 7.8 Hz, 1H), 5.80 (s, 1H), 5.73 (s, 2H), 3.89-3.74 (m, 1H), 2.75 (d, J = 11.4 Hz, 2H), 2.69 (s, 3H), 2.37 (s, 3H), 2.17 (s, 3H), 1.97 (t, J = 11.5 Hz, 2H), 1.84 (d, J = 12.1 Hz, 2H), 1.57-1.37 (m, 2H). | AB: 286 [M + H]⁺ |
| Example 292 | 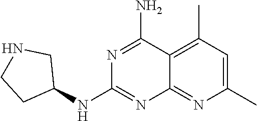 [SMILES: Cc1cc(C)c2c(N)nc(N[C@H]3CCNC3)nc2n1] | Example 131, using appropriately protected amine, then deprotect per Example 140 | ¹H NMR (400 MHz, DMSO-d₆) δ 7.00- 6.41 (m, 4H), 4.50-4.23 (m, 1H), 3.64- 3.49 (m, 1H), 2.99-2.82 (m, 2H), 2.81- 2.56 (m, 5H), 2.38 (s, 3H), 2.13-1.89 (m, 1H), 1.70-1.52 (m, 1H). | T: 259 [M + H]⁺ |
| Example 293 | 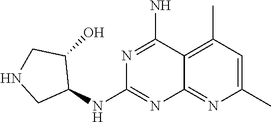 [SMILES: Cc1cc(C)c2c(N)nc(N[C@H]3CNC[C@@H]3O)nc2n1] | Example 131, using appropriately protected amine, then deprotect per Example 140 | ¹H NMR (400 MHz, DMSO-d₆) δ 6.99- 6.55 (m, 4H), 4.19-3.89 (m, 2H), 3.67- 3.55 (m, 1H), 3.19-3.07 (m, 1H), 3.09-3.00 (m, 1H), 2.74-2.63 (m, 4H), 2.59 (dd, J = 11.6, 3.7 Hz, 1H), 2.39 (s, 3H). | T: 275 [M + H]⁺ |
| Example 294 | 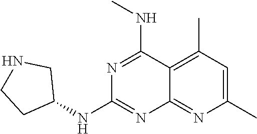 [SMILES: CNc1nc(N[C@@H]2CCNC2)nc2nc(C)cc(C)c12] | Example 263 then deprotect per Example 140 | ¹H NMR (400 MHz, Methanol-d₄) δ 6.80 (s, 1H), 4.58-4.50 (m, 1H), 3.30-3.23 (m, 2H), 3.14-2.90 (m, 5H), 2.72 (s, 3H), 2.47 (s, 3H), 2.34-2.19 (m, 1H), 2.00-1.89 (m, 1H). | T: 273 [M + H]⁺ |
| Example 295 | 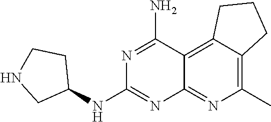 [SMILES: Cc1nc2nc(N[C@@H]3CCNC3)nc(N)c2c2c1CCC2] | Example 230, using other regioisomer and appropriately protected amine then deprotect per | ¹H NMR (400 MHz, Methanol-d₄) δ 4.58- 4.41 (m, 1H), 3.39-3.33 (m, 2H), 3.28-3.16 (m, 2H), 3.05-2.97 (m, 2H), 2.92 (t, J = 7.7 Hz, 2H), 2.48 (s, 3H), 2.30-2.14 (m, 3H), 1.96-1.81 (m, 1H). | AA: 285 [M + H]⁺ |
| | | Example 140 | | |
| Example 296 | 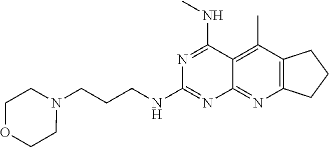 [SMILES: CNc1nc(NCCCN2CCOCC2)nc2nc3c(c(C)c12)CCC3] | Example 231, using appropriate amine | ¹H NMR (400 MHz, DMSO-d₆) δ 6.70 (s, 1H), 6.64 (t, J = 5.8 Hz, 1H), 3.57 (t, J = 4.6 Hz, 4H), 2.93 (d, J = 4.3 Hz, 3H), 2.89- 2.77 (m, 4H), 2.58 (s, 3H), 2.54-2.52 (m, 2H), 2.40-2.26 (m, 6H), 2.08-1.94 (m, 2H), 1.75-1.64 (m, 2H). | AA: 357 [M + H]⁺ |
| Example 297 | 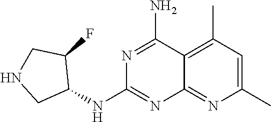 [SMILES: Cc1cc(C)c2c(N)nc(N[C@@H]3CNC[C@H]3F)nc2n1] | Example 131, using appropriately protected amine, then deprotect per Example 140 | ¹H NMR (400 MHz, DMSO-d₆) δ 7.09- 6.40 (m, 4H), 5.01 (dd, J = 53.4, 4.2 Hz, 1H), 4.38 (d, J = 20.5 Hz, 1H), 3.21-3.00 (m, 2H), 2.88 (dd, J = 26.5, 13.4 Hz, 1H), 2.74- 2.58 (m, 4H), 2.39 (s, 3H). | Z: 277 [M + H]⁺ |
| Example 298 | 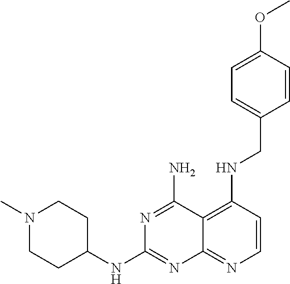 | Example 247, using appropriate amine | ¹H NMR (400 MHz, DMSO-d₆) δ 7.24 (d, J = 8.4 Hz, 2H), 7.00 (br, 2H), 6.89-6.85 (m, 2H), 6.30 (d, J = 8.2 Hz, 1H), 5.97 (d, J = 7.7 Hz, 1H), 4.29 (s, 2H), 3.73 (s, 3H), 3.71- 3.60 (m, 1H), 2.72 (d, J = 11.3 Hz, 2H), 2.14 (s, 3H), 1.97-1.85 (m, 2H), 1.76 (d, J = 11.8 Hz, 2H), 1.63-1.36 (m, 2H). | AA: 394 [M + H]⁺ |
| Example 299 | 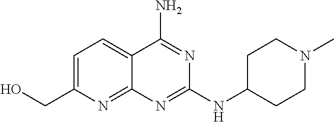 [SMILES: CN1CCC(Nc2nc(N)c3ccc(CO)nc3n2)CC1] | Example 123, using Example 235 | ¹H NMR (400 MHz, DMSO-d₆) δ 8.33 (d, J = 8.2 Hz, 1H), 7.31 (s, 2H), 7.10 (d, J = 8.1 Hz, 1H), 6.51 (s, 1H), 5.57-5.20 (m, 1H), 4.52 (s, 2H), 3.92-3.68 (m, 1H), 2.74 (d, J = 11.3 Hz, 2H), 2.16 (s, 3H), 1.94 (t, J = 11.4 Hz, 2H), 1.81 (d, J = 12.3 Hz, 2H), 1.59-1.44 (m, 2H). | AE: 289 [M + H]⁺ |
| Example 300 | 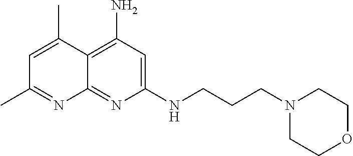 [SMILES: Cc1cc(C)c2c(N)cc(NCCCN3CCOCC3)nc2n1] | Example 256, using ammonia and appropriate amine | ¹H NMR (400 MHz, DMSO-d₆) δ 6.62 (s, 1H), 6.54 (t, J = 5.5 Hz, 1H), 5.83 (s, 1H), 5.74 (s, 2H), 3.58 (t, J = 4.6 Hz, 4H), 3.32- 3.24 (m, 2H), 2.69 (s, 3H), 2.42-2.23 (m, 9H), 1.79-1.59 (m, 2H). | Z: 316 [M + H]⁺ |
| Example 301 | 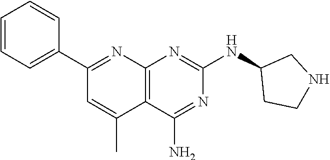 [SMILES: Cc1cc(-c2ccccc2)nc2nc(N[C@@H]3CCNC3)nc(N)c12] | Example 16, using appropriately protected amine, deprotection per Example 140 | ¹H NMR (400 MHz, Methanol-d₄) δ 8.21- 8.03 (m, 2H), 7.57-7.45 (m 3H), 7.45-7.40 (m, 1H), 4.62-4.51 (m, 1H), 3.30-3.21 (m, 2H), 3.14-2.99 (m, 2H), 2.84 (s, 3H), 2.35- 2.20 (m, 1H), 1.99-1.84 (m, 1H). | AB: 321 [M + H]⁺ |
| Example 302 | 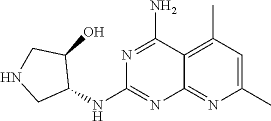 [SMILES: Cc1cc(C)c2c(N)nc(N[C@@H]3CNC[C@H]3O)nc2n1] | Example 131, using appropriately protected amine, then deprotect per Example 140 | ¹H NMR (400 MHz, DMSO-d₆) δ 7.08- 6.58 (m, 4H), 4.12 (s, 2H), 3.72-3.53 (m, 1H), 3.20-3.13 (m, 1H), 3.11-3.04 (m, 1H), 2.74 (d, J = 11.4 Hz, 1H), 2.70-2.59 (m, 4H), 2.39 (s, 3H). | V: 275 [M + H]⁺ |
| Example 303 | 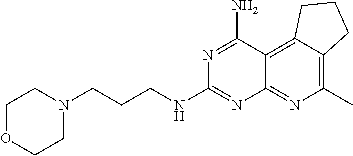 | Using alternative isomer described in Example 230 and method of Example 231 using appropriate amine | ¹H NMR (400 MHz, DMSO-d₆) δ 6.65 (t, J = 5.7 Hz, 1H), 6.49 (s, 1H), 3.58 (t, J = 4.6 Hz, 4H), 3.31-3.21 (m, 4H), 2.93 (d, J = 4.3 Hz, 3H), 2.81 (t, J = 7.6 Hz, 2H), 2.43-2.23 (m, 9H), 2.10 (p, J = 7.6 Hz, 2H), 1.70 (p, J = 7.1 Hz, 2H). | Z: 357 [M + H]⁺ |
| Example 304 | 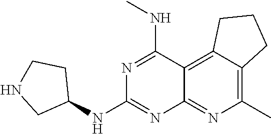 [SMILES: CNc1nc(N[C@@H]2CCNC2)nc2nc(C)c3c(c12)CCC3] | Using alternative isomer described in Example 230 and method of Example 231 using | ¹H NMR (400 MHz, Methanol-d₄) δ 4.57- 4.47 (m, 1H), 3.39-3.33 (m, 2H), 3.27-3.14 (m, 2H), 3.05 (s, 3H), 3.00-2.86 (m, 4H), 2.46 (s, 3H), 2.31-2.17 (m, 3H), 1.91-1.81 (m, 1H). | W: 299 [M + H]⁺ |
| | | appropriate | | |
| | | amine | | |
| Example 305 | 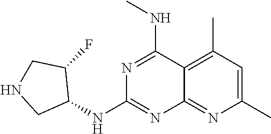 | Example 131, using appropriate protected amine, then deprotect per Example 140 | ¹H NMR (400 MHz, DMSO-d₆) δ 7.07- 6.65 (m, 3H), 6.29 (s, 1H), 5.09 (dt, J = 56.5, 4.3 Hz, 1H), 4.33 (d, J = 25.7 Hz, 1H), 3.25-3.16 (m, 1H), 3.10-3.03 (m, 1H), 2.95 (dd, J = 29.1, 13.6 Hz, 1H), 2.72 (t, J = 10.3 Hz, 1H), 2.65 (s, 3H), 2.39 (s, 3H). | T: 277 [M + H]⁺ |
| Example 306 | 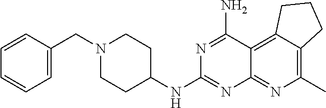 [SMILES: Cc1nc2nc(NC3CCN(Cc4ccccc4)CC3)nc(N)c2c2c1CCC2] | Example 46, using appropriate amine | ¹H NMR (400 MHz, DMSO-d₆) δ 7.41- 7.13 (m, 5H), 6.56 (s, 2H), 6.28 (s, 1H), 3.88-3.74 (m, 1H), 3.46 (s, 2H), 3.30-3.24 (m, 2H), 2.81 (t, J = 7.7 Hz, 4H), 2.37 (s, 3H), 2.14-1.94 (m, 4H), 1.88-1.76 (m, 2H), 1.58-1.45 (m, 2H). | Z: 389 [M + H]⁺ |
| Example 307 | 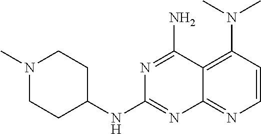 | Example 247, using appropriate amine | ¹H NMR (400 MHz, DMSO-d₆) δ 8.29 (d, J = 5.7 Hz, 1H), 8.21 (s, 1H), 7.82-7.04 (m, 2H), 6.67 (d, J = 5.6 Hz, 1H), 6.58 (s, 1H), 3.85- 3.77 (m, 1H), 2.94-2.69 (m, 8H), 2.22 (s, 3H), 2.13-1.96 (m, 2H), 1.82 (d, J = 12.5 Hz, 2H), 1.65-1.44 (m, 2H). | V: 302 [M + H]⁺ |
| Example 308 | 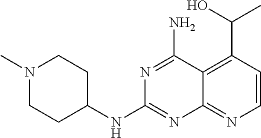 [SMILES: CC(O)c1ccnc2nc(NC3CCN(C)CC3)nc(N)c12] | Example 126, using Example 309 | ¹H NMR (400 MHz, Methanol-d₄) δ 8.64 (d, J = 4.7 Hz, 1H), 7.29 (s, 1H), 5.15 (q, J = 6.7 Hz, 1H), 4.29 (s, 1H), 3.53 (d, J = 12.8 Hz, 2H), 3.26-3.08 (m, 2H), 2.89 (s, 3H), 2.30 (d, J = 14.3 Hz, 2H), 2.01-1.82 (m, 2H), 1.60 (d, J = 6.7 Hz, 3H). | AE: 303 [M + H]⁺ |
| Example 309 | 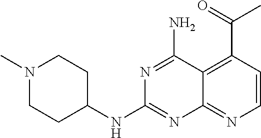 [SMILES: CC(=O)c1ccnc2nc(NC3CCN(C)CC3)nc(N)c12] | Example 125, using Example 245 | ¹H NMR (400 MHz, DMSO-d₆) δ 8.70- 8.56 (m, 1H), 7.24-6.93 (m, 2H), 6.89-6.50 (m, 2H) 3.86-3.77 (m, 1H), 2.78 (d, J = 11.3 Hz, 2H), 2.67 (s, 2H), 2.19 (s, 3H), 2.08-1.91 (m, 2H), 1.89-1.75 (m, 2H), 1.68-1.40 (m, 3H). | AE: 301 [M + H]⁺ |
| Example 310 | 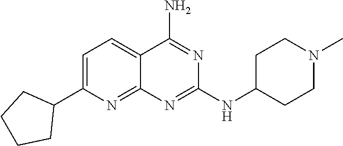 [SMILES: CN1CCC(Nc2nc(N)c3ccc(C4CCCC4)nc3n2)CC1] | Example 243, using Example 23 | ¹H NMR (400 MHz, DMSO-d₆) δ 8.22 (d, J = 8.2 Hz, 1H), 7.26 (br, 2H), 6.91 (d, J = 8.1 Hz, 1H), 6.44 (s, 1H), 3.81 (s, 1H), 3.21- 3.05 (m, 1H), 2.74 (d, J = 11.2 Hz, 2H), 2.16 (s, 3H), 2.02-1.86 (m, 4H), 1.86-1.69 (m, 6H), 1.67-1.58 (m, 2H), 1.58-1.43 (m, 2H). | V: 327 [M + H]⁺ |
| Example 311 | 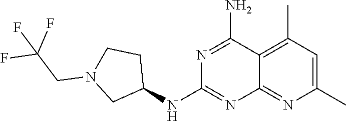 [SMILES: Cc1cc(C)c2c(N)nc(N[C@@H]3CCN(CC(F)(F)F)C3)nc2n1] | Example 142, using appropriate amine | ¹H NMR (400 MHz, DMSO-d₆) δ 6.92- 6.33 (m, 4H), 4.47-4.36 (m, 1H), 3.28-3.21 (m, 2H), 3.05 (t, J = 8.2 Hz, 1H), 2.88-2.78 (m, 1H), 2.72 (q, J = 7.8 Hz, 1H), 2.64 (s, 3H), 2.62-2.55 (m, 1H), 2.38 (s, 3H), 2.18- 2.06 (m, 1H), 1.85-1.70 (m, 1H). | R: 341 [M + H]⁺ |
| Example 312 | 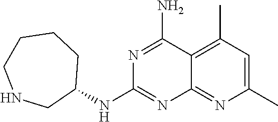 [SMILES: Cc1cc(C)c2c(N)nc(N[C@H]3CCCCNC3)nc2n1] | Example 131, using appropriately protected amine, then deprotect per Example 140 | ¹H NMR (400 MHz, Methanol-d₄) δ 6.80 (s, 1H), 4.26-4.12 (m, 1H), 3.24 (dd, J = 13.6, 3.9 Hz, 1H), 3.16-2.91 (m, 3H), 2.72 (s, 3H), 2.48 (s, 3H), 2.14-1.99 (m, 1H), 1.91-1.57 (m, 5H). | AA: 287 [M + H]⁺ |
| Example 313 | 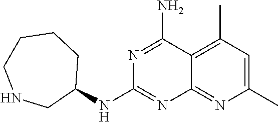 [SMILES: Cc1cc(C)c2c(N)nc(N[C@@H]3CCCCNC3)nc2n1] | Example 131, using appropriately protected amine, then deprotect per Example 140 | ¹H NMR (400 MHz, Methanol-d₄) δ 6.80 (s, 1H), 4.21-4.09 (m, 1H), 3.28-3.20 (m, 1H), 3.19-2.94 (m, 3H), 2.72 (s, 3H), 2.51- 2.41 (m, 3H), 2.14-2.01 (m, 1H), 1.88-1.74 (m, 3H), 1.71-1.53 (m, 2H). | W: 287 [M + H]⁺ |
| Example 314 | 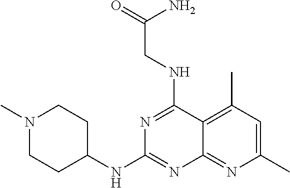 | Example 5 (Route B), using appropriate amine | ¹H NMR (400 MHz, DMSO-d₆) δ 7.48 (s, 1H), 7.19-6.87 (m, 2H), 6.71 (s, 1H), 6.63- 6.40 (m, 1H), 4.01 (s, 2H), 3.76 (s, 1H), 2.83-2.63 (m, 5H), 2.38 (s, 3H), 2.16 (s, 3H), 1.95 (t, J = 11.5 Hz, 2H), 1.81 (d, J = 12.1 Hz, 2H), 1.64-1.39 (m, 2H). | Y: 344 [M + H]⁺ |
| Example 315 | 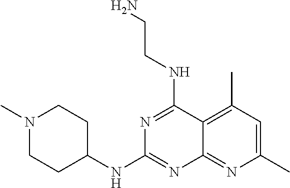 | Example 5 (Route B), using appropriately protected amine then deprotect per Example 140 | ¹H NMR (400 MHz, DMSO-d₆) δ 6.98- 6.43 (m, 3H), 3.82-3.73 (m, 1H), 3.31-3.19 (m, 2H), 2.89 (t, J = 6.0 Hz, 2H), 2.75 (d, J = 11.5 Hz, 2H), 2.67 (s, 3H), 2.37 (s, 3H), 2.16 (s, 3H), 2.03-1.89 (m, 2H), 1.89-1.73 (m, 2H), 1.61-1.39 (m, 2H). | AG: 330 [M + H]⁺ |
| Example 316 | 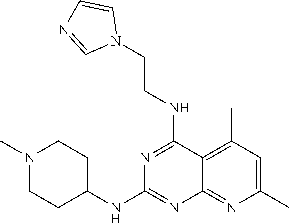 | Example 5 (Route B), using appropriate amine | ¹H NMR (400 MHz, Chloroform-d) δ 7.47 (s, 1H), 7.10 (s, 1H), 6.93 (s, 1H), 6.64 (s, 1H), 5.65 (s, 1H), 4.88 (s, 1H), 4.36-4.14 (m, 3H), 3.90-3.88 (m, 2H), 2.86 (d, J = 11.3 Hz, 2H), 2.54 (s, 3H), 2.45 (s, 3H), 2.34 (s, 3H), 2.23 (t, J = 11.5 Hz, 2H), 2.13 (d, J = 12.7 Hz, 2H), 1.63 (q, J = 11.5 Hz, 2H). | Y: 381 [M + H]⁺ |
| Example 317 | 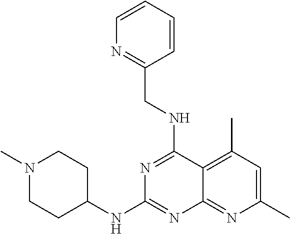 | Example 5 (Route B), using appropriate amine | ¹H NMR (400 MHz, DMSO-d₆) δ 8.55 (d, J = 4.8 Hz, 1H), 8.16 (s, 1H), 7.86-7.69 (m, 1H), 7.59 (s, 1H), 7.37 (d, J = 7.9 Hz, 1H), 7.28 (s, 1H), 7.02-6.76 (m, 2H), 4.83 (s, 2H), 4.01 (s, 1H), 3.11-2.90 (m, 3H), 2.86-2.58 (m, 7H), 2.42 (s, 3H), 2.23-1.44 (m, 4H). Formate salt | Y: 378 [M + H]⁺ |
| Example 318 | 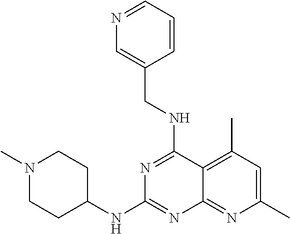 | Example 5 (Route B), using appropriate amine | ¹H NMR (400 MHz, Methanol-d₄) δ 8.60 (d, J = 2.3 Hz, 1H), 8.41 (dd, J = 4.9, 1.6 Hz, 1H), 7.87 (d, J = 7.9 Hz, 1H), 7.40 (dd, J = 7.9, 4.8 Hz, 1H), 6.87 (s, 1H), 4.84 (s, 2H), 4.18-3.69 (m, 1H), 3.25-3.01 (m, 2H), 2.81 (s, 3H), 2.72-2.52 (m, 5H), 2.49 (s, 3H), 2.25-2.18 (m, 2H), 1.75-1.47 (m, 2H). | AA: 378 [M + H] |
| Example 319 | 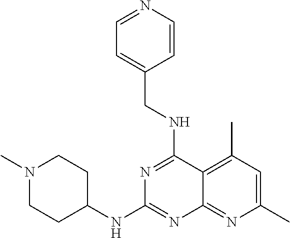 | Example 5 (Route B), using appropriate amine | ¹H NMR (400 MHz, DMSO-d₆) δ 8.47 (s, 2H), 7.59-7.16 (m, 3H), 6.72 (s, 1H), 6.60- 6.40 (m, 1H), 4.72 (s, 2H), 3.73 (s, 1H), 2.84-2.54 (m, 5H), 2.38 (s, 3H), 2.13 (s, 3H), 1.99-1.65 (m, 3H), 1.54-1.19 (m, 3H). | Y: 378 [M + H]⁺ |
| Example 320 | 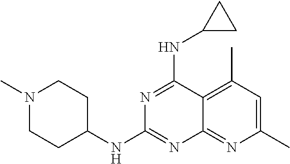 | Example 5 (Route B), using appropriate amine | ¹H NMR (400 MHz, DMSO-d₆) δ 6.92- 6.50 (m, 3H), 3.87-3.72 (m, 1H), 2.95- 2.81 (m, 1H), 2.76 (dt, J = 12.2, 3.5 Hz, 2H), 2.59 (s, 3H), 2.37 (s, 3H), 2.16 (s, 3H), 1.94 (td, J = 11.7, 2.4 Hz, 2H), 1.88- 1.74 (m, 2H), 1.62-1.46 (m, 2H), 0.83-0.72 (m, 2H), 0.66-0.56 (m, 2H). | W: 327 [M + H]⁺ |
| Example 321 | 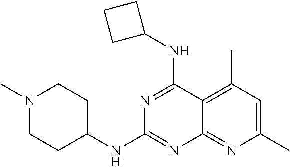 | Example 5 (Route B), using appropriate amine | ¹H NMR (400 MHz, DMSO-d₆) δ 6.80- 6.38 (m, 3H), 4.68-4.36 (m, 1H), 3.83-3.64 (m, 1H), 2.76 (dt, J = 12.1, 3.6 Hz, 2H), 2.68 (s, 3H), 2.42-2.28 (m, 5H), 2.17 (s, 3H), 2.07 (t, J = 10.3 Hz, 2H), 1.97 (dt, J = 12.7, 6.6 Hz, 2H), 1.90-1.61 (m, 4H), 1.59-1.41 (m, 2H). | W: 341 [M + H]⁺ |
| Example 322 | 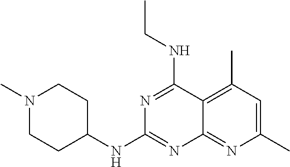 | Example 5 (Route B), using appropriate amine | ¹H NMR (400 MHz, DMSO-d₆) δ 6.66 (s, 1H), 6.60 (s, 1H), 6.53-6.41 (m, 1H), 3.83-3.71 (m, 1H), 3.48 (dt, J = 12.4, 6.2 Hz, 2H), 2.75 (d, J = 11.3 Hz, 2H), 2.65 (s, 3H), 2.36 (s, 3H), 2.16 (s, 3H), 2.04- 1.89 (m, 2H), 1.81 (d, J = 12.4 Hz, 2H), 1.51 (qd, J = 12.0, 3.7 Hz, 2H), 1.20 (t, J = 7.1 Hz, 3H). | W: 315 [M + H]⁺ |
| Example 323 | 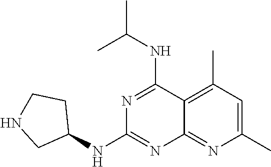 [SMILES: Cc1cc(C)c2c(NC(C)C)nc(N[C@@H]3CCNC3)nc2n1] | Example 5 (Route B), using appropriate amines, with deprotection as per Example 140 | ¹H NMR (400 MHz, Methanol-d₄) δ 6.78 (s, 1H), 4.57-4.35 (m, 2H), 3.26-3.13 (m, 2H), 3.06-2.92 (m, 2H), 2.73 (s, 3H), 2.46 (s, 3H), 2.31-2.19 (m, 1H), 1.96-1.83 (m, 1H), 1.32 (d, J = 6.5 Hz, 6H). | AK: 301 [M + H]⁺ |
| Example 324 | 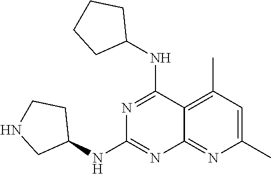 [SMILES: Cc1cc(C)c2c(NC3CCCC3)nc(N[C@@H]3CCNC3)nc2n1] | Example 5 (Route B), using appropriate amines, with deprotection as per Example 140 | ¹H NMR (400 MHz, Methanol-d₄) δ 6.80 (s, 1H), 4.56-4.44 (m, 2H), 3.29-3.19 (m, 2H), 3.10-2.97 (m, 2H), 2.73 (s, 3H), 2.47 (s, 3H), 2.29-2.17 (m, 1H), 2.17-2.07 (m, 1H), 2.17- 2.07 (m, 2H), 1.97-1.91 (m, 1H), 1.85-1.75 (m, 2H), 1.73-1.56 (m, 4H). | R: 327 [M + H]⁺ |
| Example 325 | 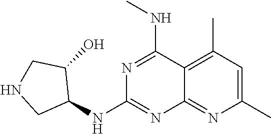 [SMILES: CNc1nc(N[C@H]2CNC[C@@H]2O)nc2nc(C)cc(C)c12] | Example 227, using appropriate amine | ¹H NMR (400 MHz, Methanol-d₄) δ 6.81 (s, 1H), 4.38-4.18 (m, 2H), 3.51-3.41 (m, 1H), 3.25-3.18 (m, 1H), 3.05 (s, 3H), 3.02-2.85 (m, 2H), 2.72 (s, 3H), 2.48 (s, 3H). | T: 289 [M + H]⁺ |
| Example 326 | 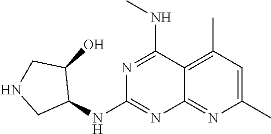 [SMILES: CNc1nc(N[C@H]2CNC[C@H]2O)nc2nc(C)cc(C)c12] | Example 227, using appropriate amine | ¹H NMR (400 MHz, Methanol-d₄) δ 6.80 (s, 1H), 4.53-4.44 (m, 1H), 4.42-4.28 (m, 1H), 3.41 (dd, J = 11.2, 7.6 Hz, 1H), 3.29-3.25 (m, 1H), 3.06 (s, 3H), 3.01 (dd, J = 12.4, 2.3 Hz, 1H), 2.91 (dd, J = 11.1, 8.7 Hz, 1H), 2.71 (s, 3H), 2.47 (s, 3H). | AA: 289 [M + H]⁺ |
| Example 327 | 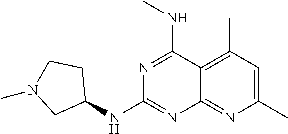 | Example 227, using appropriate amine | ¹H NMR (400 MHz, DMSO-d₆) δ 6.94- 6.51 (m, 3H), 4.51-4.31 (m, 1H), 3.00-2.88 (m, 3H), 2.81-2.72 (m, 1H), 2.64 (s, 3H), 2.55-2.52 (m, 1H), 2.46-2.30 (m, 5H), 2.24 (s, 3H), 2.20-2.06 (m, 1H), 1.77-1.63 (m, 1H). | R: 287 [M + H]⁺ |
| Example 328 | 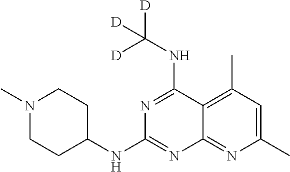 | Example 5 (Route B), using appropriate amine | ¹H NMR (400 MHz, DMSO-d₆) δ 6.80- 6.65 (m, 2H), 6.60 (s, 1H), 3.87-3.77 (m, 1H), 2.87 (d, J = 11.4 Hz, 2H), 2.64 (s, 3H), 2.37 (s, 3H), 2.27 (s, 3H), 2.22-2.10 (m, 2H), 1.87 (d, J = 12.4 Hz, 2H), 1.69-1.49 (m, 2H). | AA: 304 [M + H]⁺ |
| Example 329 | 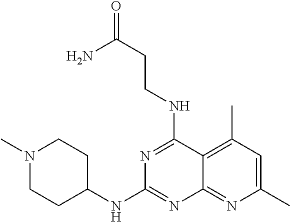 | Example 5 (Route B), using appropriate amine | ¹H NMR (400 MHz, DMSO-d₆) δ 7.43 (s, 1H), 6.93 (s, 2H), 6.67 (s, 1H), 6.55 (s, 1H), 3.76 (s, 1H), 3.65 (q, J = 6.3 Hz, 2H), 2.75 (d, J = 11.1 Hz, 2H), 2.63 (s, 3H), 2.45 (t, J = 6.6 Hz, 2H), 2.37 (s, 3H), 2.16 (s, 3H), 1.94 (td, J = 11.7, 2.4 Hz, 2H), 1.88-1.72 (m, 2H), 1.51 (qd, J = 12.0, 3.8 Hz, 2H). | S: 358 [M + H]⁺ |
| Example 330 | 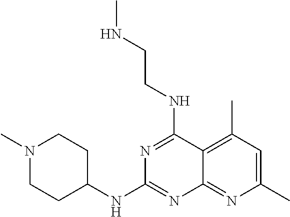 | Example 5 (Route B), using appropriately protected amine, then deprotect per Example 140 | ¹H NMR (400 MHz, DMSO-d₆) δ 6.83- 6.60 (m, 2H), 6.50 (s, 1H), 3.82-3.70 (m, 1H), 3.60-3.40 (m, 2H), 2.81-2.70 (m, 4H), 2.65 (s, 3H), 2.37 (s, 3H), 2.32 (s, 3H), 2.16 (s, 3H), 1.94 (td, J = 11.8, 2.5 Hz, 2H), 1.86-1.75 (m, 2H), 1.51 (qd, J = 12.0, 3.8 Hz, 2H). | AK: 344 [M + H]⁺ |
| Example 331 | 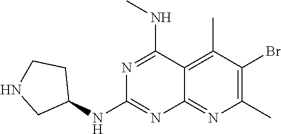 [SMILES: CNc1nc(N[C@@H]2CCNC2)nc2nc(C)c(Br)c(C)c12] | Example 130, using NBS, Example 131 using appropriate amines, deprotection as per | ¹H NMR (400 MHz, Methanol-d₄) δ 4.60- 4.48 (m, 1H), 3.26-3.19 (m, 1H), 3.18-3.10 (m, 1H), 3.06 (s, 3H), 3.03-2.86 (m, 2H), 2.84 (s, 3H), 2.67 (s, 3H), 2.29-2.15 (m, 1H), 1.92-1.78 (m, 1H). | AJ: 351 [M + H]⁺ |
| | | Example 140 | | |
| Example 332 | 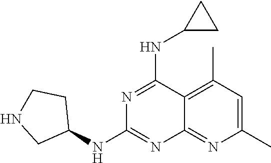 [SMILES: Cc1cc(C)c2c(NC3CC3)nc(N[C@@H]3CCNC3)nc2n1] | Example 227, using appropriate amine | ¹H NMR (400 MHz, Methanol-d₄) δ 6.77 (s, 1H), 4.61-4.42 (m, 1H), 3.36-3.31 (m, 1H), 3.27-3.21 (m, 1H), 3.19-2.98 (m, 2H), 2.97- 2.82 (m, 1H), 2.66 (s, 3H), 2.46 (s, 3H), 2.35- 2.18 (m, 1H), 2.02-1.87 (m, 1H), 0.96- 0.75 (m, 2H), 0.71-0.55 (m, 2H). | AJ: 299 [M + H]⁺ |
| Example 333 | 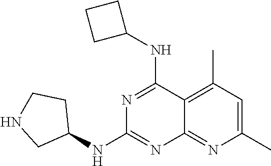 [SMILES: Cc1cc(C)c2c(NC3CCC3)nc(N[C@@H]3CCNC3)nc2n1] | Example 227, using appropriate amine | ¹H NMR (400 MHz, Methanol-d₄) δ 6.80 (s, 1H), 4.71-4.57 (m, 1H), 4.56-4.47 (m, 1H), 3.30-3.22 (m, 2H), 3.12-2.96 (m, 2H), 2.75 (s, 3H), 2.54-2.40 (m, 5H), 2.34-2.19 (m, 1H), 2.17-2.01 (m, 2H), 2.01-1.89 (m, 1H), 1.88-1.75 (m, 2H). | AB: 313 [M + H]⁺ |
| Example 334 | 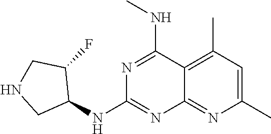 [SMILES: CNc1nc(N[C@H]2CNC[C@@H]2F)nc2nc(C)cc(C)c12] | Example 227, using appropriate amine | ¹H NMR (400 MHz, Methanol-d₄) δ 6.92 (s, 1H), 5.42 (dd, J = 50.5, 3.9 Hz, 1H), 4.66 (d, J = 17.3 Hz, 1H), 3.88-3.67 (m, 2H), 3.66- 3.49 (m, 2H), 3.09 (s, 3H), 2.76 (s, 3H), 2.52 (s, 3H). | Y: 291 [M + H]⁺ |
| Example 335 | 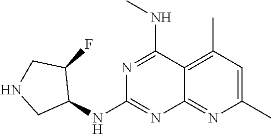 [SMILES: CNc1nc(N[C@H]2CNC[C@H]2F)nc2nc(C)cc(C)c12] | Example 227, using appropriate amine | ¹H NMR (400 MHz, Methanol-d₄) δ 6.81 (s, 1H), 5.19 (dt, J = 55.6, 3.9 Hz, 1H), 4.73- 4.55 (m, 1H), 3.47-3.32 (m, 2H), 3.27-3.11 (m, 1H), 3.07 (s, 3H), 2.81 (t, J = 10.6 Hz, 1H), 2.72 (s, 3H), 2.47 (s, 3H). | Y: 291 [M + H]⁺ |
| Example 336 | 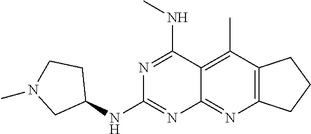 [SMILES: CNc1nc(N[C@@H]2CCN(C)C2)nc2nc3c(c(C)c12)CCC3] | Example 234, using appropriate amines | ¹H NMR (400 MHz, DMSO-d₆) δ 6.86-6.48 (m, 2H), 4.40 (s, 1H), 2.94 (d, J = 4.3 Hz, 3H), 2.91-2.76 (m, 5H), 2.66-2.53 (m, 4H), 2.48-2.43 (m, 1H), 2.41-2.33 (m, 1H), 2.26 (s, 3H), 2.21-2.09 (m, 1H), 2.02 (p, J = 7.7 Hz, 2H), 1.77-1.67 (m, 1H). | W: 313 [M + H]⁺ |
| Example 337 | 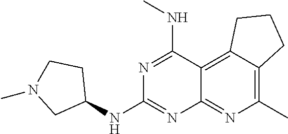 | Example 234, using appropriate amines | ¹H NMR (400 MHz, DMSO-d₆) δ 6.72-6.42 (m, 2H), 4.47-4.33 (m, 1H), 3.30-3.23 (m, 2H), 2.94 (d, J = 4.3 Hz, 3H), 2.86-2.73 (m, 3H), 2.57-2.52 (m, 1H), 2.43 (t, J = 7.5 Hz, 1H), 2.39-2.30 (m, 4H), 2.24 (s, 3H), 2.19-2.04 (m, 3H), 1.78-1.67 (m, 1H). | R: 313 [M + H]⁺ |
| Example 338 | 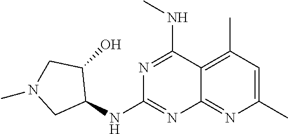 | Example 228, using appropriate amine | ¹H NMR (400 MHz, Methanol-d₄) δ 6.81 (s, 1H), 4.31-4.12 (m, 2H), 3.14 (t, J = 8.4 Hz, 1H), 3.09-2.96 (m, 4H), 2.71 (s, 3H), 2.68- 2.55 (m, 2H), 2.48 (s, 3H), 2.41 (s, 3H). | R: 303 [M + H]⁺ |
| Example 339 | 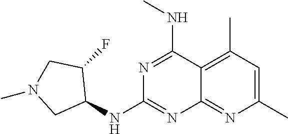 | Example 228, using appropriate amine | ¹H NMR (400 MHz, DMSO-d₆) δ 6.96 (s, 1H), 6.81 (s, 1H), 6.72 (s, 1H), 5.04 (d, J = 56.5 Hz, 1H), 4.71-4.39 (m, 1H), 3.18 (t, J = 8.3 Hz, 1H), 3.07-2.93 (m, 3H), 2.93-2.82 (m, 1H), 2.62 (s, 3H), 2.59-2.52 (m, 1H), 2.39 (s, 3H), 2.26 (s, 3H), 2.15 (t, J = 8.2 Hz, 1H). | U: 305 [M + H]⁺ |
| Example 340 | 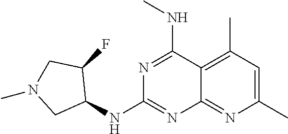 | Example 228, using appropriate amine | ¹H NMR (400 MHz, DMSO-d₆) δ 6.97- 6.76 (m, 1H), 6.73 (s, 1H), 6.63-6.50 (m, 1H), 5.14 (dtd, J = 56.2, 5.1, 2.0 Hz, 1H), 4.55-4.43 (m, 1H), 3.24-3.08 (m, 1H), 2.95 (d, J = 4.3 Hz, 3H), 2.88 (t, J = 8.1 Hz, 1H), 2.66 (s, 3H), 2.64-2.52 (m, 2H), 2.38 (s, 3H), 2.30 (s, 3H). | T: 305 [M + H]⁺ |
| Example 341 | 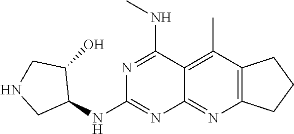 [SMILES: CNc1nc(N[C@H]2CNC[C@@H]2O)nc2nc3c(c(C)c12)CCC3] | Example 231, using appropriate amine | ¹H NMR (400 MHz, Methanol-d₄) δ 4.29- 4.12 (m, 2H), 3.45-3.37 (m, 1H), 3.21-3.14 (m, 1H), 3.05 (s, 3H), 3.02-2.91 (m, 4H), 2.89-2.81 (m, 2H), 2.64 (s, 3H), 2.13 (p, J = 7.6 Hz, 2H). | Y: 315 [M + H]⁺ |
| Example 342 | 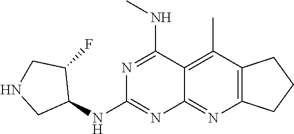 [SMILES: CNc1nc(N[C@H]2CNC[C@@H]2F)nc2nc3c(c(C)c12)CCC3] | Example 231, using appropriate amine | ¹H NMR (400 MHz, DMSO-d₆) δ 6.80 (s, 1H), 6.71 (s, 1H), 5.02 (dd, J = 53.2, 4.0 Hz 1H), 4.45-4.24 (m, 1H), 3.20-3.09 (m, 2H), 3.09-3.00 (m, 1H), 2.99-2.78 (m, 7H), 2.72 (dd, J = 11.8, 4.0 Hz, 1H), 2.59 (s, 3H), 2.03 (p, J = 7.6 Hz, 2H). | R: 317 [M + H]⁺ |
| Example 343 | 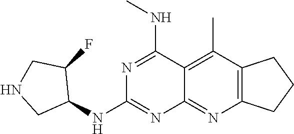 [SMILES: CNc1nc(N[C@H]2CNC[C@H]2F)nc2nc3c(c(C)c12)CCC3] | Example 231, using appropriate amine | ¹H NMR (400 MHz, Methanol-d₄) δ 5.19 (dt, J = 55.4, 4.0 Hz, 1H), 4.73-4.53 (m, 1H), 3.51-3.32 (m, 2H), 3.28-3.12 (m, 1H), 3.07 (s, 3H), 3.03-2.91 (m, 4H), 2.82 (t, J = 10.6 Hz, 1H), 2.66 (s, 3H), 2.14 (p, J = 7.7 Hz, 2H). | U: 317 [M + H]⁺ |
| Example 344 | 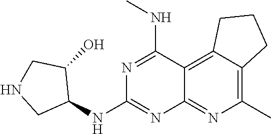 [SMILES: CNc1nc(N[C@H]2CNC[C@@H]2O)nc2nc(C)c3c(c12)CCC3] | Example 230, separating other regioisomer, then Example 231, using appropriate amine | ¹H NMR (400 MHz, Methanol-d₄) δ 4.42- 4.36 (m, 1H), 4.36-4.30 (m, 1H), 3.66-3.57 (m, 1H), 3.41 (dd, J = 12.4, 5.0 Hz, 1H), 3.36-3.32 (m, 2H), 3.22 (dd, J = 12.3, 3.7 Hz, 1H), 3.12-3.01 (m, 4H), 2.91 (t, J = 7.7 Hz, 2H), 2.48 (s, 3H), 2.25 (p, J = 7.7 Hz, 2H). | T: 315 [M + H]⁺ |
| Example 345 | 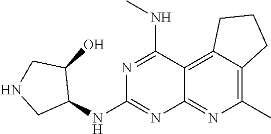 [SMILES: CNc1nc(N[C@H]2CNC[C@H]2O)nc2nc(C)c3c(c12)CCC3] | Example 230, separating other regioisomer, then Example 231, using appropriate amine | ¹H NMR (400 MHz, DMSO-d₆) δ 6.57 (s, 1H), 5.86 (d, J = 6.8 Hz, 1H), 5.08 (s, 1H), 4.21-4.06 (m, 2H), 3.31-3.27 (m, 2H), 3.61- 3.04 (m, 2H), 2.94 (d, J = 4.4 Hz, 3H), 2.82 (t, J = 7.6 Hz, 2H), 2.67 (dd, J = 11.9, 2.5 Hz, 1H), 2.57 (dd, J = 10.6, 7.7 Hz, 1H), 2.38 (s, 3H), 2.11 (p, J = 7.6 Hz, 2H). | S: 315 [M + H]⁺ |
| Example 346 | 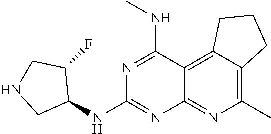 [SMILES: CNc1nc(N[C@H]2CNC[C@@H]2F)nc2nc(C)c3c(c12)CCC3] | Example 230, separating other regioisomer, then Example 231, using appropriate amine | ¹H NMR (400 MHz, Methanol-d₄) δ 5.26- 5.03 (m, 1H), 4.67-4.52 (m, 1H), 3.46-3.38 (m, 1H), 3.36-3.32 (m, 2H), 3.25-3.08 (m, 2H), 3.09-3.02 (m, 3H), 2.96-2.80 (m, 3H), 2.45 (s, 3H), 2.24 (p, J = 7.7 Hz, 2H). | W: 317 [M + H]⁺ |
| Example 347 | 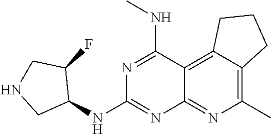 [SMILES: CNc1nc(N[C@H]2CNC[C@H]2F)nc2nc(C)c3c(c12)CCC3] | Example 230, separating other regioisomer, then Example 231, using appropriate amine | ¹H NMR (400 MHz, DMSO-d₆) δ 6.60 (s, 1H), 6.45 (s, 1H), 5.13 (dt, J = 56.3, 4.2 Hz, 1H), 4.39 (d, J = 26.4 Hz, 1H), 3.34-3.30 (m, 3H), 3.17-2.98 (m, 2H), 2.95 (d, J = 4.3 Hz, 3H), 2.89-2.75 (m, 3H), 2.38 (s, 3H), 2.12 (p, J = 7.6 Hz, 2H). | T: 317 [M + H]⁺ |
| Example 348 | 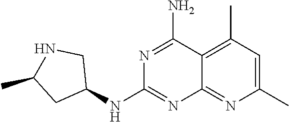 | Example 131, using appropriately protected amine, then deprotect per Example 140 | ¹H NMR (400 MHz, Methanol-d₄) δ 6.81 (s, 1H), 4.57-4.47 (m, 1H), 3.24-3.14 (m, 2H), 3.03-2.92 (m, 1H), 2.72 (s, 3H), 2.48 (s, 3H), 2.47-2.41 (m, 1H), 1.46-1.33 (m, 1H), 1.29 (d, J = 6.4 Hz, 3H). | T: 273 [M + H]⁺ |
| Example 349 | 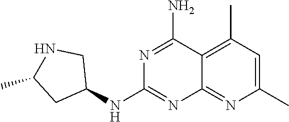 | Example 131, using appropriately protected amine, then deprotect per Example 140 | ¹H NMR (400 MHz, Methanol-d₄) δ 6.83 (s, 1H), 4.62-4.53 (m, 1H), 3.69-3.56 (m, 1H), 3.56-3.45 (m, 1H), 3.06-2.96 (m, 1H), 2.72 (s, 3H), 2.49 (s, 3H), 2.15-2.05 (m, 1H), 1.91-1.79 (m, 1H), 1.28 (d, J = 6.5 Hz, 3H). | T: 273 [M + H]⁺ |
| Example 350 | 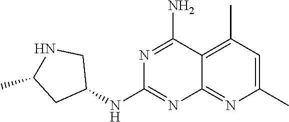 | Example 131, using appropriately protected amine, then deprotect per Example 140 | ¹H NMR (400 MHz, Methanol-d₄) δ 6.83 (s, 1H), 4.51 (qd, J = 7.7, 4.4 Hz, 1H), 3.35- 3.31 (m, 1 H), 3.29-3.24 (m, 1H), 3.17- 3.04 (m, 1H), 2.72 (s, 3H), 2.53-2.40 (m, 4H), 1.62-1.44 (m, 1H), 1.34 (d, J = 6.4 Hz, 3H). | T: 273 [M + H]⁺ |
| Example 351 | 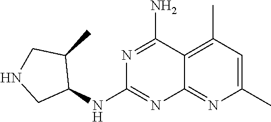 [SMILES: Cc1cc(C)c2c(N)nc(N[C@H]3CNC[C@H]3C)nc2n1] | Example 131, using appropriately protected amine, then deprotect per Example 140 | ¹H NMR (400 MHz, Methanol-d₄) δ 6.82 (s, 1H), 4.67-4.56 (m, 1H), 3.41-3.32 (m, 1H), 3.27-3.18 (m, 1H), 3.04 (d, J = 11.4 Hz, 1H), 2.83-2.74 (m, 1H), 2.72 (s, 3H), 2.52- 2.38 (m, 4H), 1.01 (d, J = 7.0 Hz, 3H). | S: 273 [M + H]⁺ |
| Example 352 | 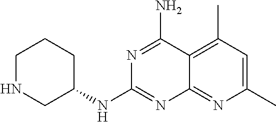 [SMILES: Cc1cc(C)c2c(N)nc(N[C@H]3CCCNC3)nc2n1] | Example 131, using appropriately protected amine, then deprotect per Example 140 | ¹H NMR (400 MHz, DMSO-d₆) δ 6.97-6.53 (m, 3H), 6.28 (s, 1H), 3.94-3.79 (m, 1H), 3.01 (d, J = 11.3 Hz, 1H), 2.78 (d, J = 11.7 Hz, 1H), 2.63 (s, 3H), 2.48-2.30 (m, 5H), 1.89-1.78 (m, 1H), 1.66-1.56 (m, 1H), 1.50- 1.33 (m, 2H). | U: 273 [M + H]⁺ |
| Example 353 | 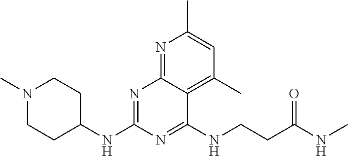 | Example 5 (Route B), using appropriate amine | ¹H NMR (400 MHz, DMSO-d₆) δ 7.89 (s, 1H), 6.96 (s, 1H), 6.67 (s, 1H), 6.60-6.47 (m, 1H), 3.84-3.72 (m, 1H), 3.66 (q, J = 6.3 Hz, 2H), 2.76 (d, J = 11.3 Hz, 2H), 2.63 (s, 3H), 2.58 (d, J = 4.6 Hz, 3H), 2.45 (t, J = 6.6 Hz, 2H), 2.37 (s, 3H), 2.17 (s, 3H), 2.04- 1.92 (m, 2H), 1.82 (d, J = 12.4 Hz, 2H), 1.57- 1.43 (m, 2H). | AK: 372 [M + H]⁺ |
| Example 354 | 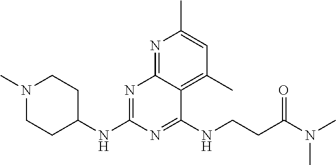 | Example 5 (Route B), using appropriate amine | ¹H NMR (400 MHz, DMSO-d₆) δ 6.96 (s, 1H), 6.67 (s, 1H), 6.53 (s, 1H), 3.83-3.63 (m, 3H), 2.95 (s, 3H), 2.84 (s, 3H), 2.75 (d, J = 11.5 Hz, 2H), 2.71-2.64 (m, 2H), 2.62 (s, 3H), 2.37 (s, 3H), 2.16 (s, 3H), 1.94 (t, J = 11.4 Hz, 2H), 1.82 (d, J = 12.3 Hz, 2H), 1.60-1.38 (m, 2H). | S: 386 [M + H]⁺ |
| Example 355 | 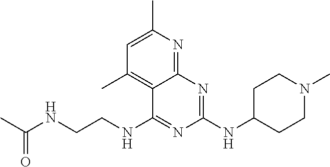 [SMILES: CC(=O)NCCNc1nc(NC2CCN(C)CC2)nc2nc(C)cc(C)c12] | Example 5 (Route B), using appropriate amine | ¹H NMR (400 MHz, Methanol-d₄) δ 6.78 (s, 1H), 4.07-3.94 (m, 1H), 3.69 (t, J = 6.0 Hz, 2H), 3.49 (t, J = 5.9 Hz, 2H), 2.93 (d, J = 11.7 Hz, 2H), 2.73 (s, 3H), 2.46 (s, 3H), 2.39-2.26 (m, 5H), 2.07 (d, J = 12.7 Hz, 2H), 1.93 (s, 3H), 1.69-1.55 (m, 2H). | U: 372 [M + H]⁺ |
| Example 356 | 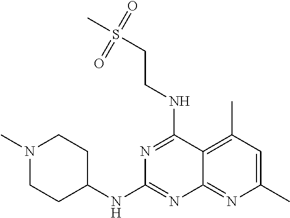 | Example 5 (Route B), using appropriate amine | ¹H NMR (400 MHz, DMSO-d₆) δ 6.94 (s, 1H), 6.69 (s, 1H), 6.63 (s, 1H), 3.88 (s, 2H), 3.81-3.71 (m, 1H), 3.51 (t, J = 6.7 Hz, 2H), 3.07 (s, 3H), 2.75 (d, J = 11.1 Hz, 2H), 2.65 (s, 3H), 2.38 (s, 3H), 2.16 (s, 3H), 1.96 (t, J = 11.5 Hz, 2H), 1.84 (d, J = 12.3 Hz, 2H), 1.51 (qd, J = 12.2, 3.8 Hz, 2H). | Y: 393 [M + H]⁺ |
| Example 357 | 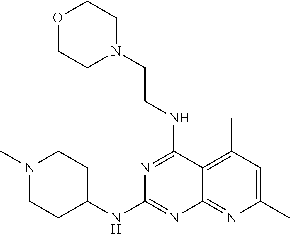 | Example 5 (Route B), using appropriate amine | ¹H NMR (400 MHz, DMSO-d₆) δ 6.82 (s, 1H), 6.69 (s, 1H), 6.51 (s, 1H), 3.83-3.70 (m, 1H), 3.63-3.51 (m, 6H), 2.75 (d, J = 11.2 Hz, 2H), 2.68 (s, 3H), 2.58 (t, J = 6.3 Hz, 2H), 2.48-2.41 (m, 4H), 2.37 (s, 3H), 2.16 (s, 3H), 1.99-1.88 (m, 2H), 1.88-1.74 (m, 2H), 1.51 (qd, J = 12.0, 3.8 Hz, 2H). | S: 400 [M + H]⁺ |
| Example 358 | 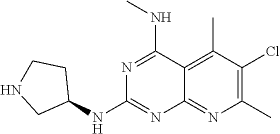 [SMILES: CNc1nc(N[C@@H]2CCNC2)nc2nc(C)c(Cl)c(C)c12] | Example 5 (Route B), using appropriately protected amines, chlorination as per Example 130, | ¹H NMR (400 MHz, Methanol-d₄) δ 4.60- 4.48 (m, 1H), 3.37-3.31 (m, 2H), 3.14- 2.99 (m, 5H), 2.81 (s, 3H), 2.62 (s, 3H), 2.35-2.18 (m, 1H), 2.01-1.88 (m, 1H). | W: 307 [M + H]⁺ |
| | | deprotection | | |
| | | as per | | |
| | | Example 140 | | |
| Example 359 | 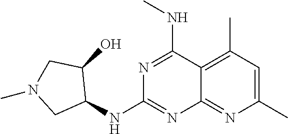 | Example 228, using appropriate amine | ¹H NMR (400 MHz, DMSO-d₆) δ 6.78 (s, 1H), 6.71 (s, 1H), 5.95 (s, 1H), 5.19 (s, 1H), 4.35-4.18 (m, 2H), 3.05-2.84 (m, 5H), 2.65 (s, 3H), 2.38 (s, 3H), 2.31-2.25 (m, 2H), 2.24 (s, 3H). | S: 303 [M + H]⁺ |
| Example 360 | 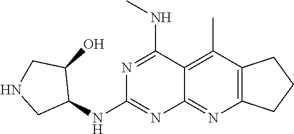 [SMILES: CNc1nc(N[C@H]2CNC[C@H]2O)nc2nc3c(c(C)c12)CCC3] | Example 231, using appropriate amine | ¹H NMR (400 MHz, Methanol-d₄) δ 4.53- 4.43 (m, 1H), 4.39-4.32 (m, 1H), 3.49-3.37 (m, 1H), 3.29-3.24 (m, 1H), 3.06 (s, 3H), 3.03-2.86 (m, 6H), 2.66-2.63 (m, 3H), 2.14 (p, J = 7.6 Hz, 2H). | AL: 315 [M + H]⁺ |
| Example 361 | 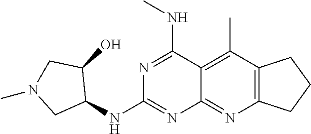 [SMILES: CNc1nc(N[C@H]2CN(C)C[C@H]2O)nc2nc3c(c(C)c12)CCC3] | Example 232, using appropriate amine | ¹H NMR (400 MHz, DMSO-d₆) δ 6.79 (s, 1H), 5.88 (s, 1H), 5.18 (s, 1H), 4.34-4.15 (m, 2H), 3.06-2.92 (m, 5H), 2.87 (q, J = 7.6 Hz, 4H), 2.59 (s, 3H), 2.31-2.19 (m, 5H), 2.02 (p, J = 7.7 Hz, 2H). | U: 329 [M + H]⁺ |
| Example 362 | 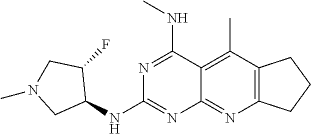 [SMILES: CNc1nc(N[C@H]2CN(C)C[C@@H]2F)nc2nc3c(c(C)c12)CCC3] | Example 233, using appropriate amine | ¹H NMR (400 MHz, Methanol-d₄) δ 5.12- 4.96 (m, 1H), 4.77-4.64 (m, 1H), 3.41-3.33 (m, 1H), 3.07 (s, 3H), 3.04-2.87 (m, 5H), 2.87-2.70 (m, 1H), 2.63 (s, 3H), 2.40 (s, 3H), 2.35 (t, J = 8.4 Hz, 1H), 2.12 (p, J = 7.7 Hz, 2H). | T: 331 [M + H]⁺ |
| Example 363 | 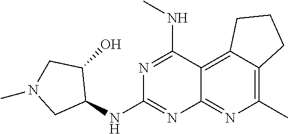 | Example 230, separating other regioisomer, then as per Example 232, using appropriate amine | ¹H NMR (400 MHz, Methanol-d₄) δ 4.31- 4.17 (m, 2H), 3.37-3.33 (m, 2H), 3.10 (t, J = 9.0 Hz, 1H), 3.05 (s, 3H), 2.97 (dd, J = 10.4, 6.5 Hz, 1H), 2.91 (t, J = 7.7 Hz, 2H), 2.64-2.53 (m, 2H), 2.47 (s, 3H), 2.38 (s, 3H), 2.25 (p, J = 7.7 Hz, 2H). | U: 329 [M + H]⁺ |
| Example 364 | 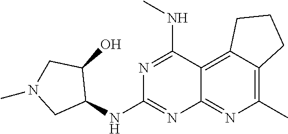 | Example 230, separating other regioisomer, then as per Example 232, using appropriate amine | ¹H NMR (400 MHz, Methanol-d₄) δ 4.65- 4.56 (m, 1H), 4.47-4.39 (m, 1H), 3.37- 3.32 (m, 2H), 3.25-3.19 (m, 1H), 3.18- 3.11 (m, 1H), 3.06 (s, 3H), 2.90 (t, J = 7.7 Hz, 2H), 2.74-2.61 (m, 2H), 2.47 (s, 3H), 2.45 (s, 3H), 2.30-2.19 (m, 2H). | U: 329 [M + H]⁺ |
| Example 365 | 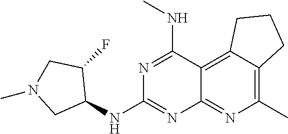 | Example 230, separating other regioisomer, then as per Example 233, using appropriate amine | ¹H NMR (400 MHz, Methanol-d₄) δ 5.17- 4.95 (m, 1H), 4.72 (dt, J = 25.8, 7.6 Hz, 1H), 3.40-3.31 (m, 3H), 3.07 (s, 3H), 3.04-2.92 (m, 1H), 2.94-2.74 (m, 3H), 2.45 (s, 3H), 2.40 (s, 3H), 2.38-2.33 (m, 1H), 2.31-2.18 (m, 2H). | AM: 331 [M + H]⁺ |
| Example 366 | 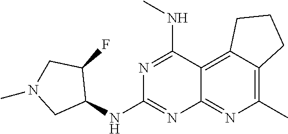 | Example 230, separating other regioisomer, then as per Example 232, using appropriate amine | ¹H NMR (400 MHz, DMSO-d₆) δ 6.59 (s, 1H), 6.44 (s, 1H), 5.28-5.04 (m, 1H), 4.59- 4.43 (m, 1H), 3.33-3.27 (m, 2H), 3.25-3.09 (m, 1H), 2.95 (d, J = 4.4 Hz, 3H), 2.89 (t, J = 8.1 Hz, 1H), 2.82 (t, J = 7.7 Hz, 2H), 2.66-2.52 (m, 2H), 2.38 (s, 3H), 2.30 (s, 3H), 2.11 (p, J = 7.6 Hz, 2H). | U: 331 [M + H]⁺ |
| Example 367 | 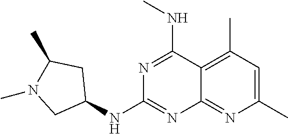 | Example 227, with appropriate amine, followed by reductive amination per Example 233 | ¹H NMR (400 MHz, DMSO-d₆) δ 6.88-6.63 (m, 2H), 6.54 (s, 1H), 4.33-4.20 (m, 1H), 2.99-2.88 (m, 4H), 2.65 (s, 3H), 2.43-2.29 (m, 5H), 2.19 (s, 3H), 2.17-2.08 (m, 1H), 1.51-1.38 (m, 1H), 1.08 (d, J = 6.0 Hz, 3H). | S: 301 [M + H]⁺ |
| Example 368 | 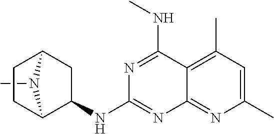 | Example 227, with appropriate amine, followed by reductive amination per Example 228 | ¹H NMR (400 MHz, DMSO-d₆) δ 6.99-6.57 (m, 3H), 4.14 (s, 1H), 3.51-3.43 (m, 1H), 3.07 (t, J = 4.6 Hz, 1H), 2.95 (d, J = 4.3 Hz, 3H), 2.64 (s, 3H), 2.37 (s, 3H), 2.20 (s, 3H), 2.10-1.96 (m, 1H), 1.79-1.59 (m, 2H), 1.56-1.37 (m, 2H), 1.27- 1.10 (m, 1H). | S: 313 [M + H]⁺ |
| | Mixture of trans isomers | | | |
| Example 369 | 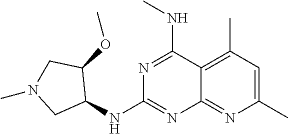 | Example 227, with appropriate amine, followed by reductive amination per Example 228 | ¹H NMR (400 MHz, DMSO-d₆) δ 6.80- 6.66 (m, 2H), 5.93 (s, 1H), 4.50 (t, J = 7.3 Hz, 1H), 3.94-3.87 (m, 1H), 3.22 (s, 3H), 3.03-2.83 (m, 5H), 2.65 (s, 3H), 2.46-2.31 (m, 5H), 2.26 (s, 3H). | S: 317 [M + H]⁺ |
| Example 370 | 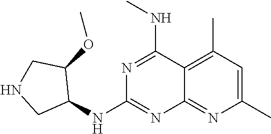 [SMILES: CNc1nc(N[C@H]2CNC[C@H]2OC)nc2nc(C)cc(C)c12] | Example 227, with appropriate amine | ¹H NMR (400 MHz, Methanol-d₄) δ 6.80 (s, 1H), 4.63-4.55 (m, 1H), 4.01-3.94 (m, 1H), 3.40 (s, 3H), 3.39-3.33 (m, 1H), 3.18-3.11 (m, 2H), 3.07 (s, 3H), 2.84 (dd, J = 11.1, 8.4 Hz, 1H), 2.71 (s, 3H), 2.47 (s, 3H). | S: 303 [M + H]⁺ |
| Example 371 | 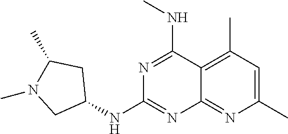 | Example 227, with appropriate amine, followed by reductive amination per Example 228 | ¹H NMR (400 MHz, Methanol-d₄) δ 6.77 (s, 1H), 4.59-4.48 (m, 1H), 3.12-2.98 (m, 4H), 2.70 (s, 3H), 2.66-2.60 (m, 1H), 2.59-2.51 (m, 1H), 2.45 (s, 3H), 2.32 (s, 4H), 1.49- 1.39 (m, 1H), 1.19 (d, J = 6.1 Hz, 3H). | U: 301 [M + H]⁺ |
| Example 372 | 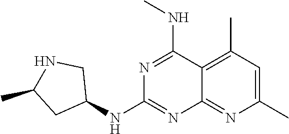 | Example 227, with appropriate amine | ¹H NMR (400 MHz, Methanol-d₄) δ 6.79 (s, 1H), 4.60-4.50 (m, 1H), 3.29-3.20 (m, 2H), 3.15-3.00 (m, 4H), 2.71 (s, 3H), 2.53-2.41 (m, 4H), 1.56-1.39 (m, 1H), 1.32 (d, J = 6.4 Hz, 3H). | AA: 287 [M + H]⁺ |
| Example 373 | 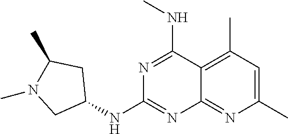 | Example 227, with appropriate amine, followed by reductive amination per Example 233 | ¹H NMR (400 MHz, Methanol-d₄) δ 6.79 (s, 1H), 4.68-4.56 (m, 1H), 3.63 (t, J = 8.7 Hz, 1H), 3.07 (s, 3H), 2.78-2.63 (m, 4H), 2.46 (s, 3H), 2.44-2.32 (m, 4H), 2.06-1.96 (m, 2H), 1.19 (d, J = 6.2 Hz, 3H). | T: 301 [M + H]⁺ |
| Example 374 | 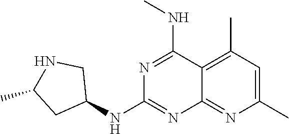 | Example 227, with appropriate amine | ¹H NMR (400 MHz, Methanol-d₄) δ 6.78 (s, 1H), 4.65-4.55 (m, 1H), 3.55-3.41 (m, 2H), 3.05 (s, 3H), 2.90 (dd, J = 12.3, 4.8 Hz, 1H), 2.71 (s, 3H), 2.46 (s, 3H), 2.08-1.98 (m, 1H), 1.85-1.72 (m, 1H), 1.25 (d, J = 6.5 Hz, 3H). | T: 287 [M + H]⁺ |
| Example 375 | 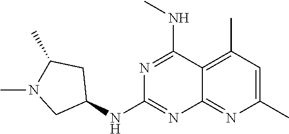 | Example 227, with appropriate amine, followed by reductive amination per Example 233 | ¹H NMR (400 MHz, Methanol-d₄) δ 6.76 (s, 1H), 4.66-4.53 (m, 1H), 3.63-3.55 (m, 1H), 3.06 (s, 3H), 2.70 (d, J = 0.8 Hz, 3H), 2.60- 2.49 (m, 1H), 2.45 (s, 3H), 2.35 (s, 3H), 2.27- 2.20 (m, 1H), 2.01-1.91 (m, 2H), 1.15 (d, J = 6.2 Hz, 3H). | AA: 301 [M + H]⁺ |
| Example 376 | 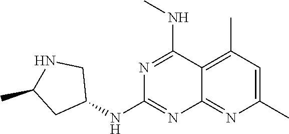 | Example 227, with appropriate amine | ¹H NMR (400 MHz, DMSO-d₆) δ 6.89- 6.52 (m, 3H), 4.47-4.33 (m, 1H), 3.30- 3.14 (m, 2H), 2.94 (d, J = 4.3 Hz, 3H), 2.72- 2.57 (m, 4H), 2.37 (s, 3H), 1.90-1.77 (m, 1H), 1.58-1.45 (m, 1H), 1.07 (d, J = 6.3 Hz, 3H). | AA: 287 [M + H]⁺ |
| Example 377 | 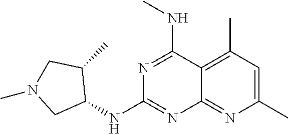 | Example 227, with appropriate amine, followed by reductive amination per Example 233 | ¹H NMR (400 MHz, DMSO-d₆) δ 6.91- 6.39 (m, 3H), 4.71-4.53 (m, 1H), 3.02-2.79 (m, 5H), 2.64 (s, 3H), 2.47-2.34 (m, 4H), 2.32-2.25 (m, 1H), 2.23 (s, 3H), 2.05-1.91 (m, 1H), 0.80 (d, J = 7.1 Hz, 3H). | AA: 301 [M + H]⁺ |
| Example 378 | 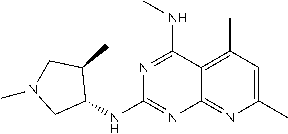 | Example 227, with appropriate amine, followed by reductive amination per Example 233 | ¹H NMR (400 MHz, Methanol-d₄) δ 6.76 (s, 1H), 4.30-4.21 (m, 1H), 3.11-2.88 (m, 5H), 2.79-2.61 (m, 4H), 2.45 (s, 3H), 2.37 (s, 3H), 2.27-2.12 (m, 2H), 1.19 (d, J = 6.6 Hz, 3H). | W: 301 [M + H]⁺ |
| Example 379 | 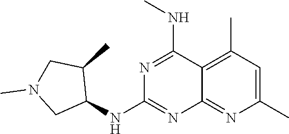 | Example 227, with appropriate amine, followed by reductive amination per Example 233 | ¹H NMR (400 MHz, DMSO-d₆) δ 6.86-6.40 (m, 3H), 4.67-4.53 (m, 1H), 3.03-2.89 (m, 4H), 2.88-2.80 (m, 1H), 2.64 (s, 3H), 2.45- 2.34 (m, 4H), 2.32-2.24 (m, 1H), 2.23 (s, 3H), 2.03-1.91 (m, 1H), 0.80 (d, J = 7.1 Hz, 3H). | AA: 301 [M + H]⁺ |
| Example 380 | 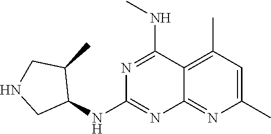 [SMILES: CNc1nc(N[C@H]2CNC[C@H]2C)nc2nc(C)cc(C)c12] | Example 227, with appropriate amine | ¹H NMR (400 MHz, Methanol-d₄) δ 6.77 (s, 1H), 4.73-4.59 (m, 1H), 3.39-3.33 (m, 1H), 3.26-3.19 (m, 1H), 3.12-2.96 (m, 4H), 2.80- 2.67 (m, 4H), 2.53-2.42 (m, 4H), 1.02 (d, J = 7.0 Hz, 3H). | AA: 287 [M + H]⁺ |
| Example 381 | 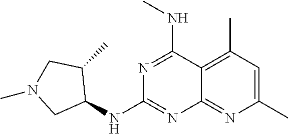 | Example 227, with appropriate amine, followed by reductive amination per Example 233 | ¹H NMR (400 MHz, DMSO-d₆) δ 6.93- 6.58 (m, 3H), 4.11-3.93 (m, 1H), 3.05-2.88 (m, 3H), 2.75 (t, J = 8.1 Hz, 2H), 2.65 (s, 3H), 2.44-2.32 (m, 4H), 2.26-2.09 (m, 4H), 2.09-2.01 (m, 1H), 1.07 (d, J = 6.8 Hz, 3H). | AA: 301 [M + H]⁺ |
| Example 382 | 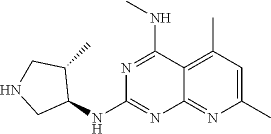 [SMILES: CNc1nc(N[C@H]2CNC[C@@H]2C)nc2nc(C)cc(C)c12] | Example 227, with appropriate amine | ¹H NMR (400 MHz, DMSO-d₆) δ 7.01- 6.56 (m, 3H), 4.04-3.90 (m, 1H), 3.17- 3.01 (m, 2H), 2.94 (d, J = 4.3 Hz, 3H), 2.70-2.55 (m, 4H), 2.40-2.29 (m, 4H), 2.11-1.95 (m, 1H), 1.02 (d, J = 6.8 Hz, 3H). | AB: 287 [M + H]⁺ |
| Example 383 | 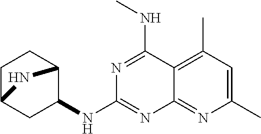 | Example 227, with appropriate amine | ¹H NMR (400 MHz, DMSO-d₆) δ 6.93- 6.59 (m, 2H), 6.22-5.86 (m, 1H), 3.83-3.74 (m, 1H), 3.14 (t, J = 4.3 Hz, 1H), 3.10 (d, J = 3.9 Hz, 1H), 2.92 (s, 3H), 2.64 (s, 3H), 2.37 (s, 3H), 1.81-1.71 (m, 1H), 1.70-1.50 (m, 3H), 1.34-1.14 (m, 2H). | S: 299 [M + H]⁺ |
| | Mixture of cis isomers | | | |
| Example 384 | 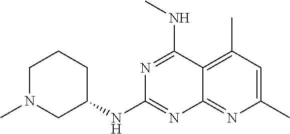 | Example 142, with appropriate amines | ¹H NMR (400 MHz, DMSO-d₆) δ 6.86- 6.60 (m, 2H), 6.39 (s, 1H), 4.07-3.94 (m, 1H), 2.93 (d, J = 4.3 Hz, 3H), 2.86-2.72 (m, 1H), 2.64 (s, 3H), 2.56 (d, J = 10.8 Hz, 1H), 2.37 (s, 3H), 2.16 (s, 3H), 1.94-1.70 (m, 3H), 1.68-1.59 (m, 1H), 1.56-1.46 (m, 1H), 1.38-1.23 (m, 1H). | S: 301 [M + H]⁺ |
| Example 385 | 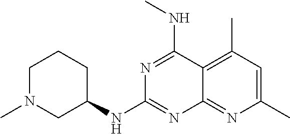 | Example 142, with appropriate amines | ¹H NMR (400 MHz, DMSO-d₆) δ 6.87- 6.60 (m, 2H), 6.52-6.33 (m, 1H), 4.07-3.93 (m, 1H), 2.93 (d, J = 4.2 Hz, 3H), 2.85- 2.74 (m, 1H), 2.64 (s, 3H), 2.61-2.53 (m, 1H), 2.37 (s, 3H), 2.17 (s, 3H), 1.98-1.60 (m, 4H), 1.58-1.44 (m, 1H), 1.37-1.21 (m, 1H). | S: 301 [M + H]⁺ |
| Example 386 | 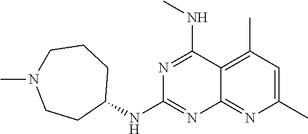 | Example 228, using appropriate amine | ¹H NMR (400 MHz, Methanol-d₄) δ 6.74 (s, 1H), 4.34-4.23 (m, 1H), 3.05 (s, 3H), 2.90- 2.80 (m, 1H), 2.79-2.71 (m, 2H), 2.70-2.63 (m, 4H), 2.45 (s, 3H), 2.41 (s, 3H), 2.19- 2.06 (m, 2H), 1.92-1.79 (m, 2H), 1.77-1.64 (m, 2H). | AA: 315 [M + H]⁺ |
| Example 387 | 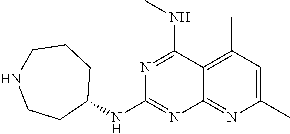 [SMILES: CNc1nc(N[C@H]2CCCNCC2)nc2nc(C)cc(C)c12] | Example 227, with appropriate amine | ¹H NMR (400 MHz, Methanol-d₄) δ 6.76 (s, 1H), 4.28-4.17 (m, 1H), 3.16-2.90 (m, 7H), 2.70 (s, 3H), 2.46 (s, 3H), 2.24-2.16 (m, 1H), 2.16-2.08 (m, 1H), 1.91-1.67 (m, 4H). | AA: 301 [M + H]⁺ |
| Example 388 | 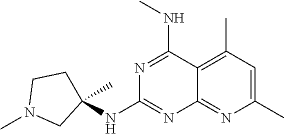 | Example 227, with appropriate amine, followed by reductive amination per Example 233 | ¹H NMR (400 MHz, DMSO-d₆) δ 6.92- 6.34 (m, 3H), 2.95 (d, J = 4.2 Hz, 3H), 2.82 (d, J = 9.9 Hz, 1H), 2.74 (d, J = 9.5 Hz, 1H), 2.65 (s, 3H), 2.61-2.54 (m, 2H), 2.38 (s, 3H), 2.30-2.17 (m, 4H), 1.90-1.78 (m, 1H), 1.54 (s, 3H). | W: 301 [M + H]⁺ |
| Example 389 | 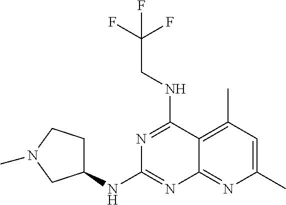 | Example 5 (Route B), with appropriate amines | ¹H NMR (400 MHz, DMSO-d₆) δ 7.36- 6.87 (m, 2H), 6.76 (s, 1H), 4.49-4.31 (m, 3H), 2.74 (t, J = 8.2 Hz, 1H), 2.68 (s, 3H), 2.59-2.52 (m, 1H), 2.46-2.31 (m, 5H), 2.24 (s, 3H), 2.20-2.05 (m, 1H), 1.75-1.63 (m, 1H). | S: 355 [M + H]⁺ |
| Example 390 | 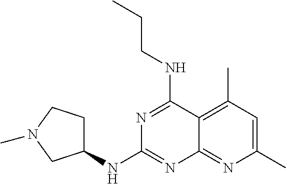 | Example 5 (Route B), with appropriate amines | ¹H NMR (400 MHz, DMSO-d₆) δ 6.90- 6.52 (m, 3H), 4.46-4.30 (m, 1H), 3.49- 3.38 (m, 2H), 2.79-2.70 (m, 1H), 2.66 (s, 3H), 2.57-2.52 (m, 1H), 2.43 (t, J = 7.5 Hz, 1H), 2.39-2.31 (m, 4H), 2.24 (s, 3H), 2.19- 2.07 (m, 1H), 1.76-1.58 (m, 3H), 0.93 (t, J = 7.4 Hz, 3H). | T: 315 [M + H]⁺ |
| Example 391 | 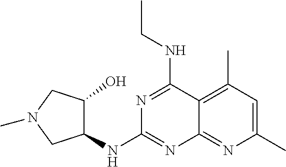 | Example 228, using appropriates amines | ¹H NMR (400 MHz, DMSO-d₆) δ 6.99 (s, 1H), 6.74 (s, 2H), 6.07 (s, 1H), 3.95 (d, J = 26.9 Hz, 2H), 3.51 (p, J = 6.7 Hz, 2H), 2.86 (t, J = 8.4 Hz, 1H), 2.77-2.64 (m, 4H), 2.49- 2.33 (m, 5H), 2.21 (s, 3H), 1.21 (t, J = 7.1 Hz, 3H). | S: 317 [M + H]⁺ |
| Example 392 | 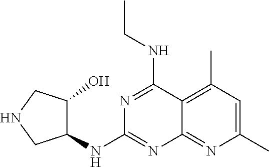 [SMILES: CCNc1nc(N[C@H]2CNC[C@@H]2O)nc2nc(C)cc(C)c12] | Example 227, using appropriate amines | ¹H NMR (400 MHz, Methanol-d₄) δ 6.81 (s, 1H), 4.31-4.26 (m, 1H), 4.23 (s, 1H), 3.61 (q, J = 7.1 Hz, 2H), 3.46 (dd, J = 12.2, 6.8 Hz, 1H), 3.24 (dd, J = 12.3, 5.3 Hz, 1H), 2.99 (dd, J = 12.2, 3.9 Hz, 1H), 2.91 (dd, J = 12.3, 2.9 Hz, 1H), 2.73 (s, 3H), 2.48 (s, 3H), 1.28 (t, J = 7.2 Hz, 3H). | AA: 203 [M + H]⁺ |
| Example 393 | 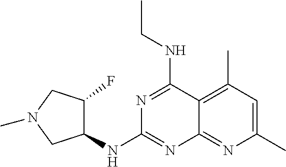 | Example 228, using appropriate amines | ¹H NMR (400 MHz, Methanol-d₄) δ 6.78 (s, 1H), 5.03 (dd, J = 52.9, 4.9 Hz, 1H), 4.71 (dt, J = 24.7, 6.9 Hz, 1H), 3.64 (q, J = 7.1 Hz, 2H), 3.40-3.33 (m, 1H), 3.07-2.91 (m, 1H), 2.85-2.74 (m, 1H), 2.72 (s, 3H), 2.46 (s, 3H), 2.39 (s, 3H), 2.37-2.28 (m, 1H), 1.29 (t, J = 7.1 Hz, 3H). | AK: 319 [M + H]⁺ |
| Example 394 | 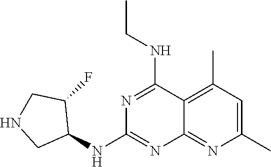 [SMILES: CCNc1nc(N[C@H]2CNC[C@@H]2F)nc2nc(C)cc(C)c12] | Example 227, using appropriate amines | ¹H NMR (400 MHz, Methanol-d₄) δ 6.80 (s, 1H), 5.13 (dd, J = 52.2, 3.8 Hz, 1H), 4.65- 4.53 (m, 1H), 3.71-3.57 (m, 2H), 3.42 (dd, J = 12.3, 6.6 Hz, 1H), 3.25-3.03 (m, 2H), 2.89 (d, J = 12.1 Hz, 1H), 2.73 (s, 3H), 2.47 (s, 3H), 1.29 (t, J = 7.1 Hz, 3H). | AK: 305 [M + H]⁺ |
| Example 395 | 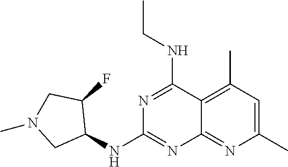 | Example 232, with appropriate amines, followed by reductive amination per Example 233 | ¹H NMR (400 MHz, DMSO-d₆) δ 6.96-6.64 (m, 2H), 6.54 (s, 1H), 5.13 (d, J = 55.7 Hz, 1H), 4.50 (dp, J = 22.1, 7.8 Hz, 1H), 3.51 (p, J = 6.7 Hz, 2H), 3.23-3.11 (m, 1H), 2.88 (t, J = 8.1 Hz, 1H), 2.67 (s, 3H), 2.65- 2.52 (m, 2H), 2.38 (s, 3H), 2.30 (s, 3H), 1.21 (t, J = 7.1 Hz, 3H). | AA: 319 [M + H]⁺ |
| Example 396 | 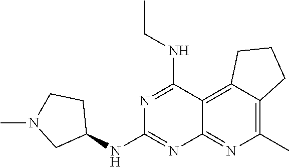 | Example 230, separating other regioisomer, then as per Example 234 using appropriate amines | ¹H NMR (400 MHz, Methanol-d₄) δ 4.69- 4.60 (m, 1H), 3.59 (q, J = 7.2 Hz, 2H), 3.38- 3.33 (m, 2H), 2.98 (s, 1H), 2.90 (t, J = 7.6 Hz, 2H), 2.76 (s, 1H), 2.67-2.51 (m, 2H), 2.45 (s, 3H), 2.39 (s, 4H), 2.30-2.17 (m, 2H), (m, 2H), 1.84-1.74 (m, 1H), 1.28 (t, J = 7.1 Hz, 3H). | W: 327 [M + H]⁺ |
| Example 397 | 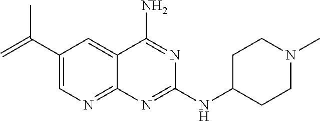 [SMILES: C=C(C)c1cnc2nc(NC3CCN(C)CC3)nc(N)c2c1] | Example 238, using Example 20 | ¹H NMR (400 MHz, DMSO-d₆) δ 8.83 (s, 1H), 8.44 (s, 1H), 7.49 (br, 2H), 6.75 (br, 1H), 5.53 (s, 1H), 5.10 (s, 1H), 3.91-3.67 (m, 1H), 2.75 (d, J = 11.4 Hz, 2H), 2.16 (s, 3H), 2.15 (s, 3H), 1.94 (t, J = 11.7 Hz, 2H), 1.81 (d, J = 12.2 Hz, 2H), 1.64-1.43 (m, 2H). | AJ: 299 [M + H]⁺ |
| Example 398 | 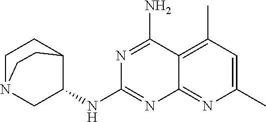 [SMILES: Cc1cc(C)c2c(N)nc(N[C@@H]3CN4CCC3CC4)nc2n1] | Example 142, using appropriate amine | ¹H NMR (400 MHz, Methanol-d₄) δ 6.79 (s, 1H), 4.48-4.29 (m, 2H), 3.71 (dd, J = 14.1, 4.1 Hz, 1H), 3.27-3.08 (m, 2H), 3.03-2.92 (m, 1H), 2.91-2.75 (m, 1H), 2.71 (s, 3H), 2.47 (s, 3H), 2.37-2.28 (m, 1H), 2.08-1.95 (m, 1H), 1.83-1.60 (m, 2H), 1.58-1.42 (m, 1H). | W: 299 [M + H]⁺ |
| Example 399 | 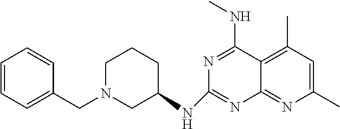 [SMILES: CNc1nc(N[C@@H]2CCCN(Cc3ccccc3)C2)nc2nc(C)cc(C)c12] | Example 228, using appropriate amine | ¹H NMR (400 MHz, DMSO-d₆) δ 7.36- 7.27 (m, 4H), 7.26-7.20 (m, 1H), 6.82-6.58 (m, 2H), 6.43 (d, J = 8.6 Hz, 1H), 4.15-3.91 (m, 1H), 3.64-3.36 (m, 2H), 3.00-2.78 (m, 4H), 2.69-2.56 (m, 4H), 2.36 (s, 3H), 2.00- 1.74 (m, 3H), 1.71-1.59 (m, 1H), 1.58-1.43 (m, 1H), 1.39-1.25 (m, 1H). | AN: 377 [M + H]⁺ |
| Example 400 | 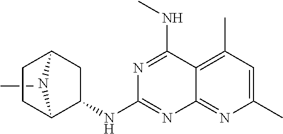 | Example 228, using appropriate amine | ¹H NMR (400 MHz, DMSO-d₆) δ 6.87-6.61 (m, 2H), 6.27-5.87 (m, 1H), 3.85-3.74 (m, 1H), 3.14 (t, J = 4.3 Hz, 1H), 3.10 (d, J = 3.9 Hz, 1H), 2.92 (s, 3H), 2.64 (s, 3H), 2.37 (s, 3H), 2.14 (s, 3H), 1.81-1.62 (m, 3H), 1.60-1.49 (m, 1H), 1.34-1.17 (m, 2H). | AO: 313 [M + H]⁺ |
| | Mixture of cis isomers | | | |
| Example 401 | 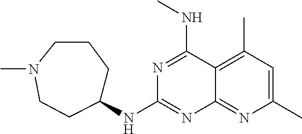 | Example 228, using appropriate amine | ¹H NMR (400 MHz, DMSO-d₆) δ 6.84-6.62 (m, 2H), 6.52 (s, 1H), 4.19-4.07 (m, 1H), 2.93 (d, J = 4.3 Hz, 3H), 2.64 (s, 3H), 2.61- 2.53 (m, 2H), 2.48-2.39 (m, 2H), 2.36 (s, 3H), 2.25 (s, 3H), 1.96-1.81 (m, 2H), 1.78- 1.61 (m, 3H), 1.61-1.49 (m, 1H). | AK: 315 [M + H]⁺ |
| Example 402 | 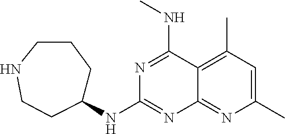 [SMILES: CNc1nc(N[C@@H]2CCCNCC2)nc2nc(C)cc(C)c12] | Example 227, using appropriate amine. Deprotection as per Example 140 | ¹H NMR (400 MHz, DMSO-d₆) δ 6.89- 6.58 (m, 2H), 6.50 (s, 1H), 4.21-4.05 (m, 1H), 2.93 (d, J = 4.9 Hz, 3H), 2.88-2.79 (m, 1H), 2.79-2.66 (m, 3H), 2.64 (s, 3H), 2.36 (s, 3H), 1.98-1.87 (m, 1H), 1.86-1.77 (m, 1H), 1.73-1.59 (m, 3H), 1.58-1.43 (m, 1H). | AA: 301 [M + H]⁺ |
| Example 403 | 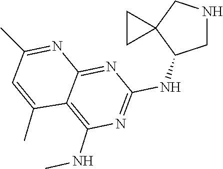 [SMILES: CNc1nc(N[C@H]2CNCC23CC3)nc2nc(C)cc(C)c12] | Example 219, using intermediate from Example 227 and appropriate amine. Deprotection as per Example 140 | | Library F: 299 [M + H]+ |
| Example 404 | 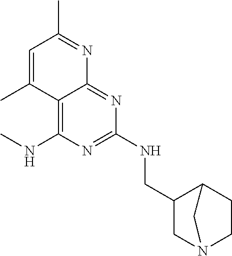 [SMILES: CNc1nc(NCC2CN3CCC2C3)nc2nc(C)cc(C)c12] | Example 219, using intermediate from Example 227 and appropriate amine | | Library C: 313 [M + H]+ |
| Example 405 | 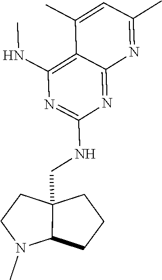 | Example 219, using intermediate from Example 227 and appropriate amine | | Library C: 341 [M + H]+ |
| | Mixture of trans isomers | | | |
| Example 406 | 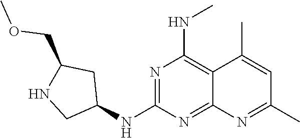 | Example 219, using intermediate from Example 227 and appropriate amine. Deprotection | | Library C: 317 [M + H]+ |
| | | as per | | |
| | | Example 140 | | |
| Example 407 | 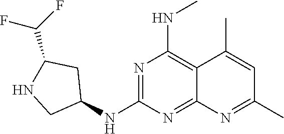 [SMILES: CNc1nc(N[C@H]2CN[C@H](C(F)F)C2)nc2nc(C)cc(C)c12] | Example 219, using intermediate from Example 227 and appropriate amine. Deprotection | | Library C: 323 [M + H]+ |
| | | as per | | |
| | | Example 140 | | |
| Example 408 | 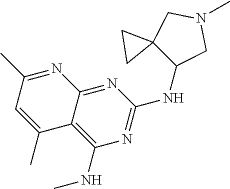 | Example 219, using intermediate from Example 227 and appropriate amine | | Library C: 313 [M + H]+ |
| Example 409 | 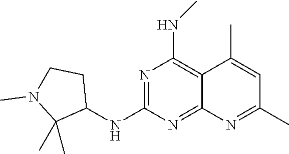 | Example 219, using intermediate from Example 227 and appropriate amine | | Library C: 315 [M + H]+ |
| Example 410 | 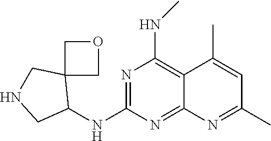 [SMILES: CNc1nc(NC2CNCC23COC3)nc2nc(C)cc(C)c12] | Example 219, using intermediate from Example 227 and appropriate amine. Deprotection | | Library E: 315 [M + H]+ |
| | | as per | | |
| | | Example 140 | | |
| Example 411 | 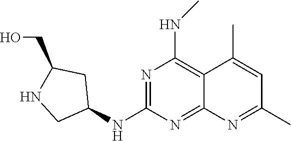 [SMILES: CNc1nc(N[C@H]2CN[C@@H](CO)C2)nc2nc(C)cc(C)c12] | Example 219, using intermediate from Example 227 and appropriate amine. Deprotection | | Library K: 303 [M + H]+ |
| | | as per | | |
| | | Example 140 | | |
| Example 412 | 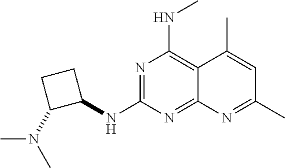 | Example 219, using intermediate from Example 227 and appropriate amine | | Library C: 301 [M + H]+ |
| | Mixture of trans isomers | | | |
| Example 413 | 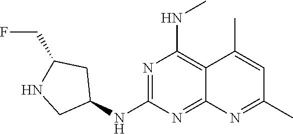 [SMILES: CNc1nc(N[C@H]2CN[C@H](CF)C2)nc2nc(C)cc(C)c12] | Example 219, using intermediate from Example 227 and appropriate amine. Deprotection | | Library C: 305 [M + H]+ |
| | | as per | | |
| | | Example 140 | | |
| Example 414 | 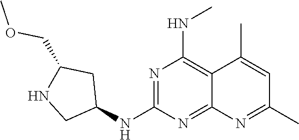 | Example 219, using intermediate from Example 227 and appropriate amine. Deprotection | | Library C: 317 [M + H]+ |
| | | as per | | |
| | | Example 140 | | |
| Example 415 | 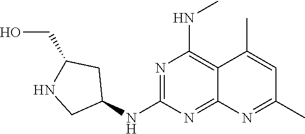 [SMILES: CNc1nc(N[C@H]2CN[C@H](CO)C2)nc2nc(C)cc(C)c12] | Example 219, using intermediate from Example 227 and appropriate amine. Deprotection | | Library C: 303 [M + H]+ |
| | | as per | | |
| | | Exmaple 140 | | |
| Example 416 | 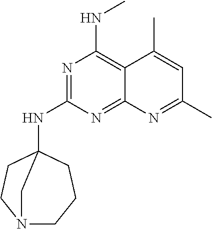 [SMILES: CNc1nc(NC23CCCN(CC2)C3)nc2nc(C)cc(C)c12] | Example 219, using intermediate from Example 227 and appropriate amine | | Library C: 313 [M + H]+ |
| Example 417 | 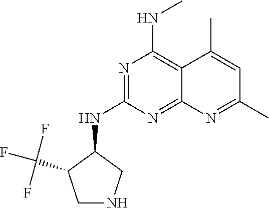 [SMILES: CNc1nc(N[C@H]2CNC[C@@H]2C(F)(F)F)nc2nc(C)cc(C)c12] | Example 219, using intermediate from Example 227 and appropriate amine. Deprotection as per Example 140 | | Library C: 341 [M + H]+ |
| Example 418 | 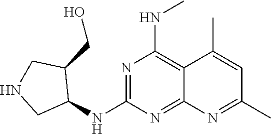 [SMILES: CNc1nc(N[C@H]2CNC[C@H]2CO)nc2nc(C)cc(C)c12] | Example 219, using intermediate from Example 227 and appropriate amine. Deprotection | | Library A: 303 [M + H]+ |
| | | as per | | |
| | | Example 140 | | |
| Example 419 | 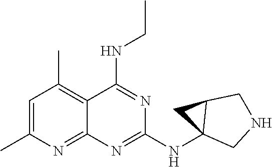 [SMILES: CCNc1nc(N[C@@]23CNC[C@@H]2C3)nc2nc(C)cc(C)c12] | Example 227, using appropriate amines | ¹H NMR (400 MHz, DMSO-d₆) δ 7.07 (s, 1H), 6.89-6.55 (m, 2H), 3.62-3.49 (m, 2H), 3.00 (d, J = 10.0 Hz, 2H), 2.89-2.79 (m, 1H), 2.73-2.60 (m, 4H), 2.73-2.60 (m, 4H), 2.37 (s, 3H), 1.51-1.36 (m, 1H), 1.51- 1.36 (m, 1H), 1.20 (t, J = 7.1 Hz, 3H), 0.88 (t, J = 4.6 Hz, 1H), 0.78-0.64 (m, 1H). | W: 299 [M + H]⁺ |
| Example 420 | 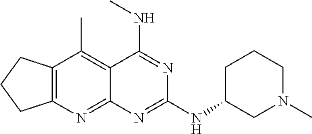 [SMILES: CNc1nc(N[C@@H]2CCCN(C)C2)nc2nc3c(c(C)c12)CCC3] | Example 231, using appropriate amine | ¹H NMR (400 MHz, DMSO-d₆) δ 6.72 (s, 1H), 6.29 (s, 1H), 4.06-3.90 (m, 1H), 2.93 (d, J = 4.3 Hz, 3H), 3.90-2.77 (m, 5H), 2.66- 2.53 (m, 4H), 2.16 (s, 3H), 2.02 (p, J = 7.6 Hz, 2H), 1.95-1.70 (m, 3H), 1.70-1.59 (m, 1H), 1.58-1.42 (m, 1H), 1.36-1.21 (m, 1H). | AD: 327 [M + H]⁺ |
| Example 421 | 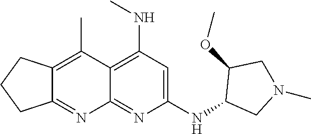 [SMILES: CNc1cc(N[C@H]2CN(C)C[C@@H]2OC)nc2nc3c(c(C)c12)CCC3] | Example 425, using appropriate amine | ¹H NMR (400 MHz, DMSO-d₆) δ 6.75 (d, J = 7.8 Hz, 1H), 5.83 (d, J = 5.1 Hz, 1H), 5.71 (s, 1H), 4.43-4.34 (m, 1H), 3.76-3.68 (m, 1H), 3.29 (s, 3H), 2.98-2.77 (m, 6H), 2.75 (d, J = 4.7 Hz, 3H), 2.64 (s, 3H), 2.42-2.36 (m, 1H), 2.31-2.26 (m, 1H), 2.22 (s, 3H), 2.07- 1.97 (m, 2H). | W: 342 [M + H]⁺ |
| Example 422 | 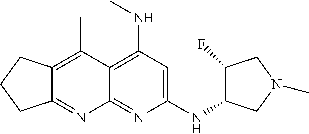 [SMILES: CNc1cc(N[C@H]2CN(C)C[C@H]2F)nc2nc3c(c(C)c12)CCC3] | Example 425, using appropriate amine | ¹H NMR (400 MHz, Methanol-d₄) δ 5.82 (s, 1H), 5.31-5.12 (m, 1H), 4.97-4.88 (m, 1H), 3.15-2.91 (m, 7H), 2.88 (s, 3H), 2.70 (s, 4H), 2.43 (s, 3H), 2.18-2.08 (m, 2H). | AB: 330 [M + H]⁺ |
| Example 423 | 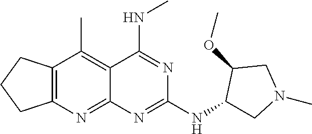 [SMILES: CNc1nc(N[C@H]2CN(C)C[C@@H]2OC)nc2nc3c(c(C)c12)CCC3] | Example 232, using appropriate amine | ¹H NMR (400 MHz, Methanol-d₄) δ 4.64- 4.56 (m, 1H), 3.89-3.82 (m, 1H), 3.40 (s, 3H), 3.20-3.11 (m, 1H), 3.07 (s, 3H), 3.02- 2.83 (m, 5H), 2.72-2.60 (m, 4H), 2.50-2.40 (m, 1H), 2.36 (s, 3H), 2.13 (p, J = 7.6 Hz, 2H). | AK: 343 [M + H]⁺ |
| Example 424 | 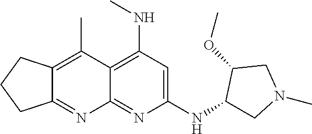 [SMILES: CNc1cc(N[C@H]2CN(C)C[C@H]2OC)nc2nc3c(c(C)c12)CCC3] | Example 425, using appropriate amine | ¹H NMR (400 MHz, Methanol-d₄) δ 5.80 (s, 1H), 4.81-4.72 (m, 1H), 4.07-3.97 (m, 1H), 3.34 (s, 3H), 3.06-2.92 (m, 5H), 2.89 (s, 3H), 2.88-2.85 (m, 1H), 2.77 (dd, J = 10.9, 3.2 Hz, 1H), 2.71 (s, 3H), 2.62 (dd, J = 9.8, 7.0 Hz, 1H), 2.38 (s, 3H), 2.13 (p, J = 7.7 Hz, 2H). | AA: 342 [M + H]+ |

### Example 425: (3S,4S)-1-methyl-4-((5-methyl-4-(methylamino)-7,8-dihydro-6H-cyclopenta[b][1,8]naphthyridin-2-yl)amino)pyrrolidin-3-ol

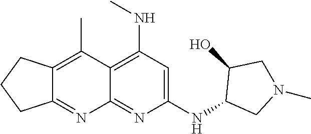 [SMILES: CNc1cc(N[C@H]2CN(C)C[C@@H]2O)nc2nc3c(c(C)c12)CCC3]

### Preparation 170—tert-butyl (3S,4S)-3-hydroxy-4-((5-methyl-4-(methylamino)-7,8-dihydro-6H-cyclopenta[b][1,8]naphthyridin-2-yl)amino)pyrrolidine-1-carboxylate

To a stirred mixture of 2-chloro-N,5-dimethyl-7,8-dihydro-6H-cyclopenta[b][1,8]naphthyridin-4-amine (200 mg, 0.807 mmol) and tert-butyl (3S,4S)-3-amino-4-hydroxypyrrolidine-1-carboxylate (196 mg, 0.968 mmol) in 1,4-dioxane (4.00 mL) were added Pd-PEPPSI-IPentCl 2-methylpyridine (o-picoline) (67.0 mg, 0.081 mmol) and Cs₂CO₃ (526 mg, 1.61 mmol). The resulting mixture was stirred under nitrogen atmosphere at 100° C. for 1 h. The mixture was allowed to cool down to room temperature. The resulting mixture was filtered, the filter cake was washed with MeOH (3×3.0 mL). The filtrate was concentrated under reduced pressure. The residue was purified by Prep-HPLC^{AV} to afford the title compound as a grey solid (280 mg, 83.9%). LCMS m/z 414 [M+H]⁺

### Preparation 171—(3S,4S)-4-((5-methyl-4-(methylamino)-7,8-dihydro-6H-cyclopenta[b][1,8]naphthyridin-2-yl)amino)pyrrolidin-3-ol

A mixture of tert-butyl (3S,4S)-3-hydroxy-4-((5-methyl-4-(methylamino)-7,8-dihydro-6H-cyclopenta[b][1,8]naphthyridin-2-yl)amino)pyrrolidine-1-carboxylate (170 mg, 0.410 mmol) and HCl in 1,4-dioxane (4.00 mL, 4.0 M) was stirred for 1 h at room temperature. The resulting mixture was concentrated under reduced pressure to afford the title compound (170 mg) as a brown solid, which was used in the next step directly without further purification. LCMS m/z=314 [M+H]⁺

### Preparation 172—(3S,4S)-1-methyl-4-((5-methyl-4-(methylamino)-7,8-dihydro-6H-cyclopenta[b][1,8]naphthyridin-2-yl)amino)pyrrolidin-3-ol

To a stirred mixture of (3S,4S)-4-((5-methyl-4-(methylamino)-7,8-dihydro-6H-cyclopenta[b][1,8]naphthyridin-2-yl)amino)pyrrolidin-3-ol (91.0 mg, 0.290 mmol) and Et₃N (118 mg, 1.16 mmol) in DCE (2.00 mL) was added paraformaldehyde (35.0 mg, 1.16 mmol). The mixture was stirred at 40° C. for 3 h. Then to the above mixture was added STAB (246 mg, 1.16 mmol) at room temperature. The resulting mixture was stirred at 40° C. for additional 1 h. The mixture was allowed to cool down to room temperature. The reaction was quenched by the addition of water/ice (2.0 mL) at 0° C. The resulting mixture was concentrated under reduced pressure. The residue was dissolved in DCM/MeOH (20.0 mL, 5/1). The resulting mixture was filtered, the filter cake was washed with DCM/MeOH (3×1.0 mL, 5/1). The filtrate was concentrated under reduced pressure. The residue was purified by Prep-HPLC^{W} to afford the title compound as an off-white solid (37.4 mg, 39.4%). ¹H NMR (400 MHz, DMSO-d₆) δ 6.99 (d, J=3.4 Hz, 1H), 6.72 (s, 1H), 5.95 (q, J=4.8 Hz, 1H), 5.72 (s, 1H), 3.94-3.83 (m, 2H), 2.94-2.78 (m, 5H), 2.78-2.71 (m, 4H), 2.65 (s, 3H), 2.50-2.36 (m, 2H), 2.23 (s, 3H), 2.13-1.97 (m, 2H). LCMS^{AA} m/z=328 [M+H]⁺

### Example 426: N²-(1-(2-methoxybenzyl)piperidin-4-yl)-5,7-dimethylpyrido[2,3-d]pyrimidine-2,4-diamine

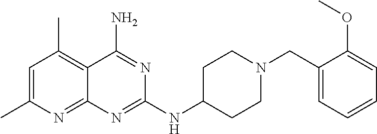 [SMILES: COc1ccccc1CN1CCC(Nc2nc(N)c3c(C)cc(C)nc3n2)CC1]

To a stirred mixture of 5,7-dimethyl-N²-(piperidin-4-yl)pyrido[2,3-d]pyrimidine-2,4-diamine (80 mg, 0.294 mmol, 1 equiv), 2-methoxybenzaldehyde (43.99 mg, 0.323 mmol, 1.1 equiv), acetic acid (0.059 mmol, 0.2 equiv) and sodium triacetoxyborohydride (124.51 mg, 0.588 mmol, 2 equiv) in DMAc (3 mL). The mixture was stirred at 0° C. for 10 min, then heated to 60° C. for 1 hour. The reaction was quenched with MeOH (0.5 mL) at 0° C. and the residue after evaporation was purified by Prep-HPLC to afford the desired product (15.6 mg). LCMS m/z Library^{C}=393 [M+H]⁺.

### Example 427: 2-((4-((4-amino-5,7-dimethylpyrido[2,3-d]pyrimidin-2-yl)amino)piperidin-1-yl)methyl)phenol

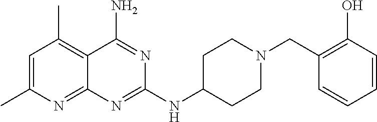 [SMILES: Cc1cc(C)c2c(N)nc(NC3CCN(Cc4ccccc4O)CC3)nc2n1]

### Preparation 173—5,7-dimethylpyrido[2,3-d]pyrimidine-2,4-diol

To a stirred mixture of 6-aminouracil (50.0 g, 393 mmol) in acetylacetone (200 mL) at room temperature was added PPA (90.5 g, 786 mmol). The resulting mixture was stirred at 100° C. for 1 h. The mixture was allowed to cool down to room temperature. The reaction was quenched with NaHCO₃(aq) at 0° C. The precipitated solids were collected by filtration and washed with water (2×300 mL). The solid was further purified by trituration with MtBE (300 mL). This resulted in title compound as an off-white solid (38.0 g, crude). LCMS m/z=192 [M+H]⁺

### Preparation 174—2,4-dichloro-5,7-dimethylpyrido[2,3-d]pyrimidine

To a stirred mixture of 5,7-dimethylpyrido[2,3-d]pyrimidine-2,4-diol (37.5 g, 196 mmol) in POCl₃ (375 mL) at 0° C. was added N,N-diethylaniline (146 g, 980 mmol) dropwise. The resulting mixture was stirred at 60° C. for 3 h. The resulting mixture was allowed to cool down to room temperature and concentrated under reduced pressure. The mixture basified to pH 8 with saturated NaHCO₃ (aq.). The resulting mixture was extracted with EtOAc (3×500 mL). The combined organic layers were washed with brine (500 mL), dried over anhydrous Na₂SO₄. After filtration, the filtrate was concentrated under reduced pressure. The residue was purified by silica gel column chromatography, eluted with PE/EA (2:1) to afford the title compound as a yellow solid (20.9 g, 46.9%). LCMS: m/z=228 [M+H]⁺

### Preparation 175—2-chloro-5,7-dimethylpyrido[2,3-d]pyrimidin-4-amine

A mixture of 2,4-dichloro-5,7-dimethylpyrido[2,3-d]pyrimidine (77.5 g, 341 mmol) in ammonium hydroxide (1.00 L) was stirred at room temperature for 3 h. The precipitated solids were collected by filtration and washed with water (3×200 mL). The solid was dried under reduced pressure to afford the title compound (65.9 g, crude) as an orange solid. LCMS m/z=209 [M+H]⁺.

### Preparation 176—2-tert-butyl 4-((4-amino-5,7-dimethylpyrido[2,3-d]pyrimidin-2-yl)amino)piperidine-1-carboxylate

A mixture of 2-chloro-5,7-dimethylpyrido[2,3-d]pyrimidin-4-amine (65.9 g, 317 mmol) and tert-butyl 4-aminopiperidine-1-carboxylate (127 g, 634 mmol) in DMSO (500 mL) was stirred at 100° C. overnight. The mixture was allowed to cool down to room temperature. The resulting mixture was diluted with water (1.50 L) and extracted with DCM (3×500 mL). The combined organic layers were washed with brine (3×500 mL), dried over anhydrous Na₂SO₄. After filtration, the filtrate was concentrated under reduced pressure. The residue was purified by trituration with MTBE (2×500 mL) to afford the title compound (106 g, 92.9%) as a pink solid. LCMS m/z=373 [M+H]⁺

### Preparation 177—5,7-dimethyl-N²-(piperidin-4-yl)pyrido[2,3-d]pyrimidine-2,4-diamine

A mixture of tert-butyl 4-((4-amino-5,7-dimethylpyrido[2,3-d]pyrimidin-2-yl)amino)piperidine-1-carboxylate (17.5 g, 47.0 mmol, 1.00 equiv) in 4 M HCl in 1,4-dioxane) (250 mL) was stirred for 1 h at room temperature. The resulting mixture was concentrated under reduced pressure. The residue was purified by trituration with EtOAc (200 mL). This resulted in the title compound as an off-white solid (12.0 g, crude). LCMS m/z=273 [M+H]⁺

### Preparation 178—2-((4-((4-amino-5,7-dimethylpyrido[2,3-d]pyrimidin-2-yl)amino)piperidin-1-yl)methyl)phenol

A mixture of 5,7-dimethyl-N²-(piperidin-4-yl)pyrido[2,3-d]pyrimidine-2,4-diamine (600 mg, 2.20 mmol) and 2-hydroxybenzaldehyde (538 mg, 4.40 mmol) in 1,2-dichloroethane (12.0 mL) was stirred at 40° C. for 3 h. To the above mixture was added STAB (1.86 g, 8.81 mmol) at room temperature. The resulting mixture was stirred at 40° C. for additional 0.5 h. The mixture was allowed to cool down to room temperature. The reaction was quenched with Water/Ice at 0° C. The resulting mixture was filtered, the filter cake was washed with MeOH (5×10 mL). The filtrate was concentrated under reduced pressure. The residue was purified by Prep-HPLC^{AW} to afford the title compound as a white solid (382.3 mg, 46.0%). ¹H NMR (400 MHz, DMSO-d₆) δ 10.90 (s, 1H), 7.14-7.04 (m, 2H), 6.88-6.38 (m, 6H), 3.96-3.83 (m, 1H), 3.66 (s, 2H), 2.90 (d, J=11.5 Hz, 2H), 2.63 (s, 3H), 2.37 (s, 3H), 2.17 (t, J=11.4 Hz, 2H), 1.89 (d, J=9.5 Hz, 2H), 1.61-1.46 (m, 2H). LCMS^{AJ} m/z=379 [M+H]⁺.

### Example 428: 4-((4-((4-amino-5,7-dimethylpyrido[2,3-d]pyrimidin-2-yl)amino)piperidin-1-yl)methyl)benzonitrile

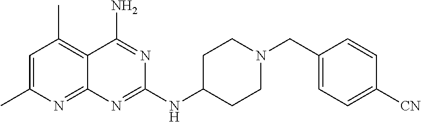 [SMILES: Cc1cc(C)c2c(N)nc(NC3CCN(Cc4ccc(C#N)cc4)CC3)nc2n1]

To a mixture of 5,7-dimethyl-N²-(piperidin-4-yl)pyrido[2,3-d]pyrimidine-2,4-diamine (500 mg, 1.84 mmol) in 1,2-dichloroethane (10 mL) was added 4-formylbenzonitrile (361 mg, 2.75 mmol) at room temperature. The resulting mixture was stirred at 40° C. for 3 h. To the above mixture was added STAB (778 mg, 3.67 mmol) at 40° C. The resulting mixture was stirred at 40° C. for additional 3 h. The mixture was allowed to cool down to room temperature. The reaction was quenched by the addition of Water (10 mL) at room temperature. The resulting mixture was concentrated under reduced pressure. The residue was purified by Prep-HPLC^{AV} to afford the title compound as an off-white solid (252.4 mg, 35.5%). ¹H NMR (400 MHz, DMSO-d₆) δ 7.84-7.76 (m, 2H), 7.52 (d, J=8.2 Hz, 2H), 6.99-6.54 (m, 3H), 6.38 (s, 1H), 3.92-3.75 (m, 1H), 3.56 (s, 2H), 2.78 (d, J=11.1 Hz, 2H), 2.63 (s, 3H), 2.37 (s, 3H), 2.14-2.00 (m, 2H), 1.82 (d, J=11.8 Hz, 2H), 1.59-1.45 (m, 2H). LCMS^{W} m/z=388 [M+H]⁺.

### Example 429-Example 506

These Examples were synthesised according to methods similar to those cited in the following table:

| | | Method | | LCMS |
|---|---|---|---|---|
| Example | Structure | Ref | ¹H NMR | m/z |
| Example 429 | 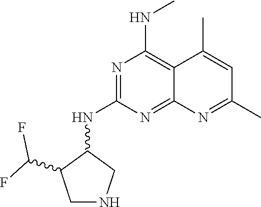 [SMILES: CNc1nc(NC2CNCC2C(F)F)nc2nc(C)cc(C)c12] | Example 219, using intermediate from Example 227 and appropriate amine. Deprotection as per Example 140 Purified by SFC^{Library B} R_{T} = 7.27 | | Library C: 323 [M + H]+ |
| | Isomer 1 - The stereochemistry | min. | | |
| | has not been unambiguously | | | |
| | confirmed | | | |
| Example 430 | 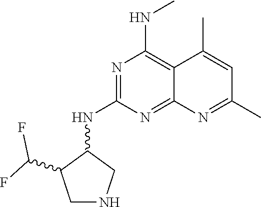 [SMILES: CNc1nc(NC2CNCC2C(F)F)nc2nc(C)cc(C)c12] | Example 219, using intermediate from Example 227 and appropriate amine. Deprotection as per Example 140 Purified by SFC^{Library B} R_{T} = 5.45 | | Library C: 323 [M + H]+ |
| | Isomer 2 - The stereochemistry | min. | | |
| | has not been unambiguously | | | |
| | confirmed | | | |
| Example 431 | 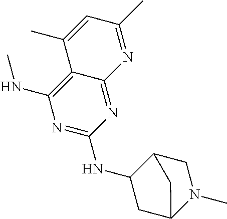 | Example 219, using intermediate from Example 227 and appropriate amine. Purified by SFC^{Library B} R_{T} = 6.97 min. | | Library C: 313 [M + H]+ |
| | Isomer 1 - The stereochemistry | | | |
| | has not been unambiguously | | | |
| | confirmed | | | |
| Example 432 | 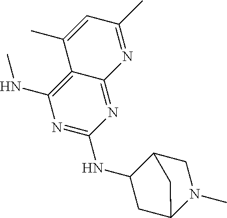 | Example 219, using intermediate from Example 227 and appropriate amine. SFC^{Library B} R_{T} = 6.04 min. | | Library C: 313 [M + H]+ |
| | Isomer 2 - The stereochemistry | | | |
| | has not been unambiguously | | | |
| | confirmed | | | |
| Example 433 | 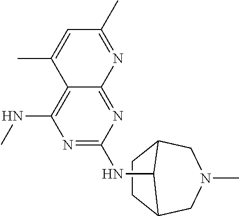 | Example 219, using intermediate from Example 227 and appropriate amine. Purified by SFC^{Library A} R_{T} = 10.80 min. | | Library C: 327 [M + H]⁺ |
| | Isomer 1 - cis or trans | | | |
| Example 434 | 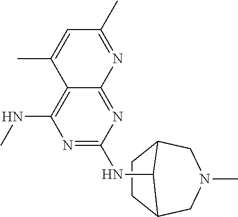 | Example 219, using intermediate from Example 227 and appropriate amine. Purified by SFC^{Library A} R_{T} = 8.10 min. | | Library C: 327 [M + H]+ |
| | Isomer 2 - cis or trans | | | |
| Example 435 | 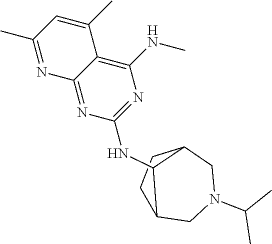 [SMILES: CNc1nc(NC2C3CCC2CN(C(C)C)C3)nc2nc(C)cc(C)c12] | Example 219, using intermediate from Example 227 and appropriate amine. Purified by HPLC^{Library} R_{T} = 2.74 min. | | Library C: 355 [M + H]+ |
| | Isomer 1 - cis or trans | | | |
| Example 436 | 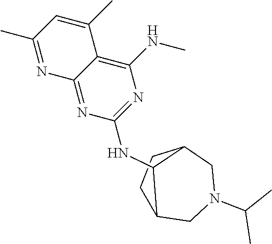 [SMILES: CNc1nc(NC2C3CCC2CN(C(C)C)C3)nc2nc(C)cc(C)c12] | Example 219, using intermediate from Example 227 and appropriate amine. Purified by HPLC^{Library} R_{T} = 1.72 min. | | Library C: 355 [M + H]+ |
| | Isomer 2 - cis or trans | | | |
| Example 437 | 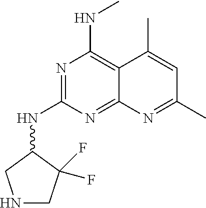 [SMILES: CNc1nc(NC2CNCC2(F)F)nc2nc(C)cc(C)c12] | Example 219, using intermediate from Example 227 and appropriate amine. Deprotection as per Example 140 Purified by SFC^{Library E} R_{T} = 7.60 | | Library C: 309 [M + H]+ |
| | Isomer 1 - The stereochemistry | min. | | |
| | has not been unambiguously | | | |
| | confirmed | | | |
| Example 438 | 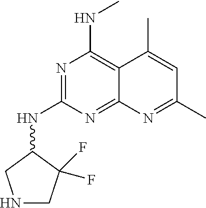 [SMILES: CNc1nc(NC2CNCC2(F)F)nc2nc(C)cc(C)c12] | Example 219, using intermediate from Example 227 and appropriate amine. Deprotection as per Example 140 Purified by SFC^{Library E} R_{T} = 3.65 | | Library C: 309 [M + H]+ |
| | Isomer 2 - The stereochemistry | min. | | |
| | has not been unambiguously | | | |
| | confirmed | | | |
| Example 439 | 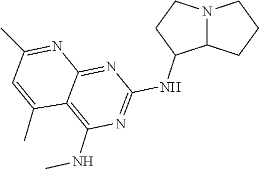 [SMILES: CNc1nc(NC2CCN3CCCC23)nc2nc(C)cc(C)c12] | Example 219, using intermediate from Example 227 and appropriate amine. | | Library I: 313 [M + H]+ |
| Example 440 | 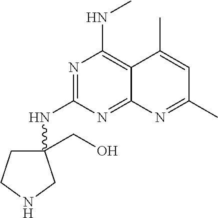 [SMILES: CNc1nc(NC2(CO)CCNC2)nc2nc(C)cc(C)c12] | Example 219, using intermediate from Example 227 and appropriate amine. Deprotection as per Example 140 Purified by SFC^{Library C} R_{T} = 7.21 min. | | Library C: 303 [M + H]+ |
| | Isomer 1 - The stereochemistry | | | |
| | has not been unambiguously | | | |
| | confirmed | | | |
| Example 441 | 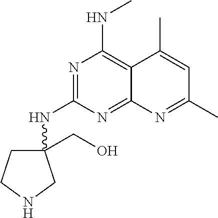 [SMILES: CNc1nc(NC2(CO)CCNC2)nc2nc(C)cc(C)c12] | Example 219, using intermediate from Example 227 and appropriate amine. Deprotection as per Example 140 Purified by SFC^{Library} R_{T} = 5.27 min. | | Library C: 303 [M + H]+ |
| | Isomer 2 - The stereochemistry | | | |
| | confirmed | | | |
| Example 442 | 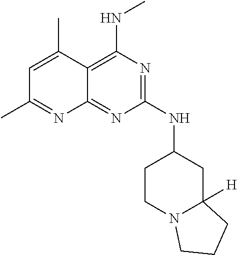 [SMILES: CNc1nc(NC2CCN3CCCC3C2)nc2nc(C)cc(C)c12] | Example 219, using intermediate from Example 227 and appropriate amine. Purified by SFC^{Library A} R_{T} = 11.50 min. | | Library C: 327 [M + H]+ |
| | Isomer 1 - The stereochemistry | | | |
| | has not been unambiguously | | | |
| | confirmed | | | |
| Example 443 | 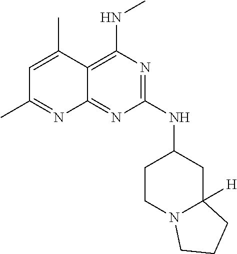 [SMILES: CNc1nc(NC2CCN3CCCC3C2)nc2nc(C)cc(C)c12] | Example 219, using intermediate from Example 227 and appropriate amine. Purified by SFC^{Library A} R_{T} = 9.80 min. | | Library C: 327 [M + H]+ |
| | Isomer 2 - The stereochemistry | | | |
| | has not been unambiguously | | | |
| | confirmed | | | |
| Example 444 | 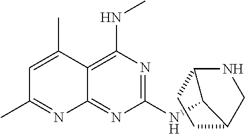 | Example 219, using intermediate from Example 227 and appropriate amine. Deprotection as per | | Library C: 299 [M + H]+ |
| | Mixture of cis isomers | Example 140 | | |
| Example 445 | 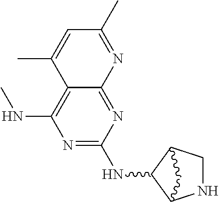 | Example 219, using intermediate from Example 227 and appropriate amine. Deprotection as per Example 140 Purified by SFC^{Library D} R_{T} = 7.03 | | Library C: 285 [M + H]+ |
| | Isomer 1 - The stereochemistry | min. | | |
| | has not been unambiguously | | | |
| | confirmed | | | |
| Example 446 | 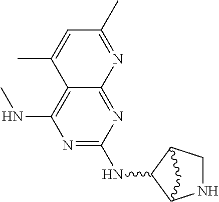 | Example 219, using intermediate from Example 227 and appropriate amine. Deprotection as per Example 140 Purified by SFC^{Library D} R_{T} = 7.27 | | Library C: 285 [M + H]+ |
| | Isomer 2 - The stereochemistry | min. | | |
| | has not been unambiguously | | | |
| | confirmed | | | |
| Example 447 | 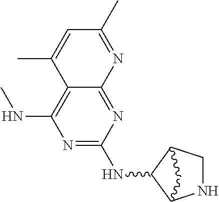 | Example 219, using intermediate from Example 227 and appropriate amine. Deprotection as per Example 140 Purified by SFC^{Library D} R_{T} = 7.87 | | Library C: 285 [M + H]+ |
| | Isomer 3 - The stereochemistry | min. | | |
| | has not been unambiguously | | | |
| | confirmed | | | |
| Example 448 | 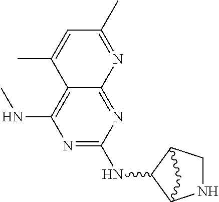 | Example 219, using intermediate from Example 227 and appropriate amine. Deprotection as per Example 140 Purified by SFC^{Library D} R_{T} = 10.20 | | Library C: 285 [M + H]+ |
| | Isomer 4 - The stereochemistry | min. | | |
| | has not been unambiguously | | | |
| | confirmed | | | |
| Example 449 | 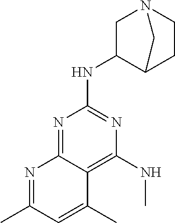 [SMILES: CNc1nc(NC2CN3CCC2C3)nc2nc(C)cc(C)c12] | Example 219, using intermediate from Example 227 and appropriate amine. | | Library C: 299 [M + H]+ |
| Example 450 | 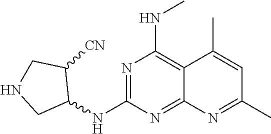 [SMILES: CNc1nc(NC2CNCC2C#N)nc2nc(C)cc(C)c12] | Example 219, using intermediate from Example 227 and appropriate amine. Deprotection as per | | Library C: 298 [M + H]+ |
| | Isomer 1 - The stereochemistry | Example 140 | | |
| | has not been unambiguously | Purified by | | |
| | confirmed | SFC^{Library A} | | |
| | | R_{T} = 6.00 | | |
| | | min. | | |
| Example 451 | 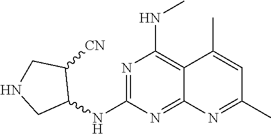 [SMILES: CNc1nc(NC2CNCC2C#N)nc2nc(C)cc(C)c12] | Example 219, using intermediate from Example 227 and appropriate amine. Deprotection as per | | Library C: 298 [M + H]+ |
| | Isomer 2 - The stereochemistry | Example 140 | | |
| | has not been unambiguously | Purified by | | |
| | confirmed | SFC^{Library A} | | |
| | | R_{T} = 7.80 | | |
| | | min. | | |
| Example 452 | 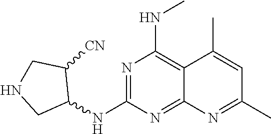 [SMILES: CNc1nc(NC2CNCC2C#N)nc2nc(C)cc(C)c12] | Example 219, using intermediate from Example 227 and appropriate amine. Deprotection as per | | Library C: 298 [M + H]+ |
| | Isomer 3 - The stereochemistry | Example 140 | | |
| | has not been unambiguously | Purified by | | |
| | confirmed | SFC^{Library A} | | |
| | | R^{T} = 10.60 | | |
| | | min. | | |
| Example 453 | 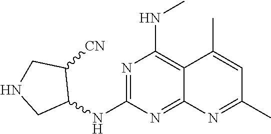 [SMILES: CNc1nc(NC2CNCC2C#N)nc2nc(C)cc(C)c12] | Example 219, using intermediate from Example 227 and appropriate amine. Deprotection as per | | Library C: 298 [M + H]+ |
| | Isomer 4 - The stereochemistry | Example 140 | | |
| | has not been unambiguously | Purified by | | |
| | confirmed | SFC^{Library} | | |
| | | R_{T} = 30.70 | | |
| | | min. | | |
| Example 454 | 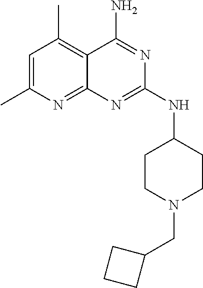 [SMILES: Cc1cc(C)c2c(N)nc(NC3CCN(CC4CCC4)CC3)nc2n1] | Example 426, using appropriate aldehyde | | Library C: 341 [M + H]⁺ |
| Example 455 | 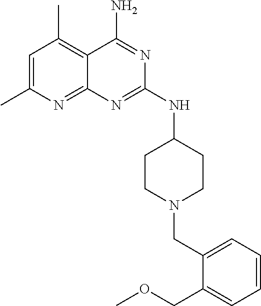 [SMILES: COCc1ccccc1CN1CCC(Nc2nc(N)c3c(C)cc(C)nc3n2)CC1] | Example 426, using appropriate aldehyde | | Library C: 407 [M + H]+ |
| Example 456 | 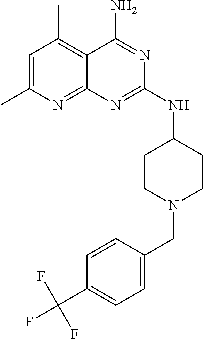 [SMILES: Cc1cc(C)c2c(N)nc(NC3CCN(Cc4ccc(C(F)(F)F)cc4)CC3)nc2n1] | Example 426, using appropriate aldehyde | | Library C: 431 [M + H]+ |
| Example 457 | 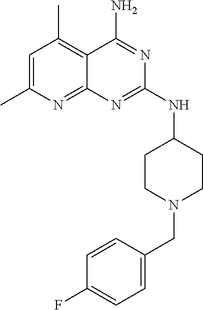 [SMILES: Cc1cc(C)c2c(N)nc(NC3CCN(Cc4ccc(F)cc4)CC3)nc2n1] | Example 426, using appropriate aldehyde | | Library C: 381 [M + H]+ |
| Example 458 | 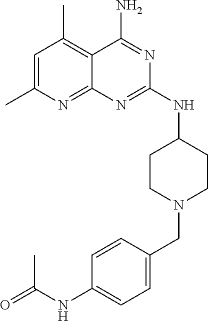 [SMILES: CC(=O)Nc1ccc(CN2CCC(Nc3nc(N)c4c(C)cc(C)nc4n3)CC2)cc1] | Example 426, using appropriate aldehyde | | Library C: 420 [M + H]+ |
| Example 459 | 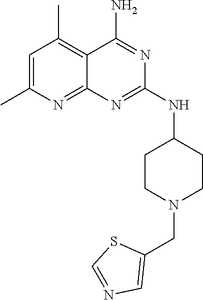 [SMILES: Cc1cc(C)c2c(N)nc(NC3CCN(Cc4cncs4)CC3)nc2n1] | Example 426, using appropriate aldehyde | | Library C: 370 [M + H]+ |
| Example 460 | 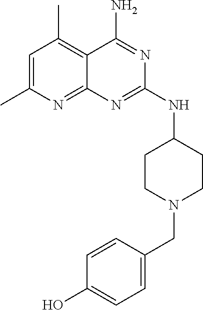 [SMILES: Cc1cc(C)c2c(N)nc(NC3CCN(Cc4ccc(O)cc4)CC3)nc2n1] | Example 426, using appropriate aldehyde | | Library E: 379 [M + H]+ |
| Example 461 | 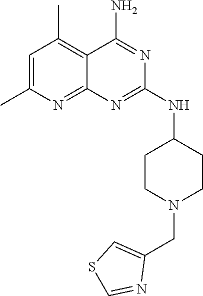 [SMILES: Cc1cc(C)c2c(N)nc(NC3CCN(Cc4cscn4)CC3)nc2n1] | Example 426, using appropriate aldehyde | | Library C: 370 [M + H]+ |
| Example 462 | 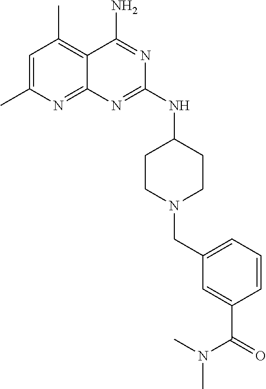 [SMILES: Cc1cc(C)c2c(N)nc(NC3CCN(Cc4cccc(C(=O)N(C)C)c4)CC3)nc2n1] | Example 426, using appropriate aldehyde | | Library C: 434 [M + H]+ |
| Example 463 | 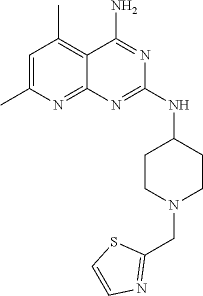 [SMILES: Cc1cc(C)c2c(N)nc(NC3CCN(Cc4nccs4)CC3)nc2n1] | Example 426, using appropriate aldehyde | | Library C: 370 [M + H]+ |
| Example 464 | 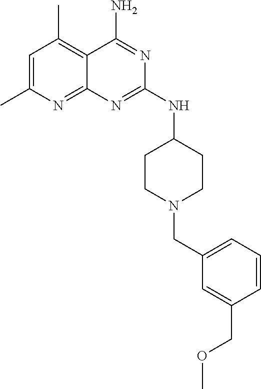 [SMILES: COCc1cccc(CN2CCC(Nc3nc(N)c4c(C)cc(C)nc4n3)CC2)c1] | Example 426, using appropriate aldehyde | | Library C: 407 [M + H]+ |
| Example 465 | 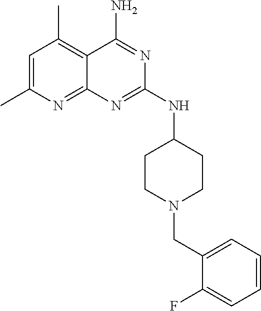 [SMILES: Cc1cc(C)c2c(N)nc(NC3CCN(Cc4ccccc4F)CC3)nc2n1] | Example 426, using appropriate aldehyde | | Library C: 381 [M + H]+ |
| Example 466 | 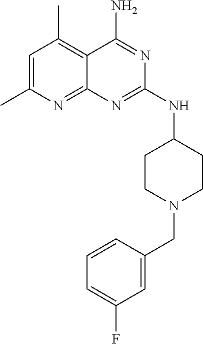 [SMILES: Cc1cc(C)c2c(N)nc(NC3CCN(Cc4cccc(F)c4)CC3)nc2n1] | Example 426, using appropriate aldehyde | | Library C: 381 [M + H]+ |
| Example 467 | 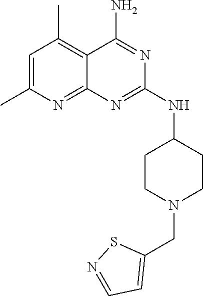 [SMILES: Cc1cc(C)c2c(N)nc(NC3CCN(Cc4ccns4)CC3)nc2n1] | Example 426, using appropriate aldehyde | | Library C: 370 [M + H]+ |
| Example 468 | 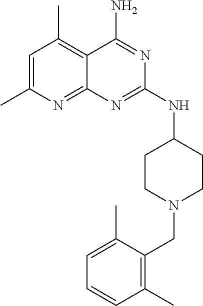 [SMILES: Cc1cc(C)c2c(N)nc(NC3CCN(Cc4c(C)cccc4C)CC3)nc2n1] | Example 426, using appropriate aldehyde | | Library C: 391 [M + H]+ |
| Example 469 | 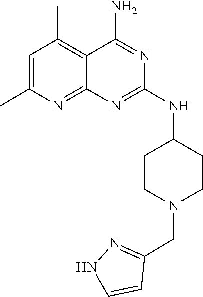 [SMILES: Cc1cc(C)c2c(N)nc(NC3CCN(Cc4cc[nH]n4)CC3)nc2n1] | Example 426, using appropriate aldehyde | | Library C: 353 [M + H]+ |
| Example 470 | 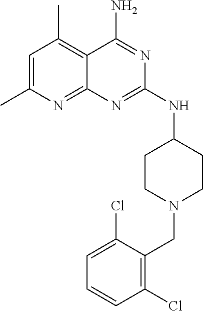 [SMILES: Cc1cc(C)c2c(N)nc(NC3CCN(Cc4c(Cl)cccc4Cl)CC3)nc2n1] | Example 426, using appropriate aldehyde | | Library C: 431 [M + H]+ |
| Example 471 | 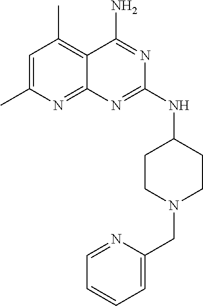 [SMILES: Cc1cc(C)c2c(N)nc(NC3CCN(Cc4ccccn4)CC3)nc2n1] | Example 426, using appropriate aldehyde | | Library C: 364 [M + H]+ |
| Example 472 | 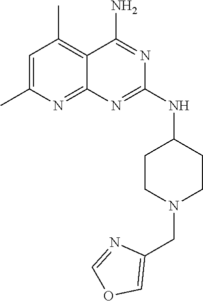 [SMILES: Cc1cc(C)c2c(N)nc(NC3CCN(Cc4cocn4)CC3)nc2n1] | Example 426, using appropriate aldehyde | | Library C: 354 [M + H]+ |
| Example 473 | 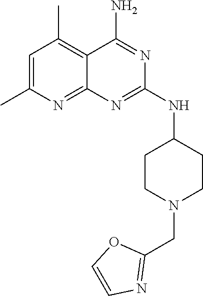 [SMILES: Cc1cc(C)c2c(N)nc(NC3CCN(Cc4ncco4)CC3)nc2n1] | Example 426, using appropriate aldehyde | | Library C: 354 [M + H]+ |
| Example 474 | 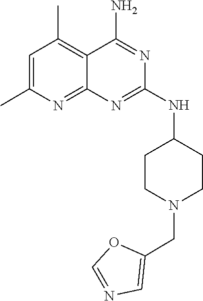 [SMILES: Cc1cc(C)c2c(N)nc(NC3CCN(Cc4cnco4)CC3)nc2n1] | Example 426, using appropriate aldehyde | | Library C: 354 [M + H]+ |
| Example 475 | 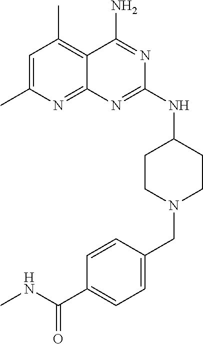 [SMILES: CNC(=O)c1ccc(CN2CCC(Nc3nc(N)c4c(C)cc(C)nc4n3)CC2)cc1] | Example 426, using appropriate aldehyde | | Library C: 420 [M + H]+ |
| Example 476 | 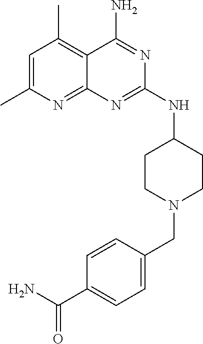 [SMILES: Cc1cc(C)c2c(N)nc(NC3CCN(Cc4ccc(C(N)=O)cc4)CC3)nc2n1] | Example 426, using appropriate aldehyde | | Library C: 406 [M + H]+ |
| Example 477 | 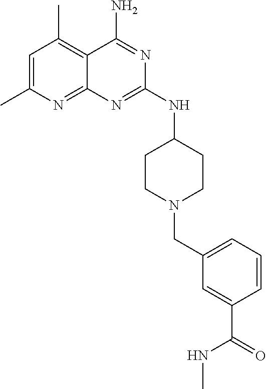 [SMILES: CNC(=O)c1cccc(CN2CCC(Nc3nc(N)c4c(C)cc(C)nc4n3)CC2)c1] | Example 426, using appropriate aldehyde | | Library C: 420 [M + H]+ |
| Example 478 | 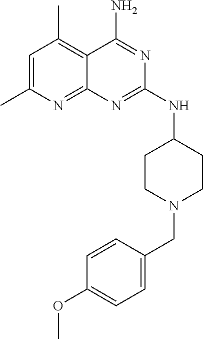 [SMILES: COc1ccc(CN2CCC(Nc3nc(N)c4c(C)cc(C)nc4n3)CC2)cc1] | Example 426, using appropriate aldehyde | | Library C: 393 [M + H]+ |
| Example 479 | 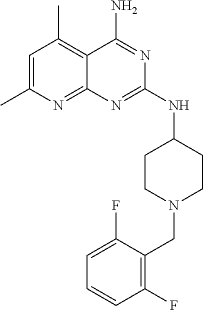 [SMILES: Cc1cc(C)c2c(N)nc(NC3CCN(Cc4c(F)cccc4F)CC3)nc2n1] | Example 426, using appropriate aldehyde | | Library C: 399 [M + H]+ |
| Example 480 | 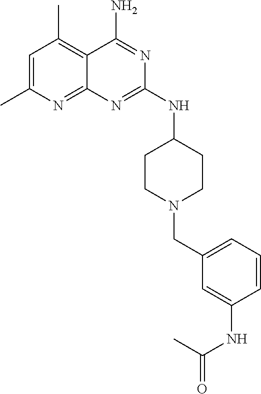 [SMILES: CC(=O)Nc1cccc(CN2CCC(Nc3nc(N)c4c(C)cc(C)nc4n3)CC2)c1] | Example 426, using appropriate aldehyde | | Library C: 420 [M + H]+ |
| Example 481 | 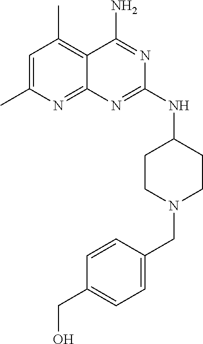 [SMILES: Cc1cc(C)c2c(N)nc(NC3CCN(Cc4ccc(CO)cc4)CC3)nc2n1] | Example 426, using appropriate aldehyde | | Library C: 393 [M + H]+ |
| Example 482 | 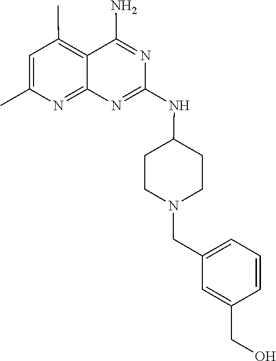 [SMILES: Cc1cc(C)c2c(N)nc(NC3CCN(Cc4cccc(CO)c4)CC3)nc2n1] | Example 426, using appropriate aldehyde | | Library C: 393 [M + H]+ |
| Example 483 | 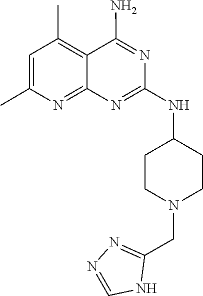 [SMILES: Cc1cc(C)c2c(N)nc(NC3CCN(Cc4nnc[nH]4)CC3)nc2n1] | Example 426, using appropriate aldehyde | | Library C: 354 [M + H]+ |
| Example 484 | 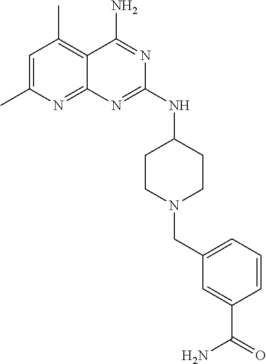 [SMILES: Cc1cc(C)c2c(N)nc(NC3CCN(Cc4cccc(C(N)=O)c4)CC3)nc2n1] | Example 426, using appropriate aldehyde | | Library C: 406 [M + H]+ |
| Example 485 | 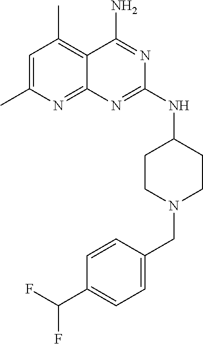 [SMILES: Cc1cc(C)c2c(N)nc(NC3CCN(Cc4ccc(C(F)F)cc4)CC3)nc2n1] | Example 426, using appropriate aldehyde | | Library C: 413 [M + H]+ |
| Example 486 | 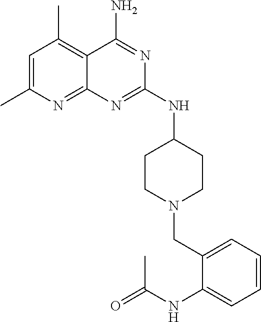 [SMILES: CC(=O)Nc1ccccc1CN1CCC(Nc2nc(N)c3c(C)cc(C)nc3n2)CC1] | Example 426, using appropriate aldehyde | | Library C: 420 [M + H]+ |
| Example 487 | 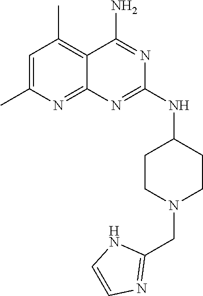 [SMILES: Cc1cc(C)c2c(N)nc(NC3CCN(Cc4ncc[nH]4)CC3)nc2n1] | Example 426, using appropriate aldehyde | | Library C: 353 [M + H]+ |
| Example 488 | 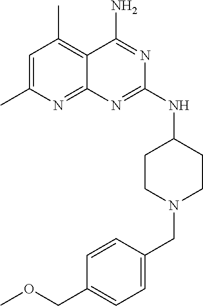 [SMILES: COCc1ccc(CN2CCC(Nc3nc(N)c4c(C)cc(C)nc4n3)CC2)cc1] | Example 426, using appropriate aldehyde | | Library C: 407 [M + H]+ |
| Example 489 | 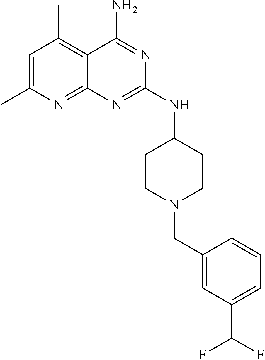 [SMILES: Cc1cc(C)c2c(N)nc(NC3CCN(Cc4cccc(C(F)F)c4)CC3)nc2n1] | Example 426, using appropriate aldehyde | | Library C: 413 [M + H]+ |
| Example 490 | 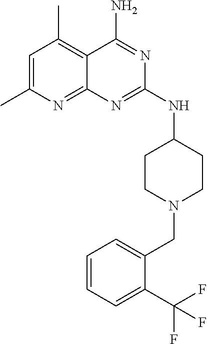 [SMILES: Cc1cc(C)c2c(N)nc(NC3CCN(Cc4ccccc4C(F)(F)F)CC3)nc2n1] | Example 426, using appropriate aldehyde | | Library C: 431 [M + H]+ |
| Example 491 | 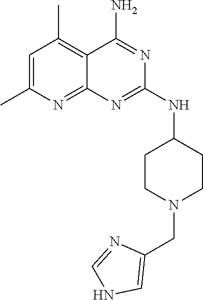 [SMILES: Cc1cc(C)c2c(N)nc(NC3CCN(Cc4c[nH]cn4)CC3)nc2n1] | Example 426, using appropriate aldehyde | | Library C: 353 [M + H]+ |
| Example 492 | 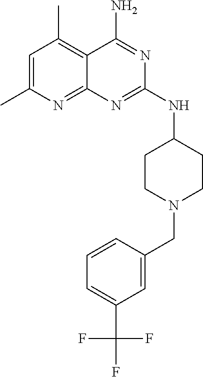 [SMILES: Cc1cc(C)c2c(N)nc(NC3CCN(Cc4cccc(C(F)(F)F)c4)CC3)nc2n1] | Example 426, using appropriate aldehyde | | Library C: 431 [M + H]+ |
| Example 493 | 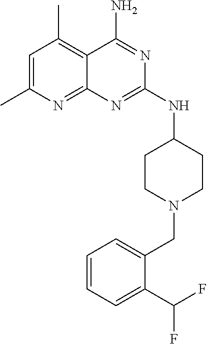 [SMILES: Cc1cc(C)c2c(N)nc(NC3CCN(Cc4ccccc4C(F)F)CC3)nc2n1] | Example 426, using appropriate aldehyde | | Library C: 413 [M + H]+ |
| Example 494 | 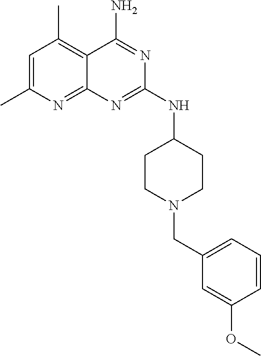 [SMILES: COc1cccc(CN2CCC(Nc3nc(N)c4c(C)cc(C)nc4n3)CC2)c1] | Example 426, using appropriate aldehyde | | Library C: 393 [M + H]+ |
| Example 495 | 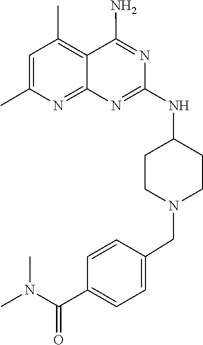 [SMILES: Cc1cc(C)c2c(N)nc(NC3CCN(Cc4ccc(C(=O)N(C)C)cc4)CC3)nc2n1] | Example 426, using appropriate aldehyde | | Library C: 434 [M + H]+ |
| Example 496 | 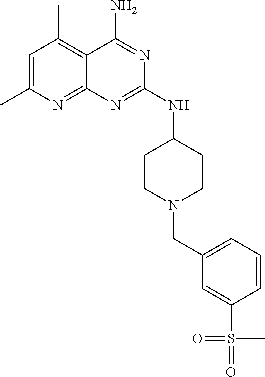 | Example 426, using appropriate aldehyde | | Library C: 441 [M + H]+ |
| Example 497 | 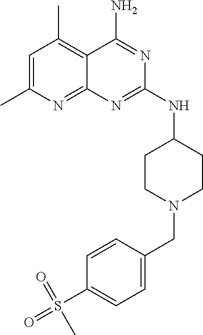 [SMILES: Cc1cc(C)c2c(N)nc(NC3CCN(Cc4ccc(S(C)(=O)=O)cc4)CC3)nc2n1] | Example 426, using appropriate aldehyde | | Library C: 441 [M + H]+ |
| Example 498 | 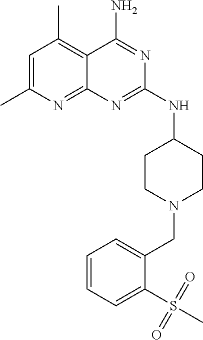 [SMILES: Cc1cc(C)c2c(N)nc(NC3CCN(Cc4ccccc4S(C)(=O)=O)CC3)nc2n1] | Example 426, using appropriate aldehyde | | Library C: 441 [M + H]+ |
| Example 499 | 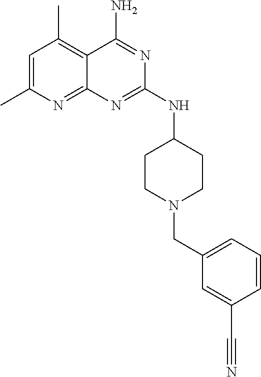 [SMILES: Cc1cc(C)c2c(N)nc(NC3CCN(Cc4cccc(C#N)c4)CC3)nc2n1] | Example 426, using appropriate aldehyde | | Library C: 388 [M + H]+ |
| Example 500 | 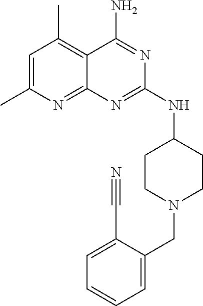 [SMILES: Cc1cc(C)c2c(N)nc(NC3CCN(Cc4ccccc4C#N)CC3)nc2n1] | Example 426, using appropriate aldehyde | | Library C: 388 [M + H]+ |
| Example 501 | 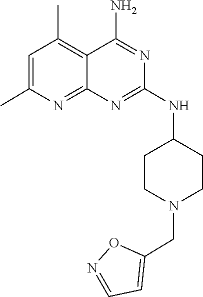 [SMILES: Cc1cc(C)c2c(N)nc(NC3CCN(Cc4ccno4)CC3)nc2n1] | Example 426, using appropriate aldehyde | | Library C: 354 [M + H]+ |
| Example 502 | 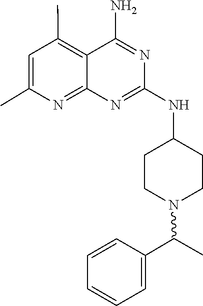 [SMILES: Cc1cc(C)c2c(N)nc(NC3CCN(C(C)c4ccccc4)CC3)nc2n1] | Example 426, using appropriate ketone Purified by SFC^{Library E} R_{T} = 9.84 min. | | Library C: 377 [M + H]+ |
| | Isomer 1 - The stereochemistry | | | |
| | has not been unambiguously | | | |
| | confirmed | | | |
| Example 503 | 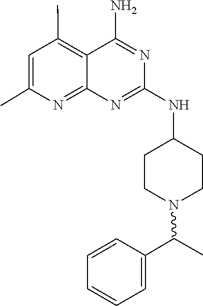 [SMILES: Cc1cc(C)c2c(N)nc(NC3CCN(C(C)c4ccccc4)CC3)nc2n1] | Example 426, using appropriate ketone Purified by SFC^{Library E} R_{T} = 5.97 min. | | Library C: 377 [M + H]+ |
| | Isomer 2 - The stereochemistry | | | |
| | has not been unambiguously | | | |
| | confirmed | | | |
| Example 504 | 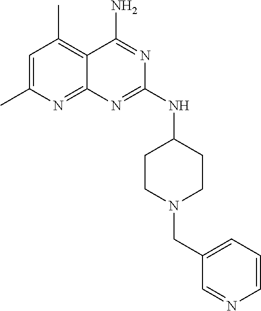 [SMILES: Cc1cc(C)c2c(N)nc(NC3CCN(Cc4cccnc4)CC3)nc2n1] | Example 426, using appropriate aldehyde | | Library C: 364 [M + H]+ |
| Example 505 | 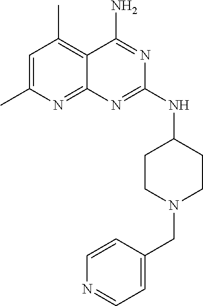 [SMILES: Cc1cc(C)c2c(N)nc(NC3CCN(Cc4ccncc4)CC3)nc2n1] | Example 426, using appropriate aldehyde | | Library C: 364 [M + H]+ |
| Example 506 | 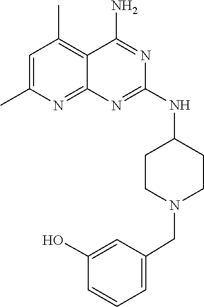 [SMILES: Cc1cc(C)c2c(N)nc(NC3CCN(Cc4cccc(O)c4)CC3)nc2n1] | Example 426, using appropriate aldehyde | | Library C: 379 [M + H]+ |

### Example 507: 4-((4-((4-amino-5,7-dimethylpyrido[2,3-d]pyrimidin-2-yl)amino)piperidin-1-yl)methyl-d₂)benzonitrile

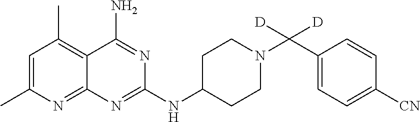 [SMILES: [2H]C([2H])(c1ccc(C#N)cc1)N1CCC(Nc2nc(N)c3c(C)cc(C)nc3n2)CC1]

### Preparation 179—4-(hydroxymethyl-d₂)benzonitrile

To a stirred solution of methyl 4-cyanobenzoate (500 mg, 3.10 mmol) in THF (5.00 mL) and CD₃OD (5.00 mL) was added sodium tetrahydroborate-d₄ (520 mg, 12.4 mmol) and CaCl₂) (689 mg, 6.21 mmol) at 0° C. The resulting mixture was stirred at 0° C. for 3 h. The reaction was quenched with water/Ice at 0° C. The resulting mixture was extracted with EtOAc (3×50 mL). The combined organic layers were dried over anhydrous Na₂SO₄. After filtration, the filtrate was concentrated under reduced pressure. The residue was purified by silica gel column chromatography, eluted with PE/EA (3:2) to afford the title compound (330 mg, 78.7%) as an off-white oil. LCMS m/z=269 [2M−H]—.

### Preparation 180—4-(bromomethyl-d₂)benzonitrile

To a stirred solution of 4-(hydroxymethyl-d₂)benzonitrile (276 mg, 2.04 mmol) in DCM (5.50 mL) at 0° C. was added PBr₃ (193 μL, 0.713 mmol). The resulting mixture was stirred at 0° C. for 1 h. The reaction was quenched with water/Ice at 0° C. The resulting mixture was extracted with CH₂Cl₂ (3×30 mL). The combined organic layers were washed with brine, dried over anhydrous Na₂SO₄. After filtration, the filtrate was concentrated under reduced pressure. The residue was purified by silica gel column chromatography, eluted with PE/EA (5:1) to afford the title compound (200 mg, 49.4%) as an off-white solid. ¹H NMR (400 MHz, DMSO-d₆) δ 7.84 (d, J=8.0 Hz, 2H), 7.65 (d, J=8.1 Hz, 2H).

### Preparation 181—4-((4-((4-amino-5,7-dimethylpyrido[2,3-d]pyrimidin-2-yl)amino)piperidin-1-yl)methyl-d₂)benzonitrile

To a stirred solution of Example 279 (138 mg, 0.505 mmol) in DMF (2.00 mL) at room temperature was added Cs₂CO₃ (329 mg, 1.01 mmol). The resulting mixture was stirred at 100° C. for 1 h. The mixture was allowed to cool down to room temperature and filtered through a short pad of Celite. The filter cake was washed with DMF (2×0.5 mL). The combined filtrate was purified by Prep-HPLC^{BC} to afford the title compound (93.4 mg, 47.5%, >97% D) as a light yellow solid. ¹H NMR (400 MHz, DMSO-d₆) δ 7.85-7.73 (m, 2H), 7.59-7.46 (m, 2H), 6.94-6.53 (m, 3H), 6.38 (s, 1H), 3.90-3.71 (m, 1H), 2.86-2.74 (m, 2H), 2.63 (s, 3H), 2.37 (s, 3H), 2.17-2.01 (m, 2H), 1.83-1.80 (m, 2H), 1.56-1.47 (m, 2H). LCMS^{W} m/z=390 [M+H]⁺

### Example 508: 4-((4-((4-amino-5,7-dimethylpyrido[2,3-d]pyrimidin-2-yl)amino)piperidin-1-yl)methyl)-3-(difluoromethyl)benzonitrile

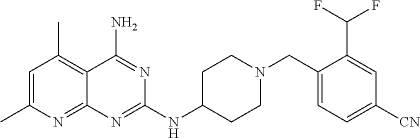 [SMILES: Cc1cc(C)c2c(N)nc(NC3CCN(Cc4ccc(C#N)cc4C(F)F)CC3)nc2n1]

### Preparation 182—4-bromo-3-(difluoromethyl)benzonitrile

To a stirred solution of 4-bromo-3-formylbenzonitrile (2.00 g, 9.52 mmol) in DCM (120 mL) was added bis(2-methoxyethyl)aminosulfur trifluoride (5.28 mL, 28.7 mmol) at room temperature. The resulting mixture was stirred at 40° C. for 1 h. The reaction was quenched by the addition of sat. NaHCO₃ (aq., 300 mL) at 0° C. The resulting mixture was extracted with EtOAc (3×300 mL). The combined organic layers were washed with brine (300 mL), dried over anhydrous Na₂SO₄. After filtration, the filtrate was concentrated under reduced pressure. The residue was purified by silica gel column chromatography, eluted with PE/EA (10:1) to afford the title compound (1.80 g, 81.5%) as a white solid. ¹H NMR (400 MHz, Chloroform-d) δ 7.94 (d, J=2.0 Hz, 1H), 7.77 (dt, J=8.3, 1.2 Hz, 1H), 7.68-7.59 (m, 1H), 6.89 (t, J=54.3 Hz, 1H).

### Preparation 183—methyl 4-cyano-2-(difluoromethyl)benzoate

To a pressure tank were added 4-bromo-3-(difluoromethyl)benzonitrile (1.00 g, 4.31 mmol), MeOH (18.0 mL), DMF (2.00 mL), Pd(dppf)Cl₂ (320 g, 0.431 mmol) and Et₃N (870 g, 8.62 mmol). The mixture was purged with nitrogen for 3 min and then was pressurized to 15.0 atm with carbon monoxide. The resulting mixture was stirred at 100° C. overnight. The reaction mixture was cooled to room temperature and concentrated under reduced pressure. The residue was purified by silica gel column chromatography, eluted with PE/EA (10:1) to afford the title compound (900 mg, 98.9%) as a white solid. LCMS m/z=212 [M+H]⁺.

### Preparation 184—3-(difluoromethyl)-4-(hydroxymethyl)benzonitrile

To a stirred mixture of methyl 4-cyano-2-(difluoromethyl)benzoate (900 mg, 4.35 mmol) and CaCl₂) (967 mg, 8.71 mmol) in EtOH (10.0 mL) and THF (10.0 mL) was added NaBH₄ (659 mg, 17.4 mmol) at room temperature. The resulting mixture was stirred at room temperature for 1 h. The reaction was quenched by the addition of sat. NH₄Cl (aq.) (50 mL) at 0° C. The resulting mixture was extracted with EtOAc (3×50 mL). The combined organic layers were washed with brine (50 mL), dried over anhydrous Na₂SO₄. After filtration, the filtrate was concentrated under reduced pressure to afford the title compound (760 mg, crude) as a yellow solid. The crude product was used in the next step directly without further purification. LCMS m/z=184 [M+H]⁺.

### Preparation 185—3-(difluoromethyl)-4-formylbenzonitrile

To a stirred solution of 3-(difluoromethyl)-4-(hydroxymethyl)benzonitrile (710 mg, 3.88 mmol) in DCM (14.0 mL) was added Dess-Martin reagent (3.29 g, 7.75 mmol) in portions at room temperature. The resulting mixture was stirred at room temperature for 1 h. The reaction was quenched by the addition of sat. NaHCO₃ (aq.) (100 mL) at 0° C. The resulting mixture was extracted with CH₂Cl₂ (3×100 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (10:1) to afford the title compound (480 mg, 68.4%) as a white solid. LCMS m/z=180 [M−H]⁻.

### Preparation 186—4-((4-((4-amino-5,7-dimethylpyrido[2,3-d]pyrimidin-2-yl)amino)piperidin-1-yl)methyl)-3-(difluoromethyl)benzonitrile

A solution of Example 279 (100 mg, 0.367 mmol) in DCE (3.00 mL) was treated with 3-(difluoromethyl)-4-formylbenzonitrile (133 mg, 0.734 mmol) at 40° C. for 1 h and followed by the addition of sodium bis(acetyloxy)boranuidyl acetate (156 mg, 0.734 mmol) at room temperature. The resulting mixture was stirred at 40° C. for 1 h. The mixture was allowed to cool down to room temperature. The resulting mixture was diluted with water (50.0 mL). The aqueous layer was extracted with EtOAc (3×50 mL). The combined organic layers were concentrated under reduced pressure. The residue was purified by Prep-HPLC^{AK} to afford the title compound (53.9 mg, 33.6%) as a white solid. ¹H NMR (400 MHz, DMSO-d₆) δ 8.05 (s, 1H), 7.99 (d, J=4.0 Hz, 1H), 7.72 (d, J=8.0 Hz, 1H), 7.41 (t, J=54.6 Hz, 1H), 6.94-6.58 (m, 3H), 6.38 (s, 1H), 3.93-3.75 (m, 1H), 3.69 (s, 2H), 2.76 (d, J=11.3 Hz, 2H), 2.63 (s, 3H), 2.37 (s, 3H), 2.11 (t, J=11.3 Hz, 2H), 1.82 (d, J=12.0 Hz, 2H), 1.57-1.43 (m, 2H). LCMS^{S} m/z=438 [M+H]⁺.

### Example 509: (3S,4S)-1-(methyl-d₃)-4-((5-methyl-4-((methyl-da)amino)-7,8-dihydro-6H-cyclopenta[5,6]pyrido[2,3-d]pyrimidin-2-yl)amino)pyrrolidin-3-ol

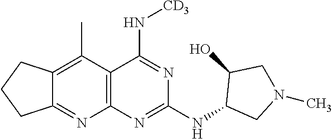 [SMILES: [2H]C([2H])([2H])Nc1nc(N[C@H]2CN(C)C[C@@H]2O)nc2nc3c(c(C)c12)CCC3]

### Preparation 187—2-chloro-5-methyl-N-(methyl-d3)-7,8-dihydro-6H-cyclopenta[5,6]pyrido[2,3-d]pyrimidin-4-amine

To a stirring mixture of 2,4-dichloro-5-methyl-7,8-dihydro-6H-cyclopenta[5,6]pyrido[2,3-d]pyrimidine (4.50 g, 17.8 mmol) (Example 230, Preparation 114) and DIEA (4.58 g, 35.4 mmol) in THF (100 mL) at room temperature was added methan-d₃-amine hydrochloride (2.00 g, 28.3 mmol). The resulting mixture was stirred at room temperature for overnight. The precipitated solids were collected by filtration and washed with THF (3×20 mL). This resulted in the title compound as brown solid (2.70 g crude). LCMS m/z=252 [M+H]⁺.

### Preparation 188—tert-butyl (3S,4S)-3-hydroxy-4-((5-methyl-4-((methyl-d3)amino)-7,8-dihydro-6H-cyclopenta[5,6]pyrido[2,3-d]pyrimidin-2-yl)amino)pyrrolidine-1-carboxylate

A mixture of 2-chloro-5-methyl-N-(methyl-da)-7,8-dihydro-6H-cyclopenta[5,6]pyrido[2,3-d]pyrimidin-4-amine (500 mg, 1.99 mmol) and tert-butyl (3S,4S)-3-amino-4-hydroxypyrrolidine-1-carboxylate (604 mg, 3.00 mmol) in 1,4-dioxane (10.0 mL) was stirred at 100° C. overnight. The mixture was allowed to cool down to room temperature. The resulting mixture was concentrated under reduced pressure. The residue was purified by Prep-HPLC^{A}w to afford the title compound as brown yellow solid (460 mg, 55.4%). LCMS m/z=418 [M+H]⁺.

### Preparation 189—(3S,4S)-1-(methyl-d₃)-4-((5-methyl-4-((methyl-d₃)amino)-7,8-dihydro-6H-cyclopenta[5,6]pyrido[2,3-d]pyrimidin-2-yl)amino)pyrrolidin-3-ol

To a stirred solution of tert-butyl (3S,4S)-3-hydroxy-4-((5-methyl-4-((methyl-d₃)amino)-7,8-dihydro-6H-cyclopenta[5,6]pyrido[2,3-d]pyrimidin-2-yl)amino)pyrrolidine-1-carboxylate (100 mg, 0.240 mmol) in THF (3.00 mL) was added lithium aluminium deuteride (3.84 mL, 3.84 mmol, 1.0 M in THF) dropwise at 0° C. The resulting mixture was stirred at room temperature overnight. The reaction was quenched by the addition of MeOH (3.00 mL) at 0° C. The resulting mixture was concentrated under reduced pressure and purified by Prep-HPLC^{Y} to afford the title compound as an off-white solid (10.7 mg, 13.4%, >98% D, ee>99%). ¹H NMR (400 MHz, Methanol-d₄) δ 4.28-4.14 (m, 2H), 3.15-3.04 (m, 1H), 3.02-2.88 (m, 5H), 2.63 (s, 3H), 2.61-2.53 (m, 2H), 2.13 (p, J=7.7 Hz, 2H). LCMS^{AX} m/z=335 [M+H]⁺.

### Example 510: 2-((5-methyl-2-(piperidin-4-ylamino)-7,8-dihydro-6H-cyclopenta[5,6]pyrido[2,3-d]pyrimidin-4-yl)amino)ethan-1-ol

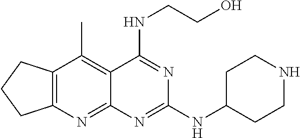 [SMILES: Cc1c2c(nc3nc(NC4CCNCC4)nc(NCCO)c13)CCC2]

### Preparation 190—N-(2-((tert-butyldimethylsilyl)oxy)ethyl)-2-chloro-5-methyl-7,8-dihydro-6H-cyclopenta[5,6]pyrido[2,3-d]pyrimidin-4-amine

A mixture of 2,4-dichloro-5-methyl-7,8-dihydro-6H-cyclopenta[5,6]pyrido[2,3-d]pyrimidine (1.00 g, 3.94 mmol) (Example 230, Preparation 114), DIEA (1.02 g, 7.87 mmol) and 2-((tert-butyldimethylsilyl)oxy)ethan-1-amine (1.04 g, 5.90 mmol) in THF (20.0 mL) was stirred at room temperature for 1 h. The resulting mixture was concentrated under reduced pressure and purified by silica gel column chromatography, eluted with PE/EA (1:1) to afford the title compound (550 mg, 35.7%) as off-white solid. LCMS m/z=393 [M+H]⁺.

### Preparation 191—tert-butyl 4-((4-((2-((tert-butyldimethylsilyl)oxy)ethyl)amino)-5-methyl-7,8-dihydro-6H-cyclopenta[5,6]pyrido[2,3-d]pyrimidin-2-yl)amino)piperidine-1-carboxylate

A solution of N-(2-((tert-butyldimethylsilyl)oxy)ethyl)-2-chloro-5-methyl-7,8-dihydro-6H-cyclopenta[5,6]pyrido[2,3-d]pyrimidin-4-amine (530 mg, 1.35 mmol) and tert-butyl 4-aminopiperidine-1-carboxylate (540 mg, 2.70 mmol) in DMSO (15.0 mL) was stirred at 120° C. for 3 days. The reaction was allowed to cool down to room temperature. The resulting mixture was purified by Prep-HPLC^{C} directly to afford the title compound (350 mg, 46.6%) as a yellow solid. LCMS m/z=557 [M+H]⁺.

### Preparation 192—2-((5-methyl-2-(piperidin-4-ylamino)-7,8-dihydro-6H-cyclopenta[5,6]pyrido[2,3-d]pyrimidin-4-yl)amino)ethan-1-ol

A solution of tert-butyl 4-((4-((2-((tert-butyldimethylsilyl)oxy)ethyl)amino)-5-methyl-7,8-dihydro-6H-cyclopenta[5,6]pyrido[2,3-d]pyrimidin-2-yl)amino)piperidine-1-carboxylate (350 mg, 0.628 mmol) in HCl (4.00 M in 1,4-dioxane, 3.00 mL) was stirred at room temperature for 1 h. The mixture was concentrated under reduced pressure. The residue was dissolved with MeOH (2.00 mL) and basified to pH 8 with Et₃N. The resulting mixture was purified by Prep-HPLC^{C} to afford the title compound (142 mg, 49.4%) as a white solid. ¹H NMR (400 MHz, DMSO-d₆) δ 6.59 (br, 1H), 6.43 (br, 1H), 4.81 (br, 1H), 3.91-3.76 (m, 1H), 3.69-3.60 (m, 2H), 3.59-3.48 (m, 2H), 2.96 (d, J=12.3 Hz, 2H), 2.86 (q, J=7.1, 6.7 Hz, 4H), 2.59 (s, 3H), 2.57-2.51 (m, 2H), 2.08-1.94 (m, 2H), 1.88-1.70 (m, 2H), 1.34-1.20 (m, 2H). LCMS^{A} m/z=343 [M+H]⁺.

### Example 511: N-(2-((4-((4-amino-5,7-dimethylpyrido[2,3-d]pyrimidin-2-yl)amino)piperidin-1-yl)methyl)-5-cyanophenyl)acetamide

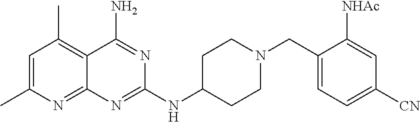 [SMILES: CC(=O)Nc1cc(C#N)ccc1CN1CCC(Nc2nc(N)c3c(C)cc(C)nc3n2)CC1]

### Preparation 193—methyl 2-acetamido-4-cyanobenzoate

To a stirred mixture of methyl 2-amino-4-cyanobenzoate (1.00 g, 5.68 mmol) and K₂CO₃ (1.57 g, 11.4 mmol) in THE (20.0 mL) was added acetyl chloride (532 mg, 6.82 mmol) at room temperature. The resulting mixture was stirred at room temperature for 2 h. The reaction was diluted with water (20 mL) and extracted with EtOAc (3×30 mL). The combined organic layers were washed with brine (2×20 mL), dried over anhydrous Na₂SO₄. After filtration, the filtrate was concentrated under reduced pressure to afford the title compound (1.10 g, crude) as an off-white solid. LCMS m/z=219 [M+H]⁺.

### Preparation 194—N-(5-cyano-2-(hydroxymethyl)phenyl)acetamide

To a stirred solution of methyl 2-acetamido-4-cyanobenzoate (500 mg, 2.29 mmol) in EtOH (10.0 mL) and THE (6.00 mL) were added CaCl₂ (509 mg, 4.58 mmol) and NaBH₄ (347 mg, 9.16 mmol) at room temperature. The reaction mixture was stirred at room temperature for 1 h. The reaction was quenched with sat. NH₄Cl (aq.) at 0° C. The resulting mixture was extracted with EtOAc (3×40 mL). The combined organic layers were washed with brine (3×5 mL), dried over anhydrous Na₂SO₄. After filtration, the filtrate was concentrated under reduced pressure. This resulted in the title compound as a light yellow solid (400 mg, crude). LCMS m/z=191 [M+H]⁺.

### Preparation 195—N-(5-cyano-2-formylphenyl)acetamide

A mixture of N-(5-cyano-2-(hydroxymethyl)phenyl)acetamide (330 mg, 1.74 mmol) and Dess-Martin reagent (1.47 g, 3.47 mmol) in DCM (6.00 mL) was stirred at room temperature for 1 h. The reaction was quenched with sat. NaHCO₃ (aq.) at room temperature. The resulting mixture was extracted with CH₂Cl₂ (3×50 mL). The combined organic layers were washed with brine (30 mL), dried over anhydrous Na₂SO₄. After filtration, the filtrate was concentrated under reduced pressure. The residue was purified by silica gel column chromatography, eluted with PE/EA (1:1) to afford the title compound as a yellow solid (190 mg, 58.2%). LCMS m/z=189 [M+H]⁺.

### Preparation 196—N-(2-((4-((4-amino-5,7-dimethylpyrido[2,3-d]pyrimidin-2-yl)amino)piperidin-1-yl)methyl)-5-cyanophenyl)acetamide

A solution of Example 279 (100 mg, 0.367 mmol) in EtOH (2.00 mL) was treated with N-(5-cyano-2-formylphenyl)acetamide (104 mg, 0.550 mmol) at 40° C. for 1 h followed by the addition of NaBH₄ (41.7 mg, 1.10 mmol) in portions at room temperature. The resulting mixture was stirred at room temperature for 1 h. The reaction was quenched with water/Ice at room temperature. The resulting mixture was concentrated under reduced pressure. The residue was purified by Prep-HPLC^{AO} to afford the title compound as a white solid (6.9 mg, 4.2%). ¹H NMR (400 MHz, DMSO-d₆) δ 11.22 (s, 1H), 8.45 (s, 1H), 7.56-7.32 (m, 2H), 7.02-6.59 (m, 3H), 6.50 (s, 1H), 3.97-3.80 (m, 1H), 3.73 (s, 2H), 2.89-2.74 (m, 2H), 2.64 (s, 3H), 2.37 (s, 3H), 2.25-2.07 (m, 5H), 1.97-1.79 (m, 2H), 1.65-1.43 (m, 2H). LCMS^{W} m/z=445 [M+H]⁺.

### Example 512: 5,7-dimethyl-N⁴-(methyl-da)-N²-(1-(methyl-da)piperidin-4-yl)pyrido[2,3-d]pyrimidine-2,4-diamine

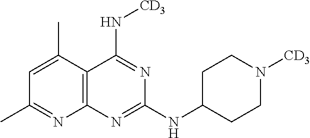 [SMILES: [2H]C([2H])([2H])Nc1nc(NC2CCN(C([2H])([2H])[2H])CC2)nc2nc(C)cc(C)c12]

A solution of Example 589 (100 mg, 0.346 mmol) and Et₃N (96.1 μL, 0.692 mmol) in 1,2-dichloroethane (2.00 mL) was treated with Formaldehyde-d₂ (20% wt in D₂O) (22.1 mg, 0.069 mmol) at room temperature for 1 h followed by the addition of sodium tetrahydroborate-d₄ (28.9 mg, 0.692 mmol) at room temperature. The resulting mixture was stirred at room temperature for another 1 h. The reaction was quenched by the addition of water/ice (20 mL) at 0° C. The aqueous layer was extracted with EtOAc (3×20 mL). The organic layers were concentrated under reduced pressure. The crude product was purified by Prep-HPLC^{AX} to afford the title compound (20.5 mg, 19.4%) as a white solid. ¹H NMR (400 MHz, DMSO-d₆) δ 6.86-6.60 (m, 2H), 6.51 (s, 1H), 3.85-3.70 (m, 1H), 2.80-2.69 (m, 2H), 2.64 (s, 3H), 2.36 (s, 3H), 1.94 (td, J=11.8, 2.5 Hz, 2H), 1.82 (d, J=12.4 Hz, 2H), 1.51 (qd, J=12.0, 3.8 Hz, 2H). LCMS^{s} m/z=307 [M+H]⁺.

### Example 513: (R)-2-(7-methoxy-5-methyl-4-(methylamino)-2-((1-methylpyrrolidin-3-yl)amino)pyrido[2,3-d]pyrimidin-6-yl)ethan-1-ol

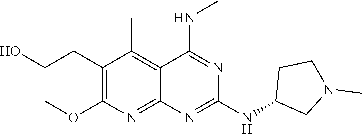 [SMILES: CNc1nc(N[C@@H]2CCN(C)C2)nc2nc(OC)c(CCO)c(C)c12]

### Preparation 197—(R)-6-chloro-7-methoxy-N⁴,5-dimethyl-N²-(1-methylpyrrolidin-3-yl)pyrido[2,3-d]pyrimidine-2,4-diamine

A solution of Example 616 (700 mg, 2.32 mmol) and NCS (737 mg, 4.63 mmol) in MeOH (2.00 mL) was stirred at 40° C. for 3 h. The resulting mixture was concentrated under reduced pressure. The residue was purified by Prep-HPLC^{BB} to afford the title compound as a yellow solid (400 mg, 51.3%). LCMS m/z=337 [M+H]⁺.

### Preparation 198—(R)-6-(2-ethoxyvinyl)-7-methoxy-N⁴,5-dimethyl-N²-(1-methylpyrrolidin-3-yl)pyrido[2,3-d]pyrimidine-2,4-diamine

To a stirred mixture of (R)-6-chloro-7-methoxy-N⁴,5-dimethyl-N²-(1-methylpyrrolidin-3-yl)pyrido[2,3-d]pyrimidine-2,4-diamine (380 mg, 1.13 mmol) and 2-[(E)-2-ethoxyethenyl]-4,4,5,5-tetramethyl-1,3,2-dioxaborolane (1.12 g, 5.64 mmol) in dioxane (5.00 mL) and H₂O (1.00 mL) were added K₂CO₃ (312 mg, 2.26 mmol) and XPhos Pd G3 (95.5 mg, 0.113 mmol), XPhos (107 mg, 0.226 mmol) at room temperature under nitrogen atmosphere. The resulting mixture was stirred under nitrogen atmosphere at 100° C. for 3 h. The mixture was allowed to cool down to room temperature. The resulting mixture was concentrated under reduced pressure. The residue was purified by Prep-HPLC^{BB} to afford the title compound as a yellow solid (200 mg, 47.6%). LCMS m/z=373 [M+H]⁺.

### Preparation 199—(R)-2-(7-methoxy-5-methyl-4-(methylamino)-2-((1-methylpyrrolidin-3-yl)amino)pyrido[2,3-d]pyrimidin-6-yl)acetaldehyde

A solution of (R)-6-(2-ethoxyvinyl)-7-methoxy-N⁴,5-dimethyl-N²-(1-methylpyrrolidin-3-yl)pyrido[2,3-d]pyrimidine-2,4-diamine (200 mg, 0.537 mmol) in TFA (0.60 mL) and DCM (3.00 ml) was stirred at room temperature for 2 h. The resulting mixture was concentrated under reduced pressure. This resulted in the title compound as a grey solid (200 mg). The crude product mixture was used in the next step directly without further purification. LCMS m/z=345 [M+H]⁺

### Preparation 200—(R)-2-(7-methoxy-5-methyl-4-(methylamino)-2-((1-methylpyrrolidin-3-yl)amino)pyrido[2,3-d]pyrimidin-6-yl)ethan-1-ol

To a stirred solution of (R)-2-(7-methoxy-5-methyl-4-(methylamino)-2-((1-methylpyrrolidin-3-yl)amino)pyrido[2,3-d]pyrimidin-6-yl)acetaldehyde (50.0 mg, 0.145 mmol) in MeOH (1.00 mL) was added NaBH₄ (54.9 mg, 1.45 mmol) at 0° C. The resulting mixture was stirred at room temperature for 2 h. The reaction was quenched with MeOH at room temperature. The resulting mixture was concentrated under reduced pressure. The residue was purified by Prep-HPLC^{BC} to afford the title compound as an off-white solid (10.0 mg, 17.7%, ee>99%). ¹H NMR (400 MHz, DMSO-d₆) δ 6.92-6.47 (m, 2H), 4.66 (s, 1H), 4.50-4.35 (m, 1H), 3.87 (s, 3H), 3.42 (t, J=7.5 Hz, 2H), 2.92 (d, J=4.2 Hz, 3H), 2.77 (t, J=9.5, 5.6 Hz, 3H), 2.60 (s, 3H), 2.56-2.52 (m, 1H), 2.47-2.39 (m, 1H), 2.31 (dd, J=9.2, 5.4 Hz, 1H), 2.23 (s, 3H), 2.20-2.09 (m, 1H), 1.73-1.63 (m, 1H). LCMS^{AU} m/z=347 [M+H]⁺.

### Example 514: (R)—N⁴,5-dimethyl-N²-(1-methylpyrrolidin-3-yl)-6,7-dihydrofuro[3′,2′:5,6]pyrido[2,3-d]pyrimidine-2,4-diamine

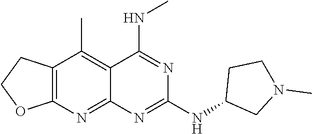 [SMILES: CNc1nc(N[C@@H]2CCN(C)C2)nc2nc3c(c(C)c12)CCO3]

A solution of Example 513 (50.0 mg, 0.144 mmol) in conc. HCl (1.50 mL, 12.0 M) was stirred at 100° C. overnight. The mixture was allowed to cool down to room temperature. The resulting mixture was concentrated under reduced pressure. The mixture was neutralized to pH 7 with NH₃—H₂O. The crude product was purified by Prep-HPLC^{A}N to afford the title compound as a white solid (7.40 mg, 16.3%, ee>99%). ¹H NMR (400 MHz, DMSO-d₆) δ 7.23-6.40 (m, 2H), 4.56 (t, J=8.3 Hz, 2H), 4.48-4.38 (m, 1H), 3.16 (t, J=8.3 Hz, 2H), 2.93-2.92 (m, 4H), 2.76-2.66 (m, 1H), 2.63-2.54 (m, 4H), 2.34 (s, 3H), 2.22-2.13 (m, 1H), 1.84-1.68 (m, 1H), 1.55-1.33 (m, 1H). LCMS^{W} m/z=315 [M+H]⁺.

### Example 515: (R)—N⁴,5,7-trimethyl-N²-(1-methylpyrrolidin-3-yl)-1,8-naphthyridine-2,4-diamine

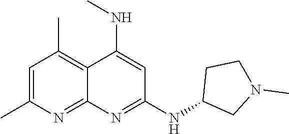

To a stirred mixture of 2-chloro-N,5,7-trimethyl-1,8-naphthyridin-4-amine (300 mg, 1.35 mmol) (Example 256, Preparation 155) and (3R)-1-methylpyrrolidin-3-amine (163 mg, 1.62 mmol) in dioxane (6.00 mL) was added Cs₂CO₃ (882 mg, 2.71 mmol), Pd(OAc)₂ (30.4 mg, 0.135 mmol) and xantphos (78.3 mg, 0.135 mmol). The mixture was stirred under nitrogen atmosphere at 100° C. for 1 h. The mixture was allowed to cool down to room temperature and concentrated under reduced pressure. The residue was purified by Prep-HPLC^{C} to afford the title compound (105.1 mg, 26.8%, ee>99%) as an off-white solid. ¹H NMR (400 MHz, DMSO-d₆) δ 6.73 (d, J=7.2 Hz, 1H), 6.63 (s, 1H), 5.81 (q, J=4.9 Hz, 1H), 5.67 (s, 1H), 4.58-4.41 (m, 1H), 2.78-2.71 (m, 4H), 2.69 (s, 3H), 2.63-2.54 (m, 1H), 2.41-2.31 (m, 5H), 2.25 (s, 3H), 2.23-2.14 (m, 1H), 1.69-1.52 (m, 1H). LCMS^{W} m/z=286 [M+H]⁺.

### Example 516: N²-((3S,4R)-4-methoxypiperidin-3-yl)-N⁴,5,7-trimethylpyrido[2,3-d]pyrimidine-2,4-diamine

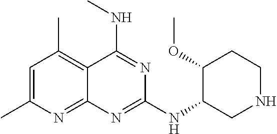 [SMILES: CNc1nc(N[C@H]2CNCC[C@H]2OC)nc2nc(C)cc(C)c12]

### Preparation 201—tert-butyl (3S,4R)-3-(1,3-dioxoisoindolin-2-yl)-4-hydroxypiperidine-1-carboxylate

To a stirred mixture of tert-butyl (3S,4R)-3-amino-4-hydroxypiperidine-1-carboxylate (800 mg, 3.69 mmol) and DIEA (1.93 mL, 11.0 mmol) in toluene (8.00 mL) at room temperature was added phthalic anhydride (712 mg, 4.80 mmol). The resulting mixture was stirred at 110° C. overnight. The mixture was allowed to cool down to room temperature and concentrated under reduced pressure. The residue was diluted with water (20.0 mL) and extracted with EtOAc (3×20 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (1:1) to afford the title compound as a white solid (750 mg, 58.5%). LCMS m/z=347 [M+H]⁺.

### Preparation 202—tert-butyl (3S,4R)-3-(1,3-dioxoisoindolin-2-yl)-4-methoxypiperidine-1-carboxylate

To a stirred mixture of tert-butyl (3S,4R)-3-(1,3-dioxoisoindol-2-yl)-4-hydroxypiperidine-1-carboxylate (500 mg, 1.44 mmol) and 4 Å MS (1.00 g) in DMF (10.0 mL) at room temperature were added potassium tert-butoxide (323 mg, 2.88 mmol) and Mel (1.23 g, 8.65 mmol). The resulting mixture was stirred at room temperature for 1 h. The reaction was diluted with EtOAc (100 mL) and washed with water (2×80 mL). The organic layer was dried over anhydrous Na₂SO₄. After filtration, the filtrate was concentrated under reduced pressure. The residue was purified by silica gel column chromatography, eluted with PE/EA (1:1) to afford the title compound as a white solid (320 mg, 61.3%). LCMS m/z=361 [M+H]⁺.

### Preparation 203—tert-butyl (3S,4R)-3-amino-4-methoxypiperidine-1-carboxylate

A mixture of tert-butyl (3S,4R)-3-(1,3-dioxoisoindol-2-yl)-4-methoxypiperidine-1-carboxylate (270 mg, 0.749 mmol) and N₂H₄·H₂O (0.50 mL, 80% wt) in EtOH (2.50 mL) was stirred at 70° C. for 1 h. The reaction was allowed to cool down to room temperature. The resulting mixture was filtered; the filter cake was washed with 1,4-dioxane (3×5 mL). The filtrate was concentrated under reduced pressure to afford the title compound as a colourless oil (170 mg, crude). LCMS m/z=231 [M+H]⁺.

### Preparation 204—tert-butyl (3S,4R)-3-((5,7-dimethyl-4-(methylamino)pyrido[2,3-d]pyrimidin-2-yl)amino)-4-methoxypiperidine-1-carboxylate

To a stirred solution of tert-butyl (3S,4R)-3-amino-4-methoxypiperidine-1-carboxylate (150 mg, 0.651 mmol) in dioxane (3.00 mL) was added 2-chloro-N,5,7-trimethylpyrido[2,3-d]pyrimidin-4-amine (174 mg, 0.781 mmol) (Example 227, Preparation 108) at room temperature. The resulting mixture was stirred at 100° C. overnight. The resulting mixture was allowed to cool down to room temperature, then it was concentrated under reduced pressure. The residue was purified by silica gel column chromatography, eluted with CH₂Cl₂/MeOH (12:1) to afford the title compound as a yellow solid (220 mg, 81.2%). LCMS m/z=417 [M+H]⁺.

### Preparation 205—N²-((3S,4R)-4-methoxypiperidin-3-yl)-N⁴,5,7-trimethylpyrido[2,3-d]pyrimidine-2,4-diamine

A mixture of tert-butyl (3S,4R)-3-([5,7-dimethyl-4-(methylamino)pyrido[2,3-d]pyrimidin-2-yl]amino-4-methoxypiperidine-1-carboxylate (200 mg, 0.480 mmol) and HCl (gas) in 1,4-dioxane (4.00 mL, 4.0 M) was stirred at room temperature for 1 h. The resulting mixture was concentrated under reduced pressure. The crude product was purified by Prep-HPLC^{C} to afford the title compound as a white solid (100 mg, 65.8%, ee>99%). ¹H NMR (400 MHz, DMSO-d₆) δ 6.76 (s, 1H), 6.70 (s, 1H), 5.80 (s, 1H), 4.25 (s, 1H), 3.55-3.46 (m, 1H), 3.28 (s, 3H), 2.95 (d, J=4.0 Hz, 3H), 2.82-2.68 (m, 3H), 2.65 (s, 3H), 2.59-2.52 (m, 1H), 2.37 (s, 3H), 1.87-1.72 (m, 1H), 1.60-1.48 (m, 1H). LCMS^{AK} m/z=317 [M+H]⁺.

### Example 517: N²-(-7-azabicyclo[2.2.1]heptan-2-yl)-N⁴,5,7-trimethylpyrido[2,3-d]pyrimidine-2,4-diamine

### Example 518: N²-(-7-azabicyclo[2.2.1]heptan-2-yl)-N⁴,5,7-trimethylpyrido[2,3-d]pyrimidine-2,4-diamine

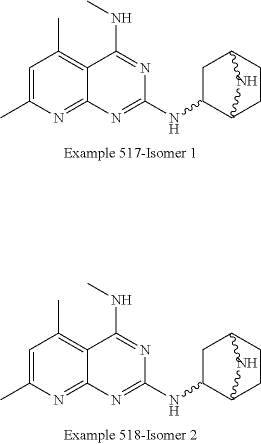

### Preparation 206—tert-butyl-endo-2-((5,7-dimethyl-4-(methylamino)pyrido[2,3-d]pyrimidin-2-yl)amino)-7-azabicyclo[2.2.1]heptane-7-carboxylate

To a stirred solution of 2-chloro-N,5,7-trimethylpyrido[2,3-d]pyrimidin-4-amine (400 mg, 1.79 mmol) in DMSO (4.00 mL) was added tert-butyl (1R,2R,4S)-rel-2-amino-7-azabicyclo[2.2.1]heptane-7-carboxylate (457 mg, 2.15 mmol). The resulting mixture was stirred at 120° C. overnight. The mixture was allowed to cool down to room temperature. The residue was purified by Prep-HPLC^{L} to afford the title compound as a yellow solid (550 mg, 76.8%). LCMS m/z=399 [M+H]⁺.

### Preparation 207—N²-[(1S,2S,4R)-7-azabicyclo[2.2.1]heptan-2-yl]-N⁴,5,7-trimethylpyrido[2,3-d]pyrimidine-2,4-diamine and N²-[(1R,2R,4S)-7-azabicyclo[2.2.1]heptan-2-yl]-N⁴,5,7-trimethylpyrido[2,3-d]pyrimidine-2,4-diamine

A mixture of tert-butyl-endo-2-((5,7-dimethyl-4-(methylamino)pyrido[2,3-d]pyrimidin-2-yl)amino)-7-azabicyclo[2.2.1]heptane-7-carboxylate (500 mg, 1.26 mmol) and HCl (gas) in 1,4-dioxane (5.00 mL) was stirred at room temperature for 1 h. The resulting mixture was concentrated under reduced pressure. The residue was purified by Prep-HPLC^{L} to afford the title compound (400 mg). The product was further purified by Chiral-HPLC^{A} to afford Example 517 (200 mg, ee>99%, Rt: 21.3 min) as a white solid and Example 518 (190 mg, ee: 93.8%, Rt: 24.6 min) as a white solid. The stereochemistry of the isolated isomers have not been unambiguously confirmed

Example 517: ¹H NMR (400 MHz, Methanol-d₄) δ 6.77 (s, 1H), 4.39-4.27 (m, 1H), 4.14-3.88 (m, 1H), 3.61 (t, J=4.9 Hz, 1H), 3.07 (s, 3H), 2.70 (s, 3H), 2.46 (s, 3H), 2.26-2.13 (m, 1H), 2.01-1.86 (m, 1H), 1.75-1.62 (m, 1H), 1.61-1.42 (m, 2H), 1.26-1.13 (m, 1H). LCMS^{AK} m/z=299 [M+H]⁺.

Example 518: ¹H NMR (400 MHz, Methanol-d₄) δ 6.77 (s, 1H), 4.42-4.29 (m, 1H), 4.22-3.87 (m, 1H), 3.71-3.55 (m, 1H), 3.07 (s, 3H), 2.71 (s, 3H), 2.46 (s, 3H), 2.26-2.17 (m, 1H), 2.00-1.89 (m, 1H), 1.77-1.63 (m, 1H), 1.62-1.44 (m, 2H), 1.30-1.16 (m, 1H). LCMS^{AK} m/z=299 [M+H]⁺.

### Example 519: N²-((3S,4R)-1-cyclopropyl-4-fluoropyrrolidin-3-yl)-N⁴,5,7-trimethylpyrido[2,3-d]pyrimidine-2,4-diamine

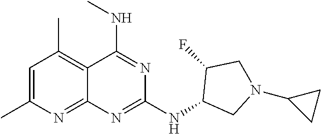 [SMILES: CNc1nc(N[C@H]2CN(C3CC3)C[C@H]2F)nc2nc(C)cc(C)c12]

To a solution of Example 335 (100 mg, 0.34 mmol) in MeOH (2.00 mL) and AcOH (0.120 mL) at room temperature was added (1-ethoxycyclopropoxy)trimethylsilane (90.1 mg, 0.52 mmol). The resulting mixture was stirred at 60° C. for 1 h. To the above mixture was added NaBH₃CN (108 mg, 1.72 mmol) at 60° C. The resulting mixture was stirred at 60° C. overnight. The reaction was quenched with sat. NH₄Cl (aq.) at room temperature. The resulting mixture was concentrated under reduced pressure. The residue was purified by Prep-HPLC^{BA} to afford the title compound as a white solid (35.4 mg, 31.1%, ee>95%). ¹H NMR (400 MHz, DMSO-d₆) δ 7.01-6.76 (m, 1H), 6.73 (s, 1H), 6.64-6.41 (m, 1H), 5.14 (dtd, J=55.9, 4.7, 1.6 Hz, 1H), 4.57-4.35 (m, 1H), 3.28-3.18 (m, 1H), 3.04 (t, J=8.3 Hz, 1H), 2.95 (d, J=4.4 Hz, 3H), 2.91-2.72 (m, 2H), 2.66 (s, 3H), 2.38 (s, 3H), 1.90-1.78 (m, 1H), 0.47-0.27 (m, 4H). LCMS^{A}N m/z=331 [M+H]⁺.

### Example 520: (3S,4S)-1-methyl-4-((5,6,7-trimethyl-4-(methylamino)pyrido[2,3-d]pyrimidin-2-yl)amino)pyrrolidin-3-ol

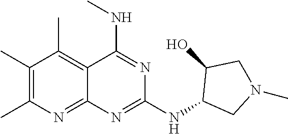

To a mixture of Example 629 (100 mg, 0.290 mmol) and methylboronic acid (35.0 mg, 0.590 mmol) in dioxane (5.00 mL) were added SPhos Pd G3 (23.0 mg, 0.03 mmol), SPhos (12.0 mg, 0.03 mmol) and K₂CO₃ (82.0 mg, 0.590 mmol). The resulting mixture was stirred under nitrogen atmosphere at 80° C. overnight. The mixture was allowed to cool down to room temperature. The resulting mixture was concentrated under reduced pressure. The residue was purified by Prep-HPLC^{C} to afford the title compound as a white solid (25.6 mg, 26.9%). ¹H NMR (400 MHz, DMSO-d₆) δ 6.97-6.80 (m, 2H), 6.14 (s, 1H), 4.05-3.83 (m, 2H), 2.94 (d, J=4.4 Hz, 3H), 2.90-2.82 (m, 1H), 2.71 (dd, J=9.5, 6.8 Hz, 1H), 2.58 (s, 3H), 2.46-2.37 (m, 5H), 2.21 (s, 3H), 2.19 (s, 3H). LCMS^{AK} m/z=317 [M+H]⁺.

### Example 521: 7-cyclobutyl-5-methyl-N²-(1-methylpiperidin-4-yl)pyrido[2,3-d]pyrimidine-2,4-diamine

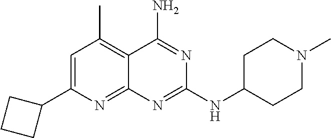 [SMILES: Cc1cc(C2CCC2)nc2nc(NC3CCN(C)CC3)nc(N)c12]

To a stirred solution of Example 2 (100 mg, 0.367 mmol) and potassium cyclobutyltrifluoroboranuide (119 mg, 0.734 mmol) in HOAc (0.75 mL) and H₂O (0.75 mL) at room temperature were added manganese triacetate dihydrate (197 mg, 0.734 mmol) and conc. H₂SO₄ (3.70 mg, 0.037 mmol). The resulting mixture was stirred at 60° C. overnight. The mixture was allowed to cool down to room temperature and concentrated under reduced pressure. The crude product was purified by Prep-HPLC^{AV} to afford the title compound (17.9 mg, 14.7%) as a white solid. ¹H NMR (400 MHz, DMSO-d₆) δ 6.91-6.51 (m, 3H), 6.47-6.24 (m, 1H), 3.91-3.72 (m, 1H), 3.63-3.46 (m, 1H), 2.82-2.70 (m, 2H), 2.65 (s, 3H), 2.36-2.20 (m, 4H), 2.16 (s, 3H), 2.07-1.91 (m, 3H), 1.88-1.74 (m, 3H), 1.60-1.42 (m, 2H). LCMS^{W}m/z=327 [M+H]⁺.

### Example 522: N²-(3-(dimethylamino)propyl)-N⁴,3,5,7-tetramethyl-1,8-naphthyridine-2,4-diamine

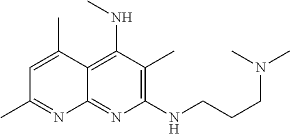

### Preparation 208—N¹-(4-chloro-3,5,7-trimethyl-1,8-naphthyridin-2-yl)-N³, N³-dimethylpropane-1,3-diamine

To a stirred solution of 2,4-dichloro-3,5,7-trimethyl-1,8-naphthyridine (500 mg, 2.07 mmol) (Example 257, Preparation 159) and N¹, N¹-dimethylpropane-1,3-diamine (423 mg, 4.14 mmol) in dioxane (5.00 mL) was added CsF (629 mg, 4.14 mmol) at room temperature. The resulting mixture was stirred at 100° C. overnight. The resulting mixture was concentrated under reduced pressure. The residue was purified by prep-HPLC^{C} to afford the title compound as yellow solid (410 mg, 64.4%). LCMS m/z=307 [M+H]⁺.

### Preparation 209—N²-(3-(dimethylamino)propyl)-N⁴,3,5,7-tetramethyl-1,8-naphthyridine-2,4-diamine

To a stirred solution of N¹-(4-chloro-3,5,7-trimethyl-1,8-naphthyridin-2-yl)-N³, N³-dimethylpropane-1,3-diamine (160 mg, 0.521 mmol) and methylamine hydrochloride (70.0 mg, 1.04 mmol) in dioxane (4.00 mL) was added Pd₂(dba)₃ (96.0 mg, 0.104 mmol), Xantphos (60.0 mg, 0.104 mmol) and t-BuOK (117 mg, 1.04 mmol). The resulting mixture was stirred under nitrogen atmosphere at 110° C. overnight. The resulting mixture was concentrated under reduced pressure. The residue was purified by prep-HPLC^{AQ} to afford the title compound as yellow oil (3.60 mg, 2.29%). ¹H NMR (400 MHz, DMSO-d₆) 1H NMR (400 MHz, Methanol-d₄) δ 6.83 (s, 1H), 3.64 (t, J=6.5 Hz, 2H), 2.84 (s, 3H), 2.79 (s, 3H), 2.64 (t, J=7.1 Hz, 2H), 2.50 (s, 3H), 2.44 (s, 6H), 2.15 (s, 3H), 1.93 (p, J=6.9 Hz, 2H). LCMS^{W} m/z=302 [M+H]⁺.

### Example 523: N²-((3S,4R)-4-fluoro-1-isopropylpyrrolidin-3-yl)-N⁴,5,7-trimethylpyrido[2,3-d]pyrimidine-2,4-diamine

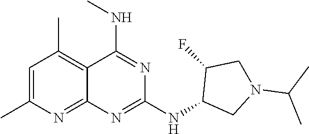 [SMILES: CNc1nc(N[C@H]2CN(C(C)C)C[C@H]2F)nc2nc(C)cc(C)c12]

To a mixture of Example 335 (100 mg, 0.344 mmol) and acetone (0.40 ml) in DCE (2.00 mL) at room temperature was added STAB (146 mg, 0.690 mmol) and AcOH (53.8 mg, 0.890 mmol). The resulting mixture was stirred at room temperature for 1 h. The reaction was quenched with water at room temperature and then it was neutralized to pH 7 with Et₃N. The resulting mixture was concentrated under reduced pressure. The residue was purified by Prep-HPLC^{AU} to afford the title compound as a white solid (34.5 mg, 30.1%, ee>95%). ¹H NMR (400 MHz, DMSO-d₆) δ 7.01-6.63 (m, 2H), 6.61-6.30 (m, 1H), 5.14 (dtd, J=56.2, 4.8, 1.5 Hz, 1H), 4.55-4.39 (m, 1H), 3.26-3.09 (m, 1H), 3.06-2.88 (m, 4H), 2.81-2.64 (m, 4H), 2.60 (t, J=9.1 Hz, 1H), 2.57-2.52 (m, 1H), 2.38 (s, 3H), 1.02 (t, J=6.2 Hz, 6H). ¹⁹F NMR (377 MHz, DMSO-d₆) 5-187.86. LCMS^{AN} m/z=333 [M+H]⁺.

### Example 524: (R)—N⁴,N⁷,5-trimethyl-N²-(1-methylpyrrolidin-3-yl)pyrido[2,3-d]pyrimidine-2,4,7-triamine

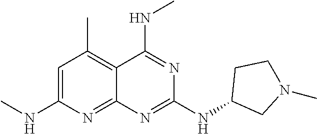

### Preparation 210—2,6-dichloro-4-methylnicotinamide

A solution of 2,6-dichloro-4-methylpyridine-3-carbonitrile (10.0 g, 53.5 mmol) in con. H₂SO₄ (50.0 mL) was stirred at 80° C. for 2 h. The mixture was allowed to cool down to room temperature. The resulting mixture was diluted with water (400 mL). The precipitated solids were collected by filtration and washed with water (3×50 mL). This resulted in the title compound (10.1 g, 92.1%) as an off-white solid. LCMS m/z=205, 207 [M+H]⁺.

### Preparation 211—2-amino-6-chloro-4-methylnicotinamide

A solution of 2,6-dichloro-4-methylpyridine-3-carboxamide (4.00 g, 19.5 mmol) and NH₃—H₂O (80.0 mL) in dioxane (260 mL) was stirred at 160° C. overnight. The resulting mixture was concentrated under reduced pressure. The residue was purified by silica gel column chromatography, eluted with PE/EA (1:4) to afford to the title compound (3.60 g, 64.6%) as an off-white solid. LCMS m/z=186 [M+H]⁺.

### Preparation 212—7-chloro-5-methylpyrido[2,3-d]pyrimidine-2,4(1H,3H)-dione

To a stirred solution of 2-amino-6-chloro-4-methylpyridine-3-carboxamide (2.00 g, 10.8 mmol) in Toluene (30.0 mL) at room temperature was added (COCl)₂ (6.84 g, 53.9 mmol). The resulting mixture was stirred at 110° C. overnight. The mixture was allowed to cool down to room temperature. The reaction was quenched by the addition of NaHCO₃ (aq.) at room temperature. The resulting mixture was extracted with EtOAc (3×300 mL). The combined organic layers were washed with brine (2×200 mL), dried over anhydrous Na₂SO₄. After filtration, the filtrate was concentrated under reduced pressure. This resulted in to the title compound (1.65 g, 72.4%) as a yellow green solid. LCMS m/z=212 [M+H]⁺.

### Preparation 213—5-methyl-7-(methylamino)pyrido[2,3-d]pyrimidine-2,4-diol

To a stirred solution of 7-chloro-5-methyl-1H,3H-pyrido[2,3-d]pyrimidine-2,4-dione (500 mg, 2.36 mmol) and CH₃NH₂—HCl (478 mg, 7.09 mmol) in DMSO (7.50 mL) at room temperature was added DIEA (916 mg, 7.09 mmol). The resulting mixture was stirred at 100° C. overnight. The mixture was allowed to cool down to room temperature. The precipitated solids were collected by filtration and washed with EtOAc (2×10 mL). This resulted in the title compound (500 mg, crude) as an off-white solid. LCMS m/z=207 [M+H]⁺.

### Preparation 214—2,4-dichloro-N,5-dimethylpyrido[2,3-d]pyrimidin-7-amine

To a stirred solution of 5-methyl-7-(methylamino)pyrido[2,3-d]pyrimidine-2,4-diol (500 mg, 2.42 mmol) in POCl₃ (7.50 mL) at room temperature was added DIEA (1.58 g, 12.1 mmol) dropwise. The resulting mixture was stirred at 100° C. for 2 h. The mixture was allowed to cool down to room temperature. The resulting mixture was concentrated under reduced pressure, then it was quenched by the addition of NaHCO₃ (aq.) (100 mL) at 0° C. The precipitated solids were collected by filtration and washed with water (2×10 mL). This resulted in the title compound (300 mg, crude) as an off-white solid. LCMS m/z=243, 245 [M+H]⁺.

### Preparation 215—2-chloro-N⁴,N⁷,5-trimethylpyrido[2,3-d]pyrimidine-4,7-diamine

To a stirred solution of 2,4-dichloro-N,5-dimethylpyrido[2,3-d]pyrimidin-7-amine (300 mg, 1.23 mmol) in THF (4.50 mL) at room temperature was added CH₃NH₂ (256 mg, 2.47 mmol, 30% wt in water). The resulting mixture was stirred at room temperature for 2 h. The reaction was quenched with Water at room temperature. The precipitated solids were collected by filtration and washed with water (3×5 mL). This resulted in the title compound (180 mg, 61.4%) as an off-white solid. LCMS m/z=238 [M+H]⁺.

### Preparation 216—(R)—N⁴,N⁷,5-trimethyl-N²-(1-methylpyrrolidin-3-yl)pyrido[2,3-d]pyrimidine-2,4,7-triamine

To a stirred solution of 2-chloro-N⁴,5,N′-trimethylpyrido[2,3-d]pyrimidine-4,7-diamine (150 mg, 0.631 mmol) in DMSO (3.00 mL) was added (3R)-1-methylpyrrolidin-3-amine (126 mg, 1.26 mmol). The resulting mixture was stirred at 100° C. overnight. The mixture was allowed to cool down to room temperature. The crude product was purified by Prep-HPLC^{AQ} to afford the title compound (40.0 mg, 21.0%, ee>99%) as a white solid. ¹H NMR (400 MHz, DMSO-d₆) δ 6.79-6.75 (m, 1H), 6.29 (br, 2H), 5.96 (s, 1H), 4.47-4.36 (m, 1H), 2.88 (d, J=4.3 Hz, 3H), 2.78 (d, J=4.7 Hz, 3H), 2.76-2.67 (m, 1H), 2.56-2.50 (m, 4H), 2.44-2.37 (m, 1H), 2.33-2.27 (m, 1H), 2.23 (s, 3H), 2.19-2.06 (m, 1H), 1.72-1.59 (m, 1H). LCMS^{W} m/z=302 [M+H]⁺.

### Example 525: (R)-5-methoxy-7-methyl-N²-(1-methylpyrrolidin-3-yl)pyrido[2,3-d]pyrimidine-2,4-diamine

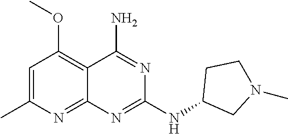

### Preparation 217—ethyl 2-chloro-4-methoxy-6-methylnicotinate

A mixture of ethyl 2,4-dichloro-6-methylnicotinate (12.0 g, 51.5 mmol) and MeONa (11.1 g, 206 mmol) in MeOH (50.0 mL) was stirred at 80° C. for 2 h. The mixture was allowed to cool down to room temperature, then it was quenched with water (250 mL). The resulting mixture was extracted with EtOAc (3×250 mL). The combined organic layers were washed with brine (250 mL), dried over anhydrous Na₂SO₄. After filtration, the filtrate was concentrated under reduced pressure. The residue was purified by Prep-HPLC^{AD} to afford the title compound (5.80 g, 48.9%) as a white solid. LCMS m/z=230 [M+H]⁺.

### Preparation 218—ethyl 4-methoxy-2-((4-methoxybenzyl)amino)-6-methylnicotinate

A solution of ethyl 2-chloro-4-methoxy-6-methylnicotinate (5.40 g, 23.5 mmol) in (4-methoxyphenyl)methanamine (50.0 mL) was stirred at 140° C. for 1 h. The mixture was allowed to cool down to room temperature and diluted with water (250 mL). The resulting mixture was extracted with EtOAc (3×250 mL). The combined organic layers were washed with brine (250 mL), dried over anhydrous Na₂SO₄. After filtration, the filtrate was concentrated under reduced pressure. The residue was purified by silica gel column chromatography, eluted with PE/EA (10:1) to afford the title compound (5.70 g, 73.4%) as a colourless oil. LCMS m/z=331 [M+H]⁺.

### Preparation 219—ethyl 2-amino-4-methoxy-6-methylnicotinate

A mixture of ethyl 4-methoxy-2-((4-methoxybenzyl)amino)-6-methylnicotinate (5.70 g, 17.3 mmol) in TFA (25.0 mL) and DCM (25.0 mL) was stirred at room temperature for 1 h. The resulting mixture was concentrated under reduced pressure and dissolved in water (100 mL). The residue was basified to pH 8 with NaOH (1N) at 0° C. The resulting mixture was extracted with EtOAc (3×100 mL). The combined organic layers were washed with brine (100 mL), dried over anhydrous Na₂SO₄. After filtration, the filtrate was concentrated under reduced pressure. The residue was purified by trituration with PE (50.0 mL). This resulted in the title compound (4.70 g, crude) as a white solid. LCMS m/z=211 [M+H]⁺.

### Preparation 220—ethyl 4-methoxy-6-methyl-2-(3-(2,2,2-trichloroacetyl)ureido)nicotinate

To a stirred solution of ethyl 2-amino-4-methoxy-6-methylnicotinate (4.60 g, 21.9 mmol) in THF (100 mL) was added 2,2,2-trichloroacetyl isocyanate (4.12 g, 21.9 mmol) dropwise at 0° C. The resulting mixture was stirred at 0° C. for 1 h. The resulting mixture was concentrated under reduced pressure and purified by trituration with PE (100 mL). This resulted in the title compound (5.60 g, 64.2%) as a white solid. LCMS m/z=398/400 [M+H]⁺.

### Preparation 221—5-methoxy-7-methylpyrido[2,3-d]pyrimidine-2,4(1H,3H)-dione

To a stirred mixture of ethyl 4-methoxy-6-methyl-2-(3-(2,2,2-trichloroacetyl)ureido)nicotinate (5.60 g, 14.0 mmol) in EtOH (100 mL) was added NH₃(g) in MeOH (22.4 mL) dropwise at room temperature. The resulting mixture was stirred at 40° C. for 3 h. The mixture was allowed to cool down to room temperature. The precipitated solids were collected by filtration and washed with EtOAc (3×50 mL). This resulted in the title compound (2.70 g, 92.8%) as a white solid. LCMS m/z=208 [M+H]⁺.

### Preparation 222—2,4-dichloro-5-methoxy-7-methylpyrido[2,3-d]pyrimidine

To a stirred mixture of 5-methoxy-7-methylpyrido[2,3-d]pyrimidine-2,4(1H,3H)-dione (1.00 g, 4.83 mmol) in DCM (10.0 mL) was added POCl₃ (2.25 mL, 24.1 mmol) and N,N-diethylaniline (3.60 g, 24.1 mmol) dropwise at room temperature. The resulting mixture was stirred at 80° C. for 2 h. The mixture was allowed to cool down to room temperature and concentrated under reduced pressure. The mixture was quenched by the addition of sat. NaHCO₃ (aq.) (200 mL) at 0° C. The resulting mixture was extracted with CH₂Cl₂ (3×200 mL). The combined organic layers were washed with brine (200 mL), dried over anhydrous Na₂SO₄. After filtration, the filtrate was concentrated under reduced pressure. The residue was purified by silica gel column chromatography, eluted with PE/EA (1:5) to afford the title compound (300 mg, 25.5%) as a yellow solid. LCMS m/z=244/246 [M+H]⁺.

### Preparation 223—2-chloro-5-methoxy-7-methylpyrido[2,3-d]pyrimidin-4-amine

A solution of 2,4-dichloro-5-methoxy-7-methylpyrido[2,3-d]pyrimidine (170 mg, 0.696 mmol) in NH₃—H₂O (3.00 mL) was stirred at room temperature for 1 h. The precipitated solids were collected by filtration and washed with water (3×2 mL). This afford the title compound (120 mg, 76.7%) as an off-white solid. LCMS m/z=225 [M+H]⁺.

### Preparation 224—(R)-5-methoxy-7-methyl-N²-(1-methylpyrrolidin-3-yi)pyrido[2,3-d]pyrimidine-2,4-diamine

A mixture of 2-chloro-5-methoxy-7-methylpyrido[2,3-d]pyrimidin-4-amine (110 mg, 0.490 mmol), CsF (149 mg, 0.980 mmol) and (R)-1-methylpyrrolidin-3-amine (58.9 mg, 0.588 mmol) in dioxane (2.00 mL) was stirred under nitrogen atmosphere at 100° C. overnight. The mixture was allowed to cool down to room temperature and concentrated under reduced pressure. The residue was purified by Prep-HPLC^{C} to afford the crude product (50.0 mg). The crude product was further purified by Prep-HPLC^{AP} to afford the title compound (19.6 mg, 13.9%) as a white solid. H NMR (400 MHz, DMSO-d₆) δ 7.19 (s, 2H), 6.77-6.35 (m, 2H), 4.46-4.35 (1H), 3.95 (s, 3H), 2.75 (t, J=8.2 Hz, 1H), 2.57-2.51 (m, 1H), 2.46-2.36 (m, 4H), 2.35-2.27 (m, 1H), 2.23 (s, 3H), 2.19-2.05 (m, 1H), 1.72-1.61 (m, 1H). LCMS^{AF} m/z=289 [M+H]⁺.

### Example 526: 4-((4-((4-amino-5-methyl-7,8-dihydro-6H-cyclopenta[5,6]pyrido[2,3-d]pyrimidin-2-yl)amino)piperidin-1-yl)methyl)-3-hydroxybenzonitrile

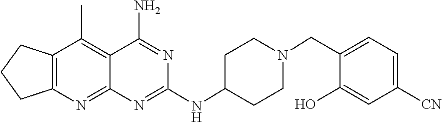 [SMILES: Cc1c2c(nc3nc(NC4CCN(Cc5ccc(C#N)cc5O)CC4)nc(N)c13)CCC2]

### Preparation 225—ethyl 3-amino-1-methyl-6,7-dihydro-5H-cyclopenta[c]pyridine-4-carboxylate and ethyl 2-amino-4-methyl-6,7-dihydro-5H-cyclopenta[b]pyridine-3-carboxylate

To a stirred mixture of ethyl 2-carbamimidoylacetate hydrochloride (66.0 g, 396 mmol) and piperidine (101 g, 1190 mmol) in EtOH (1.00 L) at room temperature was added acetylcyclopentanone (50.0 g, 396 mmol) dropwise. The resulting mixture was stirred at room temperature for 2 h. The resulting mixture was concentrated under reduced pressure. The residue was diluted with PE (100 mL). The precipitated solids were collected by filtration and purified by silica gel column with ethyl acetate/petroleum ether (3:1) to afford ethyl 3-amino-1-methyl-6,7-dihydro-5H-cyclopenta[c]pyridine-4-carboxylate (35.0 g, 40.1%) as white solid. The filtrate was concentrated under reduced pressure. The residue was purified by Prep-H PLC^{A}W to afford ethyl 2-amino-4-methyl-6,7-dihydro-5H-cyclopenta[b]pyridine-3-carboxylate (21.0 g, 24.0%) as off-white solid. LCMS m/z=221 [M+H]⁺.

### Preparation 226—ethyl 4-methyl-2-(3-(2,2,2-trichloroacetyl)ureido)-6,7-dihydro-5H-cyclopenta[b]pyridine-3-carboxylate

To a stirred solution of ethyl 2-amino-4-methyl-6,7-dihydro-5H-cyclopenta[b]pyridine-3-carboxylate (21.0 g, 95.0 mmol) in THE (210 mL) was added trichloroethanecarbonyl isocyanate (26.8 g, 142 mmol) dropwise at 0° C. The resulting mixture was stirred at room temperature for 1 h. The resulting mixture was concentrated under reduced pressure. The residue was purified by silica gel column with ethyl acetate/petroleum ether (3:1) to afford the title compound as white solid (38.0 g, 98.0%). LCMS m/z=408 [M+H]⁺.

### Preparation 227—5-methyl-7,8-dihydro-6H-cyclopenta[5,6]pyrido[2,3-d]pyrimidine-2,4-diol

Into a pressure tank reactor were added ethyl 1-methyl-3-(3-(2,2,2-trichloroacetyl)ureido)-6,7-dihydro-5H-cyclopenta[c]pyridine-4-carboxylate (38.0 g, 93.1 mmol) and 7.0 M NH₃ in MeOH (70.0 mL) in EtOH (460 mL) at room temperature. The resulting mixture was stirred at 80° C. overnight. The mixture was allowed to cool down to 0° C. The precipitated solids were collected by filtration and washed with EtOH (3×20 mL). This resulted in the title compound (19.2 g, 95.0%) as white solid. LCMS m/z=218 [M+H]⁺.

### Preparation 228—2,4-dichloro-5-methyl-7,8-dihydro-6H-cyclopenta[5,6]pyrido[2,3-d]pyrimidine

To a stirred mixture of 5-methyl-7,8-dihydro-6H-cyclopenta[5,6]pyrido[2,3-d]pyrimidine-2,4-diol (22.0 g, 101 mmol) and DIEA (65.5 g, 506 mmol) in MeCN (400 mL) at 0° C. was added POCl₃ (155 g, 1010 mmol) dropwise. The resulting mixture was stirred at 60° C. for 1 h. The mixture was allowed to cool down to room temperature. To the above mixture was added HCl (g) (4.0 M in 1,4-dioxane) (220 mL, 880 mmol) dropwise over 30 min at room temperature. The resulting mixture was stirred at 60° C. for additional 1 h. The resulting mixture was cooled to room temperature and concentrated under reduced pressure. The residue was diluted with DCM (3.0 L) and quenched by added to sat. NaHCO₃ (aq.) at 0° C. The resulting mixture was filtered, the filter cake was washed with DCM (3×1 L). The combined organic layers were washed with brine (3×1 L), dried over anhydrous Na₂SO₄. After filtration, the filtrate was concentrated under reduced pressure. This resulted in the title compound as brown solid (21.0 g, crude). LCMS m/z=254 [M+H]⁺.

### Preparation 229—2-chloro-5-methyl-7,8-dihydro-6H-cyclopenta[5,6]pyrido[2,3-d]pyrimidin-4-amine

To a stirred solution of 2,4-dichloro-5-methyl-7,8-dihydro-6H-cyclopenta[5,6]pyrido[2,3-d]pyrimidine (21.0 g, 82.6 mmol) in MeCN (1.00 L) at room temperature was added NH₃—H₂O (400 mL). The resulting mixture was stirred at 60° C. for 1 h. The mixture was allowed to cool down to room temperature. The resulting mixture was concentrated under reduced pressure. This resulted in the title compound as brown solid (23.0 g, crude). LCMS m/z=235 [M+H]⁺.

### Preparation 230—tert-butyl 4-((4-amino-5-methyl-7,8-dihydro-6H-cyclopenta[5,6]pyrido[2,3-d]pyrimidin-2-yl)amino)piperidine-1-carboxylate

To a stirred solution of 2-chloro-5-methyl-7,8-dihydro-6H-cyclopenta[5,6]pyrido[2,3-d]pyrimidin-4-amine (23.0 g, 98.3 mmol) in DMSO (460 mL) was added tert-butyl 4-aminopiperidine-1-carboxylate (55.0 g, 275 mmol) at room temperature. The resulting mixture was stirred at 120° C. for 8 h. The mixture was allowed to cool down to room temperature. The resulting mixture was diluted with water (1000 mL) and extracted with CH₂Cl₂ (3×500 mL). The combined organic layers were washed with brine (3×300 mL), dried over anhydrous Na₂SO₄. After filtration, the filtrate was concentrated under reduced pressure. The residue was purified by Prep-HPLC^{A}W to afford the title compound as an orange solid (6.39 g, 15.8% yield over 3 steps). LCMS m/z=399 [M+H]⁺.

### Preparation 231—5-methyl-N²-(piperidin-4-yl)-7,8-dihydro-6H-cyclopenta[5,6]pyrido[2,3-d]pyrimidine-2,4-diamine

A mixture of tert-butyl 4-((4-amino-5-methyl-7,8-dihydro-6H-cyclopenta[5,6]pyrido[2,3-d]pyrimidin-2-yl)amino)piperidine-1-carboxylate (3.90 g, 9.79 mmol) and 4 M HCl in 1,4-dioxane) (39.0 mL) was stirred at room temperature for 1 h. The resulting mixture was concentrated under reduced pressure to afford the title compound as a reddish brown solid (4.20 g, crude). LCMS m/z=299 [M+H]⁺.

### Preparation 232—4-((4-((4-amino-5-methyl-7,8-dihydro-6H-cyclopenta[5,6]pyrido[2,3-d]pyrimidin-2-yl)amino)piperidin-1-yl)methyl)-3-hydroxybenzonitrile

A solution of 5-methyl-N²-(piperidin-4-yl)-7,8-dihydro-6H-cyclopenta[5,6]pyrido[2,3-d]pyrimidine-2,4-diamine (150 mg, 0.503 mmol) and 4-formyl-3-hydroxybenzonitrile (148 mg, 1.01 mmol) in DCE (4.00 mL) was treated with Et₃N (102 mg, 1.01 mmol) at 40° C. for 2 h followed by the addition of STAB (426 mg, 2.01 mmol) at room temperature. The resulting mixture was stirred at 40° C. for 2 h. The mixture was allowed to cool down to room temperature and quenched with water (5.0 mL). The resulting mixture was concentrated under reduced pressure. The residue was purified by Prep-HPLCA to afford the title compound as an orange solid (59.4 mg, 27.5%). ¹H NMR (400 MHz, DMSO-d₆) δ 7.32 (d, J=7.8 Hz, 1H), 7.21 (dd, J=7.7, 1.7 Hz, 1H), 7.11 (d, J=1.7 Hz, 1H), 6.64 (s, 2H), 6.36 (s, 1H), 3.95-3.82 (m, 1H), 3.73 (s, 2H), 2.94-2.81 (m, 6H), 2.58 (s, 3H), 2.21 (t, J=11.5 Hz, 2H), 2.08-1.98 (m, 2H), 1.89 (d, J=12.5 Hz, 2H), 1.54 (q, J=11.5 Hz, 2H). LCMS^{AD} m/z=430 [M+H]⁺.

### Example 527-Example 682

These Examples were synthesised according to methods similar to those cited in the following table:

| | | | | LCMS |
|---|---|---|---|---|
| Example | Structure | Method Ref | ¹H NMR | m/z |
| Example 527 | 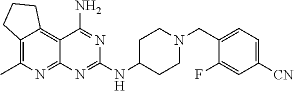 [SMILES: Cc1nc2nc(NC3CCN(Cc4ccc(C#N)cc4F)CC3)nc(N)c2c2c1CCC2] | Example 426, using Example 556 and appropriate aldehyde | ¹H NMR (400 MHz, DMSO- d₆) δ 7.82 (dd, J = 9.8, 1.6 Hz, 1H), 7.70 (dd, J = 7.9, 1.6 Hz, 1H), 7.64 (t, J = 7.5 Hz, 1H), 6.56 (s, 2H), 6.30 (s, 1H), 3.87-3.75 (m, 1H), 3.60 (s, | S: 432 [M + H]⁺ |
| | | | 2H), 3.31-3.25 (m, 2H), 2.86- | |
| | | | 2.70 (m, 4H), 2.37 (s, 3H), | |
| | | | 2.18-2.01 (m, 4H), 1.88-1.74 | |
| | | | (m, 2H), 1.51 (qd, J = 11.9 | |
| | | | 3.7 Hz, 2H). | |
| Example 528 | 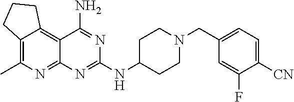 [SMILES: Cc1nc2nc(NC3CCN(Cc4ccc(C#N)c(F)c4)CC3)nc(N)c2c2c1CCC2] | Example 426, using Example 556 and appropriate aldehyde | ¹H NMR (400 MHz, DMSO- d₆) δ 7.88 (t, J = 7.4 Hz, 1H), 7.45 (d, J = 10.5 Hz, 1H), 7.38 (dd, J = 8.0, 1.3 Hz, 1H), 6.56 (s, 2H), 6.31 (s, 1H), 3.90-3.76 (m, 1H), 3.59 (s, 2H), 3.31-3.26 (m, 2H), 2.87- | S: 432 [M + H]⁺ |
| | | | 2.73 (m, 4H), 2.37 (s, 3H), | |
| | | | 2.17-2.02 (m, 4H), 1.84 (d, | |
| | | | J = 12.2 Hz, 2H), 1.62-1.46 | |
| | | | (m, 2H). | |
| Example 529 | 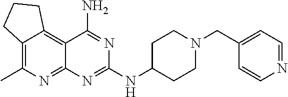 [SMILES: Cc1nc2nc(NC3CCN(Cc4ccncc4)CC3)nc(N)c2c2c1CCC2] | Example 426, using Example 556 and appropriate aldehyde | ¹H NMR (400 MHz, DMSO- d₆) δ 8.55-8.43 (m, 2H), 7.35- 7.28 (m, 2H), 6.56 (s, 2H), 6.31 (s, 1H), 3.90-3.77 (m, 1H), 3.51 (s, 2H), 3.31-3.25 (m, 2H), 2.85-2.73 (m, 4H), | W: 390 [M + H]⁺ |
| | | | 2.37 (s, 3H), 2.15-2.01 (m, | |
| | | | 4H), 1.84 (d, J = 12.0 Hz, | |
| | | | 2H), 1.53 (qd, J = 11.9, 3.8 | |
| | | | Hz, 2H). | |
| Example 530 | 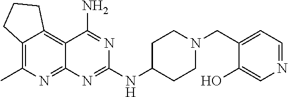 [SMILES: Cc1nc2nc(NC3CCN(Cc4ccncc4O)CC3)nc(N)c2c2c1CCC2] | Example 426, using Example 556 and appropriate aldehyde | ¹H NMR (400 MHz, DMSO- d₆) δ 8.06 (s, 1H), 7.99 (d, J = 4.8 Hz, 1H), 7.15 (d, J = 4.8 Hz, 1H), 6.58 (s, 2H), 6.37 (s, 1H), 3.96-3.82 (m, 1H), 3.67 (s, 2H), 3.30 (t, J = 7.5 Hz, | W: 406 [M + H]⁺ |
| | | | 2H), 2.95-2.84 (m, 2H), 2.81 | |
| | | | (t, J = 7.6 Hz, 2H), 2.37 (s, | |
| | | | 3H), 2.21 (t, J = 11.4 Hz, | |
| | | | 2H), 2.10 (p, J = 7.6 Hz, 2H), | |
| | | | 1.95-1.82 (m, 2H), 1.55 (qd, | |
| | | | J = 11.9, 3.7 Hz, 2H). | |
| Example 531 | 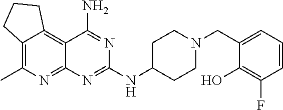 [SMILES: Cc1nc2nc(NC3CCN(Cc4cccc(F)c4O)CC3)nc(N)c2c2c1CCC2] | Example 426, using Example 556 and appropriate aldehyde | ¹H NMR (400 MHz, DMSO- d₆) δ 7.10-6.99 (m, 1H), 6.89 (d, J = 7.5 Hz, 1H), 6.79-6.69 (m, 1H), 6.58 (s, 2H), 6.39 (s, 1H), 3.97-3.86 (m, 1H), 3.76 (s, 2H), 3.30 (t, J = 7.6 Hz, 2H), 2.92 (d, J = 11.4 Hz, | W: 423 [M + H]⁺ |
| | | | 2H), 2.81 (t, J = 7.6 Hz, 2H), | |
| | | | 2.37 (s, 3H), 2.23 (t, J = 11.4 | |
| | | | Hz, 2H), 2.15-2.08 (m, 2H), | |
| | | | 1.96-1.86 (m, 2H), 1.63-1.47 | |
| | | | (m, 2H). | |
| Example 532 | 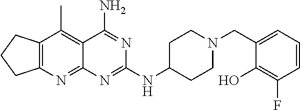 [SMILES: Cc1c2c(nc3nc(NC4CCN(Cc5cccc(F)c5O)CC4)nc(N)c13)CCC2] | Example 426, using Example 577 and appropriate aldehyde | ¹H NMR (400 MHz, DMSO- d₆) δ 7.11-6.99 (m, 1H), 6.89 (d, J = 7.6 Hz, 1H), 6.81-6.57 (m, 3H), 6.38 (s, 1H), 3.98- 3.86 (m, 1H), 3.71 (s, 2H), 3.05-2.79 (m, 6H), 2.58 (s, 3H), 2.22 (t, J = 11.3 Hz, | S: 423 [M + H]⁺ |
| | | | 2H), 2.11-1.97 (m, 2H), 1.91 | |
| | | | (d, J = 12.5 Hz, 2H), 1.65- | |
| | | | 1.44 (m, 2H). | |
| Example 533 | 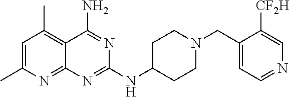 [SMILES: Cc1cc(C)c2c(N)nc(NC3CCN(Cc4ccncc4C(F)F)CC3)nc2n1] | Example 508, using appropriate starting material | ¹H NMR (400 MHz, DMSO- d₆) δ 8.75 (s, 1H), 8.69 (d, J = 5.1 Hz, 1H), 7.59-7.26 (m, 2H), 7.00-6.56 (m, 3H), 6.40 (s, 1H), 3.95-3.78 (m, 1H), 3.66 (s, 2H), 2.77 (d, J = 11.1 | AA: 414 [M + H]⁺ |
| | | | Hz, 2H), 2.64 (s, 3H), 2.38 | |
| | | | (s, 3H), 2.13 (t, J = 11.4 Hz, | |
| | | | 2H), 1.84 (d, J = 12.5 Hz, | |
| | | | 2H), 1.53 (q, J = 12.0, 11.2 | |
| | | | Hz, 2H). | |
| Example 534 | 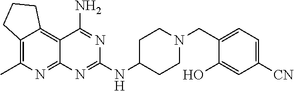 [SMILES: Cc1nc2nc(NC3CCN(Cc4ccc(C#N)cc4O)CC3)nc(N)c2c2c1CCC2] | Example 426, using Example 556 and appropriate aldehyde | ¹H NMR (400 MHz, DMSO- d₆) δ 7.32 (d, J = 7.8 Hz, 1H), 7.21 (dd, J = 7.7, 1.6 Hz, 1H), 7.11 (d, J = 1.6 Hz, 1H), 6.58 (s, 2H), 6.38 (s, 1H), 3.94- 3.84 (m, 1H), 3.74 (s, 2H), | AB: 430 [M + H]⁺ |
| | | | 3.30 (t, J = 7.6 Hz, 2H), 2.88 | |
| | | | (d, J = 11.4 Hz, 2H), 2.81 (t, | |
| | | | J = 7.6 Hz, 2H), 2.37 (s, 3H), | |
| | | | 2.22 (t, J = 11.5 Hz, 2H), 2.09 | |
| | | | (q, J = 7.6 Hz, 2H), 1.94-1.81 | |
| | | | (m, 2H), 1.61-1.47 (m, 2H). | |
| Example 535 | 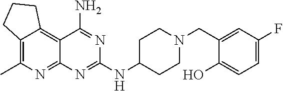 [SMILES: Cc1nc2nc(NC3CCN(Cc4cc(F)ccc4O)CC3)nc(N)c2c2c1CCC2] | Example 426, using Example 556 and appropriate aldehyde | ¹H NMR (400 MHz, DMSO- d₆) δ 7.04-6.84 (m, 2H), 6.77- 6.47 (m, 3H), 6.37 (s, 1H), 3.97-3.82 (m, 1H), 3.63 (s, 2H), 3.30 (t, J = 7.5 Hz, 2H), 2.89 (d, J = 11.4 Hz, 2H), | AV: 423 [M + H]⁺ |
| | | | 2.81 (t, J = 7.6 Hz, 2H), 2.38 | |
| | | | (s, 3H), 2.26-2.02 (m, 4H), | |
| | | | 1.89 (d, J = 12.2 Hz, 2H), | |
| | | | 1.54 (qd, J = 12.1, 3.8 Hz, | |
| | | | 2H). | |
| Example 536 | 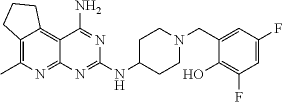 [SMILES: Cc1nc2nc(NC3CCN(Cc4cc(F)cc(F)c4O)CC3)nc(N)c2c2c1CCC2] | Example 426, using Example 556 and appropriate aldehyde | ¹H NMR (400 MHz, DMSO- d₆) δ 7.14-7.03 (m, 1H), 6.89- 6.77 (m, 1H), 6.58 (s, 2H), 6.38 (s, 1H), 3.96-3.83 (m, 1H), 3.74 (s, 2H), 3.30 (t, J = 7.6 Hz, 2H), 2.91 (d, J = 11.5 Hz, 2H), 2.81 (t, J = 7.7 Hz, | AB: 441 [M + H]⁺ |
| | | | 2H), 2.37 (s, 3H), 2.23 (t, J = | |
| | | | 11.5 Hz, 2H), 2.10 (p, J = 7.4 | |
| | | | Hz, 2H), 1.90 (d, J = 12.7 Hz, | |
| | | | 2H), 1.54 (q, J = 11.5, 11.0 | |
| | | | Hz, 2H). | |
| Example 537 | 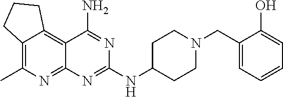 [SMILES: Cc1nc2nc(NC3CCN(Cc4ccccc4O)CC3)nc(N)c2c2c1CCC2] | Example 426, using Example 556 and appropriate aldehyde | ¹H NMR (400 MHz, DMSO- d₆) δ 10.83 (s, 1H), 7.18-7.03 (m, 2H), 6.83-6.68 (m, 2H), 6.58 (s, 2H), 6.37 (s, 1H), 3.97-3.83 (m, 1H), 3.67 (s, 2H), 3.33-3.27 (m, 2H), 2.90 (d, J = 10.8 Hz, 2H), 2.81 (t, | AB: 405 [M + H]⁺ |
| | | | J = 7.6 Hz, 2H), 2.37 (s, 3H), | |
| | | | 2.27-2.03 (m, 4H), 2.00-1.79 | |
| | | | (m, 2H), 1.64-1.45 (m, 2H). | |
| Example 538 | 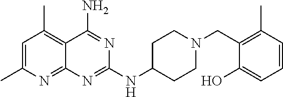 [SMILES: Cc1cc(C)c2c(N)nc(NC3CCN(Cc4c(C)cccc4O)CC3)nc2n1] | Example 426, using appropriate aldehyde | ¹H NMR (400 MHz, DMSO- d₆) δ 11.44 (s, 1H), 6.96 (t, J = 7.8 Hz, 1H), 6.89-6.61 (m, 3H), 6.59 (d, J = 7.5 Hz, 1H), 6.54 (d, J = 8.0 Hz, 1H), 6.45 (s, 1H), 3.95-3.83 (m, 1H), | S: 393 [M + H]⁺ |
| | | | 3.68 (s, 2H), 2.89 (d, J = 11.2 | |
| | | | Hz, 2H), 2.63 (s, 3H), 2.37 (s, | |
| | | | 3H), 2.30-2.13 (m, 5H), 1.95- | |
| | | | 1.82 (m, 2H), 1.60-1.44 (m, | |
| | | | 2H). | |
| Example 539 | 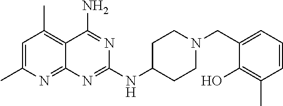 [SMILES: Cc1cc(C)c2c(N)nc(NC3CCN(Cc4cccc(C)c4O)CC3)nc2n1] | Example 426, using appropriate aldehyde | ¹H NMR (400 MHz, DMSO- d₆) δ 11.53 (s, 1H), 6.99 (d, J = 6.8 Hz, 1H), 6.86 (d, J = 6.6 Hz, 1H), 6.81-6.59 (m, 4H), 6.45 (s, 1H), 4.00-3.86 (m, 1H), 3.70 (s, 2H), 2.91 (d, J = 11.4 Hz, 2H), 2.64 (s, 3H), | S: 393 [M + H]⁺ |
| | | | 2.37 (s, 3H), 2.21 (t, J = 11.7 | |
| | | | Hz, 2H), 2.12 (s, 3H), 1.90 | |
| | | | (d, J = 12.0 Hz, 2H), 1.63- | |
| | | | 1.45 (m, 2H). | |
| Example 540 | 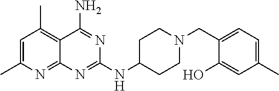 | Example 426, using appropriate aldehyde | ¹H NMR (400 MHz, DMSO- d₆) δ 10.88 (br s, 1H), 6.94 (d, J = 7.4 Hz, 1H), 6.87-6.59 (m, 3H), 6.60-6.52 (m, 2H), 6.45 (s, 1H), 3.97-3.80 (m, 1H), 3.62 (s, 2H), 2.89 (d, J = | S: 393 [M + H]⁺ |
| | | | 11.4 Hz, 2H), 2.63 (s, 3H), | |
| | | | 2.37 (s, 3H), 2.26-2.08 (m, | |
| | | | 5H), 1.88 (d, J = 12.6 Hz, | |
| | | | 2H), 1.64-1.42 (m, 2H). | |
| Example 541 | 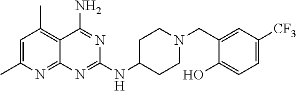 [SMILES: Cc1cc(C)c2c(N)nc(NC3CCN(Cc4cc(C(F)(F)F)ccc4O)CC3)nc2n1] | Example 426, using appropriate aldehyde | ¹H NMR (400 MHz, DMSO- d₆) δ 7.55-7.38 (m, 2H), 6.89 (d, J = 8.3 Hz, 1H), 6.82-6.56 (m, 3H), 6.45 (s, 1H), 3.95- 3.84 (m, 1H), 3.74 (s, 2H), 2.90 (d, J = 11.3 Hz, 2H), 2.63 | S: 447 [M + H]⁺ |
| | | | (s, 3H), 2.37 (s, 3H), 2.22 (t, | |
| | | | J = 11.5 Hz, 2H), 1.89 (d, J = | |
| | | | 12.2 Hz, 2H), 1.55 (q, J = | |
| | | | 11.4 Hz, 2H). | |
| Example 542 | 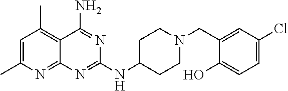 [SMILES: Cc1cc(C)c2c(N)nc(NC3CCN(Cc4cc(Cl)ccc4O)CC3)nc2n1] | Example 426, using appropriate aldehyde | ¹H NMR (400 MHz, DMSO- d₆) δ 7.23-7.06 (m, 2H), 6.93- 6.55 (m, 4H), 6.44 (s, 1H), 3.96-3.84 (m, 1H), 3.64 (s, 2H), 2.88 (d, J = 11.3 Hz, 2H), 2.63 (s, 3H), 2.37 (s, | S: 413 [M + H]⁺ |
| | | | 3H), 2.18 (t, J = 11.6 Hz, 2H), | |
| | | | 1.95-1.80 (m, 2H), 1.61-1.45 | |
| | | | (m, 2H). | |
| Example 543 | 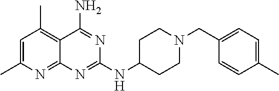 | Example 426, using appropriate aldehyde | ¹H NMR (400 MHz, DMSO- d₆) δ 7.18 (d, J = 7.8 Hz, 2H), 7.12 (d, J = 7.7 Hz, 2H), 6.90- 6.58 (m, 3H), 6.35 (s, 1H), 3.91-3.74 (m, 1H), 3.41 (s, 2H), 2.78 (d, J = 11.0 Hz, | S: 377 [M + H]⁺ |
| | | | 2H), 2.63 (s, 3H), 2.37 (s, 3H), | |
| | | | 2.28 (s, 3H), 1.99 (t, J = 11.4 | |
| | | | Hz, 2H), 1.80 (d, J = 12.3 Hz, | |
| | | | 2H), 1.58-1.39 (m, 2H). | |
| Example 544 | 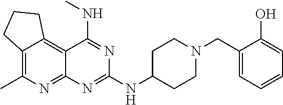 [SMILES: CNc1nc(NC2CCN(Cc3ccccc3O)CC2)nc2nc(C)c3c(c12)CCC3] | Example 426, using Example 552 and appropriate aldehyde | ¹H NMR (400 MHz, DMSO- d₆) δ 11.80 (s, 1 H), 7.14-7.04 (m, 2H), 6.78-6.68 (m, 2H), 6.61-6.41 (m, 2H), 3.97-3.80 (m, 1H), 3.66 (s, 2H), 3.32- 3.28 (m, 2H), 3.01-2.86 (m, 5H), 2.81 (t, J = 7.6 Hz, 2H), | W: 419 [M + H]⁺ |
| | | | 2.37 (s, 3H), 2.27-2.03 (m, | |
| | | | 4H), 1.97-1.82 (m, 2H), 1.54 | |
| | | | (qd, J = 12.1, 3.7 Hz, 2H). | |
| Example 545 | 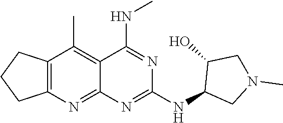 [SMILES: CNc1nc(N[C@@H]2CN(C)C[C@H]2O)nc2nc3c(c(C)c12)CCC3] | Example 232, using appropriate amine | ¹H NMR (400 MHz, DMSO- d₆) δ 6.95 (s, 1H), 6.86-6.75 (m, 1H), 6.16 (s, 1H), 4.24- 3.82 (m, 2H), 2.95 (d, J = 4.4 Hz, 3H), 2.92-2.79 (m, 5H), 2.70 (dd, J = 9.5, 6.9 Hz, 1H), 2.60 (s, 3H), 2.48-2.36 (m, 2H), 2.24-2.18 (m, 3H), 2.03 | U: 329 [M + H]⁺ |
| | | | (p, J = 7.6 Hz, 2H). | |
| Example 546 | 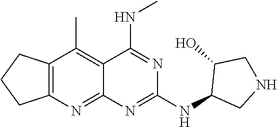 [SMILES: CNc1nc(N[C@@H]2CNC[C@H]2O)nc2nc3c(c(C)c12)CCC3] | Example 232, using appropriate amines | ¹H NMR (400 MHz, Methanol-d₄) δ 4.38-4.28 (m, 1H), 4.27-4.17 (m, 1H), 3.48 (dd, J = 12.2, 6.8 Hz, 1H), 3.29-3.25 (m, 1H), 3.13-2.88 (m, 9H), 2.66 (s, 3H), 2.15 (p, J = 7.6 Hz, 2H). | U: 315 [M + H]⁺ |
| Example 547 | 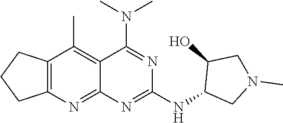 [SMILES: Cc1c2c(nc3nc(N[C@H]4CN(C)C[C@@H]4O)nc(N(C)C)c13)CCC2] | Example 232, using appropriate amines | ¹H NMR (400 MHz, Methanol-d₄) δ 4.32-4.10 (m, 2H), 3.11 (t, J = 8.7 Hz, 1H), 3.06-2.89 (m, 11H), 2.68-2.50 (m, 5H), 2.38 (s, 3H), 2.17 (p, J = 7.7 Hz, 2H). | U: 343 [M + H]⁺ |
| Example 548 | 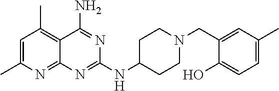 | Example 426, using appropriate aldehyde | ¹H NMR (400 MHz, DMSO- d₆) δ 10.63 (s, 1H), 7.02-6.83 (m, 2H), 6.81-6.52 (m, 4H), 6.44 (s, 1H), 4.02-3.83 (m, 1H), 3.62 (s, 2H), 2.89 (d, J = 11.3 Hz, 2H), 2.63 (s, 3H), | S: 393 [M + H]⁺ |
| | | | 2.37 (s, 3H), 2.26-2.06 (m, | |
| | | | 5H), 1.88 (d, J = 11.7 Hz, | |
| | | | 2H), 1.52 (q, J = 11.5, 10.8 | |
| | | | Hz, 2H). | |
| Example 549 | 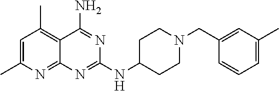 | Example 426, using appropriate aldehyde | ¹H NMR (400 MHz, DMSO- d₆) δ 7.20 (t, J = 7.5 Hz, 1H), 7.15-7.00 (m, 3H), 6.90-6.56 (m, 3H), 6.35 (s, 1H), 3.90- 3.76 (m, 1H), 3.42 (s, 2H), 2.79 (d, J = 11.0 Hz, 2H), | S: 377 [M + H]⁺ |
| | | | 2.63 (s, 3H), 2.37 (s, 3H), | |
| | | | 2.30 (s, 3H), 2.01 (t, J = 11.3 | |
| | | | Hz, 2H), 1.80 (d, J = 12.1 | |
| | | | Hz, 2H), 1.60-1.43 (m, 2H). | |
| Example 550 | 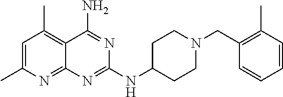 [SMILES: Cc1cc(C)c2c(N)nc(NC3CCN(Cc4ccccc4C)CC3)nc2n1] | Example 426, using appropriate aldehyde | ¹H NMR (400 MHz, DMSO- d₆) δ 7.31-7.20 (m, 1H), 7.19- 7.07 (m, 3H), 6.90-6.56 (m, 3H), 6.36 (s, 1H), 3.93-3.77 (m, 1H), 3.42 (s, 2H), 2.80 (d, J = 11.0 Hz, 2H), 2.63 (s, 3H), | S: 377 [M + H]⁺ |
| | | | 2.37 (s, 3H), 2.32 (s, 3H), | |
| | | | 2.06 (t, J = 11.3 Hz, 2H), 1.81 | |
| | | | (d, J = 12.2 Hz, 2H), 1.48 (qd, | |
| | | | J = 11.8, 3.8 Hz, 2H). | |
| Example 551 | 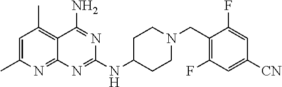 [SMILES: Cc1cc(C)c2c(N)nc(NC3CCN(Cc4c(F)cc(C#N)cc4F)CC3)nc2n1] | Example 426, using appropriate aldehyde | ¹H NMR (400 MHz, DMSO- d₆) δ 7.91-7.69 (m, 2H), 6.94- 6.51 (m, 3H), 6.34 (s, 1H), 3.84-3.69 (m, 1H), 3.62 (s, 2H), 2.79 (d, J = 10.9 Hz, 2H), 2.63 (s, 3H), 2.37 (s, | W: 424 [M + H]⁺ |
| | | | 3H), 2.12 (t, J = 11.3 Hz, | |
| | | | 2H), 1.80 (d, J = 14.6 Hz, | |
| | | | 2H), 1.53-1.40 (m, 2H). | |
| Example 552 | 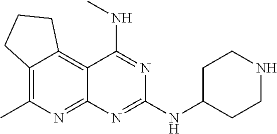 [SMILES: CNc1nc(NC2CCNCC2)nc2nc(C)c3c(c12)CCC3] | Example 230, separating other regioisomer and then Example 231, using appropriate amines | ¹H NMR (300 MHz, Methanol-d₄) δ 4.19-4.05 (m, 1H), 3.40-3.34 (m, 2H), 3.23- 3.12 (m, 2H), 3.07 (s, 3H), 2.92 (t, J = 7.6 Hz, 2H), 2.86- 2.75 (m, 2H), 2.47 (s, 3H), 2.26 (p, J = 7.6 Hz, 2H), 2.12 (d, J = 12.3 Hz, 2H), 1.63- 1.44 (m, 2H). | W: 313 [M + H]⁺ |
| Example 553 | 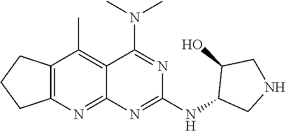 [SMILES: Cc1c2c(nc3nc(N[C@H]4CNC[C@@H]4O)nc(N(C)C)c13)CCC2] | Example 231, using appropriate amines | ¹H NMR (400 MHz, Methanol-d₄) δ 4.31-4.25 (m, 1H), 4.24-4.16 (m, 1H), 3.46 (dd, J = 12.1, 6.7 Hz, 1H), 3.24 (dd, J = 12.3, 5.3 Hz, 1H), 3.08-2.96 (m, 9H), 2.96- 2.88 (m, 3H), 2.57 (s, 3H), 2.18 (p, J = 7.5 Hz, 2H). | AK: 329 [M + H]⁺ |
| Example 554 | 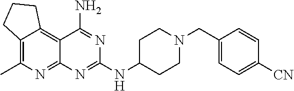 [SMILES: Cc1nc2nc(NC3CCN(Cc4ccc(C#N)cc4)CC3)nc(N)c2c2c1CCC2] | Example 426, using Example 556 and appropriate aldehyde | ¹H NMR (400 MHz, DMSO- d₆) δ 7.84-7.76 (m, 2H), 7.56- 7.48 (m, 2H), 6.64 (s, 2H), 6.44 (s, 1H), 3.95-3.78 (m, 1H), 3.57 (s, 2H), 3.30-3.20 (m, 2H), 2.91-2.71 (m, 4H), | AV: 414 [M + H]⁺ |
| | | | 2.39 (s, 3H), 2.21-2.02 (m, | |
| | | | 4H), 1.89-1.77 (m, 2H), 1.53 | |
| | | | (qd, J = 11.9, 3.7 Hz, 2H). | |
| Example 555 | 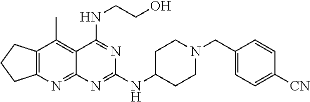 [SMILES: Cc1c2c(nc3nc(NC4CCN(Cc5ccc(C#N)cc5)CC4)nc(NCCO)c13)CCC2] | Example 230, using appropriate amines then Example 426, using appropriate aldehyde | ¹H NMR (400 MHz, DMSO- d₆) δ 7.90-7.73 (m, 2H), 7.52 (d, J = 8.1 Hz, 2H), 6.59 (s, 1H), 6.44 (s, 1H), 4.81 (t, J = 5.2 Hz, 1H), 3.87-3.70 (m, 1H), 3.67-3.60 (m, 2H), 3.58- 3.49 (m, 4H), 2.86 (q, J = 7.3 | S: 458 [M + H]⁺ |
| | | | Hz, 4H), 2.78 (d, J = 11.2 Hz, | |
| | | | 2H), 2.59 (s, 3H), 2.12-1.96 | |
| | | | (m, 4H), 1.84 (d, J = 12.3 Hz, | |
| | | | 2H), 1.59-1.46 (m, 2H). | |
| Example 556 | 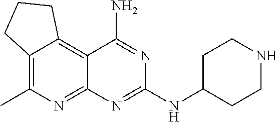 [SMILES: Cc1nc2nc(NC3CCNCC3)nc(N)c2c2c1CCC2] | Example 230, separating other regioisomer and using appropriate amine | ¹H NMR (400 MHz, DMSO- d₆) δ 6.62 (s, 2H), 6.38 (s, 1H), 4.03-3.86 (m, 1H), 3.36- 3.30 (m, 2H), 3.04 (d, J = 12.3 Hz, 2H), 2.87 (t, J = 7.6 Hz, 2H), 2.67-2.59 (m, 2H), 2.43 (s, 3H), 2.24-2.08 (m, 2H), 1.87 (d, J = 12.3 Hz, 2H), 1.42 | W: 299 [M + H]⁺ |
| | | | (qd, J = 12.0, 3.8 Hz, 2H). | |
| Example 557 | 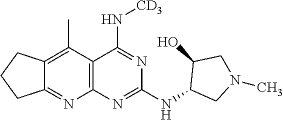 [SMILES: [2H]C([2H])([2H])Nc1nc(N[C@H]2CN(C)C[C@@H]2O)nc2nc3c(c(C)c12)CCC3] | Example 509, using appropriate amine | ¹H NMR (400 MHz, Methanol-d₄) δ 4.30-4.13 (m, 2H), 3.16-3.04 (m, 1H), 3.02- 2.88 (m, 5H), 2.63 (s, 3H), 2.61-2.51 (m, 2H), 2.37 (s, 3H), 2.13 (p, J = 7.7 Hz, 2H). | W: 332 [M + H]⁺ |
| Example 558 | 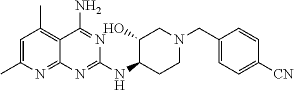 [SMILES: Cc1cc(C)c2c(N)nc(N[C@@H]3CCN(Cc4ccc(C#N)cc4)C[C@H]3O)nc2n1] | Example 426, with Example 180 and the appropriate aldehyde | ¹H NMR (400 MHz, DMSO- d₆) δ 7.81 (d, J = 8.0 Hz, 2H), 7.52 (d, J = 8.0 Hz, 2H), 6.78 (s, 2H), 6.70 (s, 1H), 6.42 (d, J = 7.0 Hz, 1H), 5.58 (s, 1H), 3.70-3.53 (m, 3H), 3.52-3.42 | S: 404 [M + H]⁺ |
| | | | (m, 1H), 2.90-2.83 (m, 1H), | |
| | | | 2.74 (d, J = 11.4 Hz, 1H), | |
| | | | 2.64 (s, 3H), 2.37 (s, 3H), | |
| | | | 2.03 (t, J = 11.7 Hz, 1H), | |
| | | | 1.96-1.82 (m, 2H), 1.49 (q, | |
| | | | J = 11.6 Hz, 1H). | |
| Example 559 | 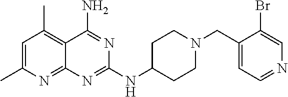 [SMILES: Cc1cc(C)c2c(N)nc(NC3CCN(Cc4ccncc4Br)CC3)nc2n1] | Example 426, with appropriate aldehyde | ¹H NMR (400 MHz, DMSO- d₆) δ 8.68 (s, 1H), 8.53 (d, J = 4.9 Hz, 1H), 7.51 (d, J = 4.9 Hz, 1H), 6.98-6.55 (m, 3H), 6.41 (s, 1H), 3.94-3.78 (m, 1H), 3.57 (s, 2H), 2.83 (d, J = | AB: 444 [M + H]⁺ |
| | | | 11.4 Hz, 2H), 2.63 (s, 3H), | |
| | | | 2.37 (s, 3H), 2.20 (t, J = 11.3 | |
| | | | Hz, 2H), 1.85 (d, J = 12.3 Hz, | |
| | | | 2H), 1.64-1.46 (m, 2H). | |
| Example 560 | 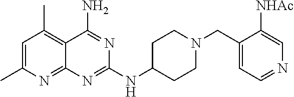 [SMILES: CC(=O)Nc1cnccc1CN1CCC(Nc2nc(N)c3c(C)cc(C)nc3n2)CC1] | Example 426, with appropriate aldehyde | ¹H NMR (400 MHz, DMSO- d₆) δ10.59 (s, 1H), 9.05 (s, 1H), 8.25 (d, J = 4.8 Hz, 1H), 7.31 (d, J = 4.9 Hz, 1H), 6.92- 6.59 (m, 3H), 6.48 (s, 1H), 3.96-3.81 (m, 1H), 3.62 (s, | S: 421 [M + H]⁺ |
| | | | 2H), 2.81 (d, J = 11.4 Hz, | |
| | | | 2H), 2.63 (s, 3H), 2.37 (s, | |
| | | | 3H), 2.19-2.06 (m, 5H), 1.88 | |
| | | | (d, J = 12.3 Hz, 2H), 1.63- | |
| | | | 1.48 (m, 2H). | |
| Example 561 | 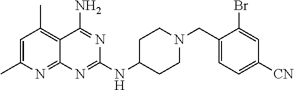 [SMILES: Cc1cc(C)c2c(N)nc(NC3CCN(Cc4ccc(C#N)cc4Br)CC3)nc2n1] | Example 426, with appropriate aldehyde | ¹H NMR (400 MHz, DMSO- d₆) δ 8.17 (d, J = 1.6 Hz, 1H), 7.88 (dd, J = 7.9, 1.7 Hz, 1H), 7.68 (d, J = 8.0 Hz, 1H), 6.68 (s, 3H), 6.40 (s, 1H), 3.96- 3.78 (m, 1H), 3.60 (s, 2H), | S: 468 [M + H]⁺ |
| | | | 2.82 (d, J = 11.4 Hz, 2H), | |
| | | | 2.63 (s, 3H), 2.37 (s, 3H), | |
| | | | 2.20 (t, J = 10.6 Hz, 2H), | |
| | | | 1.84 (d, J = 12.4 Hz, 2H), | |
| | | | 1.62-1.47 (m, 2H). | |
| Example 562 | 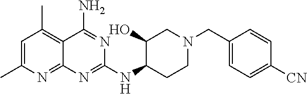 [SMILES: Cc1cc(C)c2c(N)nc(N[C@@H]3CCN(Cc4ccc(C#N)cc4)C[C@@H]3O)nc2n1] | Example 426, with Example 573 and the appropriate aldehyde | ¹H NMR (400 MHz, Methanol-d₄) δ 7.72-7.64 (m, 2H), 7.62-7.55 (m, 2H), 6.80 (s, 1H), 4.11 (d, J = 10.5 Hz, 1H), 3.93 (s, 1H), 3.62 (q, J = 13.8 Hz, 2H), 2.82 (dd, J = | Y: 404 [M + H]⁺ |
| | | | 24.9, 11.6 Hz, 2H), 2.71 (s, | |
| | | | 3H), 2.46 (s, 3H), 2.35 (d, J = | |
| | | | 11.7 Hz, 1H), 2.24 (t, J = 10.4 | |
| | | | Hz, 1H), 1.99-1.79 (m, 2H). | |
| Example 563 | 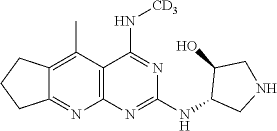 [SMILES: [2H]C([2H])([2H])Nc1nc(N[C@H]2CNC[C@@H]2O)nc2nc3c(c(C)c12)CCC3] | Example 231, using appropriate amines | ¹H NMR (400 MHz, Methanol-d₄) δ 4.29-4.17 (m, 2H), 3.43 (dd, J = 12.1, 6.8 Hz, 1H), 3.20 (dd, J = 12.3, 5.4 Hz, 1H), 3.06-2.82 (m, 6H), 2.66 (s, 3H), 2.14 (p, J = 7.7 Hz, 2H). | S: 318 [M + H]⁺ |
| Example 564 | 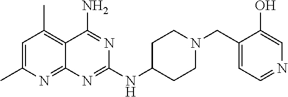 [SMILES: Cc1cc(C)c2c(N)nc(NC3CCN(Cc4ccncc4O)CC3)nc2n1] | Example 426, with appropriate aldehyde | ¹H NMR (400 MHz, Methanol-d₄) δ 8.00 (s, 1H), 7.94 (d, J = 4.9 Hz, 1H), 7.16 (d, J = 4.9 Hz, 1H), 6.79 (s, 1H), 4.13-3.98 (m, 1H), 3.81 (s, 2H), 2.97 (d, J = 11.5 Hz, | AK: 380 [M + H]⁺ |
| | | | 2H), 2.71 (d, J = 0.8 Hz, 3H), | |
| | | | 2.47 (s, 3H), 2.43-2.34 (m, | |
| | | | 2H), 2.16-2.06 (m, 2H), 1.70- | |
| | | | 1.55 (m, 2H). | |
| Example 565 | 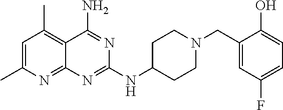 [SMILES: Cc1cc(C)c2c(N)nc(NC3CCN(Cc4cc(F)ccc4O)CC3)nc2n1] | Example 426, with appropriate aldehyde | ¹H NMR (400 MHz, Methanol-d₄) δ 6.88-6.80 (m, 2H), 6.79 (s, 1H), 6.70 (dd, J = 8.6, 4.7 Hz, 1H), 4.08-3.96 (m, 1H), 3.71 (s, 2H), 2.96 (d, J = 11.5 Hz, 2H), 2.71 (d, J = 0.8 Hz, 3H), 2.46 (s, 3H), | AK: 397 [M + H]⁺ |
| | | | 2.38-2.29 (m, 2H), 2.08 (d, | |
| | | | J = 12.8 Hz, 2H), 1.69-1.50 | |
| | | | (m, 2H). | |
| Example 566 | 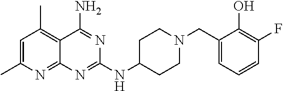 [SMILES: Cc1cc(C)c2c(N)nc(NC3CCN(Cc4cccc(F)c4O)CC3)nc2n1] | Example 426, with appropriate aldehyde | ¹H NMR (400 MHz, DMSO- d₆) δ 7.10-7.00 (m, 1H), 6.89 (d, J = 7.6 Hz, 1H), 6.82-6.57 (m, 4H), 6.46 (s, 1H), 3.98- 3.85 (m, 1H), 3.76 (s, 2 H), 2.92 (d, J = 11.4 Hz, 2H), | S: 397 [M + H]⁺ |
| | | | 2.64 (s, 3H), 2.37 (s, 3H), | |
| | | | 2.29-2.13 (m, 2H), 1.90 (d, | |
| | | | J = 12.6 Hz, 2H), 1.64-1.40 | |
| | | | (m, 2H). | |
| Example 567 | 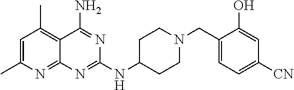 [SMILES: Cc1cc(C)c2c(N)nc(NC3CCN(Cc4ccc(C#N)cc4O)CC3)nc2n1] | Example 426, with appropriate aldehyde | ¹H NMR (400 MHz, DMSO- d₆) δ 7.32 (d, J = 7.8 Hz, 1H), 7.21 (dd, J = 7.7, 1.7 Hz, 1H), 7.11 (d, J = 1.7 Hz, 1H), 6.88-6.57 (m, 3H), 6.45 (s, 1H), 3.97-3.83 (m, 1H), 3.73 (s, 2H), 2.88 (d, J = 11.2 Hz, | S: 404 [M + H]⁺ |
| | | | 2H), 2.63 (s, 3H), 2.37 (s, | |
| | | | 3H), 2.22 (t, J = 11.5 Hz, 2H), | |
| | | | 1.89 (d, J = 12.5 Hz, 2H), | |
| | | | 1.60-1.47 (m, 2H). | |
| Example 568 | 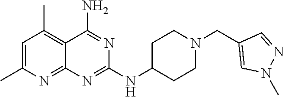 | Example 426, with appropriate aldehyde | ¹H NMR (400 MHz, DMSO- d₆) δ 7.54 (s, 1H), 7.28 (s, 1H), 6.93-6.54 (m, 3H), 6.34 (s, 1H), 3.85-3.68 (m, 4H), 3.32 (s, 2H), 2.80 (d, J = 11.1 Hz, 2H), 2.63 (s, 3H), 2.37 (s, | U: 367 [M + H]⁺ |
| | | | 3H), 2.01-1.89 (m, 2H), 1.79 | |
| | | | (d, J = 11.7 Hz, 2H), 1.47 (qd, | |
| | | | J = 12.0, 3.7 Hz, 2H). | |
| Example 569 | 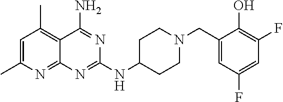 [SMILES: Cc1cc(C)c2c(N)nc(NC3CCN(Cc4cc(F)cc(F)c4O)CC3)nc2n1] | Example 426, with appropriate aldehyde | ¹H NMR (400 MHz, Methanol-d₄) δ 6.87-6.82 (m, 1H), 6.80 (s, 1H), 6.72-6.67 (m, 1H), 4.09-3.98 (m, 1H), 3.78 (s, 2H), 2.98 (d, J = 11.8 Hz, 2H), 2.71 (d, J = 0.8 Hz, 3H), 2.47 (s, 3H), 2.41-2.32 | AJ: 415 [M + H]⁺ |
| | | | (m, 2H), 2.10 (d, J = 12.9 Hz, | |
| | | | 2H), 1.71-1.54 (m, 2H). | |
| Example 570 | 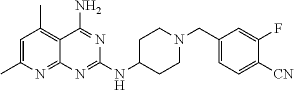 [SMILES: Cc1cc(C)c2c(N)nc(NC3CCN(Cc4ccc(C#N)c(F)c4)CC3)nc2n1] | Example 426, with appropriate aldehyde | ¹H NMR (400 MHz, DMSO- d₆) δ 7.88 (t, J = 7.4 Hz, 1H), 7.45 (d, J = 10.5 Hz, 1H), 7.38 (dd, J = 7.9, 1.3 Hz, 1H), 6.89-6.54 (m, 3H), 6.39 (s, 1H), 3.89-3.77 (m, 1H), 3.59 | S: 406 [M + H]⁺ |
| | | | (s, 2H), 2.79 (d, J = 11.5 Hz, | |
| | | | 2H), 2.63 (s, 3H), 2.37 (s, | |
| | | | 3H), 2.14-1.97 (m, 2H), 1.87- | |
| | | | 1.74 (m, 2H), 1.53 (qd, J = | |
| | | | 11.9, 3.7 Hz, 2H). | |
| Example 571 | 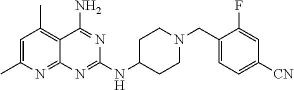 [SMILES: Cc1cc(C)c2c(N)nc(NC3CCN(Cc4ccc(C#N)cc4F)CC3)nc2n1] | Example 426, with appropriate aldehyde | ¹H NMR (400 MHz, DMSO- d₆) δ 7.82 (dd, J = 9.8, 1.5 Hz, 1H), 7.70 (dd, J = 7.9, 1.6 Hz, 1H), 7.64 (t, J = 7.5 Hz, 1H), 6.96-6.55 (m, 3H), 6.38 (s, 1H), 3.91-3.73 (m, 1H), | U: 406 [M + H]⁺ |
| | | | 3.60 (s, 2H), 2.80 (d, J = 11.2 | |
| | | | Hz, 2H), 2.63 (s, 3H), 2.37 (s, | |
| | | | 3H), 2.20-2.05 (m, 2H), 1.82 | |
| | | | (d, J = 12.3 Hz, 2H), 1.59-1.43 | |
| | | | (m, 2H). | |
| Example 572 | 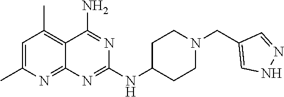 [SMILES: Cc1cc(C)c2c(N)nc(NC3CCN(Cc4cn[nH]c4)CC3)nc2n1] | Example 426, with appropriate aldehyde | ¹H NMR (400 MHz, DMSO- d₆) δ 12.55 (s, 1H), 7.48 (s, 2H), 6.89-6.55 (m, 3H), 6.33 (s, 1H), 3.85-3.71 (m, 1H), 3.39-3.35 (m, 2H), 2.80 (d, J = 11.2 Hz, 2H), 2.63 (s, 3H), | W: 353 [M + H]⁺ |
| | | | 2.37 (s, 3H), 1.94 (t, J = 11.3 | |
| | | | Hz, 2H), 1.79 (d, J = 12.1 Hz, | |
| | | | 2H), 1.56-1.40 (m, 2H). | |
| Example 573 | 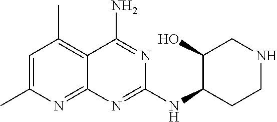 [SMILES: Cc1cc(C)c2c(N)nc(N[C@@H]3CCNC[C@@H]3O)nc2n1] | Example 225, using the appropriate amine | ¹H NMR (400 MHz, Methanol-d₄) δ 6.81 (s, 1H), 4.23-4.15 (m, 1H), 3.93 (s, 1H), 3.11-2.98 (m, 2H), 2.85 (d, J = 13.3 Hz, 1H), 2.78- 2.63 (m, 4H), 2.47 (s, 3H), 1.91-1.72 (m, 2H). | AW: 289 [M + H]⁺ |
| Example 574 | 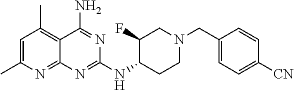 [SMILES: Cc1cc(C)c2c(N)nc(N[C@H]3CCN(Cc4ccc(C#N)cc4)C[C@@H]3F)nc2n1] | Example 426, with Example 590 and the appropriate aldehyde | ¹H NMR (400 MHz, DMSO- d₆) δ 7.85-7.76 (m, 2H), 7.56- 7.48 (m, 2H), 6.96-6.53 (m, 4H), 4.73-4.45 (m, 1H), 4.19- 4.04 (m, 1H), 3.66 (s, 2H), 3.17-3.03 (m, 1H), 2.72 (d, | S: 406 [M + H]⁺ |
| | | | J = 11.4 Hz, 1H), 2.64 (s, 3H), | |
| | | | 2.37 (s, 3H), 2.24-2.02 (m, | |
| | | | 2H), 1.95-1.83 (m, 1H), 1.60- | |
| | | | 1.41 (m, 1H). | |
| Example 575 | 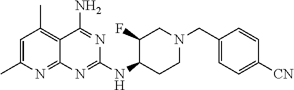 [SMILES: Cc1cc(C)c2c(N)nc(N[C@@H]3CCN(Cc4ccc(C#N)cc4)C[C@@H]3F)nc2n1] | Example 426, with Example 586 and the appropriate aldehyde | ¹H NMR (400 MHz, DMSO- d₆) δ 7.81 (d, J = 8.1 Hz, 2H), 7.53 (d, J = 8.0 Hz, 2H), 7.02- 6.75 (m, 2H), 6.72 (s, 1H), 6.26 (s, 1H), 4.83 (d, J = 49.5 Hz, 1H), 4.15-3.93 (m, 1H), | S: 406 [M + H]⁺ |
| | | | 3.70-3.56 (m, 2H), 3.08 (t, J = | |
| | | | 11.4 Hz, 1H), 2.89-2.79 (m, | |
| | | | 1H), 2.65 (s, 3H), 2.38 (s, | |
| | | | 3H), 2.34-2.11 (m, 2H), 1.99- | |
| | | | 1.78 (m, 1H), 1.70-1.60 (m, | |
| | | | 1H). | |
| Example 576 | 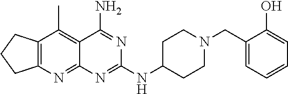 [SMILES: Cc1c2c(nc3nc(NC4CCN(Cc5ccccc5O)CC4)nc(N)c13)CCC2] | Example 426, with Example 577 and the appropriate aldehyde | ¹H NMR (400 MHz, DMSO- d₆) δ 10.74 (s, 1H), 7.15-7.02 (m, 2H), 6.86-6.51 (m, 4H), 6.36 (s, 1H), 3.94-3.79 (m, 1H), 3.66 (s, 2H), 2.99-2.77 (m, 6H), 2.57 (s, 3H), 2.17 | AA: 405 [M + H]⁺ |
| | | | (td, J = 11.9, 2.5 Hz, 2H), | |
| | | | 2.02 (p, J = 7.6 Hz, 2H), 1.95- | |
| | | | 1.81 (m, 2H), 1.53 (qd, J = | |
| | | | 11.9, 3.7 Hz, 2H). | |
| Example 577 | 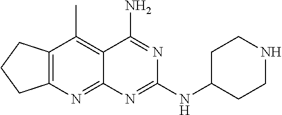 [SMILES: Cc1c2c(nc3nc(NC4CCNCC4)nc(N)c13)CCC2] | Example 230, with the appropriate amine | ¹H NMR (400 MHz, Methanol-d₄) δ 4.15-4.04 (m, 1H), 3.24 (dt, J = 13.4, 4.1 Hz, 2H), 3.06-2.87 (m, 6H), 2.66 (s, 3H), 2.19-2.08 (m, 4H), 1.67-1.52 (m, 2H). | W: 299 [M + H]⁺ |
| Example 578 | 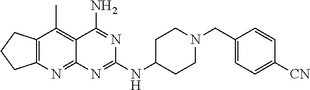 [SMILES: Cc1c2c(nc3nc(NC4CCN(Cc5ccc(C#N)cc5)CC4)nc(N)c13)CCC2] | Example 426, with Example 577 and the appropriate aldehyde | ¹H NMR (400 MHz, DMSO- d₆) δ 7.83-7.74 (m, 2H), 7.55- 7.46 (m, 2H), 6.62 (s, 2H), 6.29 (s, 1H), 3.87-3.74 (m, 1H), 3.56 (s, 2H), 2.85 (q, J = 8.1 Hz, 4H), 2.81-2.73 (m, | AB: 414 [M + H]⁺ |
| | | | 2H), 2.57 (s, 3H), 2.15-1.95 | |
| | | | (m, 4H), 1.88-1.75 (m, 2H), | |
| | | | 1.51 (qd, J = 11.9, 3.7 Hz, | |
| | | | 2H). | |
| Example 579 | 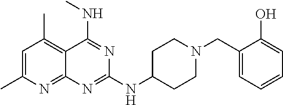 [SMILES: CNc1nc(NC2CCN(Cc3ccccc3O)CC2)nc2nc(C)cc(C)c12] | Example 426, with Example 588 and the appropriate aldehyde | ¹H NMR (400 MHz, DMSO- d₆) δ 10.94 (br, 1H), 7.15- 7.04 (m, 2H), 6.82-6.52 (m, 5H), 3.97-3.81 (m, 1H), 3.66 (s, 2H), 2.99-2.85 (m, 5H), 2.64 (s, 3H), 2.37 (s, 3H), 2.18 (t, J = 11.5 Hz, 2H), | S: 393 [M + H]⁺ |
| | | | 1.97-1.83 (m, 2H), 1.61-1.45 | |
| | | | (m, 2H). | |
| Example 580 | 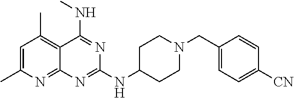 [SMILES: CNc1nc(NC2CCN(Cc3ccc(C#N)cc3)CC2)nc2nc(C)cc(C)c12] | Example 426, with Example 588 and the appropriate aldehyde | ¹H NMR (400 MHz, DMSO- d₆) δ 7.79 (d, J = 7.9 Hz, 2H), 7.52 (d, J = 8.0 Hz, 2H), 6.86-6.63 (m, 2H), 6.56 (s, 1H), 3.90-3.77 (m, 1H), 3.56 (s, 2H), 2.93 (d, J = 4.3 Hz, | S: 402 [M + H]⁺ |
| | | | 3H), 2.79 (d, J = 11.3 Hz, 2H), | |
| | | | 2.64 (s, 3H), 2.36 (s, 3H), | |
| | | | 2.08 (t, J = 11.3 Hz, 2H), 1.84 | |
| | | | (d, J = 11.1 Hz, 2H), 1.58- | |
| | | | 1.42 (m, 2H). | |
| Example 581 | 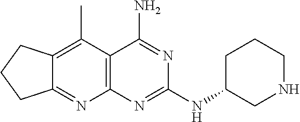 [SMILES: Cc1c2c(nc3nc(N[C@@H]4CCCNC4)nc(N)c13)CCC2] | Example 230, with the appropriate amine | ¹H NMR (400 MHz, DMSO- d₆) δ 6.67 (s, 2H), 6.30 (s, 1H), 3.99-3.86 (m, 1H), 3.13- 3.04 (m, 1H), 2.93-2.79 (m, 5H), 2.58 (s, 3H), 2.56-2.51 (m, 1H), 2.49-2.42 (m, 1H), 2.02 (p, J = 7.7 Hz, 2H), 1.89- 1.80 (m, 1H), 1.73-1.63 (m, | W: 299 [M + H]⁺ |
| | | | 1H), 1.52-1.38 (m, 2H). | |
| Example 582 | 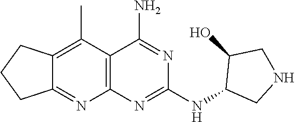 [SMILES: Cc1c2c(nc3nc(N[C@H]4CNC[C@@H]4O)nc(N)c13)CCC2] | Example 230, with the appropriate amine | ¹H NMR (400 MHz, Methanol-d₄) δ 4.34-4.26 (m, 1H), 4.22-4.12 (m, 1H), 3.49 (dd, J = 12.2, 6.9 Hz, 1H), 3.29-3.24 (m, 1H), 3.09-2.88 (m, 6H), 2.66 (s, 3H), 2.15 (p, J = 7.7 Hz, 2H). | AJ: 301 [M + H]⁺ |
| Example 583 | 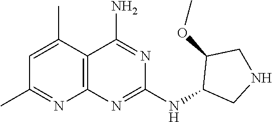 [SMILES: CO[C@H]1CNC[C@@H]1Nc1nc(N)c2c(C)cc(C)nc2n1] | Example 227, with the appropriate amine | ¹H NMR (400 MHz, Methanol-d₄) δ 6.83 (s, 1H), 4.50-4.43 (m, 1H), 3.97-3.89 (m, 1H), 3.44 (s, 3H), 3.42- 3.36 (m, 1H), 3.29-3.24 (m, 1H), 3.00 (d, J = 12.4 Hz, 2H), 2.73 (s, 3H), 2.49 (s, 3H). | AD: 289 [M + H]⁺ |
| Example 584 | 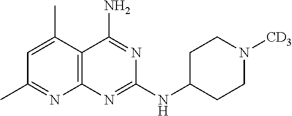 [SMILES: [2H]C([2H])([2H])N1CCC(Nc2nc(N)c3c(C)cc(C)nc3n2)CC1] | Example 512, with Example 279 | ¹H NMR (400 MHz, DMSO- d₆) δ 6.94-6.57 (m, 3H), 6.36 (s, 1H), 3.84-3.69 (m, 1H), 2.82-2.68 (m, 2H), 2.63 (s, 3H), 2.37 (s, 3H), 1.95 (td, J = 11.7, 2.4 Hz, 2H), 1.86-1.75 (m, 2H), 1.50 (qd, J = 11.8, 3.8 Hz, 2H). | S: 290 [M + H]⁺ |
| Example 585 | 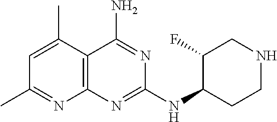 [SMILES: Cc1cc(C)c2c(N)nc(N[C@@H]3CCNC[C@H]3F)nc2n1] | Example 227, using appropriate amines | ¹H NMR (400 MHz, DMSO- d₆) δ 7.01-6.51 (m, 4H), 4.54- 4.29 (m, 1H), 4.20-4.05 (m, 1H), 3.24-3.16 (m, 1H), 2.84 (d, J = 12.5 Hz, 1H), 2.64 (s, 3H), 2.48-2.41 (m, 2H), 2.37 (s, 3H), 1.92-1.80 (m, 1H), 1.37 (qd, J = 11.8, 4.1 Hz, | AV: 291 [M + H]⁺ |
| | | | 1H). | |
| Example 586 | 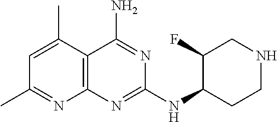 [SMILES: Cc1cc(C)c2c(N)nc(N[C@@H]3CCNC[C@@H]3F)nc2n1] | Example 227, using appropriate amines | ¹H NMR (400 MHz, DMSO- d₆) δ 6.77 (s, 2H), 6.72 (s, 1H), 6.21 (s, 1H), 4.71 (d, J = 50.5 Hz, 1H), 4.18-4.00 (m, 1H), 3.19-3.08 (m, 1H), 2.96 (d, J = 13.0 Hz, 1H), 2.85- 2.66 (m, 1H), 2.65 (s, 3H), 2.60-2.52 (m, 1H), 2.38(s, 3 | AV: 291 [M + H]⁺ |
| | | | H), 1.74-1.62 (m, 1H), 1.61- | |
| | | | 1.51 (m, 1H). | |
| Example 587 | 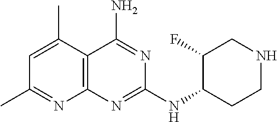 [SMILES: Cc1cc(C)c2c(N)nc(N[C@H]3CCNC[C@H]3F)nc2n1] | Example 227, using appropriate amines | ¹H NMR (400 MHz, DMSO- d₆) δ 6.77 (s, 2H), 6.72 (s, 1H), 6.22 (s, 1H), 4.71 (d, J = 50.6 Hz, 1H), 4.20-3.97 (m, 1H), 3.13 (t, J = 12.3 Hz, 1H), 2.96 (d, J = 13.0 Hz, 1H), 2.83-2.67 (m, 1H), 2.65 (s, 3H), 2.60-2.53 (m, 1H), 2.38 | AK: 291 [M + H]⁺ |
| | | | (s, 3H), 1.68 (qd, J = 12.2, 4.1 | |
| | | | Hz, 1H), 1.61-1.53 (m, 1H). | |
| Example 588 | 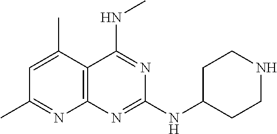 [SMILES: CNc1nc(NC2CCNCC2)nc2nc(C)cc(C)c12] | Example 227, using appropriate amine | ¹H NMR (400 MHz, DMSO- d₆) δ 6.83-6.61 (m, 2H), 6.53 (s, 1H), 3.95-3.77 (m, 1H), 3.02-2.87 (m, 5H), 2.64 (s, 3H), 2.57-2.51 (m, 2H), 2.36 (s, 3H), 1.89-1.70 (m, 2H), 1.34 (qd, J = 12.0, 4.0 Hz, 2H). | W: 287 [M + H]⁺ |
| Example 589 | 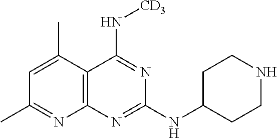 [SMILES: [2H]C([2H])([2H])Nc1nc(NC2CCNCC2)nc2nc(C)cc(C)c12] | Example 227, using appropriate amines | ¹H NMR (400 MHz, DMSO- d₆) δ 6.84-6.60 (m, 2H), 6.52 (s, 1H), 3.87 (s, 1H), 2.95 (d, J = 12.1 Hz, 2H), 2.64 (s, 3H), 2.55 (s, 2H), 2.36 (s, 3H), 1.79 (t, J = 12.1 Hz, 2H), 1.35 (qd, J = 11.8, 4.0 Hz, 2H). | W: 290 [M + H]⁺ |
| Example 590 | 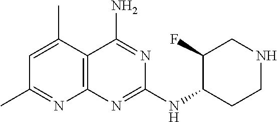 [SMILES: Cc1cc(C)c2c(N)nc(N[C@H]3CCNC[C@@H]3F)nc2n1] | Example 227, using appropriate amines | ¹H NMR (400 MHz, Methanol-d₄) δ 6.79 (s, 1H), 4.62-4.39 (m, 1H), 4.36-4.25 (m, 1H), 3.29-3.21 (m, 1H), 3.02-2.92 (m, 1H), 2.78-2.63 (m, 5H), 2.47 (s, 3H), 2.23- 2.09 (m, 1H), 1.60-1.46 (m, 1H). | W: 291 [M + H]⁺ |
| Example 591 | 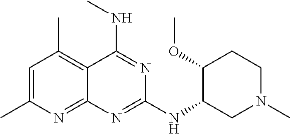 | Example 228, using appropriate amine | ¹H NMR (400 MHz, DMSO- d₆) δ 6.96-6.73 (m, 1H), 6.71 (s, 1H), 5.89-5.47 (m, 1H), 4.28 (s, 1H), 3.50-3.41 (m, 1H), 3.29 (s, 3H), 2.94 (d, J = 4.3 Hz, 3H), 2.65 (s, 3H), 2.38 (s, 3H), 2.48-2.42 (m, 1H), 2.33-2.20 (m, 3H), 2.16 (s, 3H), 1.99-1.84 (m, 1H), | W: 331 [M + H]⁺ |
| | | | 1.65-1.53 (m, 1H). | |
| Example 592 | 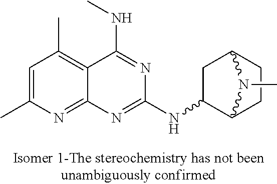 | Example 228, using Example 518 | ¹H NMR (400 MHz, Methanol-d₄) δ 6.75 (s, 1H), 4.54-4.33 (m, 1H), 3.92-3.58 (m, 1H), 3.25 (t, J = 4.7 Hz, 1H), 3.07 (s, 3H), 2.70 (d, J = 0.9 Hz, 3H), 2.45 (s, 3H), 2.42-2.19 (m, 4H), 1.98-1.82 (m, 2H), 1.78-1.61 (m, 1H), 1.61-1.48 (m, 1H), 1.24-1.09 (m, 1H). | AK: 313 [M + H]⁺ |
| Example 593 | 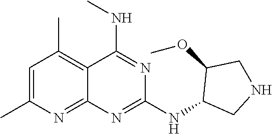 [SMILES: CNc1nc(N[C@H]2CNC[C@@H]2OC)nc2nc(C)cc(C)c12] | Example 256, using appropriate amine | ¹H NMR (400 MHz, Methanol-d₄) δ 6.77 (s, 1H), 5.74 (s, 1H), 4.53-4.44 (m, 1H), 3.89-3.82 (m, 1H), 3.49- 3.36 (m, 4H), 3.17 (dd, J = 12.6, 5.2 Hz, 1H), 2.95 (dd, J = 12.5, 2.6 Hz, 1H), 2.89 (s, 3H), 2.86-2.81 (m, 1H), 2.77 (s, 3H), 2.48 (s, 3H). | W: 302 [M + H]⁺ |
| Example 594 | 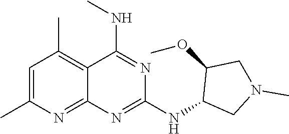 | Example 256, using appropriate amine | ¹H NMR (400 MHz, DMSO- d₆) δ 6.86 (d, J = 7.8 Hz, 1H), 6.65 (s, 1H), 5.85 (q, J = 4.2 Hz, 1H), 5.70 (s, 1H), 4.40 (s, 1H), 3.78-3.69 (m, 1H), 3.28 (s, 3H), 2.90-2.78 (m, 2H), 2.75 (d, J = 4.7 Hz, 3H), 2.70 (s, 3H), 2.42-2.36 (m, 4H), 2.28 (dd, J = 9.3, 4.9 Hz, 1H), 2.23 (s, 3H). | W: 316 [M + H]⁺ |
| Example 595 | 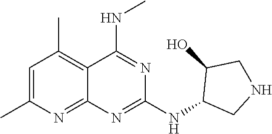 [SMILES: CNc1nc(N[C@H]2CNC[C@@H]2O)nc2nc(C)cc(C)c12] | Example 256, using appropriate amine, deprotect as per Example 140 | ¹H NMR (400 MHz, Methanol-d₄) δ 6.80 (s, 1H), 5.74 (s, 1H), 4.32-4.20 (m, 2H), 3.52 (dd, J = 12.2, 7.0 Hz, 1H), 3.22 (dd, J = 12.2, 5.2 Hz, 1H), 3.02-2.92 (m, 2H), 2.88 (s, 3H), 2.78 (s, 3H), 2.50 (s, 3H). | W: 288 [M + H]⁺ |
| Example 596 | 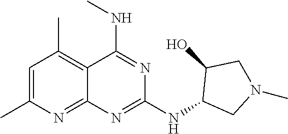 | Example 256, using appropriate amine | ¹H NMR (400 MHz, Methanol-d₄) δ 6.77 (s, 1H), 5.76 (s, 1H), 4.27-4.18 (m, 1H), 4.17-4.08 (m, 1H), 3.09 (dd, J = 10.0, 7.3 Hz, 1H), 2.93 (dd, J = 10.2, 6.4 Hz, 1H), 2.87 (s, 3H), 2.77 (s, 3H), 2.59-2.53 (m, 2H), 2.48 (s, 3H), 2.38 (s, 3H). | W: 302 [M + H]⁺ |
| Example 597 | 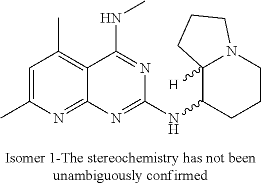 [SMILES: CNc1nc(NC2CCCN3CCCC23)nc2nc(C)cc(C)c12] | Example 259 & Example 260, using approrpate amines Purified by Chiral-HPLCB RT = 11.0 min | ¹H NMR (400 MHz, DMSO- d₆) δ 6.94-6.45 (m, 3H), 3.93- 3.75 (m, 1H), 3.12-2.86 (m, 5H), 2.64 (s, 3H), 2.36 (s, 3H), 2.07 (q, J = 8.7 Hz, 1H), 1.98-1.75 (m, 4H), 1.70-1.40 (m, 5H), 1.21-1.11 (m, 1H). | AT: 327 [M + H]⁺ |
| Example 598 | 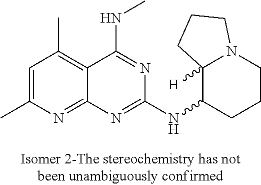 [SMILES: CNc1nc(NC2CCCN3CCCC23)nc2nc(C)cc(C)c12] | Example 259 & Example 260, using approrpate amines Purified by Chiral-HPLCB RT = 9.8 min | ¹H NMR (400 MHz, DMSO- d₆) δ 6.87-6.48 (m, 3H), 3.91- 3.80 (m, 1H), 3.03-2.89 (m, 5H), 2.64 (s, 3H), 2.36 (s, 3H), 2.06 (q, J = 8.7 Hz, 1H), 1.96-1.74 (m, 4H), 1.69-1.39 (m, 5H), 1.22-1.10 (m, 1H). | AT: 327 [M + H]⁺ |
| Example 599 | 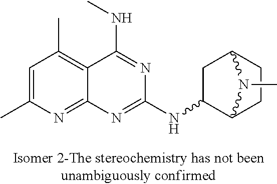 | Example 228, using Example 517 | ¹H NMR (400 MHz, Methanol-d₄) δ 6.77 (s, 1H), 4.44 (s, 1H), 3.93-3.52 (m, 1H), 3.26 (t, J = 4.7 Hz, 1H), 3.07 (s, 3H), 2.70 (s, 3H), 2.46 (s, 3H), 2.42-2.26 (m, 4H), 1.99-1.85 (m, 2H), 1.78- 1.63 (m, 1H), 1.60-1.47 (m, 1H), 1.26-1.11 (m, 1H). | AA: 313 [M + H]⁺ |
| Example 600 | 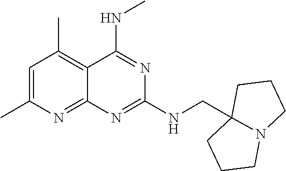 [SMILES: CNc1nc(NCC23CCCN2CCC3)nc2nc(C)cc(C)c12] | Example 227, using appropriate amine | ¹H NMR (400 MHz, DMSO- d₆) δ 6.96-6.57 (m, 2H), 6.46- 5.79 (m, 1H), 3.27 (d, J = 6.2 Hz, 2H), 3.01-2.87 (m, 5H), 2.64 (s, 3H), 2.58-2.51 (m, 2H), 2.36 (s, 3H), 1.93-1.80 (m, 2H), 1.79-1.63 (m, 4H), 1.52-1.36 (m, 2H). | AA: 327 [M + H]⁺ |
| Example 601 | 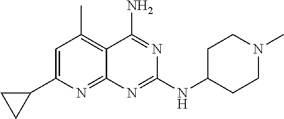 [SMILES: Cc1cc(C2CC2)nc2nc(NC3CCN(C)CC3)nc(N)c12] | Example 521, using appropriate reagent | ¹H NMR (400 MHz, DMSO- d₆) δ 6.71 (s, 3H), 6.32 (s, 1H), 3.87-3.71 (m, 1H), 2.74 (d, J = 11.6 Hz, 2H), 2.63 (s, 3H), 2.17 (s, 3H), 2.06-1.93 (m, 3H), 1.86-1.71 (m, 2H), 1.50 (q, J = 11.0 Hz, 2H), 1.03-0.89 (m, 4H). | W: 313 [M + H]⁺ |
| Example 602 | 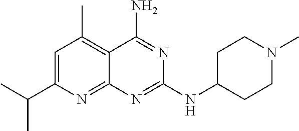 [SMILES: Cc1cc(C(C)C)nc2nc(NC3CCN(C)CC3)nc(N)c12] | Example 521, using appropriate reagent | ¹H NMR (400 MHz, Methanol-d₄) δ 8.44 (s, 2H), 7.08 (s, 1H), 4.30-4.15 (m, 1H), 3.44 (d, J = 12.4 Hz, 2H), 3.13-2.97 (m, 3H), 2.81 (s, 3H), 2.80 (s, 3H), 2.25 (d, J = 13.6 Hz, 2H), 1.96-1.81 (m, 2H), 1.32 (d, J = 6.8 Hz, 6H). Di-formate salt | AB: 315 [M + H]⁺ |
| Example 603 | 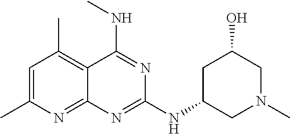 | Example 228, using appropriate amine | ¹H NMR (400 MHz, DMSO- d₆) δ 6.92-6.66 (m, 2H), 6.60 (d, J = 8.4 Hz, 1H), 4.78 (s, 1H), 4.11-3.95 (m, 1H), 3.65- 3.51 (m, 1H), 2.93 (d, J = 4.3 Hz, 3H), 2.89-2.72 (m, 2H), 2.64 (s, 3H), 2.37 (s, 3H), 2.18 (s, 3H), 2.09-1.94 (m, 1H), 1.72-1.57 (m, 2H), 1.21 | AS: 317 [M + H]⁺ |
| | | | (q, J = 11.3 Hz, 1H). | |
| Example 604 | 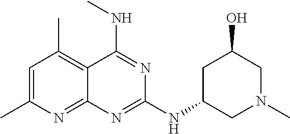 | Example 228, using appropriate amine | ¹H NMR (400 MHz, Methanol-d₄) δ 6.76 (s, 1H), 4.58-4.42 (m, 1H), 4.06-3.91 (m, 1H), 3.07 (s, 3H), 2.82- 2.65 (m, 4H), 2.64-2.41 (m, 5H), 2.37-2.10 (m, 4H), 2.07- 1.82 (m, 1H), 1.78-1.61 (m, 1H). | AK: 317 [M + H]⁺ |
| Example 605 | 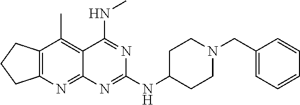 [SMILES: CNc1nc(NC2CCN(Cc3ccccc3)CC2)nc2nc3c(c(C)c12)CCC3] | Example 426, using Example 655 and appropriate aldehyde | ¹H NMR (400 MHz, DMSO- d₆) δ 7.36-7.21 (m, 5H), 6.70 (s, 1H), 6.44 (s, 1H), 3.90- 3.71 (m, 1H), 3.46 (s, 2H), 2.93 (d, J = 4.4 Hz, 3H), 2.90-2.75 (m, 6H), 2.57 (s, 3H), 2.10-1.94 (m, 4H), 1.84 | AF: 403 [M + H]⁺ |
| | | | (d, J = 12.4 Hz, 2H), 1.50 (qd, | |
| | | | J = 12.0, 3.7 Hz, 2H). | |
| Example 606 | 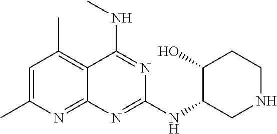 [SMILES: CNc1nc(N[C@H]2CNCC[C@H]2O)nc2nc(C)cc(C)c12] | Example 227, using appropriate amine | ¹H NMR (300 MHz, Methanol-d₄) δ 6.82 (s, 1H), 4.32-4.19 (m, 1H), 4.18-4.05 (m, 1H), 3.17-2.98 (m, 6H), 2.90-2.79 (m, 1H), 2.73 (s, 3H), 2.49 (s, 3H), 1.87 (q, J = 5.2 Hz, 2H). | AK: 303 [M + H]⁺ |
| Example 607 | 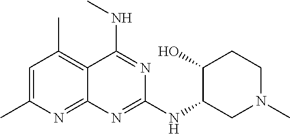 | Example 228, using appropriate amine | ¹H NMR (300 MHz, Methanol-d₄) δ 6.79 (s, 1H), 4.43-4.30 (m, 1H), 4.06-3.97 (m, 1H), 3.10 (s, 3H), 2.86- 2.66 (m, 4H), 2.62-2.37 (m, 6H), 2.32 (s, 3H), 1.96-1.80 (m, 2H). | AK: 317 [M + H]⁺ |
| Example 608 | 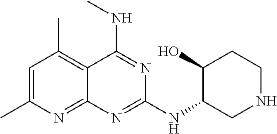 [SMILES: CNc1nc(N[C@H]2CNCC[C@@H]2O)nc2nc(C)cc(C)c12] | Example 227, using appropriate amine | ¹H NMR (400 MHz, DMSO- d₆) δ 6.80 (s, 1H), 6.70 (s, 1H), 6.41 (d, J = 7.2 Hz, 1H), 4.82 (s, 1H), 3.74-3.61 (m, 1H), 3.51-3.43 (m, 1H), 3.22- 3.17 (m, 1H), 2.95 (d, J = 4.3 Hz, 3H), 2.93-2.86 (m, 1H), 2.65 (s, 3H), 2.48-2.41 (m, 1H), 2.37 (s, 3H), 2.33-2.25 | AC: 303 [M + H]⁺ |
| | | | (m, 1H), 1.88-1.78 (m, 1H), | |
| | | | 1.44-1.29 (m, 1H). | |
| Example 609 | 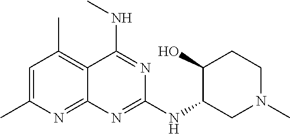 | Example 228, using appropriate amine | ¹H NMR (400 MHz, DMSO- d₆) δ 6.75 (s, 1H), 6.69 (s, 1H), 6.29 (d, J = 7.5 Hz, 1H), 5.01 (s, 1H), 3.90-3.76 (m, 1H), 3.49-3.37 (m, 1H), 3.02- 2.86 (m, 4H), 2.71-2.59 (m, 4H), 2.37 (s, 3H), 2.16 (s, 3H), 1.96 (t, J = 10.9 Hz, 1H), 1.87-1.76 (m, 2H), | W: 317 [M + H]⁺ |
| | | | 1.59-1.41 (m, 1H). | |
| Example 610 | 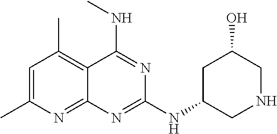 [SMILES: CNc1nc(N[C@H]2CNC[C@@H](O)C2)nc2nc(C)cc(C)c12] | Example 227, using appropriate amine | ¹H NMR (400 MHz, Methanol-d₄) δ 6.76 (s, 1H), 4.21-4.07 (m, 1H), 3.84-3.65 (m, 1H), 3.18 (dd, J = 12.3, 4.1 Hz, 1H), 3.12-2.99 (m, 4H), 2.70 (d, J = 0.8 Hz, 3H), 2.54-2.39 (m, 5H), 2.34-2.26 (m, 1H), 1.56-1.42 (m, 1H). | AQ: 303 [M + H]⁺ |
| Example 611 | 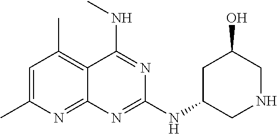 [SMILES: CNc1nc(N[C@H]2CNC[C@H](O)C2)nc2nc(C)cc(C)c12] | Example 227, using appropriate amine | ¹H NMR (400 MHz, Methanol-d₄) δ 6.77 (s, 1H), 4.48-4.35 (m, 1H), 4.02-3.92 (m, 1H), 3.19 (dd, J = 12.6, 4.0 Hz, 1H), 3.06 (s, 3H), 2.88-2.74 (m, 2H), 2.70 (s, 3H), 2.61 (dd, J = 12.7, 8.6 Hz, 1H), 2.46 (s, 3H), 2.09- 2.00 (m, 1H), 1.90-1.80 (m, | AK: 303 [M + H]⁺ |
| | | | 1H) | |
| Example 612 | 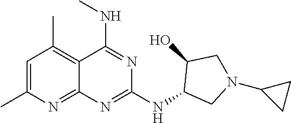 [SMILES: CNc1nc(N[C@H]2CN(C3CC3)C[C@@H]2O)nc2nc(C)cc(C)c12] | Example 519, using Example 325 | ¹H NMR (400 MHz, Methanol-d₄) δ 6.92 (s, 1H), 4.42-3.99 (m, 2H), 3.29-3.22 (m, 2H), 3.09 (s, 3H), 2.93- 2.81 (m, 1H), 2.76-2.66 (m, 4H), 2.50 (s, 3H), 1.92-1.84 (m, 1H), 0.74-0.36 (m, 4H). | AN: 329 [M + H]⁺ |
| Example 613 | 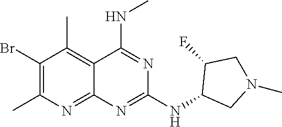 | Example 228, using appropriate amine, then Example 130, using NBS | ¹H NMR (400 MHz, DMSO- d₆) δ 7.38-7.01 (m, 1H), 6.99-6.77 (m, 1H), 5.17 (ddt, J = 55.7, 4.9, 2.5 Hz, 1H), 4.63-4.47 (m, 1H), 3.27 (dd, J = 12.1, 4.6 Hz, 1H), 3.19 (dd, J = 12.0, 4.7 Hz, 1H), 3.03-2.91 (m, 4H), 2.78 (s, | AI: 385 [M + H]⁺ |
| | | | 3H), 2.74-2.66 (m, 1H), 2.60 | |
| | | | (s, 3H), 2.38 (s, 3H). | |
| Example 614 | 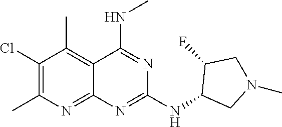 | Example 228, using appropriate amine, then Example 130 | ¹H NMR (400 MHz, DMSO- d₆) δ 7.25-6.99 (m, 1H), 6.89-6.69 (m, 1H), 5.14 (ddt, J = 55.9, 5.1, 2.5 Hz, 1H), 4.58-4.41 (m, 1H), 3.23-3.08 (m, 1H), 2.96 (d, J = 4.3 Hz, 3H), 2.88 (t, J = 8.2 Hz, 1H), 2.74 (s, 3H), 2.67-2.52 (m, | AN: 339 [M + H]⁺ |
| | | | 5H), 2.30 (s, 3H). | |
| Example 615 | 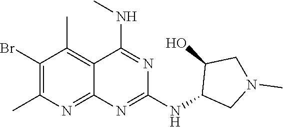 | Example 228, using appropriate amine, then Example 130, using NBS | ¹H NMR (400 MHz, DMSO- d₆) δ 7.19 (s, 1H), 7.08 (s, 1H), 5.77 (s, 1H), 4.22-3.89 (m, 2H), 2.95 (d, J = 4.4 Hz, 3H), 2.90-2.82 (m, 1H), 2.77 (s, 3H), 2.74-2.68 (m, 1H), 2.59 (s, 3H), 2.47-2.34 (m, 2H), 2.21 (s, 3H). | AR: 383 [M + H]⁺ |
| Example 616 | 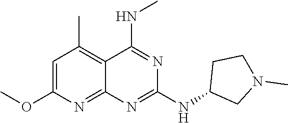 | Example 524, using sodium methoxide | ¹H NMR (400 MHz, DMSO- d₆) δ 7.03-6.47 (m, 2H), 6.27 (s, 1H), 4.54-4.38 (m, 1H), 3.84 (s, 3H), 2.93 (d, J = 4.3 Hz, 3H), 2.87-2.77 (m, 1H), 2.69-2.55 (m, 4H), 2.49-2.44 (m, 1H), 2.38 (s, 1H), 2.28 (s, 3H), 2.22-2.07 (m, 1H), 1.79 | W: 303 [M + H]⁺ |
| | | | 1.62 (m, 1H). | |
| Example 617 | 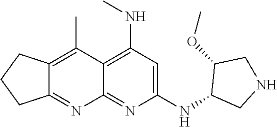 [SMILES: CNc1cc(N[C@H]2CNC[C@H]2OC)nc2nc3c(c(C)c12)CCC3] | Example 425, using appropriate amine, deprotect as per Example 140 | ¹H NMR (400 MHz, Methanol-d₄) δ 5.83 (d, J = 4.6 Hz, 1H), 4.63 (q, J = 7.6 Hz, 1H), 3.98 (td, J = 4.8, 2.5 Hz, 1H), 3.44-3.34 (m, 4H), 3.16 (dd, J = 10.2, 3.5 Hz, 2H), 3.05-2.92 (m, 4H), 2.90 (s, 3H), 2.82 (dd, J = 11.1, 8.7 Hz, 1H), 2.72 (s, 3H), 2.14 (p, J = 7.7 Hz, 2H). | AA: 328 [M + H]⁺ |
| Example 618 | 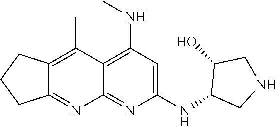 [SMILES: CNc1cc(N[C@H]2CNC[C@H]2O)nc2nc3c(c(C)c12)CCC3] | Example 425, using appropriate amine, deprotect as per Example 140 | ¹H NMR (400 MHz, Methanol-d₄) δ 5.81 (s, 1H), 4.52-4.45 (m, 1H), 4.40-4.34 (m, 1H), 3.39-3.32 (m, 1H), 3.25 (dd, J = 12.3, 4.9 Hz, 1H), 3.04-2.93 (m, 5H), 2.91- 2.84 (m, 4H), 2.71 (s, 3H), 2.18-2.07 (m, 2H). | AK: 314 [M + H]⁺ |
| Example 619 | 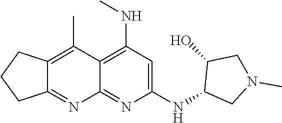 [SMILES: CNc1cc(N[C@H]2CN(C)C[C@H]2O)nc2nc3c(c(C)c12)CCC3] | Example 425, using appropriate amine | ¹H NMR (400 MHz, DMSO- d₆) δ 6.17 (d, J = 7.4 Hz, 1H), 5.88-5.77 (m, 2H), 5.07 (s, 1H), 4.39 (p, J = 7.2 Hz, 1H), 4.26-4.16 (m, 1H), 3.05-2.98 (m, 1H), 2.97-2.92 (m, 1H), 2.91-2.81 (m, 4H), 2.75 (d, J = 4.6 Hz, 3H), 2.63 (s, 3H), | AK: 328 [M + H]⁺ |
| | | | 2.32-2.22 (m, 5H), 2.02 (p, J = | |
| | | | 7.6 Hz, 2H). | |
| Example 620 | 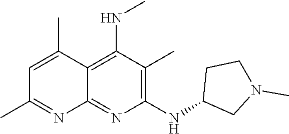 | Example 522, using appropriate amine | ¹H NMR (400 MHz, DMSO- d₆) δ 8.32 (s, 2H), 6.82 (s, 1H), 6.78-6.68 (m, 1H), 4.79- 4.65 (m, 1H), 3.40-3.23 (m, 2H), 3.20-3.07 (m, 1H), 3.00- 2.88 (m, 1H), 2.77 (s, 3H), 2.71 (s, 3H), 2.68 (s, 3H), 2.45 (s, 3H), 2.42-2.33 (m, 1H), 2.12 (s, 3H), 2.06-1.94 (m, 1H). TFA salt. | W: 300 [M + H]⁺ |
| Example 621 | 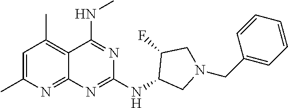 [SMILES: CNc1nc(N[C@H]2CN(Cc3ccccc3)C[C@H]2F)nc2nc(C)cc(C)c12] | Example 426, using Example 335 and appropriate aldehyde | ¹H NMR (400 MHz, Methanol-d₄) δ 7.39-7.29 (m, 4H), 7.28-7.22 (m, 1H), 6.78 (s, 1H), 5.18 (dt, J = 55.7, 4.2 Hz, 1H), 4.83-4.71 (m, 1H), 3.73 (s, 2H), 3.25-3.07 (m, 2H), 3.04 (s, 3H), 2.85 (dd, | AJ: 381 [M + H]⁺ |
| | | | J = 30.6, 12.5 Hz, 1H), 2.70 | |
| | | | (d, J = 0.9 Hz, 3H), 2.66-2.57 | |
| | | | (m, 1H), 2.45 (s, 3H). | |
| Example 622 | 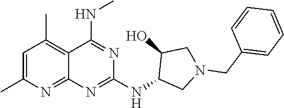 [SMILES: CNc1nc(N[C@H]2CN(Cc3ccccc3)C[C@@H]2O)nc2nc(C)cc(C)c12] | Example 426, using Example 325 and appropriate aldehyde | ¹H NMR (400 MHz, Methanol-d₄) δ 7.41-7.35 (m, 2H), 7.35-7.30 (m, 2H), 7.28- 7.23 (m, 1H), 6.80 (s, 1H), 4.33-4.12 (m, 2H), 3.75-3.61 (m, 2H), 3.15-3.07 (m, 1H), 3.04 (s, 3H), 2.99 (dd, J = | AA: 379 [M + H]⁺ |
| | | | 10.3, 6.5 Hz, 1H), 2.75-2.66 | |
| | | | (m, 3H), 2.65-2.57 (m, 1H), | |
| | | | 2.54 (dd, J = 10.4, 4.1 Hz, | |
| | | | 1H), 2.47 (s, 3H). | |
| Example 623 | 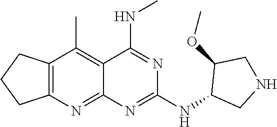 [SMILES: CNc1nc(N[C@H]2CNC[C@@H]2OC)nc2nc3c(c(C)c12)CCC3] | Example 231, using appropriate amine | ¹H NMR (400 MHz, Methanol-d₄) δ 4.54-4.43 (m, 1H), 4.00-3.89 (m, 1H), 3.44 (s, 3H), 3.42-3.35 (m, 1H), 3.28-3.21 (m, 1H), 3.07 (s, 3H), 3.03-2.88 (m, 6H), 2.66 (s, 3H), 2.20-2.09 (m, 2H). | AK: 329 [M + H]⁺ |
| Example 624 | 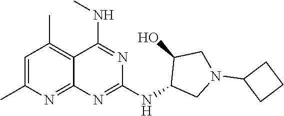 [SMILES: CNc1nc(N[C@H]2CN(C3CCC3)C[C@@H]2O)nc2nc(C)cc(C)c12] | Example 523, using Example 325 and appropriate ketone | ¹H NMR (400 MHz, Methanol-d₄) δ 6.81 (s, 1H), 4.34-4.13 (m, 2H), 3.16-3.02 (m, 5H), 2.96 (dd, J = 10.5, 6.4 Hz, 1H), 2.71 (d, J = 0.9 Hz, 3H), 2.64-2.49 (m, 2H), 2.47 (s, 3H), 2.12-1.92 (m, 4H), 1.82-1.71 (m, 2H). | AF: 343 [M + H]⁺ |
| Example 625 | 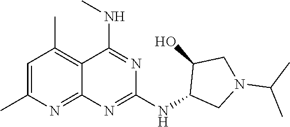 [SMILES: CNc1nc(N[C@H]2CN(C(C)C)C[C@@H]2O)nc2nc(C)cc(C)c12] | Example 523, using Example 325 and appropriate ketone | ¹H NMR (400 MHz, Methanol-d₄) δ 6.81 (s, 1H), 4.32-4.15 (m, 2H), 3.24 (t, J = 8.8 Hz, 1H), 3.18-3.09 (m, 1H), 3.06 (s, 3H), 2.77 (s, 1H), 2.73-2.66 (m, 4H), 2.64- 2.54 (m, 1H), 2.48 (s, 3H), 1.16 (t, J = 6.5 Hz, 6H). | AF: 331 [M + H]⁺ |
| Example 626 | 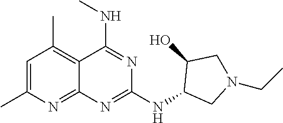 [SMILES: CCN1C[C@H](Nc2nc(NC)c3c(C)cc(C)nc3n2)[C@@H](O)C1] | Example 228, using appropriate amine | ¹H NMR (400 MHz, Methanol-d₄) δ 6.80 (s, 1H), 4.34-4.10 (m, 2H), 3.17-3.08 (m, 1H), 3.05 (s, 3H), 3.01- 2.94 (m, 1H), 2.71 (s, 3H), 2.67-2.50 (m, 4H), 2.47 (s, 3H), 1.14 (t, J = 7.2 Hz, 3H). | AN: 317 [M + H]⁺ |
| Example 627 | 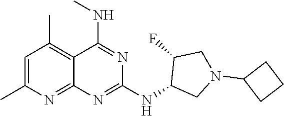 [SMILES: CNc1nc(N[C@H]2CN(C3CCC3)C[C@H]2F)nc2nc(C)cc(C)c12] | Example 523, using Example 335 and appropriate ketone | ¹H NMR (400 MHz, DMSO- d₆) δ 6.99-6.76 (m, 1H), 6.73 (s, 1H), 6.63-6.33 (m, 1H), 5.15 (dtd, J = 56.1, 4.8, 1.7 Hz, 1H), 4.57-4.36 (m, 1H), 3.22-3.01 (m, 2H), 2.99-2.82 (m, 4H), 2.75-2.52 (m, 5H), | AN: 345 [M + H]⁺ |
| | | | 2.38 (s, 3H), 2.01-1.91 (m, | |
| | | | 2H), 1.91-1.79 (m, 2H), 1.74- | |
| | | | 1.54 (m, 2H). | |
| Example 628 | 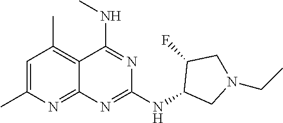 [SMILES: CCN1C[C@@H](F)[C@@H](Nc2nc(NC)c3c(C)cc(C)nc3n2)C1] | Example 228, using appropriate amine | ¹H NMR (400 MHz, DMSO- d₆) δ 6.79 (s, 1H), 6.73 (s, 1H), 6.55 (s, 1H), 5.15 (ddt, J = 56.2, 5.0, 2.5 Hz, 1H), 4.59-4.40 (m, 1H), 3.25-3.09 (m, 1H), 3.02-2.90 (m, 4H), 2.66 (s, 3H), 2.63-2.52 (m, 3H), 2.49-2.43 (m, 1H), 2.38 | AN: 319 [M + H]⁺ |
| | | | (s, 3H), 1.03 (t, J = 7.2 Hz, | |
| | | | 3H). | |
| Example 629 | 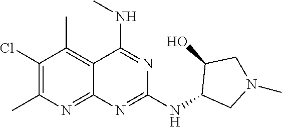 | Example 130, using Example 338 | ¹H NMR (400 MHz, DMSO- d₆) δ 7.17 (s, 1H), 7.07 (s, 1H), 5.79 (s, 1H), 4.24-3.90 (m, 2H), 2.96 (d, J = 4.3 Hz, 3H), 2.92-2.82 (m, 1H), 2.78- 2.67 (m, 4H), 2.54 (s, 3H), 2.48-2.33 (m, 2H), 2.21 (s, 3H). | AJ: 337 [M + H]⁺ |
| Example 630 | 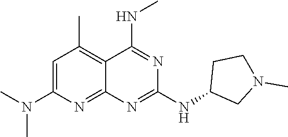 | Example 524, using dimethyl- amine | ¹H NMR (400 MHz, Methanol-d₄) δ 6.29 (s, 1H), 4.71-4.60 (m, 1H), 3.14 (s, 6H), 3.02 (s, 3H), 3.01-2.92 (m, 1H), 2.83-2.70 (m, 1H), 2.63 (s, 3H), 2.60-2.48 (m, 2H), 2.44-2.33 (m, 4H), 1.82- 1.71 (m, 1H). | W: 316 [M + H]⁺ |
| Example 631 | 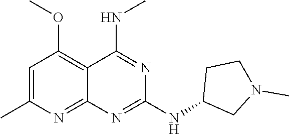 | Example 525, using methyl- amine | ¹H NMR (400 MHz, DMSO- d₆) δ 7.74 (s, 1H), 6.63 (s, 1H), 6.53 (s, 1H), 4.51-4.32 (m, 1H), 3.95 (s, 3H), 2.94 (d, J = 4.5 Hz, 3H), 2.85-2.69 (m, 1H), 2.57-2.52 (m, 1 H), 2.46- 2.41 (m, 1H), 2.39 (s, 3H), 2.35-2.29 (m, 1H), 2.23 (s, | AF: 303 [M + H]⁺ |
| | | | 3H), 2.18-2.06 (m, 1H), 1.76- | |
| | | | 1.64 (m, 1H). | |
| Example 632 | 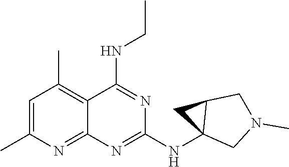 | Example 228, using appropriate amines | ¹H NMR (400 MHz, DMSO- d₆) δ 7.11 (s, 1H), 6.95-6.56 (m, 2H), 3.73-3.43 (m, 2H), 3.16-2.95 (m, 1H), 2.91-2.75 (m, 1H), 2.66 (s, 3H), 2.62- 2.51 (m, 2H), 2.37 (s, 3H), 2.24 (s, 3H), 1.57-1.47 (m, 1H), 1.30-1.16 (m, 3H), 1.15- 1.08 (m, 1H), 0.71-0.59 (m, 1H). | AB: 313 [M + H]⁺ |
| Example 633 | 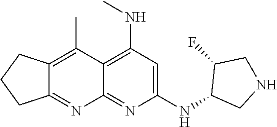 [SMILES: CNc1cc(N[C@H]2CNC[C@H]2F)nc2nc3c(c(C)c12)CCC3] | Example 425, using appropriate amine, deprotect as per Example 140 | ¹H NMR (400 MHz, Methanol-d₄) δ 5.83 (s, 1H), 5.17 (dt, J = 55.6, 4.0 Hz, 1H), 4.78-4.59 (m, 1H), 3.45- 3.32 (m, 2H), 3.22-3.07 (m, 1H), 3.03-2.93 (m, 4H), 2.89 (s, 3H), 2.79-2.67 (m, 4H), 2.21-2.09 (m, 2H). | W: 316 [M + H]⁺ |
| Example 634 | 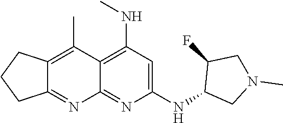 [SMILES: CNc1cc(N[C@H]2CN(C)C[C@@H]2F)nc2nc3c(c(C)c12)CCC3] | Example 425, using appropriate amine | ¹H NMR (400 MHz, Methanol-d₄) δ 5.76 (s, 1H), 5.02 (ddt, J = 52.9, 5.0, 1.6 Hz, 1H), 4.66 (dt, J = 23.9, 6.9 Hz, 1H), 3.39 (dd, J = 10.0, 7.1 Hz, 1H), 3.05-2.93 (m, 5H), 2.89 (s, 3H), 2.80 (dd, J = 11.8, 4.9 Hz, 1H), | AB: 330 [M + H]⁺ |
| | | | 2.72 (s, 3H), 2.41 (s, 3H), | |
| | | | 2.40-2.34 (m, 1H), 2.20-2.09 | |
| | | | (m, 2H). | |
| Example 635 | 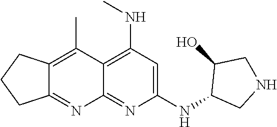 [SMILES: CNc1cc(N[C@H]2CNC[C@@H]2O)nc2nc3c(c(C)c12)CCC3] | Example 425, using appropriate amine, deprotect as per Example 140 | ¹H NMR (400 MHz, Methanol-d₄) δ 5.75 (s, 1H), 4.26-4.18 (m, 2H), 3.53-3.46 (dd, J = 12.1, 7.1 Hz, 1H), 3.24-3.17 (dd, J = 12.3, 5.3 Hz, 1H), 3.04-2.89 (m, 6H), 2.88 (s, 3H), 2.72 (s, 3H), 2.15 (p, J = 7.5 Hz, 2H). | AA: 314 [M + H]⁺ |
| Example 636 | 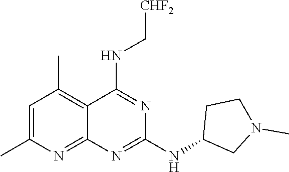 | Example 5 (Route B), using appropriate amines | ¹H NMR (400 MHz, DMSO- d₆) δ 7.14-6.83 (m, 2H), 6.73 (s, 1H), 6.58-6.12 (m, 1H), 4.43-4.31 (m, 1H), 4.00-3.75 (m, 2H), 2.78-2.69 (m, 1H), 2.66 (s, 3H), 2.60-2.53 (m, 1H), 2.45-2.35 (m, 5H), 2.24 (s, 3H), 2.21-2.11 (m, 1H), 1.77-1.64 (m, 1H). | U: 337 [M + H]⁺ |
| Example 637 | 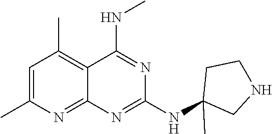 [SMILES: CNc1nc(N[C@@]2(C)CCNC2)nc2nc(C)cc(C)c12] | Example 227, using appropriate amine | | AD: 287 [M + H]⁺ |
| Example 638 | 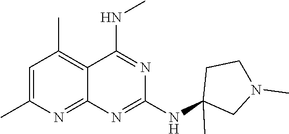 | Example 228, using appropriate amine | ¹H NMR (400 MHz, DMSO- d₆) δ 6.72 (br, 1H), 6.68 (s, 1H), 6.53 (br, 1H), 2.95 (d, J = 4.1 Hz, 3H), 2.82-2.69 (m, 2H), 2.65 (s, 3H), 2.60-2.52 (m, 1H), 2.48-2.41 (m, 1H), 2.38 (s, 3H), 2.28-2.23 (m, 1H), 2.22 (s, 3H), 1.88-1.77 (m, 1H), 1.54 (s, 3H). | AD: 301 [M + H]⁺ |
| Example 639 | 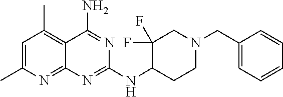 [SMILES: Cc1cc(C)c2c(N)nc(NC3CCN(Cc4ccccc4)CC3(F)F)nc2n1] | Example 131, using appropriate amine | | Library A: 399 [M + H]+ |
| Example 640 | 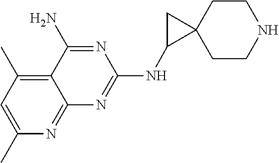 [SMILES: Cc1cc(C)c2c(N)nc(NC3CC34CCNCC4)nc2n1] | Example 131, using appropriate amine | | Library B: 299 [M + H]+ |
| Example 641 | 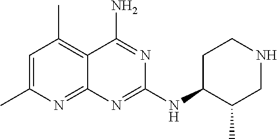 [SMILES: Cc1cc(C)c2c(N)nc(N[C@H]3CCNC[C@@H]3C)nc2n1] | Example 131, using appropriate amine | | Library B: 287 [M + H]+ |
| Example 642 | 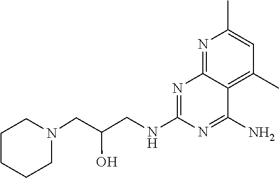 [SMILES: Cc1cc(C)c2c(N)nc(NCC(O)CN3CCCCC3)nc2n1] | Example 131, using appropriate amine | | Library E: 331 [M + H]+ |
| Example 643 | 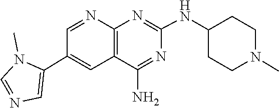 [SMILES: CN1CCC(Nc2nc(N)c3cc(-c4cncn4C)cnc3n2)CC1] | Example 49, using appropriate boronic acid or ester | | Library C: 339 [M + H]+ |
| Example 644 | 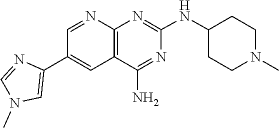 [SMILES: CN1CCC(Nc2nc(N)c3cc(-c4cn(C)cn4)cnc3n2)CC1] | Example 49, using appropriate boronic acid or ester | | Library B: 339 [M + H]+ |
| Example 645 | 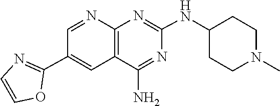 [SMILES: CN1CCC(Nc2nc(N)c3cc(-c4ncco4)cnc3n2)CC1] | Example 49, using appropriate boronic acid or ester | | Library C: 326 [M + H]+ |
| Example 646 | 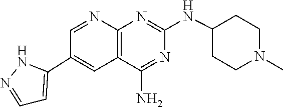 [SMILES: CN1CCC(Nc2nc(N)c3cc(-c4ccn[nH]4)cnc3n2)CC1] | Example 49, using appropriate boronic acid or ester | | Library G: 325 [M + H]+ |
| Example 647 | 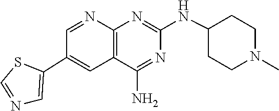 [SMILES: CN1CCC(Nc2nc(N)c3cc(-c4cncs4)cnc3n2)CC1] | Example 49, using appropriate boronic acid or ester | | Library C: 342 [M + H]+ |
| Example 648 | 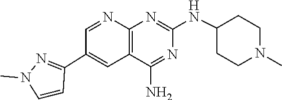 | Example 49, using appropriate boronic acid or ester | | Library H: 339 [M + H]+ |
| Example 649 | 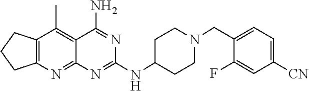 [SMILES: Cc1c2c(nc3nc(NC4CCN(Cc5ccc(C#N)cc5F)CC4)nc(N)c13)CCC2] | Example 426, using Example 577 and appropriate aldehyde | ¹H NMR (400 MHz, DMSO- d₆) δ 7.82 (dd, J = 9.9, 1.5 Hz, 1H), 7.70 (dd, J = 7.9, 1.6 Hz, 1H), 7.64 (t, J = 7.5 Hz, 1H), 6.62 (s, 2H), 6.29 (s, 1H), 3.87-3.74 (m, 1H), 3.60 (s, | AK: 432 [M + H]⁺ |
| | | | 2H), 2.91-2.74 (m, 6H), 2.57 | |
| | | | (s, 3H), 2.11 (t, J = 11.4 Hz, | |
| | | | 2H), 2.01 (p, J = 7.6 Hz, 2H), | |
| | | | 1.83 (d, J = 12.3 Hz, 2H), | |
| | | | 1.57-1.42 (m, 2H). | |
| Example 650 | 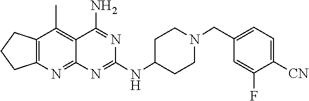 [SMILES: Cc1c2c(nc3nc(NC4CCN(Cc5ccc(C#N)c(F)c5)CC4)nc(N)c13)CCC2] | Example 426, using Example 577 and appropriate aldehyde | ¹H NMR (400 MHz, DMSO- d₆) δ 7.88 (t, J = 7.4 Hz, 1H), 7.45 (d, J = 10.5 Hz, 1H), 7.38 (dd, J = 8.0, 1.4 Hz, 1H), 6.62 (s, 2H), 6.30 (s, 1H), 3.88-3.73 (m, 1H), 3.59 (s, 2H), 2.92-2.72 (m, 6H), 2.57 | AK: 432 [M + H]⁺ |
| | | | (s, 3H), 2.16-1.96 (m, 4H), | |
| | | | 1.83 (d, J = 12.3 Hz, 2H), | |
| | | | 1.62-1.45 (m, 2H). | |
| Example 651 | 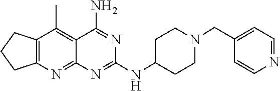 [SMILES: Cc1c2c(nc3nc(NC4CCN(Cc5ccncc5)CC4)nc(N)c13)CCC2] | Example 426, using Example 577 and appropriate aldehyde | ¹H NMR (400 MHz, DMSO- d₆) δ 8.57-8.45 (m, 2H), 7.41- 7.28 (m, 2H), 6.63 (s, 2H), 6.30 (s, 1H), 3.86-3.73 (m, 1H), 3.51 (s, 2H), 2.93-2.72 (m, 6H), 2.57 (s, 3H), 2.12- | AL: 390 [M + H]⁺ |
| | | | 1.95 (m, 4H), 1.83 (d, J = | |
| | | | 12.3 Hz, 2H), 1.61-1.47 (m, | |
| | | | 2H). | |
| Example 652 | 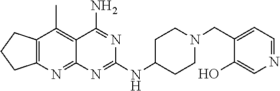 [SMILES: Cc1c2c(nc3nc(NC4CCN(Cc5ccncc5O)CC4)nc(N)c13)CCC2] | Example 426, using Example 577 and appropriate aldehyde | ¹H NMR (400 MHz, DMSO- d₆) δ 8.06 (s, 1H), 7.99 (d, J = 4.8 Hz, 1H), 7.14 (d, J = 4.8 Hz, 1H), 6.64 (s, 2H), 6.36 (s, 1H), 3.96-3.79 (m, 1H), 3.67 (s, 2H), 2.94-2.76 (m, 6H), | AY: 406 [M + H]⁺ |
| | | | 2.58 (s, 3H), 2.19 (t, J = 11.3 | |
| | | | Hz, 2H), 2.02 (p, J = 7.6 Hz, | |
| | | | 2H), 1.89 (d, J = 13.7 Hz, 2H), | |
| | | | 1.58-1.46 (m, 2H). | |
| Example 653 | 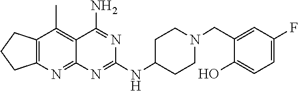 [SMILES: Cc1c2c(nc3nc(NC4CCN(Cc5cc(F)ccc5O)CC4)nc(N)c13)CCC2] | Example 426, using Example 577 and appropriate aldehyde | ¹H NMR (400 MHz, DMSO- d₆) δ 10.66 (s, 1H), 7.09-6.86 (m, 2H), 6.78-6.54 (m, 3H), 6.36 (s, 1H), 3.97-3.79 (m, 1H), 3.63 (s, 2H), 2.98-2.76 (m, 6H), 2.57 (s, 3H), 2.17 (t, | AD: 423 [M + H]⁺ |
| | | | J = 11.5 Hz, 2H), 2.02 (p, J = | |
| | | | 7.8 Hz, 2H), 1.88 (d, J = 12.6 | |
| | | | Hz, 2H), 1.53 (q, J = 11.8 Hz, | |
| | | | 2H). | |
| Example 654 | 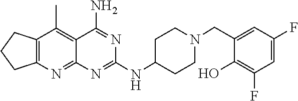 [SMILES: Cc1c2c(nc3nc(NC4CCN(Cc5cc(F)cc(F)c5O)CC4)nc(N)c13)CCC2] | Example 426, using Example 577 and appropriate aldehyde | ¹H NMR (400 MHz, DMSO- d₆) δ 7.16-7.04 (m, 1H), 6.85 (dt, J = 9.1, 2.2 Hz, 1H), 6.65 (s, 2H), 6.38 (s, 1H), 3.97-3.81 (m, 1H), 3.73 (s, 2 H), 2.98- 2.77 (m, 6H), 2.58 (s, 3H), 2.22 (t, J = 11.1 Hz, 2H), | AY: 441 [M + H]⁺ |
| | | | 2.10-1.96 (m, 2H), 1.90 (d, | |
| | | | J = 12.4 Hz, 2H), 1.63-1.44 | |
| | | | (m, 2H). | |
| Example 655 | 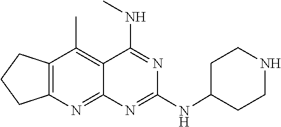 [SMILES: CNc1nc(NC2CCNCC2)nc2nc3c(c(C)c12)CCC3] | Example 231, using appropriate amine | ¹H NMR (400 MHz, Methanol-d₄) δ 4.20-4.05 (m, 1H), 3.22 (dt, J = 13.0, 4.0 Hz, 2H), 3.06 (s, 3H), 3.02- 2.84 (m, 6H), 2.65 (s, 3H), 2.23-2.04 (m, 4H), 1.66-1.51 (m, 2H). | AB: 313 [M + H]⁺ |
| Example 656 | 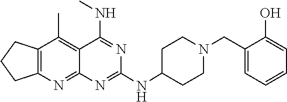 [SMILES: CNc1nc(NC2CCN(Cc3ccccc3O)CC2)nc2nc3c(c(C)c12)CCC3] | Example 426, using Example 655 and appropriate aldehyde | ¹H NMR (400 MHz, DMSO- d₆) δ 10.84 (s, 1H), 7.16-7.03 (m, 2H), 6.85-6.64 (m, 3H), 6.53 (s, 1H), 3.97-3.80 (m, 1H), 3.66 (s, 2H), 3.03-2.78 (m, 9H), 2.58 (s, 3H), 2.17 (t, J = 11.4 Hz, 2H), 2.02 (p, J = | AJ: 419 [M + H]⁺ |
| | | | 7.6 Hz, 2H), 1.91 (d, J = 12.3 | |
| | | | Hz, 2H), 1.63-1.45 (m, 2H). | |
| Example 657 | 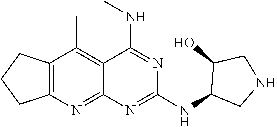 [SMILES: CNc1nc(N[C@@H]2CNC[C@@H]2O)nc2nc3c(c(C)c12)CCC3] | Example 231, using appropriate amine | ¹H NMR (400 MHz, Methanol-d₄) δ 4.52-4.42 (m, 1H), 4.36 (td, J = 5.0, 2.4 Hz, 1H), 3.38 (dd, J = 11.1, 7.5 Hz, 1H), 3.26 (dd, J = 12.4, 4.8 Hz, 1H), 3.06 (s, 3H), 3.01-2.84 (m, 6H), 2.65 (s, 3H), 2.13 (p, J = 7.6 Hz, 2H). | W: 315 [M + H]⁺ |
| Example 658 | 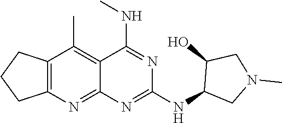 [SMILES: CNc1nc(N[C@@H]2CN(C)C[C@@H]2O)nc2nc3c(c(C)c12)CCC3] | Example 231, using appropriate amine | ¹H NMR (400 MHz, Methanol-d₄) δ 4.62-4.52 (m, 1H), 4.44-4.38 (m, 1H), 3.20- 3.09 (m, 2H), 3.06 (s, 3H), 3.01-2.89 (m, 4H), 2.65 (s, 3H), 2.60-2.49 (m, 2H), 2.39 (s, 3H), 2.13 (p, J = 7.7 Hz, 2H). | Az: 329 [M + H]⁺ |
| Example 659 | 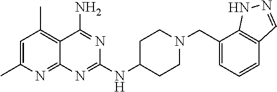 [SMILES: Cc1cc(C)c2c(N)nc(NC3CCN(Cc4cccc5cn[nH]c45)CC3)nc2n1] | Example 426, using appropriate aldehyde | ¹H NMR (400 MHz, DMSO- d₆) δ 12.92 (s, 1H), 8.07 (d, J = 1.3 Hz, 1H), 7.65 (d, J = 8.0 Hz, 1H), 7.24 (d, J = 6.9 Hz, 1H), 7.07 (d, J = 7.4 Hz, 1H), 6.88-6.54 (m, 3H), 6.34 | W: 403 [M + H]⁺ |
| | | | (s, 1H), 3.90-3.81 (m, 1H), | |
| | | | 3.77 (s, 2H), 2.89 (d, J = 10.8 | |
| | | | Hz, 2H), 2.63 (s, 3H), 2.37 (s, | |
| | | | 3H), 2.10 (t, J = 11.3 Hz, 2H), | |
| | | | 1.81 (d, J = 12.2 Hz, 2H), | |
| | | | 1.62-1.45 (m, 2H). | |
| Example 660 | 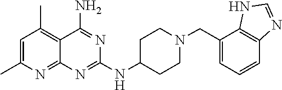 [SMILES: Cc1cc(C)c2c(N)nc(NC3CCN(Cc4cccc5nc[nH]c45)CC3)nc2n1] | Example 426, using appropriate aldehyde | ¹H NMR (400 MHz, DMSO- d₆) δ 12.53-12.15 (m, 1H), 8.17 (s, 1H), 7.63-7.37 (m, 1H), 7.21-7.02 (m, 2H), 6.89- 6.54 (m, 3H), 6.34 (s, 1H), 4.02-3.66 (m, 3H), 2.87 (d, J = | W: 403 [M + H]⁺ |
| | | | 10.9 Hz, 2H), 2.63 (s, 3H), | |
| | | | 2.37 (s, 3H), 2.10 (t, J = 11.4 | |
| | | | Hz, 2H), 1.87-1.72 (m, 2H), | |
| | | | 1.60-1.44 (m, 2H). | |
| Example 661 | 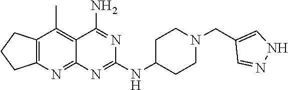 [SMILES: Cc1c2c(nc3nc(NC4CCN(Cc5cn[nH]c5)CC4)nc(N)c13)CCC2] | Example 426, using Example 577 and appropriate aldehyde | ¹H NMR (400 MHz, DMSO- d₆) δ 12.63 (s, 1H), 7.74-7.26 (m, 2H), 6.62 (s, 2H), 6.24 (s, 1H), 3.84-3.70 (m, 1H), 3.35 (s, 2H), 2.96-2.76 (m, 6H), 2.57 (s, 3H), 2.08-1.98 (m, | AE: 379 [M + H]⁺ |
| | | | 2H), 1.98-1.89 (m, 2H), 1.80 | |
| | | | (d, J = 12.0 Hz, 2H), 1.55- | |
| | | | 1.39 (m, 2H). | |
| Example 662 | 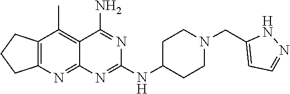 [SMILES: Cc1c2c(nc3nc(NC4CCN(Cc5ccn[nH]5)CC4)nc(N)c13)CCC2] | Example 426, using Example 577 and appropriate aldehyde | ¹H NMR (400 MHz, DMSO- d₆) δ 12.91-12.39 (m, 1H), 7.73-7.23 (m, 1H), 6.61 (s, 2H), 6.27 (s, 1H), 6.13 (s, 1H), 3.85-3.70 (m, 1H), 3.47 (s, 2H), 2.94-2.68 (m, 6H), | W: 379 [M + H]⁺ |
| | | | 2.57 (s, 3H), 2.08-1.93 (m, | |
| | | | 4H), 1.84-1.72 (m, 2H), 1.58- | |
| | | | 1.40 (m, 2H). | |
| Example 663 | 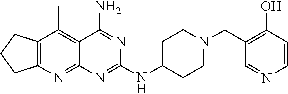 [SMILES: Cc1c2c(nc3nc(NC4CCN(Cc5cnccc5O)CC4)nc(N)c13)CCC2] | Example 426, using Example 577 and appropriate aldehyde | ¹H NMR (400 MHz, Methanol-d₄) δ 7.94 (d, J = 1.7 Hz, 1H), 7.84 (dd, J = 7.1, 1.6 Hz, 1H), 6.55 (d, J = 7.1 Hz, 1H), 4.01-3.91 (m, 1H), 3.60 (s, 2H), 3.06-2.91 (m, 6H), | AN: 406 [M + H]⁺ |
| | | | 2.67 (s, 3H), 2.45 (t, J = 11.3 | |
| | | | Hz, 2H), 2.20-2.12 (m, 2H), | |
| | | | 2.11-2.02 (m, 2H), 1.70-1.56 | |
| | | | (m, 2H). | |
| Example 664 | 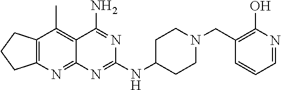 [SMILES: Cc1c2c(nc3nc(NC4CCN(Cc5cccnc5O)CC4)nc(N)c13)CCC2] | Example 426, using Example 577 and appropriate aldehyde | ¹H NMR (400 MHz, DMSO- d₆) δ 11.53 (s, 1H), 7.39 (dd, J = 6.7, 2.0 Hz, 1H), 7.26 (dd, J = 6.6, 2.1 Hz, 1H), 6.62 (s, 2H), 6.29 (s, 1H), 6.18 (t, J = 6.6 Hz, 1H), 3.87-3.74 (m, | W: 406 [M + H]⁺ |
| | | | 1H), 3.27 (s, 2H), 2.95-2.75 | |
| | | | (m, 6H), 2.57 (s, 3H), 2.13- | |
| | | | 1.94 (m, 4H), 1.89-1.77 (m, | |
| | | | 2H), 1.58-1.44 (m, 2H). | |
| Example 665 | 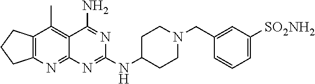 [SMILES: Cc1c2c(nc3nc(NC4CCN(Cc5cccc(S(N)(=O)=O)c5)CC4)nc(N)c13)CCC2] | Example 426, using Example 577 and appropriate aldehyde | ¹H NMR (400 MHz, DMSO- d₆) δ 7.79 (s, 1H), 7.72 (td, J = 4.6, 1.9 Hz, 1H), 7.52 (d, J = 4.6 Hz, 2H), 7.35 (s, 2H), 6.62 (s, 2H), 6.30 (s, 1H), 3.89-3.76 (m, 1H), 3.55 (s, | AA: 468 [M + H]⁺ |
| | | | 2H), 2.92-2.74 (m, 6H), 2.57 | |
| | | | (s, 3H), 2.16-1.95 (m, 4H), | |
| | | | 1.83 (d, J = 12.2 Hz, 2H), | |
| | | | 1.60-1.40 (m, 2H). | |
| Example 666 | 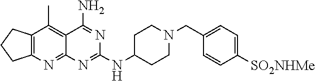 | Example 426, using Example 577 and appropriate aldehyde | ¹H NMR (400 MHz, DMSO- d₆) δ 7.73 (d, J = 8.3 Hz, 2H), 7.54 (d, J = 8.2 Hz, 2H), 7.39 (s, 1H), 6.62 (s, 2H), 6.30 (s, 1H), 3.89-3.73 (m, 1H), 3.56 | AY: 482 [M + H]⁺ |
| | | | (s, 2H), 2.93-2.74 (m, 6H), | |
| | | | 2.57 (s, 3H), 2.41 (s, 3H), | |
| | | | 2.12-1.96 (m, 4H), 1.83 (d, | |
| | | | J = 12.1 Hz, 2H), 1.59-1.46 | |
| | | | (m, 2H). | |
| Example 667 | 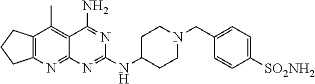 [SMILES: Cc1c2c(nc3nc(NC4CCN(Cc5ccc(S(N)(=O)=O)cc5)CC4)nc(N)c13)CCC2] | Example 426, using Example 577 and appropriate aldehyde | ¹H NMR (400 MHz, Methanol-d₄) δ 8.28 (s, 1H), 7.91 (d, J = 8.0 Hz, 2H), 7.58 (d, J = 7.9 Hz, 2H), 4.17-4.00 (m, 1H), 3.85 (s, 2H), 3.20- 2.90 (m, 6H), 2.71 (s, 3H), | AP: 468 [M + H]⁺ |
| | | | 2.59-2.39 (m, 2H), 2.20 (p, | |
| | | | J = 7.7 Hz, 2H), 2.10 (d, J = | |
| | | | 13.1 Hz, 2H), 1.84-1.62 (m, | |
| | | | 2H). Formate salt. | |
| Example 668 | 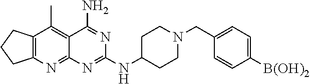 [SMILES: Cc1c2c(nc3nc(NC4CCN(Cc5ccc(B(O)O)cc5)CC4)nc(N)c13)CCC2] | Example 426, using Example 577 and appropriate aldehyde | ¹H NMR (400 MHz, DMSO- d₆) δ 8.06-7.94 (m, 2H), 7.74 (d, J = 7.7 Hz, 2H), 7.26 (d, J = 7.7 Hz, 2H), 6.63 (s, 2H), 6.28 (s, 1H), 3.88-3.72 (m, 1H), 3.46 (s, 2H), 2.91-2.72 | Az: 433 [M + H]⁺ |
| | | | (m, 6H), 2.57 (s, 3H), 2.08- | |
| | | | 1.96 (m, 4H), 1.81 (d, J = | |
| | | | 12.2 Hz, 2H), 1.50 (q, J = | |
| | | | 11.9, 11.5 Hz, 2H). | |
| Example 669 | 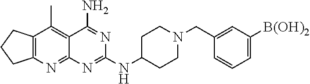 [SMILES: Cc1c2c(nc3nc(NC4CCN(Cc5cccc(B(O)O)c5)CC4)nc(N)c13)CCC2] | Example 426, using Example 577 and appropriate aldehyde | ¹H NMR (400 MHz, DMSO- d₆) δ 8.02 (s, 2H), 7.71 (s, 1H), 7.67 (dt, J = 7.1, 1.4 Hz, 1H), 7.34 (dt, J = 7.6, 1.6 Hz, 1H), 7.28 (t, J = 7.4 Hz, 1H), 6.63 (s, 2H), 6.27 (s, 1H), | AA: 433 [M + H]⁺ |
| | | | 3.89-3.72 (m, 1H), 3.45 (s, | |
| | | | 2H), 2.94-2.75 (m, 6H), 2.57 | |
| | | | (s, 3H), 2.06-1.93 (m, 4H), | |
| | | | 1.81 (d, J = 11.8 Hz, 2H), | |
| | | | 1.50 (q, J = 12.2 Hz, 2H). | |
| Example 670 | 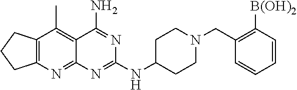 [SMILES: Cc1c2c(nc3nc(NC4CCN(Cc5ccccc5B(O)O)CC4)nc(N)c13)CCC2] | Example 426, using Example 577 and appropriate aldehyde | ¹H NMR (400 MHz, Methanol-d₄) δ 7.64 (d, J = 6.6 Hz, 1H), 7.25 (td, J = 7.0, 2.1 Hz, 1H), 7.22-7.10 (m, 2H), 4.16 (s, 3H), 3.25 (d, J = 12.7 Hz, 2H), 3.05-2.90 (m, | AB: 433 [M + H]⁺ |
| | | | 6H), 2.66 (s, 3H), 2.22 (d, J = | |
| | | | 14.2 Hz, 2H), 2.14 (p, J = 7.7 | |
| | | | Hz, 2H), 1.88-1.71 (m, 2H). | |
| Example 671 | 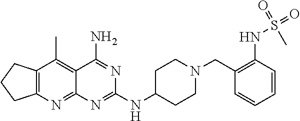 [SMILES: Cc1c2c(nc3nc(NC4CCN(Cc5ccccc5NS(C)(=O)=O)CC4)nc(N)c13)CCC2] | Example 426, using Example 577 and appropriate aldehyde | ¹H NMR (400 MHz, DMSO- d₆) δ 7.40 (dd, J = 8.1, 1.2 Hz, 1H), 7.30 (td, J = 7.7, 1.6 Hz, 1H), 7.25 (dd, J = 7.6, 1.6 Hz, 1H), 7.08 (td, J = 7.4, 1.3 Hz, 1H), 6.64 (s, 2H), 6.42 (s, 1H), 3.93-3.74 (m, 1H), 3.68 (s, 2H), 3.08 (s, 3H), 2.94- | AA: 482 [M + H]⁺ |
| | | | 2.74 (m, 6H), 2.57 (s, 3H), | |
| | | | 2.14 (t, J = 11.4 Hz, 2H), 2.02 | |
| | | | (p, J = 7.7 Hz, 2H), 1.95-1.85 | |
| | | | (m, 2H), 1.51 (q, J = 11.3 Hz, | |
| | | | 2H). | |
| Example 672 | 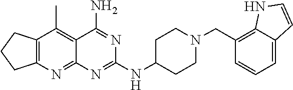 [SMILES: Cc1c2c(nc3nc(NC4CCN(Cc5cccc6cc[nH]c56)CC4)nc(N)c13)CCC2] | Example 426, using Example 577 and appropriate aldehyde | ¹H NMR (400 MHz, DMSO- d₆) δ 10.79 (s, 1H), 7.44 (d, J = 7.6 Hz, 1H), 7.31 (t, J = 2.8 Hz, 1H), 7.02-6.90 (m, 2H), 6.62 (s, 2H), 6.44-6.41 (m, 1H), 6.24 (s, 1H), 3.87- | W: 428 [M + H]⁺ |
| | | | 3.77 (m, 1H), 3.73 (s, 2H), | |
| | | | 2.95-2.77 (m, 6H), 2.57 (s, | |
| | | | 3H), 2.13-1.95 (m, 4H), 1.83 | |
| | | | (d, J = 12.0 Hz, 2H), 1.58- | |
| | | | 1.44 (m, 2H). | |
| Example 673 | 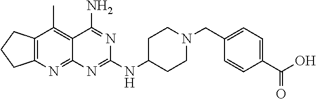 [SMILES: Cc1c2c(nc3nc(NC4CCN(Cc5ccc(C(=O)O)cc5)CC4)nc(N)c13)CCC2] | Example 426, using Example 577 and appropriate aldehyde | ¹H NMR (400 MHz, DMSO- d₆) δ 7.94-7.77 (m, 2H), 7.40 (d, J = 8.0 Hz, 2H), 6.41 (s, 2H), 5.95 (d, J = 8.1 Hz, 1H), 3.91-3.77 (m, 1H), 3.54 (s, 2H), 2.87 (q, J = 7.9 Hz, 4H), 2.82-2.75 (m, 2H), 2.58 (s, | BA: 433 [M + H]⁺ |
| | | | 3H), 2.12 (td, J = 11.4, 2.6 Hz, | |
| | | | 2H), 2.03 (p, J = 7.6 Hz, 2H), | |
| | | | 1.91-1.81 (m, 2H), 1.61-1.47 | |
| | | | (m, 2H). | |
| Example 674 | 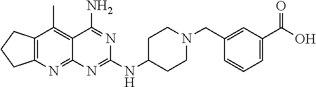 [SMILES: Cc1c2c(nc3nc(NC4CCN(Cc5cccc(C(=O)O)c5)CC4)nc(N)c13)CCC2] | Example 426, using Example 577 and appropriate aldehyde | ¹H NMR (400 MHz, DMSO- d₆) δ 7.89 (s, 1H), 7.80 (d, J = 7.7 Hz, 1H), 7.50 (d, J = 7.5 Hz, 1H), 7.40 (t, J = 7.5 Hz, 1H), 6.41 (s, 2H), 5.96 (s, 1H), 3.92-3.76 (m, 1H), 3.53 | AY: 433 [M + H]⁺ |
| | | | (s, 2H), 2.87 (q, J = 8.0 Hz, | |
| | | | 4H), 2.82-2.75 (m, 2H), 2.58 | |
| | | | (s, 3H), 2.17-2.08 (m, 2H), | |
| | | | 2.02 (q, J = 7.6 Hz, 2H), | |
| | | | 1.91-1.80 (m, 2H), 1.59-1.48 | |
| | | | (m, 2H). | |
| Example 675 | 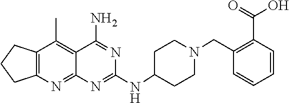 [SMILES: Cc1c2c(nc3nc(NC4CCN(Cc5ccccc5C(=O)O)CC4)nc(N)c13)CCC2] | Example 426, using Example 577 and appropriate aldehyde | ¹H NMR (400 MHz, DMSO- d₆) δ 7.91-7.80 (m, 1H), 7.49- 7.39 (m, 2H), 7.37-7.30 (m, 1H), 6.69 (s, 2H), 6.55 (s, 1H), 4.04 (s, 2H), 4.01-3.91 (m, 1H), 3.10-2.99 (m, 2H), 2.92-2.81 (m, 4H), 2.72 (t, J = | AJ: 433 [M + H]⁺ |
| | | | 11.9 Hz, 2H), 2.58 (s, 3H), | |
| | | | 2.09-1.94 (m, 4H), 1.68-1.51 | |
| | | | (m, 2H). | |
| Example 676 | 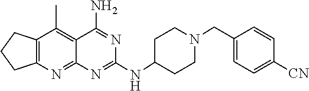 [SMILES: Cc1c2c(nc3nc(NC4CCN(Cc5ccc(C#N)cc5)CC4)nc(N)c13)CCC2] | Example 258, using appropriate amine then as per Example 426, using appropriate aldehyde | ¹H NMR (400 MHz, DMSO- d₆) δ 7.91-7.70 (m, 2H), 7.61- 7.41 (m, 2H), 6.61 (s, 1H), 6.07 (s, 2H), 5.85 (s, 1H), 3.91-3.73 (m, 1H), 3.58 (s, 2H), 3.02-2.85 (m, 4H), 2.78 | AA: 413 [M + H]⁺ |
| | | | (d, J = 11.2 Hz, 2H), 2.64 (s, | |
| | | | 3H), 2.18-1.98 (m, 4H), 1.93- | |
| | | | 1.81 (m, 2H), 1.46 (q, J = | |
| | | | 10.2, 9.7 Hz, 2H). | |
| Example 677 | 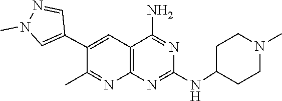 | Example 49, using Example 21 and appropriate boronic acid or ester | ¹H NMR (400 MHz, Methanol-d₄) δ 8.30 (s, 1H), 7.86 (s, 1H), 7.72 (s, 1H), 4.19-4.05 (m, 1H), 3.97 (s, 3H), 3.24 (d, J = 12.1 Hz, 2H), 2.90-2.72 (m, 2H), 2.69- 2.60 (m, 6H), 2.20 (d, J = 13.5 | C: 353 [M + H]⁺ |
| | | | Hz, 2H), 1.76 (q, J = 11.6 Hz, | |
| | | | 2H). | |
| Example 678 | 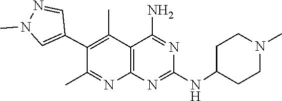 | Example 49, using Example 22 and appropriate boronic acid or ester | ¹H NMR (400 MHz, DMSO- d₆) δ 7.68 (s, 1H), 7.35 (s, 1H), 6.75 (s, 2H), 6.41 (s, 1H), 3.90 (s, 3H), 3.87-3.72 (m, 1H), 2.79 (dt, J = 12.3, 3.6 Hz, 2H), 2.43 (s, 3H), 2.23 (s, 3H), 2.20 (s, 3H), 2.03 (t, J = | C: 367 [M + H]⁺ |
| | | | 11.5 Hz, 2H), 1.87-1.78 (m, | |
| | | | 2H), 1.53 (qd, J = 12.0, 3.8 | |
| | | | Hz, 2H). | |
| Example 679 | 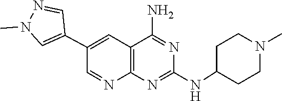 | Example 49, using Example 20 and appropriate boronic acid or ester | ¹H NMR (400 MHz, DMSO- d₆) δ 8.88 (d, J = 2.3 Hz, 1H), 8.56 (d, J = 2.5 Hz, 1H), 8.13 (s, 1H), 7.88 (s, 1H), 7.50 (s, 2H), 6.56 (s, 1H), 3.89 (s, 3H), 3.84-3.72 (m, 1H), 2.75 (d, J = 11.0 Hz, 2H), 2.16 (s, | H: 339 [M + H]⁺ |
| | | | 3H), 1.94 (t, J = 11.4 Hz, 2H), | |
| | | | 1.82 (d, J = 11.8 Hz, 2H), | |
| | | | 1.52 (qd, J = 11.8, 3.8 Hz, | |
| | | | 2H). | |
| Example 680 | 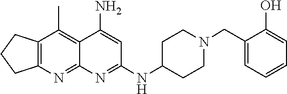 [SMILES: Cc1c2c(nc3nc(NC4CCN(Cc5ccccc5O)CC4)cc(N)c13)CCC2] | Example 258, using appropriate amine then as per Example 426, using appropriate aldehyde | ¹H NMR (400 MHz, DMSO- d₆) δ 7.15-7.04 (m, 2H), 6.77- 6.69 (m, 2H), 6.63 (s, 1H), 5.97 (s, 2H), 5.85 (s, 1H), 3.89 (s, 1H), 3.67 (s, 2H), 2.93-2.85 (m, 6H), 2.64 (s, | W: 404 [M + H]⁺ |
| | | | 3H), 2.26-2.13 (m, 2H), 2.10- | |
| | | | 1.98 (m, 2H), 1.97-1.85 (m, | |
| | | | 2H), 1.56-1.36 (m, 2H). | |
| Example 681 | 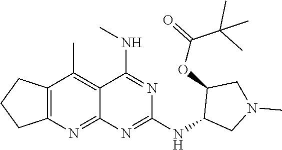 [SMILES: CNc1nc(N[C@H]2CN(C)C[C@@H]2OC(=O)C(C)(C)C)nc2nc3c(c(C)c12)CCC3] | Example 683 using appropriate acylating agent | ¹H NMR (400 MHz, DMSO- d₆) δ 8.18 (s, 1H), 7.17-6.58 (m, 2H), 5.18-5.01 (m, 1H), 4.60-4.32 (m, 1H), 3.13-3.05 (m, 1H), 2.95 (d, J = 3.9 Hz, 3H), 2.91-2.82 (m, 4H), 2.81- 2.73 (m, 1H), 2.59 (s, 3H), 2.54-2.52 (m, 1H), 2.30-2.20 (m, 4H), 2.02 (p, J = 7.6 Hz, 2H), 1.13 (s, 9H). Formate | Z: 413 [M + H]⁺ |
| | | | salt. | |
| Example 682 | 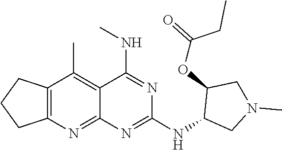 [SMILES: CCC(=O)O[C@H]1CN(C)C[C@@H]1Nc1nc(NC)c2c(C)c3c(nc2n1)CCC3] | Example 683 using appropriate acylating agent | ¹H NMR (400 MHz, DMSO- d₆) δ 6.90 (d, J = 7.7 Hz, 1H), 6.78 (s, 1H), 5.09 (s, 1H), 4.53-4.40 (m, 1H), 3.10 (t, J = 8.1 Hz, 1H), 2.94 (d, J = 4.4 Hz, 3H), 2.86 (q, J = 7.3 Hz, 4H), 2.75-2.63 (m, 1H), 2.61- 2.56 (m, 4H), 2.37-2.27 (m, 2H), 2.22 (s, 3H), 2.19-2.14 (m, 1H), 2.02 (p, J = 7.5 Hz, | AE: 385 [M + H]⁺ |
| | | | 2H), 1.00 (t, J = 7.5 Hz, 3H). | |

### Example 683: (3S,4S)-1-methyl-4-((5-methyl-4-(methylamino)-7,8-dihydro-6H-cyclopenta[5,6]pyrido[2,3-d]pyrimidin-2-yl)amino)pyrrolidin-3-yl acetate

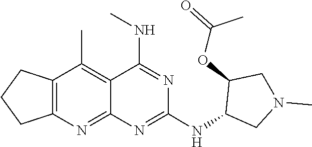 [SMILES: CNc1nc(N[C@H]2CN(C)C[C@@H]2OC(C)=O)nc2nc3c(c(C)c12)CCC3]

### Preparation 233—tert-butyl (3S,4S)-3-(((benzyloxy)carbonyl)amino)-4-hydroxypyrrolidine-1-carboxylate

To a stirred mixture of tert-butyl (3S,4S)-3-amino-4-hydroxypyrrolidine-1-carboxylate (2.00 g, 9.89 mmol) in THF (20.0 mL) and H₂O (20.0 mL) were added K₂CO₃ (4.10 g, 29.7 mmol) and CbzCl (1.82 mL, 12.9 mmol) at 0° C. The resulting mixture was stirred at room temperature for 1 h. The reaction was diluted with H₂O (20 mL). The resulting mixture was extracted with EtOAc (3×50 mL). The combined organic layers were washed with brine, dried over anhydrous Na₂SO₄. After filtration, the filtrate was concentrated under reduced pressure. The residue was purified by silica gel column chromatography, eluted with PE/EA (2:3) to afford the title compound as an off-white oil (3.00 g, 90.2%). LCMS m/z=337 [M+H]⁺

### Preparation 234—benzyl ((3S,4S)-4-hydroxypyrrolidin-3-yl)carbamate hydrochloride

A solution of tert-butyl (3S,4S)-3-(((benzyloxy)carbonyl)amino)-4-hydroxypyrrolidine-1-carboxylate (3.00 g, 8.92 mmol) in 4 M HCl in dioxane (60.0 mL) was stirred at room temperature for 3 h. The resulting mixture was concentrated under reduced pressure. The crude product (2.80 g, crude) was used in the next step directly without further purification. LCMS m/z=237 [M+H]⁺

### Preparation 235—benzyl ((3S,4S)-4-hydroxy-1-methylpyrrolidin-3-yl)carbamate

A mixture of benzyl ((3S,4S)-4-hydroxypyrrolidin-3-yl)carbamate hydrochloride (2.50 g, 9.19 mmol), Et₃N (5.10 mL, 36.8 mmol) and Paraformaldehyde (3.35 g, 110 mmol) in DCE (40.0 mL) was stirred at 40° C. for 3 h. To the above mixture was added STAB (7.80 g, 36.8 mmol) at room temperature. The resulting mixture was stirred at 40° C. for additional 1 h. The mixture was allowed to cool down to room temperature. The reaction was quenched by the addition of Water/Ice (10 mL) at 0° C. The resulting mixture was concentrated under reduced pressure. The residue was dissolved in DCM/MeOH (1:1) (100 mL). The resulting mixture was filtered, the filter cake was washed with DCM/MeOH (1:1) (3×5 mL). The filtrate was concentrated under reduced pressure. The residue was purified by Prep-HPLC^{AW} to afford the title compound as a light yellow oil (1.80 g, 80.0% for two steps). LCMS m/z=251 [M+H]⁺

### Preparation 236—(3S,4S)-4-(((benzyloxy)carbonyl)amino)-1-methylpyrrolidin-3-yl acetate

To a stirred solution of benzyl N-[(3S,4S)-4-hydroxy-1-methylpyrrolidin-3-yl]carbamate (500 mg, 2.00 mmol) in DCM (13.0 mL) was added acetic anhydride (375 μL, 3.67 mmol) at 0° C. The resulting mixture was stirred at 0° C. overnight. The resulting mixture was diluted with H₂O (15 mL). The resulting mixture was extracted with CH₂Cl₂ (3×5 mL). The combined organic layers were dried over anhydrous Na₂SO₄. After filtration, the filtrate was concentrated under reduced pressure. To afford the title compound as a light yellow solid (580 mg, crude), which was used in the next step directly without further purification. LCMS m/z=293 [M+H]⁺

### Preparation 237—(3S,4S)-4-amino-1-methylpyrrolidin-3-y acetate

A mixture of (3S,4S)-4-{[(benzyloxy)carbonyl]amino}-1-methylpyrrolidin-3-yl acetate (246 mg, 0.842 mmol) and 10% Pd/C (246 mg) in ethyl acetate (5.00 mL) was stirred under hydrogen atmosphere at room temperature for 1 h. The resulting mixture was filtered, the filter cake was washed with ethyl acetate (3×3 mL). The filtrate was concentrated under reduced pressure. This afford the title compound as an off-white oil (135 mg, crude), which was used in the next step directly without further purification. LCMS m/z=159 [M+H]⁺.

### Preparation 238—(3S,4S)-1-methyl-4-((5-methyl-4-(methylamino)-7,8-dihydro-6H-cyclopenta[5,6]pyrido[2,3-d]pyrimidin-2-yl)amino)pyrrolidin-3-yl acetate

To a stirred solution of Example 231, Preparation 118 (111 mg, 0.446 mmol) and (3S,4S)-4-amino-1-methylpyrrolidin-3-yl acetate (106 mg, 0.669 mmol) in 1,4-dioxane (5.00 mL) was added (SP-4-1)-[1,3-Bis[2,6-bis(1-ethylpropyl)phenyl]-4,5-dichloro-1,3-dihydro-2H-imidazol-2-ylidene]dichloro(2-methylpyridine)palladium (37.5 mg, 0.045 mmol) and Cs₂CO₃ (291 mg, 0.892 mmol) at room temperature. The resulting mixture was stirred under nitrogen atmosphere at 100° C. for 3 h. The mixture was allowed to cool down to room temperature. The resulting mixture was filtered, the filter cake was washed with ACN (3×5 mL). The filtrate was concentrated under reduced pressure. The residue was purified by Prep-HPLC^{BE} to afford the title compound as a light yellow solid (41.5 mg, 25.1%, ee>99%). ¹H NMR (400 MHz, DMSO-d₆) δ 6.90 (d, J=7.7 Hz, 1H), 6.78 (s, 1H), 5.07 (s, 1H), 4.53-4.42 (m, 1H), 3.11 (t, J=8.1 Hz, 1H), 2.94 (d, J=4.3 Hz, 3H), 2.91-2.82 (m, 4H), 2.73-2.60 (m, 2H), 2.59 (s, 3H), 2.22 (s, 3H), 2.19-2.11 (d, J=7.5 Hz, 1H), 2.08-1.96 (m, 5H). LCMS^{Z}m/z=371 [M+H]⁺

### Example 684: (3S,4S)-1-methyl-4-((5-methyl-4-(methylamino)-7,8-dihydro-6H-cyclopenta[5,6]pyrido[2,3-d]pyrimidin-2-yl)amino)pyrrolidin-3-yl isobutyrate

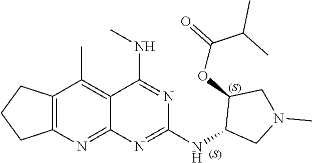

### Preparation 239—(3S,4S)-4-(((benzyloxy)carbonyl)amino)-1-methylpyrrolidin-3-yl isobutyrate

A solution of benzyl ((3S,4S)-4-hydroxy-1-methylpyrrolidin-3-yl)carbamate (400 mg, 1.60 mmol) and isobutyric anhydride (506 mg, 3.20 mmol) in DCM (8.00 mL) was stirred at room temperature overnight. The resulting mixture was diluted with water (50 mL) and extracted with CH₂Cl₂ (3×50 mL). The combined organic layers were washed with brine (20 mL), dried over anhydrous Na₂SO₄. After filtration, the filtrate was concentrated under reduced pressure to afford the title compound as a brown oil (480 mg, crude). LCMS m/z=321 [M+H]⁺.

### Preparation 240—(3S,4S)-4-amino-1-methylpyrrolidin-3-yl isobutyrate

To a solution of (3S,4S)-4-(((benzyloxy)carbonyl)amino)-1-methylpyrrolidin-3-yl isobutyrate (430 mg, 1.34 mmol) in ethyl acetate (20.0 mL) was added 10% Pd/C (1.29 g). The mixture was stirred under hydrogen atmosphere at room temperature for 2 h. The resulting was filtered through a Celite pad and washed with MeOH (3×10 mL). The combined filtrate was concentrated under reduced pressure to afford the title compound as a brown oil (210 mg, crude). LCMS m/z=187 [M+H]⁺.

### Preparation 241—(3S,4S)-1-methyl-4-((5-methyl-4-(methylamino)-7,8-dihydro-6H-cyclopenta[5,6]pyrido[2,3-d]pyrimidin-2-yl)amino)pyrrolidin-3-yl isobutyrate

A solution of Example 231, Preparation 118 (100 mg, 0.402 mmol) and (3S,4S)-4-amino-1-methylpyrrolidin-3-yl isobutyrate (112 mg, 0.603 mmol) in 1,4-dioxane (2.00 mL) was stirred at 100° C. for 3 h. The mixture was allowed to cool down to room temperature and concentrated under reduced pressure. The residue was purified by Prep-TLC (CH₂Cl₂/MeOH=20:1) to afford the title compound as a yellow solid (4.00 mg, 2.50%, ee>99%). ¹H NMR (400 MHz, DMSO-d₆) δ 7.05-6.64 (m, 2H), 5.16-5.01 (m, 1H), 4.53-4.37 (m, 1H), 3.10 (t, J=8.1 Hz, 1H), 2.94 (d, J=4.3 Hz, 3H), 2.87 (q, J=7.4 Hz, 4H), 2.79-2.71 (m, 1H), 2.59 (s, 3H), 2.57-2.53 (m, 2H), 2.28-2.15 (m, 4H), 2.07-1.96 (m, 2H), 1.15-1.01 (m, 6H). LCMS^{W} m/z=399 [M+H]⁺.

### Example 685: tert-butyl ((3S,4S)-1-methyl-4-((5-methyl-4-(methylamino)-7,8-dihydro-6H-cyclopenta[5,6]pyrido[2,3-d]pyrimidin-2-yl)amino)pyrrolidin-3-yl) carbonate

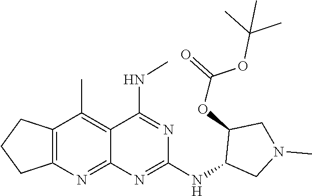 [SMILES: CNc1nc(N[C@H]2CN(C)C[C@@H]2OC(=O)OC(C)(C)C)nc2nc3c(c(C)c12)CCC3]

A solution of Example 232 (200 mg, 0.609 mmol) and di-tert-butyl dicarbonate (0.609 mmol) in DCM (3.00 mL) was stirred at room temperature for 1 h. The resulting mixture was concentrated under reduced pressure and purified to afford the title compound. ¹H NMR (400 MHz, DMSO-d₆) δ 7.75-7.42 (m, 1H), 7.30-6.83 (m, 1H), 5.06-4.91 (m, 1H), 4.57-4.41 (m, 1H), 3.21-3.09 (m, 1H), 2.99 (s, 3H), 2.95-2.83 (m, 5H), 2.81-2.68 (m, 1H), 2.62 (s, 3H), 2.46-2.38 (m, 1H), 2.34 (s, 3H), 2.05 (p, J=7.6 Hz, 2H), 1.39 (s, 9H). LCMS^{Z} m/z=429 [M+H]⁺.

### Biological Testing

### Luciferase Read-Through Assay

The read-through activity of compounds was assessed using the ADXC8 luciferase reporter cell line (generated as described in McElroy et al. PLoS Biology (2013), 11, e1001593). Active compounds promote translational read-through of the R223X stop codon in the mutated luciferase gene which leads to generation of full-length luciferase in the compound-treated cells. The amount of luciferase is measured by adding a lysis buffer containing luciferin substrate and detecting luminescence using the following protocol:

ADXC8 cells in assay media (DMEM+10% FBS) were seeded into 96-well tissue culture treated white plates (Greiner) to a density of 13,000 cells per well and incubated for 24 hours at 37° C. in an atmosphere of 5% CO₂. Compounds were solubilised in DMSO at a top concentration of 10 mM, serially diluted with assay media to 25 times final concentration and added to the plate to achieve a range of final concentrations of 100 μM to 0.045 μM in a final volume of 100 μL. Plates were incubated for a further 24 hours at 37° C. in an atmosphere of 5% CO₂. Cells treated with 200 μM G418 (with 1% DMSO) were used as a positive control.

Cell viability and firefly luciferase activity were measured in the same assay wells. CellTiter-Fluor™ reagent was prepared as per manufacturer's instructions (Promega E7120) and 20 μl of 5× CellTiter-Fluor™ reagent was added to all wells, briefly mixed by orbital shaking (300-500 rpm for −30 seconds) and the plates incubated for 30-45 minutes at 37° C. in an atmosphere of 5% C₀₂. Cell viability was measured by monitoring fluorescence on a CLARIOstar® plate reader (BMG Labtech). Cells containing 1% DMSO were used as a positive control and cells with 400 μM Doxorubicin were used as negative control to define 100 and 0% respectively. ONE-Glo™ luciferase assay buffer (Promega E7120) was prepared according to manufacturer's instructions and 100 μl of luciferase reagent was added to each well and the plate incubated at RT for 3 mins before measuring the luminescence on the CLARIOstar® plate reader (BMG Labtech). Cells treated with 200 μM G418 (with 1% DMSO) were used as a positive control and arbitrarily assigned a value of 100%. Cells treated with 1% DMSO were used as the negative control to define 0% response.

Concentration effect curves were analysed using non-linear regression using the CDD Vault from Collaborative Drug Discovery (Burlingame, CA). Where datapoints at the highest concentrations tested showed bell-shaped dose-response behaviour, they were removed from the analysis. The bottom and top of the curves were constrained to 0 and the maximum measured response respectively. From the fitted curves, the software derived C100% values (the concentration giving the same response as 200 μM G418) for each compound. The negative logarithm of C₁₀₀% is reported as pC100% (Table 1). In addition to pC100% values, the maximum fold response relative to 200 μM G418 (Fold over Control) was also captured (Table 1). A pC100% categorisation of ‘-’ in Table 1 represents examples in which a pC100% response was not reached when assayed at a concentration of 100 μM. A pC100% categorisation of ‘*’ in Table 1 represents examples in which a pC100% response was not reached when assayed at a concentration of 30 μM. For triamterene and pyrido[2,3-d]pyrimidine-2,4-diamine, a pC100% response was not reached when assayed at a concentration of 300 μM.

TABLE 1
Luciferase Assay Data for Selected Examples

| | | Fold |
|---|---|---|
| | | Over |
| Ex. | pC100% | Control |
| Triamterene | − | # |
| pyrido[2,3- | − | # |
| d]pyrimidine- | | |
| 2,4- | | |
| diamine | | |
| 1 | + | ## |
| 2 | + | ## |
| 3 | ++ | ### |
| 4 | + | ## |
| 5 | ++ | ### |
| 6 | + | ## |
| 7 | ++ | ## |
| 8 | − | # |
| 9 | ++ | ## |
| 10 | ++ | ## |
| 11 | +++ | ## |
| 12 | +++ | ## |
| 13 | * | # |
| 14 | ++ | ## |
| 15 | ++ | ## |
| 16 | +++ | ### |
| 17 | + | ## |
| 18 | + | ## |
| 19 | − | # |
| 20 | + | ## |
| 21 | ++ | ## |
| 22 | ++ | ## |
| 23 | + | ## |
| 24 | ++ | ### |
| 25 | + | ## |
| 26 | − | # |
| 27 | ++ | ## |
| 28 | − | # |
| 29 | + | ## |
| 30 | + | ## |
| 31 | ++ | ## |
| 32 | + | ## |
| 33 | + | ## |
| 34 | + | ## |
| 35 | − | # |
| 36 | + | ## |
| 37 | + | ### |
| 38 | − | # |
| 39 | + | ## |
| 40 | + | ## |
| 41 | − | # |
| 42 | − | # |
| 43 | + | ## |
| 44 | ++++ | ## |
| 45 | +++ | ## |
| 46 | +++ | ### |
| 47 | +++ | ## |
| 48 | ++ | #### |
| 49 | ++ | #### |
| 50 | ++ | #### |
| 51 | − | # |
| 52 | ++ | ## |
| 53 | ++ | ## |
| 54 | − | # |
| 55 | + | ## |
| 56 | + | ## |
| 57 | ++ | ## |
| 58 | + | ## |
| 59 | − | # |
| 60 | − | # |
| 61 | − | # |
| 62 | ++ | ## |
| 63 | ++ | ## |
| 64 | − | # |
| 65 | − | # |
| 66 | − | # |
| 67 | ++ | ## |
| 68 | + | ## |
| 69 | ++ | ## |
| 70 | + | ## |
| 71 | − | # |
| 72 | − | # |
| 73 | − | # |
| 74 | ++ | ### |
| 75 | − | # |
| 76 | + | ## |
| 77 | − | # |
| 78 | ++ | ## |
| 79 | − | # |
| 80 | + | ## |
| 81 | ++ | ### |
| 82 | ++ | #### |
| 83 | − | # |
| 84 | − | # |
| 85 | ++ | ### |
| 86 | − | # |
| 87 | − | # |
| 88 | ++ | ### |
| 89 | + | ### |
| 90 | + | ## |
| 91 | ++ | #### |
| 92 | + | ## |
| 93 | + | ## |
| 94 | + | ## |
| 95 | + | ### |
| 96 | + | ### |
| 97 | + | ### |
| 98 | − | # |
| 99 | +++ | ### |
| 100 | ++ | ## |
| 101 | ++ | ### |
| 102 | + | ## |
| 103 | ++ | ### |
| 104 | + | ### |
| 105 | + | ## |
| 106 | − | # |
| 107 | − | # |
| 108 | ++ | ### |
| 109 | + | ## |
| 110 | ++ | #### |
| 111 | + | ### |
| 112 | ++ | #### |
| 113 | + | ## |
| 114 | − | # |
| 115 | ++ | ## |
| 116 | − | # |
| 117 | − | # |
| 118 | + | ##### |
| 119 | + | ## |
| 120 | ++ | ## |
| 121 | ++ | ## |
| 122 | − | # |
| 123 | − | # |
| 124 | +++ | ## |
| 125 | − | # |
| 126 | − | # |
| 127 | +++ | ## |
| 128 | ++ | ### |
| 129 | ++ | ### |
| 130 | ++ | ## |
| 131 | − | # |
| 132 | − | # |
| 133 | − | # |
| 134 | − | # |
| 135 | − | # |
| 136 | − | # |
| 137 | + | ## |
| 138 | − | # |
| 139 | + | ## |
| 140 | ++ | ## |
| 141 | + | ## |
| 142 | ++ | ### |
| 143 | * | # |
| 144 | ++ | ## |
| 145 | ++ | ## |
| 146 | ++ | ## |
| 147 | * | # |
| 148 | + | ## |
| 149 | * | # |
| 150 | ++ | ## |
| 151 | * | # |
| 152 | + | ## |
| 153 | * | # |
| 154 | ++ | ### |
| 155 | * | # |
| 156 | ++ | ### |
| 157 | ++ | ## |
| 158 | +++ | ## |
| 159 | ++ | ### |
| 160 | − | # |
| 161 | + | ## |
| 162 | * | # |
| 163 | + | ## |
| 164 | * | # |
| 165 | * | # |
| 166 | + | ## |
| 167 | * | # |
| 168 | + | ## |
| 169 | * | # |
| 170 | + | ## |
| 171 | + | ## |
| 172 | + | ## |
| 173 | * | # |
| 174 | + | ## |
| 175 | ++ | ## |
| 176 | ++ | ## |
| 177 | +++ | ## |
| 178 | − | # |
| 179 | ++ | ## |
| 180 | ++ | ## |
| 181 | * | # |
| 182 | * | # |
| 183 | * | # |
| 184 | + | ## |
| 185 | * | # |
| 186 | + | ## |
| 187 | + | ## |
| 188 | * | # |
| 189 | * | # |
| 190 | + | ## |
| 191 | + | ## |
| 192 | * | # |
| 193 | + | ## |
| 194 | + | ## |
| 195 | * | # |
| 196 | ++ | ## |
| 197 | * | # |
| 198 | * | # |
| 199 | * | # |
| 200 | * | # |
| 201 | ++ | ## |
| 202 | + | ## |
| 203 | ++ | ## |
| 204 | ++ | ## |
| 205 | ++ | ## |
| 206 | ++ | ## |
| 207 | * | # |
| 208 | * | # |
| 209 | ++ | ## |
| 210 | ++ | ## |
| 211 | ++ | ## |
| 212 | ++ | ## |
| 213 | ++ | ## |
| 214 | * | # |
| 215 | ++ | ## |
| 216 | +++ | ### |
| 217 | ++ | ### |
| 218 | ++ | ## |
| 219 | * | # |
| 220 | ++ | ## |
| 221 | ++ | ### |
| 222 | * | # |
| 223 | + | ## |
| 224 | + | ## |
| 225 | +++ | ### |
| 226 | +++ | ## |
| 227 | +++ | ### |
| 228 | +++ | ## |
| 229 | ++++ | ## |
| 230 | +++ | ## |
| 231 | +++ | ## |
| 232 | +++ | ## |
| 233 | +++ | ## |
| 234 | +++ | #### |
| 235 | − | # |
| 236 | − | # |
| 237 | + | ## |
| 238 | * | # |
| 239 | ++ | ## |
| 240 | − | # |
| 241 | * | # |
| 242 | − | # |
| 243 | + | ## |
| 244 | − | # |
| 245 | − | # |
| 246 | − | # |
| 247 | − | # |
| 248 | − | # |
| 249 | + | ## |
| 250 | − | # |
| 251 | + | ## |
| 252 | − | # |
| 253 | ++ | ### |
| 254 | + | ## |
| 255 | +++ | ### |
| 256 | +++ | ### |
| 257 | * | # |
| 258 | +++ | ## |
| 259 | ++ | ## |
| 260 | +++ | ## |
| 261 | ++ | ## |
| 262 | + | ## |
| 263 | ++ | #### |
| 264 | ++ | ## |
| 265 | − | # |
| 266 | − | # |
| 267 | * | # |
| 268 | ++ | ## |
| 269 | ++ | ## |
| 270 | ++ | ## |
| 271 | * | # |
| 272 | +++ | ## |
| 273 | ++ | ### |
| 274 | ++ | #### |
| 275 | ++ | #### |
| 276 | + | ## |
| 277 | * | # |
| 278 | * | # |
| 279 | ++ | ## |
| 280 | ++ | #### |
| 281 | + | ## |
| 282 | * | # |
| 283 | ++ | ### |
| 284 | * | # |
| 285 | ++ | ### |
| 286 | − | # |
| 287 | − | # |
| 288 | + | ## |
| 289 | ++ | ## |
| 290 | + | ## |
| 291 | ++ | ## |
| 292 | ++ | ## |
| 293 | +++ | ### |
| 294 | +++ | ##### |
| 295 | +++ | ### |
| 296 | +++ | ### |
| 297 | + | ## |
| 298 | − | # |
| 299 | − | # |
| 300 | ++ | ## |
| 301 | ++ | ### |
| 302 | ++ | ## |
| 303 | ++ | ### |
| 304 | +++ | ### |
| 305 | * | # |
| 306 | +++ | ## |
| 307 | − | # |
| 308 | − | # |
| 309 | − | # |
| 310 | ++ | ### |
| 311 | * | # |
| 312 | + | ## |
| 313 | ++ | ## |
| 314 | * | # |
| 315 | * | # |
| 316 | * | # |
| 317 | + | ## |
| 318 | * | # |
| 319 | * | # |
| 320 | ++ | ## |
| 321 | * | # |
| 322 | +++ | ### |
| 323 | ++ | ## |
| 324 | * | # |
| 325 | ++ | ### |
| 326 | * | # |
| 327 | ++ | ## |
| 328 | ++ | ## |
| 329 | * | # |
| 330 | * | # |
| 331 | +++ | ## |
| 332 | ++ | ## |
| 333 | * | # |
| 334 | ++ | ### |
| 335 | ++ | ## |
| 336 | +++ | ## |
| 337 | +++ | ### |
| 338 | ++ | ## |
| 339 | ++ | ## |
| 340 | ++ | ## |
| 341 | ++++ | ## |
| 342 | +++ | ## |
| 343 | +++ | ## |
| 344 | ++++ | ### |
| 345 | + | ## |
| 346 | +++ | ## |
| 347 | +++ | ### |
| 348 | + | ## |
| 349 | + | ## |
| 350 | ++ | ## |
| 351 | ++ | ### |
| 352 | + | ## |
| 353 | * | # |
| 354 | * | # |
| 355 | * | # |
| 356 | * | # |
| 357 | ++ | ## |
| 358 | +++ | ## |
| 359 | + | ## |
| 360 | + | ## |
| 361 | ++ | ## |
| 362 | ++ | ## |
| 363 | +++ | ## |
| 364 | ++ | ## |
| 365 | +++ | ## |
| 366 | +++ | ## |
| 367 | ++ | ### |
| 368 | ++ | ## |
| 369 | ++ | ## |
| 370 | ++ | ## |
| 371 | + | ## |
| 372 | + | ## |
| 373 | ++ | ## |
| 374 | + | ## |
| 375 | +++ | ## |
| 376 | +++ | ### |
| 377 | ++ | ## |
| 378 | ++ | ## |
| 379 | ++ | ## |
| 380 | ++ | ## |
| 381 | ++ | ## |
| 382 | ++ | ## |
| 383 | ++ | ### |
| 384 | ++ | ## |
| 385 | +++ | ### |
| 386 | ++ | ## |
| 387 | ++ | ## |
| 388 | * | # |
| 389 | + | ## |
| 390 | ++ | ## |
| 391 | +++ | ## |
| 392 | ++ | ### |
| 393 | ++ | ## |
| 394 | ++ | ### |
| 395 | ++ | ### |
| 396 | +++ | ### |
| 397 | ++ | ## |
| 398 | * | # |
| 399 | ++ | ## |
| 400 | + | ## |
| 401 | ++ | ## |
| 402 | ++ | #### |
| 403 | ++ | #### |
| 404 | * | # |
| 405 | ++ | ## |
| 406 | ++ | #### |
| 407 | ++ | ### |
| 408 | ++ | ### |
| 409 | ++ | ## |
| 410 | * | # |
| 411 | ++ | ##### |
| 412 | ++ | ##### |
| 413 | +++ | ##### |
| 414 | ++ | ##### |
| 415 | ++ | #### |
| 416 | * | # |
| 417 | ++ | ##### |
| 418 | ++ | ### |
| 419 | +++ | ##### |
| 420 | +++ | ### |
| 421 | +++ | ## |
| 422 | +++ | ### |
| 423 | ++++ | ### |
| 424 | +++ | ## |
| 425 | +++ | ### |
| 426 | ++ | ## |
| 427 | +++ | ## |
| 428 | +++ | ## |
| 429 | +++ | ##### |
| 430 | ++ | ### |
| 431 | + | ## |
| 432 | ++ | ## |
| 433 | ++ | ## |
| 434 | ++ | ## |
| 435 | ++ | ## |
| 436 | + | ## |
| 437 | + | ## |
| 438 | * | # |
| 439 | * | # |
| 440 | * | # |
| 441 | ++ | ## |
| 442 | ++ | ### |
| 443 | ++ | ## |
| 444 | + | ## |
| 445 | ++ | ## |
| 446 | ++ | ### |
| 447 | ++ | ## |
| 448 | * | # |
| 449 | * | # |
| 450 | ++ | ## |
| 451 | * | # |
| 452 | + | ## |
| 453 | + | ## |
| 454 | ++ | ## |
| 455 | ++ | ## |
| 456 | ++ | ## |
| 457 | +++ | ## |
| 458 | + | ## |
| 459 | ++ | ## |
| 460 | ++ | ## |
| 461 | ++ | ## |
| 462 | * | # |
| 463 | ++ | ## |
| 464 | + | ## |
| 465 | ++ | ## |
| 466 | ++ | ## |
| 467 | ++ | ## |
| 468 | ++ | ## |
| 469 | + | ## |
| 470 | ++ | ## |
| 471 | ++ | ## |
| 472 | ++ | ## |
| 473 | + | ## |
| 474 | + | ## |
| 475 | + | ## |
| 476 | + | ## |
| 477 | + | ## |
| 478 | +++ | ## |
| 479 | ++ | ## |
| 480 | + | ## |
| 481 | ++ | ## |
| 482 | + | ## |
| 483 | * | # |
| 484 | + | ## |
| 485 | ++ | ## |
| 486 | +++ | ## |
| 487 | ++ | ## |
| 488 | ++ | ## |
| 489 | ++ | ## |
| 490 | + | ## |
| 491 | + | ## |
| 492 | + | ## |
| 493 | ++ | ## |
| 494 | ++ | ## |
| 495 | * | # |
| 496 | + | ## |
| 497 | + | ## |
| 498 | * | # |
| 499 | ++ | ## |
| 500 | ++ | ## |
| 501 | + | ## |
| 502 | +++ | ## |
| 503 | +++ | ## |
| 504 | ++ | ## |
| 505 | +++ | ## |
| 506 | ++ | ## |
| 507 | +++ | ## |
| 508 | ++ | ## |
| 509 | ++++ | ### |
| 510 | +++ | ## |
| 511 | ++ | ## |
| 512 | +++ | #### |
| 513 | * | # |
| 514 | ++ | ## |
| 515 | ++ | ## |
| 516 | ++++ | #### |
| 517 | ++ | ## |
| 518 | + | ## |
| 519 | * | # |
| 520 | +++ | ## |
| 521 | ++ | #### |
| 522 | * | # |
| 523 | ++ | ## |
| 524 | ++ | ## |
| 525 | + | ## |
| 526 | ++++ | ## |
| 527 | +++ | ## |
| 528 | +++ | ## |
| 529 | +++ | ## |
| 530 | +++ | ## |
| 531 | +++ | ## |
| 532 | ++++ | ## |
| 533 | + | ## |
| 534 | +++ | ## |
| 535 | +++ | ## |
| 536 | ++ | ## |
| 537 | +++ | ## |
| 538 | +++ | ## |
| 539 | +++ | ## |
| 540 | ++ | ## |
| 541 | * | # |
| 542 | ++ | ## |
| 543 | ++ | ## |
| 544 | +++ | ## |
| 545 | +++ | ### |
| 546 | ++ | ## |
| 547 | + | ## |
| 548 | +++ | ## |
| 549 | ++ | ## |
| 550 | ++ | ## |
| 551 | + | ## |
| 552 | +++ | ### |
| 553 | + | ## |
| 554 | +++ | ## |
| 555 | +++ | ## |
| 556 | +++ | ## |
| 557 | ++++ | ### |
| 558 | ++ | ## |
| 559 | ++ | ## |
| 560 | ++ | ## |
| 561 | ++ | ## |
| 562 | ++ | ## |
| 563 | +++ | #### |
| 564 | ++ | ## |
| 565 | +++ | ## |
| 566 | +++ | ## |
| 567 | +++ | ## |
| 568 | ++ | ## |
| 569 | ++ | ## |
| 570 | +++ | ## |
| 571 | ++ | ## |
| 572 | ++ | ## |
| 573 | ++ | #### |
| 574 | + | ## |
| 575 | + | ## |
| 576 | ++++ | ## |
| 577 | ++++ | ## |
| 578 | ++++ | ## |
| 579 | +++ | ## |
| 580 | ++ | ## |
| 581 | ++++ | ## |
| 582 | ++++ | ## |
| 583 | ++++ | ### |
| 584 | ++ | ### |
| 585 | ++ | ## |
| 586 | ++ | ### |
| 587 | ++ | ## |
| 588 | +++ | ### |
| 589 | ++ | ### |
| 590 | ++ | ### |
| 591 | +++ | ## |
| 592 | ++ | ## |
| 593 | ++ | ### |
| 594 | ++ | ## |
| 595 | ++ | ## |
| 596 | ++ | ## |
| 597 | ++ | ### |
| 598 | + | ## |
| 599 | ++ | ## |
| 600 | * | # |
| 601 | ++ | ### |
| 602 | ++ | ### |
| 603 | ++ | ## |
| 604 | ++ | ## |
| 605 | +++ | ## |
| 606 | ++ | ##### |
| 607 | ++ | ### |
| 608 | ++ | #### |
| 609 | ++ | ##### |
| 610 | ++ | #### |
| 611 | +++ | #### |
| 612 | + | ## |
| 613 | ++ | ### |
| 614 | ++ | ### |
| 615 | ++ | ### |
| 616 | ++ | ## |
| 617 | ++ | ## |
| 618 | ++ | ### |
| 619 | ++ | ## |
| 620 | * | # |
| 621 | + | ## |
| 622 | ++ | ## |
| 623 | +++ | ### |
| 624 | ++ | ## |
| 625 | ++ | ## |
| 626 | ++ | ## |
| 627 | + | ## |
| 628 | ++ | ### |
| 629 | ++ | ### |
| 630 | + | ## |
| 631 | ++ | ## |
| 632 | +++ | ## |
| 633 | ++ | ### |
| 634 | +++ | ## |
| 635 | ++ | ### |
| 636 | ++ | #### |
| 637 | * | # |
| 638 | * | # |
| 639 | * | # |
| 640 | * | # |
| 641 | + | ## |
| 642 | + | ## |
| 643 | − | # |
| 644 | − | # |
| 645 | − | # |
| 646 | − | # |
| 647 | + | ## |
| 648 | − | # |
| 649 | ++ | ## |
| 650 | +++ | ## |
| 651 | +++ | ## |
| 652 | +++ | ## |
| 653 | ++++ | ## |
| 654 | ++ | ## |
| 655 | ++++ | ## |
| 656 | ++++ | ## |
| 657 | ++ | ## |
| 658 | ++ | ## |
| 659 | +++ | ## |
| 660 | ++ | ## |
| 661 | ++ | ## |
| 662 | ++ | ## |
| 663 | ++ | ## |
| 664 | * | # |
| 665 | ++ | ## |
| 666 | ++ | ## |
| 667 | ++ | ## |
| 668 | ++ | ## |
| 669 | ++ | ## |
| 670 | +++ | ## |
| 671 | ++ | ## |
| 672 | ++++ | ## |
| 673 | ++ | ## |
| 674 | * | # |
| 675 | * | # |
| 676 | ++++ | ## |
| 677 | − | # |
| 678 | − | # |
| 679 | − | # |
| 680 | ++++ | ## |
| 681 | +++ | ## |
| 682 | +++ | ## |
| 683 | +++ | ## |
| 684 | +++ | ## |
| 685 | +++ | ## |

KEY
− pC100% ≤ 4.0
* pC100 ≤ 4.5
+ pC100% > 4.0 ≤ 5.0
++ pC100% > 5.0 ≤ 6.0
+++ pC100% > 6.0 ≤ 7.0
++++ pC100% > 7.0 ≤ 8.0
+++++ PC100% > 8
# Fold over Ctrl ≥ 0 ≤ 1
## Fold over Ctrl > 1 ≤ 30
### Fold over Ctrl > 30 ≤ 60
#### Fold over Ctrl > 60 ≤ 90
##### Fold over Ctrl > 90

### Read-Through of R213X Mutation in HDQ-P1 Cells by Automated Capillary Electrophoresis Western Analysis

The ability of compounds to promote read-through in cells was assessed using the HDQ-P1 cell line which contain the R213X mutation in the p53 gene. Active compounds promote translational read-through of the premature stop codon encoded by this mutation, resulting in the detection of full length p53 protein by automated capillary electrophoresis western analysis. An increase in truncated p53 may also be observed as a result of compounds suppressing nonsense mediated decay of mRNA. Changes in the protein levels of p21, a direct downstream target of p53, were also determined as evidence of functional p53 protein production.

HDQ-P1 cells (R213X) were treated with DMSO (0.3%), or test article for 24 h at various concentrations. Cells were washed once with ice cold 1×PBS and lysed in M-Per protein extraction reagent (ThermoFisher), Benzonase® Nuclease and protease inhibitor mixture (Roche) centrifuged at 4° C. for 15 min at 16 000×g, the supernatants, termed cell extracts, were collected and total proteins were quantified with Bradford protein assay kit (ThermoFisher). Briefly, mixtures of cell lysates (protein concentration 0.5 mg/mL) and the fluorescent master mix were heated at 95° C. for 5 min. The samples, chemiluminescent reagents, primary and secondary antibodies, and wash buffer were dispensed into microplates and capillary electrophoresis western analysis was carried out with the ProteinSimple JESS instrument (BioTechne) using 12-230 kDa fluorescence separation modules, DO-1 mouse anti-p53 antibody (1:100, Santa Cruz sc-126) and rabbit anti-vinculin antibody (1:200, Abcam ab129002). Signals were generated using BioTechne anti-mouse detection module and anti-rabbit NIR detection module. Similarly, p21 protein was measured with the ProteinSimple JESS instrument (BioTechne) using 12-230 kDa fluorescence separation modules, rabbit p21 (1:50, CST-2947) and anti-mouse Vinculin antibody (1:200, Anti-Vinculin, clone VIIF9, Merck MAB3574-C) and signals were generated using anti-rabbit detection module and anti-mouse NIR detection module. As an additional loading control, total protein levels were assessed with the ProteinSimple protein normalisation module. The data were acquired and analysed using the inbuilt Compass software (ProteinSimple) with the high dynamic range detection profile, which uses multiple substrate injections and exposure times. Electropherograms were converted to pseudo-blots and presented in figures for ease of visualization.

Concentration effect curves for both the production of truncated and full-length p53 were analysed using non-linear regression using the CDD Vault from Collaborative Drug Discovery (Burlingame, CA). Where datapoints at the highest concentrations tested showed bell-shaped dose-response behaviour, they were removed from the analysis. The bottom and top of the curves were constrained to 0 and the maximum measured response respectively. From the fitted curves, the software derived C100% or C50% values (the concentration giving the same or 50% of the response of 200 μM G418) for each compound. The negative logarithm of C50% or C100% is reported as pC50% NMD or pC100% RT for the production of truncated or full-length p53 respectively (Table 2).

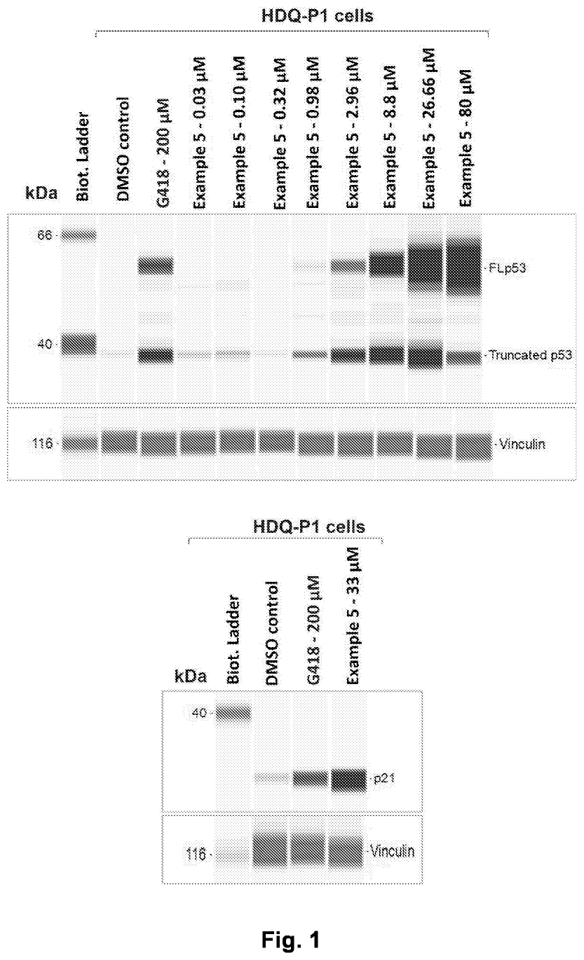

FIG. 1 shows the effect on truncated and full-length (FL) p53 expression upon treatment of HDQ-P1 cells with Example 5 (0.03-80 μM) and also the effect on p21 expression upon treatment of HDQ-P1 cells with Example 5 (33 μM) compared to DMSO (0.3%), and G418 (200 μM).

TABLE 2
HDQ-P1 p53 NMD Suppression and Read-
Through Data for Selected Examples

| Example | pC50% | pC100% |
|---|---|---|
| 5 | ++ | ++ |
| 44 | +++ | +++ |
| 45 | ++ | ++ |
| 47 | ++ | ++ |
| 48 | ++ | ++ |
| 216 | ++ | ++ |
| 225 | +++ | ++ |
| 227 | ++ | ++ |
| 228 | +++ | ++ |
| 229 | +++ | +++ |
| 230 | ++ | ++ |
| 232 | +++ | +++ |
| 233 | +++ | |
| 234 | +++ | +++ |
| 304 | ++ | ++ |
| 322 | ++ | ++ |
| 338 | ++ | |
| 343 | ++ | ++ |
| 362 | +++ | ++ |
| 363 | +++ | ++ |
| 396 | ++ | ++ |
| 420 | +++ | +++ |
| 422 | +++ | +++ |
| 423 | +++ | +++ |
| 427 | +++ | ++ |
| 428 | ++ | ++ |
| 486 | ++ | ++ |
| 516 | +++ | +++ |
| 526 | +++ | ++ |
| 529 | ++ | |
| 531 | ++ | ++ |
| 532 | +++ | ++ |
| 545 | | +++ |
| 565 | ++ | ++ |
| 576 | +++ | +++ |
| 577 | ++ | ++ |
| 578 | +++ | +++ |
| 583 | ++ | |
| 650 | ++ | ++ |
| 651 | +++ | ++ |
| 652 | ++ | |
| 653 | +++ | +++ |
| 655 | +++ | ++ |
| 656 | +++ | +++ |

KEY
+ pC50/100% > 4.0 ≤ 5.0
++ pC50/100% > 5.0 ≤ 6.0
+++ pC50/100% > 6.0 ≤ 7.0
++++ pC50/100% > 7.0

### Read-Through of Col7a 1 R578X Mutation in Transiently Transfected HEK293 Cells

The ability of compounds to promote read-through in HEK293 cells expressing a Col7a1 transcript containing an R578X mutation, which is present in recessive dystrophic epidermolysis bullosa (RDEB) patients, was determined by western blot. Active compounds promote translational read-through of the collagen 7 construct with a nonsense mutation introduced in amino acid R578X and a FLAG tag at the protein C-terminal domain, resulting in the detection of full-length Col7 protein both using endogenous Col7a1 antibody or an antibody targeting the Flag tag. An increase in truncated Col7a1 was also observed due to compounds suppressing nonsense-mediated decay of mRNA.

According to manufacturer instructions, HEK293 cells were transfected with Col7a1 R578X construct using 1 μg of plasmid DNA with Fugene HD (E2311, Promega). After overnight transfection, the media was changed for new media and cells were treated with DMSO (1%), G418 (200 μM) or test compound for 72 h at various concentrations. Cells were washed once with ice-cold 1X PBS and lysed in MRC lysis buffer (1 M Tris-HCl, pH 7.4, 0.5 M EDTA, 0.25 mM EGTA (Ethylene glycol-bis(2-aminoethyl ether)-N,N,N′,N′-tetraacetic acid), 0.5 M sodium fluoride, 0.1 M sodium pyrophosphate, 0.2 M sodium 2-glycerol 1-phosphate, 0.2 M sodium orthovanadate, 0.27 M sucrose, 10% (v/v) Triton X-100, 0.1 mM phenylmethylsulphonyl fluoride, 1 M dithiothreitol) and protease inhibitor mixture (Roche) centrifuged at 4° C. for 15 min at 16 000×g, the supernatants, termed cell extracts, were collected. Total proteins were quantified with a Bradford protein assay kit (ThermoFisher). Cell extracts were denatured by incubation in NuPAGE LDS sample buffer for 10 min at 70° C. 15 pg total protein from HEK293 cells were subjected to sodium dodecyl sulfate polyacrylamide-gel electrophoresis in 3-8% pre-cast Bis-Tris gels (ThermoFisher). Proteins were transferred onto PVDF membranes, and membranes were blocked for 1 hr at room temperature in 5% skimmed milk powder in Tris-buffered saline with 0.1% Tween® 20 Detergent (TBST) and incubated overnight with the primary mouse monoclonal antibody anti-Col7a.LH2 (152691, Cancer Tools, 1/1000), primary rabbit polyclonal antibody anti-FLAG (ab1162, Abcam, 1/5000), or primary mouse monoclonal antibody anti-Vinculin, clone VIIF9 (MAB3574-C, Merck, 1/1000). After three washes in TBST, the membranes were incubated with the secondary antibody (horseradish peroxidase-conjugated anti-mouse IgG or anti-rabbit IgG [1/10,000]) for 1 hr at room temperature. The membranes were washed three times with TBST, and chemiluminescence was detected with an ECL PRIME Western Blot detection reagent (Cytiva RPN2232, Merck) in the Li-Cor Odyssey FC imaging system. The signal was quantified using ImageJ software (NIH), and band intensity was normalised for the Vinculin signal.

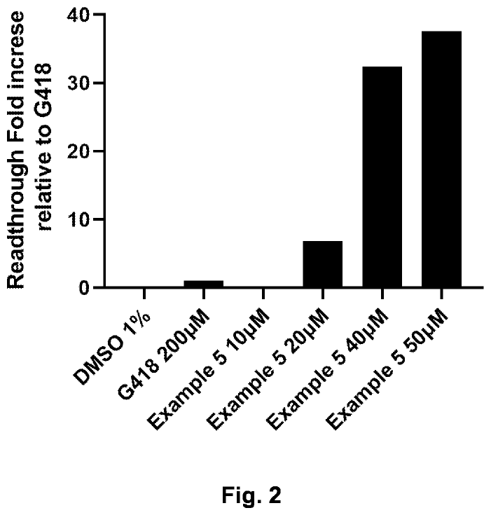

FIG. 2 shows the effect of the treatment of Col7a1 R578X transiently transfected HEK293 cells with Example 5 (10-50 μM) compared to DMSO (1%), and G418 (200 μM). These data are normalised to the effect of G418 (200 μM).

Effect on Chloride Ion Currents in a Cystic Fibrosis Patient Derived Cell Lines with Premature Stop Codons (G542X or R1162X) in the CFTR Gene

The effect of compound treatment on chloride ion currents in cystic fibrosis patient derived cell lines with premature stop codons (G542X or R1162X) in the CFTR gene was determined by electrophysiology as described previously (Valley, H. C. et al. Isogenic cell models of cystic fibrosis-causing variants in natively expressing pulmonary epithelial cells. J. Cyst. Fibros. 18, 476-483 (2018)) and briefly summarised as follows:

The parental 16HBE14o cell line, which contains two different stop mutations (G542X and R1162X) was gene-edited to provide either a G542X or an R1162X nonsense mutation into the endogenous CFTR allele (CFF-16HBEge CFTR G542X, referred to as 16HBEge G542X herein or CFF-16HBEge CFTR R1162X, referred to as 16HBEge R1162X herein) as described in Valley, H. C. et al. Isogenic cell models of cystic fibrosis-causing variants in natively expressing pulmonary epithelial cells. J. Cyst. Fibros. 18, 476-483 (2018).

16HBEge G542X or R1162X cells were seeded on HTS 24-well Transwell® Permeable Supports (Corning #3378) at 0.15×10⁶ cells/well. Cells were submerged for 7 days in MEM media (Gibco) with 10% FBS prior to the assay, with media refreshed every other day. Once ready for assay, cells were incubated with test compounds via the apical and basolateral compartments for 72 hours, with fresh medium and compound supplied after 48 hours. For equivalent current measurements, a basolateral to apical chloride ion gradient was imposed and the assay was performed, as described previously in Valley, H. C. et al. Isogenic cell models of cystic fibrosis-causing variants in natively expressing pulmonary epithelial cells. J. Cyst. Fibros. 18, 476-483 (2018). Table 3 summarises the effect of selected Examples on the transepithelial chloride conductance at the stated concentration.

TABLE 3
Effect on Chloride Ion Currents for Selected Examples

| | | Cell Line (Chloride | |
|---|---|---|---|
| | Concentration | Conductance, μA/cm²) | |
| Example | (μM) | 16HBEge G542X | 16HBEge R1162X |
| Example 5 | 20 | ++ | + |
| Example 12 | 6 | ++ | |
| Example 44 | 1 | | +++ |
| Example 47 | 20 | | +++ |
| Example 124 | 2 | | + |
| Example 216 | 10 | | +++ |
| Example 229 | 4 | +++ | |
| Example 230 | 3 | | +++ |
| Example 232 | 6 | +++ | |
| Example 263 | 20 | | ++ |
| Example 420 | 2 | +++ | |

KEY
− > 0 ≤ 0.1
+ > 0.1 ≤ 0.5
++ > 0.5 ≤ 1
+++ > 1 ≤ 5
++++ > 5 ≤ 10
+++++ > 10

### Read-Through of Col7a1 R137X Mutation in Stable Transfected HEK293 Cells

The ability of compounds to promote read-through in HEK293 cells stably expressing a Col7a1 transcript containing an R137X mutation, which is present in recessive dystrophic epidermolysis bullosa (RDEB) patients, was determined by automated capillary electrophoresis western analysis. Active compounds promote translational read-through of the collagen 7 with a nonsense mutation introduced in amino acid R137X, resulting in the detection of full-length Col7 protein using endogenous Col7a1 antibody. An increase in truncated Col7a1 was also observed due to compounds suppressing nonsense-mediated decay of mRNA.

HEK293 cells stably transfected with Col7a1 R137X were treated with DMSO (1%), G418 (200 μM) or test compound for 72 h at various concentrations. Cells were washed once with ice-cold 1X PBS and lysed in M-Per protein extraction reagent (ThermoFisher), Benzonase® Nuclease and protease inhibitor mixture (Roche) centrifuged at 4° C. for 15 min at 16 000×g, the supernatants, termed cell extracts, were collected and total proteins were quantified with Bradford protein assay kit (ThermoFisher). Briefly, mixtures of cell lysates (1 mg/ml) and the fluorescent master mix were heated at 95° C. for 5 min. The samples, chemiluminescent reagents, primary and secondary antibodies, and wash buffer were dispensed into microplates and capillary electrophoresis western analysis was carried out with the ProteinSimple JESS instrument (BioTechne) using 66-440 kDa fluorescence separation modules, primary mouse monoclonal antibody anti-Col7a.LH2 (152691, Cancer Tools, 1/75) and rabbit anti-vinculin antibody (1:100, Abcam ab129002). Signals were generated using BioTechne anti-mouse detection module and anti-rabbit NIR detection module. The data were acquired and analysed using the inbuilt Compass software (ProteinSimple) with the high dynamic range detection profile, which uses multiple substrate injections and exposure times. Electropherograms were converted to pseudo-blots and presented in figures for ease of visualization and band intensity was normalised for the Vinculin signal.

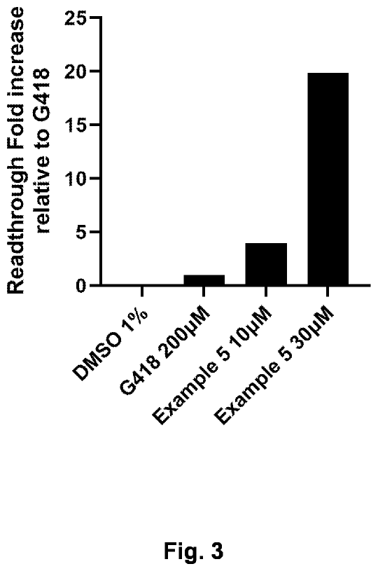

FIG. 3 shows the effect of the treatment of Col7a1 R137X stably transfected HEK293 cells with Example 5 (10-30 μM) compared to DMSO (1%), and G418 (200 μM). These data are normalised to the effect of G418 (200 μM).

Restoration of Dystrophin from Human Derived Patient Myotubes with R2905X Mutation in the DMD Gene

The ability of compounds to promote read-through in Duchenne muscular dystrophy patient myotubes with R2905X mutation in the DMD gene, was determined by automated capillary electrophoresis western analysis. Active compounds promote translational read-through of the DMD gene with a nonsense mutation introduced in amino acid R2905X, resulting in the detection of full-length dystrophin protein using dystrophin antibody on the Protein Simple JESS instrument (Bio-Techne) and compared to the levels of expression in untreated samples from wild-type immortalized human myoblasts.

AB1190 immortalized myoblasts derived from an unaffected individual and AB1023DMD11Q immortalized myoblasts derived from a Duchenne muscular dystrophy (DMD) patient with a nonsense mutation in the DMD gene (mutation stop exon 59: c.8713C>T, p. Arg2905X) from the Centre of Myologie, Paris were cultured in skeletal muscle cell growth medium (PromoCell #C23060) supplemented with FBS at 37° C. and 5% CO₂.

Cells were plated in 10 cm dishes (0.2×10⁶ cells/plate), on Matrigel coated plates. Cells were grown for 48 hours, until the cells reached 85-90% confluency. Thereafter cells were induced to differentiate into myotubes for 72 hours, prior to compound treatment. Myoblasts were differentiated into myotubes with a differentiation medium consisting of high glucose DMEM supplemented with 10 μg/mL insulin (Life Technologies #12585014). Wild-type myoblasts were used as a positive control and patient-derived DMD fibroblasts treated with DMSO was used as a negative control. DMD cells were treated daily with Gentamicin (50 μg/mL) or test compounds at different concentrations for 96 hrs.

For analysis, cells were washed once with ice cold 1×PBS and lysed in M-Per protein extraction reagent (ThermoFisher #78501), and protease inhibitor mixture (Roche) and PMSF (final concentration was 1 mM from 100 mM stock). 125-200 μL lysis buffer was used for each 10 cm plate. Lysates were incubated on ice for 15-20 min with mild shaking/vortexing of the tubes every 5 min and thereafter centrifuged at 4° C. for 15 min at maximum speed.

The supernatants, termed cell extracts, were collected and total proteins were quantified with Bradford protein assay kit (ThermoFisher). Briefly, mixtures of cell lysates (protein concentration between 0.25 mg/mL —0.5 mg/mL) and the fluorescent master mix were heated at 90° C. for 5 min. The samples, chemiluminescent reagents, primary and secondary antibodies, and wash buffer were dispensed into microplates and capillary electrophoresis western analysis was carried out with the Protein Simple JESS instrument (Bio-Techne) using 66-440 kDa fluorescence separation modules (Bio-Techne, #SM-FL005). Dystrophin antibody is diluted (1:100, Abcam #1b154168) and mouse anti-vinculin antibody (1:1000, Anti-Vinculin, clone VIIF9 #MAB3574-C). Signals were generated using BioTechne anti-rabbit detection module for dystrophin and goat anti-mouse NIR detection module for vinculin. The data were acquired and analysed using the inbuilt Compass software (ProteinSimple) with the high dynamic range detection profile, which uses multiple substrate injections and exposure times. Electropherograms were converted to pseudo-blots and presented in figures for ease of visualization. 5% of WT normal samples loading was used to calculate % of dystrophin amounts in all other DMD samples treated.

Table 4 summarises the effect of selected Examples on the restoration of full-length dystrophin after 96 hours of treatment at the stated concentration, expressed as a percentage of dystrophin from untreated, wild-type human immortalised myotubes.

TABLE 4
Effect on Dystrophin levels for Selected Examples

% Wild-Type Dystrophin

| Example | 0.5 μM | 1 μM |
|---|---|---|
| 229 | ++ | ++ |
| 232 | ++++ | +++++ |
| 427 | ++ | ++ |
| 428 | +++ | +++++ |
| 505 | ++ | ++ |
| 545 | | +++ |
| 565 | ++ | ++ |
| 576 | ++ | ++ |
| 578 | +++ | |
| 650 | +++ | ++++ |
| 652 | ++ | +++ |
| 653 | + | ++ |
| 655 | ++ | ++ |

KEY
+ ≥ 0 ≤ 0.1%
++ > 0.1 ≤ 0.5%
+++ > 0.5 ≤ 1%
++++ > 1 ≤ 1.5%
+++++ > 1.5 ≤ 2%

1. A compound of formula (I), or a tautomeric form thereof, or a pharmaceutically acceptable salt or N-oxide thereof: 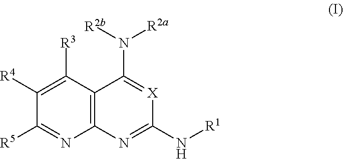
wherein
X is N or CR⁶;
R¹ is independently selected from C₀-C₆-alkylene-R^{1a}, and C₂-C₆-alkylene-R^{1b};
R^{1a} is independently selected from C₃-C₈ cycloalkyl, C₅-C₈ cycloalkenyl, 3- to 10-membered heterocycloalkyl, and 5- to 10-membered heterocycloalkenyl; wherein Ria is optionally substituted with C₀-C₆-alkylene-R^{9a} and/or from 1 to 6 R⁹ groups; wherein where Ria is heterocycloalkyl or heterocycloalkenyl, the heterocycloalkyl or heterocycloalkenyl is optionally fused to a phenyl ring, wherein the phenyl ring is optionally substituted with from 1 to 4 R¹⁰ groups;
R^{1b} is independently selected from NR^{7a}R^{8a} and OR⁷;
wherein R¹ comprises at least one nitrogen atom;
R^{2a} is independently at each occurrence selected from H and C₁-C₄ alkyl;
R^{2b} is independently at each occurrence selected from H, C₁-C₄ alkyl, C₁-C₄-haloalkyl, C₀-C₄ alkyl-R^{2c}, C₂-C₄-alkylene-R^{2d}, C(O)—C₁-C₄-alkyl, S(O)—C₁-C₄-alkyl, and S(O)₂—C₁-C₄-alkyl;
R^{2c} is independently selected from C₃-C₈ cycloalkyl, C₅-C₈ cycloalkenyl, phenyl, 3- to 8-membered heterocycloalkyl, 5- to 8-membered heterocycloalkenyl and 5-, or 6-membered heteroaryl; wherein where R^{2c} is cycloalkyl, cycloalkenyl, heterocycloalkyl or heterocycloalkenyl, R^{2c} is optionally substituted with from 1 to 4 R⁹ groups; and where R^{2c} is phenyl, or heteroaryl, R^{2c} is optionally substituted with from 1 to 5 R¹⁰ groups;
R^{2d} is independently selected from NR⁷R⁸ and OR⁷;
R³ is independently selected from H, cyano, C₁-C₄-alkylene-NR⁷R⁸, NR⁷R⁸, C₁-C₄-alkylene-OR⁷, OR⁷, SR⁷, SOR⁷, S(O)₂R⁷, S(O)₂NR⁷R⁷, CO₂R⁷, C(O)R⁷, C(O)NR⁷R⁷, C₁-C₄-alkyl, C₂-C₄-alkenyl, C₂-C₄-alkynyl, C₁-C₄-haloalkyl, NR⁷—C₀-C₄-alkylene-R^{3c}, O—C₀-C₄-alkylene-R^{3c}, and C₀-C₄-alkylene-R^{3c};
R^{3c} is independently selected from C₃-C₈ cycloalkyl, C₅-C₈ cycloalkenyl, phenyl, 3- to 10-membered heterocycloalkyl, 5- to 10-membered heterocycloalkenyl and 5-, or 6-membered heteroaryl; wherein where R^{3c} is cycloalkyl, cycloalkenyl, heterocycloalkyl or heterocycloalkenyl, R^{3c} is optionally substituted with from 1 to 4 R⁹ groups; and where R^{3c} is phenyl or heteroaryl, R^{3c} is optionally substituted with from 1 to 5 R¹⁰ groups;
R⁴ is independently selected from H, halo, cyano, NR^{1b}R⁸, OR⁷, SR⁷, SOR⁷, S(O)₂R⁷, S(O)₂NR⁷R⁷, CO₂R⁷, C(O)R⁷, C(O)NR⁷R⁷, C₁-C₄-alkyl, C₂-C₄-alkenyl, C₂-C₄-alkynyl, C₁-C₄-haloalkyl, O—C₀-C₄-alkylene-R^{4c}, and C₀-C₄-alkylene-R^{4c};
R^{4c} is independently selected from C₃-C₈ cycloalkyl, C₅-C₈ cycloalkenyl, 3- to 10-membered heterocycloalkyl, 5- to 10-membered heterocycloalkenyl and 5-membered heteroaryl; wherein where R^{4c} is cycloalkyl, cycloalkenyl, heterocycloalkyl or heterocycloalkenyl, R^{4c} is optionally substituted with from 1 to 4 R⁹ groups; and where R^{4c} is heteroaryl, R^{4c} is optionally substituted with from 1 to 5 R¹⁰ groups;
R⁵ is independently selected from H, halo, cyano, C₁-C₄-alkylene-NR⁷R⁸, NR^{7b}R⁸, C₁-C₄-alkylene-OR⁷, OR^{7b}, SR⁷, SOR⁷, S(O)₂R⁷, S(O)₂NR⁷R⁷, CO₂R⁷, C(O)R⁷, C(O)NR⁷R⁷, C₁-C₄-alkyl, C₂-C₄-alkenyl, C₂-C₄-alkynyl, C₁-C₄-haloalkyl, NR⁷—C₀-C₄-alkylene-R^{5c}, O—C₀-C₄-alkylene-R^{5c}, and C₀-C₄-alkylene-R^{5c};
R^{5c} is independently selected from C₃-C₈ cycloalkyl, C₅-C₈ cycloalkenyl, phenyl, 3- to 10-membered heterocycloalkyl, 5- to 10-membered heterocycloalkenyl and 5-, or 6-membered heteroaryl; wherein where R^{5c} is cycloalkyl, cycloalkenyl, heterocycloalkyl or heterocycloalkenyl, R^{5c} is optionally substituted with from 1 to 4 R⁹ groups; and where R^{5c} is phenyl or heteroaryl, R^{5c} is optionally substituted with from 1 to 5 R¹⁰ groups;
or R³ and R⁴ together with the carbon atoms to which they are attached form a ring selected from: phenyl, C₅-C₇-cycloalkyl, 5- to 7-membered heterocycloalkyl and 5- or 6-membered heteroaryl; wherein where the ring is cycloalkyl or heterocycloalkyl, it is optionally substituted with from 1 to 6 R⁹ groups and where the ring is phenyl or heteroaryl, it is optionally substituted with from 1 to 4 R¹⁰ groups;
or R⁴ and R⁵ together with the carbon atoms to which they are attached form a ring selected from: phenyl, C₅-C₇-cycloalkyl, 5- to 7-membered heterocycloalkyl and 5- or 6-membered heteroaryl; wherein where the ring is cycloalkyl or heterocycloalkyl, it is optionally substituted with from 1 to 6 R⁹ groups and where the ring is phenyl or heteroaryl, it is optionally substituted with from 1 to 4 R¹⁰ groups;
R⁶ is independently selected from H, halo, C₁-C₆-alkyl, and C₁-C₆-haloalkyl;
R⁷ and R^{7a} are each independently at each occurrence selected from H and C₁-C₄ alkyl;
R^{7b} is independently at each occurrence selected from C₁-C₄ alkyl;
R⁸ is independently at each occurrence selected from H, C₁-C₄ alkyl, C₁-C₄-haloalkyl and C(O)—C₁-C₄-alkyl
R^{8a} is independently at each occurrence selected from H, C₁-C₄ alkyl, C₁-C₄-haloalkyl, C(O)—C₁-C₄-alkyl and phenyl optionally substituted with from 1 to 5 R¹⁰ groups;
or R^{7a} and R^{8a}, together with the nitrogen atom to which they are attached, form a 5- to 8-membered heterocycloalkyl ring; optionally substituted with 1 to 4 R⁹ groups;
R⁹ is independently at each occurrence selected from ═O, ═S, halo, nitro, cyano, NR⁷R⁸, OR⁷, SR⁷, SOR⁷, S(O)₂R⁷, SO₂NR⁷R⁷, CO₂R⁷, C(O)R⁷, CONR⁷R⁷, C₁-C₄-alkyl, C₂-C₄-alkenyl, C₂-C₄-alkynyl, C₁-C₄-haloalkyl, C₁-C₃-alkylene-OR⁷, and C₁-C₃-alkylene-NR⁷R⁸;
R^{9a} is independently at each occurrence selected from C₃-C₈ cycloalkyl, C₅-C₈ cycloalkenyl, phenyl, 3- to 8-membered heterocycloalkyl, 5- to 8-membered heterocycloalkenyl and 5-, or 6-membered heteroaryl; wherein where R^{9a} is cycloalkyl, cycloalkenyl, heterocycloalkyl or heterocycloalkenyl, R^{9a} is optionally substituted with from 1 to 4 R⁹ groups; and where R^{9a} is phenyl, or heteroaryl, R^{9a} is optionally substituted with from 1 to 5 R¹⁰ groups;
R¹⁰ is independently at each occurrence selected from halo, nitro, cyano, NR⁷R⁸, OR⁷, SR⁷, SOR⁷, S(O)₂R⁷, SO₂NR⁷R⁷, CO₂R⁷, C(O)R⁷, CONR⁷R⁷, C₁-C₄-alkyl, C₂-C₄-alkenyl, C₂-C₄-alkynyl, C₁-C₄-haloalkyl, C₃-C₅-cycloalkyl, C₁-C₃-alkylene-NR⁷R⁸, and C₁-C₃-alkylene-OR⁷; and
wherein any of the aforementioned alkyl, alkylene, alkenyl, or cycloalkyl groups is optionally substituted, where chemically possible, by 1 to 5 substituents which are each independently at each occurrence selected from the group consisting of: C₁-C₄-alkyl, oxo, halo, nitro, cyano, NR^{a}R^{b}, OR^{a}, SR^{a}, CO₂R^{a}, C(O)R^{a}, CONR^{a}R^{a}, S(O)R^{a}, and S(O)₂R^{a}; wherein R^{a} is independently at each occurrence selected from H, and C₁-C₄-alkyl; and R^{b} is independently at each occurrence selected from H, C₁-C₄-alkyl, C(O)—C₁-C₄-alkyl and S(O)₂—C₁-C₄-alkyl.

2. A compound according to claim 1, wherein X is N.

3. A compound according to claim 1 or claim 2, wherein R^{2a} and R^{2b} are each H.

4. A compound according to any one of claims 1 to 3, wherein R¹ comprises at least one amine nitrogen.

5. A compound according to any one of claims 1 to 4, wherein R¹ is C₀-C₆-alkylene-R^{1a}.

6. A compound according to claim 5, wherein R¹ is R¹.

7. A compound according to claim 5 or claim 6, wherein R^{1a} is a 3- to 10-membered heterocycloalkyl group having a nitrogen in the ring system, wherein R^{1a} is optionally substituted with from 1 to 6 R⁹ groups.

8. A compound according to any one of claims 1 to 7, wherein R³ is H.

9. A compound according to any one of claims 1 to 7, wherein R³ is C₁-C₄-alkyl.

10. A compound according to any one of claims 1 to 9, wherein R⁴ is H.

11. A compound according to any one of claims 1 to 9, wherein R⁴ is C₁-C₄-alkyl.

12. A compound according to any one of claims 1 to 11, wherein R⁵ is R^{5c}.

13. A compound according to any one of claims 1 to 12, wherein R^{5c} is phenyl optionally substituted with from 1 to 5 R¹⁰ groups.

14. A compound according to any one of claims 1 to 12, wherein R^{5c} is 6-membered heteroaryl optionally substituted with from 1 to 5 R¹⁰ groups.

15. A compound according to any one of claims 1 to 11, wherein R⁵ is H.

16. A compound according to any one of claims 1 to 11, wherein R⁵ is C₁-C₄-alkyl.

17. A compound according to any one of claims 1 to 9, wherein R⁴ and R⁵ together with the carbon atoms to which they are attached form a C₅-C₇-cycloalkyl ring; optionally wherein the ring is substituted with from 1 to 6 R⁹ groups.

18. A compound according to claim 1, wherein the compound of formula (I) is selected from:

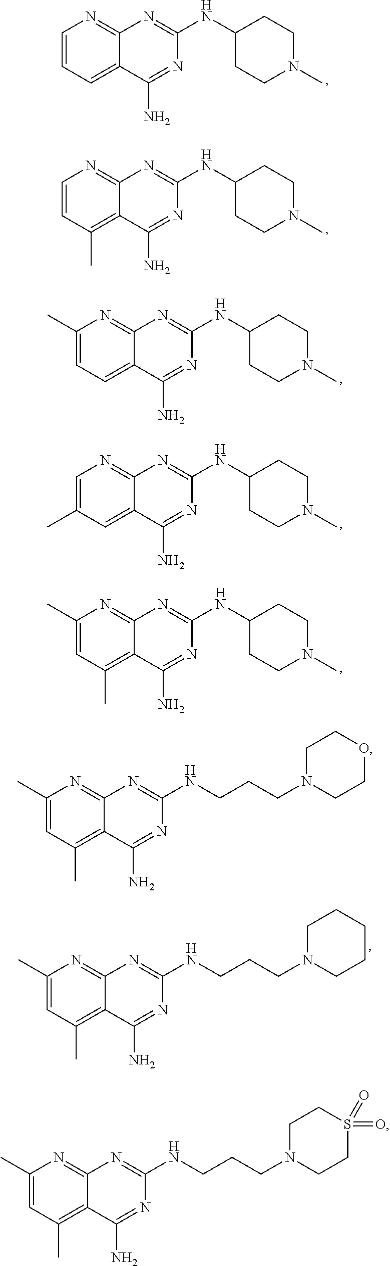

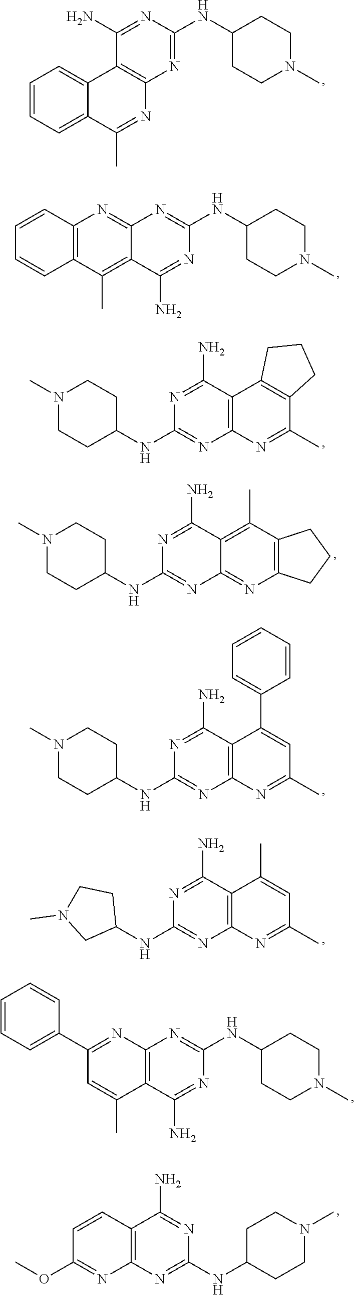

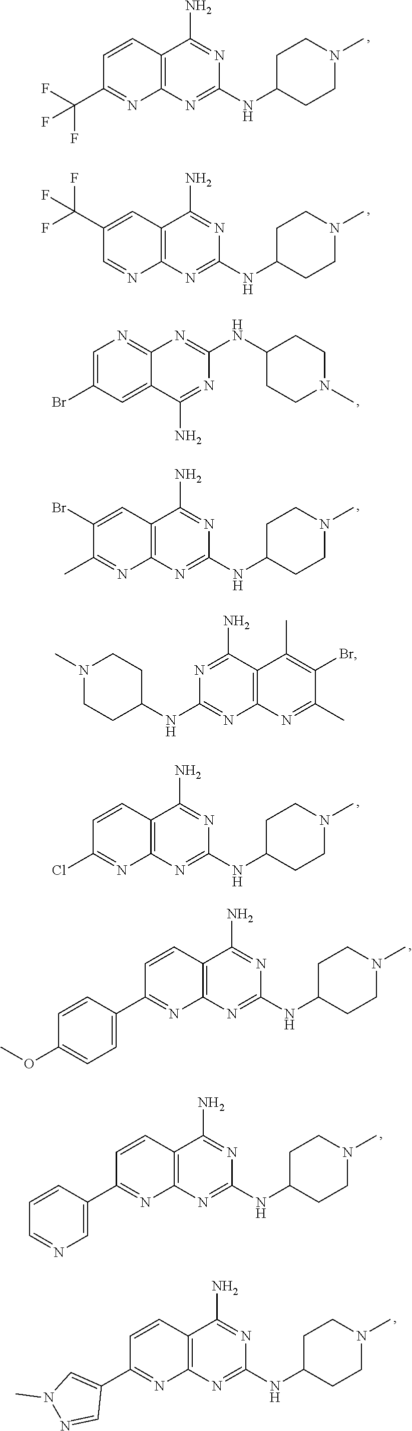

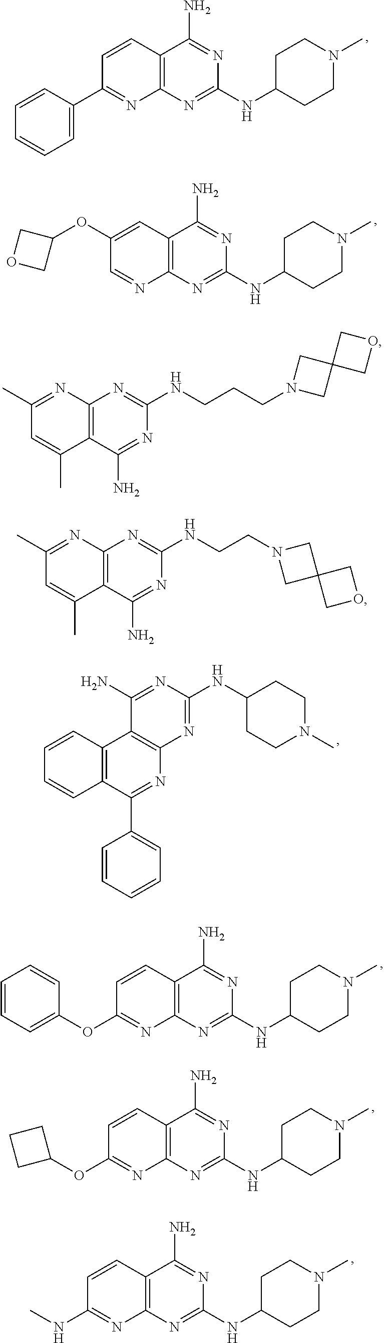

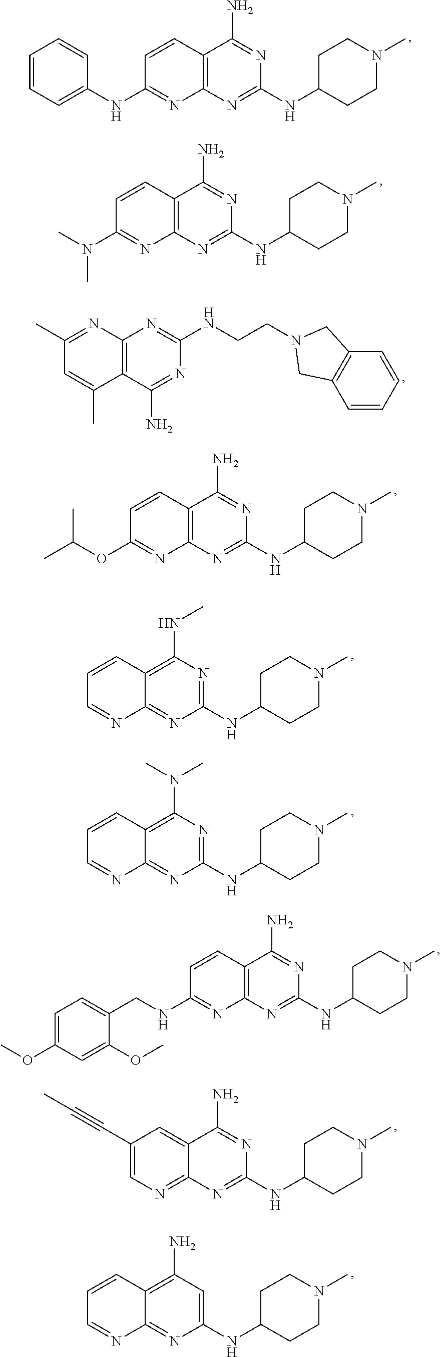

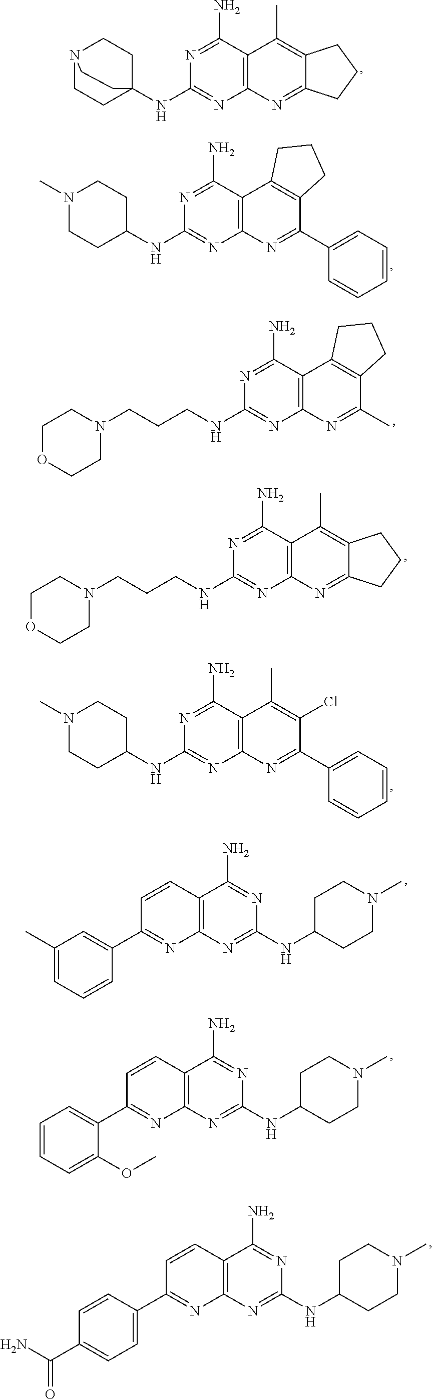

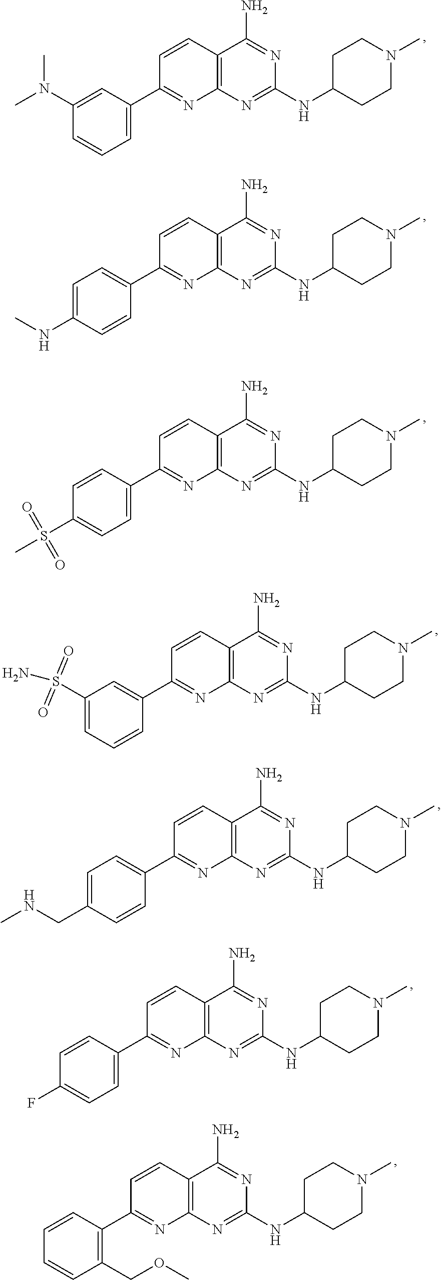

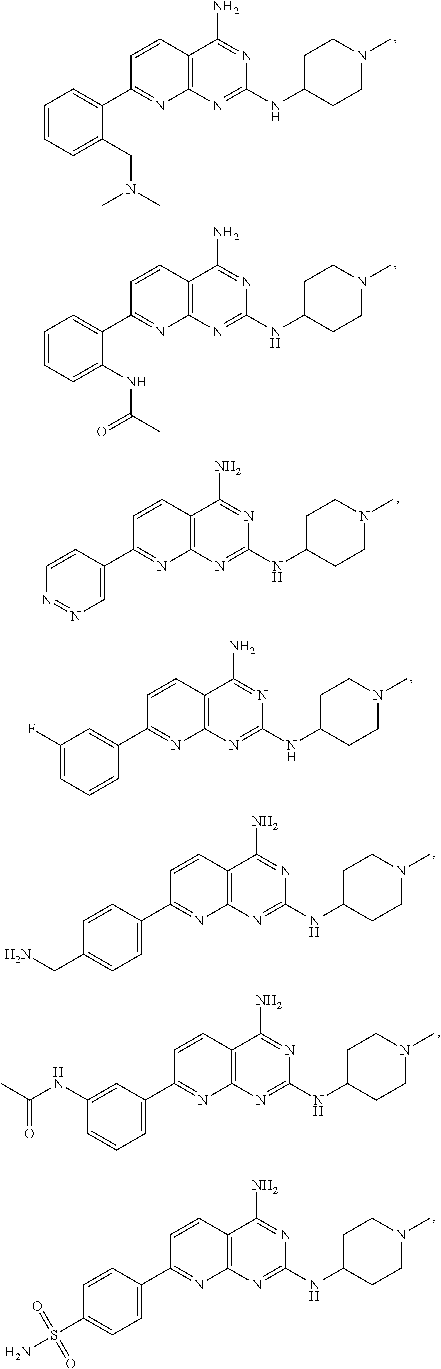

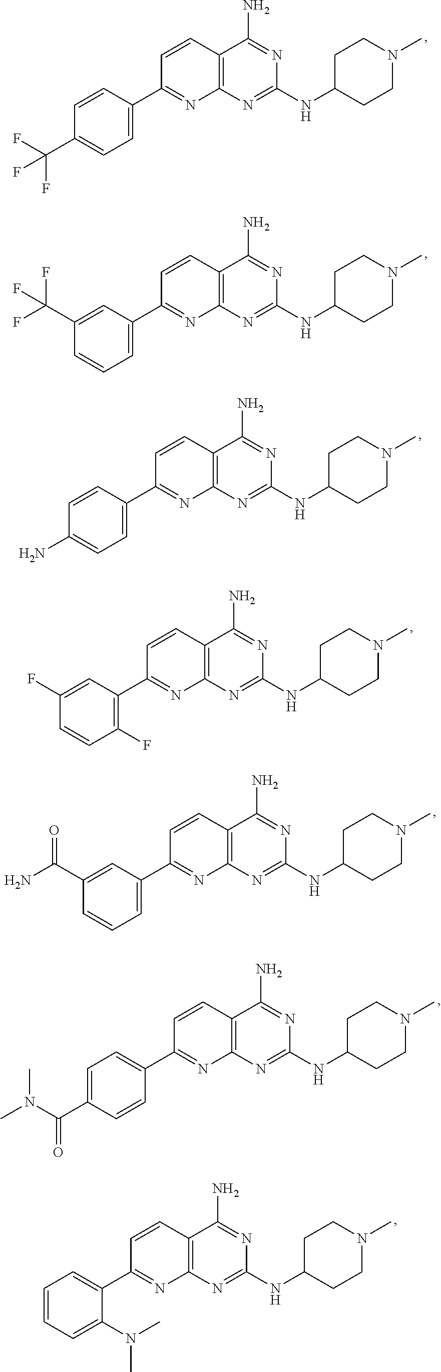

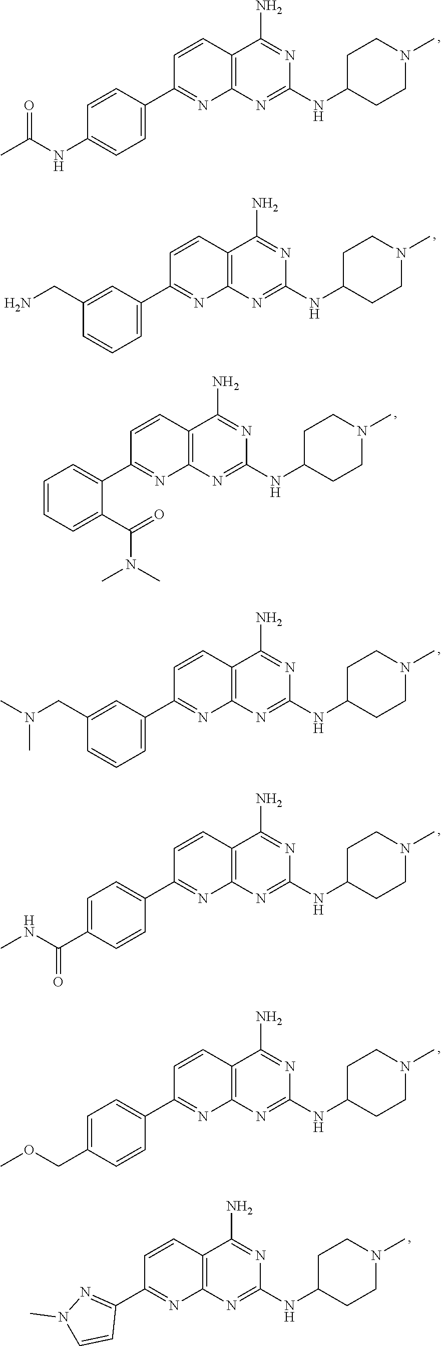

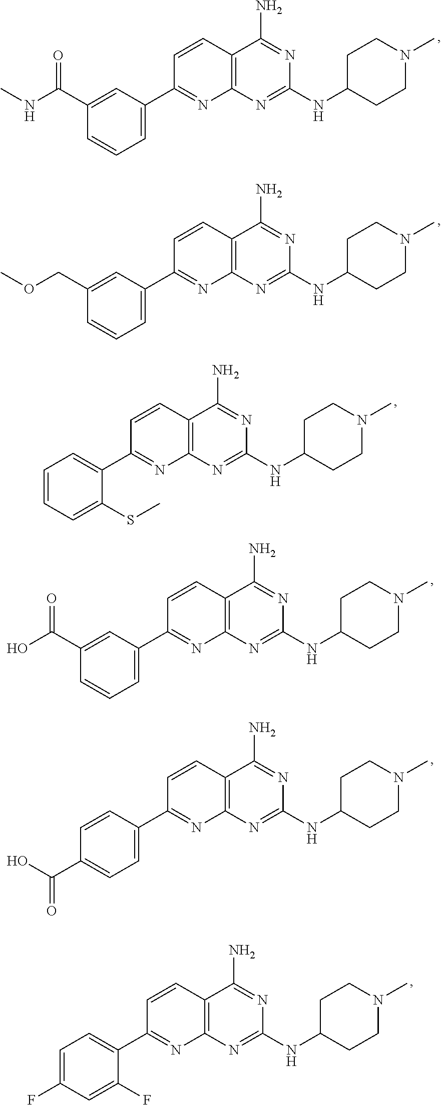

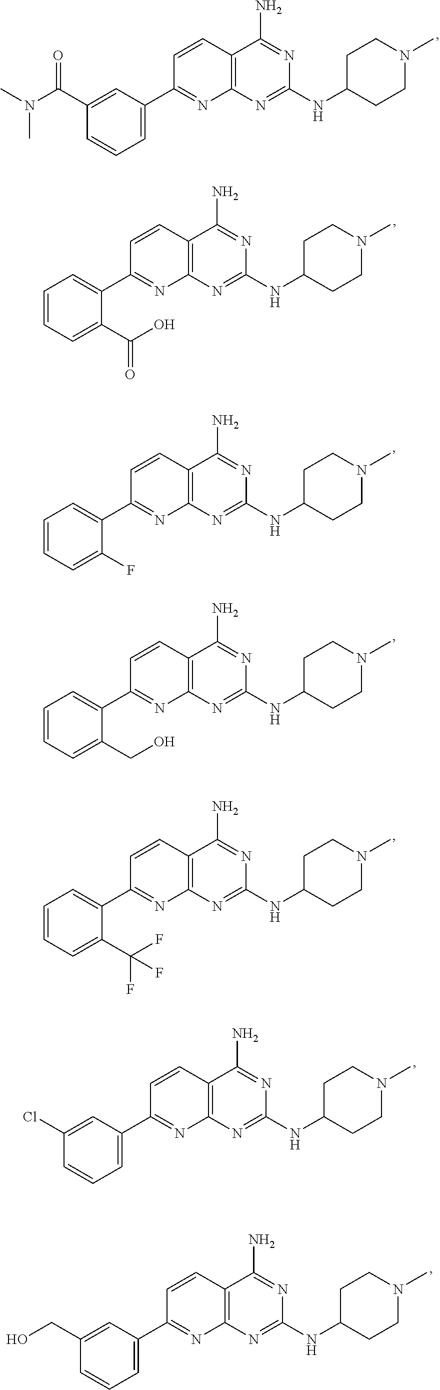

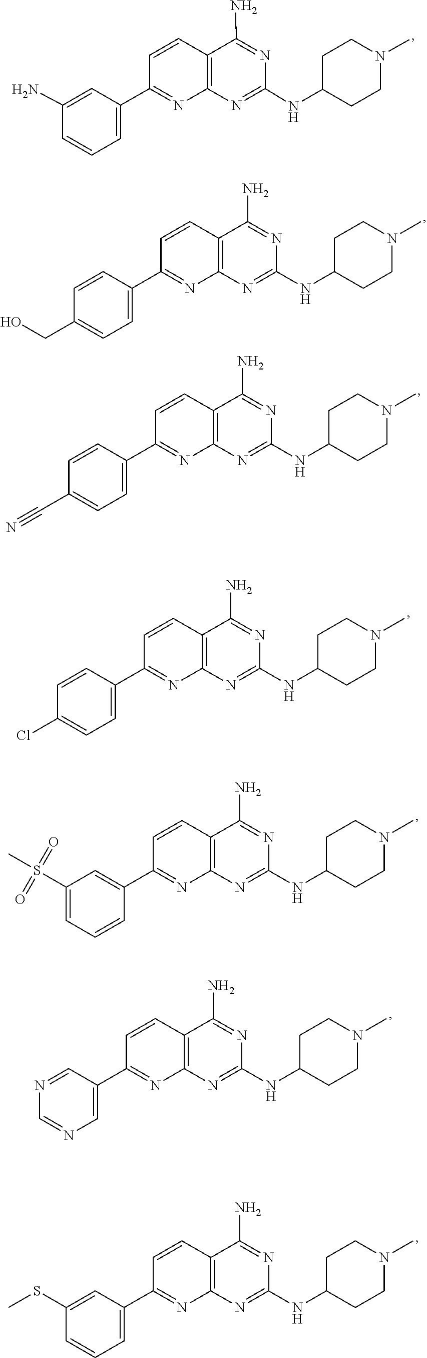

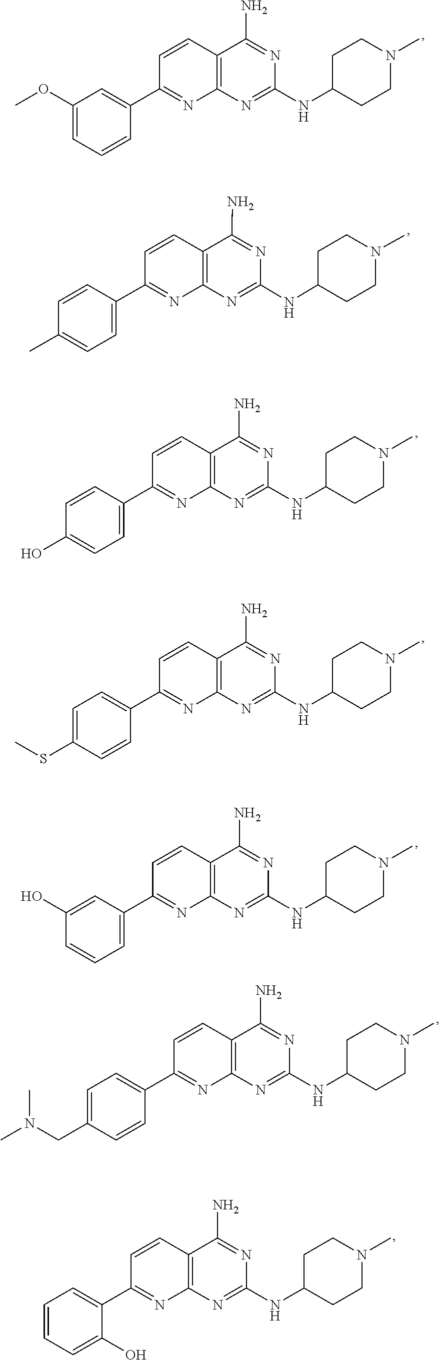

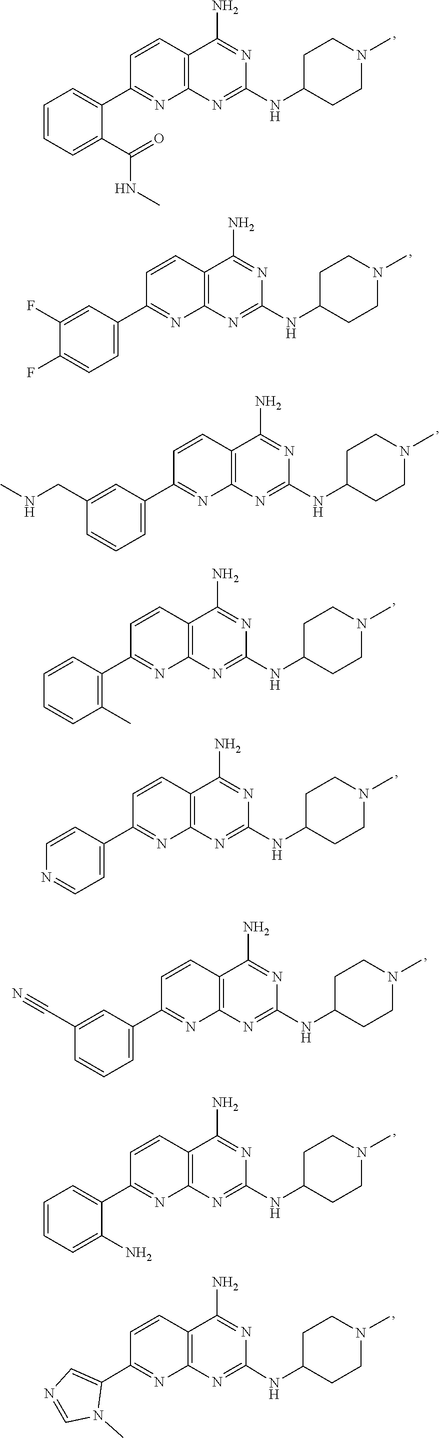

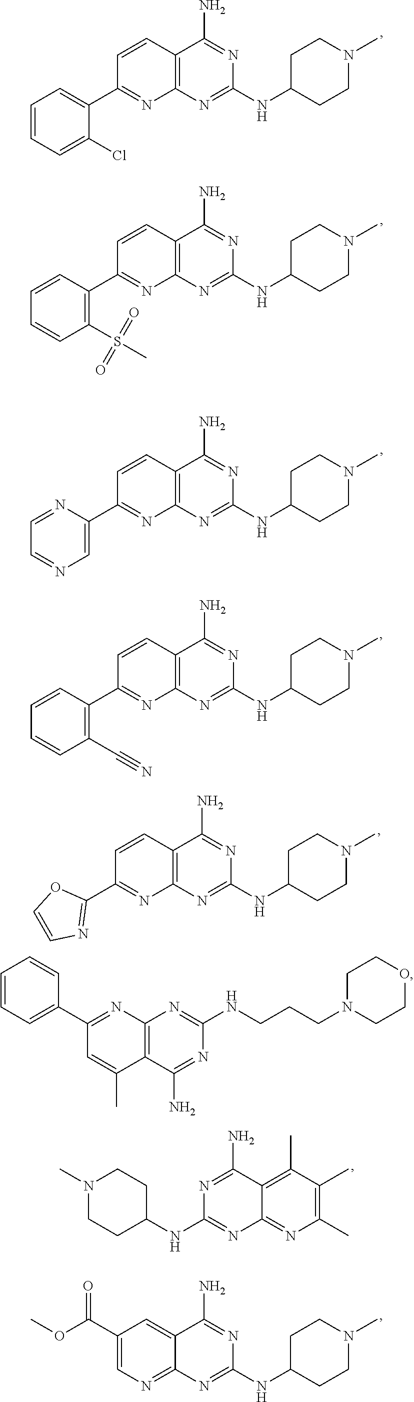

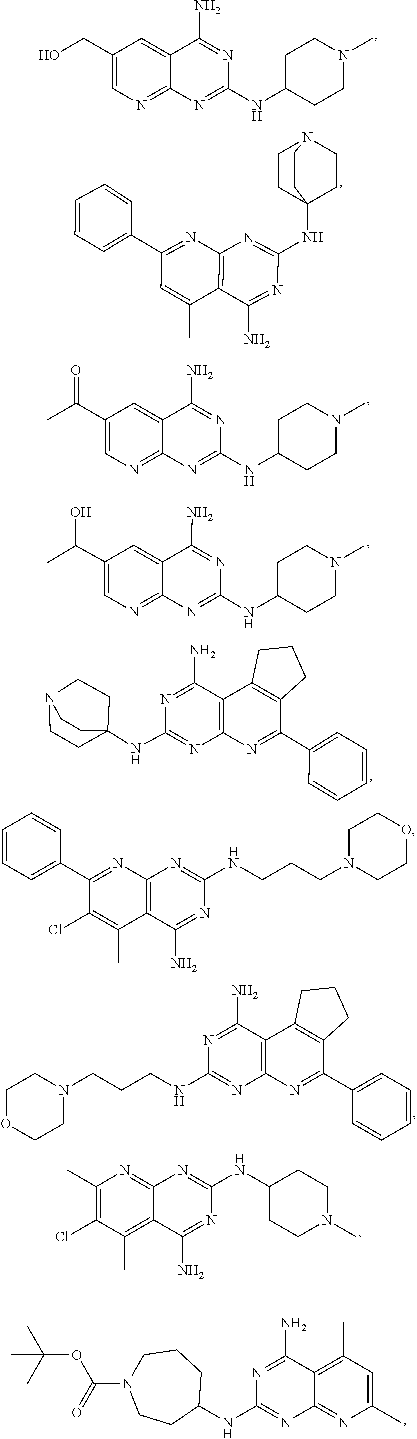

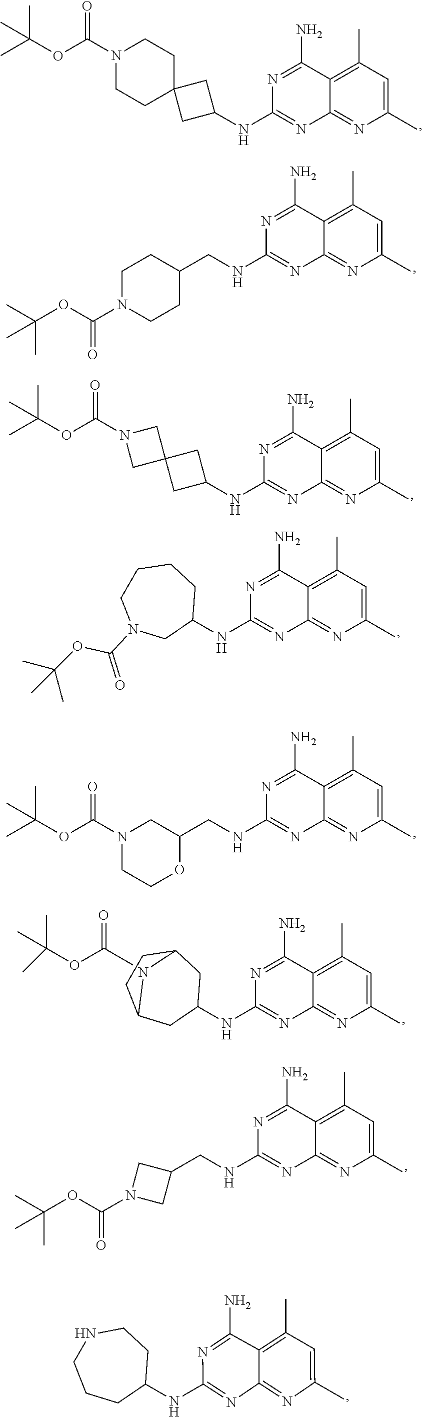

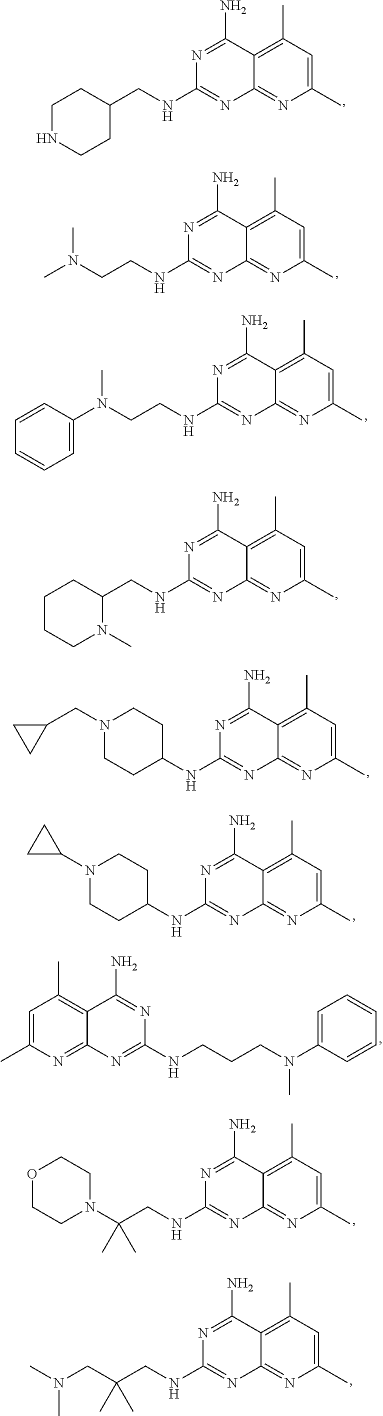

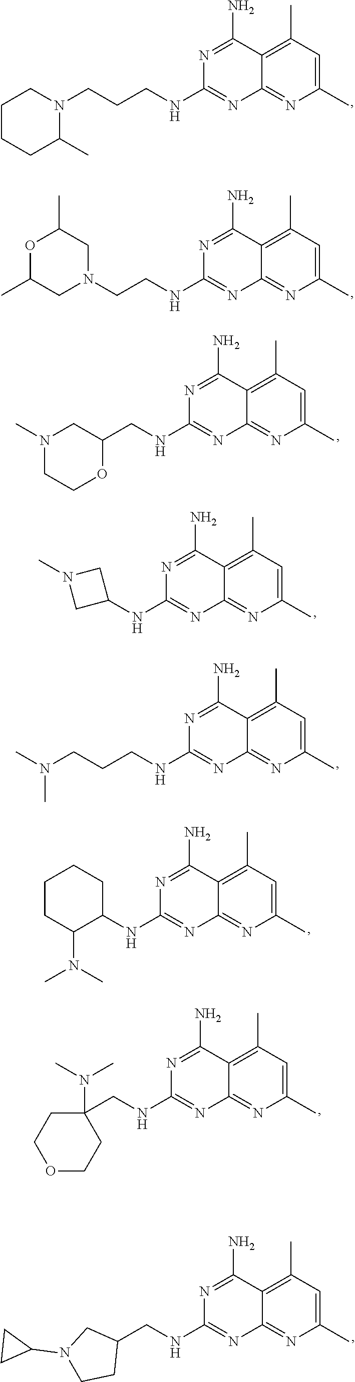

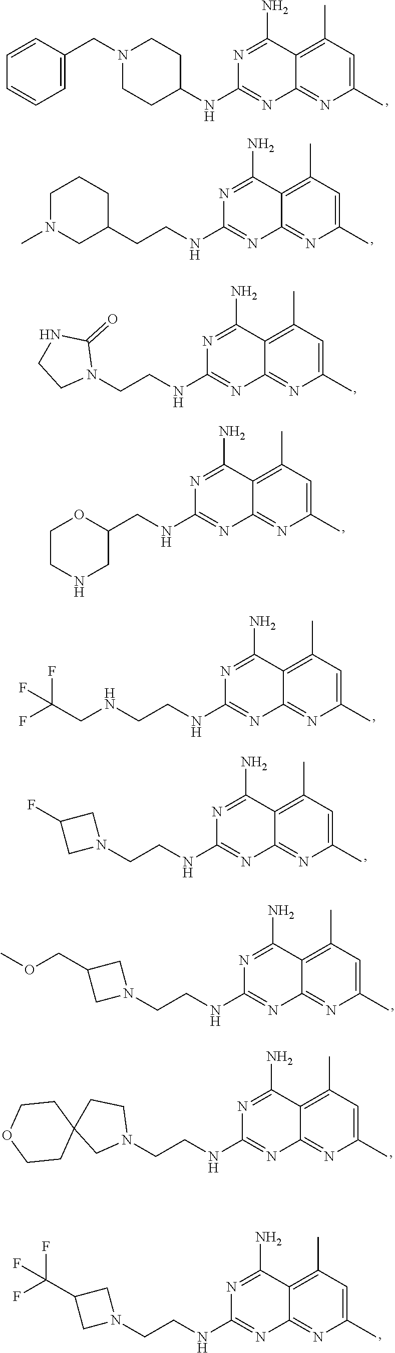

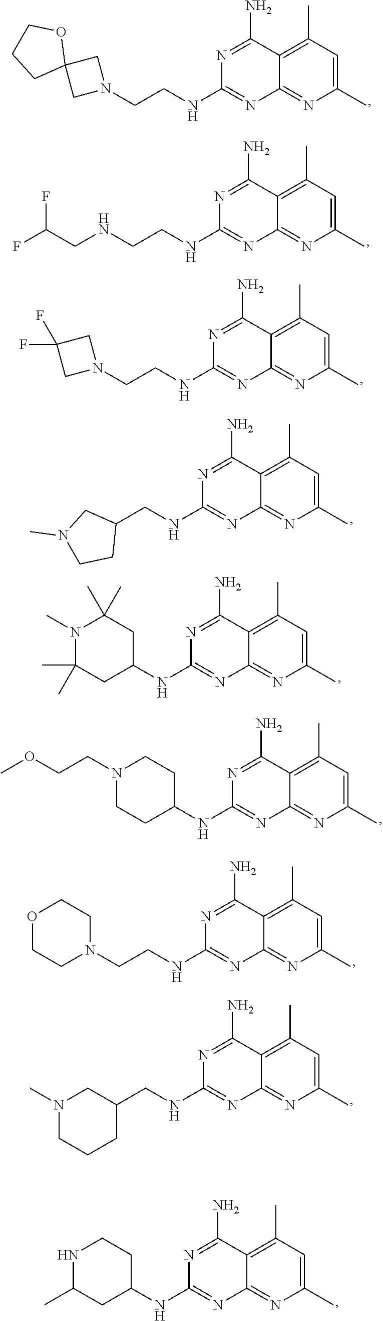

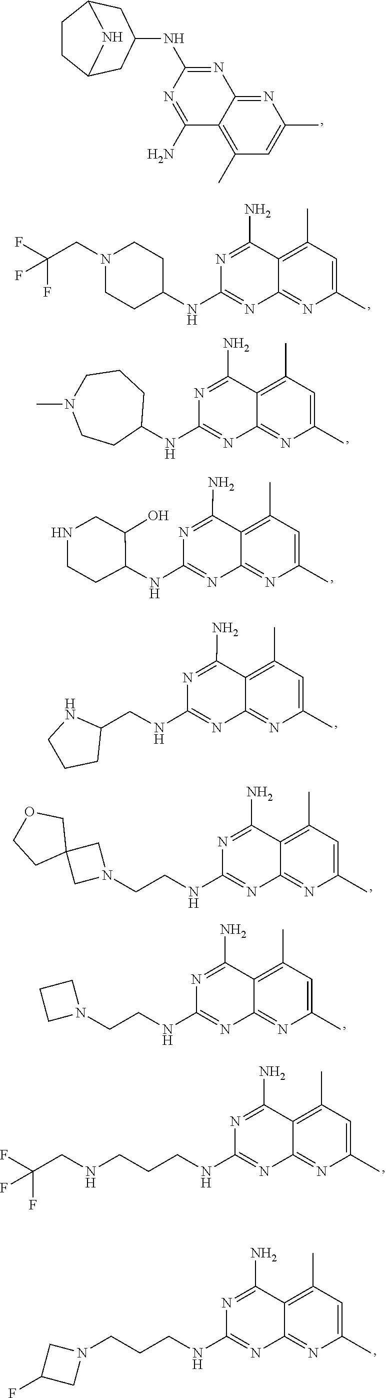

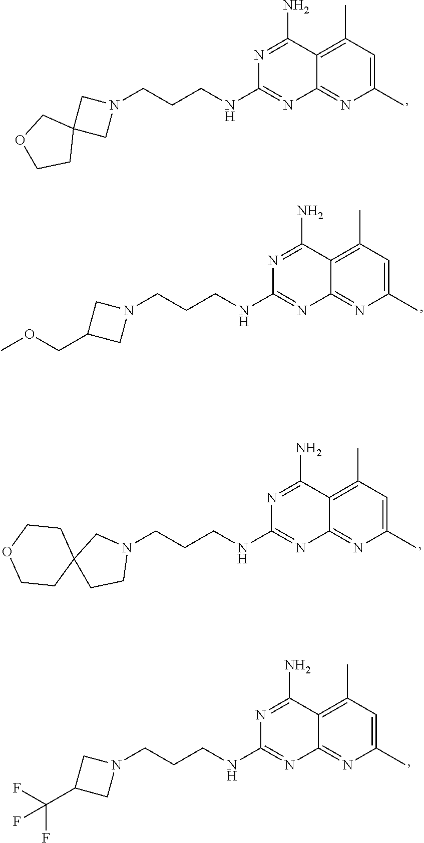

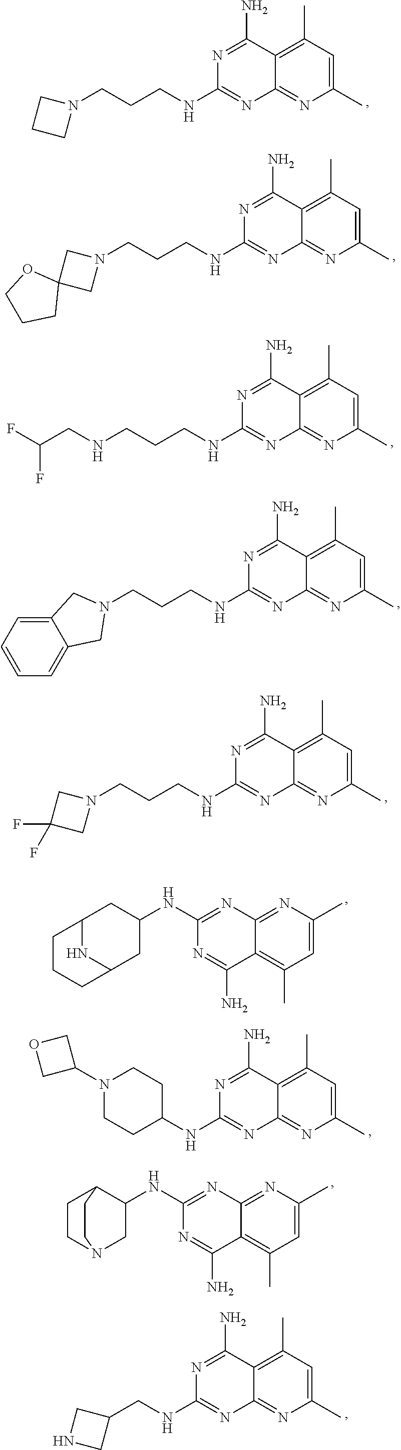

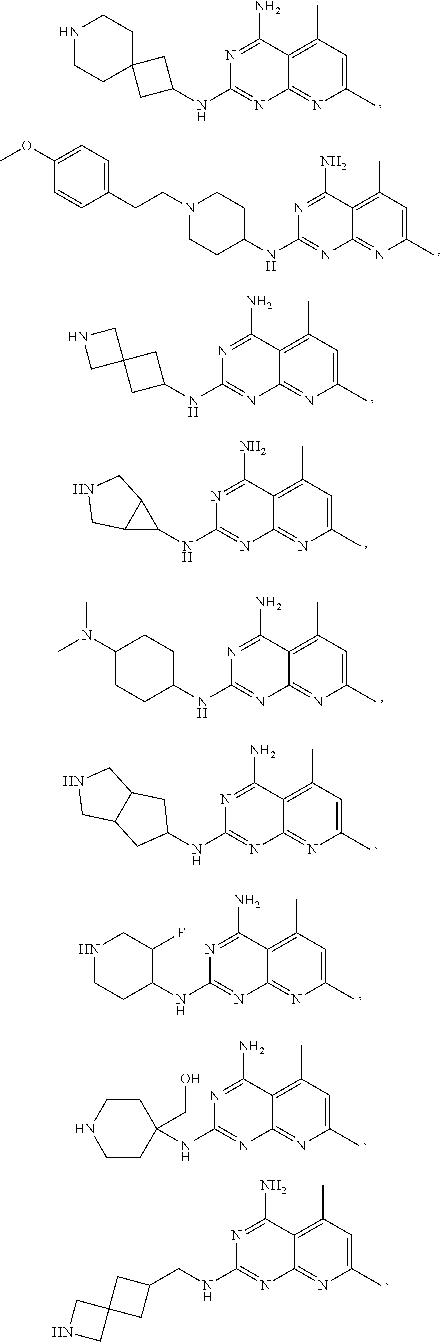

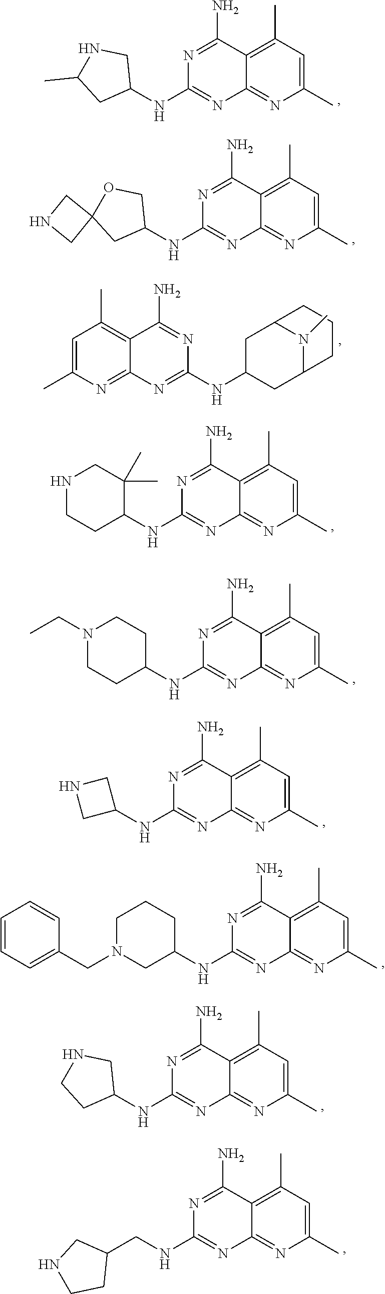

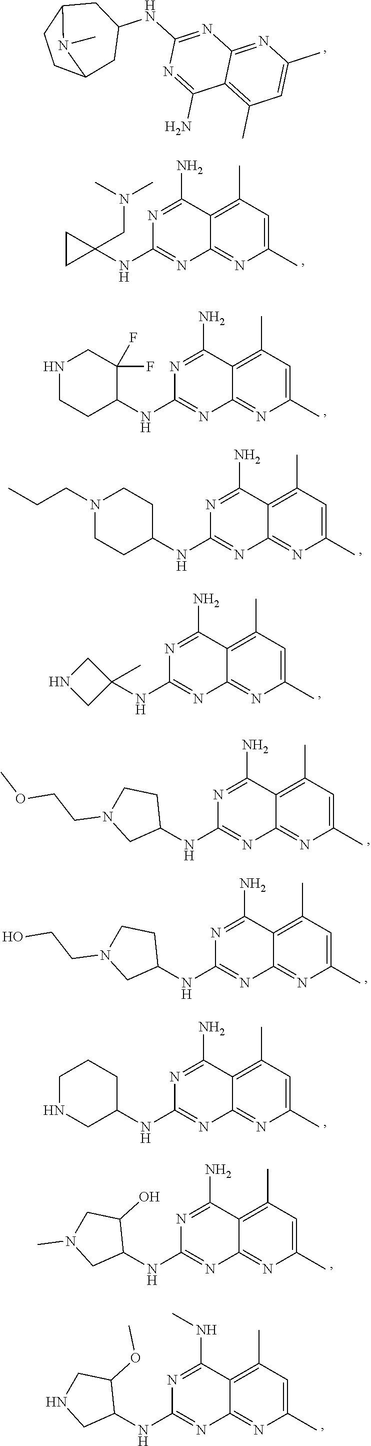

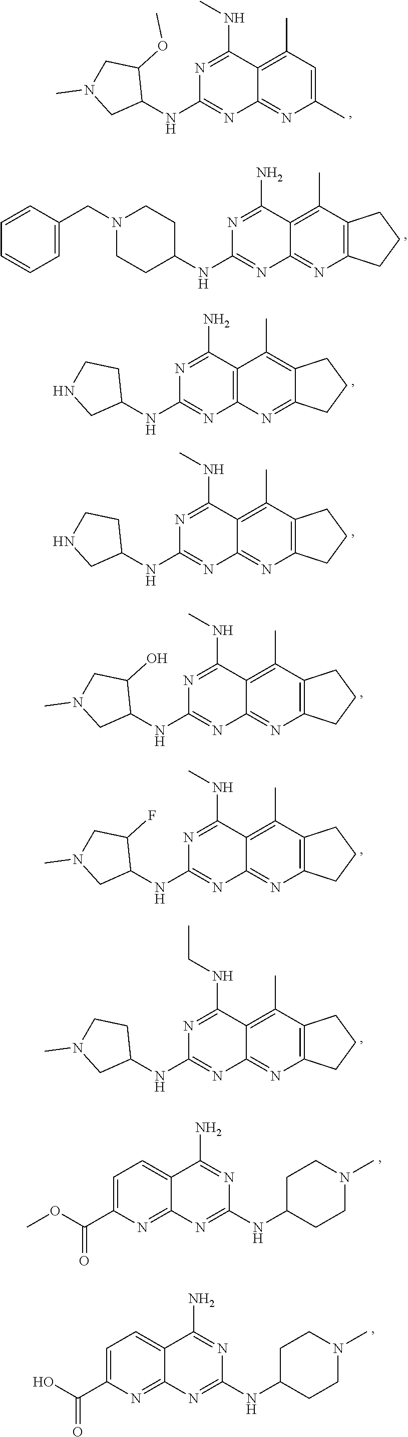

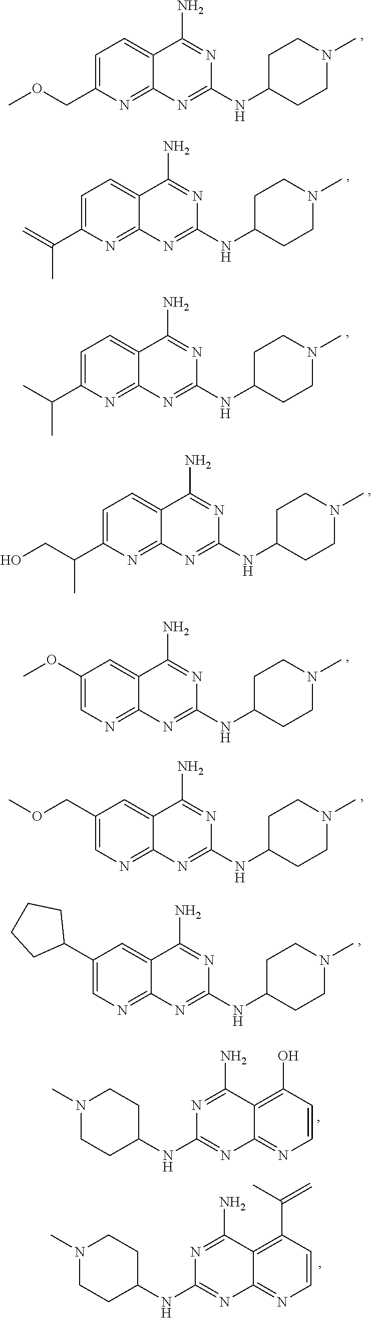

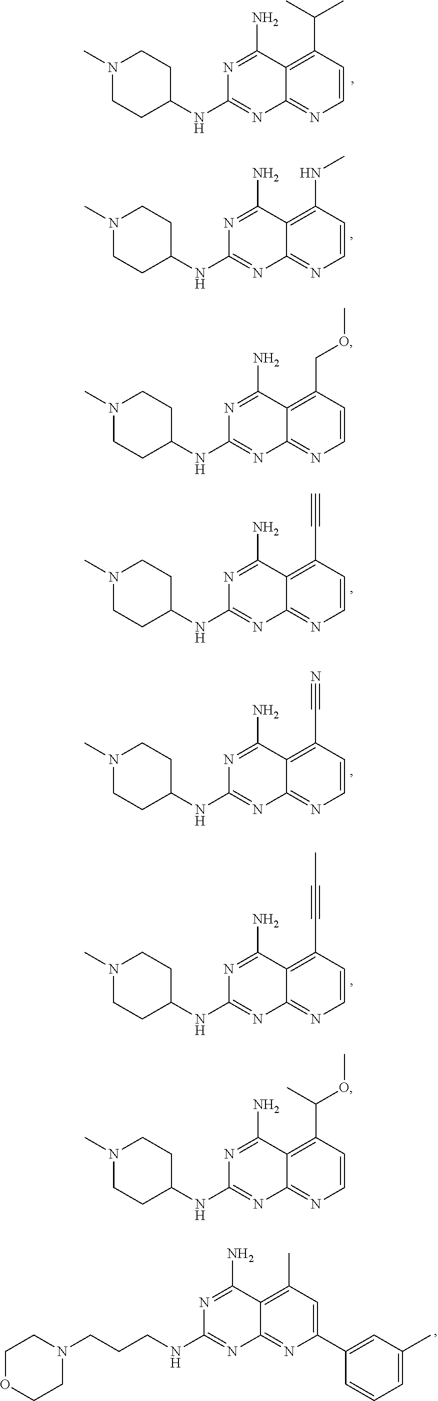

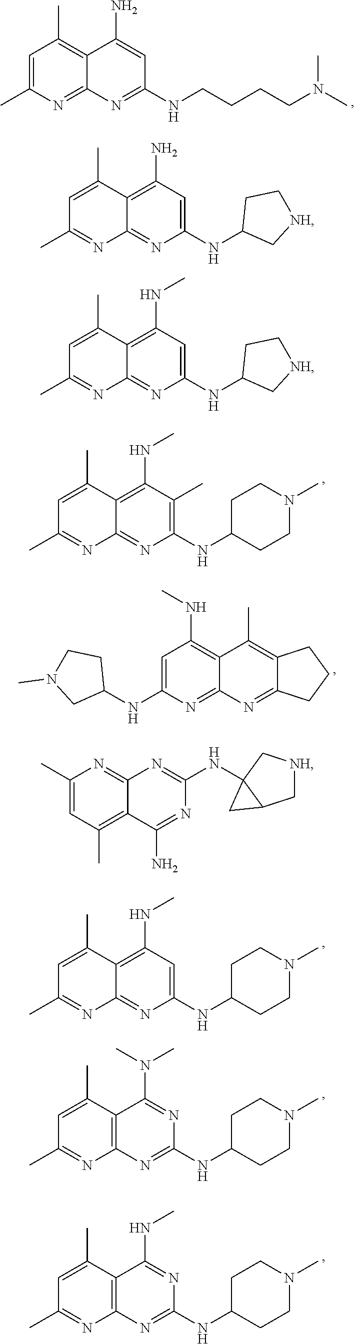

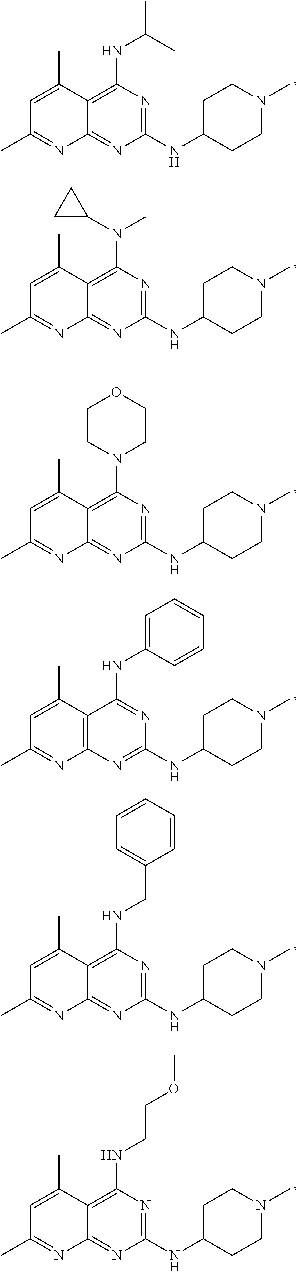

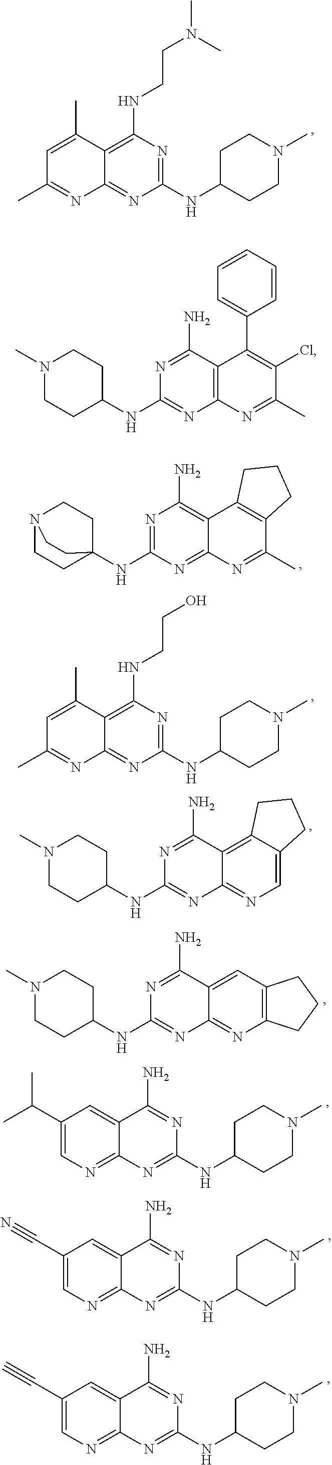

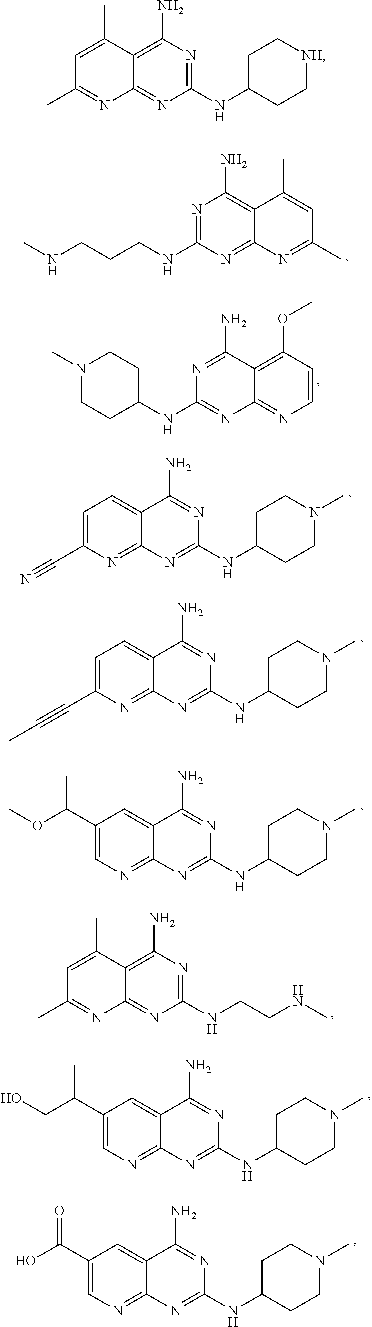

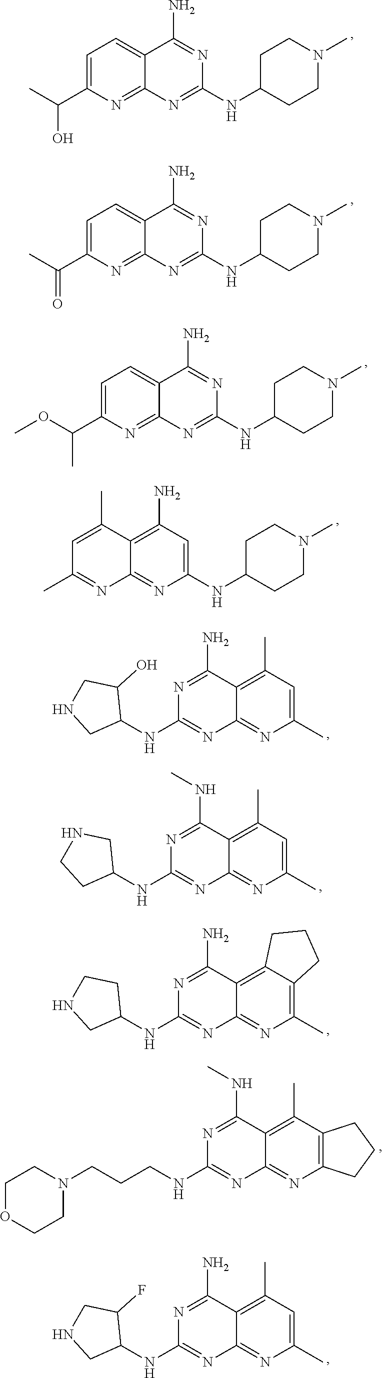

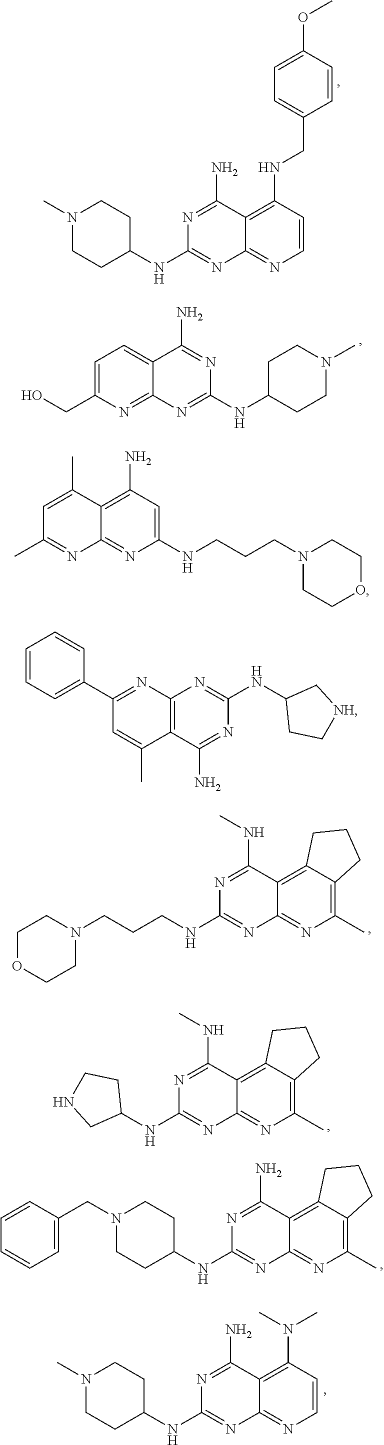

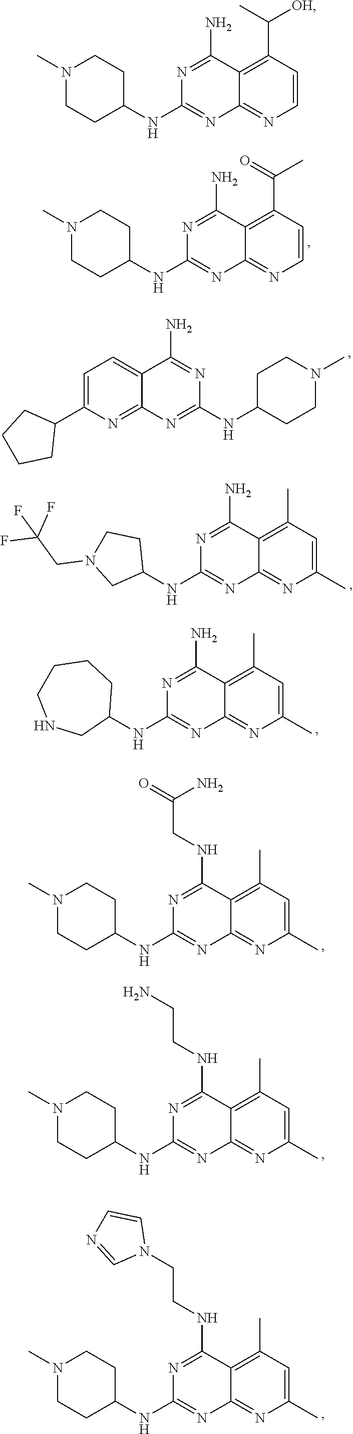

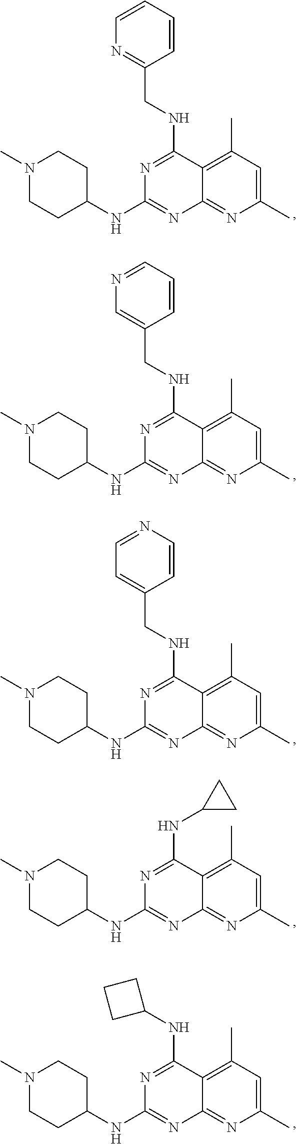

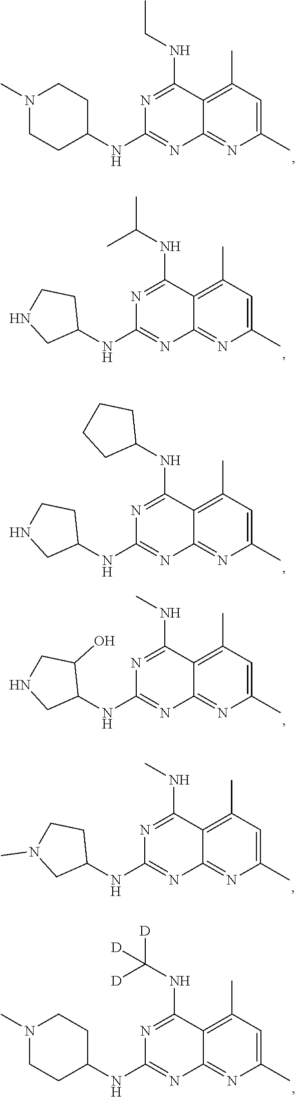

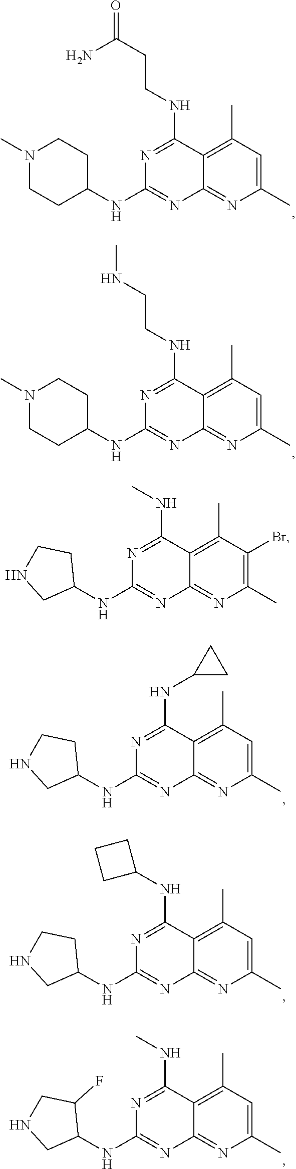

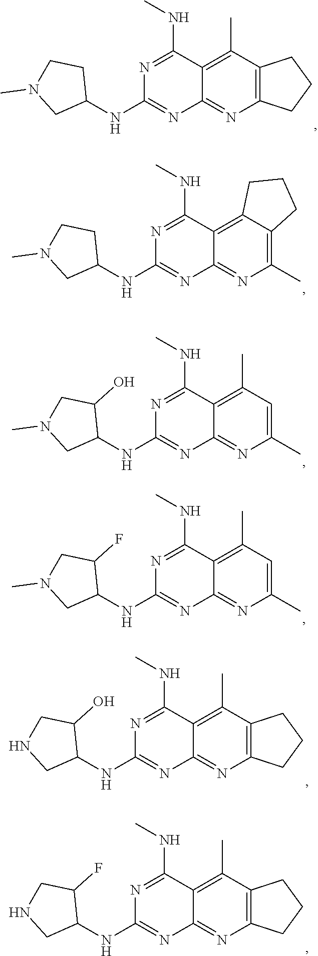

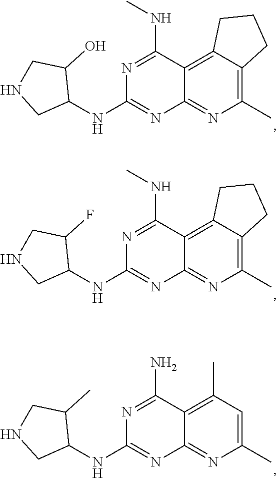 [SMILES: CNc1nc(NC2CNCC2O)nc2nc(C)c3c(c12)CCC3 ; CNc1nc(NC2CNCC2F)nc2nc(C)c3c(c12)CCC3 ; Cc1cc(C)c2c(N)nc(NC3CNCC3C)nc2n1]

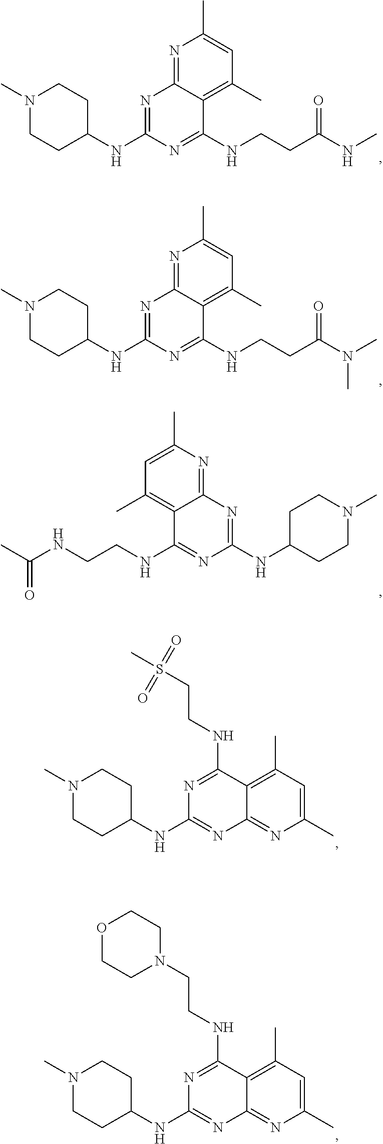

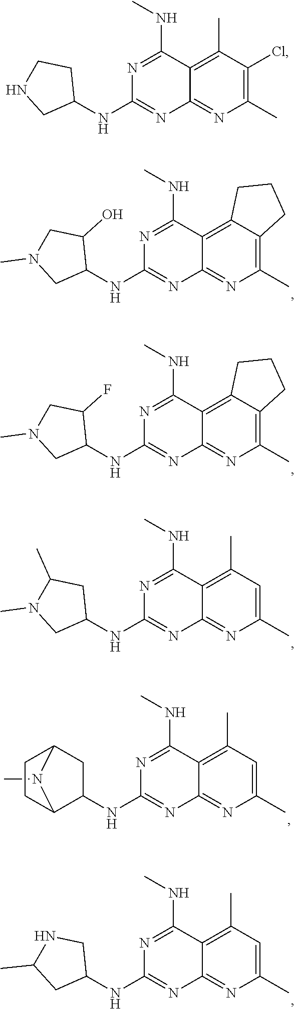

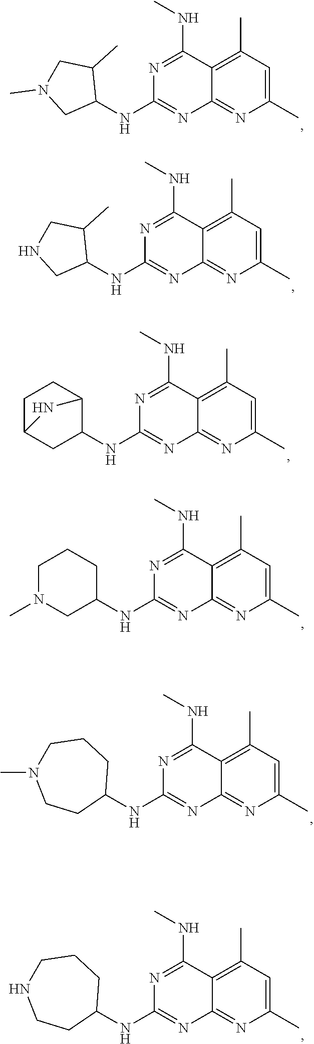

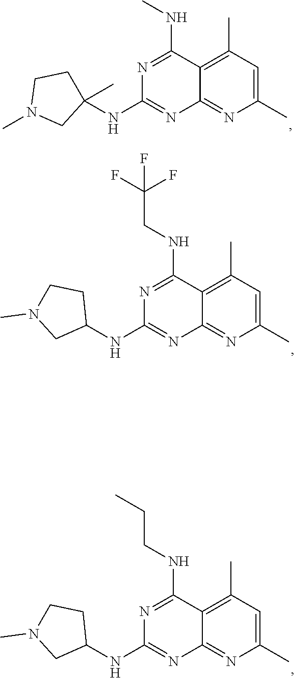

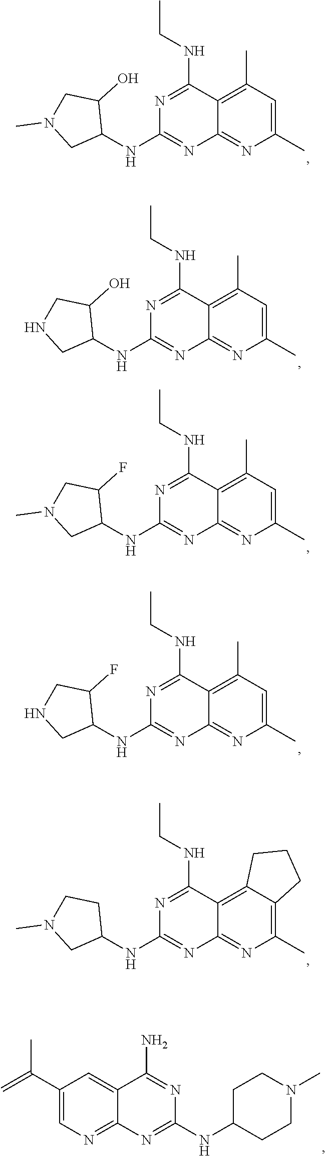

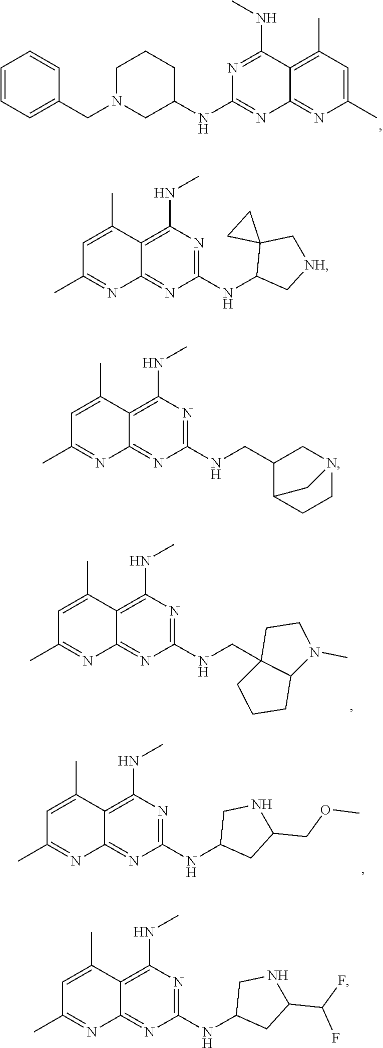

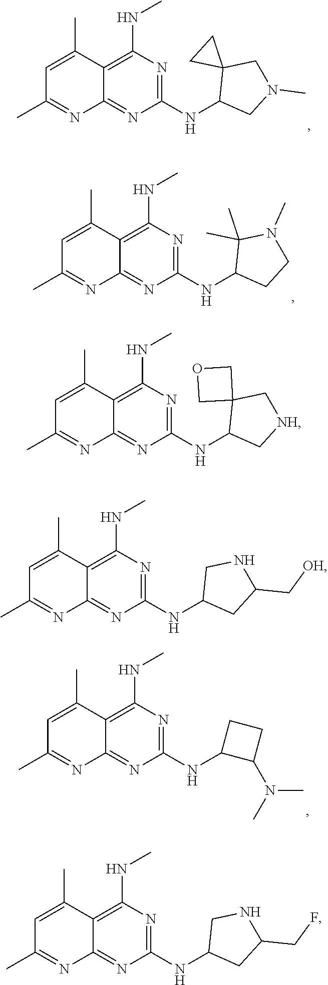

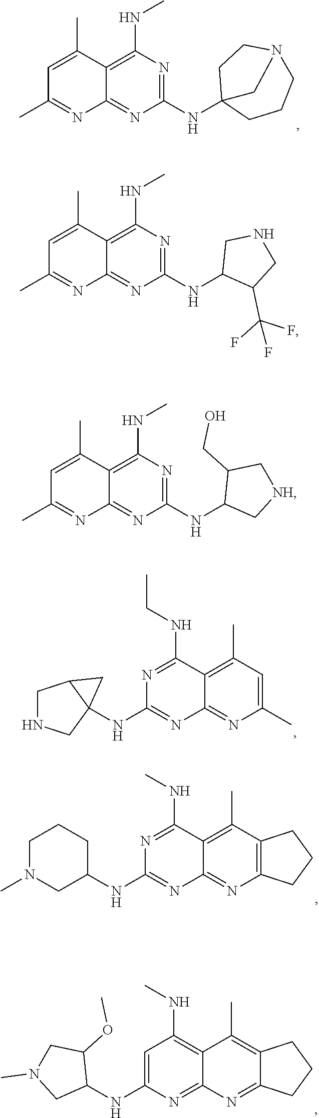

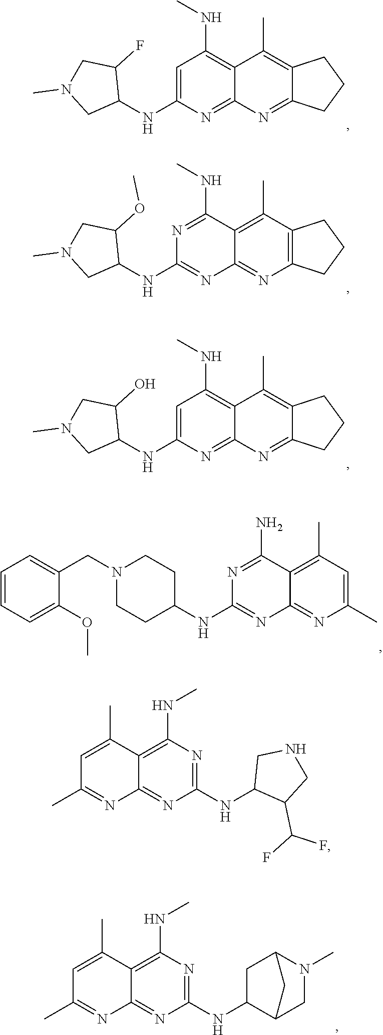

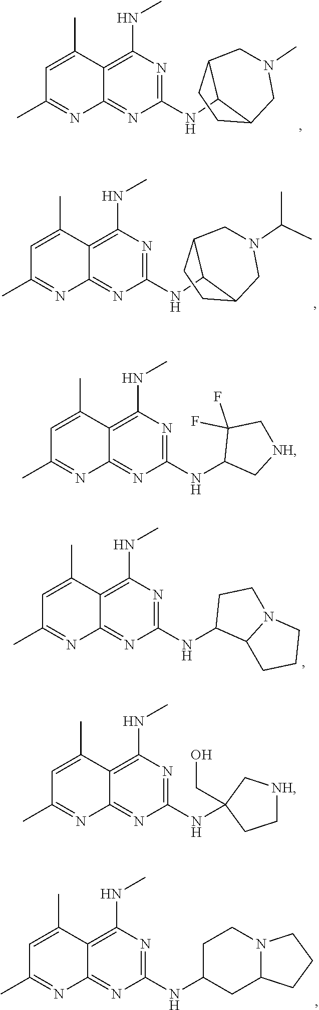

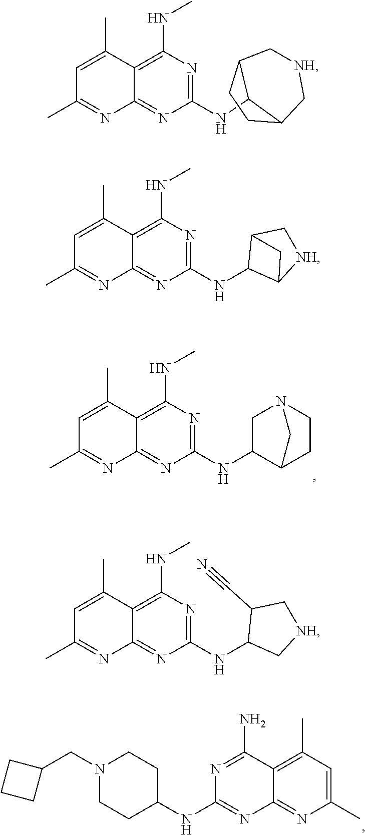 [SMILES: CNc1nc(NC2C3CCC2CNC3)nc2nc(C)cc(C)c12 ; CNc1nc(NC2C3CNC2C3)nc2nc(C)cc(C)c12 ; CNc1nc(NC2CN3CCC2C3)nc2nc(C)cc(C)c12 ; CNc1nc(NC2CNCC2C#N)nc2nc(C)cc(C)c12 ; Cc1cc(C)c2c(N)nc(NC3CCN(CC4CCC4)CC3)nc2n1]

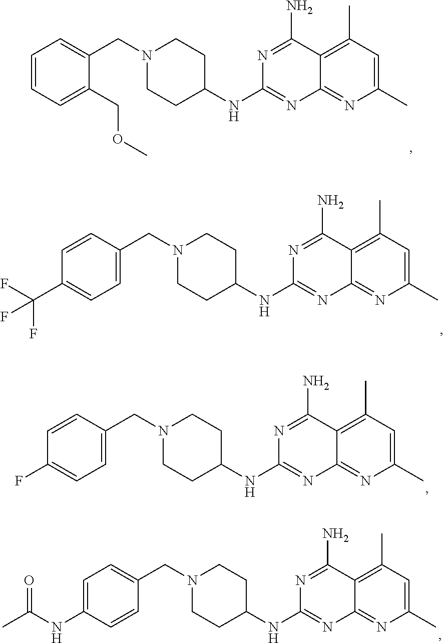

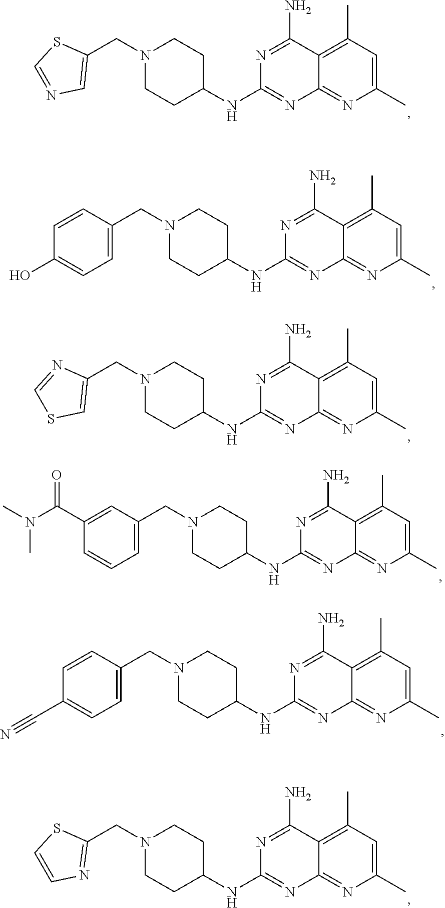

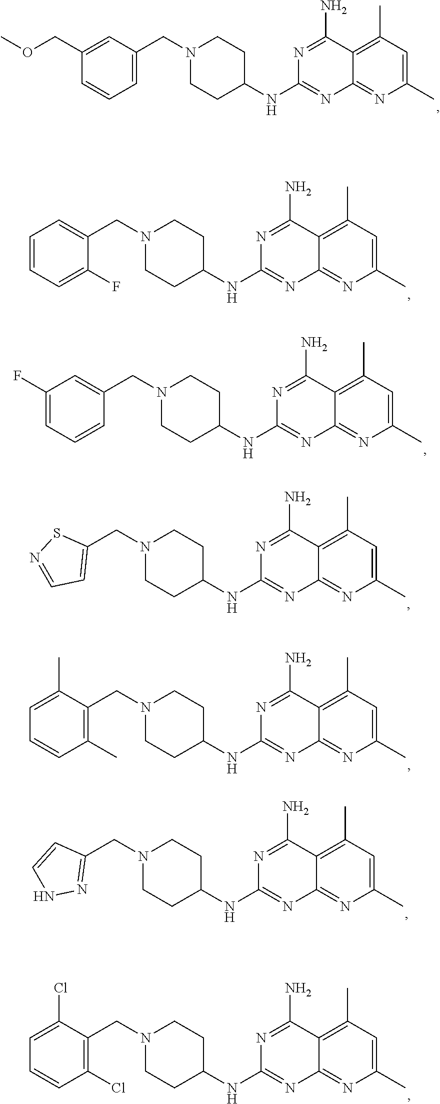

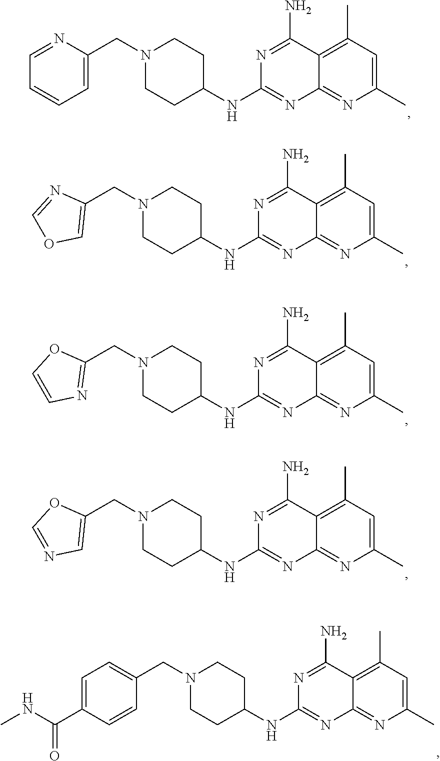

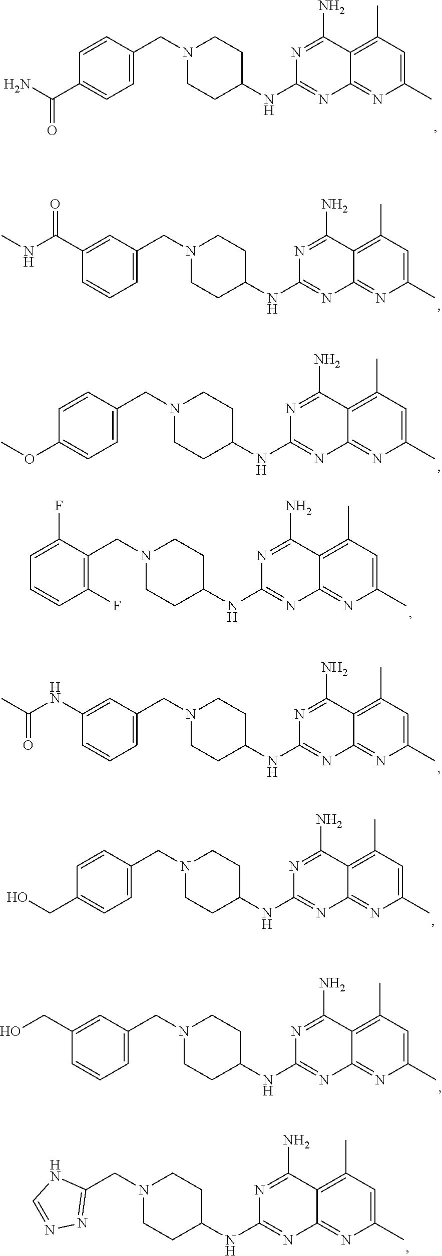

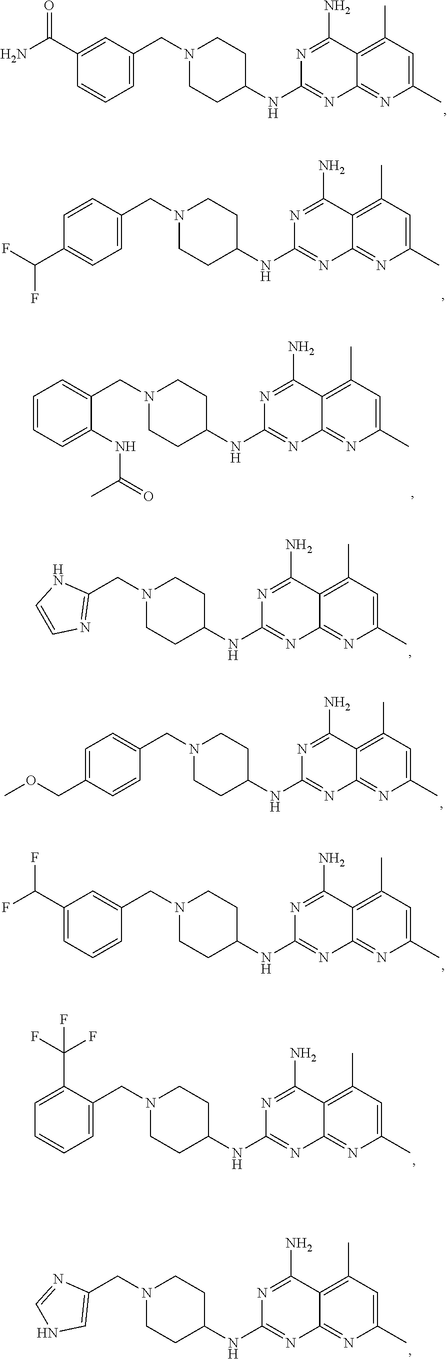

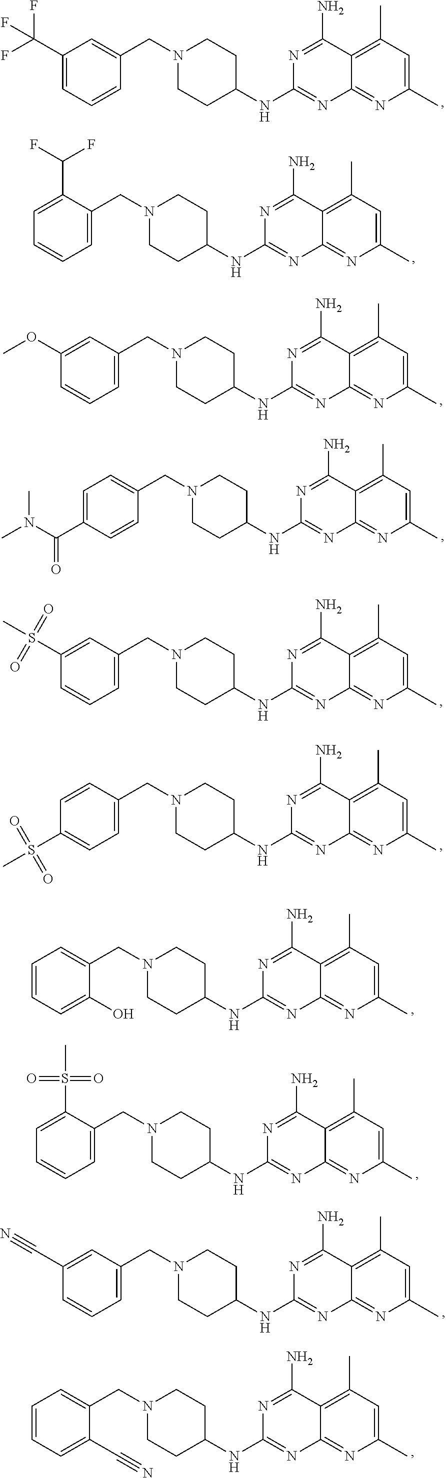

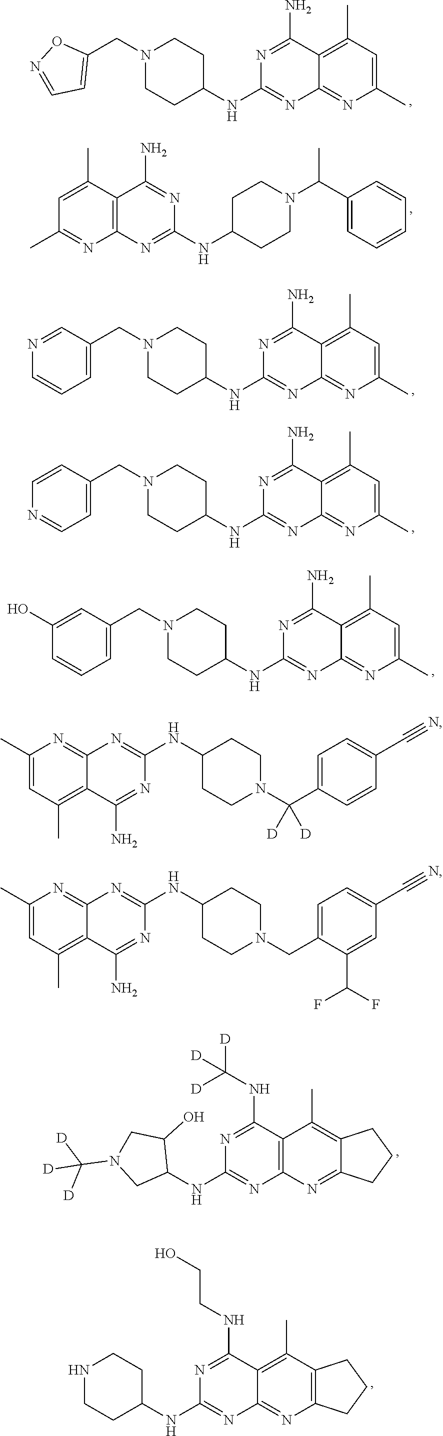

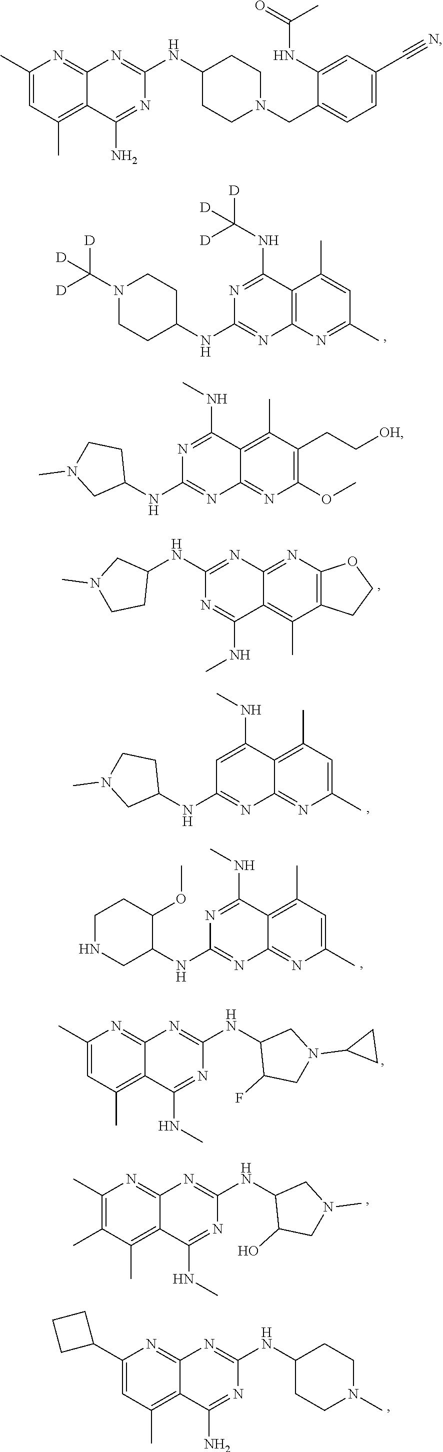

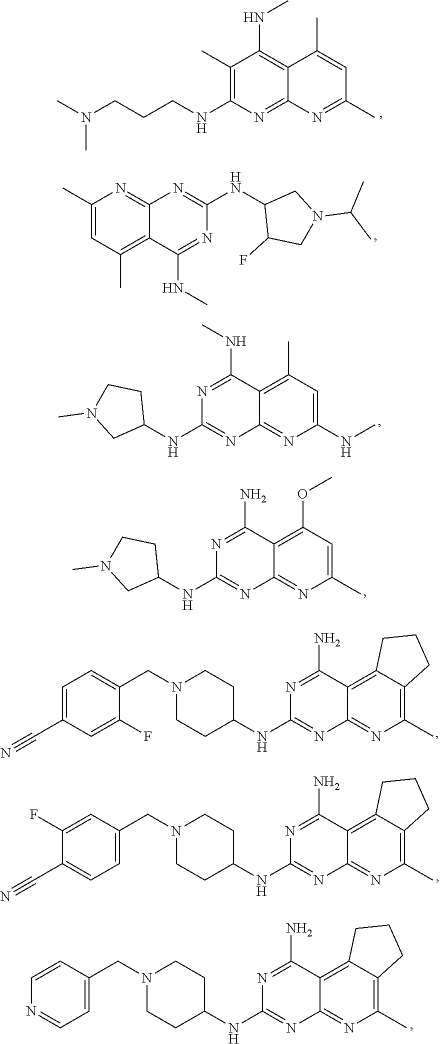

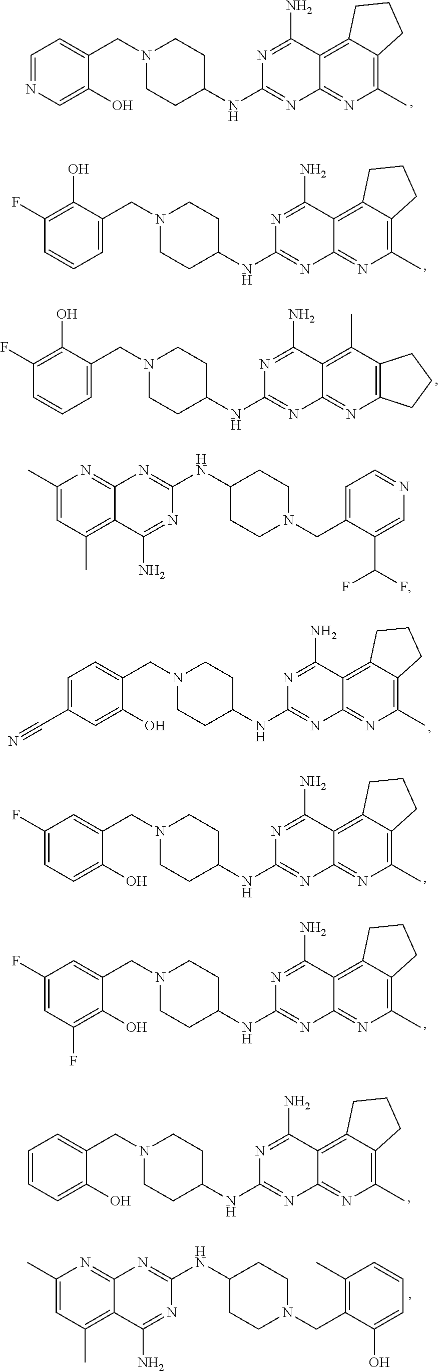

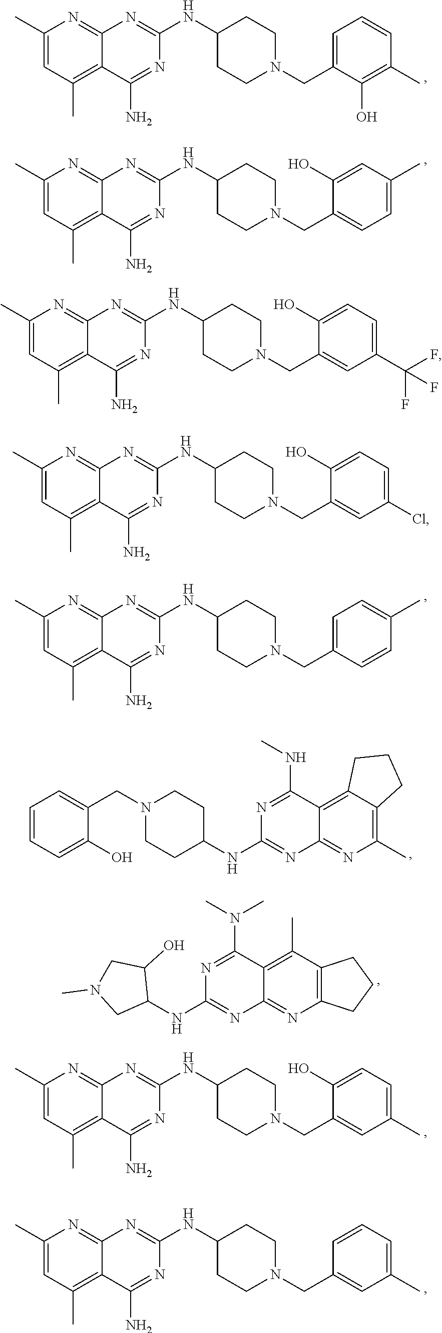

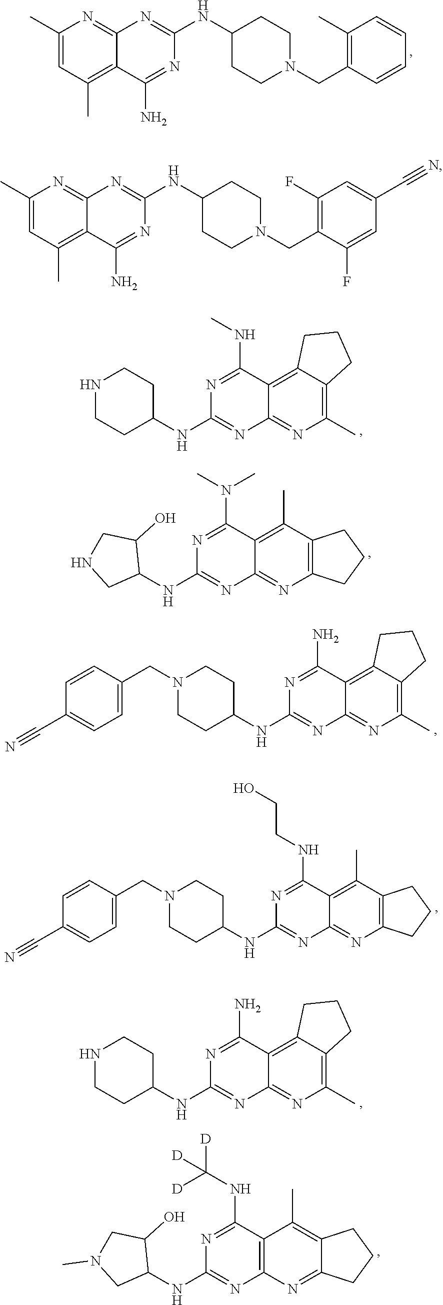

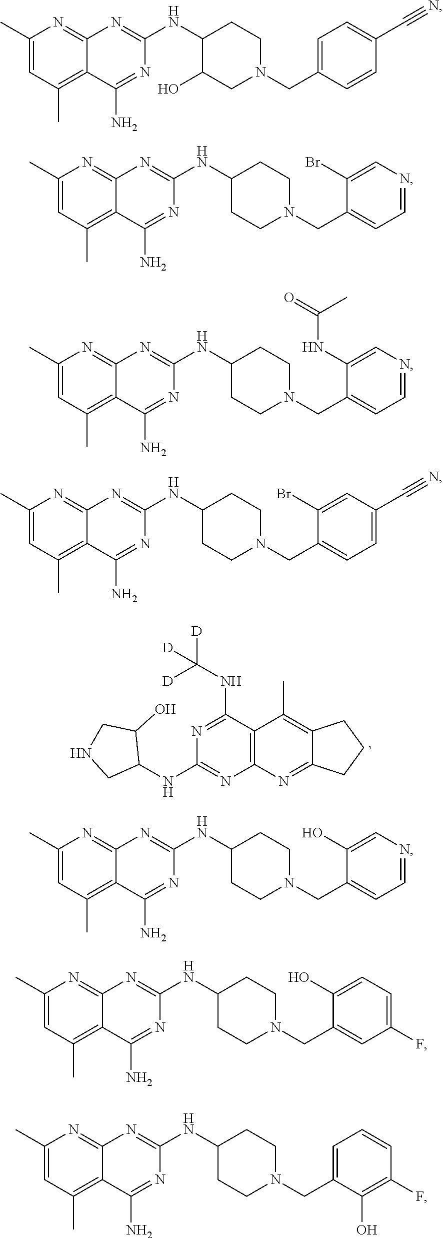

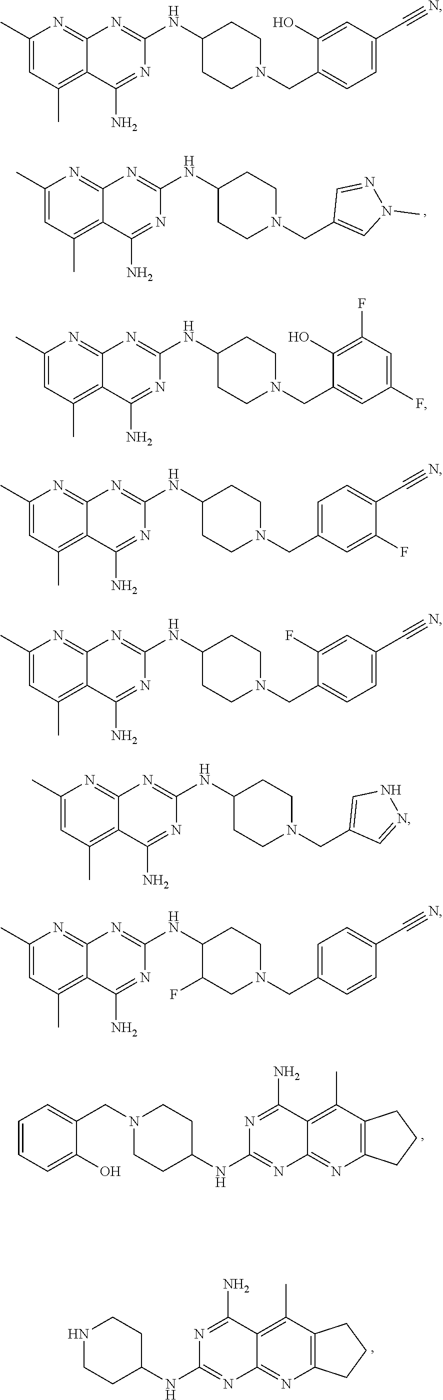

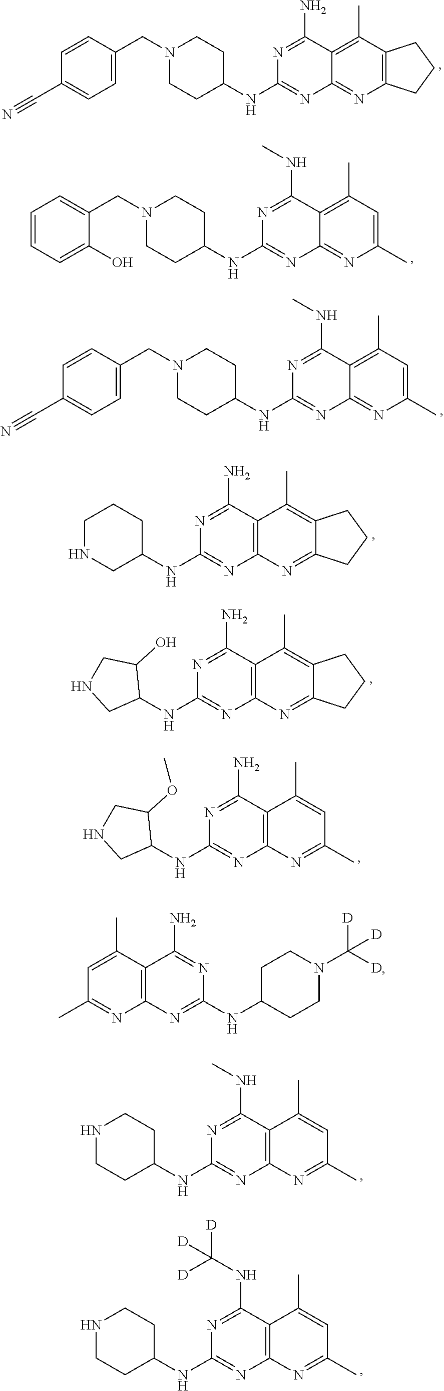

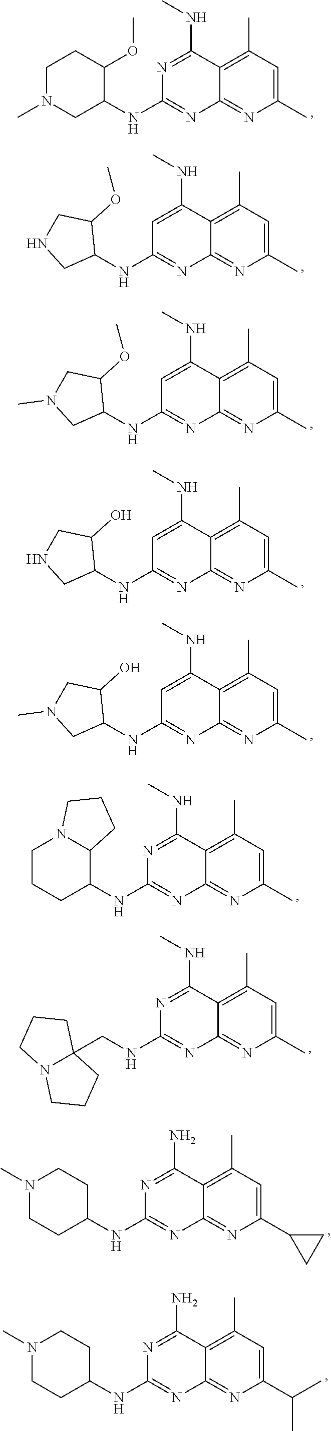

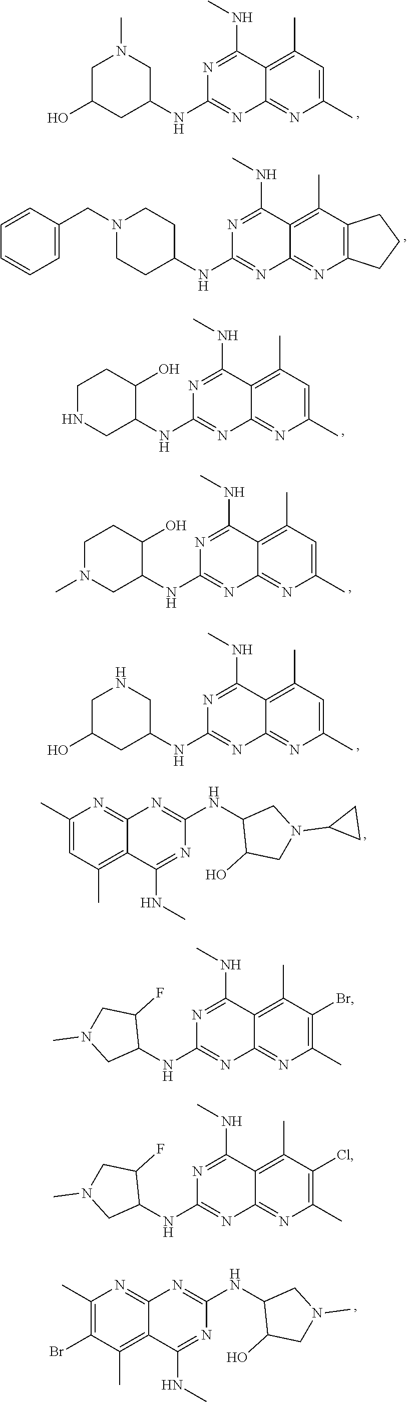

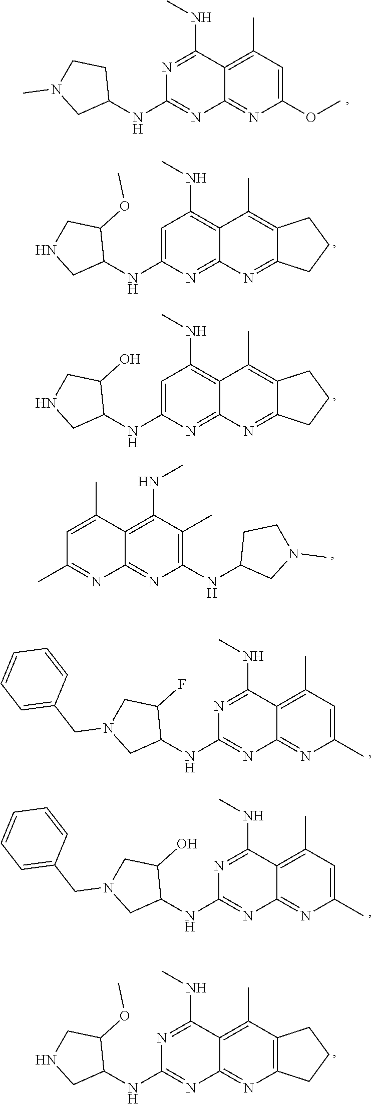

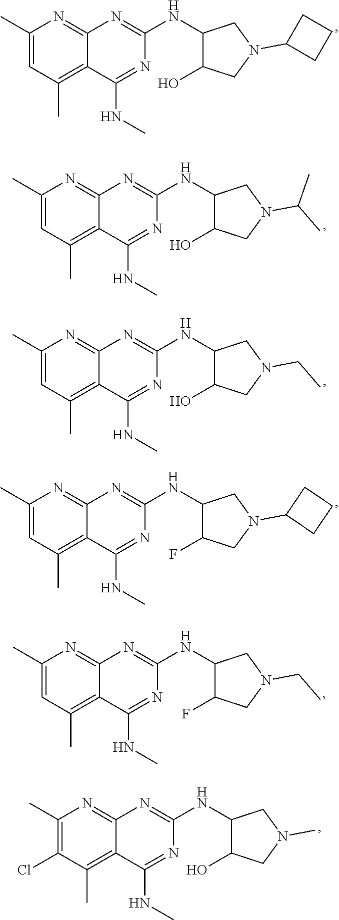

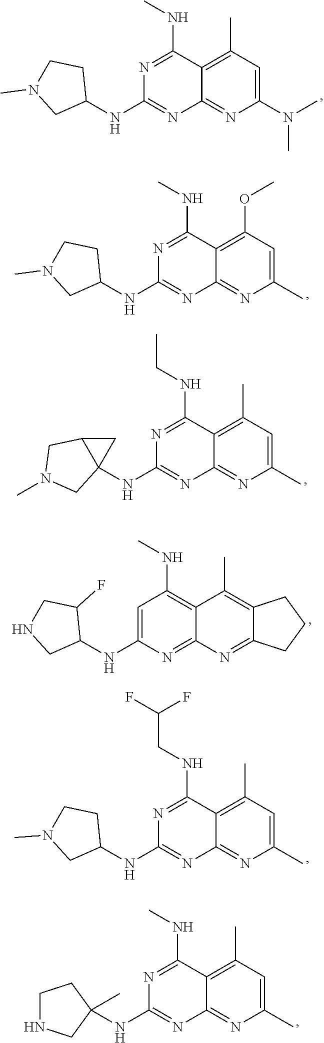

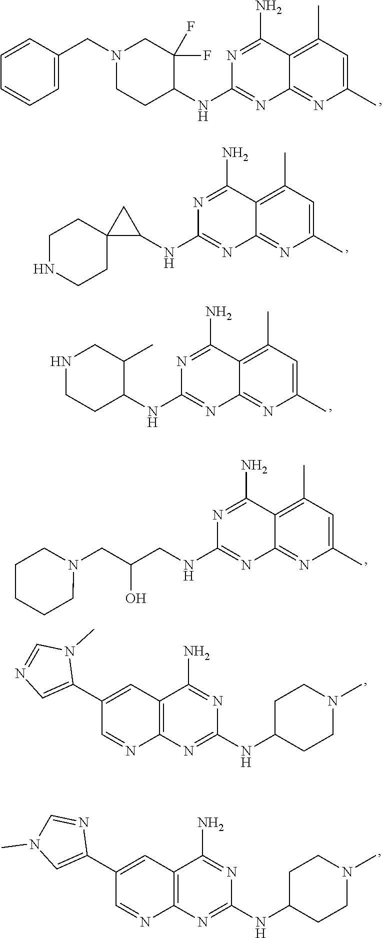

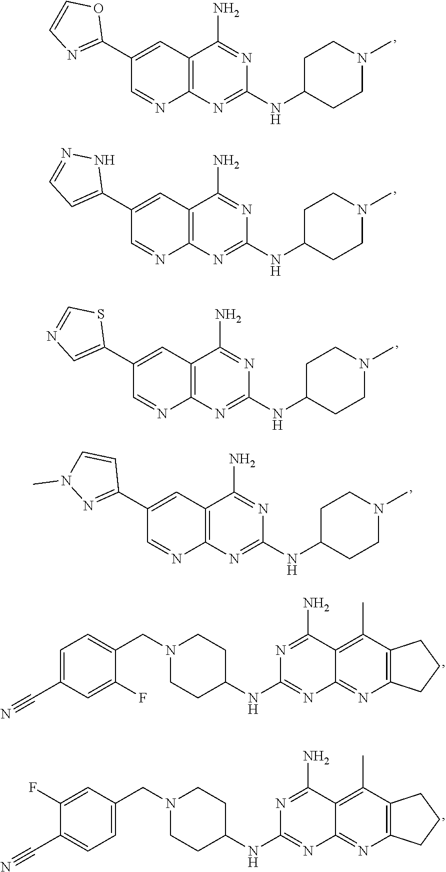

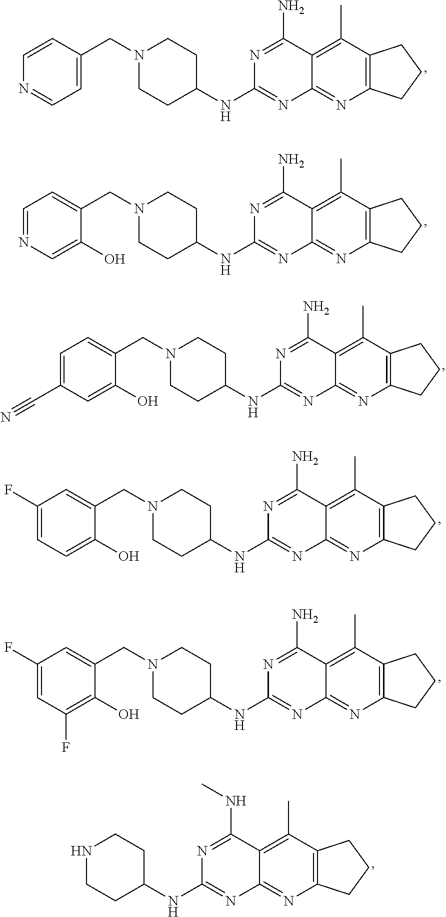 [SMILES: Cc1c2c(nc3nc(NC4CCN(Cc5ccncc5)CC4)nc(N)c13)CCC2 ; Cc1c2c(nc3nc(NC4CCN(Cc5ccncc5O)CC4)nc(N)c13)CCC2 ; Cc1c2c(nc3nc(NC4CCN(Cc5ccc(C#N)cc5O)CC4)nc(N)c13)CCC2 ; Cc1c2c(nc3nc(NC4CCN(Cc5cc(F)ccc5O)CC4)nc(N)c13)CCC2 ; Cc1c2c(nc3nc(NC4CCN(Cc5cc(F)cc(F)c5O)CC4)nc(N)c13)CCC2 ; CNc1nc(NC2CCNCC2)nc2nc3c(c(C)c12)CCC3]

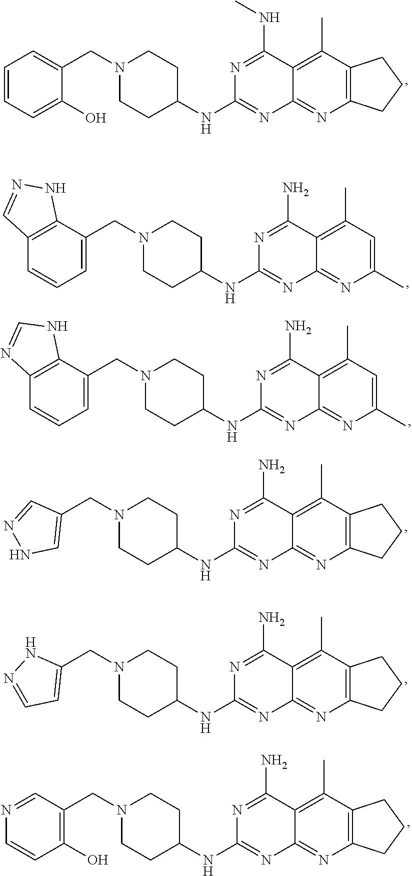

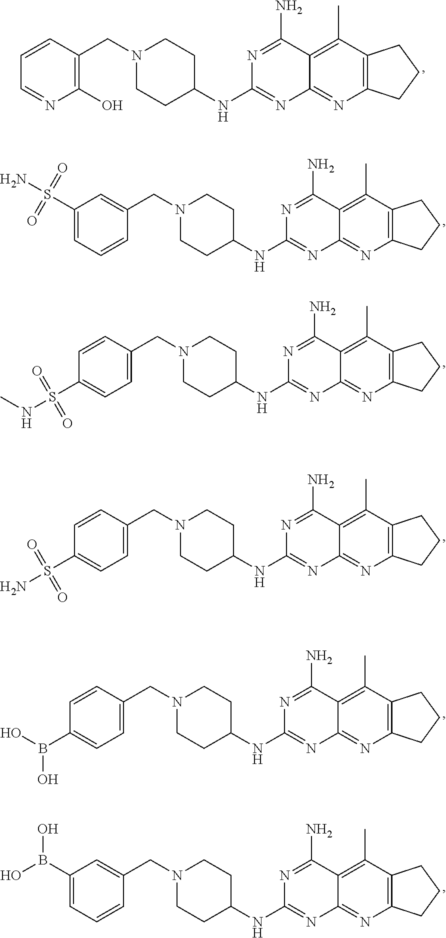 [SMILES: Cc1c2c(nc3nc(NC4CCN(Cc5cccnc5O)CC4)nc(N)c13)CCC2 ; Cc1c2c(nc3nc(NC4CCN(Cc5cccc(S(N)(=O)=O)c5)CC4)nc(N)c13)CCC2 ; CNS(=O)(=O)c1ccc(CN2CCC(Nc3nc(N)c4c(C)c5c(nc4n3)CCC5)CC2)cc1 ; Cc1c2c(nc3nc(NC4CCN(Cc5ccc(S(N)(=O)=O)cc5)CC4)nc(N)c13)CCC2 ; Cc1c2c(nc3nc(NC4CCN(Cc5ccc(B(O)O)cc5)CC4)nc(N)c13)CCC2 ; Cc1c2c(nc3nc(NC4CCN(Cc5cccc(B(O)O)c5)CC4)nc(N)c13)CCC2]

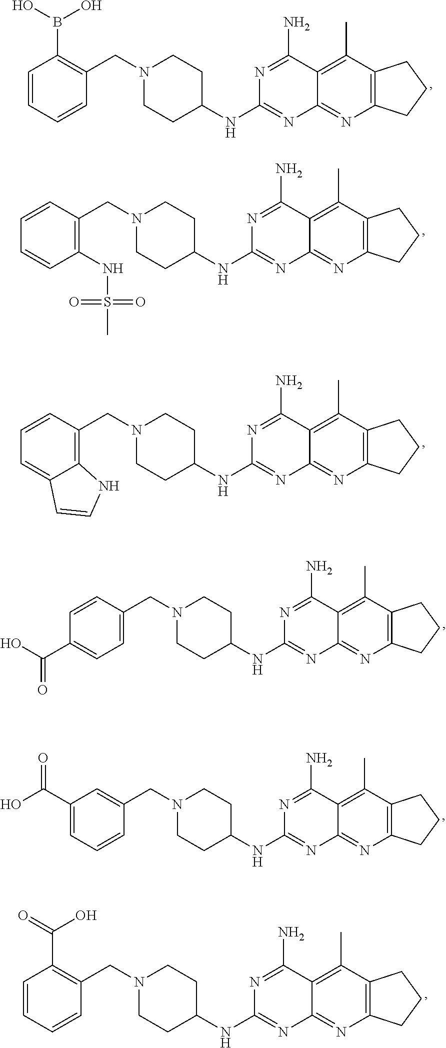 [SMILES: Cc1c2c(nc3nc(NC4CCN(Cc5ccccc5B(O)O)CC4)nc(N)c13)CCC2 ; Cc1c2c(nc3nc(NC4CCN(Cc5ccccc5NS(C)(=O)=O)CC4)nc(N)c13)CCC2 ; Cc1c2c(nc3nc(NC4CCN(Cc5cccc6cc[nH]c56)CC4)nc(N)c13)CCC2 ; Cc1c2c(nc3nc(NC4CCN(Cc5ccc(C(=O)O)cc5)CC4)nc(N)c13)CCC2 ; Cc1c2c(nc3nc(NC4CCN(Cc5cccc(C(=O)O)c5)CC4)nc(N)c13)CCC2 ; Cc1c2c(nc3nc(NC4CCN(Cc5ccccc5C(=O)O)CC4)nc(N)c13)CCC2]

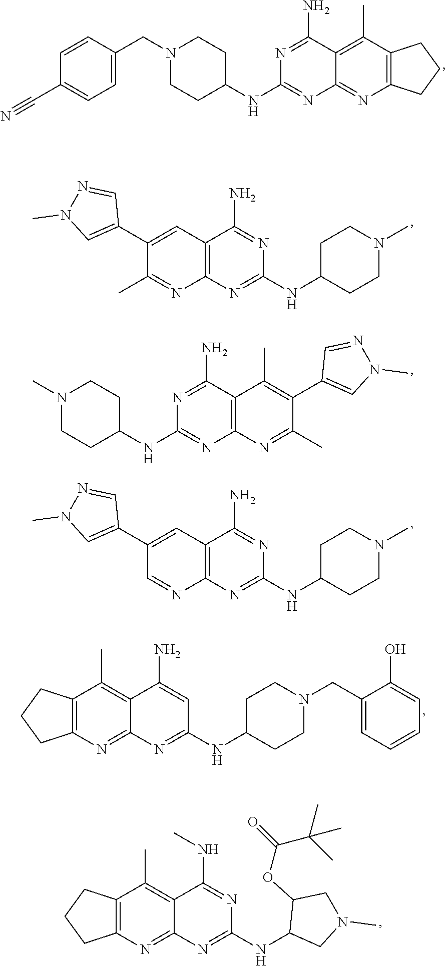

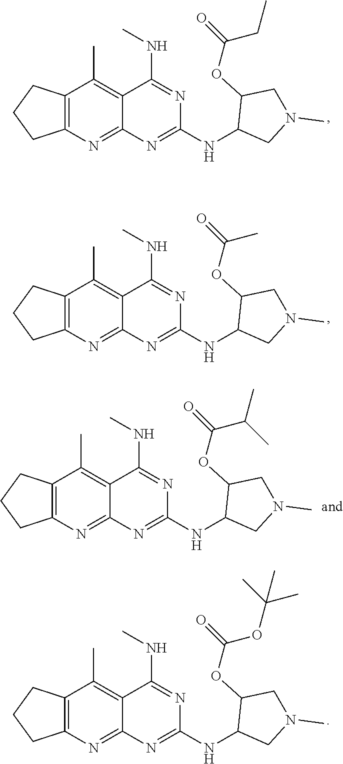

19. A pharmaceutical composition comprising a compound according to any one of claims 1 to 18, and one or more pharmaceutically acceptable excipients.

20. A compound according to any one of claims 1 to 18, or a pharmaceutical composition according to claim 19, for use as a medicament.

21. A compound according to any one of claims 1 to 18, or a pharmaceutical composition according to claim 19, for use in the treatment of: a disease of the musculoskeletal system, a disease of the skin, a metabolic disease, a disease of the Nervous System, a Cardiovascular disease, an Endocrine disorder, a disease of the eye, a disease affecting the urogenital system, a haemic or lymphatic condition, a respiratory disease, an inflammatory or autoimmune condition, a disease of the Gastrointestinal system, a Neoplasm, cancer, or a disease or disorder selected from Amelogenesis Imperfecta, Anodontia, Odontodysplasia, Branchio-Oto-Renal Syndrome, Sotos Syndrome, and Waardenburg's Syndrome.

22. A compound according to any one of claims 1 to 18, or a pharmaceutical composition according to claim 19, for use in the treatment of a disease selected from: Recessive dystrophic epidermolysis bullosa, Junctional epidermolysis bullosa, Xeroderma pigmentosum, Netherton syndrome, Duchenne Muscular Dystrophy, Becker Muscular Dystrophy, Cystic Fibrosis, Dravet Syndrome, Aniridia, Methylmalonic Acidemia, Colorectal Cancer, Endometrium Cancer, Breast Cancer, Ovarian Cancer, Lung Squamous Cell Carcinoma, Head and Neck Squamous Cell Carcinoma, Familial adenomatous polyposis, Hemophilia A, Hemophilia B, Choroideremia, Pulmonary Artery Hypertension, Ataxia telangiectasia, Shwachman-Diamond syndrome, Mucopolysaccharidosis Type I, Mucopolysaccharidosis Type VI, Mucopolysaccharidosis type III, Niemann-Pick Disease, Primary Ciliary Dyskinesia, Usher syndrome and Retinitis Pigmentosa.

23. A compound according to any one of claims 1 to 18, or a pharmaceutical composition according to claim 19, for use in a method of treating conditions or disorders which are associated with a PTC mutation in a subject.
